(19) United States
(12) Patent Application Publication
(10) Pub. No.: US 2026/0216070 A1
(43) Pub. Date: Jul. 30, 2026

(54) COMPOUNDS AND COMPOSITIONS FOR INTRACELLULAR DELIVERY OF NUCLEIC ACID-BASED THERAPEUTICS AND METHODS THEREOF

(71) Applicant: NanoTech Pharma Inc., Hillsborough, NJ (US)

(72) Inventors: Xian Xu, Monmouth Junction, NJ (US); Robert Crovak, Langhorne, PA (US); Huabin Wu, Millington, NJ (US); Zheng Yue, Bridgewater, NJ (US); Fang Liu, Flemington, NJ (US)

(73) Assignee: NanoTech Pharma Inc., Hillsborough, NJ (US)

(21) Appl. No.: 19/109,937

(22) PCT Filed: Sep. 8, 2023
(86) PCT No.: PCT/US2023/032359

§ 371 Date: Mar. 7, 2025

Related U.S. Application Data
(60) Provisional application No. 63/375,007, filed on Sep. 8, 2022.
(60) Provisional application No. 63/503,714, filed on May 22, 2023.

(51) Int. Cl. A61K 9/1271; A61K 9/51; A61K 31/7105; C07C 211/09; C07C 211/25; C07C 229/16; C07C 229/18; C07C 229/24; C07C 323/12; C07C 323/25; C07C 323/26; C07D 207/06; C07D 207/14; C07D 211/26; C07D 233/61; C07D 239/10; C07D 263/32; C07D 277/28; C07D 295/13; C07D 295/24; C07D 309/04; C07D 401/12; C07D 413/06; C07D 487/04; C07D 487/14; C12N 15/88

(52) U.S. Cl. CPC A61K 9/1271; A61K 9/5123; A61K 31/7105; C07C 211/09; C07C 211/25; C07C 229/16; C07C 229/18; C07C 229/24; C07C 323/12; C07C 323/25; C07C 323/26; C07D 207/06; C07D 207/14; C07D 211/26; C07D 233/61; C07D 239/10; C07D 263/32; C07D 277/28; C07D 295/13; C07D 295/24; C07D 309/04; C07D 401/12; C07D 413/06; C07D 487/04; C07D 487/14; C12N 15/88; C07C 2601/08; C07C 2601/14

(57) ABSTRACT

The present disclosure provides compounds according to Formula I, II, III, IV, V, VI, VII, VIII, IX, X, XI, XII, XIII, and XIV used as a component of lipid nanoparticle compositions for the delivery of a biological and/or therapeutic agent. The present disclosure also provides novel, stable lipid nanoparticle compositions comprising one or more biological and/or therapeutic agents, methods of making the lipid nanoparticle compositions, and methods of delivering the lipid nanoparticle composition.

### CROSS-REFERENCE TO RELATED APPLICATIONS

This application claims priority under 35 U.S.C. § 119(e) to U.S. Provisional Patent Application No. 63/375,007, filed on Sep. 8, 2022, and Application No. 63/503,714, filed on May 22, 2023, the disclosures of which are incorporated herein by reference in their entireties.

### FIELD OF THE DISCLOSURE

This disclosure relates to compounds and lipid nanoparticle (LNP) compositions thereof, as well as methods of using these compounds and LNP compositions for transfection and delivery of biological and therapeutic agents, such as nucleic acid molecules, to cells.

### BACKGROUND OF THE DISCLOSURE

Novel therapies are needed for the treatment of protein deficiency or misfolding related diseases, for example, Alzheimer's disease, Parkinson's disease, cystic fibrosis, Fabry disease, etc. For certain protein related diseases, there is a need for replacement of a protein or enzyme that is normally secreted by cells. Gene therapies could provide a treatment or even cure of such disorders; however, there have been several limitations to using conventional gene therapies for this purpose.

One major obstacle in prior approaches at delivery of nucleic acids encoding proteins is that it is often difficult to achieve significant levels of the desired protein and the amounts are not sustained over time, resulting in the requirement of multiple booster dosages. Another major obstacle in conventional gene therapies is that once the DNA is introduced into the cell, only a small portion is integrated into the genome. This integration may provide long lasting benefits, but it may also cause delirious effects, such as inducing a mutation that impedes or eliminates the function of the endogenous gene.

In contrast, mRNA-based therapies eliminate the risk of inducing genome altering mutations and any delirious effects would be of a limited duration due to the relatively short half-life of RNA. Additionally, mRNA does not need to enter the cell nucleus to perform its intended function, significantly easing the challenges of conventional gene therapies.

Delivery of mRNA to cells poses a significant challenge due to its inherent instability. mRNA lacks the more stable double helix structure of DNA due to steric hindrance caused by the presence of 2-hydroxyl groups on the ribose sugars. This makes it more prone to hydrolytic degradation. Additionally, once the mRNA reaches the cytoplasm of the cell, it is exposed to degrading enzymes (RNases). Presently mRNA-based solutions and products need to be stored at ultra-low temperatures (e.g., −80° C.) and in the absence of ubiquitous RNases.

It has been shown that encapsulation of mRNA inside of a particle comprised of lipids (lipid nanoparticles, LNPs) significantly increases the stability of mRNA as well as protecting it from RNases and facilitating transfection (see e.g., Nat. Rev. Mater. 2021, 6, 1078-1094). Early versions of LNPs, comprised of a cationic lipid (DOTAP, DOTMA, DDAB, etc.), helper lipid (HSPC, DSPC, etc.), cholesterol, and a PEG modified lipid, proved to be toxic because of the permanent cation in the cationic lipid. Additionally, these early formulations did not degrade in the body fast enough, leading to accumulation after repeated dosage.

The replacement of cationic lipids with amino ionizable lipids in the lipid nanoparticle composition have shown a marked decrease in toxicity, leading to the development of novel RNA based therapies (e.g., Alnylam's Onpattro, Pfizer/BioNTech's Comirnaty, and Moderna's Spikevax). Amino ionizable lipids include, for example, amine containing lipids that can be readily protonated under physiological conditions.

However, there remains a significant need for more effective compositions for the introduction of mRNA into cells to improve their biological performance. In addition, there is a need for new formulations to deliver nucleic acid-based therapies. The present disclosure addresses these and other needs.

### SUMMARY OF THE DISCLOSURE

The present disclosure provides novel compounds and compositions to facilitate intracellular delivery of biologically active and/or therapeutic molecules. The present disclosure also provides methods of making the particles and methods for delivery and/or administering the particles (for treatment of a disease or disorder).

In one aspect of the disclosure, the compounds described herein are of Formula I, or a salt or isomer thereof:

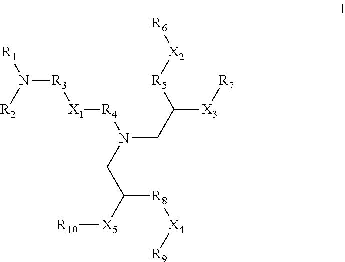

wherein:
R₁ and R₂ are the same or different, each independently alkyl, alkenyl, or alkynyl; or alternatively R₁ and R₂ together with the nitrogen atom to which they are attached form a heterocyclyl;
R₃ is a C₁₋₁₈ alkylene, C₂₋₁₈ alkenylene, or C₂₋₁₈ alkynylene, or together with the adjacent nitrogen atom forms a ring structure comprising 3-18 carbon atoms;
R₄ is a C₁₋₁₈ alkylene, C₂₋₁₈ alkenylene, or C₂₋₁₈ alkynylene;
R₅ and R₈ are the same or different, each independently a bond, or a C₁₋₂₈ alkylene, C₂₋₂₈ alkenylene, or C₂₋₂₈ alkynylene;
R₆ and R₉ are the same or different, each independently hydrogen or a linear C₁₋₂₈ alkyl or C₂₋₂₈ alkenyl;
R₇ and R₁₀ are the same or different, each independently hydrogen or a linear C₁₋₂₈ alkyl or C₂₋₂₈ alkenyl;
X₁ is a bond, —O—, —CO—, —OC—O—, or —O—CO—;
X₂ and X₄ are the same or different, each independently methylene (—CH₂—), —S—, —S—S—, —O—, —O—CO—, —CO—O—, or —NR—, wherein R is a lower alkyl; and
X₃ and X₅ are the same or different, each independently a methylene (—CH₂—), —S—, —S—S—, —O—, —O—CO—, —CO—O—, or —NR—, wherein R is a lower alkyl,
wherein the longest chain of atoms in the compound is between 18 to 70 atoms.

In another aspect of the disclosure, the compounds described herein are of Formula II, or a salt or isomer thereof

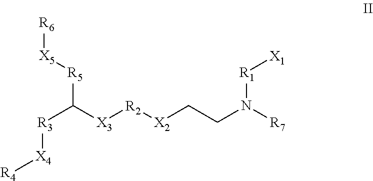

wherein:
R₁ is a C₁₋₁₈ alkylene, C₂₋₁₈ alkenylene, or C₂₋₁₈ alkynylene;
R₂ is a C₁₋₁₈ alkylene, C₂₋₁₈ alkenylene, or C₂₋₁₈ alkynylene, C₃₋₈ carbocyclylene, 3- to 8-membered heterocyclylene, or C₁₋₁₈ heteroalkylene;
R₃ and R₅ are the same or different, each independently a bond or a C₁₋₂₈ alkylene, C₂₋₂₈ alkenylene, or C₂₋₂₈ alkynylene;
R₄, R₆, and R₇ are the same or different, each independently hydrogen or a C₁₋₂₈ alkyl, C₂₋₂₈ alkenyl, or C₂₋₂₈ alkynyl, each optionally substituted by one, two, or three substituents independently selected from —OR, —SR, —S—S—R, —O—CO—R, —CO—OR, —CO—NR^{a}R^{b}, —NR^{a}—CO—R, —O—CO—NR^{a}R^{b}, —NR^{a}—CO—OR, —NR^{a}R^{b}, and —S—CO—R, wherein R at each occurrence is independently hydrogen or a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl; and R^{a} and R^{b} are independently hydrogen or lower alkyl;
X₁ is —OH, —SH, —N(R)₂, a C₅₋₈ carbocyclyl, or a heterocyclyl, hydrogen, or absent, wherein R at each occurrence is independently a lower alkyl or hydrogen;
X₂ is —O—CO—, —CO—O—, —NR—CO—, or —CO—NR—, wherein R is a lower alkyl or hydrogen; and
X₃, X₄, and X₅ are each independently —O—CO—, —CO—O—, —NR—CO—, —CO—NR—, a bond, or absent, wherein R is a lower alkyl or hydrogen.
wherein the longest chain of atoms in the compound is between 18 and 70 atoms.

In another aspect of the disclosure, the compounds described herein are of Formula III, or a salt or isomer thereof:

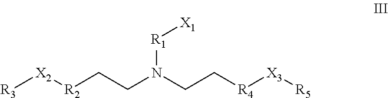

wherein:
R₁ is an optionally substituted C₁₋₁₈ alkylene, C₂₋₁₈ alkenylene, or C₂₋₁₈ alkynylene;
R₂ and R₄ are the same or different, each independently a bond or a C₁₋₂₈ alkylene, C₂₋₂₈ alkenylene, or C₂₋₂₈ alkynylene;
R₃ and R₅ are the same or different, each independently hydrogen or a C₁₋₂₈ alkyl, C₂₋₂₈ alkenyl, or C₂₋₂₈ alkynyl;
X₁ is —OH, —OR, —CO—OR, —CO—R, —O—CO—R, —SH, —SR, —N(R)₂, a C₅₋₈ carbocyclyl, a heterocyclyl, hydrogen, or absent, wherein R at each occurrence is independently hydrogen, or an optionally substituted methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl; and
X₂ and X₃ are the same or different, each independently —O—CO—, —CO—O—, —CO—, —NR—CO—, —CO—NR—, a bond, or absent, wherein R is a lower alkyl or hydrogen,
wherein the longest chain of atoms in the compound is between 18 to 70 atoms.

In another aspect of the disclosure, the compounds described herein are of Formula IV, or a salt or isomer thereof:

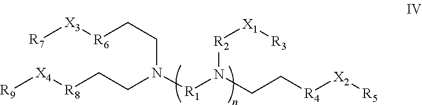

wherein:
R₁ is a C₁₋₁₈ alkylene, C₂₋₁₈ alkenylene, or C₂₋₁₈ alkynylene, each optionally substituted by one, two, or three substituents independently selected from —OR, —SR, —S—S—R, —O—CO—R, —CO—OR, —CO—NR^{a}R^{b}, —NR^{a}—CO—R, —O—CO—NR^{a}R^{b}, —NR^{a}—CO—OR, —NR^{a}R^{b}, and —S—CO—R, wherein R at each occurrence is independently hydrogen or lower alkyl; and R^{a} and R^{b} are independently hydrogen or lower alkyl;
n is 0, 1, 2, 3, 4, 5, 6, 7, 8, 9, or 10; R₂ is a C₁₋₁₈ alkylene, C₂₋₁₈ alkenylene, or C₂₋₁₈ alkynylene;
R₃ is a C₁₋₁₈ alkyl, C₂₋₁₈ alkenyl, or C₂₋₁₈ alkynyl, or a C₅₋₈ carbocyclyl, heterocyclyl, hydrogen, or absent;
R₄, R₆, and R₈ are the same or different, each independently a bond or a C₁₋₂₈ alkylene, C₂₋₂₈ alkenylene, or C₂₋₂₈ alkynylene;
R₅, R₇, and R₉ are the same or different, each independently a bond or a C₂₋₂₈ alkyl, C₂₋₂₈ alkenyl, or C₂₋₂₈ alkynyl;
X₁ is a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent; and
X₂, X₃, and X₄ are the same or different, each independently a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent,
wherein the longest chain of atoms in the compound is between 18 and 70 atoms.

In another aspect of the disclosure, the compounds described herein are of Formula V, or a salt or isomer thereof:

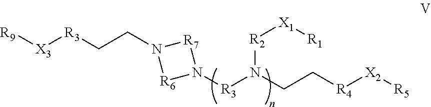

wherein:
R₁ is a C₂₋₁₈ alkyl, C₂₋₁₈ alkenyl, or C₂₋₁₈ alkynyl, a C₅₋₈ carbocyclyl, a heterocyclyl, hydrogen, or absent;
R₂ is a C₁₋₁₈ alkylene, C₂₋₁₈ alkenylene, or C₂₋₁₈ alkynylene;
R₃ is a C₁₋₁₈ alkylene, C₂₋₁₈ alkenylene, or C₂₋₁₈ alkynylene, each optionally substituted by one, two, or three substituents independently selected from —OR, —SR, —S—S—R, —O—CO—R, —CO—OR, —CO—NR^{a}R^{b}, —NR^{a}—CO—R, —O—CO—N^{a}R^{b}, —NR^{a}—CO—OR, —N^{a}R^{b}, and —S—CO—R, wherein R at each occurrence is independently hydrogen or lower alkyl; and R^{a} and R^{b} are independently hydrogen or lower alkyl;
n is 0, 1, 2, 3, 4, 5, 6, 7, 8, 9, or 10;
R₄ and R₈ are the same or different, each independently a bond or a C₁₋₂₈ alkylene, C₂₋₂₈ alkenylene, or C₂₋₂₈ alkynylene;
R₅ and R₉ are the same or different, each independently hydrogen or a C₁₋₂₈ alkyl, C₂₋₂₈ alkenyl, or C₂₋₂₈ alkynyl;
R₆ and R₇ are the same or different, each independently a bond or a C₁₋₁₈ alkylene, C₂₋₁₈ alkenylene, or C₂₋₁₈ alkynylene;
X₁ is a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent; and
X₂, and X₃ are the same or different, each independently a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent,
wherein the longest chain of atoms in the compound is between 18 to 70 atoms.

In another aspect of the disclosure, the compounds described herein are of Formula VI, or a salt or isomer thereof

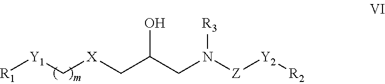

### Wherein:

Y₁ and Y₂ are the same or different, each independently selected from —(C═O)O—, —O(C═O)—, —(C═O)S—, —S(C═O)—, —NR(C═O)—, —(C═O)NR—, —O—, —S—, —S—S—, —CH₂—, an alkenylene, and an alkynylene, wherein R is independently a hydrogen, or an alkyl, alkenyl, alkynyl, carbocyclyl, heterocyclyl, group;
X is selected from —S—, —S—S—, —O—, —CH₂—, alkenylene, and alkynylene, —NR—, wherein R is hydrogen or an alkyl, alkenyl, alkynyl, carbocyclyl, heterocyclyl, group;
R₁ and R₂ are the same or different, each independently selected from carbocyclyl, alkyl, alkenyl, and alkynyl, each optionally substituted;
R₃ is selected from hydrogen, halogen, alkyl, alkenyl, alkynyl, carbocyclyl, heterocyclyl, aryl, and heteroaryl, each, except hydrogen and halogen, optionally substituted;
Z is selected from alkylene, alkenylene, alkynylene, and carbocyclylene; and
m is 1 to 24,
wherein the longest chain of atoms in the compound is between 18 to 70 atoms.

In another aspect of the invention, the compounds described herein are of Formula VII or a salt or isomer thereof

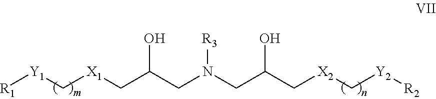

wherein:
Y₁ and Y₂ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, and alkynylene, wherein R at each occurrence is independently hydrogen, or an alkyl, alkenyl, alkynyl, carbocyclyl, heterocyclyl, group;
X₁ and X₂ are the same or different and are independently selected from —S—, —S—S—, —O—, —CH₂—, alkenylene, alkynylene, and —NR—, wherein R is hydrogen, or an alkyl, alkenyl, alkynyl, carbocyclyl, heterocyclyl, group;
R₁ and R₂ are the same or different and independently selected from alkyl, alkenyl, alkynyl, and heterocyclyl, each optionally substituted;
R₃ is selected from hydrogen, halogen, alkyl, alkenyl, alkynyl, carbocyclyl, heterocyclyl, aryl, and heteroaryl groups, each optionally substituted; and
m and n are each independently an integer selected from 1 to 24,
wherein the longest chain of atoms in the compound is between 18 to 70 atoms.

In another aspect of the invention, the compounds described herein are of Formula VIII or a salt or isomer thereof

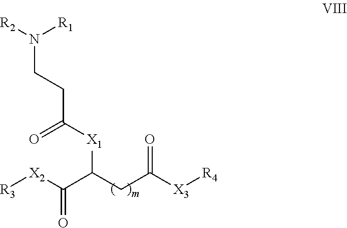

wherein:
X₁, X₂, and X₃ are independently selected from —CH₂—, —O—, —S—, and —NR—, wherein R is hydrogen or an alkyl, alkenyl, alkynyl, carbocyclyl, heterocyclyl, group;
R₁ and R₂ are the same or different and are independently selected from hydrogen, halogen, alkyl, alkenyl, alkynyl, carbocyclyl, heterocyclyl, aryl, and heteroaryl groups, each, except hydrogen and halogen, optionally substituted;
R₃ and R₄ are selected from carbocyclyl, alkyl, alkenyl, and alkynyl, each optionally substituted;
m is 0, 1, 2, 3, 4, or 5,
wherein the longest chain of atoms in the compound is between 18 and 70 atoms.

In another aspect of the invention, the compounds described herein are of Formula IX or a salt or isomer thereof

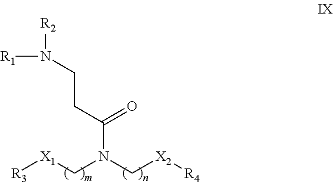

wherein:

X₁ and X₂ are the same or different moieties selected from —(C═O)O—, —O(C═O)—, —(C═O)S—, —S(C═O)—, —NR(C═O)—, —(C═O)NR—, —O—, —S—, —S—S—, —CH₂—, an alkene, and an alkyne, wherein R at each occurrence is independently hydrogen, or an alkyl, alkenyl, alkynyl, carbocyclyl, heterocyclyl group;
R₁ and R₂ are the same or different and are independently selected from hydrogen, halogen, alkyl, alkenyl, alkynyl, carbocyclyl, heterocyclyl, aryl, and heteroaryl groups, each except hydrogen and halogen optionally substituted;
R₃ and R₄ are the same or different and are independently selected from carbocyclyl, alkyl, alkenyl, and alkynyl;
m and n are the same or different and are each an integer selected from 1 to 24,
wherein the longest chain of atoms in the compound is between 18 and 70 atoms.

In another aspect of the invention, the compounds described herein are of Formula X or a salt or isomer thereof

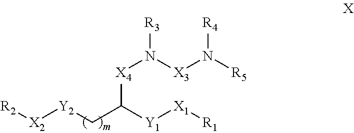

wherein:
R₁, R₂, R₃, R₄ and R₅ are the same or different and are independently selected from hydrogen, halogen, alkyl, alkenyl, alkynyl, carbocyclyl, heterocyclyl, aryl, and heteroaryl groups, each, except hydrogen and halogen, optionally substituted;
X₁, X₂, and X₃ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, and alkynylene, wherein R at each occurrence is independently hydrogen, or an alkyl, alkenyl, alkynyl, carbocyclyl, heterocyclyl, group;
X₄ is selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, and alkynylene, wherein R at each occurrence is independently hydrogen, or an alkyl, alkenyl, alkynyl, carbocyclyl, heterocyclyl, group; or X₄ is a linear alkyl group comprising 0 to 10 methylene units;
Y₁ and Y₂ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, or alkynylene, and carbocyclylene, wherein R at each occurrence is independently hydrogen, alkyl, alkenyl, alkynyl, carbocyclyl, or heterocyclyl; and
m is 0, 1, 2, 3, 4, 5, 6, 7, 8, 9, or 10,
wherein the longest chain of atoms in the compound is between 18 and 70 atoms.

In another aspect of the invention, the compounds described herein are of Formula XI or a salt or isomer thereof

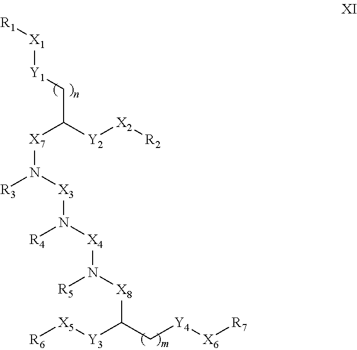

wherein:
R₁, R₂, R₃, R₄, R₅, R₆, and R₇ are the same or different and are independently selected from hydrogen, halogen, alkyl, alkenyl, alkynyl, carbocyclyl, heterocyclyl, aryl, and heteroaryl groups, each except hydrogen and halogen optionally substituted;
X₁, X₂, X₃, X₄, X₅ and X₆ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, alkynylenes, carbocyclyl, and a bond, wherein R at each occurrence is independently hydrogen, alkyl, alkenyl, alkynyl, carbocyclyl, or heterocyclyl;
X₇ and X₈ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, or alkynylene, and carbocycylyl, wherein R at each occurrence is independently hydrogen, alkyl, alkenyl, alkynyl, carbocyclyl, or heterocyclyl; or X₇ and X₈ are each independently a linear alkyl group comprising 0 to 10 methylene units;
Y₁, Y₂, Y₃, and Y₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, and an alkynylene wherein R at each occurrence is independently hydrogen, or an alkyl, alkenyl, alkynyl, carbocyclyl, heterocyclyl, group; and
n and m are independently selected from 0, 1, 2, 3, 4, 5, 6, 7, 8, 9, and 10,
wherein the longest chain of atoms in the compound is between 18 to 70 atoms.

In another aspect of the invention, the compounds described herein are of Formula XII or a salt or isomer thereof

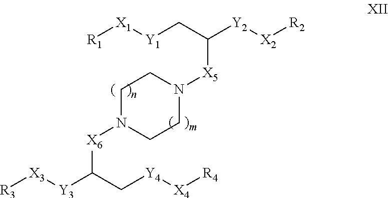

wherein:
R₁, R₂, R₃, and R₄ are the same or different and are independently selected from hydrogen, halogen, alkyl, alkenyl, alkynyl, carbocyclyl, heterocyclyl, aryl, and heteroaryl, each except hydrogen and halogen optionally substituted;
X₁, X₂, X₃, and X₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, alkynylene, and a bond, wherein R at each occurrent is independently hydrogen, alkyl, alkenyl, alkynyl, carbocyclyl, or heterocyclyl;
X₅ and X₆ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, alkynylene, and a bond, wherein R at each occurrence is independently hydrogen, or an alkyl, alkenyl, alkynyl, carbocyclyl, heterocyclyl, group; or X₅ and X₆ are each independently a linear alkyl group comprising 0 to 10 methylene units;
Y₁, Y₂, Y₃, and Y₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, alkynylene, and a bond, wherein R at each occurrence is independently hydrogen, or an alkyl, alkenyl, alkynyl, carbocyclyl, heterocyclyl, group;
the central ring structure is an optionally substituted aromatic, heteroaromatic, non-aromatic, or anti-aromatic ring system; and
n and m are independently selected from 0, 1, 2, 3, 4, 5, 6, 7, 8, 9, and 10,
wherein the longest chain of atoms in the compound is between 18 to 70 atoms.

In another aspect of the invention, the compounds described herein are of Formula XIII or a salt or isomer thereof

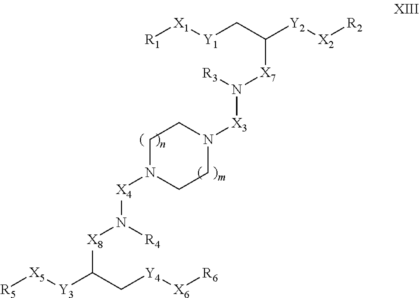

wherein:
R₁, R₂, R₃, R₄, R₅, and R₆ are the same or different and are independently selected from hydrogen, halogen, alkyl, alkenyl, alkynyl, carbocyclyl, heterocyclyl, aryl, and heteroaryl, each except hydrogen and halogen optionally substituted;
X₁, X₂, X₃, X₄, X₅, and X₆ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, alkynylene, and cyclic alkylene or heteroalkylenes, wherein R at each occurrence is independently hydrogen, or an alkyl, alkenyl, alkynyl, carbocyclyl, heterocyclyl, group;
X₇ and X₈ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, alkynylene, and cyclic alkylene or heteroalkylenes, wherein R is independently hydrogen, or an alkyl, alkenyl, alkynyl, carbocyclyl, heterocyclyl, group; or X₇ and X₈ are each a linear alkyl group comprising 0 to 10 methylene units;
Y₁, Y₂, Y₃, and Y₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, alkynylenes, and cyclic alkylene or heteroalkylenes, wherein R at each occurrence is independently hydrogen, or an alkyl, alkenyl, alkynyl, carbocyclyl, heterocyclyl, group;
the central ring structure is an optionally substituted aromatic, heteroaromatic, non-aromatic, or anti-aromatic ring system; and
n and m are independently selected from 0, 1, 2, 3, 4, 5, 6, 7, 8, 9, and 10,
wherein the longest chain of atoms in the compound is between 18 to 70 atoms.

In another aspect of the invention, the compounds described herein are of Formula XIV or a salt or isomer thereof

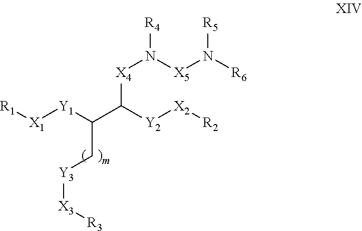

wherein:
R₁, R₂, R₃, R₄, R₅, and R₆ the same or different and are independently selected from hydrogen, halogen, alkyl, alkenyl, alkynyl, carbocyclyl, heterocyclyl, aryl, and heteroaryl, each except hydrogen and halogen optionally substituted;
X₁, X₂, X₃, X₄, and X₅ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, alkynylene, cycloalkylene, and heterocyclylene, wherein R is independently hydrogen, alkyl, alkenyl, alkynyl, carbocyclyl, and heterocyclyl;
Y₁, Y₂, and Y₃ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkylene, alkenylene, alkynylene, cycloalkylene, and heterocyclylene, wherein R is independently hydrogen, alkyl, alkenyl, alkynyl, carbocyclyl, or heterocyclyl; and
m is selected from 0, 1, 2, 3, 4, 5, 6, 7, 8, 9, and 10,
wherein the longest chain of atoms in the compound is between 18 to 70 atoms.

In another aspect, the present disclosure provides methods of synthesis and characterization of compounds of Formula I, II, III, IV, V, VI, VII, VIII, IX, X, XI, XII, XIII, and XIV and methods of making a nanoparticle composition comprising a lipid component such as a compound of Formula I, II, III, IV, V, VI, VII, VIII, IX, X, XI, XII, XIII, or XIV and a biological or therapeutic agent, such as, but not limited to, a nucleic acid (e.g., mRNA).

Examples of biologically active and/or therapeutic molecules include but are not limited to: (1) polynucleotides such as mRNA, rRNA, RNAi, microRNA, plasmids, aptamers, DNA, cDNA; (2) antisense polynucleotides; (3) low molecular weight compounds (synthetic or naturally occurring) such as peptides, hormones, and antibiotics; and (4) proteins.

In some embodiments, the active agent is fully encapsulated within the lipid nanoparticle composition such that the active agent is resistant to enzymatic degradation (e.g., by a nuclease). In other some embodiments, the lipid particles are non-toxic to mammals (e.g., humans).

In one embodiment, the lipid nanoparticle composition may comprise one or more ionizable lipids, neutral lipids, PEG-modified lipids, or cholesterol. For example, the lipid nanoparticle composition may comprise at least one of the following ionizable lipids: DLin-MC3-DMA, DODMA, DODAP, SM-102, ALC-0315, C12-200, or one of the ionizable lipids described in Formula I, II, III, IV, V, VI, VII, VIII, IX, X, XI, XII, XIII, or XIV. In some embodiments, the lipid nanoparticle compositions comprise PEG₂₀₀₀-DMG or other PEG conjugated lipid. In some embodiments, the lipid nanoparticle compositions comprise cholesterol. In certain embodiments, the lipid nanoparticle composition comprises at least one neutral lipid (e.g., DSPE, DOPE, DSPC, HSPC).

In another aspect, the present disclosure provides lipid nanoparticle compositions comprising (a) one or more active agent; (b) one or more ionizable lipid comprising from about 10 mol % to about 85 mol % of the total lipid present in the composition; (c) one or more neutral “helper” lipids comprising from about 5 mol % to about 40 mol % of the total lipid present in the composition; (d) one or more PEG-conjugated lipids that inhibit aggregation of particles comprising from about 0 mol % to about 10 mol % of the total lipid present in the composition; and (e) cholesterol comprising from about 10 mol % to 50 mol % of the total lipid present in the composition.

The present disclosure also provides the compositions and methods useful for facilitating the transfection and delivery of one or more nucleic acid molecule to cells. In some embodiments, the secreted protein is produced for sustained amounts of time. For example, the secreted protein may be produced for more than one hour, more than three hours, more than 6 hours, more than 10 hours, more than 24 hours, more than 48 hours, or more than 72 hours after administration. In some embodiments, the protein expression is sustained at least at therapeutic levels.

In another aspect, this disclosure features a lipid nanoparticle composition according to preceding aspects and an acceptable pharmaceutically relevant carrier. For example, the lipid nanoparticle composition may be suspended in a buffer or other solution designed to facilitate stability during storage and/or shipment. In some embodiments, the lipid nanoparticle composition may be refrigerated (e.g., being stored at a temperature of about 2 to 8° C.). In other embodiments, the lipid nanoparticle composition may be frozen (e.g., temperatures below 0° C. (e.g., about −5° C., −10° C., −15° C., −20° C., −30° C., −40° C., −50° C., −60° C., −70° C., −80° C., −90° C., −100° C.-120° C., −140° C., or −160° C.)). In other embodiments, the lipid nanoparticle composition may be lyophilized in the presence of sucrose, lactose, or other saccharides or excipients (e.g., bulking agents, collapse temperature modifiers, amino acids, polyols, buffering agents, complexing agents, tonicity modifiers, or antioxidants). The lyophilized lipid nanoparticle composition cake can be stored preferably in a sterile lyophilization vial and later rehydrated with sterile water for injection.

In another aspect, the present disclosure provides a method for treatment of a disease or condition in a subject in need of treatment, the method comprising administering to the subject a therapeutically effective amount of a therapeutic agent through delivery by a lipid nanoparticle composition according to any aspects or embodiments disclosed herein.

In another aspect, the present disclosure provides a method for delivery of a therapeutic agent to a subject for treatment of a disease or condition in a subject in need of treatment using a lipid nanoparticle composition according to any aspects or embodiments disclosed.

In another aspect, the present disclosure provides use of a lipid nanoparticle composition (LNP) according to any aspects or embodiments disclosed in the manufacture of a medicament for treatment of a disease or condition in a subject in need of treatment.

Such disease or condition may be any diseases or disorders that are treatable by a therapeutic agent that can be delivered by the LNP to the subject in need of treatment.

Other aspects or advantages of the present disclosure will be better understood by a person of skill in the pertinent art in view of the following drawings, detailed description, examples, and claims.

### BRIEF DESCRIPTION OF THE DRAWINGS

FIG. 1 illustrates representative dynamic light scattering (DLS) profiles of novel lipid nanoparticle compositions.

FIG. 2 illustrates additional representative dynamic light scattering (DLS) profiles of novel lipid nanoparticle compositions.

FIG. 3 illustrates additional representative dynamic light scattering (DLS) profiles of novel lipid nanoparticle compositions.

FIG. 4 illustrates additional representative dynamic light scattering (DLS) profiles of novel lipid nanoparticle compositions.

FIG. 5 illustrates the representative in vitro transfection efficiency of novel lipid nanoparticle compositions relative to a naked mRNA standard.

FIG. 6 illustrates additional representative in vitro transfection efficiency of novel lipid nanoparticle compositions relative to a naked mRNA standard.

FIG. 7 illustrates additional representative in vitro transfection efficiency of novel lipid nanoparticle compositions relative to a naked mRNA standard.

### DETAILED DESCRIPTION OF THE DISCLOSURE

This disclosure relates to novel lipids and lipid nanoparticle compositions containing a novel lipid. This disclosure also provides methods of delivering a biological and/or therapeutic agent to a mammalian cell or organ and treating a disease or disorder using a lipid nanoparticle composition (LNP). A method of delivering a biological and/or therapeutic agent to a mammalian cell or organ may involve administration of a nanoparticle composition containing the biological and/or therapeutic agent to a subject, in which the cell or organ makes contact with the composition, whereby the biological and/or therapeutic agent is delivered to the cell or organ.

### I. Ionizable Lipids

The present disclosure provides lipids comprising an amine moiety and one or more biodegradable groups. The lipids described herein may be used in nanoparticle compositions for the delivery of biological and/or therapeutic agents to mammalian cells or organs.

In one aspect of the disclosure, the compounds described herein are of Formula I or a salt or isomer thereof

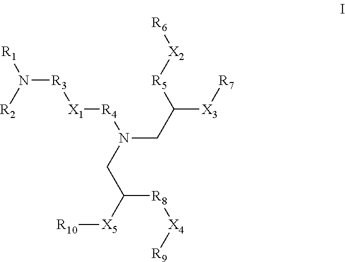

wherein:
R₁ and R₂ are the same or different, each independently alkyl, alkenyl, or alkynyl; or alternatively R₁ and R₂ together with the nitrogen atom to which they are attached form a heterocyclyl;
R₃ is a C₁₋₁₈ alkylene, C₂₋₁₈ alkenylene, or C₂₋₁₈ alkynylene, or together with the adjacent nitrogen atom forms a ring structure comprising 3-18 carbon atoms;
R₄ is a C₁₋₁₈ alkylene, C₂₋₁₈ alkenylene, or C₂₋₁₈ alkynylene;
R₅ and R₈ are the same or different, each independently a bond, or a C₁₋₂₈ alkylene, C₂₋₂₈ alkenylene, or C₂₋₂₈ alkynylene;
R₆ and R₉ are the same or different, each independently hydrogen or a linear C₁₋₂₈ alkyl or C₂₋₂₈ alkenyl;
R₇ and R₁₀ are the same or different, each independently hydrogen or a linear C₁₋₂₈ alkyl or C₂₋₂₈ alkenyl;
X₁ is a bond, —O—, —CO—, —OC—O—, or —O—CO—;
X₂ and X₄ are the same or different, each independently methylene (—CH₂—), —S—, —S—S—, —O—, —O—CO—, —CO—O—, or —NR—, wherein R is a lower alkyl; and
X₃ and X₅ are the same or different, each independently a methylene (—CH₂—), —S—, —S—S—, —O—, —O—CO—, —CO—O—, or —NR—, wherein R is a lower alkyl,
wherein the longest chain of atoms in the compound is between 18 to 70 atoms.

In some embodiments, R₁ and R₂, together with the atom to which they are attached, form a part of a 5- to 8-membered aromatic or non-aromatic heterocycle having one or more heteroatoms selected from N, O, S, and P. In certain embodiments, the heterocycle formed by R₁ and R₂ is substituted with one or more C₃₋₇ carbocycle or 3- to 8-membered heterocycle groups or other functional groups, such as alkyl, alkenyl, alkynyl, —OH, —SH, —OR, —SR, —NR₂, -oxo, or combinations thereof.

In some embodiments, R₃, X₁, and R₄ may form a part of a 3- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₅, X₂, and X₃ may together with the atom they are attached form a part of a 4- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₈, X₄, and X₅ may together with the atom they are attached form a part of a 4- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₁ and R₂ are each independently selected from methyl, ethyl, and isopropyl; or alternatively R₁ and R₂ together with the nitrogen atom to which they are attached form a 5- or 6-membered heterocyclyl.

In some embodiments, R₃ is a linear alkyl comprising at least 3 carbons.

In some embodiments, in the compound of Formula I:
R₁ and R₂ are each independently methyl, ethyl, propyl, isopropyl, butyl, or isobutyl; or alternatively R₁ and R₂ together with the nitrogen atom to which they are attached form a heterocyclyl;
R₃ is a C₁₋₈ alkylene, C₂₋₈ alkenylene, or C₂₋₈ alkenylene;
R₄ is a C₁₋₈ alkylene, C₂₋₈ alkenylene, or C₂₋₈ alkenylene;
R₅ and R₈ the same or different, each independently a bond, C₁₋₁₄ alkylene, C₂₋₁₄ alkenylene, or C₂₋₁₄ alkynylene;
R₆ and R₉ are the same or different, each independently hydrogen, linear C₁₋₁₄ alkyl, or C₂₋₁₄ alkenyl;
R₇ and R₁₀ are the same or different, each independently hydrogen, linear C₁₋₁₄ alkyl, or C₂₋₁₄ alkenyl;
X₁ is a bond, —CO—, —OC—O—, or —O—CO—;
X₂ and X₄ are the same or different, each independently methylene (—CH₂—), —O—, —O—CO—, or —CO—O—; and
X₃ and X₅ are the same or different, each independently methylene (—CH₂—), —O—, —O—CO—, or —CO—O—.

In some embodiments, the compound of Formula I may be selected from the Compounds of List 1:

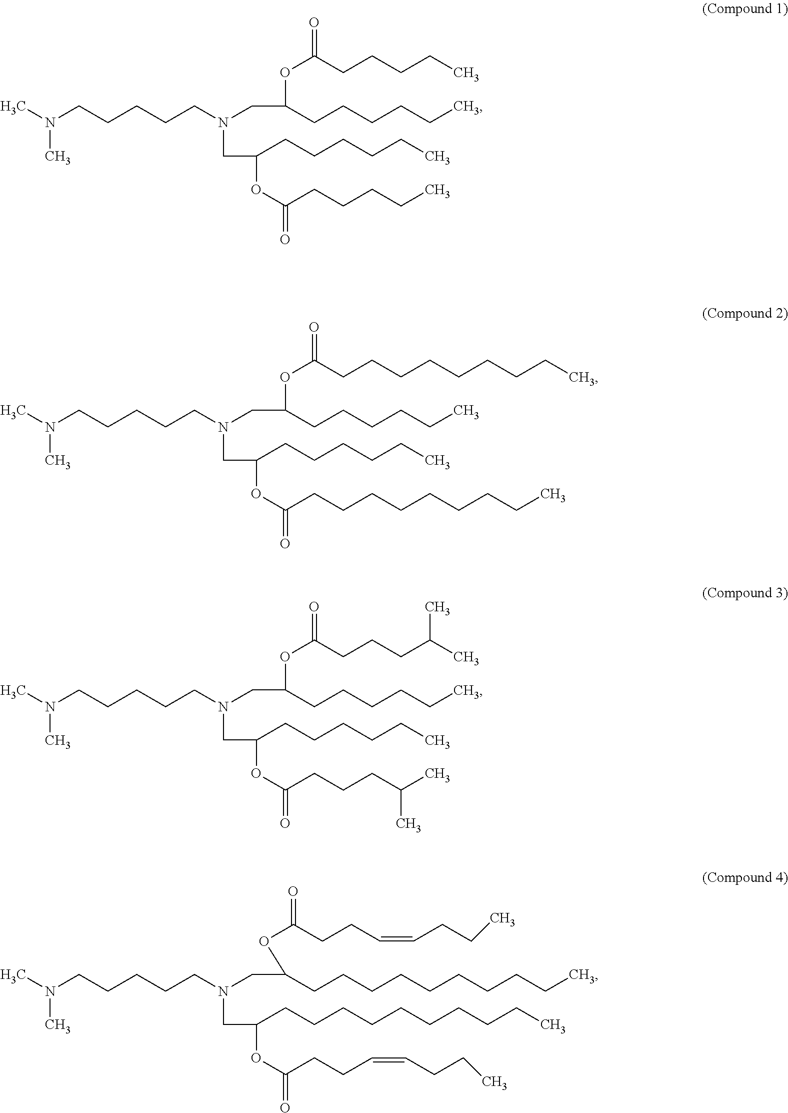

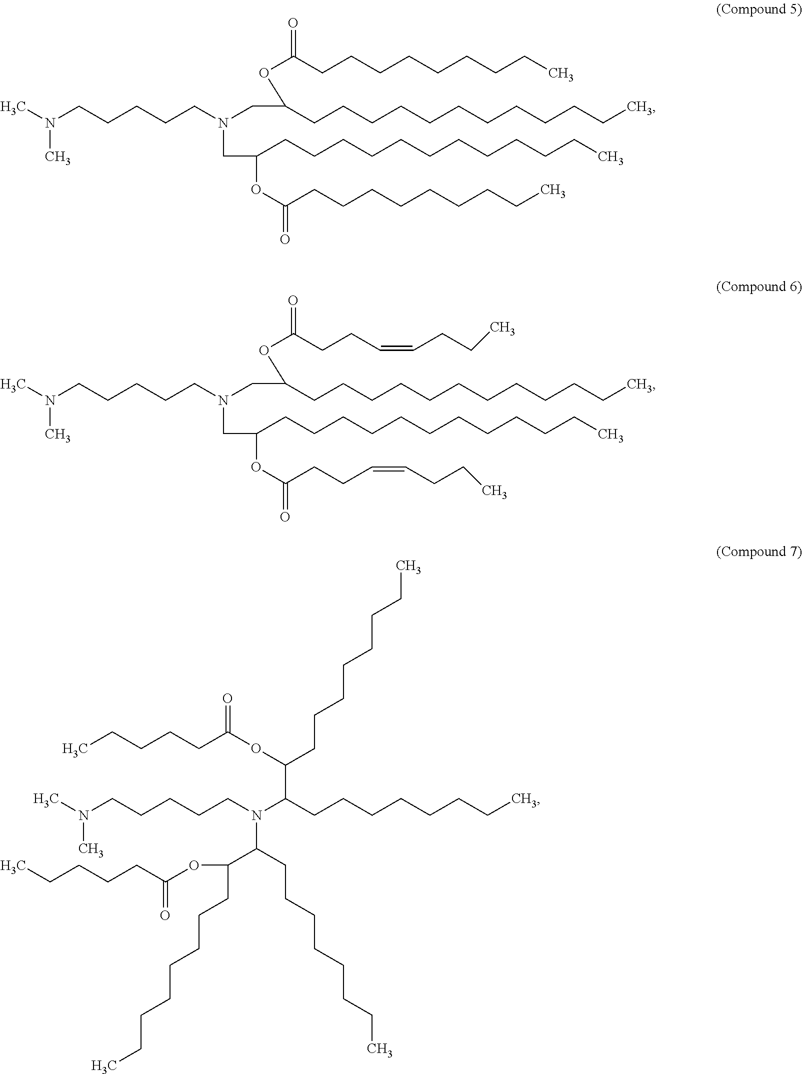 [SMILES: CCCCCCCCCCCCC(CN(CCCCCN(C)C)CC(CCCCCCCCCCCC)OC(=O)CCCCCCCCC)OC(=O)CCCCCCCCC ; CCC/C=C\CCC(=O)OC(CCCCCCCCCCCC)CN(CCCCCN(C)C)CC(CCCCCCCCCCCC)OC(=O)CC/C=C\CCC ; CCCCCCCCC(OC(=O)CCCCC)C(CCCCCCCC)N(CCCCCN(C)C)C(CCCCCCCC)C(CCCCCCCC)OC(=O)CCCCC]

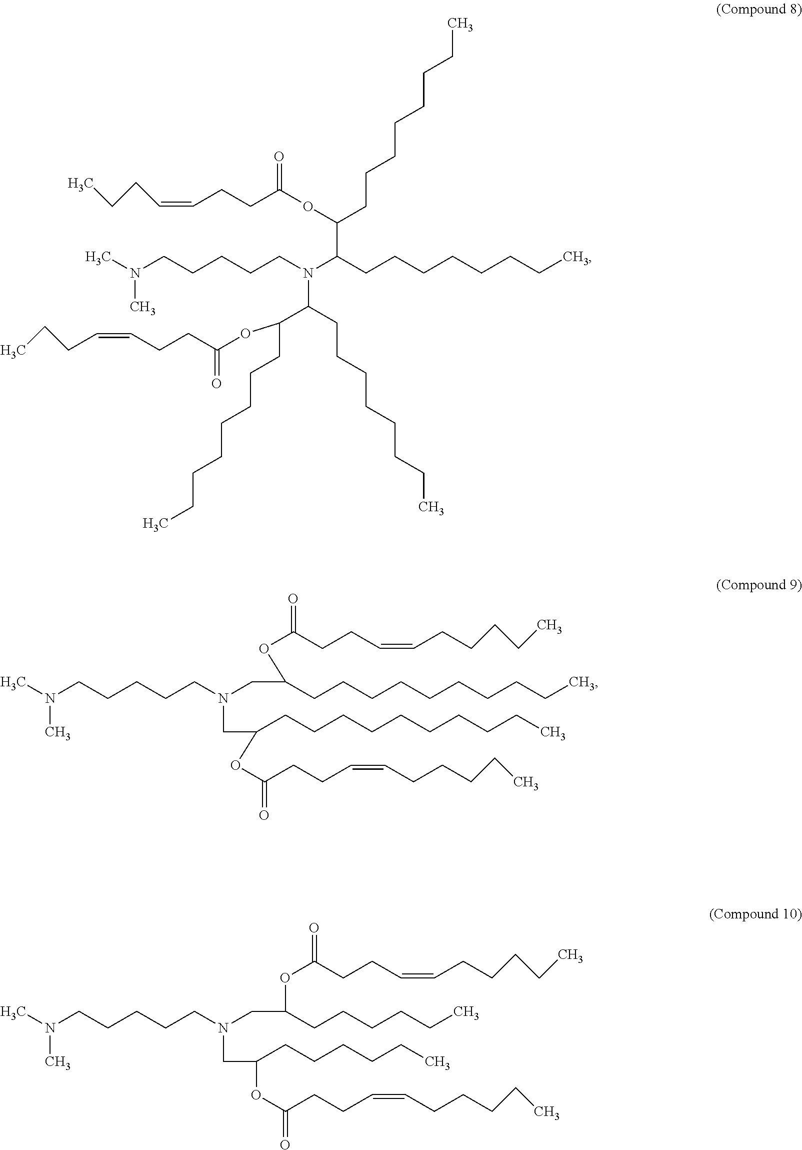 [SMILES: CCC/C=C\CCC(=O)OC(CCCCCCCC)C(CCCCCCCC)N(CCCCCN(C)C)C(CCCCCCCC)C(CCCCCCCC)OC(=O)CC/C=C\CCC ; CCCCC/C=C\CCC(=O)OC(CCCCCCCCCC)CN(CCCCCN(C)C)CC(CCCCCCCCCC)OC(=O)CC/C=C\CCCCC ; CCCCC/C=C\CCC(=O)OC(CCCCCC)CN(CCCCCN(C)C)CC(CCCCCC)OC(=O)CC/C=C\CCCCC]

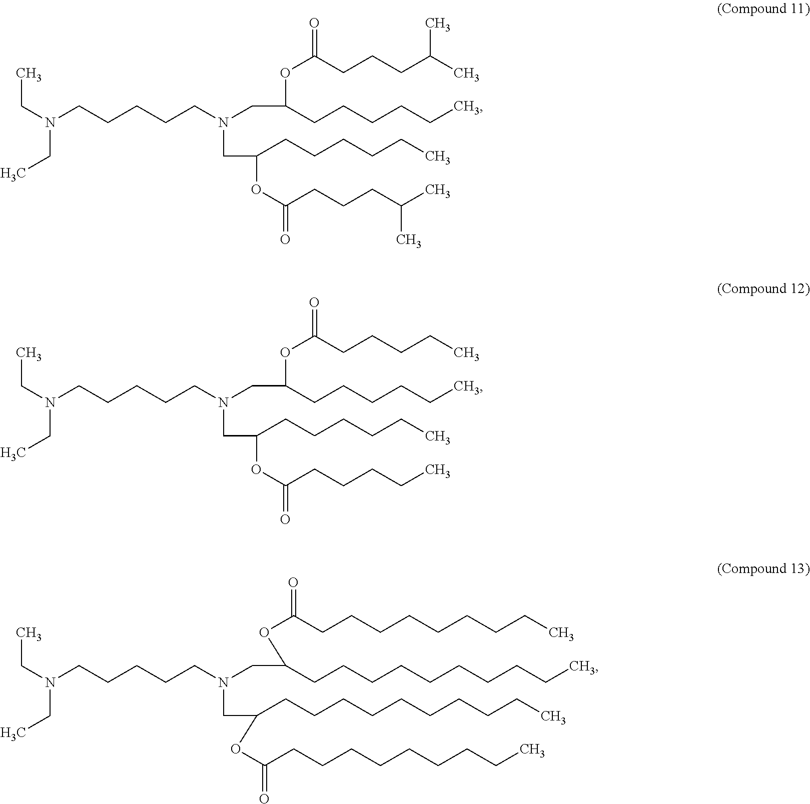 [SMILES: CCCCCCC(CN(CCCCCN(CC)CC)CC(CCCCCC)OC(=O)CCCC(C)C)OC(=O)CCCC(C)C ; CCCCCCC(CN(CCCCCN(CC)CC)CC(CCCCCC)OC(=O)CCCCC)OC(=O)CCCCC ; CCCCCCCCCCC(CN(CCCCCN(CC)CC)CC(CCCCCCCCCC)OC(=O)CCCCCCCCC)OC(=O)CCCCCCCCC]

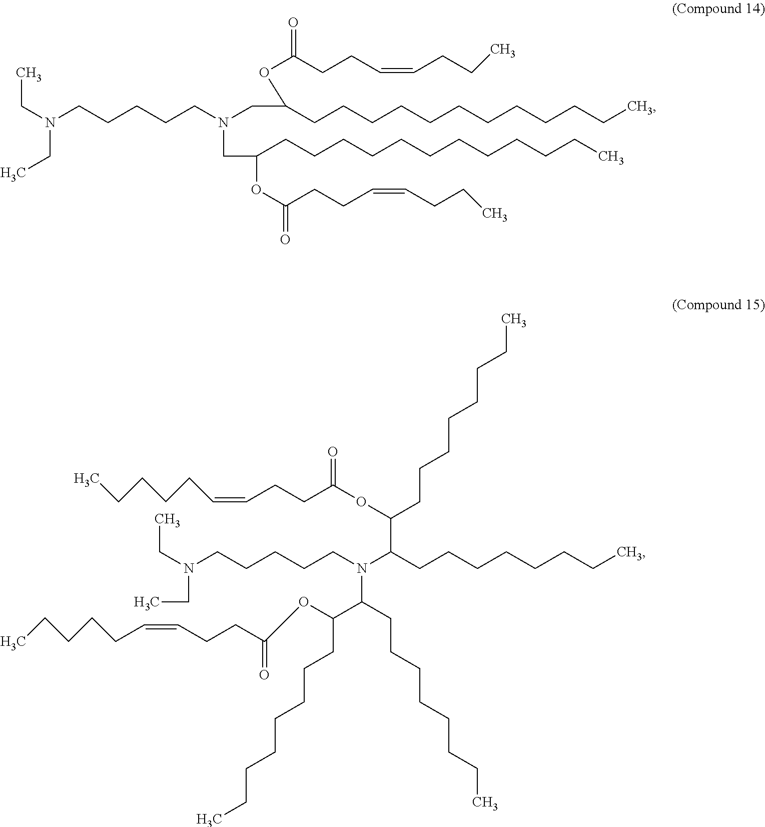 [SMILES: CCC/C=C\CCC(=O)OC(CCCCCCCCCCCC)CN(CCCCCN(CC)CC)CC(CCCCCCCCCCCC)OC(=O)CC/C=C\CCC ; CCCCC/C=C\CCC(=O)OC(CCCCCCCC)C(CCCCCCCC)N(CCCCCN(CC)CC)C(CCCCCCCC)C(CCCCCCCC)OC(=O)CC/C=C\CCCCC]

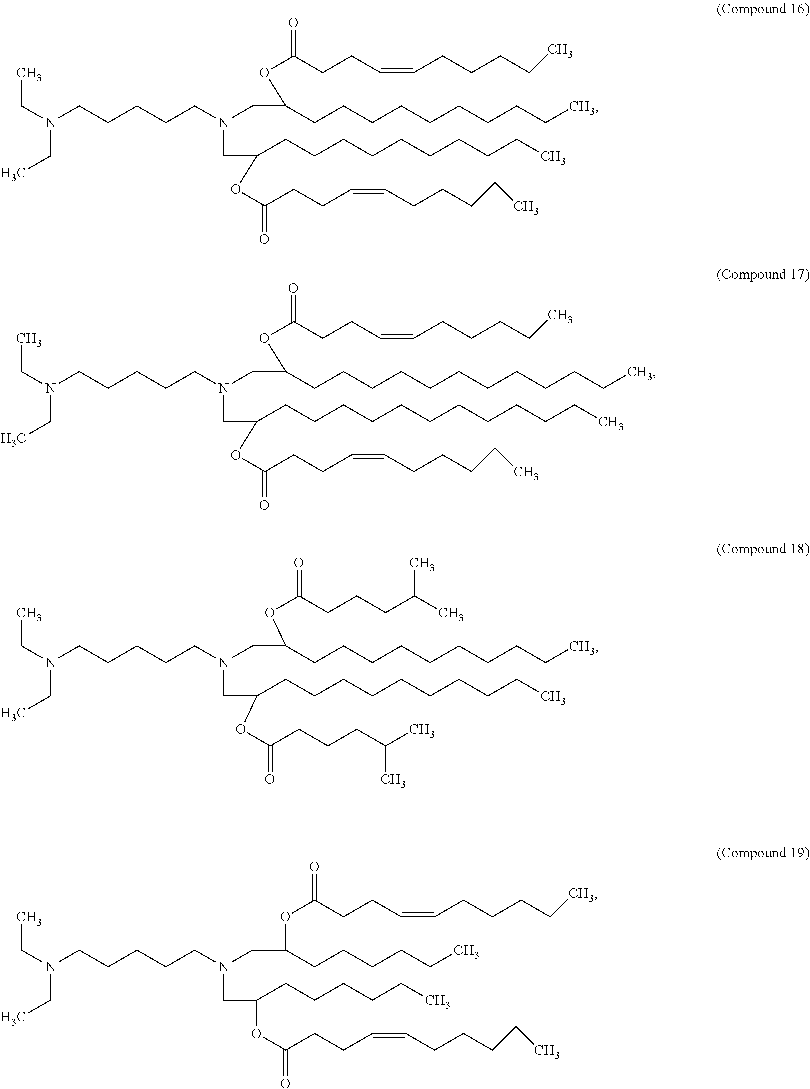 [SMILES: CCCCC/C=C\CCC(=O)OC(CCCCCCCCCC)CN(CCCCCN(CC)CC)CC(CCCCCCCCCC)OC(=O)CC/C=C\CCCCC ; CCCCC/C=C\CCC(=O)OC(CCCCCCCCCCCC)CN(CCCCCN(CC)CC)CC(CCCCCCCCCCCC)OC(=O)CC/C=C\CCCCC ; CCCCCCCCCCC(CN(CCCCCN(CC)CC)CC(CCCCCCCCCC)OC(=O)CCCC(C)C)OC(=O)CCCC(C)C ; CCCCC/C=C\CCC(=O)OC(CCCCCC)CN(CCCCCN(CC)CC)CC(CCCCCC)OC(=O)CC/C=C\CCCCC]

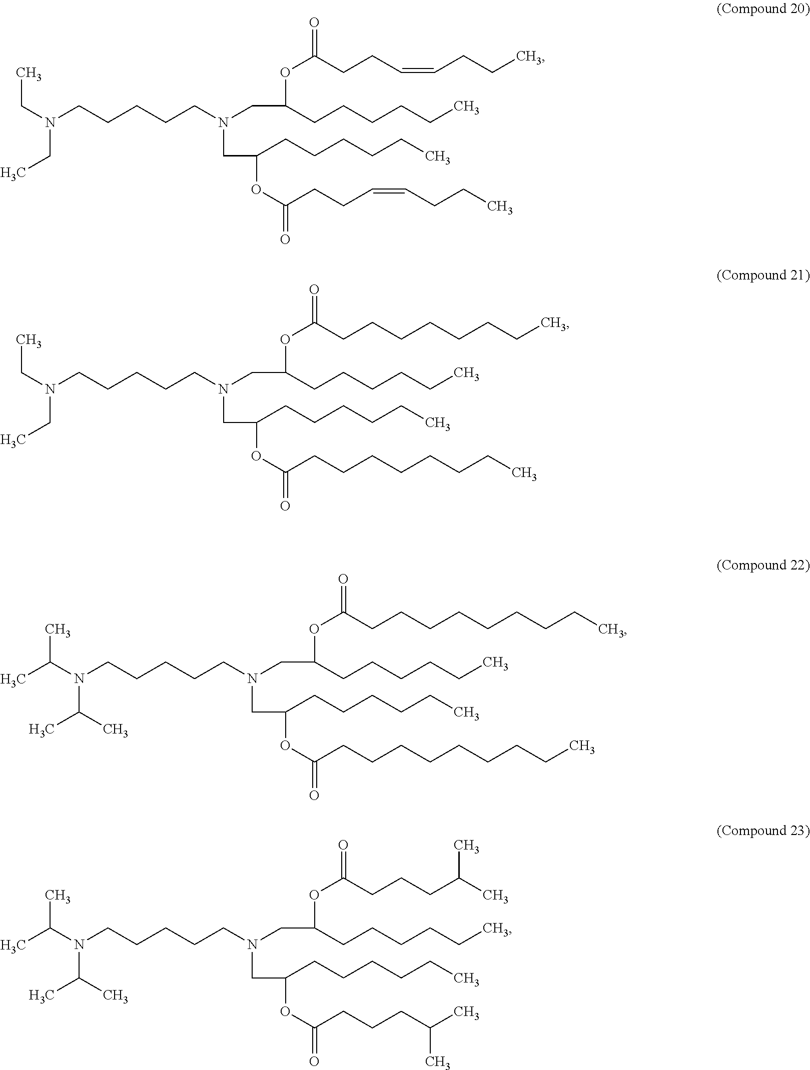 [SMILES: CCC/C=C\CCC(=O)OC(CCCCCC)CN(CCCCCN(CC)CC)CC(CCCCCC)OC(=O)CC/C=C\CCC ; CCCCCCCCC(=O)OC(CCCCCC)CN(CCCCCN(CC)CC)CC(CCCCCC)OC(=O)CCCCCCCC ; CCCCCCCCCC(=O)OC(CCCCCC)CN(CCCCCN(C(C)C)C(C)C)CC(CCCCCC)OC(=O)CCCCCCCCC ; CCCCCCC(CN(CCCCCN(C(C)C)C(C)C)CC(CCCCCC)OC(=O)CCCC(C)C)OC(=O)CCCC(C)C]

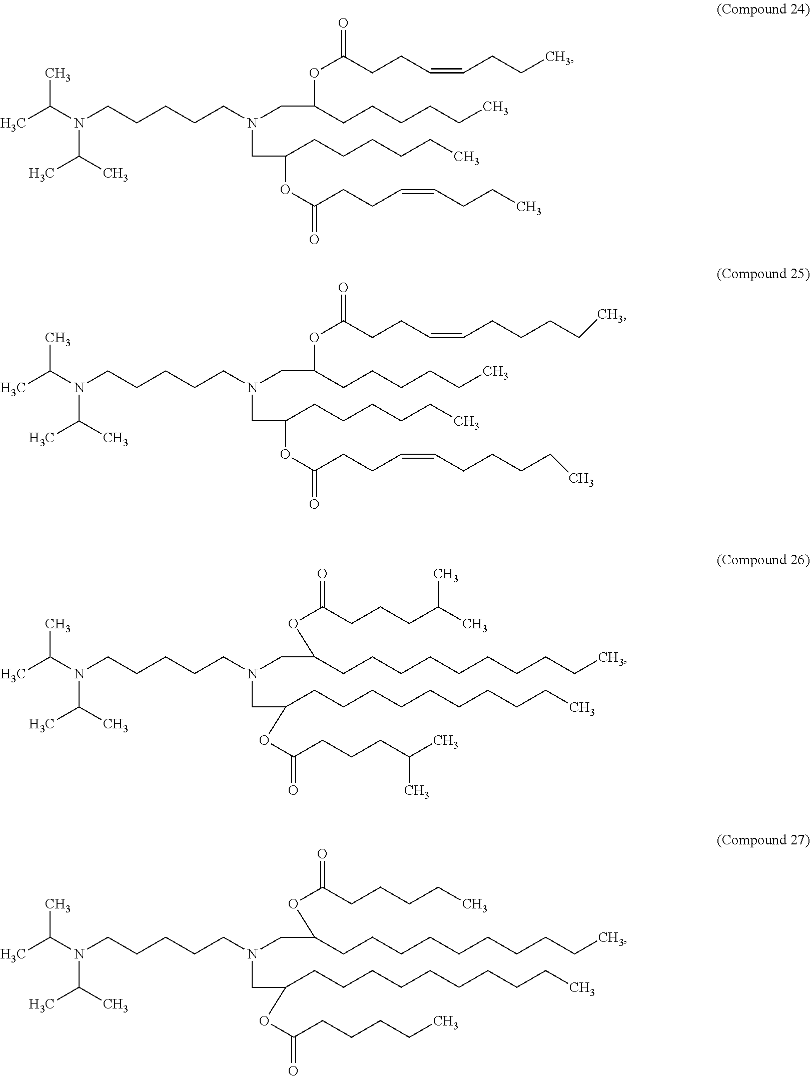 [SMILES: CCC/C=C\CCC(=O)OC(CCCCCC)CN(CCCCCN(C(C)C)C(C)C)CC(CCCCCC)OC(=O)CC/C=C\CCC ; CCCCC/C=C\CCC(=O)OC(CCCCCC)CN(CCCCCN(C(C)C)C(C)C)CC(CCCCCC)OC(=O)CC/C=C\CCCCC ; CCCCCCCCCCC(CN(CCCCCN(C(C)C)C(C)C)CC(CCCCCCCCCC)OC(=O)CCCC(C)C)OC(=O)CCCC(C)C ; CCCCCCCCCCC(CN(CCCCCN(C(C)C)C(C)C)CC(CCCCCCCCCC)OC(=O)CCCCC)OC(=O)CCCCC]

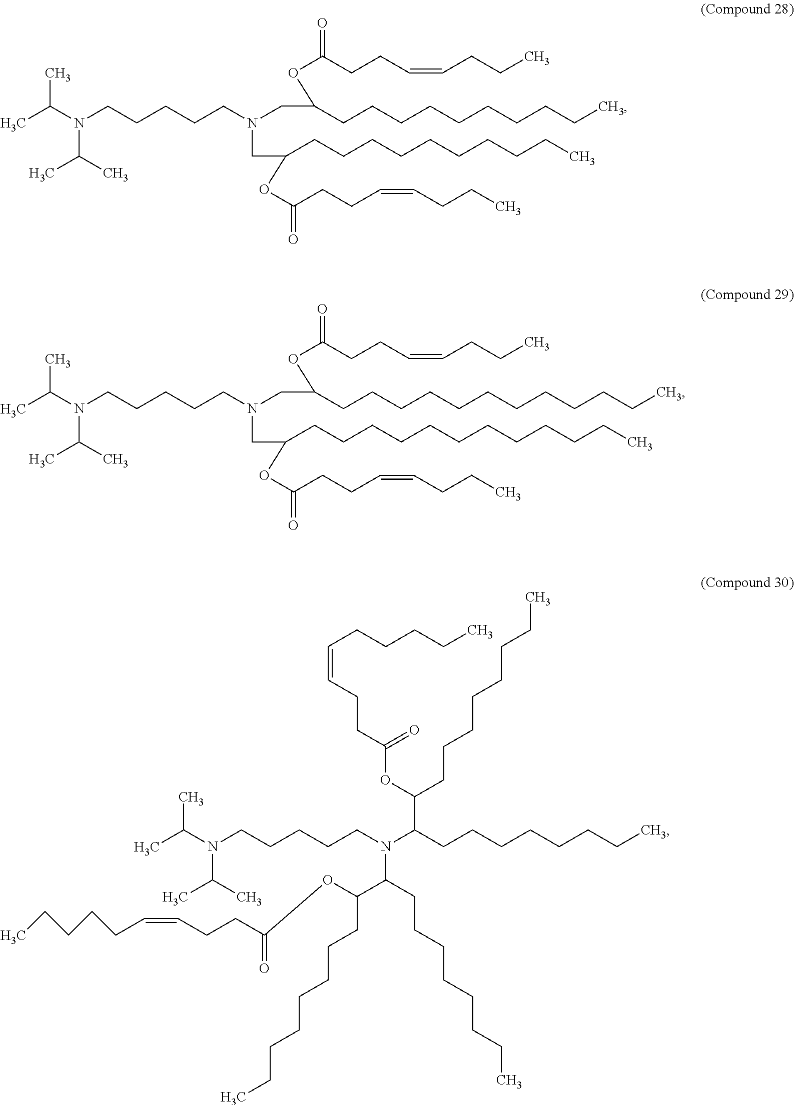 [SMILES: CCC/C=C\CCC(=O)OC(CCCCCCCCCC)CN(CCCCCN(C(C)C)C(C)C)CC(CCCCCCCCCC)OC(=O)CC/C=C\CCC ; CCC/C=C\CCC(=O)OC(CCCCCCCCCCCC)CN(CCCCCN(C(C)C)C(C)C)CC(CCCCCCCCCCCC)OC(=O)CC/C=C\CCC ; CCCCC/C=C\CCC(=O)OC(CCCCCCCC)C(CCCCCCCC)N(CCCCCN(C(C)C)C(C)C)C(CCCCCCCC)C(CCCCCCCC)OC(=O)CC/C=C\CCCCC]

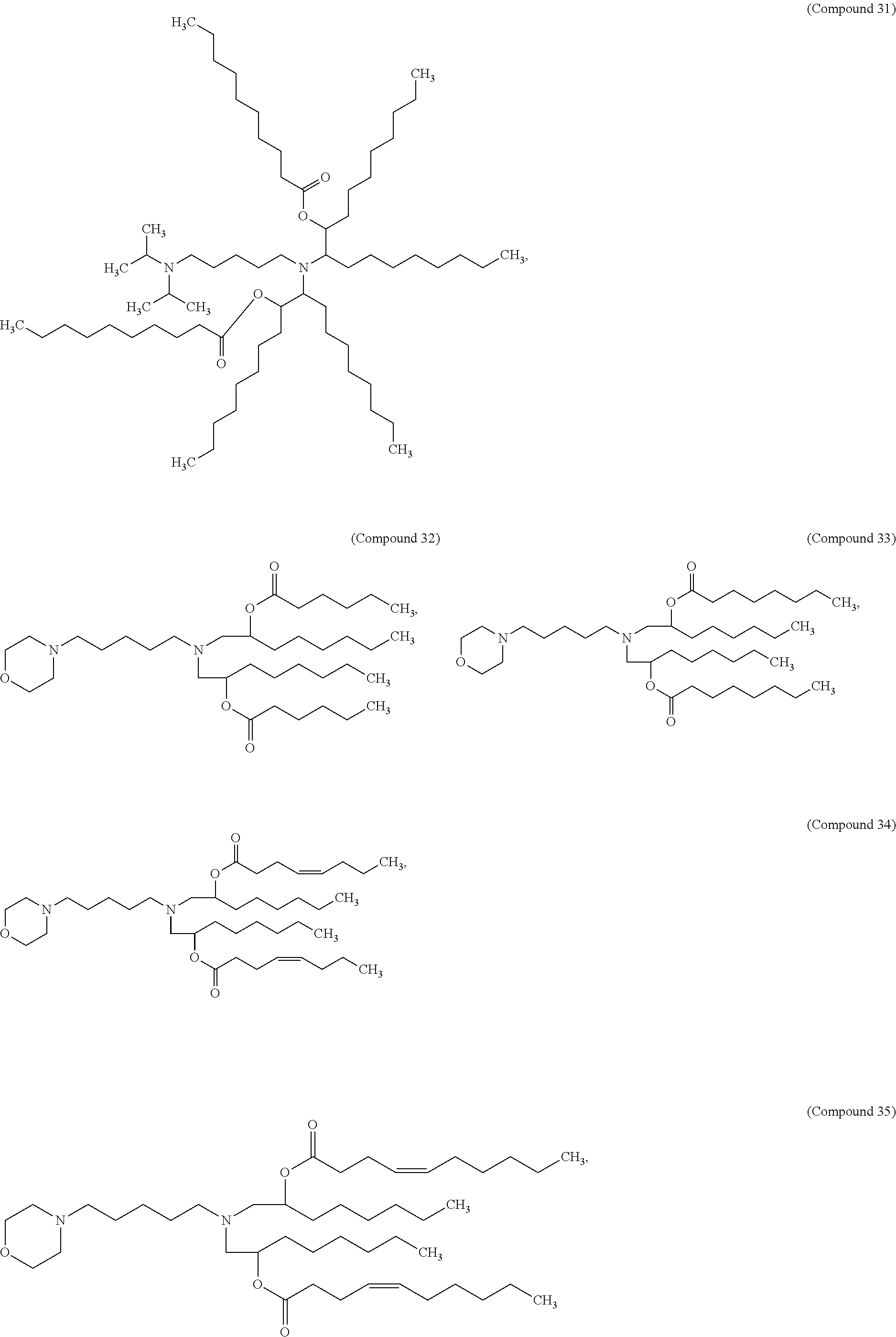 [SMILES: CCCCCCCCCC(=O)OC(CCCCCCCC)C(CCCCCCCC)N(CCCCCN(C(C)C)C(C)C)C(CCCCCCCC)C(CCCCCCCC)OC(=O)CCCCCCCCC ; CCCCCCC(CN(CCCCCN1CCOCC1)CC(CCCCCC)OC(=O)CCCCC)OC(=O)CCCCC ; CCCCCCCC(=O)OC(CCCCCC)CN(CCCCCN1CCOCC1)CC(CCCCCC)OC(=O)CCCCCCC ; CCC/C=C\CCC(=O)OC(CCCCCC)CN(CCCCCN1CCOCC1)CC(CCCCCC)OC(=O)CC/C=C\CCC ; CCCCC/C=C\CCC(=O)OC(CCCCCC)CN(CCCCCN1CCOCC1)CC(CCCCCC)OC(=O)CC/C=C\CCCCC]

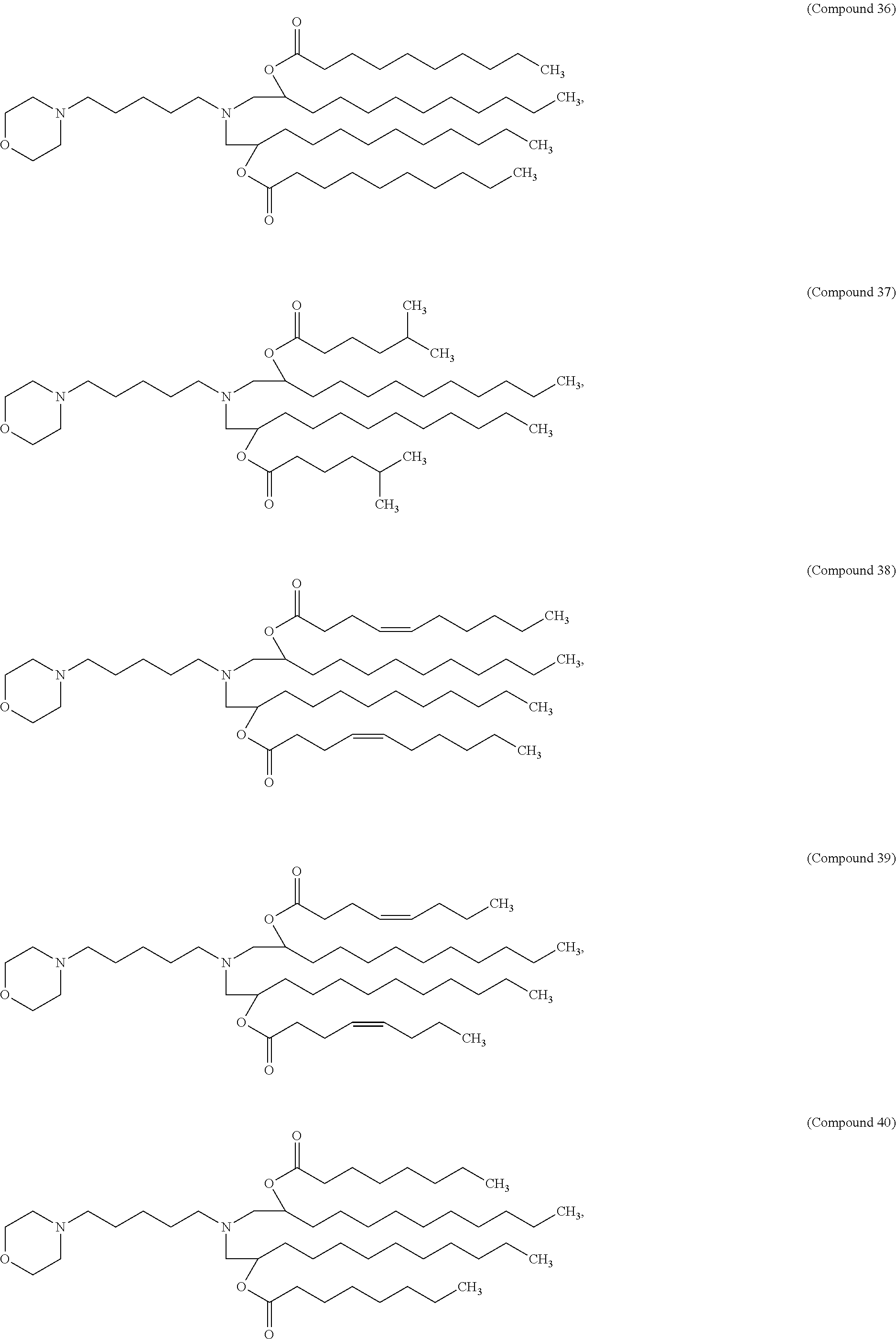 [SMILES: CCCCCCCCCCC(CN(CCCCCN1CCOCC1)CC(CCCCCCCCCC)OC(=O)CCCCCCCCC)OC(=O)CCCCCCCCC ; CCCCCCCCCCC(CN(CCCCCN1CCOCC1)CC(CCCCCCCCCC)OC(=O)CCCC(C)C)OC(=O)CCCC(C)C ; CCCCC/C=C\CCC(=O)OC(CCCCCCCCCC)CN(CCCCCN1CCOCC1)CC(CCCCCCCCCC)OC(=O)CC/C=C\CCCCC ; CCC/C=C\CCC(=O)OC(CCCCCCCCCC)CN(CCCCCN1CCOCC1)CC(CCCCCCCCCC)OC(=O)CC/C=C\CCC ; CCCCCCCCCCC(CN(CCCCCN1CCOCC1)CC(CCCCCCCCCC)OC(=O)CCCCCCC)OC(=O)CCCCCCC]

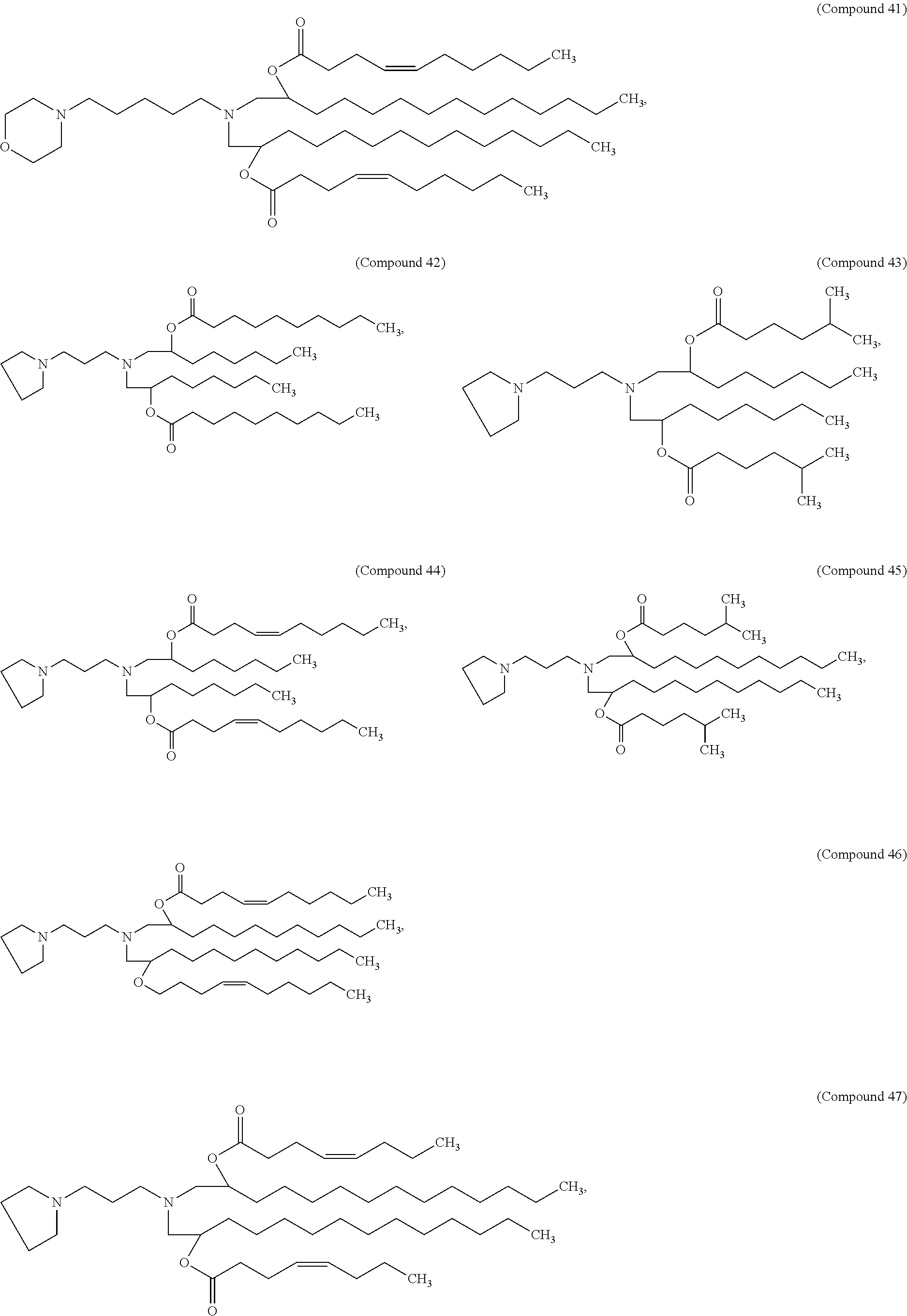

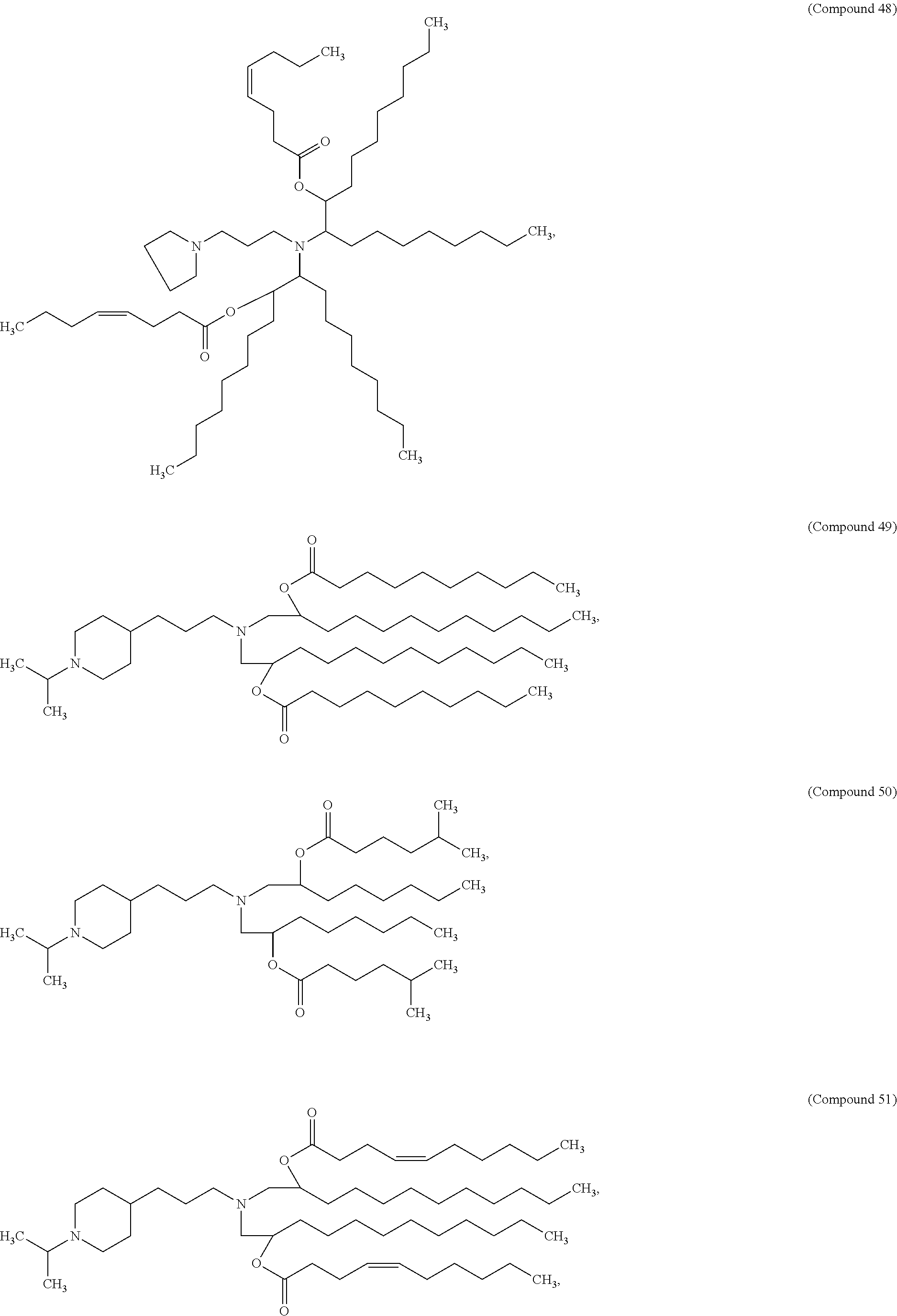 [SMILES: CCC/C=C\CCC(=O)OC(CCCCCCCC)C(CCCCCCCC)N(CCCN1CCCC1)C(CCCCCCCC)C(CCCCCCCC)OC(=O)CC/C=C\CCC ; CCCCCCCCCCC(CN(CCCC1CCN(C(C)C)CC1)CC(CCCCCCCCCC)OC(=O)CCCCCCCCC)OC(=O)CCCCCCCCC ; CCCCCCC(CN(CCCC1CCN(C(C)C)CC1)CC(CCCCCC)OC(=O)CCCC(C)C)OC(=O)CCCC(C)C ; CCCCC/C=C\CCC(=O)OC(CCCCCCCCCC)CN(CCCC1CCN(C(C)C)CC1)CC(CCCCCCCCCC)OC(=O)CC/C=C\CCCCC]

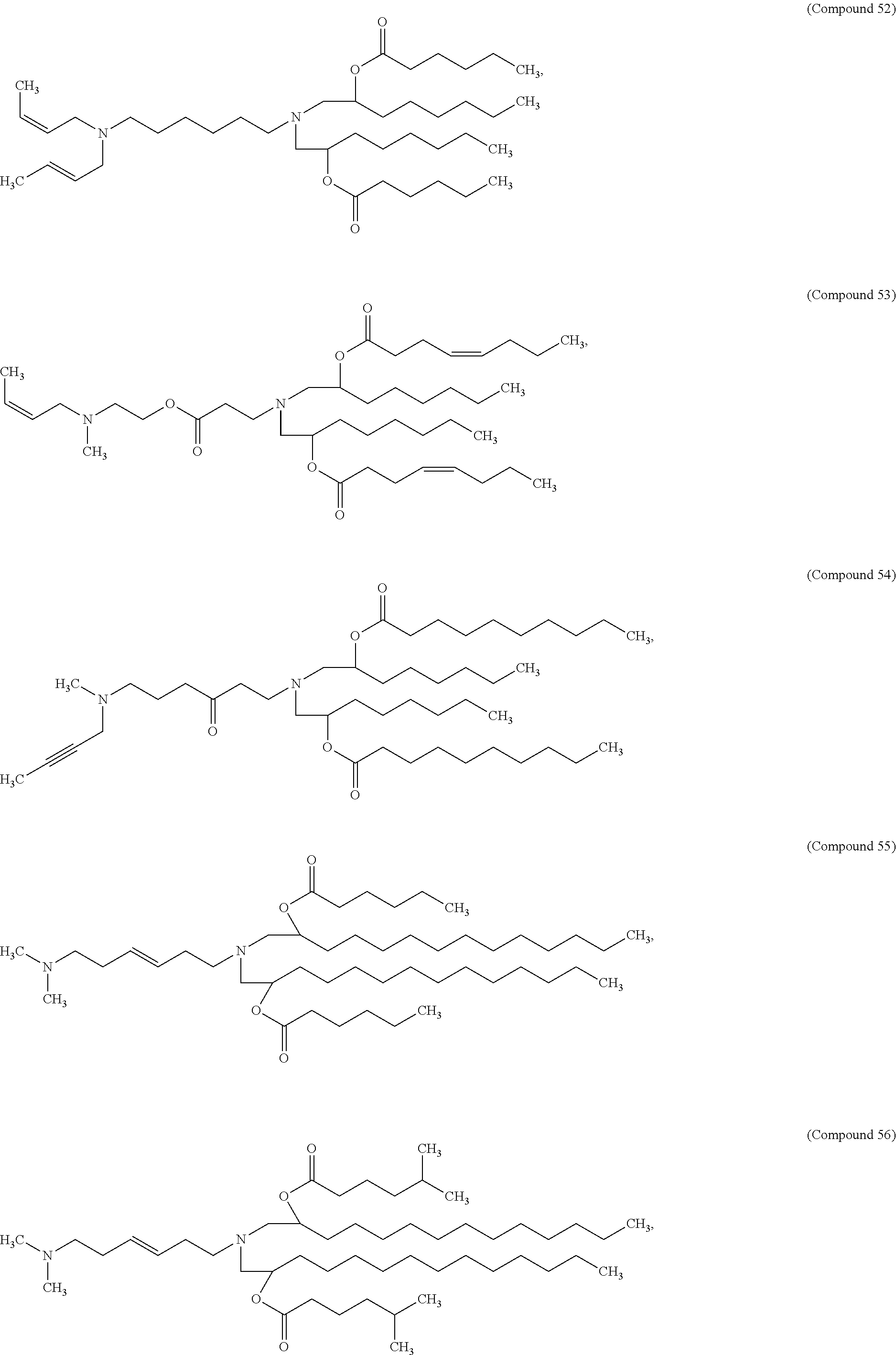 [SMILES: C/C=C\CN(C/C=C/C)CCCCCCN(CC(CCCCCC)OC(=O)CCCCC)CC(CCCCCC)OC(=O)CCCCC ; C/C=C\CN(C)CCOC(=O)CCN(CC(CCCCCC)OC(=O)CC/C=C\CCC)CC(CCCCCC)OC(=O)CC/C=C\CCC ; CC#CCN(C)CCCC(=O)CCN(CC(CCCCCC)OC(=O)CCCCCCCCC)CC(CCCCCC)OC(=O)CCCCCCCCC ; CCCCCCCCCCCCC(CN(CC/C=C/CCN(C)C)CC(CCCCCCCCCCCC)OC(=O)CCCCC)OC(=O)CCCCC ; CCCCCCCCCCCCC(CN(CC/C=C/CCN(C)C)CC(CCCCCCCCCCCC)OC(=O)CCCC(C)C)OC(=O)CCCC(C)C]

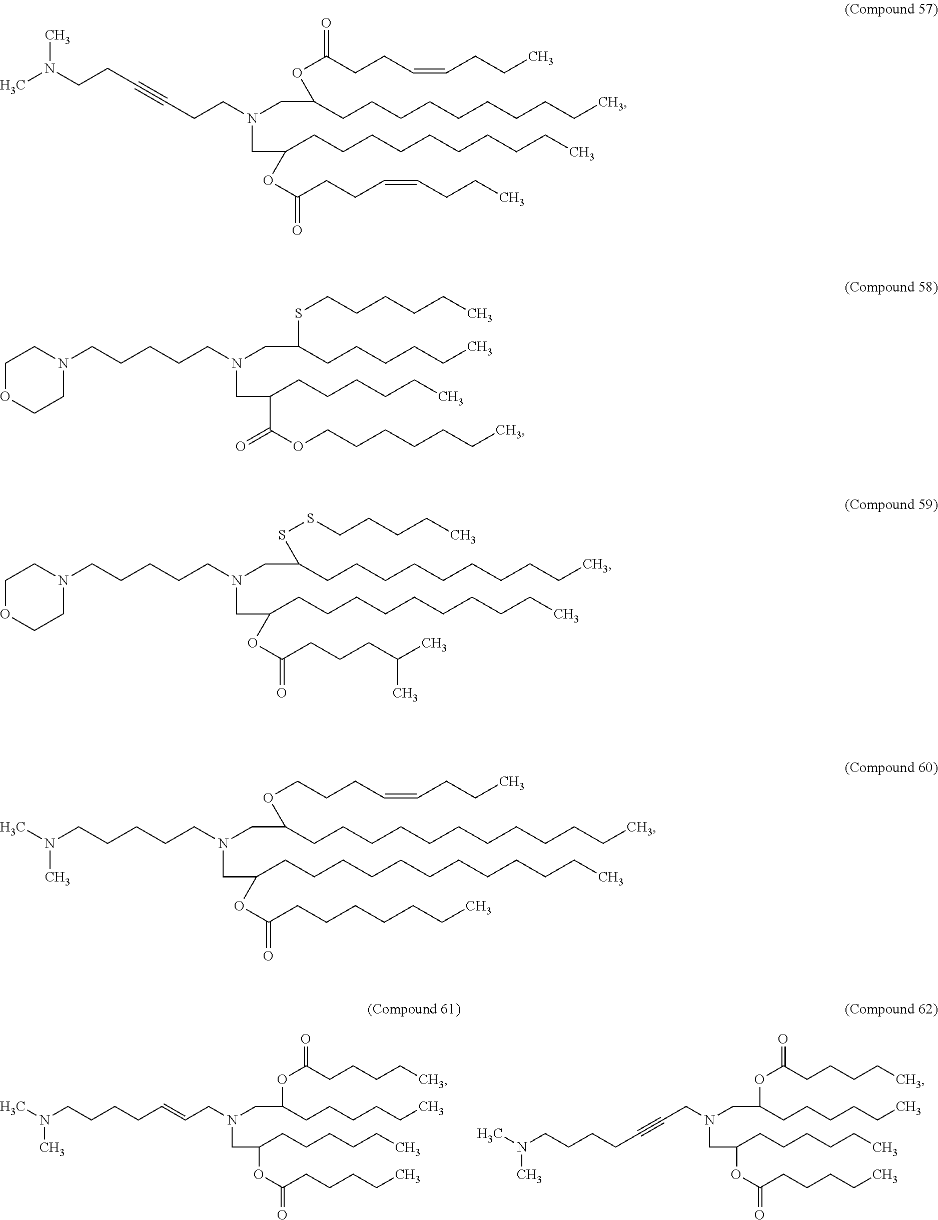 [SMILES: CCC/C=C\CCC(=O)OC(CCCCCCCCCC)CN(CCC#CCCN(C)C)CC(CCCCCCCCCC)OC(=O)CC/C=C\CCC ; CCCCCCCOC(=O)C(CCCCCC)CN(CCCCCN1CCOCC1)CC(CCCCCC)SCCCCCC ; CCCCCCCCCCC(CN(CCCCCN1CCOCC1)CC(CCCCCCCCCC)SSCCCCC)OC(=O)CCCC(C)C ; CCC/C=C\CCCOC(CCCCCCCCCCCC)CN(CCCCCN(C)C)CC(CCCCCCCCCCCC)OC(=O)CCCCCCC ; CCCCCCC(CN(C/C=C/CCCCN(C)C)CC(CCCCCC)OC(=O)CCCCC)OC(=O)CCCCC ; CCCCCCC(CN(CC#CCCCCN(C)C)CC(CCCCCC)OC(=O)CCCCC)OC(=O)CCCCC]

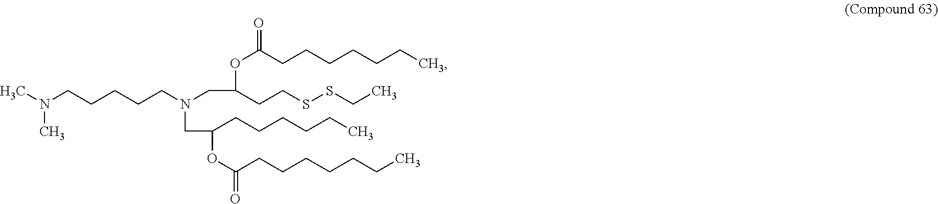 [SMILES: CCCCCCCC(=O)OC(CCCCCC)CN(CCCCCN(C)C)CC(CCSSCC)OC(=O)CCCCCCC]

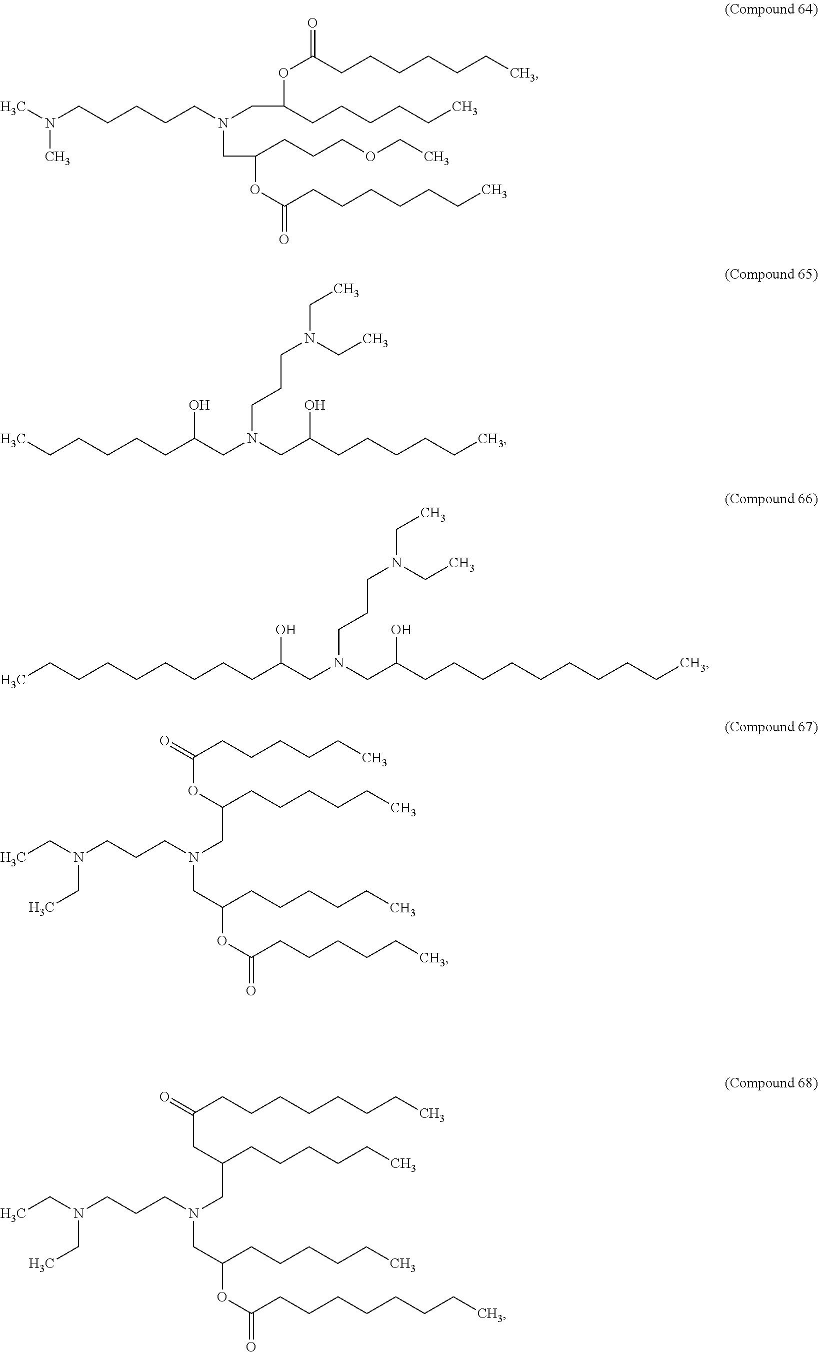 [SMILES: CCCCCCCC(=O)OC(CCCCCC)CN(CCCCCN(C)C)CC(CCCOCC)OC(=O)CCCCCCC ; CCCCCCC(O)CN(CCCN(CC)CC)CC(O)CCCCCC ; CCCCCCCCCCC(O)CN(CCCN(CC)CC)CC(O)CCCCCCCCC ; CCCCCCC(=O)OC(CCCCCC)CN(CCCN(CC)CC)CC(CCCCCC)OC(=O)CCCCCC ; CCCCCCCCC(=O)CC(CCCCCC)CN(CCCN(CC)CC)CC(CCCCCC)OC(=O)CCCCCCCC]

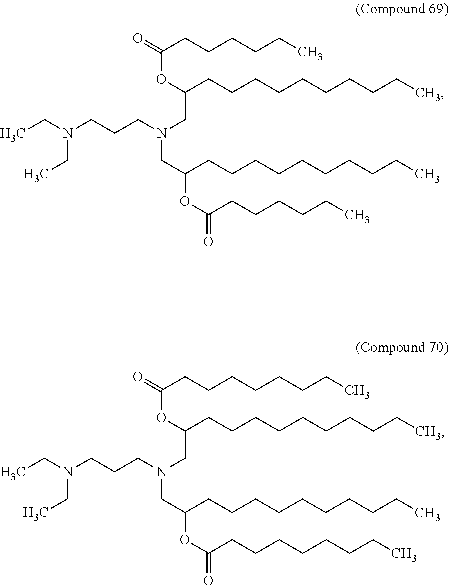 [SMILES: CCCCCCCCCCC(CN(CCCN(CC)CC)CC(CCCCCCCCCC)OC(=O)CCCCCC)OC(=O)CCCCCC ; CCCCCCCCCCC(CN(CCCN(CC)CC)CC(CCCCCCCCCC)OC(=O)CCCCCCCC)OC(=O)CCCCCCCC]

and salts and isomers thereof.

In another aspect of the disclosure, the compounds described herein are of Formula II or a salt or isomer thereof

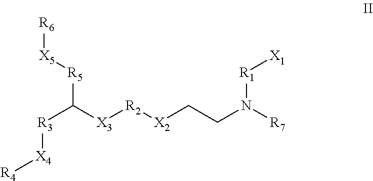

wherein:
R₁ is a C₁₋₁₈ alkylene, C₂₋₁₈ alkenylene, or C₂₋₁₈ alkynylene;
R₂ is a C₁₋₁₈ alkylene, C₂₋₁₈ alkenylene, or C₂₋₁₈ alkynylene, C₃₋₈ carbocyclylene, 3- to 8-membered heterocyclylene, or C₁₋₁₈ heteroalkylene;
R₃ and R₅ are the same or different, each independently a bond or a C₁₋₂₈ alkylene, C₂₋₂₈ alkenylene, or C₂₋₂₈ alkynylene;
R₄, R₆, and R₇ are the same or different, each independently hydrogen or a C₁₋₂₈ alkyl, C₂₋₂₈ alkenyl, or C₂₋₂₈ alkynyl, each optionally substituted by one, two, or three substituents independently selected from —OR, —SR, —S—S—R, —O—CO—R, —CO—OR, —CO—NR^{a}R^{b}, —NR^{a}—CO—R, —O—CO—NR^{a}R^{b}, —NR^{a}—CO—OR, —NR^{a}R^{b}, and —S—CO—R, wherein R at each occurrence is independently hydrogen or a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl; and R^{a} and R^{b} are independently hydrogen or lower alkyl;
X₁ is —OH, —SH, —N(R)₂, a C₅₋₈ carbocyclyl, or a heterocyclyl, hydrogen, or absent, wherein R at each occurrence is independently a lower alkyl or hydrogen;
X₂ is —O—CO—, —CO—O—, —NR—CO—, or —CO—NR—, wherein R is a lower alkyl or hydrogen; and
X₃, X₄, and X₅ are each independently —O—CO—, —CO—O—, —NR—CO—, —CO—NR—, a bond, or absent, wherein R is a lower alkyl or hydrogen,
wherein the longest chain of atoms in the compound is between 18 and 70 atoms.

In some embodiments, X₂, X₃, and R₂ form a part of a 3- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the heterocyclic ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₁, X₁, and R₇ form a part of a 5- to 20-membered heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the heterocyclic ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, X₅, R₅, R₃, and X₄ together with the carbon atom to which they are attached form a part of a 3- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes.

In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₁, R₂, R₃, R₄, R₅, R₆, and R₇ are optionally substituted with one or more substituents, such as, but not limited to, alkyl, alkenyl, or alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₅ is a bond; and R₆ is hydrogen; and X₅ is absent.

In some embodiments, X₁ is a hydrogen.

In some embodiments, X₂ is —O—CO— or —CO—O—.

In some embodiments, R₇ is a linear C₁₈ alkenyl.

In some embodiments, R₁ and X₁ together are a 1,2-dihydroxypropanemoiety, pyrrolidinoethylamine moiety, or (2-hydroxyethyl)(ethyl)amino)ethylamine moiety.

In some embodiments, R₇ is a 2-hexyldecyl hexanoate moiety; X₂ is —O—CO— or —CO—O—; and R₂ is methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl, or ((2-hexyldecyl)thio)ethyl.

In some embodiments, in the compounds of Formula II:
R₁ is a C₁₋₈ alkylene, C₂₋₈ alkenylene, or C₂₋₈ alkynylene;
R₂ is a C₁₋₈ alkylene, C₂₋₈ alkenylene, C₂₋₈ alkenylene, or C₁₋₈ heteroalkylene;
R₃ and R₅ are the same or different, each independently a bond or a C₁₋₁₄ alkylene, C₂₋₁₄ alkenylene, or C₂₋₁₄ alkenylene;
R₄, R₆, and R₇ are the same or different, each independently a hydrogen or a C₁₋₁₄ alkylene, C₂₋₁₄ alkenylene, or C₂₋₁₄ alkynylene;
X₁ is —OH, —N(R)₂, a C₅₋₈ carbocycyl or a hydrogen wherein R at each occurrence is independently a lower alkyl or hydrogen;
X₂ is —O—CO— or —CO—O—; and
X₃, X₄, and X₅ are each independently, a bond, —O—CO—, or —CO—O—,
wherein the longest chain of atoms in the compound is between 18 and 70 atoms.

In some embodiments, the compound of Formula II may be selected from the Compounds of List 2:

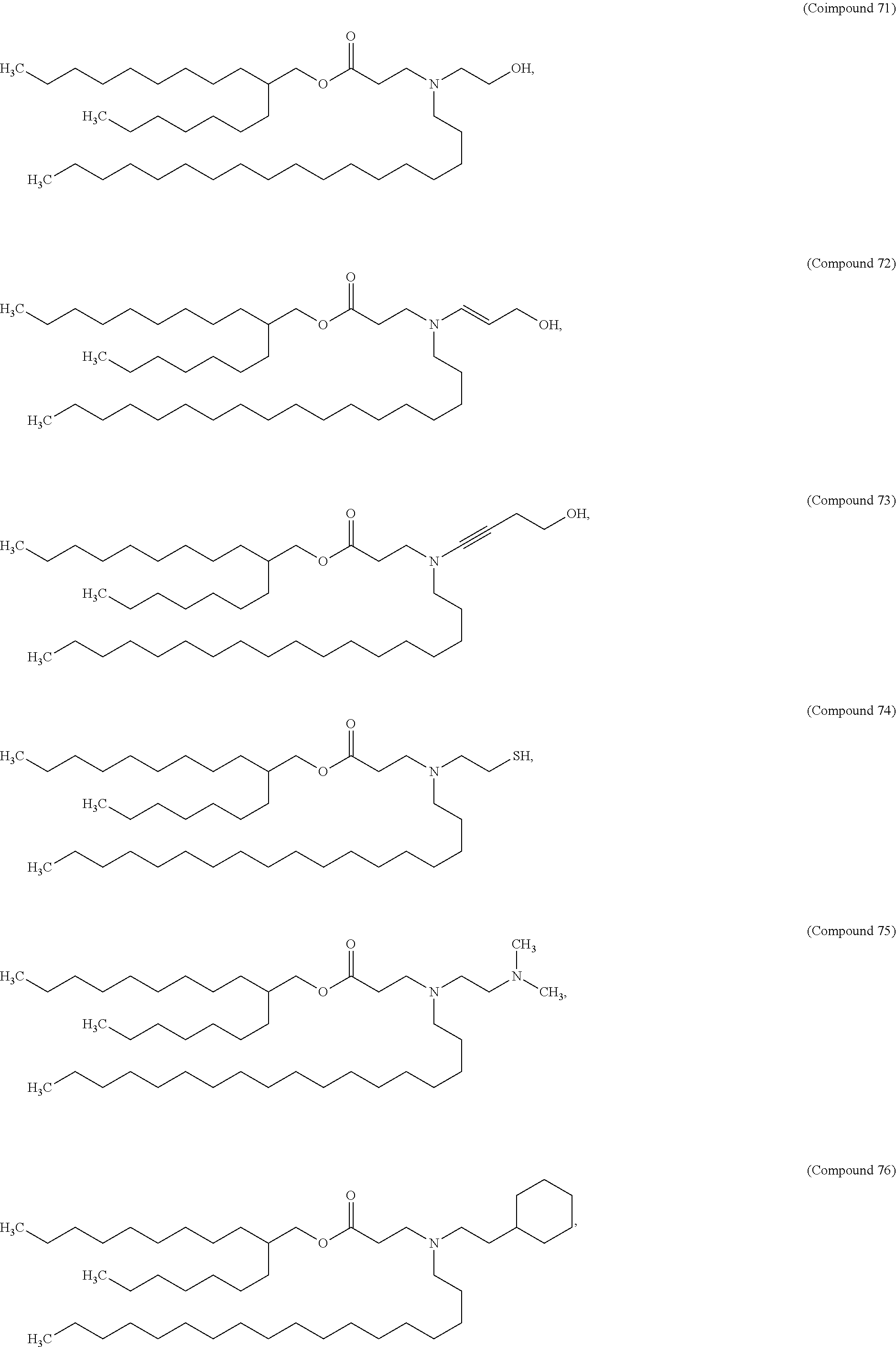 [SMILES: CCCCCCCCCCCCCCCCCCN(CCO)CCC(=O)OCC(CCCCCCC)CCCCCCCCC ; CCCCCCCCCCCCCCCCCCN(/C=C/CO)CCC(=O)OCC(CCCCCCC)CCCCCCCCC ; CCCCCCCCCCCCCCCCCCN(C#CCCO)CCC(=O)OCC(CCCCCCC)CCCCCCCCC ; CCCCCCCCCCCCCCCCCCN(CCS)CCC(=O)OCC(CCCCCCC)CCCCCCCCC ; CCCCCCCCCCCCCCCCCCN(CCC(=O)OCC(CCCCCCC)CCCCCCCCC)CCN(C)C ; CCCCCCCCCCCCCCCCCCN(CCC(=O)OCC(CCCCCCC)CCCCCCCCC)CCC1CCCCC1]

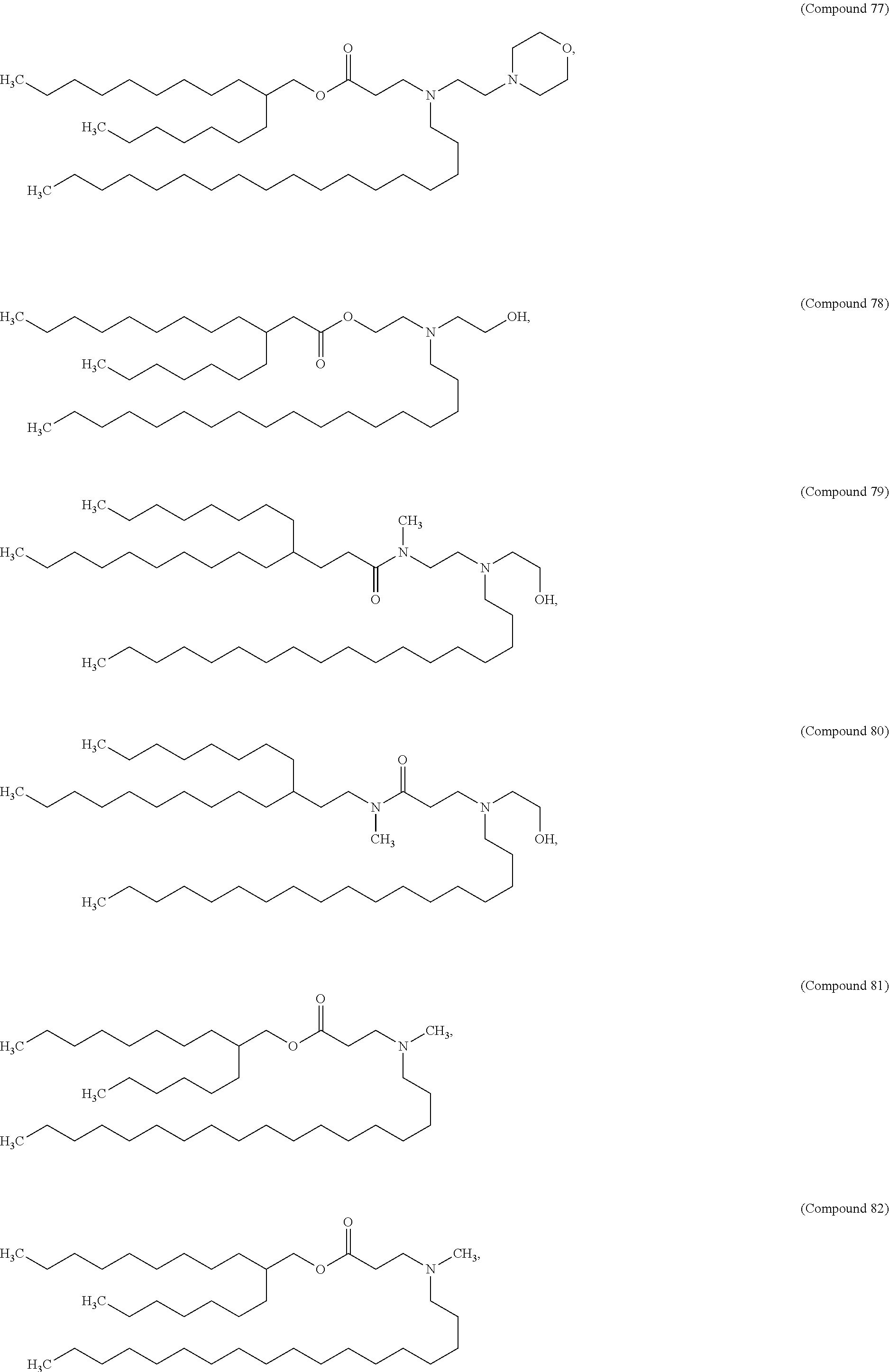 [SMILES: CCCCCCCCCCCCCCCCCCN(CCC(=O)OCC(CCCCCCC)CCCCCCCCC)CCN1CCOCC1 ; CCCCCCCCCCCCCCCCCCN(CCO)CCOC(=O)CC(CCCCCCC)CCCCCCCCC ; CCCCCCCCCCCCCCCCCCN(CCO)CCN(C)C(=O)CCC(CCCCCCCC)CCCCCCCCCC ; CCCCCCCCCCCCCCCCCCN(CCO)CCC(=O)N(C)CCC(CCCCCCCC)CCCCCCCCCC ; CCCCCCCCCCCCCCCCCCN(C)CCC(=O)OCC(CCCCCC)CCCCCCCC ; CCCCCCCCCCCCCCCCCCN(C)CCC(=O)OCC(CCCCCCC)CCCCCCCCC]

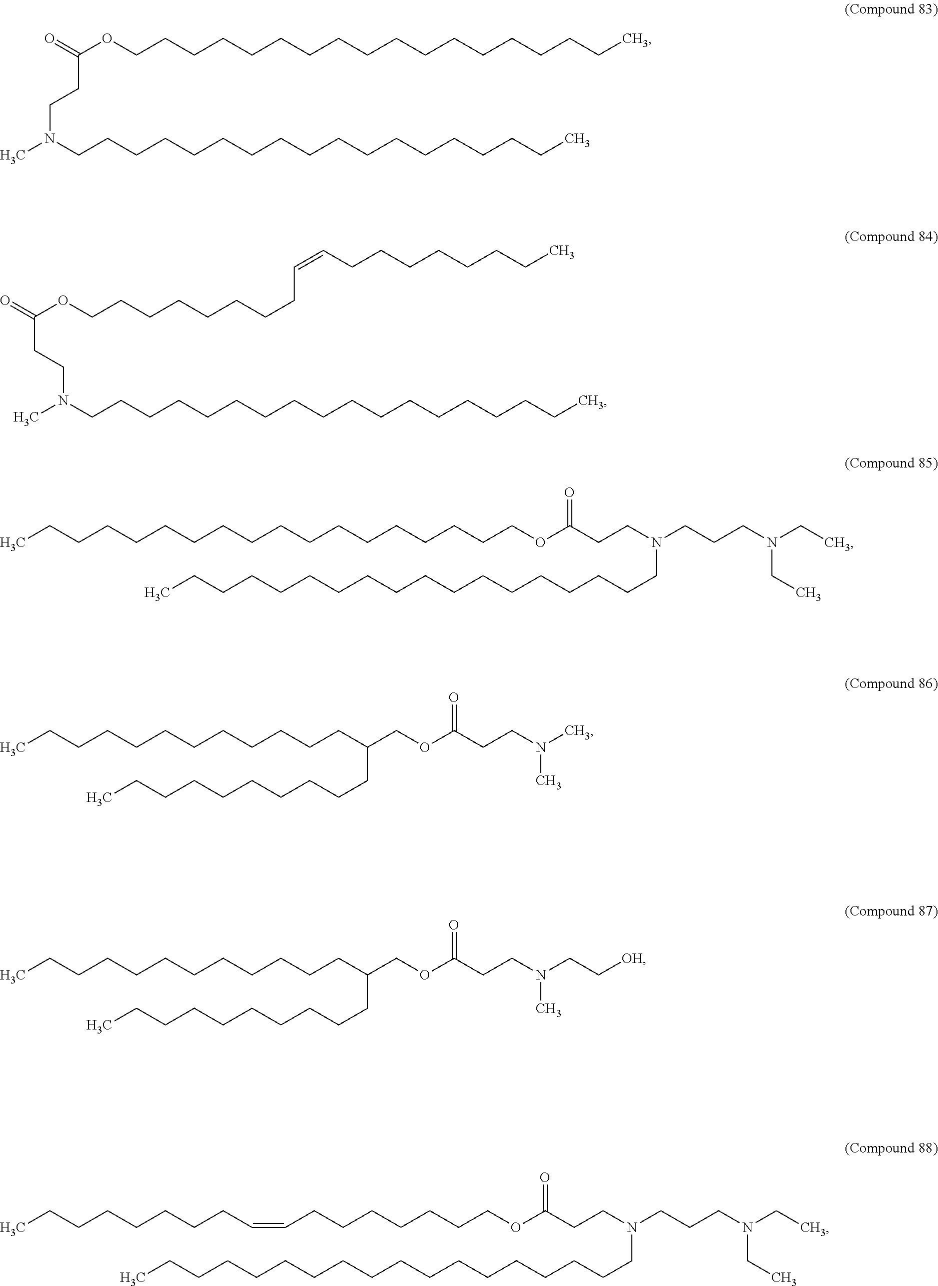 [SMILES: CCCCCCCCCCCCCCCCCCOC(=O)CCN(C)CCCCCCCCCCCCCCCCCC ; CCCCCCCC/C=C\CCCCCCCCOC(=O)CCN(C)CCCCCCCCCCCCCCCCCC ; CCCCCCCCCCCCCCCCCCOC(=O)CCN(CCCCCCCCCCCCCCCCCC)CCCN(CC)CC ; CCCCCCCCCCCCC(CCCCCCCCCC)COC(=O)CCN(C)C ; CCCCCCCCCCCCC(CCCCCCCCCC)COC(=O)CCN(C)CCO ; CCCCCCCC/C=C\CCCCCCCOC(=O)CCN(CCCCCCCCCCCCCCCCCC)CCCN(CC)CC]

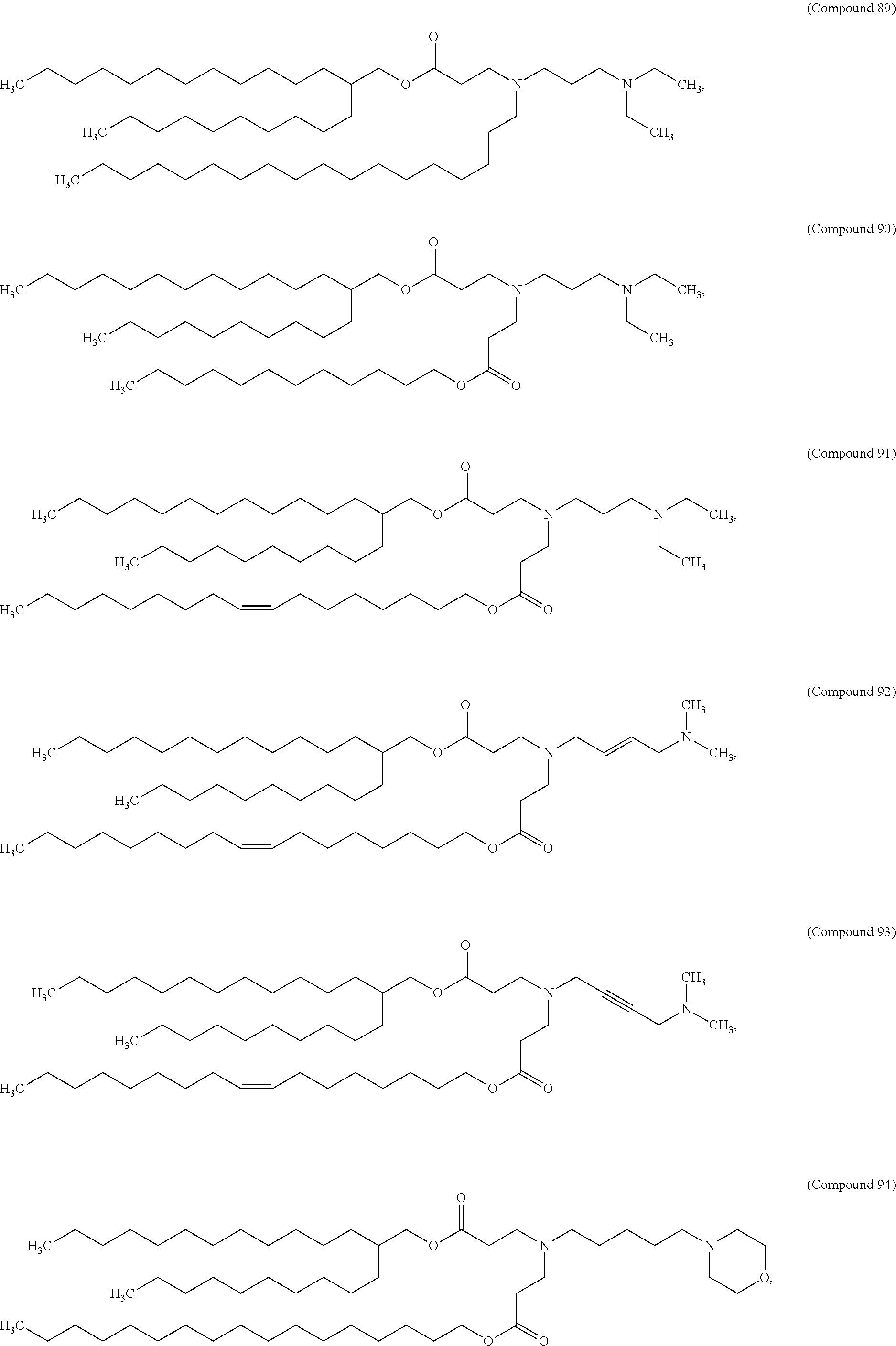 [SMILES: CCCCCCCCCCCCCCCCCCN(CCCN(CC)CC)CCC(=O)OCC(CCCCCCCCCC)CCCCCCCCCCCC ; CCCCCCCCCCCCOC(=O)CCN(CCCN(CC)CC)CCC(=O)OCC(CCCCCCCCCC)CCCCCCCCCCCC ; CCCCCCCC/C=C\CCCCCCCOC(=O)CCN(CCCN(CC)CC)CCC(=O)OCC(CCCCCCCCCC)CCCCCCCCCCCC ; CCCCCCCC/C=C\CCCCCCCOC(=O)CCN(C/C=C/CN(C)C)CCC(=O)OCC(CCCCCCCCCC)CCCCCCCCCCCC ; CCCCCCCC/C=C\CCCCCCCOC(=O)CCN(CC#CCN(C)C)CCC(=O)OCC(CCCCCCCCCC)CCCCCCCCCCCC ; CCCCCCCCCCCCCCCCCOC(=O)CCN(CCCCCN1CCOCC1)CCC(=O)OCC(CCCCCCCCCC)CCCCCCCCCCCC]

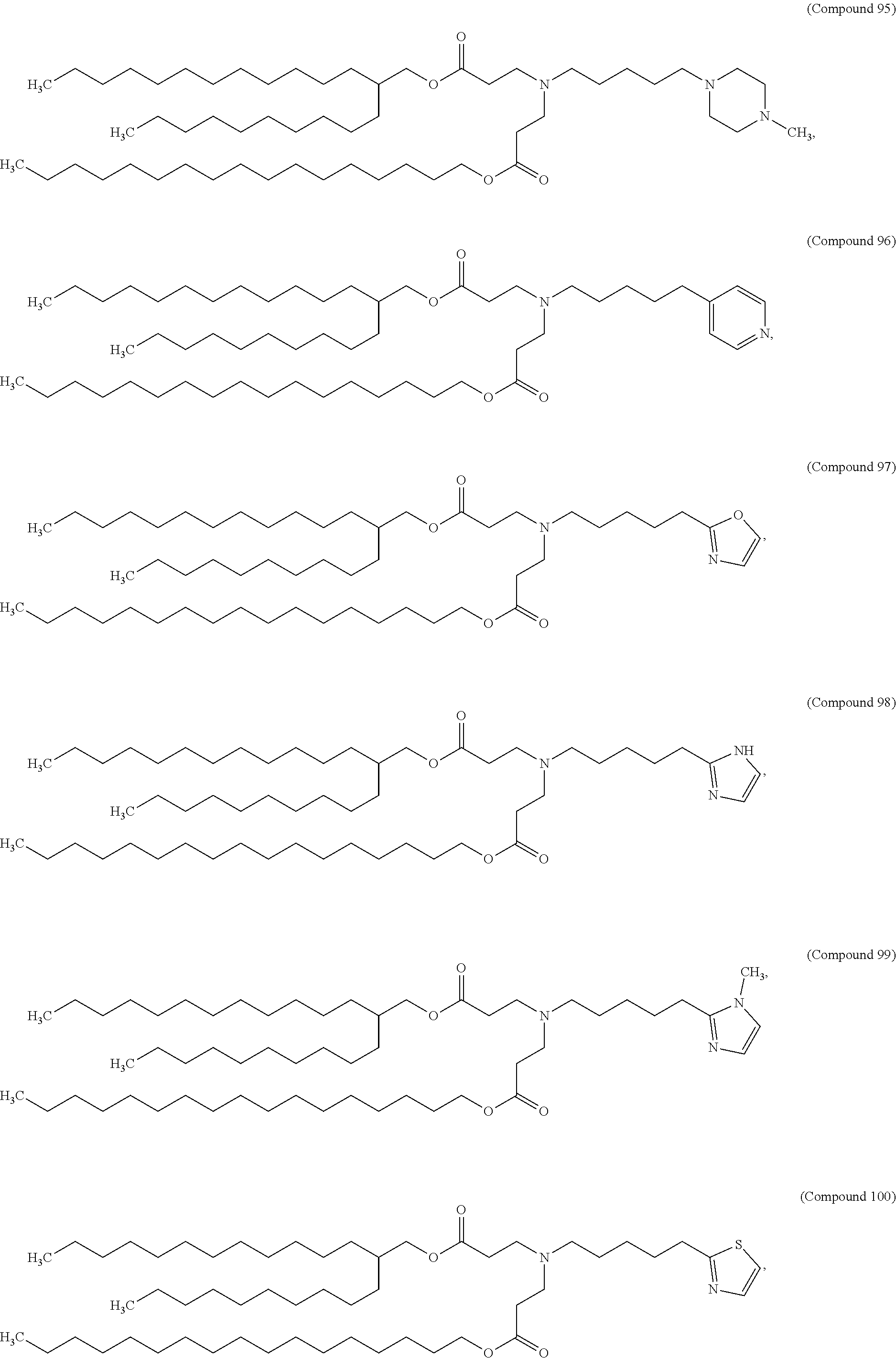 [SMILES: CCCCCCCCCCCCCCCCCOC(=O)CCN(CCCCCN1CCN(C)CC1)CCC(=O)OCC(CCCCCCCCCC)CCCCCCCCCCCC ; CCCCCCCCCCCCCCCCCOC(=O)CCN(CCCCCc1ccncc1)CCC(=O)OCC(CCCCCCCCCC)CCCCCCCCCCCC ; CCCCCCCCCCCCCCCCCOC(=O)CCN(CCCCCc1ncco1)CCC(=O)OCC(CCCCCCCCCC)CCCCCCCCCCCC ; CCCCCCCCCCCCCCCCCOC(=O)CCN(CCCCCc1ncc[nH]1)CCC(=O)OCC(CCCCCCCCCC)CCCCCCCCCCCC ; CCCCCCCCCCCCCCCCCOC(=O)CCN(CCCCCc1nccn1C)CCC(=O)OCC(CCCCCCCCCC)CCCCCCCCCCCC ; CCCCCCCCCCCCCCCCCOC(=O)CCN(CCCCCc1nccs1)CCC(=O)OCC(CCCCCCCCCC)CCCCCCCCCCCC]

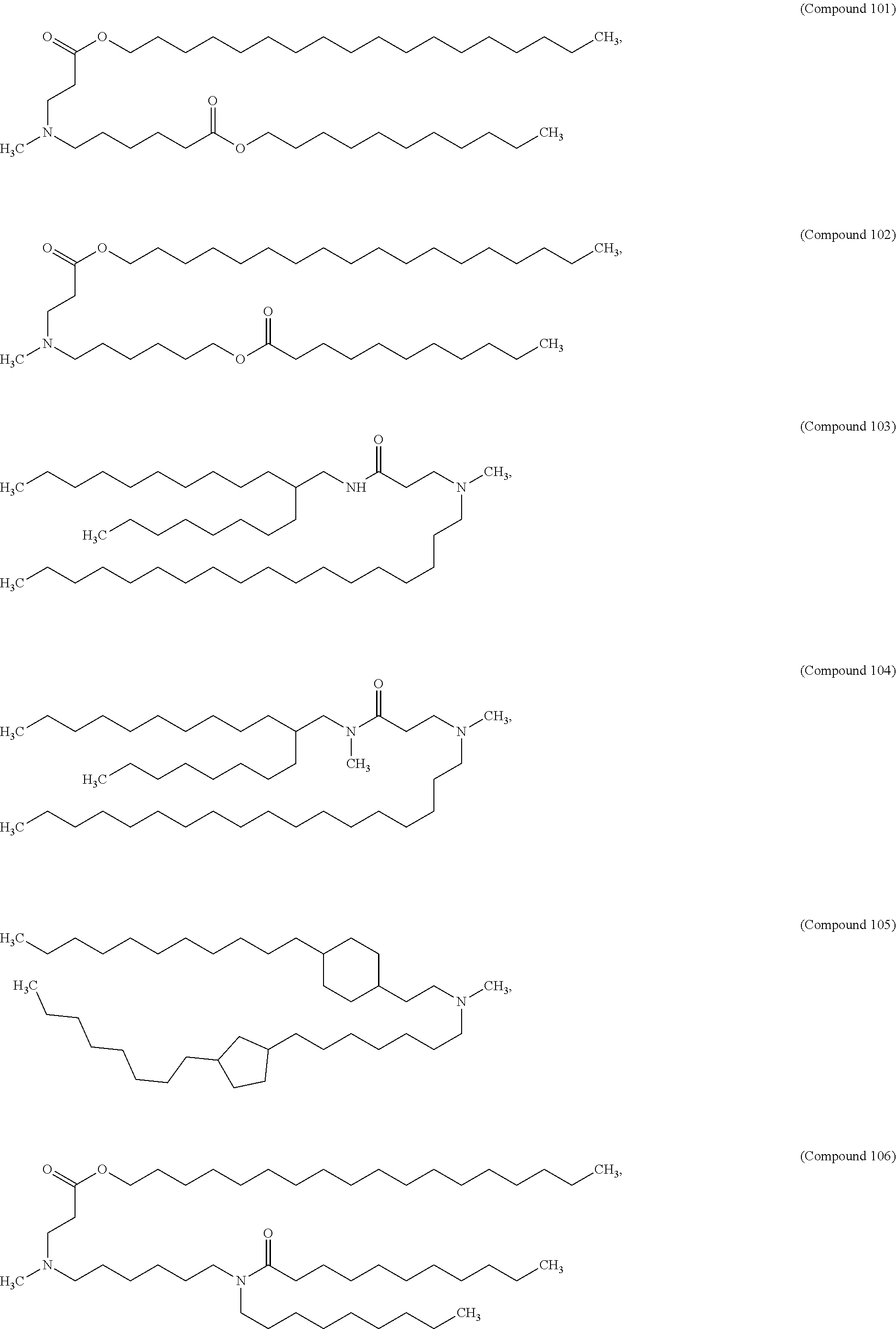 [SMILES: CCCCCCCCCCCCCCCCCCOC(=O)CCN(C)CCCCCC(=O)OCCCCCCCCCCC ; CCCCCCCCCCCCCCCCCCOC(=O)CCN(C)CCCCCCOC(=O)CCCCCCCCCC ; CCCCCCCCCCCCCCCCCCN(C)CCC(=O)NCC(CCCCCCCC)CCCCCCCCCC ; CCCCCCCCCCCCCCCCCCN(C)CCC(=O)N(C)CC(CCCCCCCC)CCCCCCCCCC ; CCCCCCCCCCCC1CCC(CCN(C)CCCCCCCC2CCC(CCCCCCCC)C2)CC1 ; CCCCCCCCCCCCCCCCCCOC(=O)CCN(C)CCCCCCN(CCCCCCCCC)C(=O)CCCCCCCCCC]

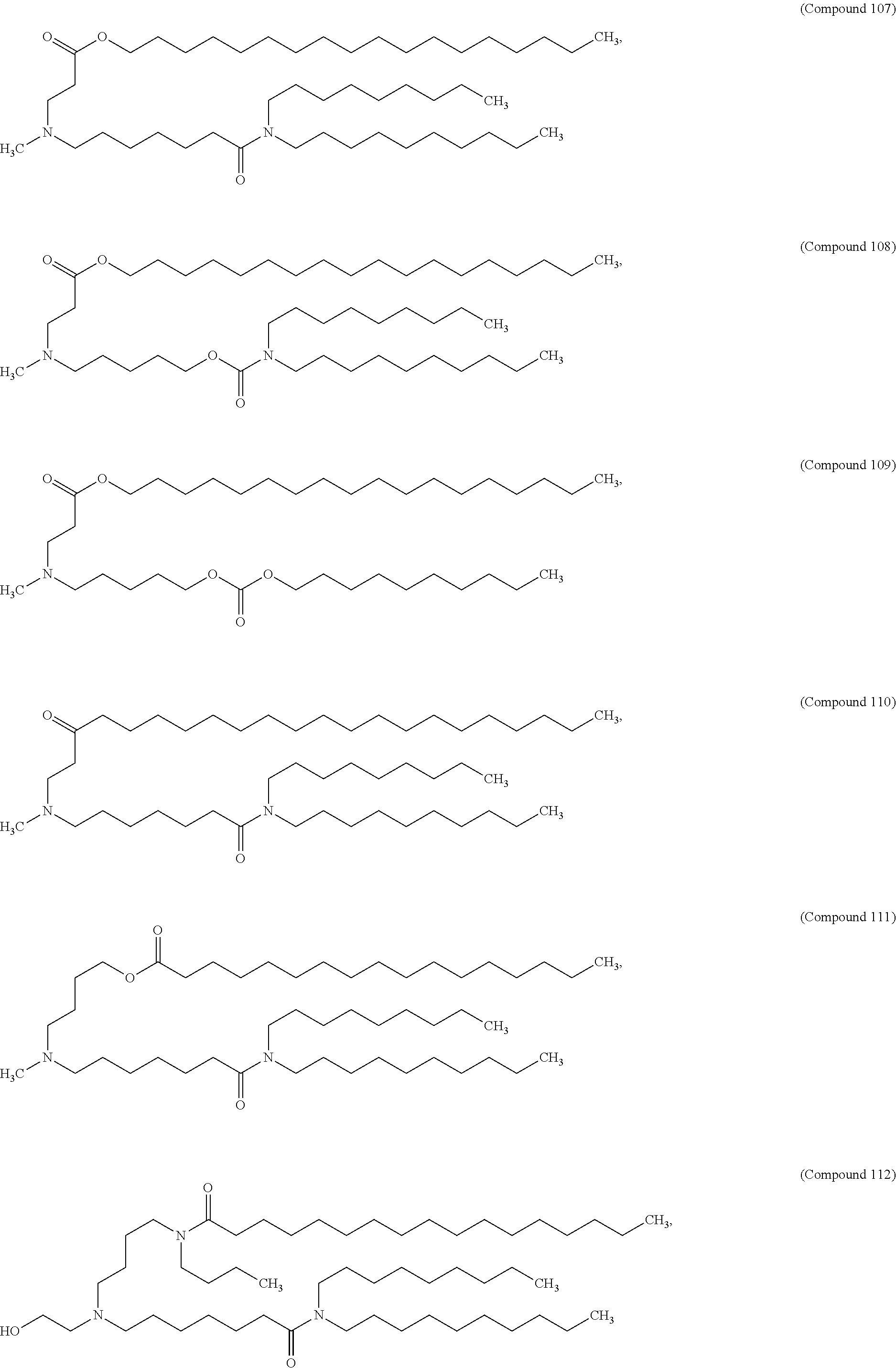 [SMILES: CCCCCCCCCCCCCCCCCCOC(=O)CCN(C)CCCCCCC(=O)N(CCCCCCCCC)CCCCCCCCCC ; CCCCCCCCCCCCCCCCCCOC(=O)CCN(C)CCCCCOC(=O)N(CCCCCCCCC)CCCCCCCCCC ; CCCCCCCCCCCCCCCCCCOC(=O)CCN(C)CCCCCOC(=O)OCCCCCCCCCC ; CCCCCCCCCCCCCCCCCCCC(=O)CCN(C)CCCCCCC(=O)N(CCCCCCCCC)CCCCCCCCCC ; CCCCCCCCCCCCCCCCC(=O)OCCCCN(C)CCCCCCC(=O)N(CCCCCCCCC)CCCCCCCCCC ; CCCCCCCCCCCCCCCCC(=O)N(CCCC)CCCCN(CCO)CCCCCCC(=O)N(CCCCCCCCC)CCCCCCCCCC]

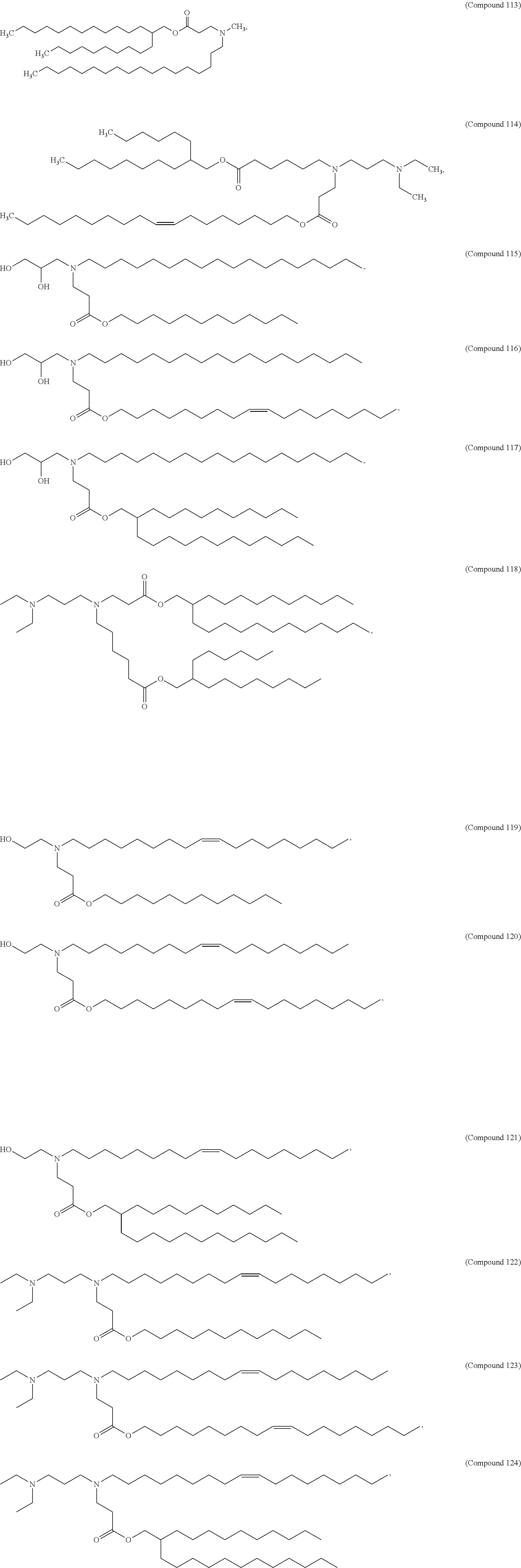

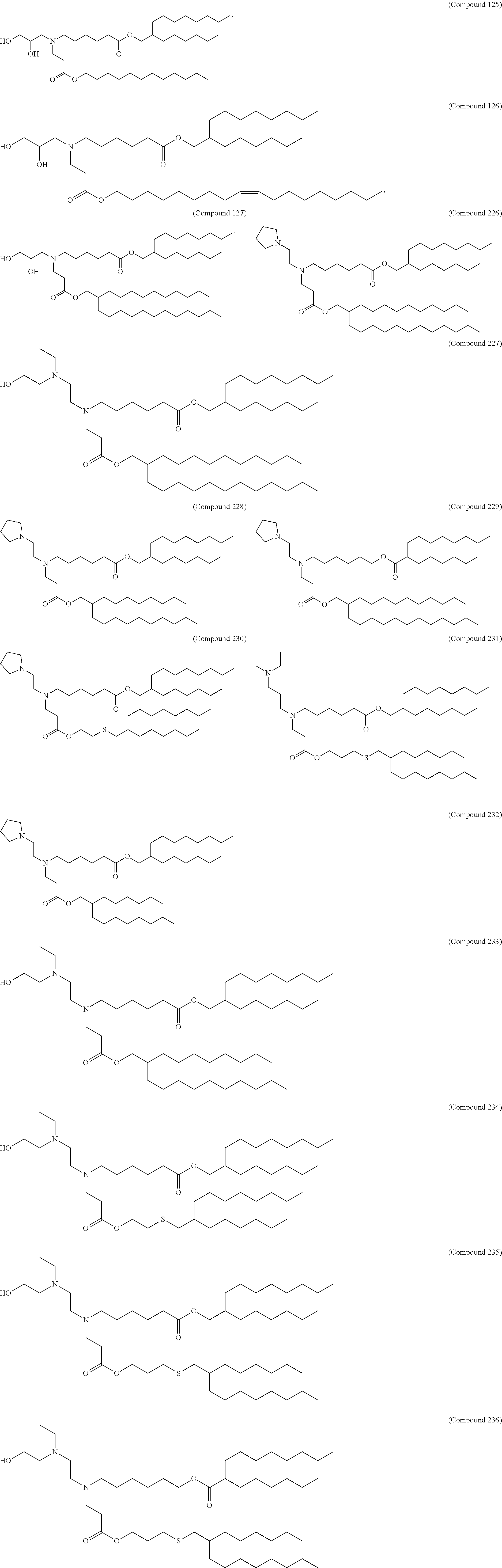

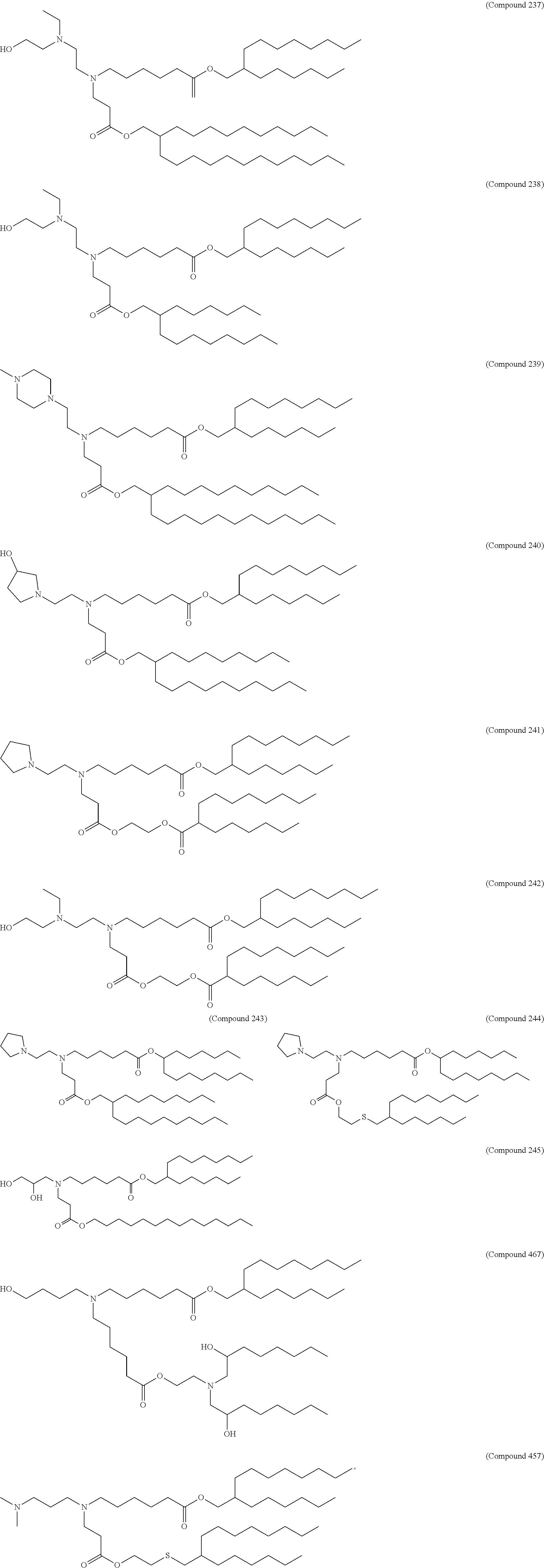

and salts, and isomers thereof.

In another aspect of the disclosure, the compounds described herein are of Formula III or a salt or isomer thereof

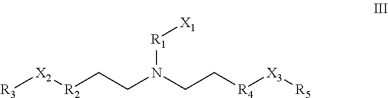

wherein:
R₁ is an optionally substituted C₁₋₁₈ alkylene, C₂₋₁₈ alkenylene, or C₂₋₁₈ alkynylene;
R₂ and R₄ are the same or different, each independently a bond or a C₁₋₂₈ alkylene, C₂₋₂₈ alkenylene, or C₂₋₂₈ alkynylene;
R₃ and R₅ are the same or different, each independently hydrogen or a C₁₋₂₈ alkyl, C₂₋₂₈ alkenyl, or C₂₋₂₈ alkynyl;
X₁ is —OH, —OR, —CO—OR, —CO—R, —O—CO—R, —SH, —SR, —N(R)₂, a C₅₋₈ carbocyclyl, a heterocyclyl, hydrogen, or absent, wherein R at each occurrence is independently hydrogen, or an optionally substituted methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl; and
X₂ and X₃ are the same or different, each independently —O—CO—, —CO—O—, —CO—, —NR—CO—, —CO—NR—, a bond, or absent, wherein R is a lower alkyl or hydrogen,
wherein the longest chain of atoms in the compound is between 18 to 70 atoms.

In some embodiments, R₁, and X₁ may together form a 5- to 8-membered aromatic or non-aromatic carbocycle or heterocycle having one or more heteroatoms selected from N, O, S, and P. In certain embodiments, the heterocycle formed by R₁ and X₁ may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₂ and R₄ may independently be a C₃₋₈ carbocycle or heterocycle having one or more heteroatoms selected from N, O, S, and P. The carbocycle or heterocycle groups may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₂ and X₂ and/or R₄ and X₃ may together with the atoms they are attached form a part of a 3- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups —OH, —SH, —OR, —SR, —NR₂, NHR, -oxo, —O—CO—, —NR—CO—, or other functional groups.

In some embodiments, R₄ and X₃ may together with the atoms they are attached be part of a 3- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups —OH, —SH, —OR, —SR, —NR₂, NHR, -oxo, —O—CO—, —NR—CO—, or other functional groups.

In some embodiments, X₂ and X₃ are independently —O—CO— or —CO—O—.

In some embodiments, R₃ and R₅ are linear C₁₀ alkylene.

In some embodiments, X₂ is —CO—O— and X₃ is —O—CO—.

In some embodiments, in the compounds of Formula III:
R₁ is an optionally substituted C₁₋₈ alkylene, C₂₋₈ alkenylene, or C₂₋₈ alkynylene;
R₂ and R₄ are the same or different, each independently, a bond or C₁₋₁₄ alkylene, C₂₋₁₄ alkenylene, or C₂₋₁₄ alkenylene;
R₃ and R₅ are the same or different, each independently hydrogen or C₁₋₈ alkyl, C₂₋₈ alkenyl, or C₂₋₈ alkenyl;
X₁ is —OH, —CO—OR, —O—CO—R, a C₅₋₈ carbocycyl, a heterocyclyl, or hydrogen, wherein R at each occurrence is independently hydrogen, or an optionally substituted methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl; and
X₂ and X₃ are the same or different, each independently —O—CO—, —CO—O—, —NR—CO—, —CO—NR—, or a bond, wherein R is a lower alkyl or hydrogen,
wherein the longest chain of atoms in the compound is between 18 to 70 atoms.

In some embodiments, the compound of Formula III may be selected from the Compounds of List 3:

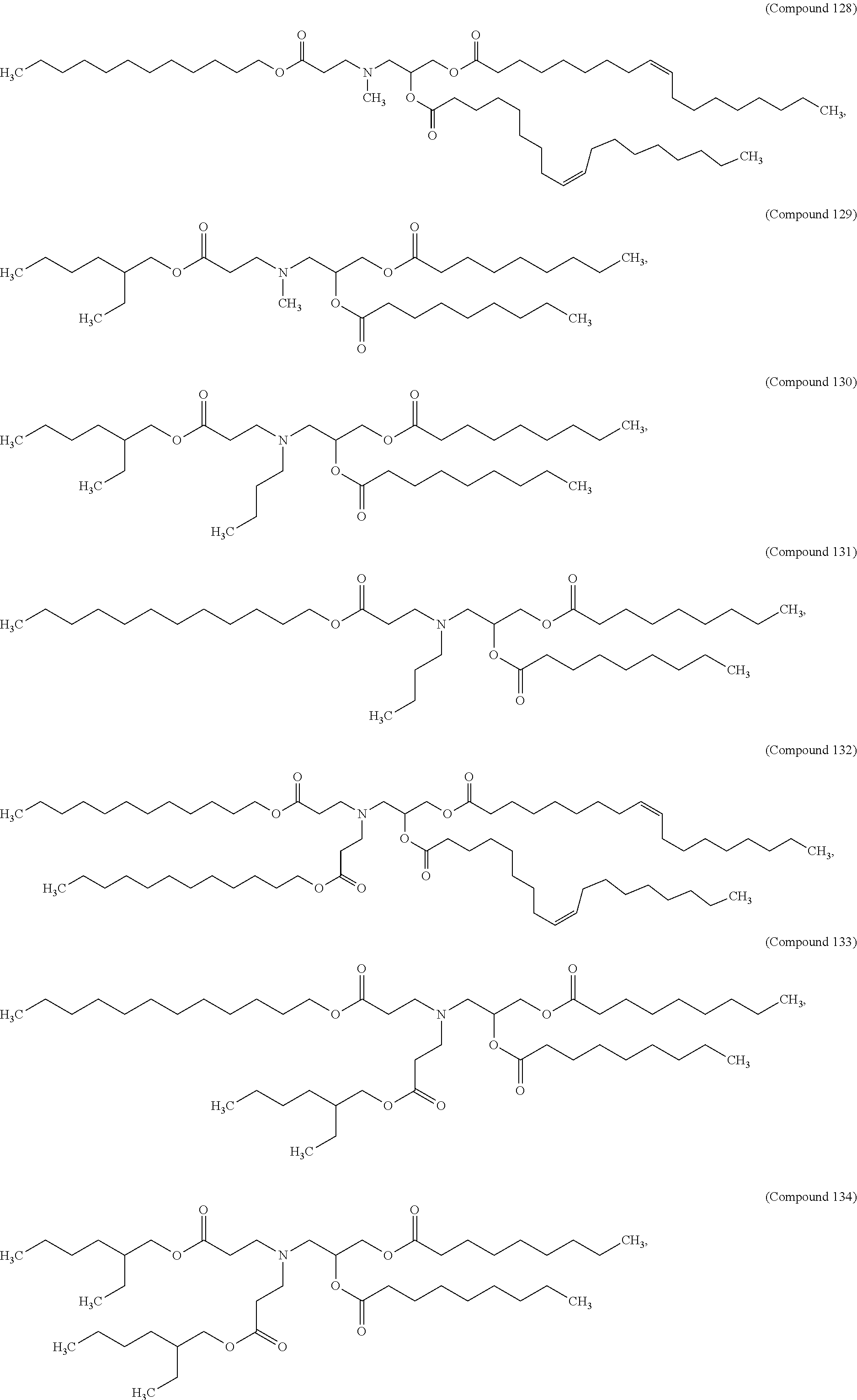

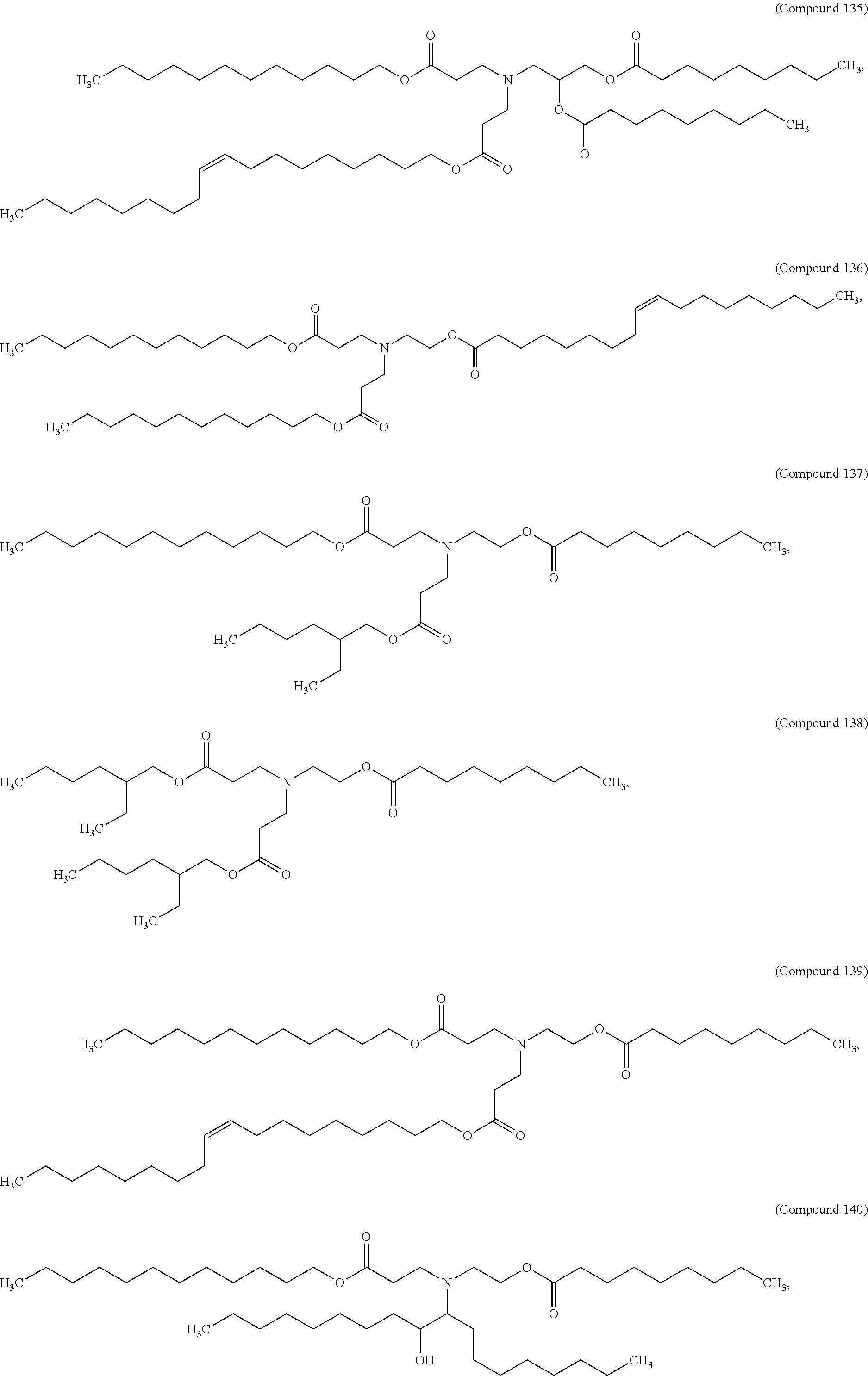 [SMILES: CCCCCCCC/C=C\CCCCCCCCOC(=O)CCN(CCC(=O)OCCCCCCCCCCCC)CC(COC(=O)CCCCCCCC)OC(=O)CCCCCCCC ; CCCCCCCC/C=C\CCCCCCCC(=O)OCCN(CCC(=O)OCCCCCCCCCCCC)CCC(=O)OCCCCCCCCCCCC ; CCCCCCCCCCCCOC(=O)CCN(CCOC(=O)CCCCCCCC)CCC(=O)OCC(CC)CCCC ; CCCCCCCCC(=O)OCCN(CCC(=O)OCC(CC)CCCC)CCC(=O)OCC(CC)CCCC ; CCCCCCCC/C=C\CCCCCCCCOC(=O)CCN(CCOC(=O)CCCCCCCC)CCC(=O)OCCCCCCCCCCCC ; CCCCCCCCCCCCOC(=O)CCN(CCOC(=O)CCCCCCCC)C(CCCCCCCC)C(O)CCCCCCCC]

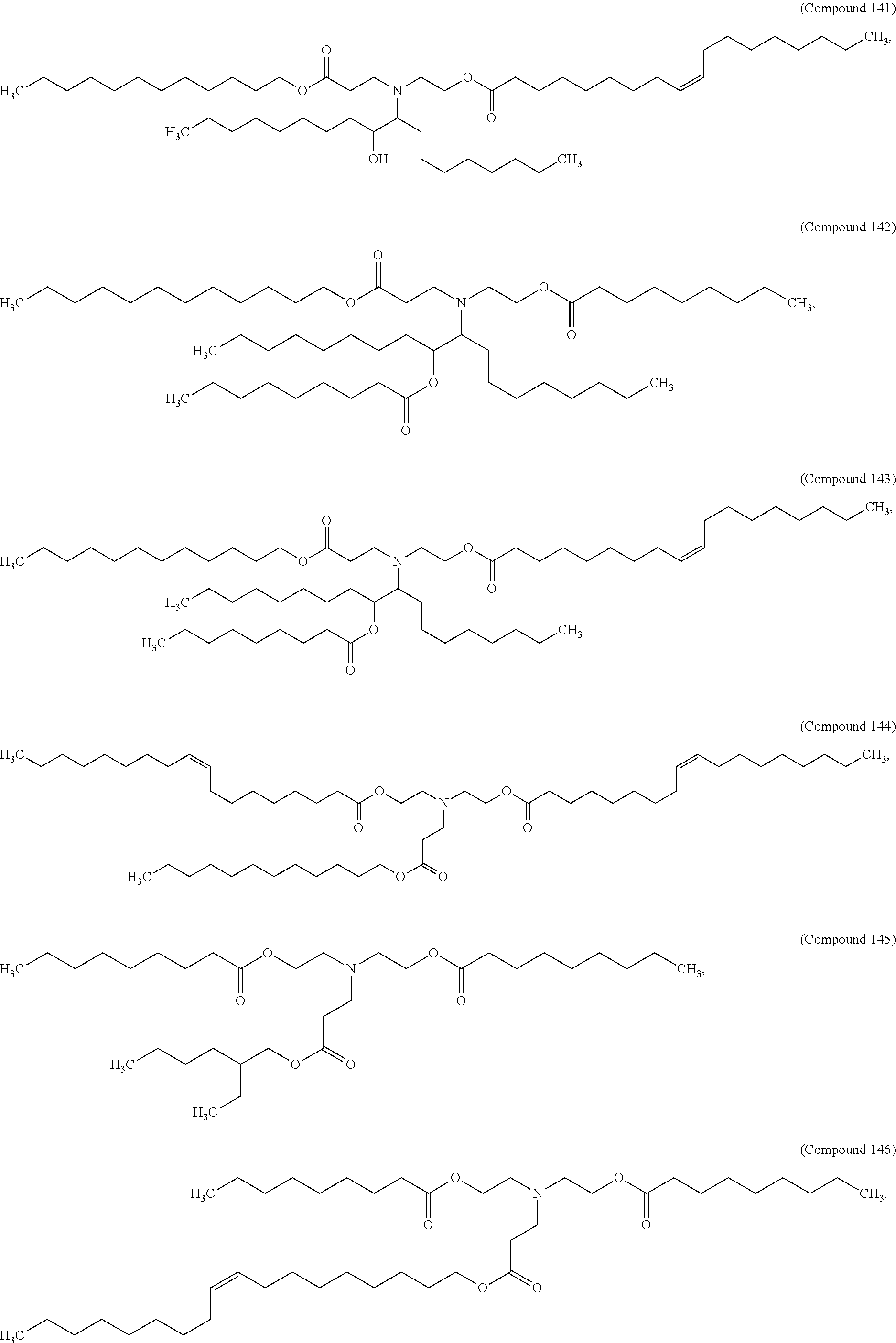 [SMILES: CCCCCCCC/C=C\CCCCCCCC(=O)OCCN(CCC(=O)OCCCCCCCCCCCC)C(CCCCCCCC)C(O)CCCCCCCC ; CCCCCCCCCCCCOC(=O)CCN(CCOC(=O)CCCCCCCC)C(CCCCCCCC)C(CCCCCCCC)OC(=O)CCCCCCCC ; CCCCCCCC/C=C\CCCCCCCC(=O)OCCN(CCC(=O)OCCCCCCCCCCCC)C(CCCCCCCC)C(CCCCCCCC)OC(=O)CCCCCCCC ; CCCCCCCC/C=C\CCCCCCCC(=O)OCCN(CCOC(=O)CCCCCCC/C=C\CCCCCCCC)CCC(=O)OCCCCCCCCCCCC ; CCCCCCCCC(=O)OCCN(CCOC(=O)CCCCCCCC)CCC(=O)OCC(CC)CCCC ; CCCCCCCC/C=C\CCCCCCCCOC(=O)CCN(CCOC(=O)CCCCCCCC)CCOC(=O)CCCCCCCC]

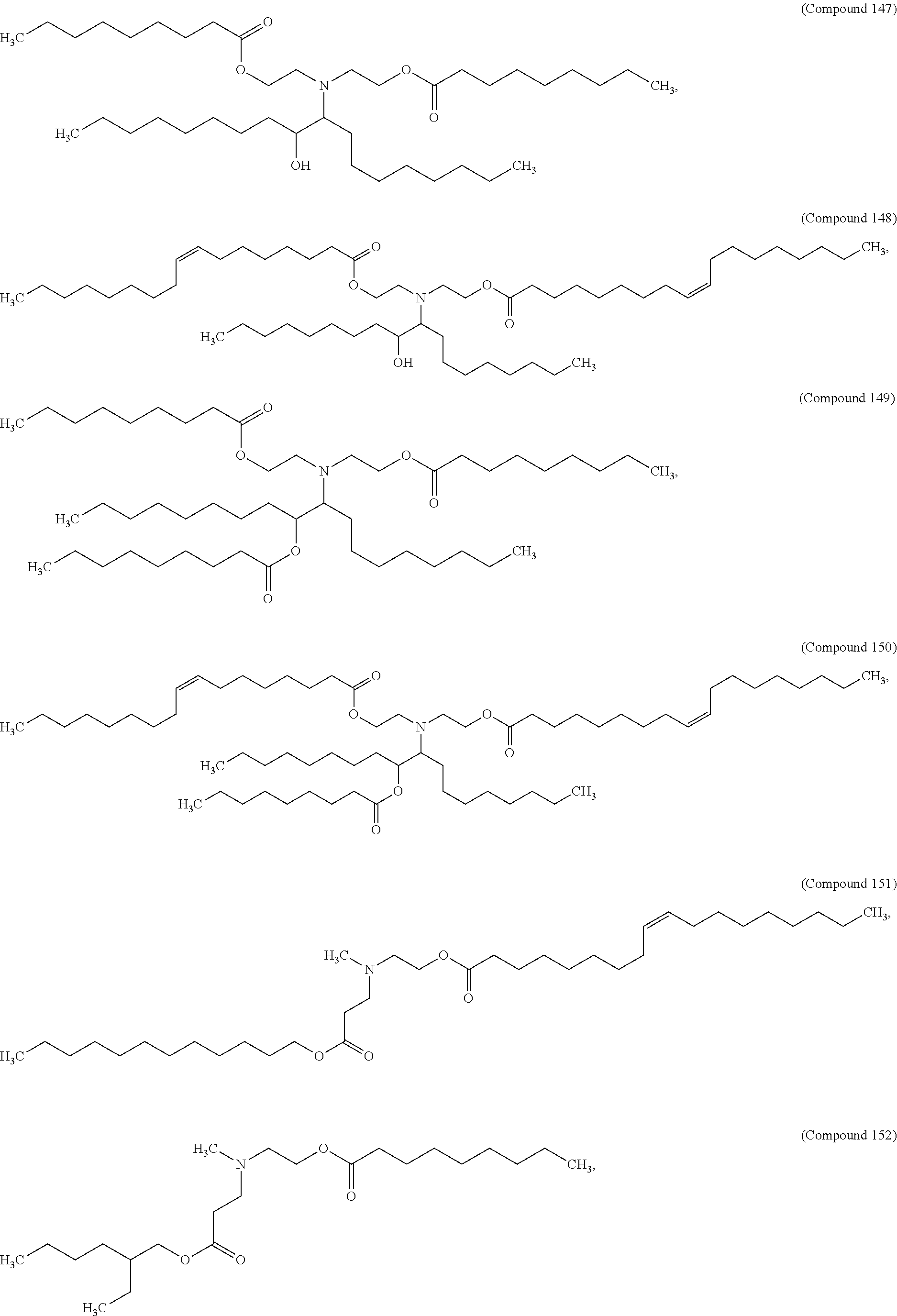 [SMILES: CCCCCCCCC(=O)OCCN(CCOC(=O)CCCCCCCC)C(CCCCCCCC)C(O)CCCCCCCC ; CCCCCCCC/C=C\CCCCCCCC(=O)OCCN(CCOC(=O)CCCCCC/C=C\CCCCCCCC)C(CCCCCCCC)C(O)CCCCCCCC ; CCCCCCCCC(=O)OCCN(CCOC(=O)CCCCCCCC)C(CCCCCCCC)C(CCCCCCCC)OC(=O)CCCCCCCC ; CCCCCCCC/C=C\CCCCCCCC(=O)OCCN(CCOC(=O)CCCCCC/C=C\CCCCCCCC)C(CCCCCCCC)C(CCCCCCCC)OC(=O)CCCCCCCC ; CCCCCCCC/C=C\CCCCCCCC(=O)OCCN(C)CCC(=O)OCCCCCCCCCCCC ; CCCCCCCCC(=O)OCCN(C)CCC(=O)OCC(CC)CCCC]

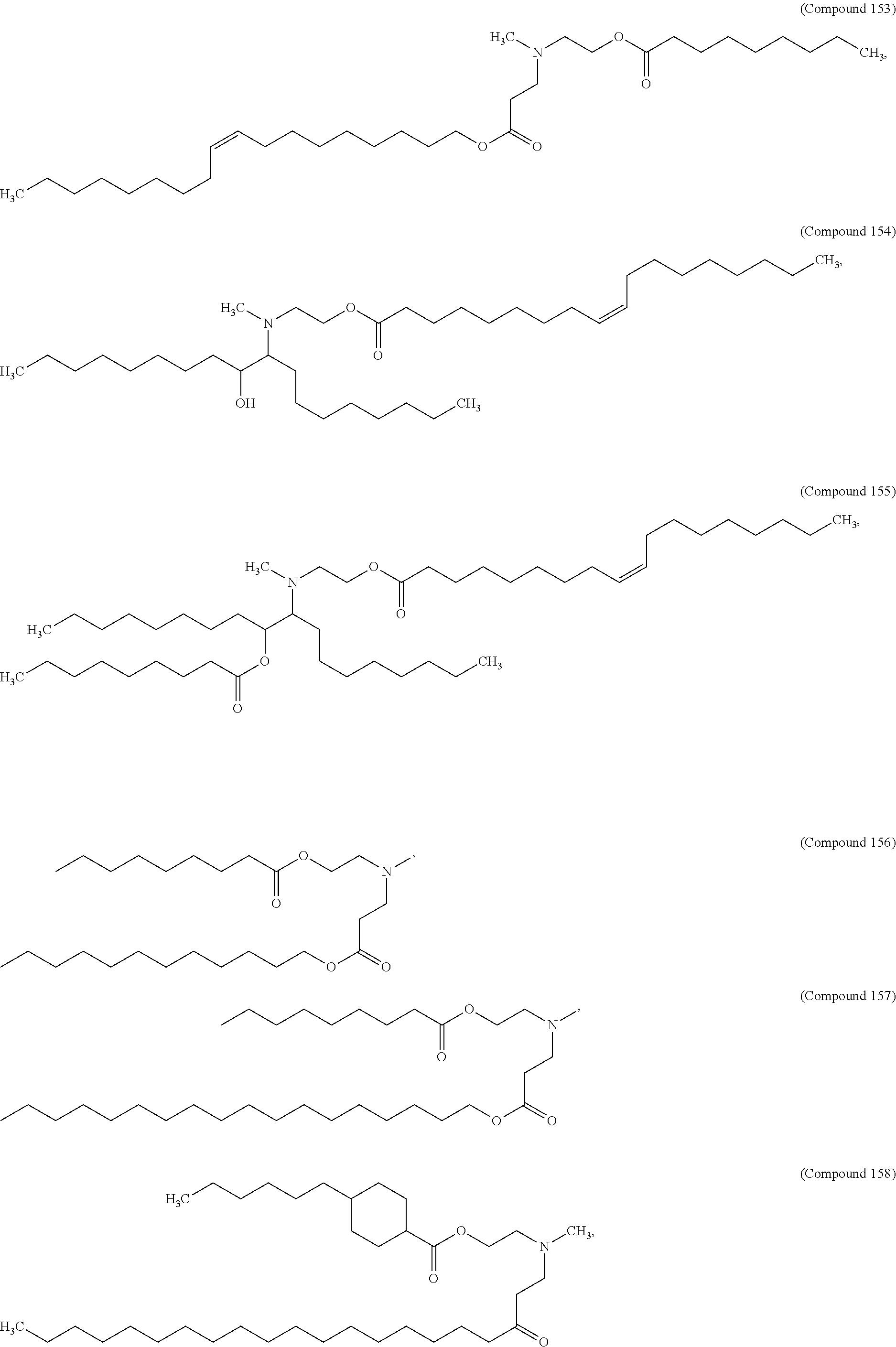

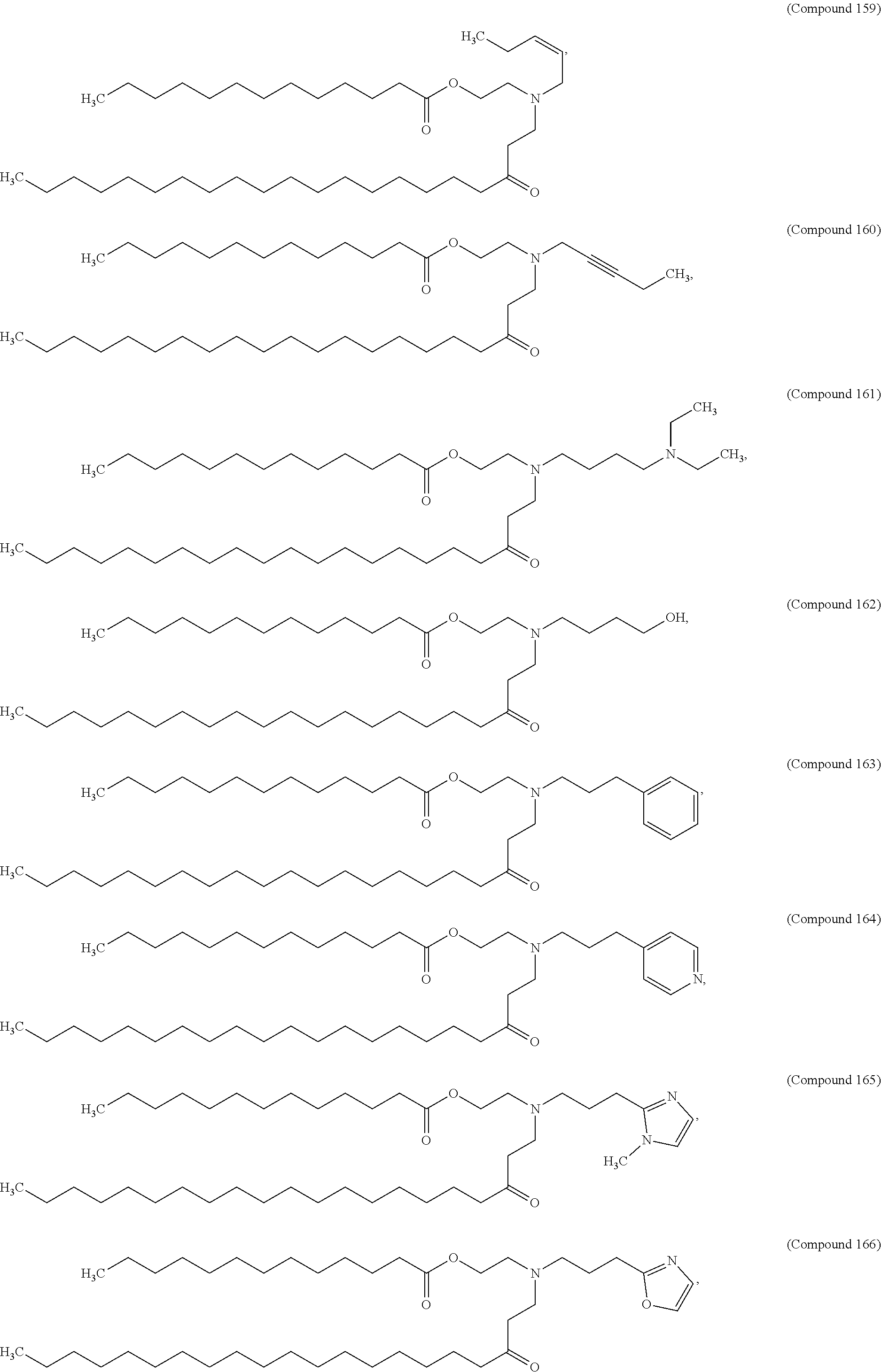

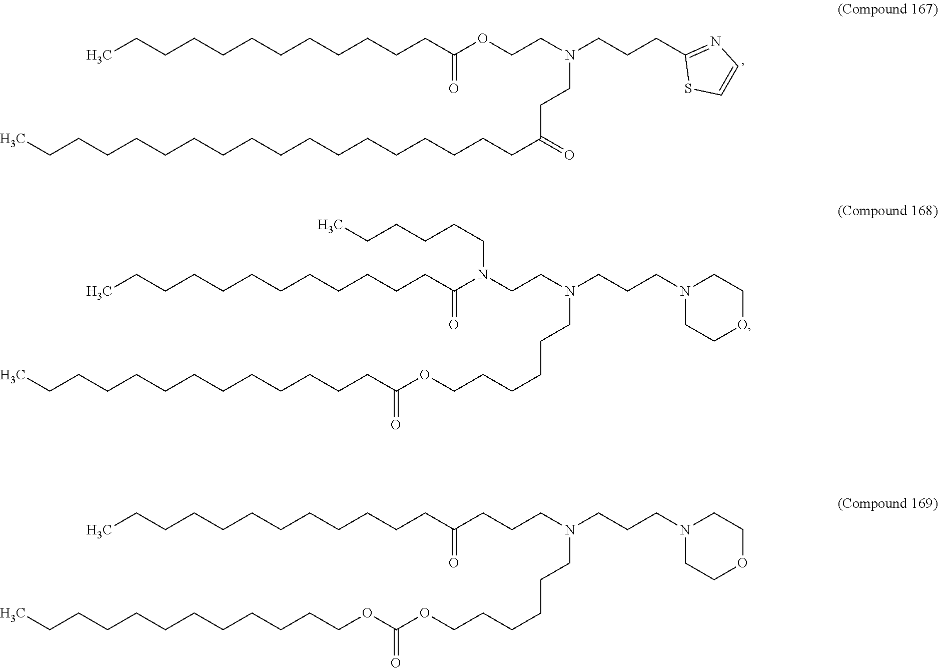 [SMILES: CCCCCCCCCCCCCCCCCCC(=O)CCN(CCCc1nccs1)CCOC(=O)CCCCCCCCCCCC ; CCCCCCCCCCCCCC(=O)OCCCCCCN(CCCN1CCOCC1)CCN(CCCCCC)C(=O)CCCCCCCCCCCC ; CCCCCCCCCCCCOC(=O)OCCCCCCN(CCCC(=O)CCCCCCCCCCCC)CCCN1CCOCC1]

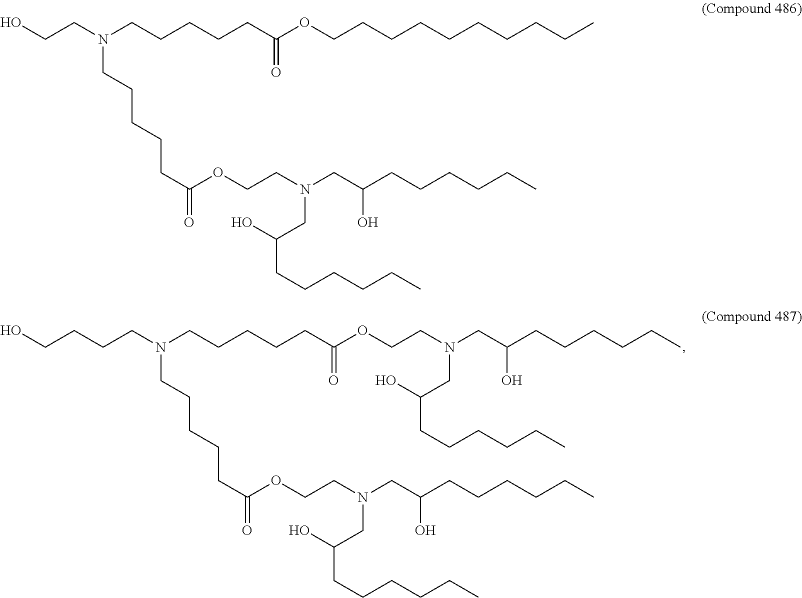

or salts or isomers thereof.

In another aspect of the disclosure, the compounds described herein are of Formula IV or a salt or isomer thereof

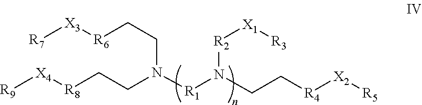

wherein:
R₁ is a C₁₋₁₈ alkylene, C₂₋₁₈ alkenylene, or C₂₋₁₈ alkynylene, each optionally substituted by one, two, or three substituents independently selected from —OR, —SR, —S—S—R, —O—CO—R, —CO—OR, —CO—NR^{a}R^{b}, —NR^{a}—CO—R, —O—CO—NR^{a}R^{b}, —NR^{a}—CO—OR, —NR^{a}R^{b}, and —S—CO—R, wherein R at each occurrence is independently hydrogen or lower alkyl; and R^{a} and R^{b} are independently hydrogen or lower alkyl;
n is 0, 1, 2, 3, 4, 5, 6, 7, 8, 9, or 10;
R₂ is a C₁₋₁₈ alkylene, C₂₋₁₈ alkenylene, or C₂₋₁₈ alkynylene;
R₃ is a C₁₋₁₈ alkyl, C₂₋₁₈ alkenyl, or C₂₋₁₈ alkynyl, or a C₅₋₈ carbocyclyl, heterocyclyl, hydrogen, or absent;
R₄, R₆, and R₈ are the same or different, each independently a bond or a C₁₋₂₈ alkylene, C₂₋₂₈ alkenylene, or C₂₋₂₈ alkynylene;
R₅, R₇, and R₉ are the same or different, each independently a bond or a C₂₋₂₈ alkyl, C₂₋₂₈ alkenyl, or C₂₋₂₈ alkynyl;
X₁ is a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent; and
X₂, X₃, and X₄ are the same or different, each independently a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent,
wherein the longest chain of atoms in the compound is between 18 and 70 atoms.

In some embodiments, R₁ may together with the one or both atoms to which it is attached, be part of a 3- to 10-membered heterocyclic ring having one or more of the heteroatoms selected from N, O, S, or P, which can be optionally part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups —OH, —SH, —OR, —SR, —NR₂, NHR, -oxo, —O—CO—, —NR—CO—, or other functional groups.

In some embodiments, R₂ and X₁ may together form a 5- to 8-membered aromatic or non-aromatic carbocycle or heterocycle having one or more heteroatoms selected from N, O, S, and P. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₄, R₆, and R₈ may independently be a C₃₋₈ carbocycle or heterocycle having one or more heteroatoms selected from N, O, S, and P. The carbocycle or heterocycle groups may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₄ and X₂ may together with the atoms they are attached form a part of a 3- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₆ and X₃ may together with the atoms they are attached form a part of a 3- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₈ and X₄ may together with the atoms they are attached form a part of a 3- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups —OH, —SH, —OR, —SR, —NR₂, NHR, -oxo, —O—CO—, —NR—CO—, or other functional groups.

In some embodiments, n is 3.

In some embodiments, R₁ is methyl, ethyl, propyl, butyl, or methyoxymethyl, ethoxyethyl, or methoxyethyl.

In some embodiments, R₂ is ethyl.

In some embodiments, X₁ is —O—CO— or —CO—O—.

In some embodiments, R₅, R₇, and R₉ are the same and are each linear C₁₀ alkyl.

In some embodiments, in the compounds of Formula IV:
R₁ is a C₁₋₈ alkylene, C₂₋₈ alkenylene, or C₂₋₈ alkynylene, each optionally substituted by one, two, or three substituents independently selected from —OR, —O—CO—R, —CO—OR, —CO—NR^{a}R^{b}, —NR^{a}—CO—R, —O—CO—NR^{a}R^{b}, —NR^{a}—CO—OR, and —NR^{a}R^{b}, wherein R at each occurrence is independently hydrogen or lower alkyl; and R^{a} and R^{b} are independently hydrogen or lower alkyl;
n is 0, 1, 2, 3, 4, or 5;
R₂ is a C₁₋₈ alkylene, C₂₋₈ alkenylene, or C₂₋₈ alkynylene;
R₃ is a C₁₋₈ alkyl, C₂₋₈ alkenyl, or C₂₋₈ alkynyl, or a C₅₋₈ carbocyclyl, heterocyclyl, or hydrogen;
R₄, R₆, and R₈ are the same or different, each independently a bond or a C₁₋₁₄ alkylene, C₂₋₁₄ alkenylene, or C₂₋₁₄ alkynylene;
R₅, R₇, and R₉ are the same or different, each independently a bond or a C₂₋₁₄ alkyl, C₂₋₁₄ alkenyl, or C₂₋₁₄ alkynyl;
X₁ is a methylene (—CH₂—), —O—, —NR— in which R is a lower alkyl, —O—CO—, —CO—O—, a C₅₋₈ carbocyclylene, a heterocyclylene, or a bond; and
X₂, X₃, and X₄ are the same or different, each independently a methylene (—CH₂—), —O—, —NR— in which R is a lower alkyl, —O—CO—, —CO—O—, —CO—, —O—CO—O—, a C₅₋₈ carbocyclylene, a heterocyclylene, or a bond,
wherein the longest chain of atoms in the compound is between 18 to 70 atoms.

In some embodiments, the compound of Formula IV may be selected from the Compounds of List 4:

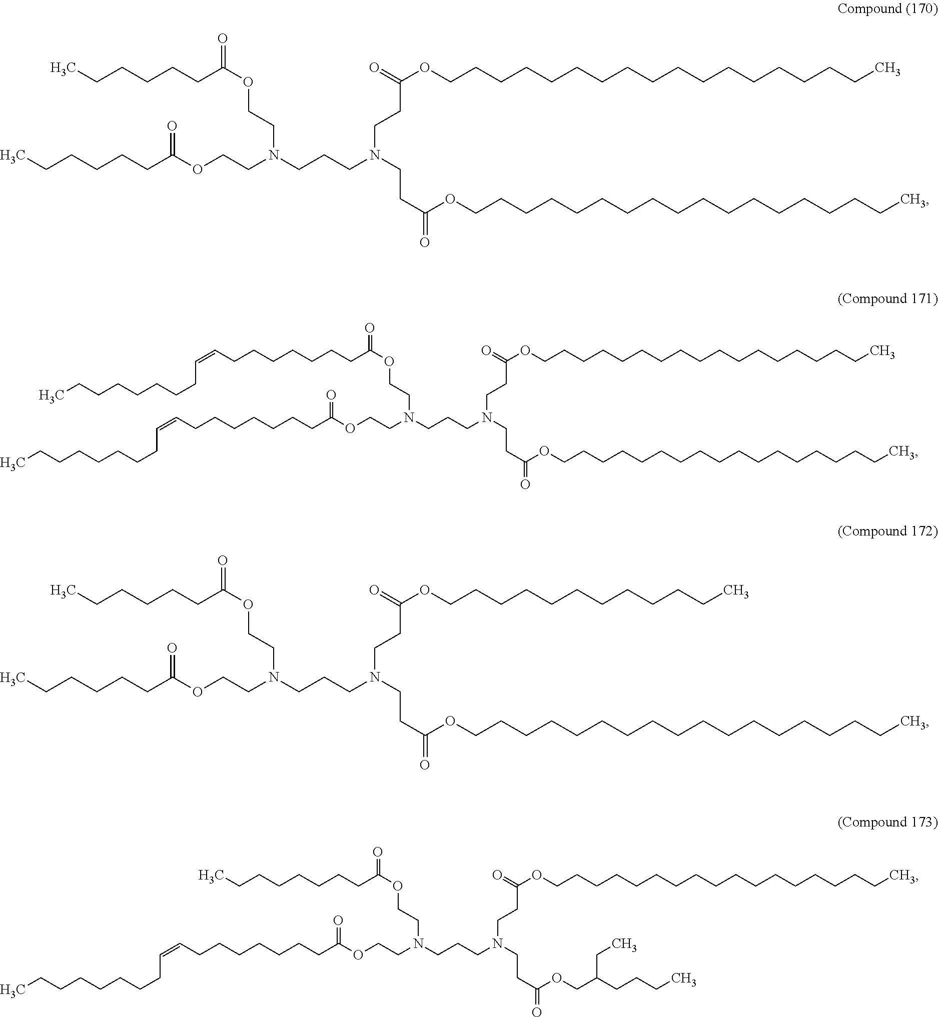 [SMILES: CCCCCCCCCCCCCCCCCCOC(=O)CCN(CCCN(CCOC(=O)CCCCCC)CCOC(=O)CCCCCC)CCC(=O)OCCCCCCCCCCCCCCCCCC ; CCCCCCCC/C=C\CCCCCCCC(=O)OCCN(CCCN(CCC(=O)OCCCCCCCCCCCCCCCCCC)CCC(=O)OCCCCCCCCCCCCCCCCCC)CCOC(=O)CCCCCCC/C=C\CCCCCCCC ; CCCCCCCCCCCCCCCCCCOC(=O)CCN(CCCN(CCOC(=O)CCCCCC)CCOC(=O)CCCCCC)CCC(=O)OCCCCCCCCCCCC ; CCCCCCCC/C=C\CCCCCCCC(=O)OCCN(CCCN(CCC(=O)OCCCCCCCCCCCCCCCCCC)CCC(=O)OCC(CC)CCCC)CCOC(=O)CCCCCCCC]

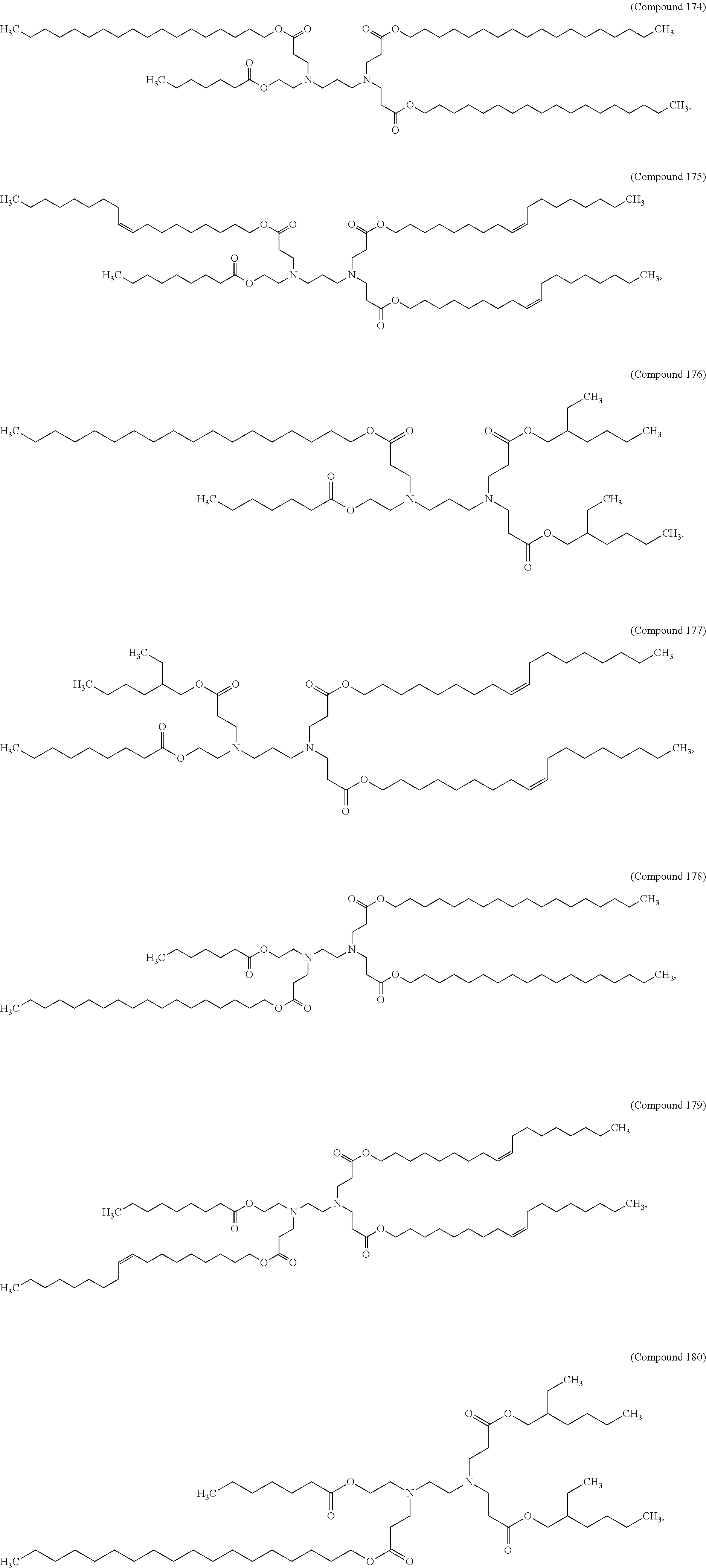

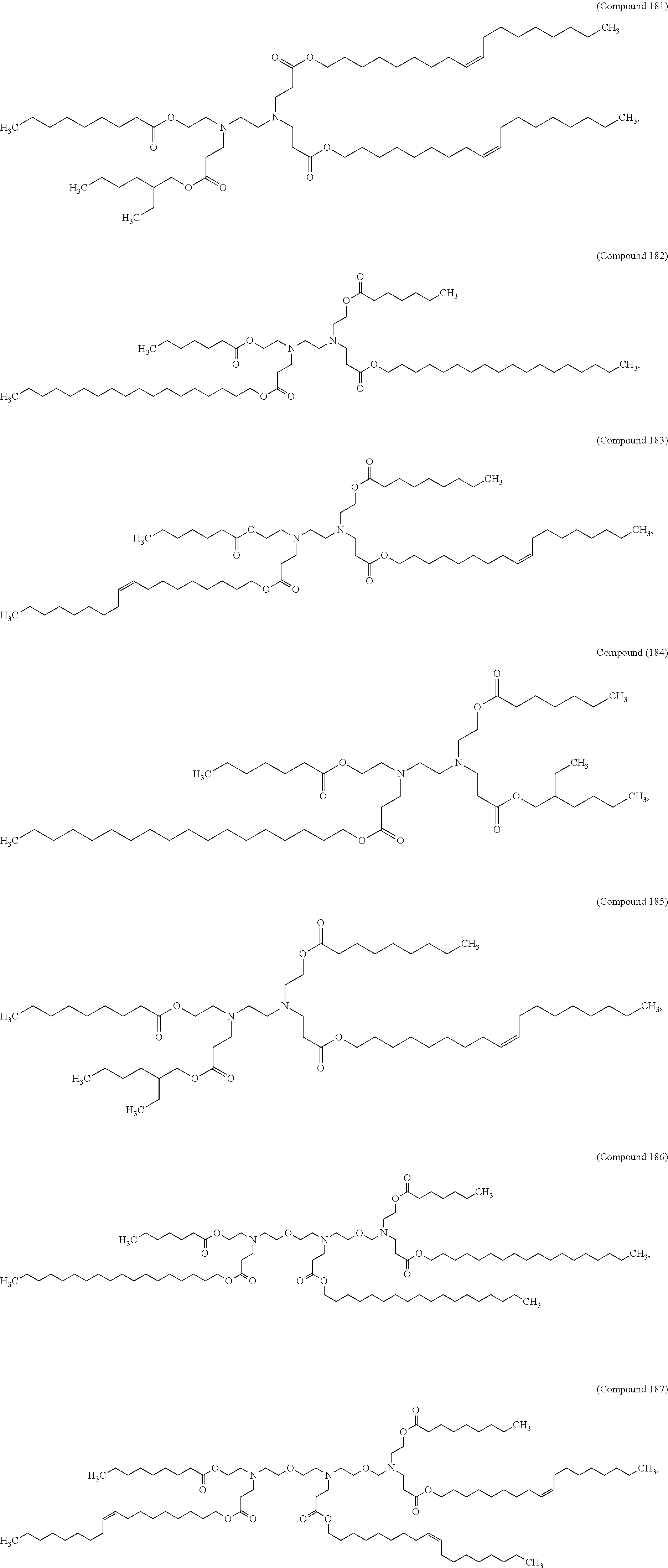

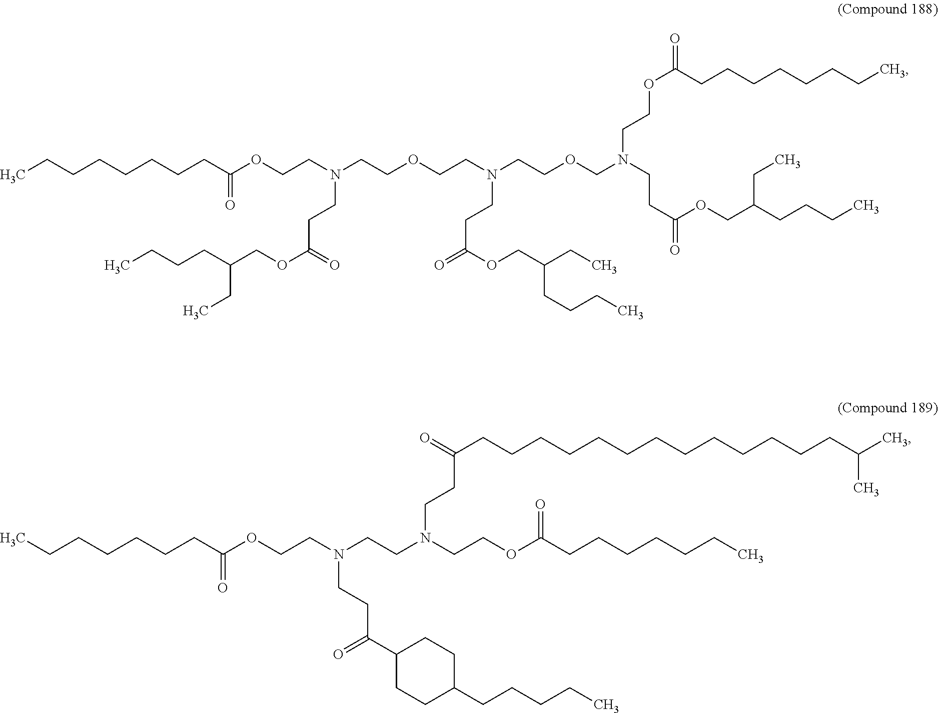 [SMILES: CCCCCCCCC(=O)OCCN(CCOCCN(CCOCN(CCOC(=O)CCCCCCCC)CCC(=O)OCC(CC)CCCC)CCC(=O)OCC(CC)CCCC)CCC(=O)OCC(CC)CCCC ; CCCCCCCC(=O)OCCN(CCC(=O)CCCCCCCCCCCCCC(C)C)CCN(CCOC(=O)CCCCCCC)CCC(=O)C1CCC(CCCCC)CC1]

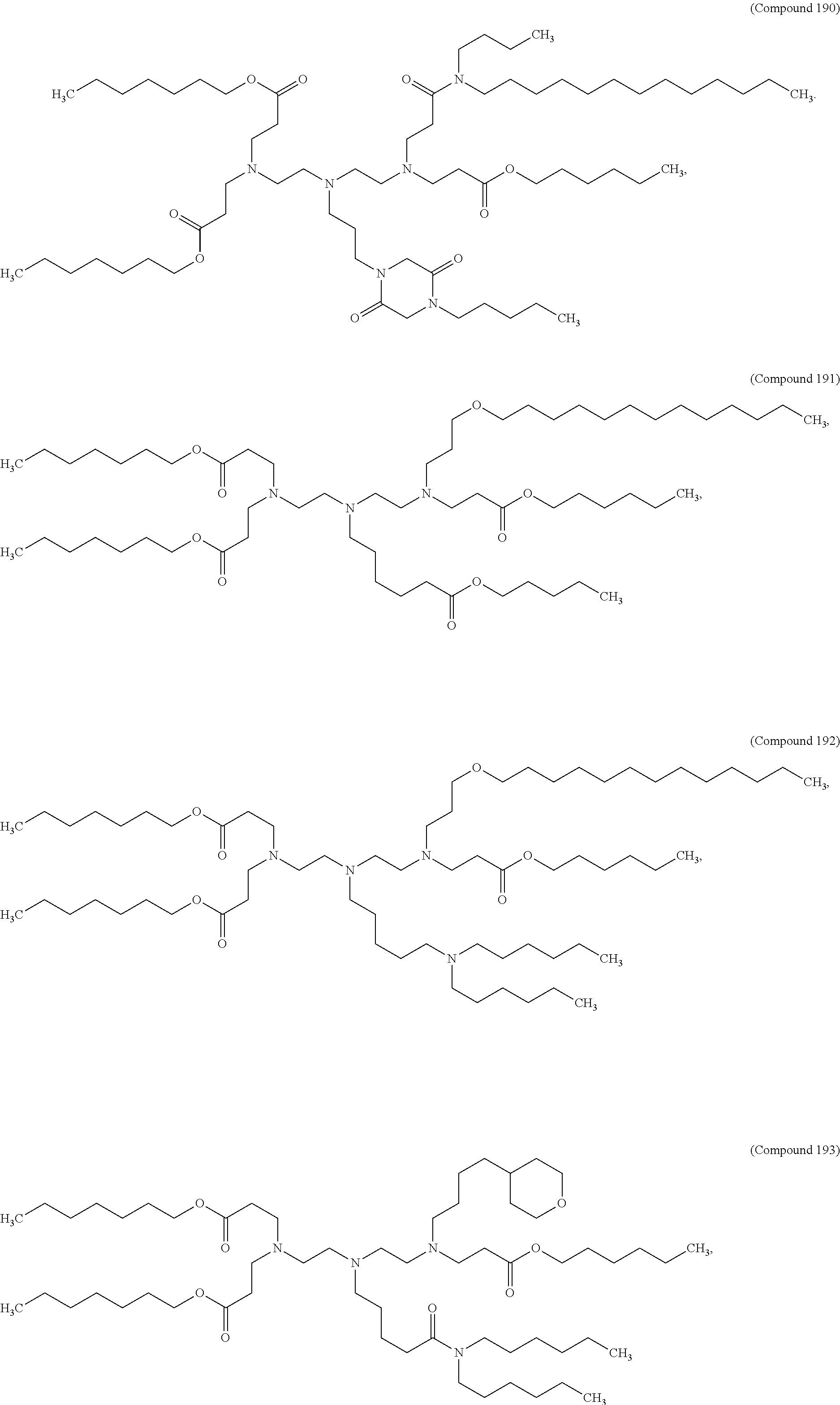 [SMILES: CCCCCCCCCCCCCN(CCCC)C(=O)CCN(CCC(=O)OCCCCCC)CCN(CCCN1CC(=O)N(CCCCC)CC1=O)CCN(CCC(=O)OCCCCCCC)CCC(=O)OCCCCCCC ; CCCCCCCCCCCCCOCCCN(CCC(=O)OCCCCCC)CCN(CCCCCC(=O)OCCCCC)CCN(CCC(=O)OCCCCCCC)CCC(=O)OCCCCCCC ; CCCCCCCCCCCCCOCCCN(CCC(=O)OCCCCCC)CCN(CCCCCN(CCCCCC)CCCCCC)CCN(CCC(=O)OCCCCCCC)CCC(=O)OCCCCCCC ; CCCCCCCOC(=O)CCN(CCC(=O)OCCCCCCC)CCN(CCCCC(=O)N(CCCCCC)CCCCCC)CCN(CCCCC1CCOCC1)CCC(=O)OCCCCCC]

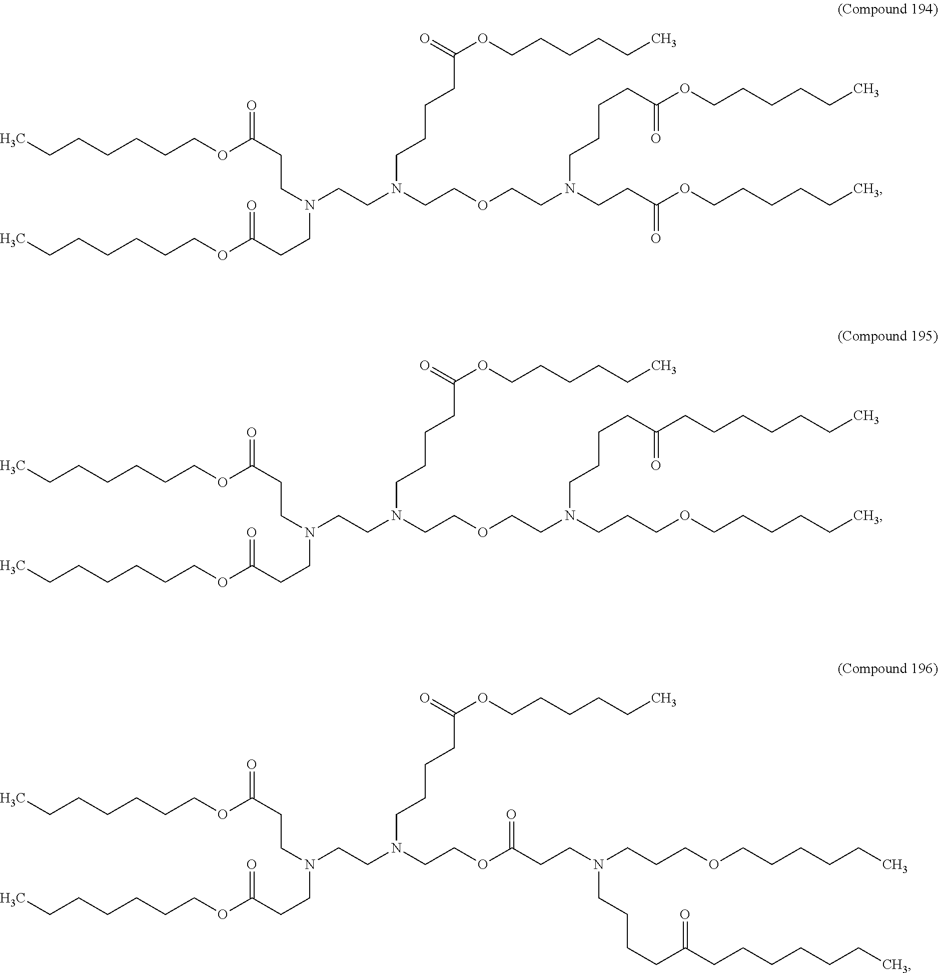

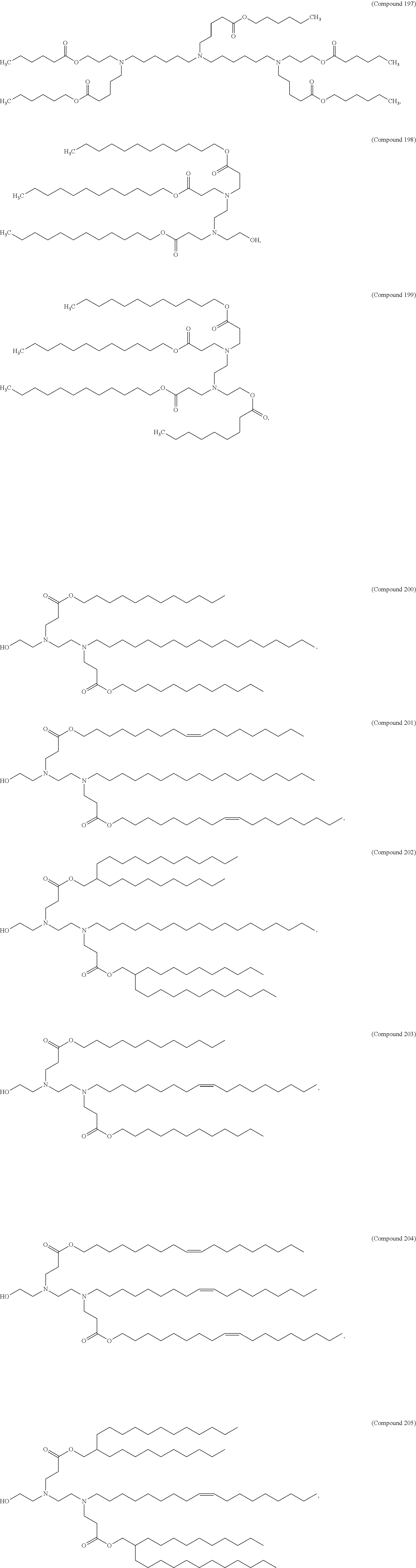

or salts or isomers thereof.

In another aspect of the disclosure, the compounds described herein are of Formula V or a salt or isomer thereof

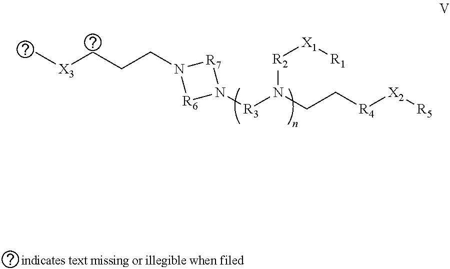

wherein:
R₁ is a C₂₋₁₈ alkyl, C₂₋₁₈ alkenyl, or C₂₋₁₈ alkynyl, a C₅₋₈ carbocyclyl, a heterocyclyl, hydrogen, or absent;
R₂ is a C₁₋₁₈ alkylene, C₂₋₁₈ alkenylene, or C₂₋₁₈ alkynylene;
R₃ is a C₁₋₁₈ alkylene, C₂₋₁₈ alkenylene, or C₂₋₁₈ alkynylene, each optionally substituted by one, two, or three substituents independently selected from —OR, —SR, —S—S—R, —O—CO—R, —CO—OR, —CO—NR^{a}R^{b}, —NR^{a}—CO—R, —O—CO—N^{a}R^{b}, —NR^{a}—CO—OR, —N^{a}R^{b}, and —S—CO—R, wherein R at each occurrence is independently hydrogen or lower alkyl; and R^{a} and R^{b} are independently hydrogen or lower alkyl;
n is 0, 1, 2, 3, 4, 5, 6, 7, 8, 9, or 10;
R₄ and R₈ are the same or different, each independently a bond or a C₁₋₂₈ alkylene, C₂₋₂₈ alkenylene, or C₂₋₂₈ alkynylene;
R₅ and R₉ are the same or different, each independently hydrogen or a C₁₋₂₈ alkyl, C₂₋₂₈ alkenyl, or C₂₋₂₈ alkynyl;
R₆ and R₇ are the same or different, each independently a bond or a C₁₋₁₈ alkylene, C₂₋₁₈ alkenylene, or C₂₋₁₈ alkynylene;
X₁ is a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent; and
X₂, and X₃ are the same or different, each independently a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent,
wherein the longest chain of atoms in the compound is between 18 to 70 atoms.

In some embodiments, R₂ and X₁ together with the atoms they are attached be part of a 3- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₃ may together with the one or both atoms it is attached, form a part of a 3- to 10-membered heterocyclic ring having one or more of the heteroatoms selected from N, O, S, or P, which can be optionally part of a functional group such as, but not limited to ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₆ and R₇ are optionally substituted with one or more substituents, such as, but not limited to, alkyl groups, alkenyl groups, alkynyl groups, C₃₋₇ carbocyclic groups, C₃₋₇ heterocyclic groups, —OH, —SH, —OR, —SR, —NR₂, NHR, -oxo, —O—CO—, —NR—CO—, or other functional groups.

In some embodiments, R₄ and X₂ may together with the atoms they are attached form a part of a C₃₋₁₀ carbocyclic or 3- to 10-membered heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₈ and X₃ may together with the atoms they are attached form a part of a 3- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the ring may be optionally substituted with one or more substituents, such as, but not limited to, alkyl groups, alkenyl groups, alkynyl groups, C₃₋₇ carbocyclic groups, 3- to 8-membered heterocyclic groups —OH, —SH, —OR, —SR, —NR₂, NHR, -oxo, —O—CO—, —NR—CO—, or other functional groups.

In some embodiments, n is 3.

In some embodiments, R₃ is methyl, ethyl, propyl, butyl, methyoxymethyl, ethoxyethyl, or methoxyethyl.

In some embodiments, R₂ consists of an ethyl.

In some embodiments, X₁ is —O—CO— or —CO—O—.

In some embodiments, R₁ a C₁₀ alkyl.

In some embodiments, R₆ and R₇ are each independently ethylene, propylene, or butylene.

In some embodiments, X₂ and X₃ are independently —O—CO— or —CO—O—.

In some embodiments, in the compounds of Formula V:
R₁ is a C₂₋₈ alkyl, C₂₋₈ alkenyl, or C₂₋₈ alkynyl, a C₅₋₈ carbocyclyl, a heterocyclyl, or hydrogen;
R₂ is a C₁₋₈ alkylene, C₂₋₈ alkenylene, or C₂₋₈ alkynylene;
R₃ is a C₁₋₈ alkylene, C₂₋₈ alkenylene, or C₂₋₈ alkynylene, each optionally substituted by one, two, or three substituents independently selected from —OR, —O—CO—R, —CO—OR, and —NR^{a}R^{b}, wherein R at each occurrence is independently hydrogen or lower alkyl; and R^{a} and R^{b} are independently hydrogen or lower alkyl;
n is 0, 1, 2, 3, 4, or 5;
R₄ and R₈ are the same or different, each independently a bond or a C₁₋₁₄ alkylene, C₂₋₁₄ alkenylene, or C₂₋₁₄ alkynylene;
R₅ and R₉ are the same or different, each independently hydrogen or a C₁₋₁₄ alkyl, C₂₋₁₄ alkenyl, or C₂₋₁₄ alkynyl;
R₆ and R₇ are the same or different, each independently a bond or a C₁-s alkylene, C₂₋₈ alkenylene, or C₂₋₈ alkynylene;
X₁ is a methylene (—CH₂—), —O—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, or a bond; and
X₂, and X₃ are the same or different, each independently a methylene (—CH₂—), —O—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, or a bond,
wherein the longest chain of atoms in the compound is between 18 to 70 atoms.

In some embodiments, the compound of Formula V may be selected from the Compounds of List 5:

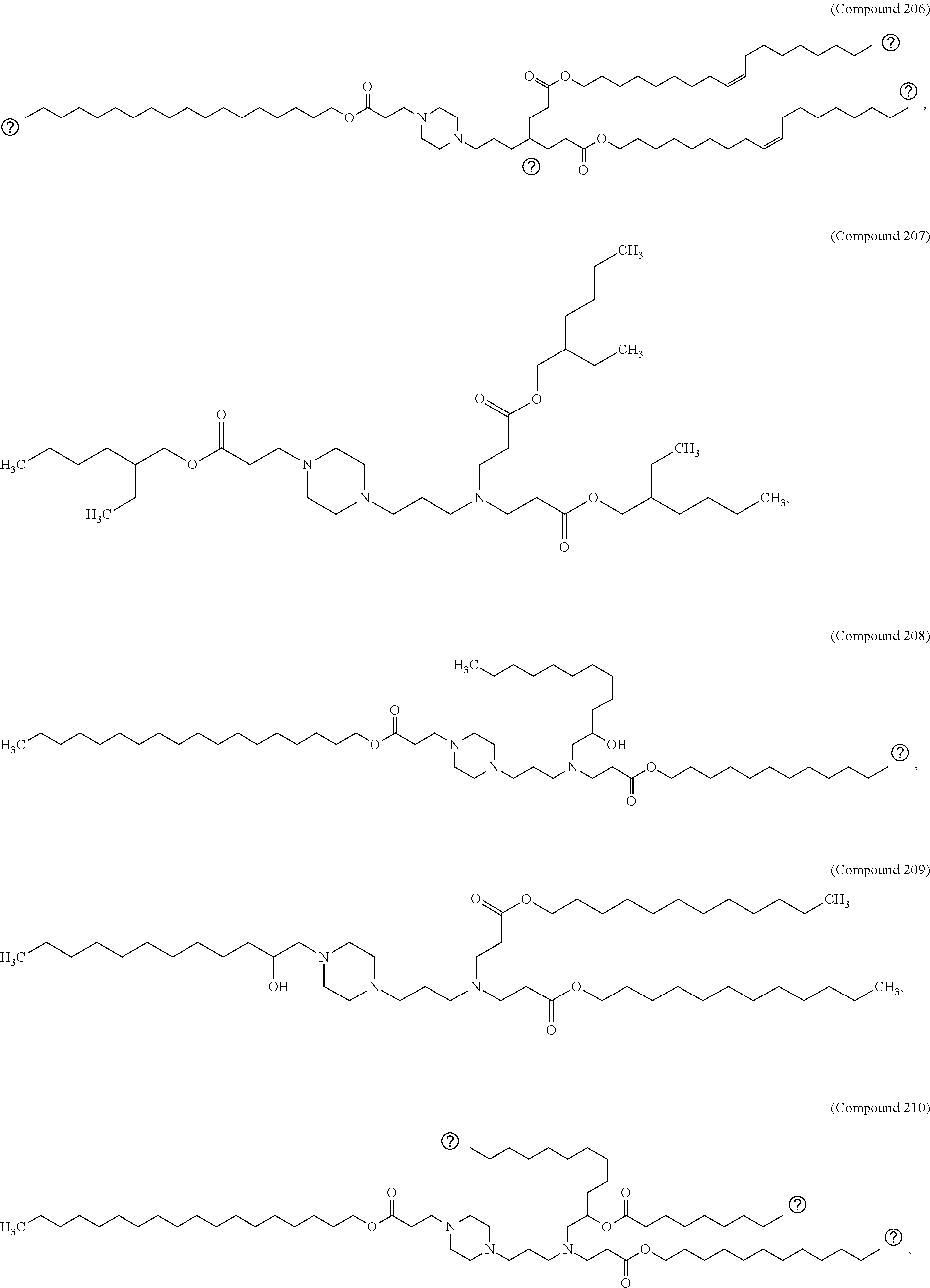

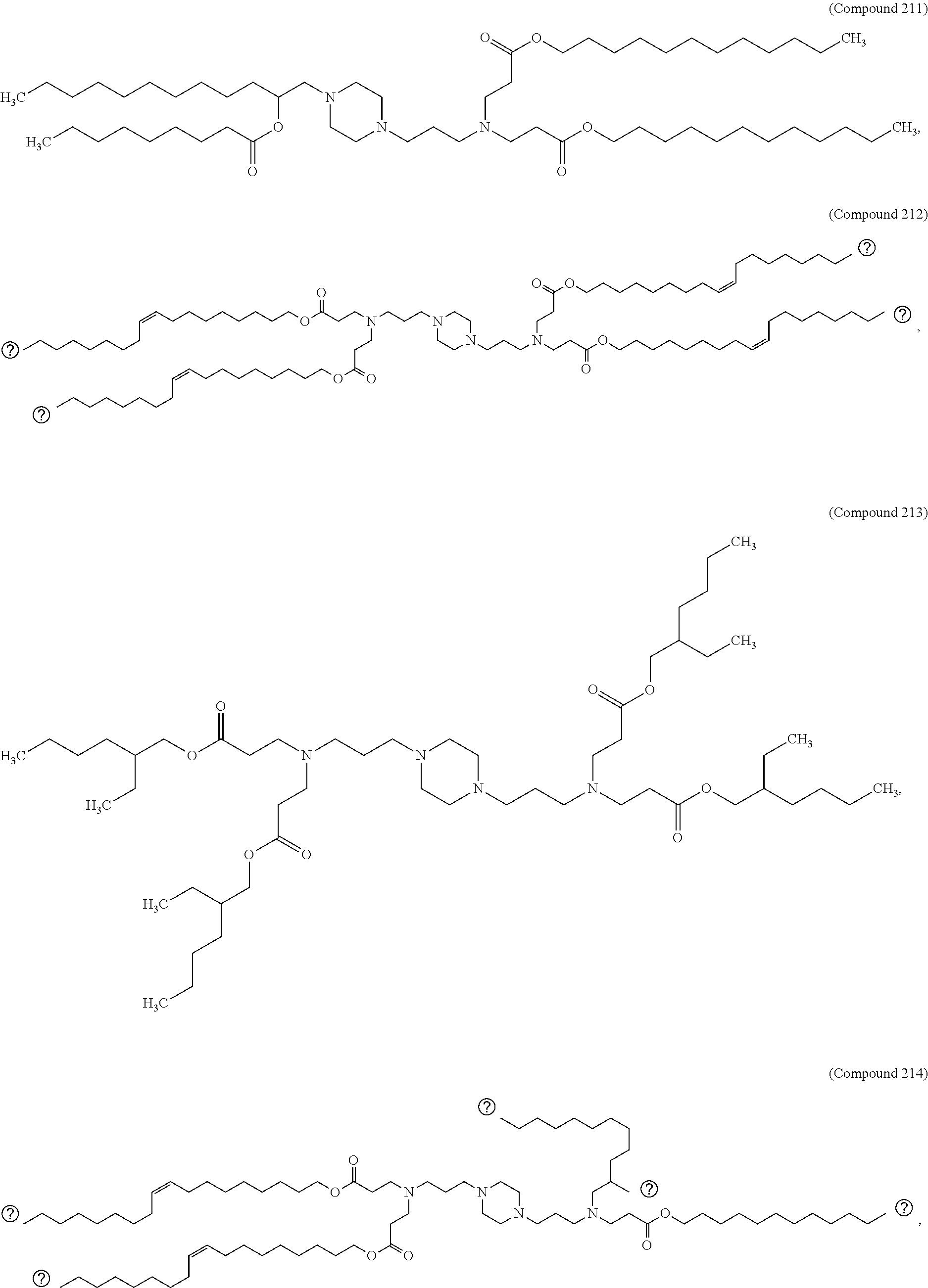

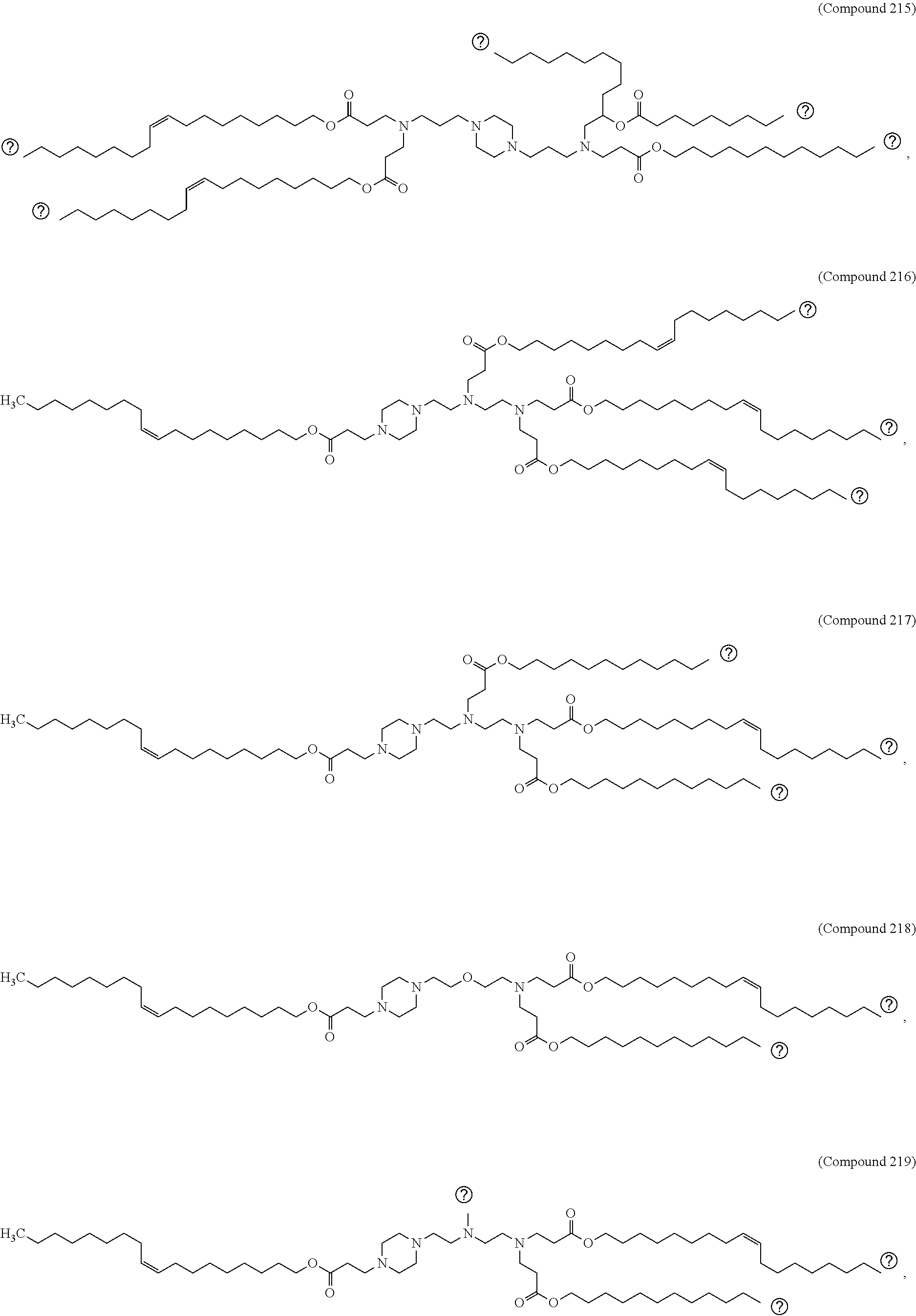

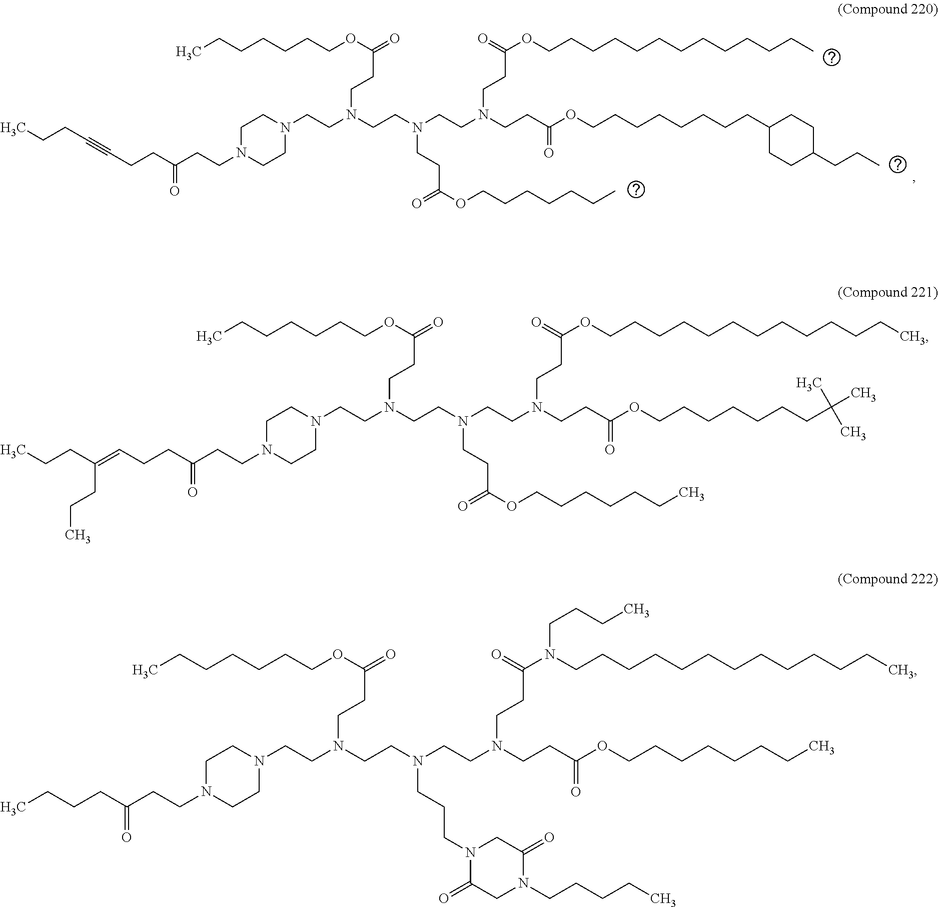

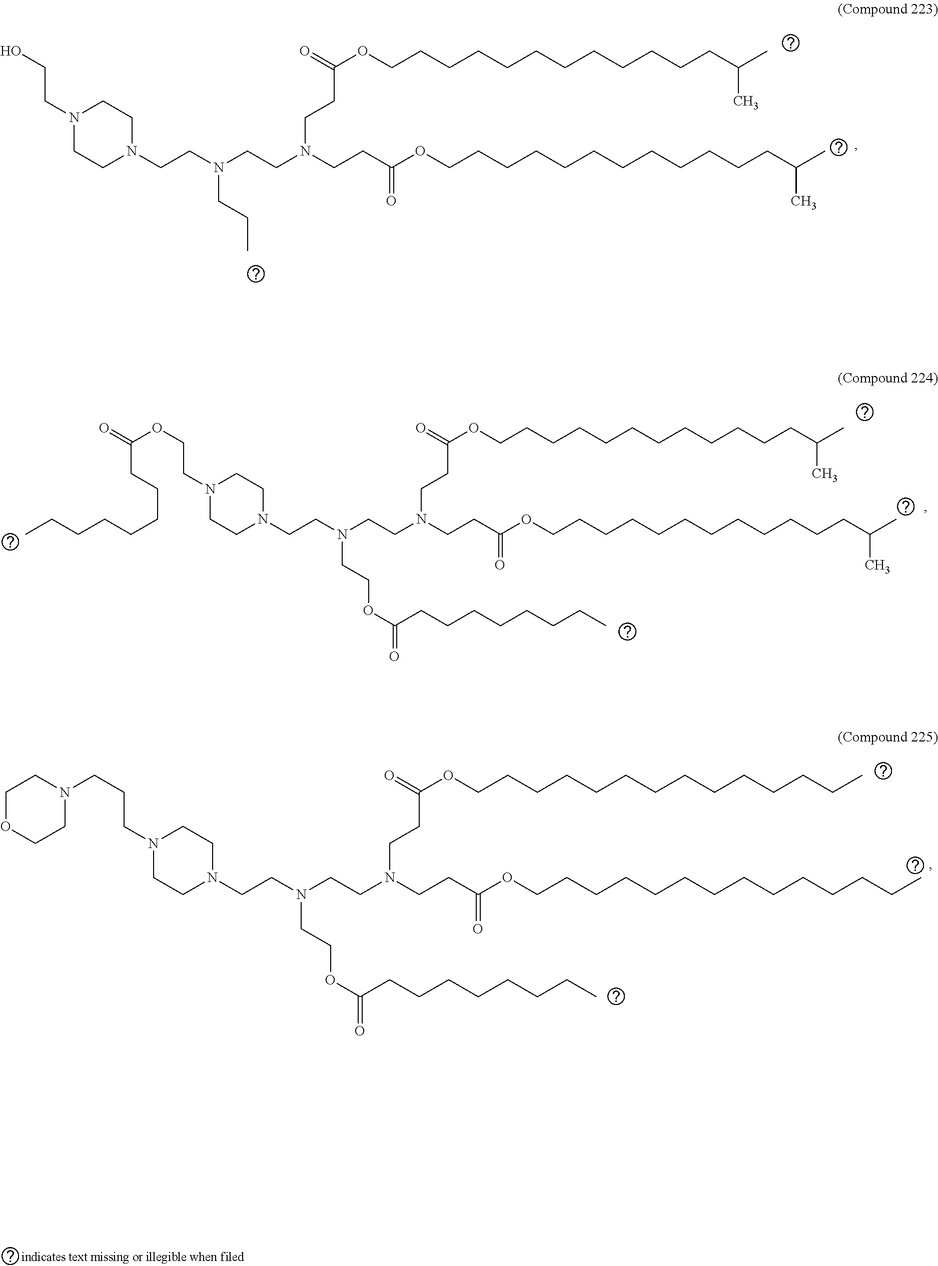

or salts or isomers thereof.

In one aspect of the disclosure, the compounds described herein are of Formula VI or a salt or isomer thereof

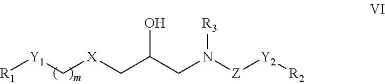

wherein:

Y₁ and Y₂ are the same or different, each independently selected from —(C═O)O—, —O(C═O)—, —(C═O)S—, —S(C═O)—, —NR(C═O)—, —(C═O)NR—, —O—, —S—, —S—S—, —CH₂—, an alkenylene, and an alkynylene, wherein R is independently a hydrogen, or an alkyl, alkenyl, alkynyl, carbocyclyl, heterocyclyl, group;
X is selected from —S—, —S—S—, —O—, —CH₂—, alkenylene, and alkynylene, —NR—, wherein R is hydrogen or an alkyl, alkenyl, alkynyl, carbocyclyl, heterocyclyl, group;
R₁ and R₂ are the same or different, each independently selected from carbocyclyl, alkyl, alkenyl, and alkynyl, each optionally substituted;
R₃ is selected from hydrogen, halogen, alkyl, alkenyl, alkynyl, carbocyclyl, heterocyclyl, aryl, and heteroaryl, each, except hydrogen and halogen, optionally substituted;
Z is selected from alkylene, alkenylene, alkynylene, and carbocyclylene;
m is 1 to 24.
wherein the longest chain of atoms in the compound is between 18 to 70 atoms.

In some embodiments, R₂, Y₂, and R₃ may together form part of a 4- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the carbocyclic or heterocyclic ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, X, Y₁, and R₁ may together form part of a 4- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the carbocyclic or heterocyclic ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, Y₁, X, and Y₂ may together form part of a 5- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the carbocyclic or heterocyclic ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, Y₁ and Y₂ are independently selected from —(C═O)O—, —O(C═O)—, —(C═O)S—, —S(C═O)—, —NR(C═O)—, and —(C═O)NR—.

In some embodiments, R₃ is selected from or an alkyl, alkenyl, alkynyl, carbocyclyl, heterocyclyl, aryl, or heteroaryl, each optionally substituted by —OH.

In some embodiments, X is oxygen (—O—) or substituted or unsubstituted nitrogen (—NR—).

In some embodiments, in the compounds of Formula VI:
Y₁ and Y₂ are the same or different, each independently selected from —(C═O)O—, —O(C═O)—, —NR(C═O)—, —(C═O)NR—, —O—, —CH₂—, an alkenylene, and an alkynylene, wherein R is independently a hydrogen, or an alkyl, alkenyl, alkynyl, carbocyclyl, heterocyclyl, group;
X is selected from —S—, —O—, —CH₂—, alkenylene, and alkynylene, —NR—, wherein R is hydrogen or an alkyl, alkenyl, alkynyl, carbocyclyl, heterocyclyl, group;
R₁ and R₂ are the same or different, each independently selected from carbocyclyl, alkyl, alkenyl, and alkynyl, each optionally substituted;
R₃ is selected from hydrogen, alkyl, alkenyl, alkynyl, carbocyclyl, heterocyclyl, aryl, and heteroaryl, each, except hydrogen, optionally substituted;
Z is selected from alkylene, alkenylene, alkynylene, and carbocyclylene; and
m is 1 to 14,
wherein the longest chain of atoms in the compound is between 18 to 70 atoms.

In some embodiments, the compound of Formula VI may be selected from the Compounds of List 6:

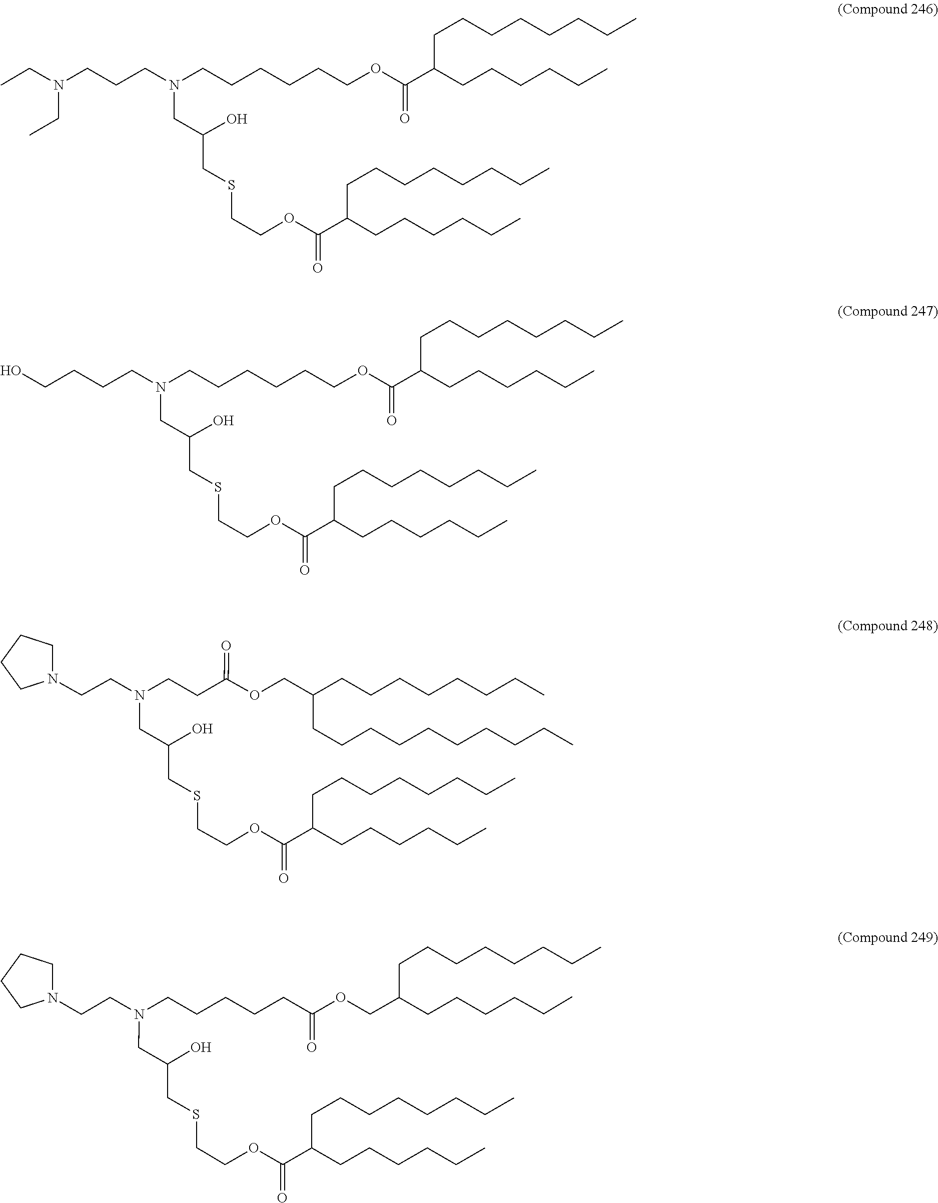 [SMILES: CCCCCCCCC(CCCCCC)C(=O)OCCCCCCN(CCCN(CC)CC)CC(O)CSCCOC(=O)C(CCCCCC)CCCCCCCC ; CCCCCCCCC(CCCCCC)C(=O)OCCCCCCN(CCCCO)CC(O)CSCCOC(=O)C(CCCCCC)CCCCCCCC ; CCCCCCCCCCC(CCCCCCCC)COC(=O)CCN(CCN1CCCC1)CC(O)CSCCOC(=O)C(CCCCCC)CCCCCCCC ; CCCCCCCCC(CCCCCC)COC(=O)CCCCCN(CCN1CCCC1)CC(O)CSCCOC(=O)C(CCCCCC)CCCCCCCC]

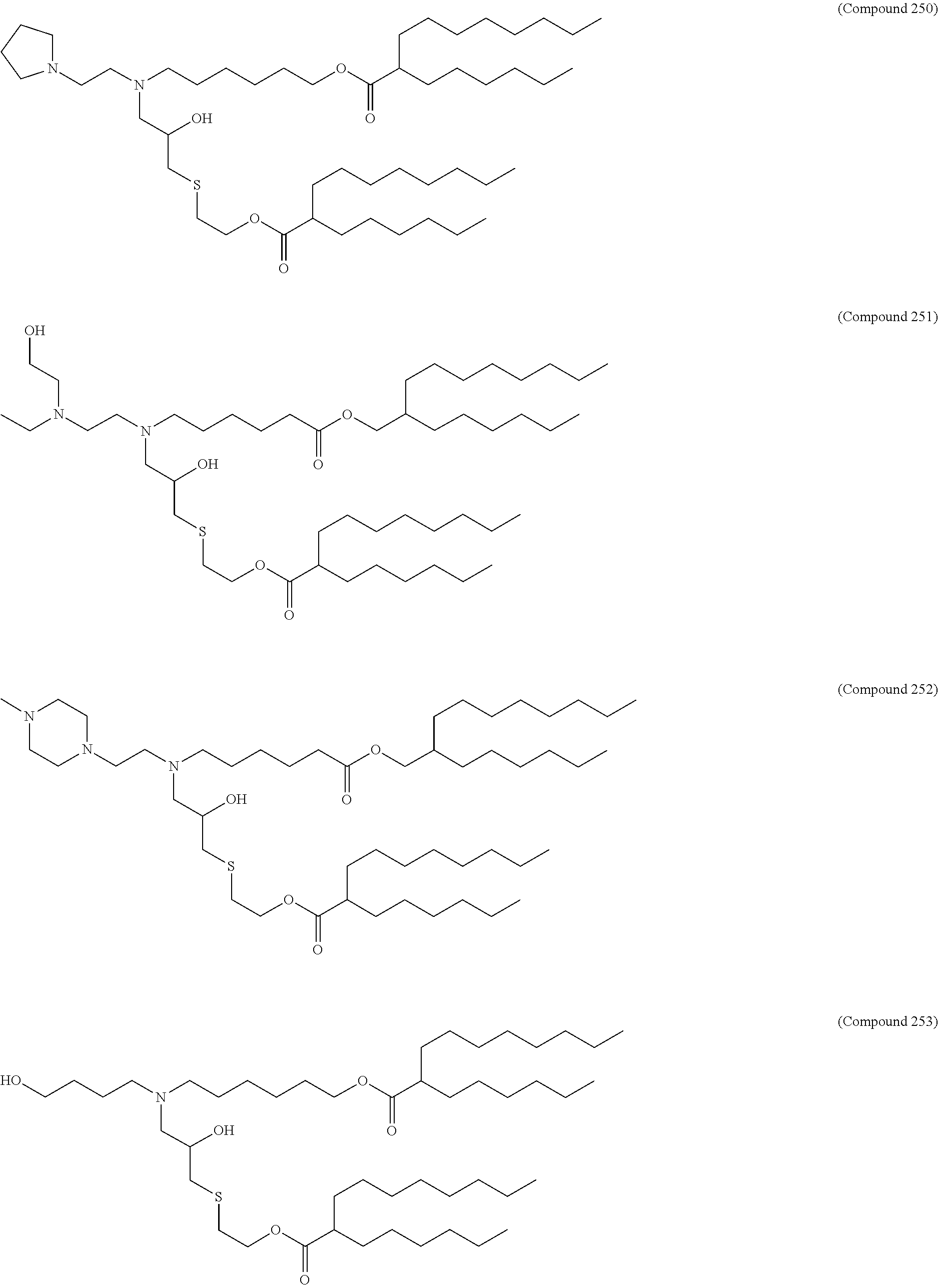

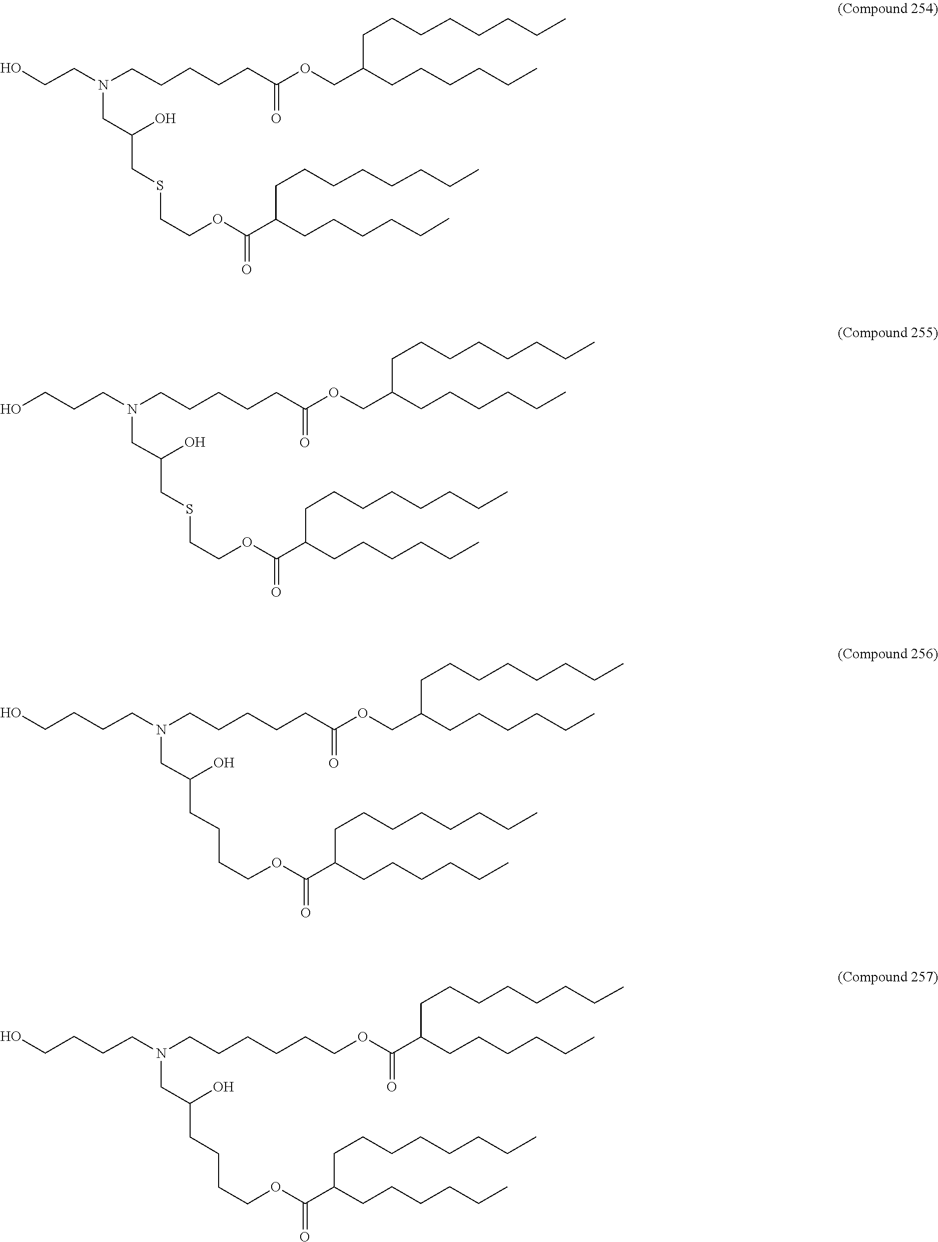 [SMILES: CCCCCCCCC(CCCCCC)COC(=O)CCCCCN(CCO)CC(O)CSCCOC(=O)C(CCCCCC)CCCCCCCC ; CCCCCCCCC(CCCCCC)COC(=O)CCCCCN(CCCO)CC(O)CSCCOC(=O)C(CCCCCC)CCCCCCCC ; CCCCCCCCC(CCCCCC)COC(=O)CCCCCN(CCCCO)CC(O)CCCCOC(=O)C(CCCCCC)CCCCCCCC ; CCCCCCCCC(CCCCCC)C(=O)OCCCCCCN(CCCCO)CC(O)CCCCOC(=O)C(CCCCCC)CCCCCCCC]

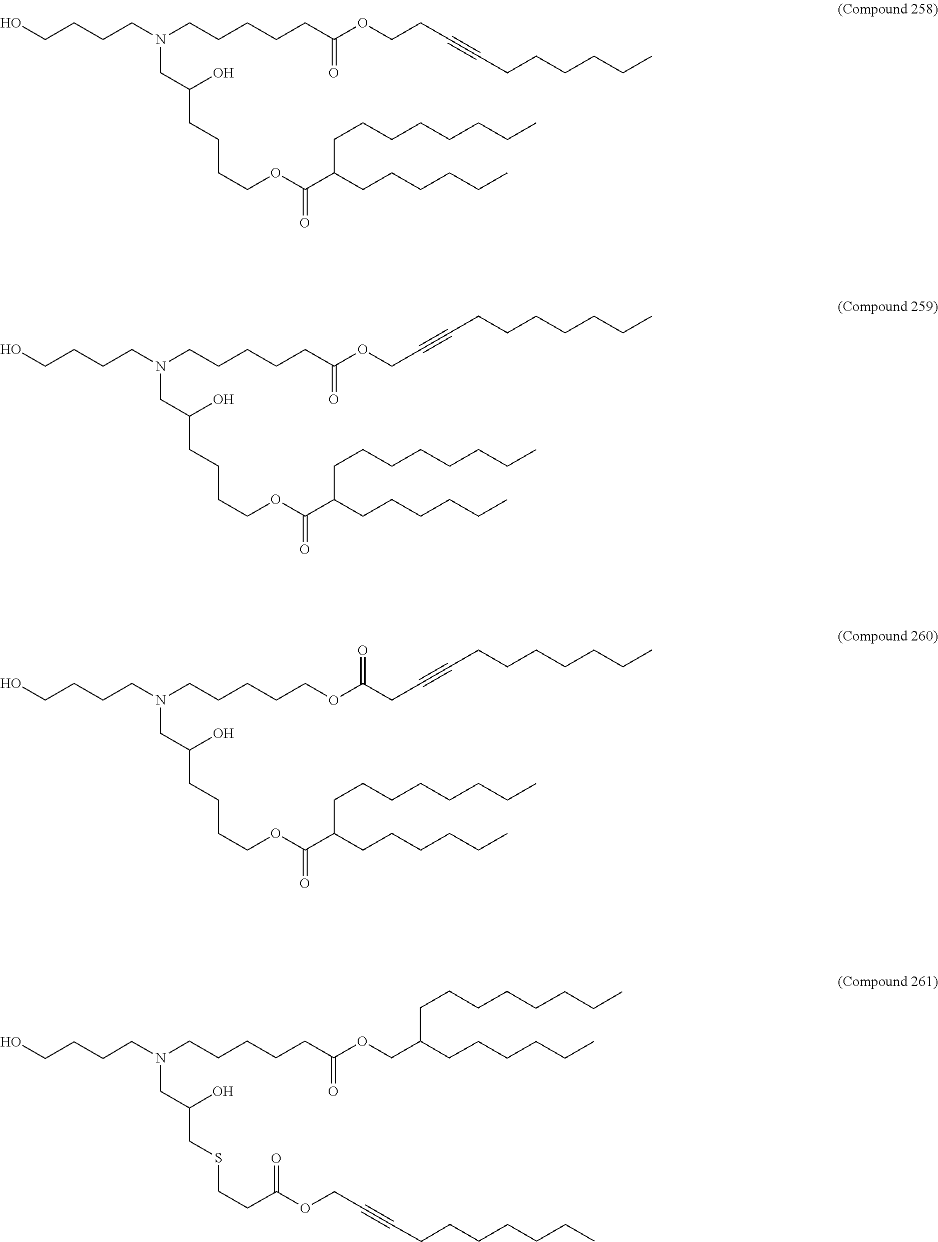 [SMILES: CCCCCCC#CCCOC(=O)CCCCCN(CCCCO)CC(O)CCCCOC(=O)C(CCCCCC)CCCCCCCC ; CCCCCCCC#CCOC(=O)CCCCCN(CCCCO)CC(O)CCCCOC(=O)C(CCCCCC)CCCCCCCC ; CCCCCCCC#CCC(=O)OCCCCCN(CCCCO)CC(O)CCCCOC(=O)C(CCCCCC)CCCCCCCC ; CCCCCCCC#CCOC(=O)CCSCC(O)CN(CCCCO)CCCCCC(=O)OCC(CCCCCC)CCCCCCCC]

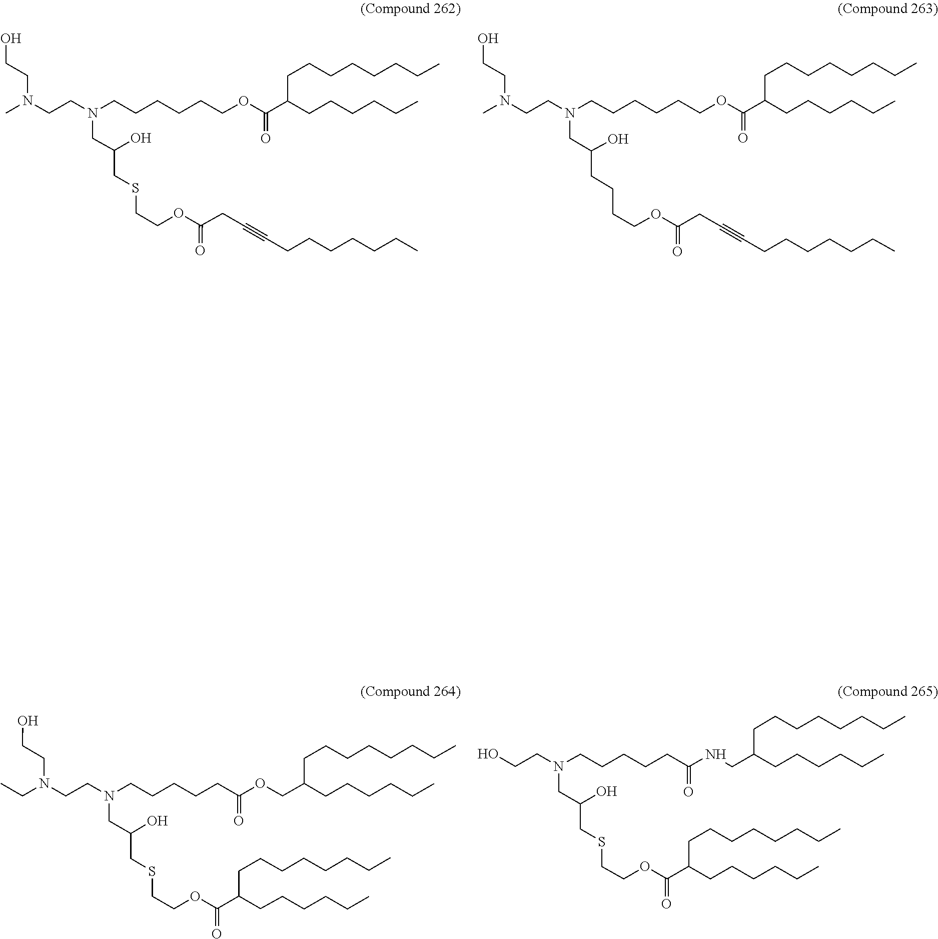 [SMILES: CCCCCCCC#CCC(=O)OCCSCC(O)CN(CCCCCCOC(=O)C(CCCCCC)CCCCCCCC)CCN(C)CCO ; CCCCCCCC#CCC(=O)OCCCCC(O)CN(CCCCCCOC(=O)C(CCCCCC)CCCCCCCC)CCN(C)CCO ; CCCCCCCCC(CCCCCC)COC(=O)CCCCCN(CCN(CC)CCO)CC(O)CSCCOC(=O)C(CCCCCC)CCCCCCCC ; CCCCCCCCC(CCCCCC)CNC(=O)CCCCCN(CCO)CC(O)CSCCOC(=O)C(CCCCCC)CCCCCCCC]

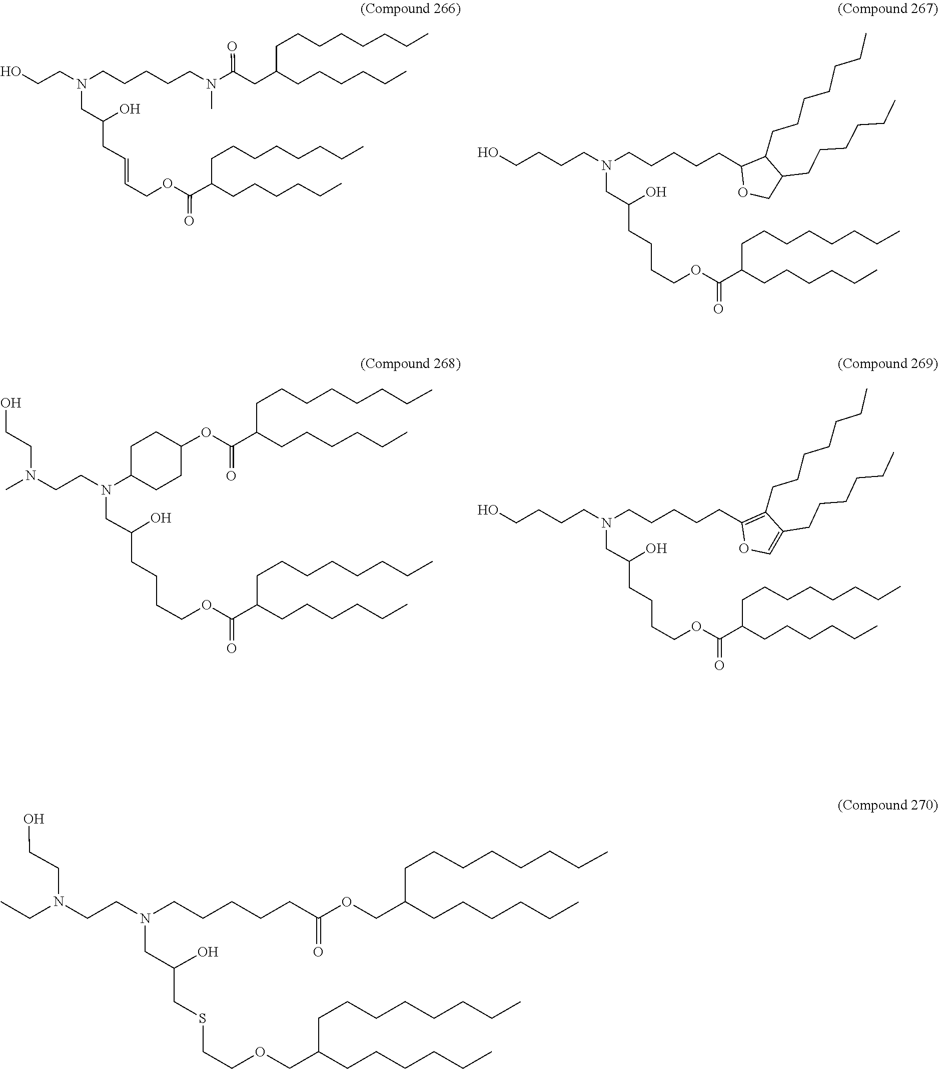 [SMILES: CCCCCCCCC(CCCCCC)CC(=O)N(C)CCCCCN(CCO)CC(O)C/C=C/COC(=O)C(CCCCCC)CCCCCCCC ; CCCCCCCCC(CCCCCC)C(=O)OCCCCC(O)CN(CCCCO)CCCCCC1OCC(CCCCCC)C1CCCCCCC ; CCCCCCCCC(CCCCCC)C(=O)OCCCCC(O)CN(CCN(C)CCO)C1CCC(OC(=O)C(CCCCCC)CCCCCCCC)CC1 ; CCCCCCCCC(CCCCCC)C(=O)OCCCCC(O)CN(CCCCO)CCCCCc1occ(CCCCCC)c1CCCCCCC ; CCCCCCCCC(CCCCCC)COCCSCC(O)CN(CCCCCC(=O)OCC(CCCCCC)CCCCCCCC)CCN(CC)CCO]

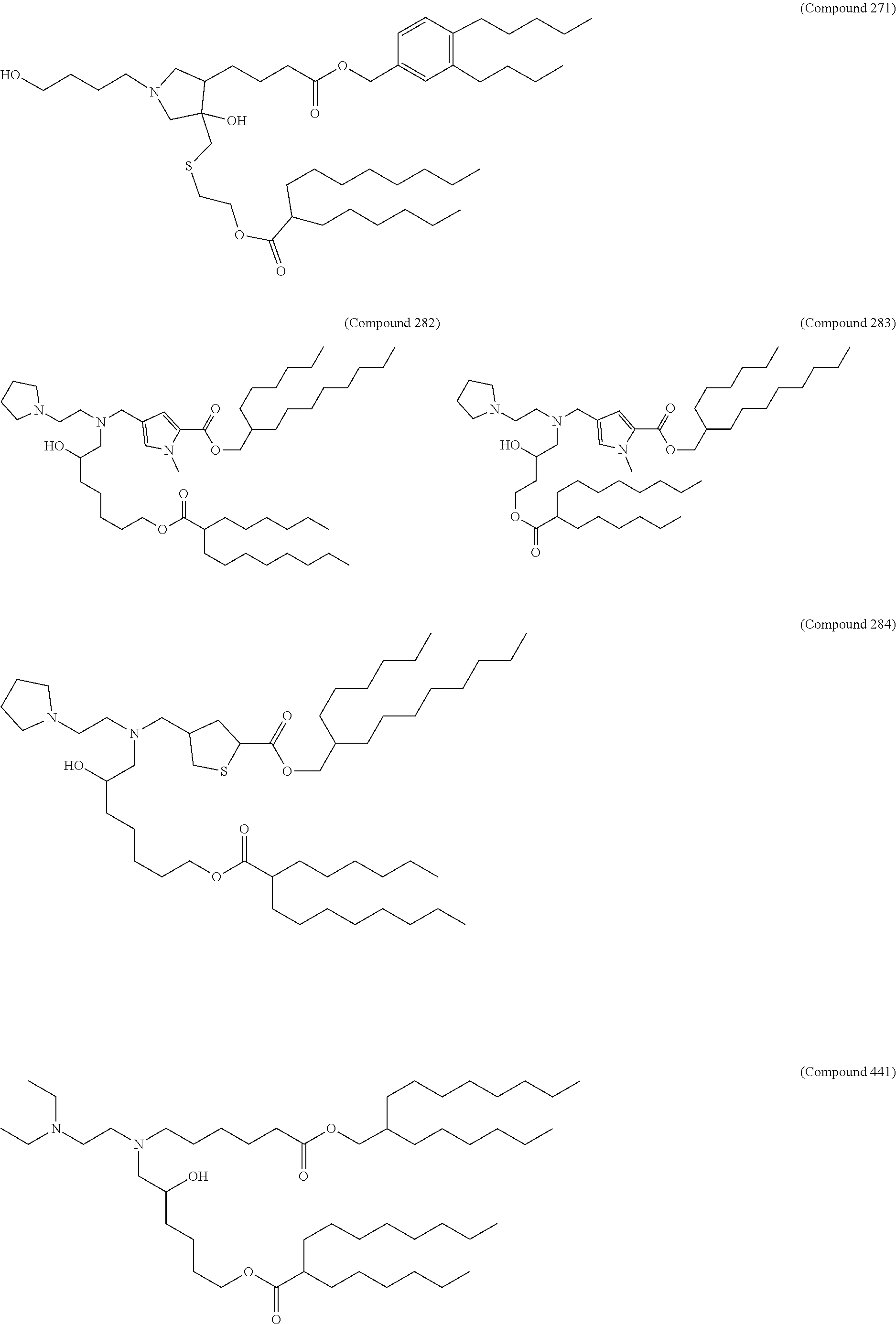

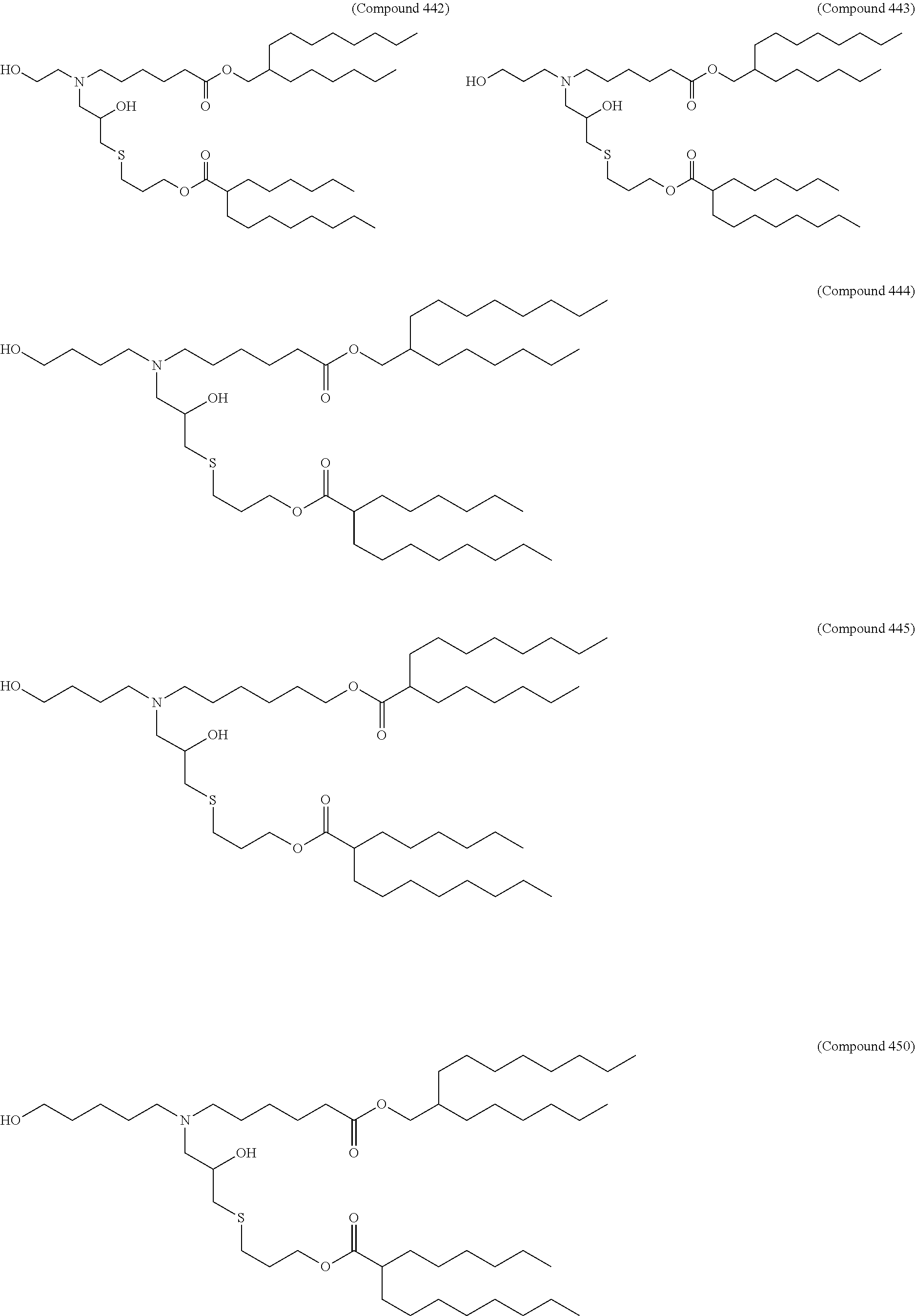 [SMILES: CCCCCCCCC(CCCCCC)COC(=O)CCCCCN(CCO)CC(O)CSCCCOC(=O)C(CCCCCC)CCCCCCCC ; CCCCCCCCC(CCCCCC)COC(=O)CCCCCN(CCCO)CC(O)CSCCCOC(=O)C(CCCCCC)CCCCCCCC ; CCCCCCCCC(CCCCCC)COC(=O)CCCCCN(CCCCO)CC(O)CSCCCOC(=O)C(CCCCCC)CCCCCCCC ; CCCCCCCCC(CCCCCC)C(=O)OCCCCCCN(CCCCO)CC(O)CSCCCOC(=O)C(CCCCCC)CCCCCCCC ; CCCCCCCCC(CCCCCC)COC(=O)CCCCCN(CCCCCO)CC(O)CSCCCOC(=O)C(CCCCCC)CCCCCCCC]

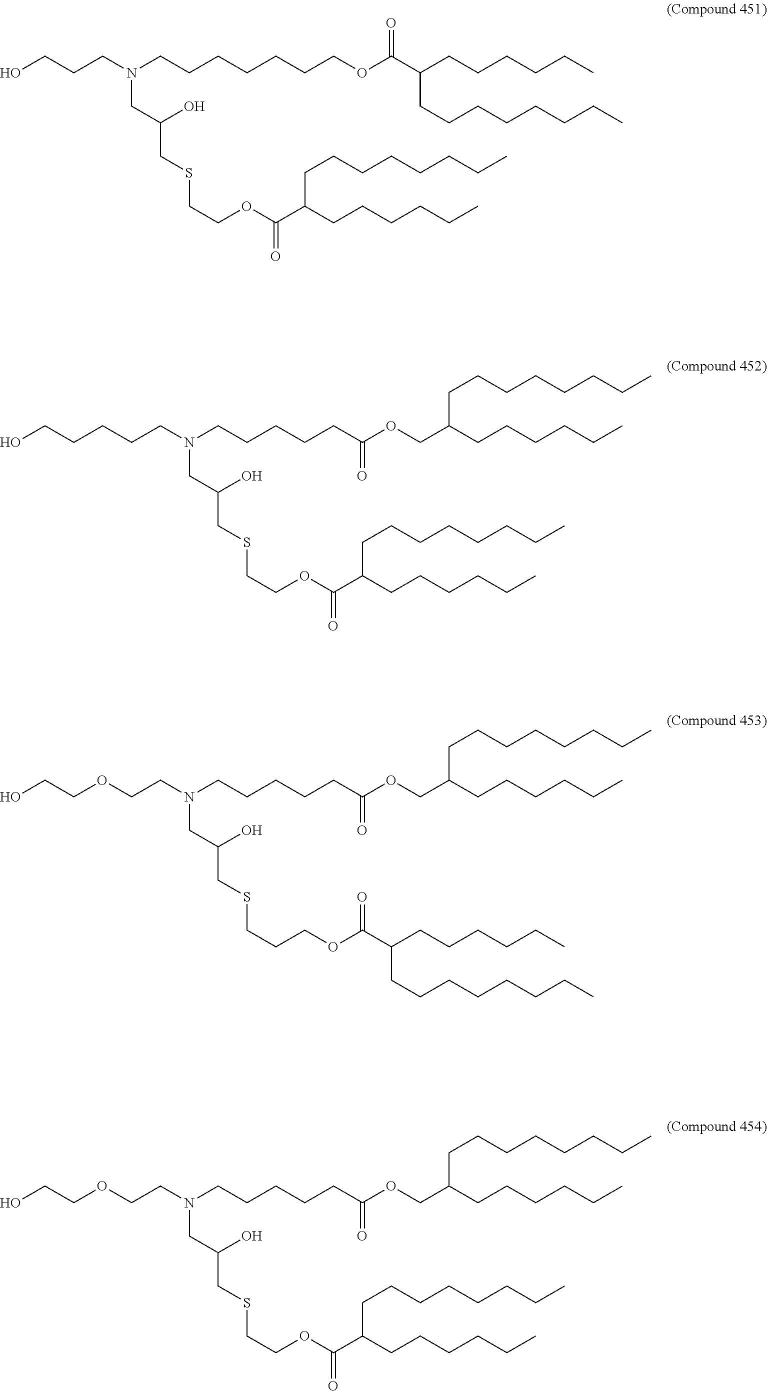 [SMILES: CCCCCCCCC(CCCCCC)C(=O)OCCCCCCCN(CCCO)CC(O)CSCCOC(=O)C(CCCCCC)CCCCCCCC ; CCCCCCCCC(CCCCCC)COC(=O)CCCCCN(CCCCCO)CC(O)CSCCOC(=O)C(CCCCCC)CCCCCCCC ; CCCCCCCCC(CCCCCC)COC(=O)CCCCCN(CCOCCO)CC(O)CSCCCOC(=O)C(CCCCCC)CCCCCCCC ; CCCCCCCCC(CCCCCC)COC(=O)CCCCCN(CCOCCO)CC(O)CSCCOC(=O)C(CCCCCC)CCCCCCCC]

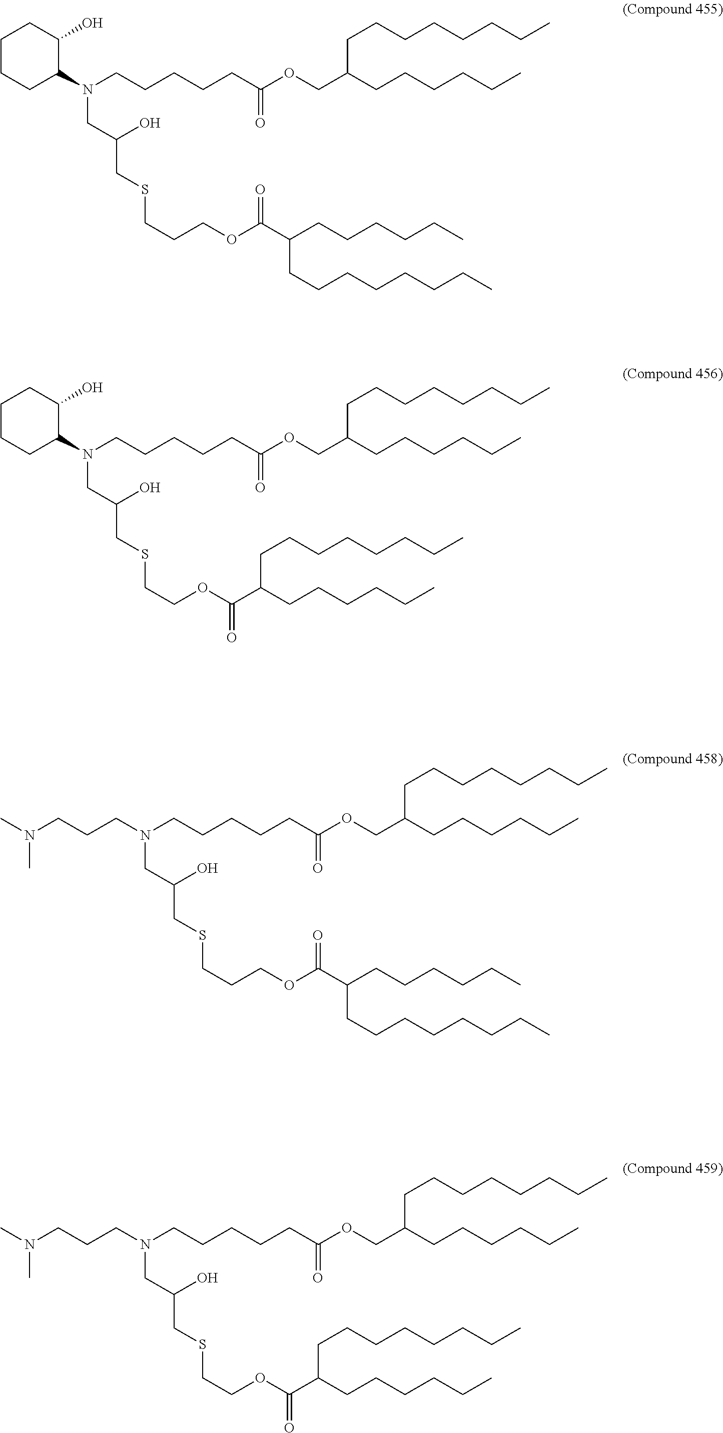

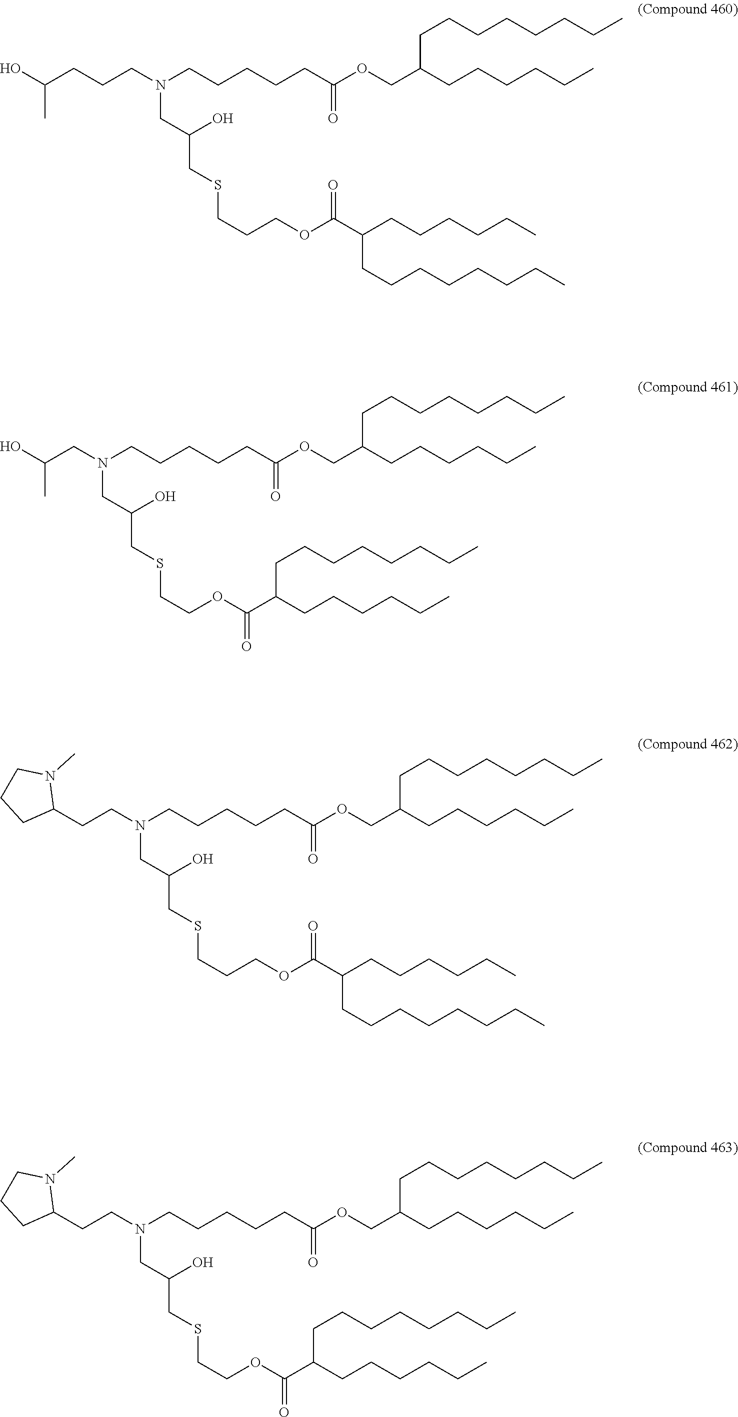 [SMILES: CCCCCCCCC(CCCCCC)COC(=O)CCCCCN(CCCC(C)O)CC(O)CSCCCOC(=O)C(CCCCCC)CCCCCCCC ; CCCCCCCCC(CCCCCC)COC(=O)CCCCCN(CC(C)O)CC(O)CSCCOC(=O)C(CCCCCC)CCCCCCCC ; CCCCCCCCC(CCCCCC)COC(=O)CCCCCN(CCC1CCCN1C)CC(O)CSCCCOC(=O)C(CCCCCC)CCCCCCCC ; CCCCCCCCC(CCCCCC)COC(=O)CCCCCN(CCC1CCCN1C)CC(O)CSCCOC(=O)C(CCCCCC)CCCCCCCC]

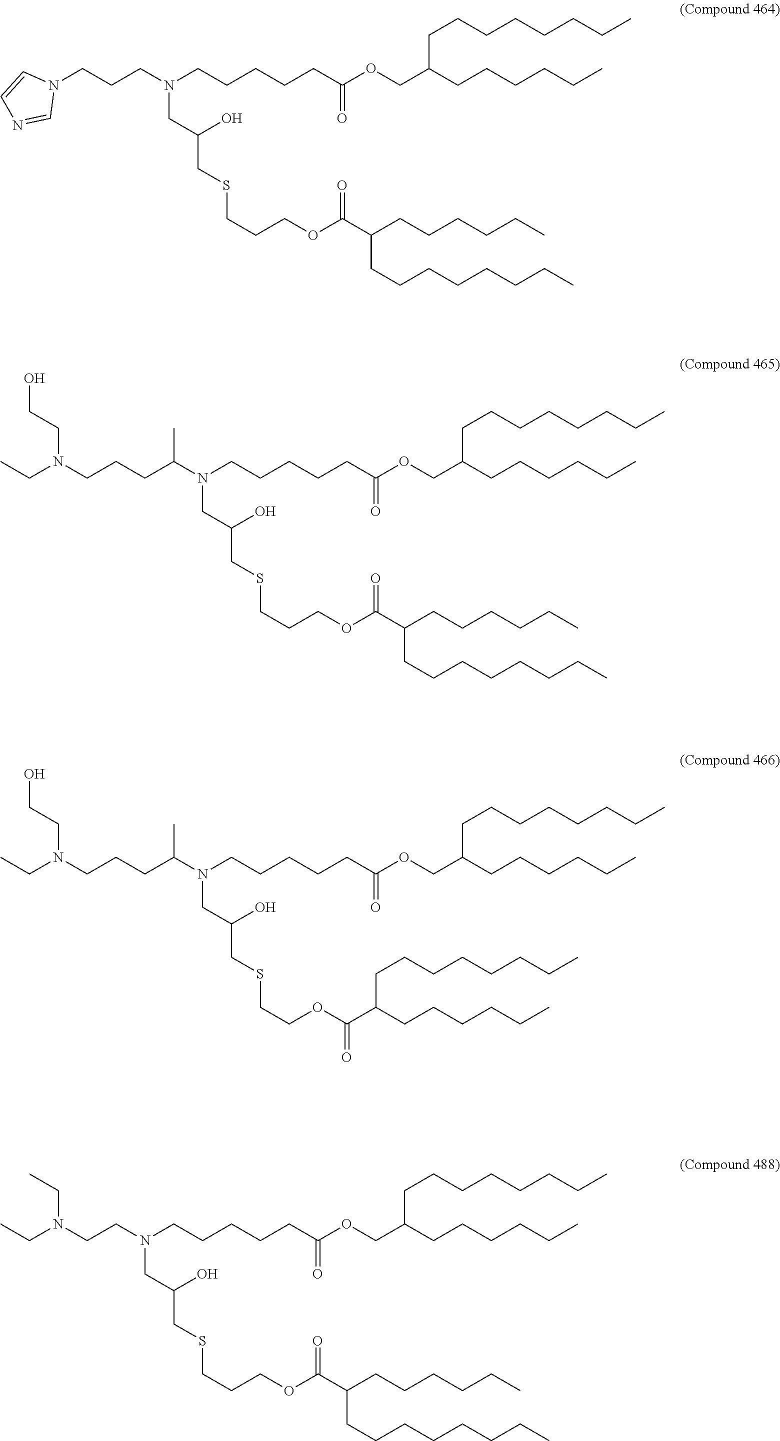

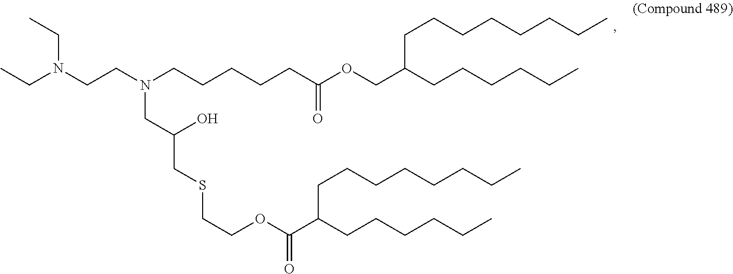 [SMILES: CCCCCCCCC(CCCCCC)COC(=O)CCCCCN(CCN(CC)CC)CC(O)CSCCOC(=O)C(CCCCCC)CCCCCCCC]

and salts and isomers thereof.

In another aspect of the disclosure, the compounds described herein are of Formula VII or a salt or isomer thereof

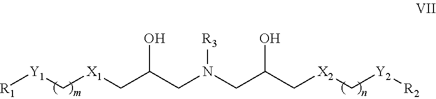

wherein:
Y₁ and Y₂ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, and alkynylene, wherein R at each occurrence is independently hydrogen, or an alkyl, alkenyl, alkynyl, carbocyclyl, heterocyclyl, group;
X₁ and X₂ are the same or different and are independently selected from —S—, —S—S—, —O—, —CH₂—, alkenylene, alkynylene, and —NR—, wherein R is hydrogen, or an alkyl, alkenyl, alkynyl, carbocyclyl, heterocyclyl, group;
R₁ and R₂ are the same or different and independently selected from alkyl, alkenyl, alkynyl, and heterocyclyl, each optionally substituted;
R₃ is selected from hydrogen, halogen, alkyl, alkenyl, alkynyl, carbocyclyl, heterocyclyl, aryl, and heteroaryl groups, each optionally substituted;
m and n are each independently an integer selected from 1 to 24,
wherein the longest chain of atoms in the compound is between 18 to 70 atoms.

In some embodiments, X₁, Y₁, and R₁ may together form part of a 4- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the carbocyclic or heterocyclic ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, X₂, Y₂, and R₂ may together form part of a 4- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the carbocyclic or heterocyclic ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, Y₁, X₁, X₂, and Y₂ may together form part of a 5- to 10-membered carbocyclic or heterocyclic ring having one or more heteroatoms selected from N, O, S, or P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the carbocyclic or heterocyclic ring may be optionally substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, X₁ and X₂ are each independently —S— or —O—.

In some embodiments, X₁ and X₂ are each independently oxygen (—O—), sulfur (—S—), or substituted or unsubstituted nitrogen (—NR—) or methylene.

In some embodiments, R₃ is selected from alkyl, cycloalkyl, or aryl, each optionally substituted by —OH.

In some embodiments, Y₁ and Y₂ are independently selected from —(C═O)O—, —O(C═O)—, —(C═O)S—, —S(C═O)—, —NR(C═O)—, and —(C═O)NR—.

In some embodiments, in the compounds of Formula VII:
Y₁ and Y₂ are the same or different, each independently selected from —(C═O)O—, —O(C═O)—, —NR(C═O)—, —(C═O)NR—, —O—, —CH₂—, an alkenylene, and an alkynylene, wherein R is independently a hydrogen, or an alkyl, alkenyl, alkynyl, carbocyclyl, heterocyclyl, group;
X₁ and X₂ are the same or different and are independently selected from —S—, —O—, —CH₂—, alkenylene, and alkynylene, —NR—, wherein R is hydrogen or an alkyl, alkenyl, alkynyl, carbocyclyl, heterocyclyl, group;
R₁ and R₂ are the same or different, each independently selected from carbocyclyl, alkyl, alkenyl, and alkynyl, each optionally substituted;
R₃ is selected from hydrogen, alkyl, alkenyl, alkynyl, carbocyclyl, heterocyclyl, aryl, and heteroaryl, each, except hydrogen, optionally substituted; and
m and n are each independently an integer selected from 1 to 14,
wherein the longest chain of atoms in the compound is between 18 and 70 atoms.

In some embodiments, the compound of Formula VII may be selected from the Compounds of List 7:

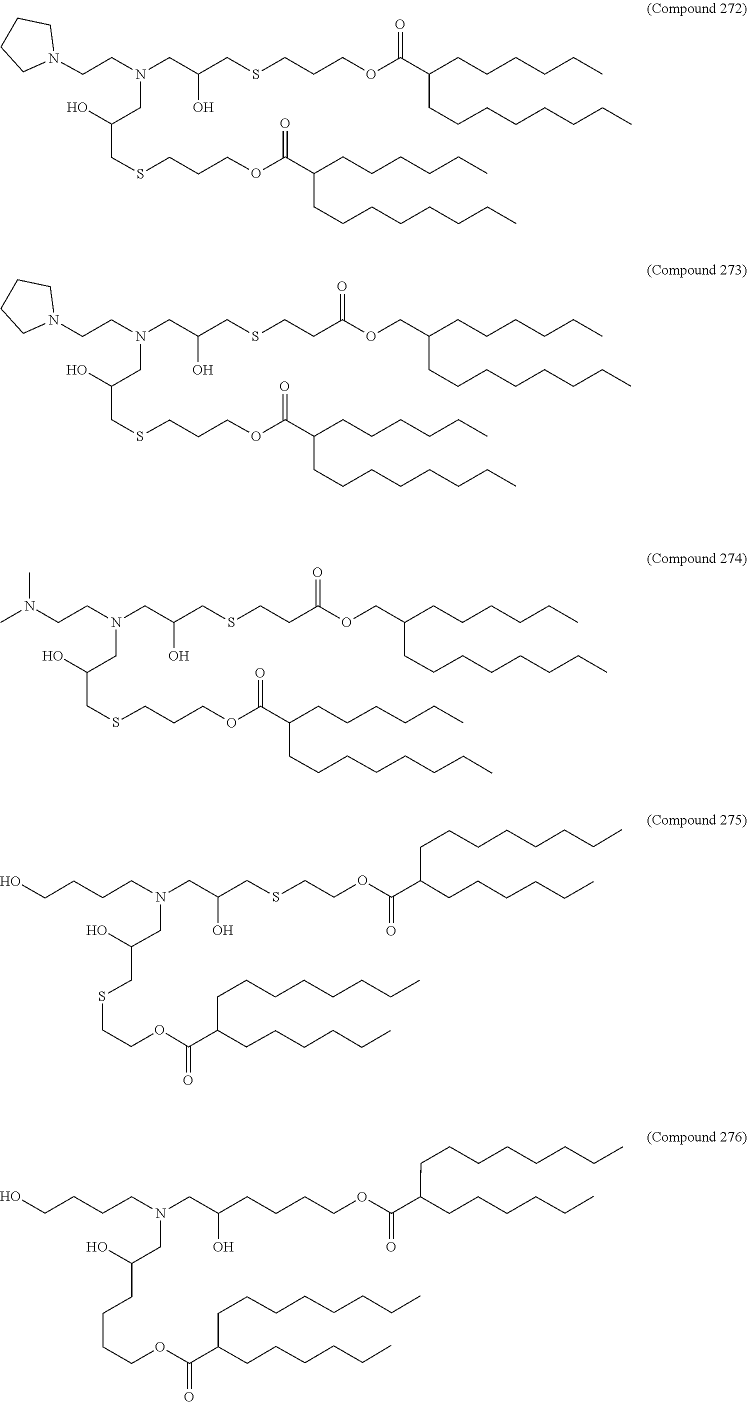

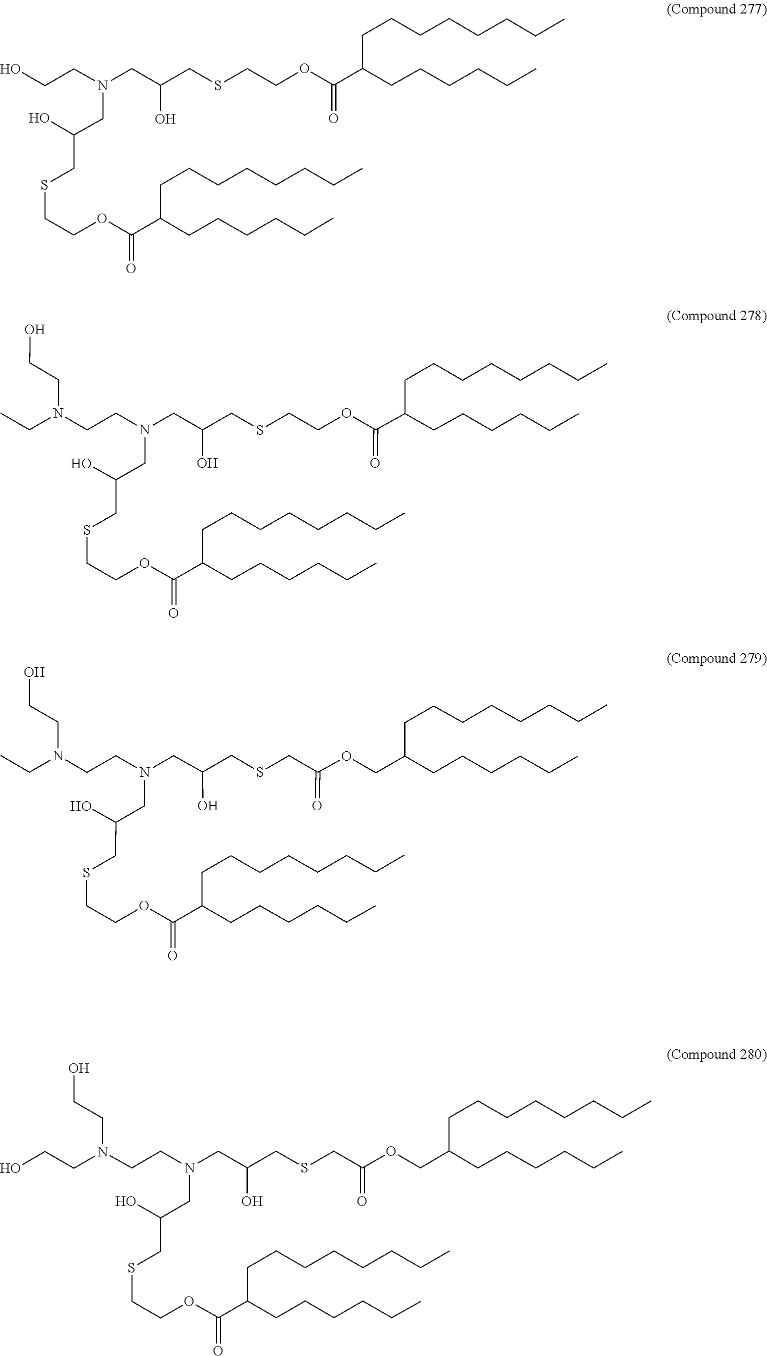

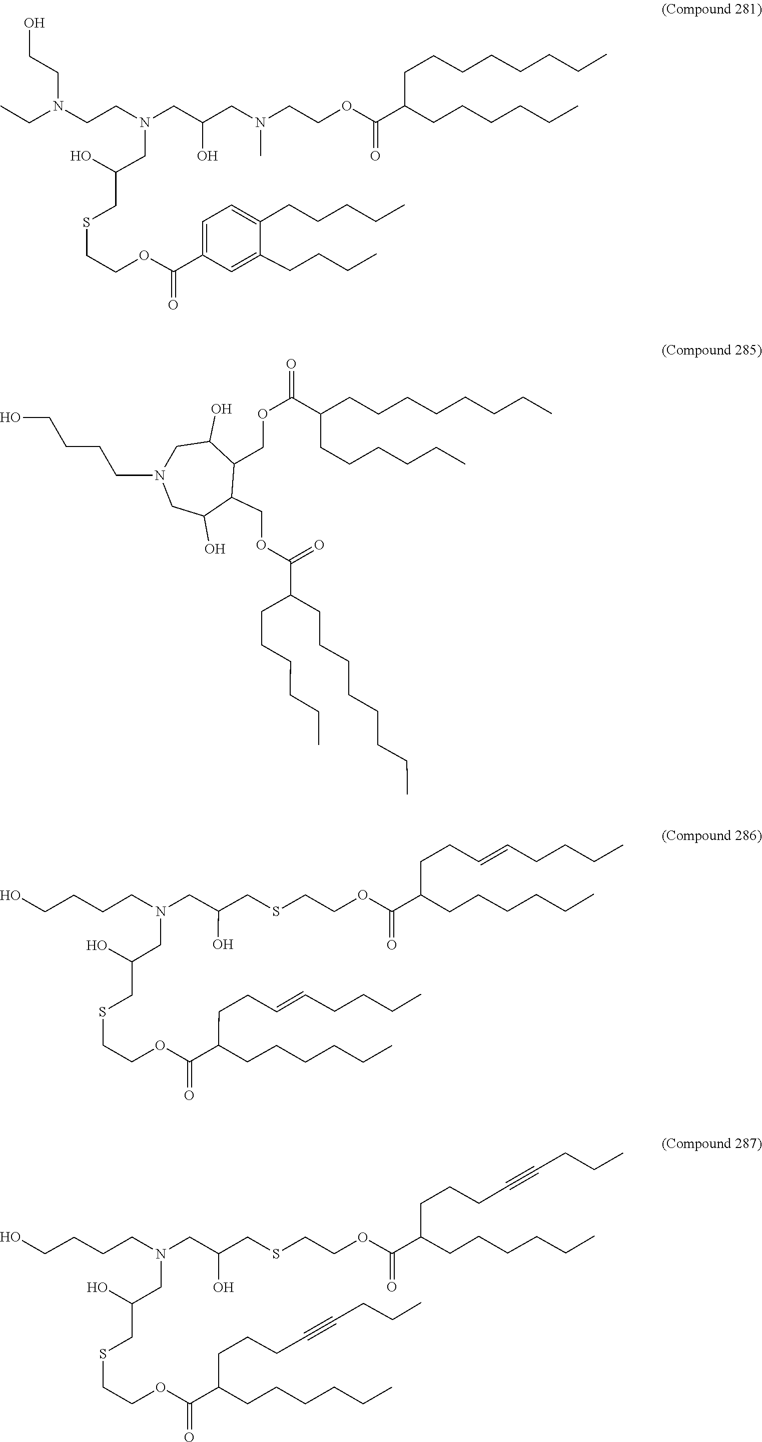 [SMILES: CCCCCCCCC(CCCCCC)C(=O)OCCN(C)CC(O)CN(CCN(CC)CCO)CC(O)CSCCOC(=O)c1ccc(CCCCC)c(CCCC)c1 ; CCCCCCCCC(CCCCCC)C(=O)OCC1C(O)CN(CCCCO)CC(O)C1COC(=O)C(CCCCCC)CCCCCCCC ; CCCC/C=C/CCC(CCCCCC)C(=O)OCCSCC(O)CN(CCCCO)CC(O)CSCCOC(=O)C(CC/C=C/CCCC)CCCCCC ; CCCC#CCCCC(CCCCCC)C(=O)OCCSCC(O)CN(CCCCO)CC(O)CSCCOC(=O)C(CCCC#CCCC)CCCCCC]

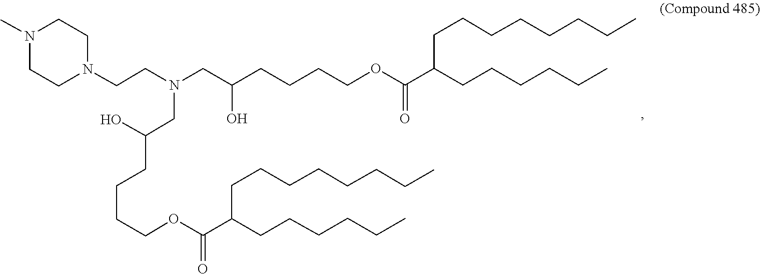 [SMILES: CCCCCCCCC(CCCCCC)C(=O)OCCCCC(O)CN(CCN1CCN(C)CC1)CC(O)CCCCOC(=O)C(CCCCCC)CCCCCCCC]

and salts or isomers thereof.

In another aspect of the disclosure, the compounds described herein are of Formula VIII or a salt or isomer thereof

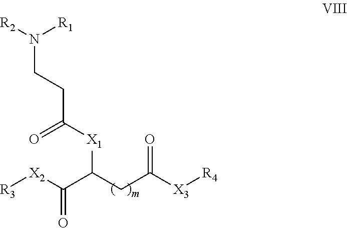

wherein:
X₁, X₂, and X₃ are independently selected from —CH₂—, —O—, —S—, and —NR—, wherein R is hydrogen or an alkyl, alkenyl, alkynyl, carbocyclyl, heterocyclyl, group;
R₁ and R₂ are the same or different and are independently selected from hydrogen, halogen, alkyl, alkenyl, alkynyl, carbocyclyl, heterocyclyl, aryl, and heteroaryl groups, each, except hydrogen and halogen, optionally substituted;
R₃ and R₄ are selected from carbocyclyl, alkyl, alkenyl, and alkynyl, each optionally substituted; and
m is 0, 1, 2, 3, 4, or 5,
wherein the longest chain of atoms in the compound is between 18 and 70 atoms.

In some embodiments, R₁ and R₂ may together form part of a 4- to 10-membered aromatic or non-aromatic heterocycle having one or more heteroatoms selected from N, O, S, and P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the heterocycle formed by R₁ and R₂ may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₁ and X may together form part of a 5- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the heterocycle formed by R₁ and X may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₃ and R₄ may together form part of a 5- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the heterocycle formed by R₃ and R₄ may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₁ and R₂ are independently selected from methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl, each optionally substituted by —OH, or together form a part of a ring structure.

In some embodiments, X₁, X₂ and X₃ are independently selected from oxygen (—O—), sulfur (—S—), and substituted or unsubstituted nitrogen (—NR—).

In some embodiments, in the compounds of Formula VIII:
X₁, X₂, and X₃ are independently selected from —CH₂—, —O—, and —NR—, wherein R is hydrogen or an alkyl, alkenyl, carbocyclyl, heterocyclyl, group;
R₁ and R₂ are the same or different and are independently selected from hydrogen, alkyl, alkenyl, alkynyl, carbocyclyl, and heterocyclyl, groups, each, except hydrogen, are optionally substituted;
R₃ and R₄ are selected from carbocyclyl, alkyl, alkenyl, and alkynyl, each optionally substituted;
m is 0, 1, 2, 3, 4, or 5.

In some embodiments, the compound of Formula VIII may be selected from the Compounds of List 8:

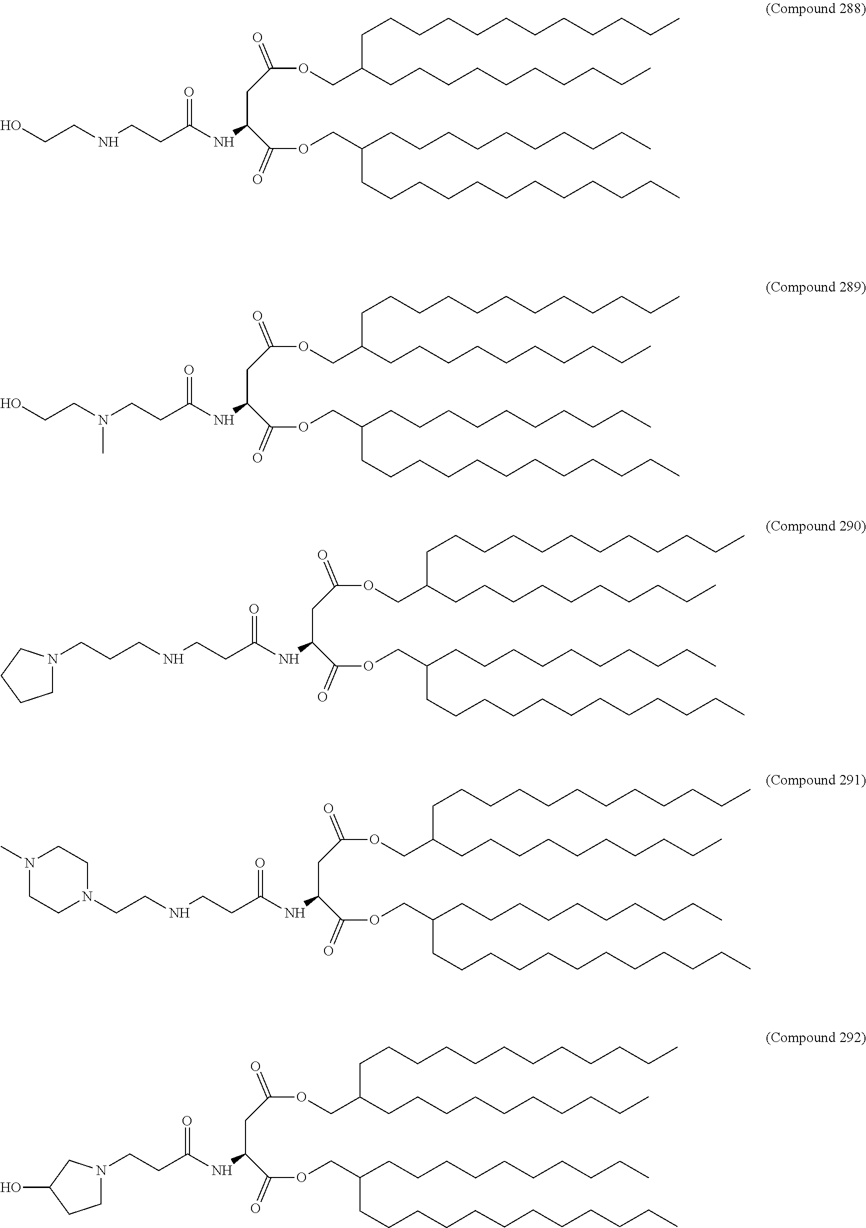

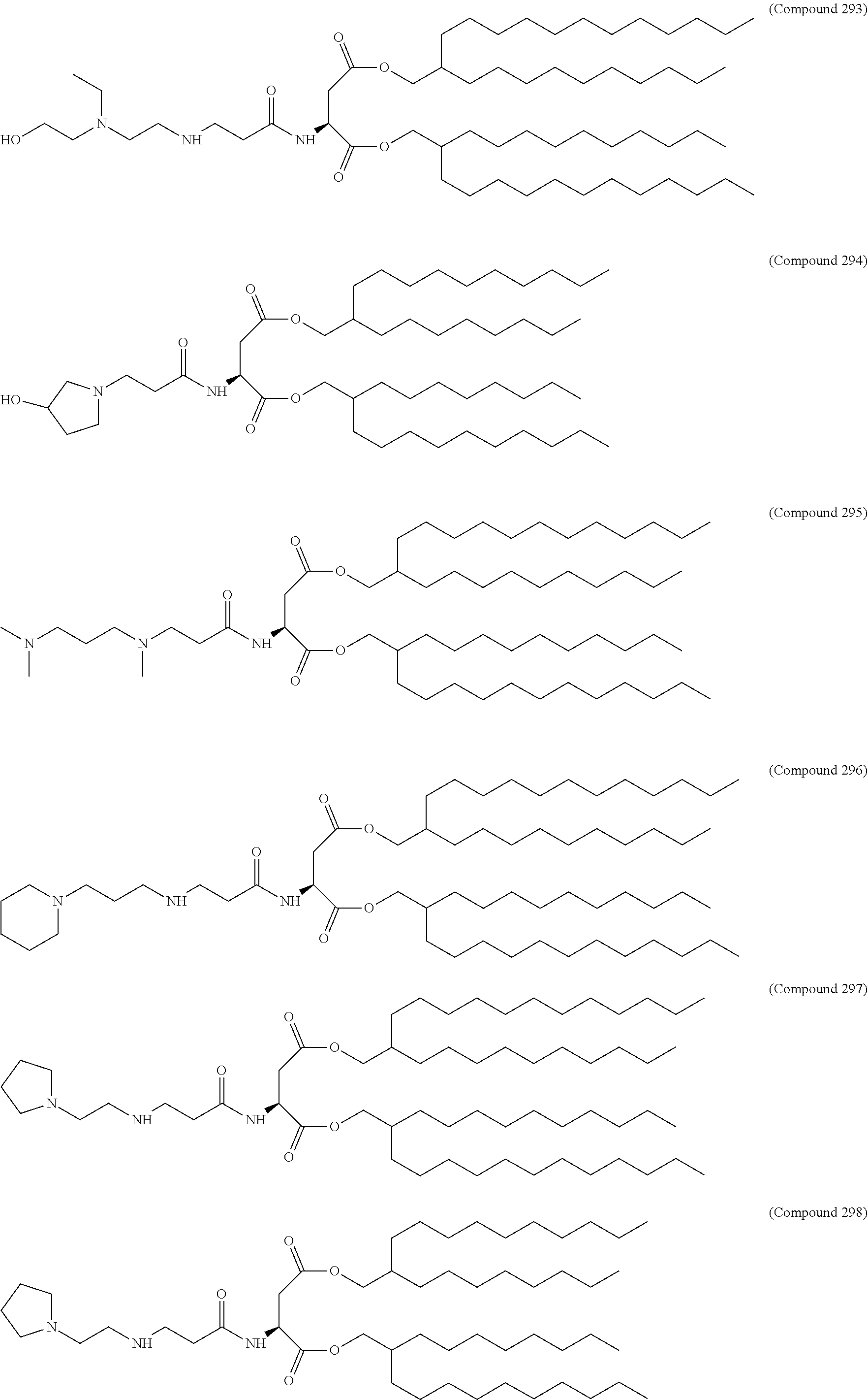

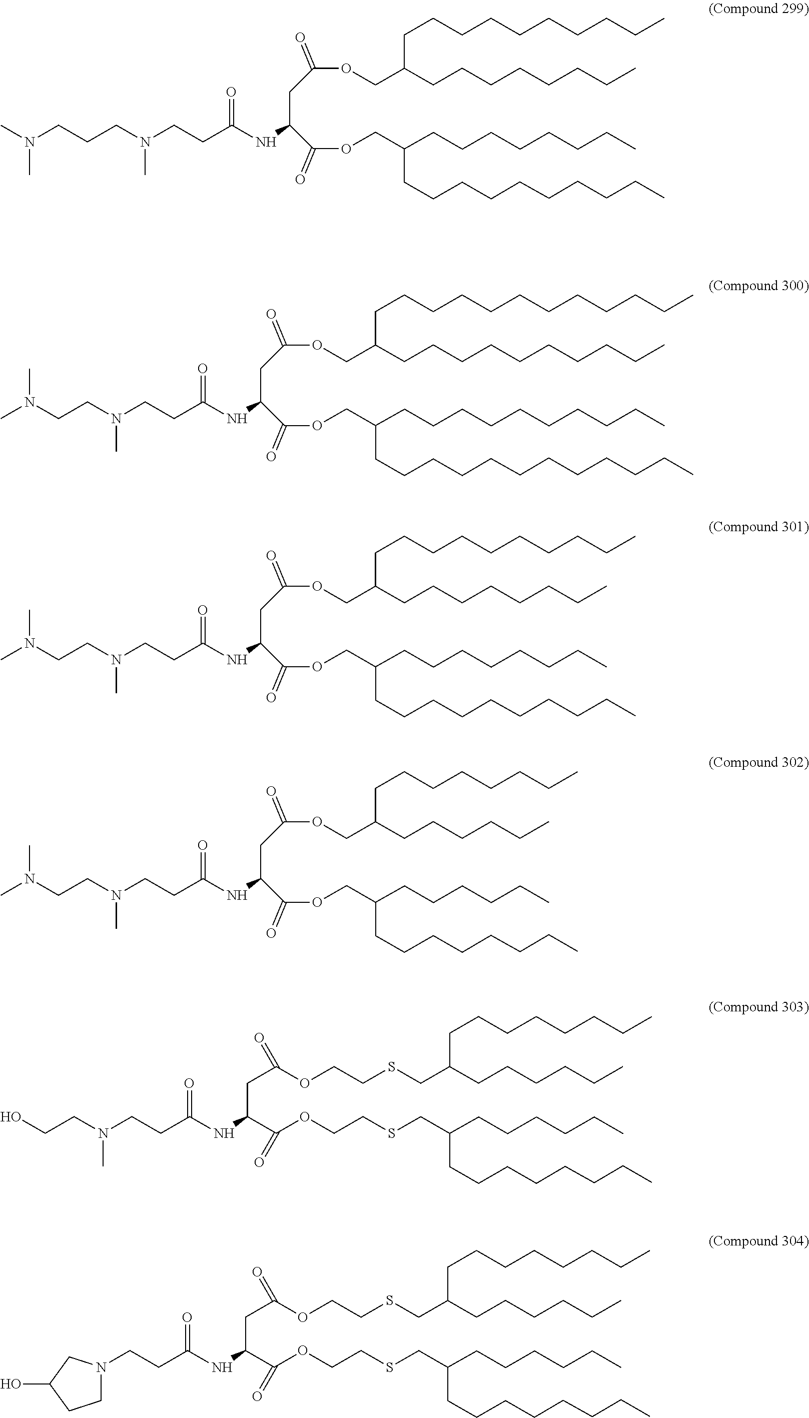

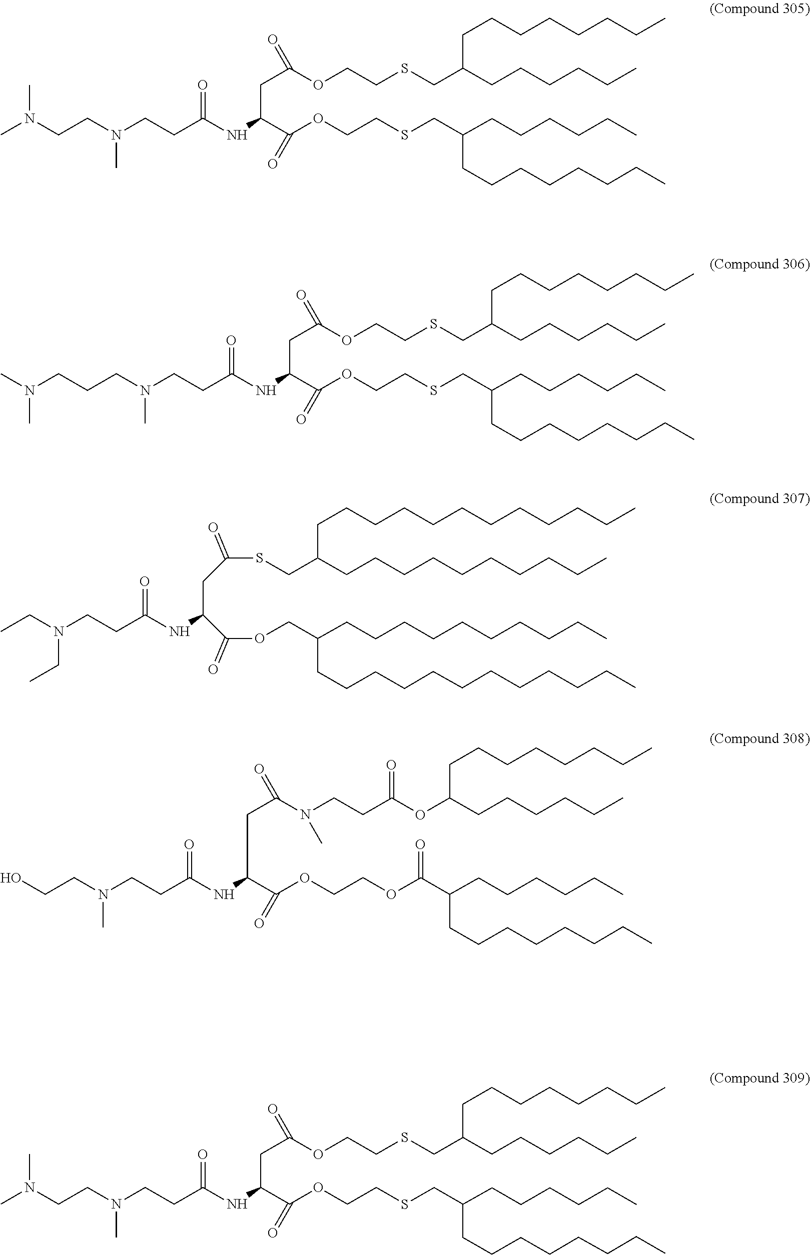

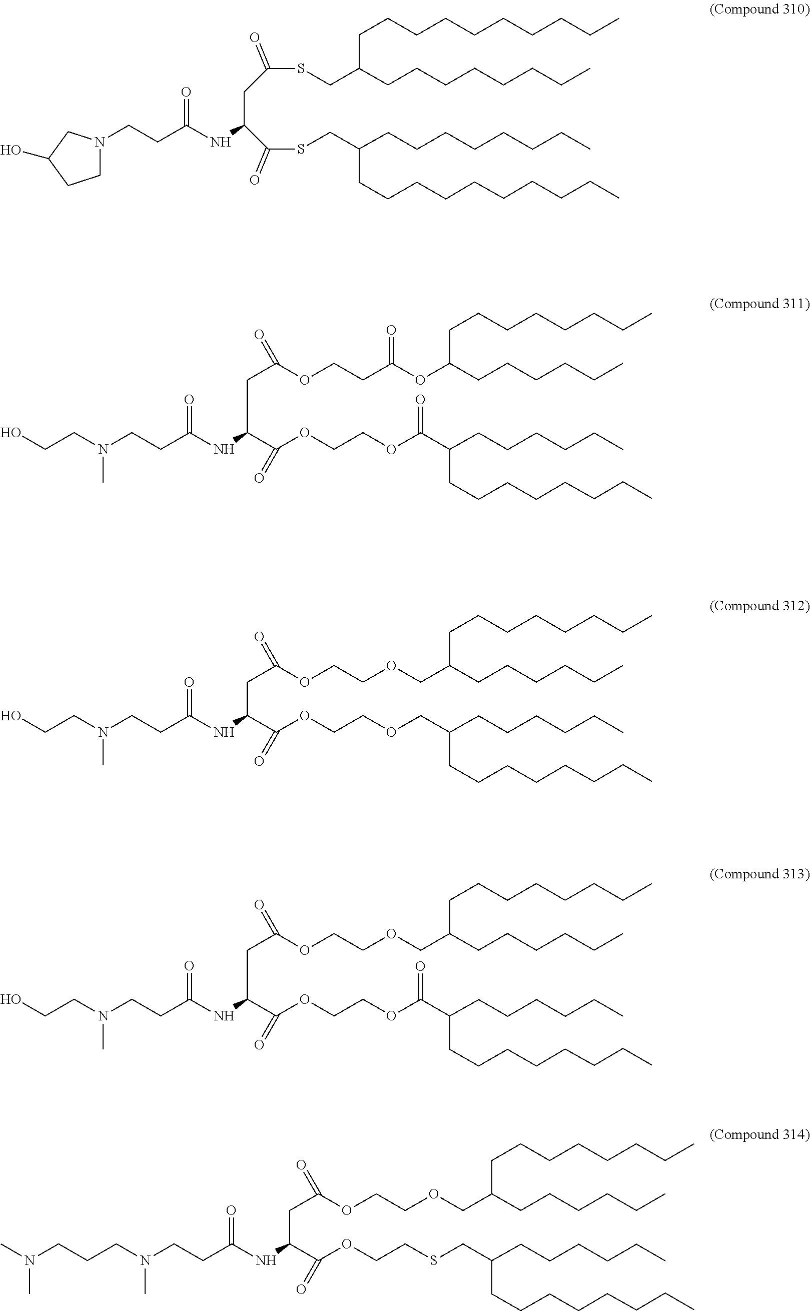

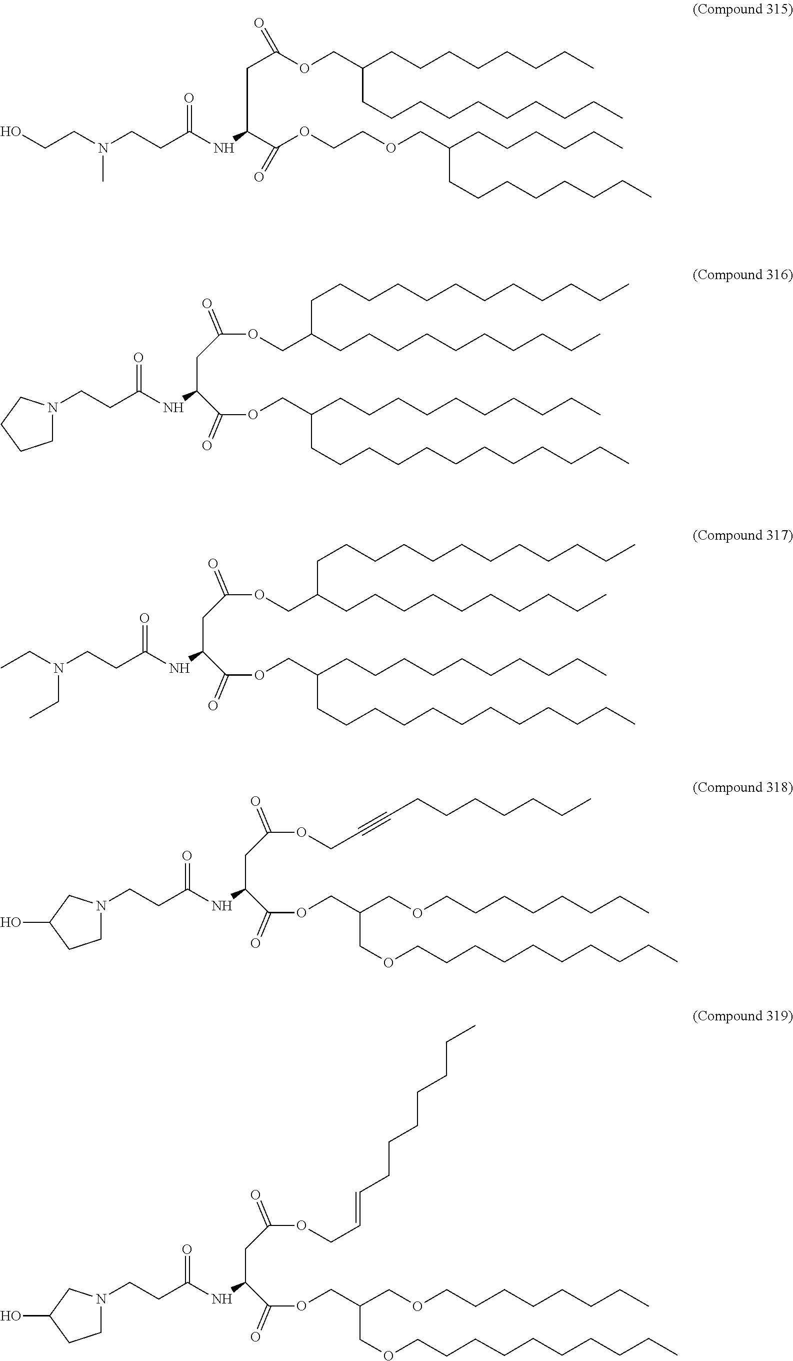

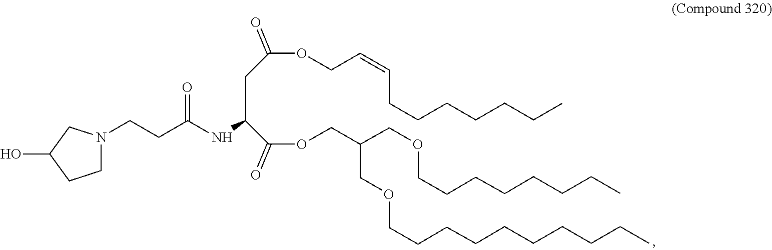 [SMILES: CCCCCCC/C=C\COC(=O)C[C@H](NC(=O)CCN1CCC(O)C1)C(=O)OCC(COCCCCCCCC)COCCCCCCCCCC]

or salts or isomers thereof.

In another aspect of the disclosure, the compounds described herein are of Formula IX or a salt or isomer thereof

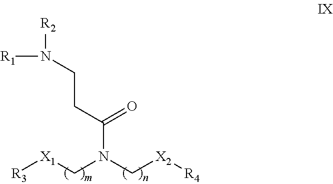

wherein:
X₁ and X₂ are the same or different moieties selected from —(C═O)O—, —O(C═O)—, —(C═O)S—, —S(C═O)—, —NR(C═O)—, —(C═O)NR—, —O—, —S—, —S—S—, —CH₂—, an alkene, and an alkyne, wherein R at each occurrence is independently hydrogen, or an alkyl, alkenyl, alkynyl, carbocyclyl, heterocyclyl group;
R₁ and R₂ are the same or different and are independently selected from hydrogen, halogen, alkyl, alkenyl, alkynyl, carbocyclyl, heterocyclyl, aryl, and heteroaryl groups, each except hydrogen and halogen optionally substituted;
R₃ and R₄ are the same or different and are independently selected from carbocyclyl, alkyl, alkenyl, and alkynyl; and
m and n are the same or different and are each an integer selected from 1 to 24,
wherein the longest chain of atoms in the compound is between 18 and 70 atoms.

In some embodiments, R₁ and R₂ may together form part of a 4- to 10-membered aromatic or non-aromatic heterocycle having one or more heteroatoms selected from N, O, S, and P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, X₁ and X₂ may together form part of a 4- to 10-membered aromatic or non-aromatic heterocycle having one or more heteroatoms selected from N, O, S, and P, which can optionally be part of a functional group such as, but not limited to, ethers, sulfides, disulfides, esters, sulfonate esters, thioesters, sulfones, sulfoxides, amines, amides, carbamates, carbonates, alkenes, or alkynes. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, wherein R₁ and R₂ are each independently a C₁₋₂₀ alkyl, each optionally substituted by —OH.

In some embodiments, X₁ and X₂ are independently selected from oxygen (—O—), sulfur (—S—), substituted or unsubstituted nitrogens (—NR—), esters, thioesters, amides, alkenes, and alkynes.

In some embodiments, in the compounds of Formula IX:
X₁ and X₂ are the same or different moieties selected from —(C═O)O—, —O(C═O)—, —NR(C═O)—, —(C═O)NR—, —O—, —CH₂—, an alkene, and an alkyne, wherein R at each occurrence is independently hydrogen, or an alkyl, alkenyl, alkynyl, carbocyclyl, heterocyclyl group;
R₁ and R₂ are the same or different and are independently selected from hydrogen, alkyl, alkenyl, alkynyl, carbocyclyl, and heterocyclyl groups, each except hydrogen and halogen optionally substituted;
R₃ and R₄ are the same or different and are independently selected from carbocyclyl, alkyl, alkenyl, and alkynyl; and
m and n are the same or different and are each an integer selected from 1 to 14,
wherein the longest chain of atoms in the compound is between 18 to 70 atoms.

In some embodiments, the compound of Formula IX may be selected from the Compounds of List 9:

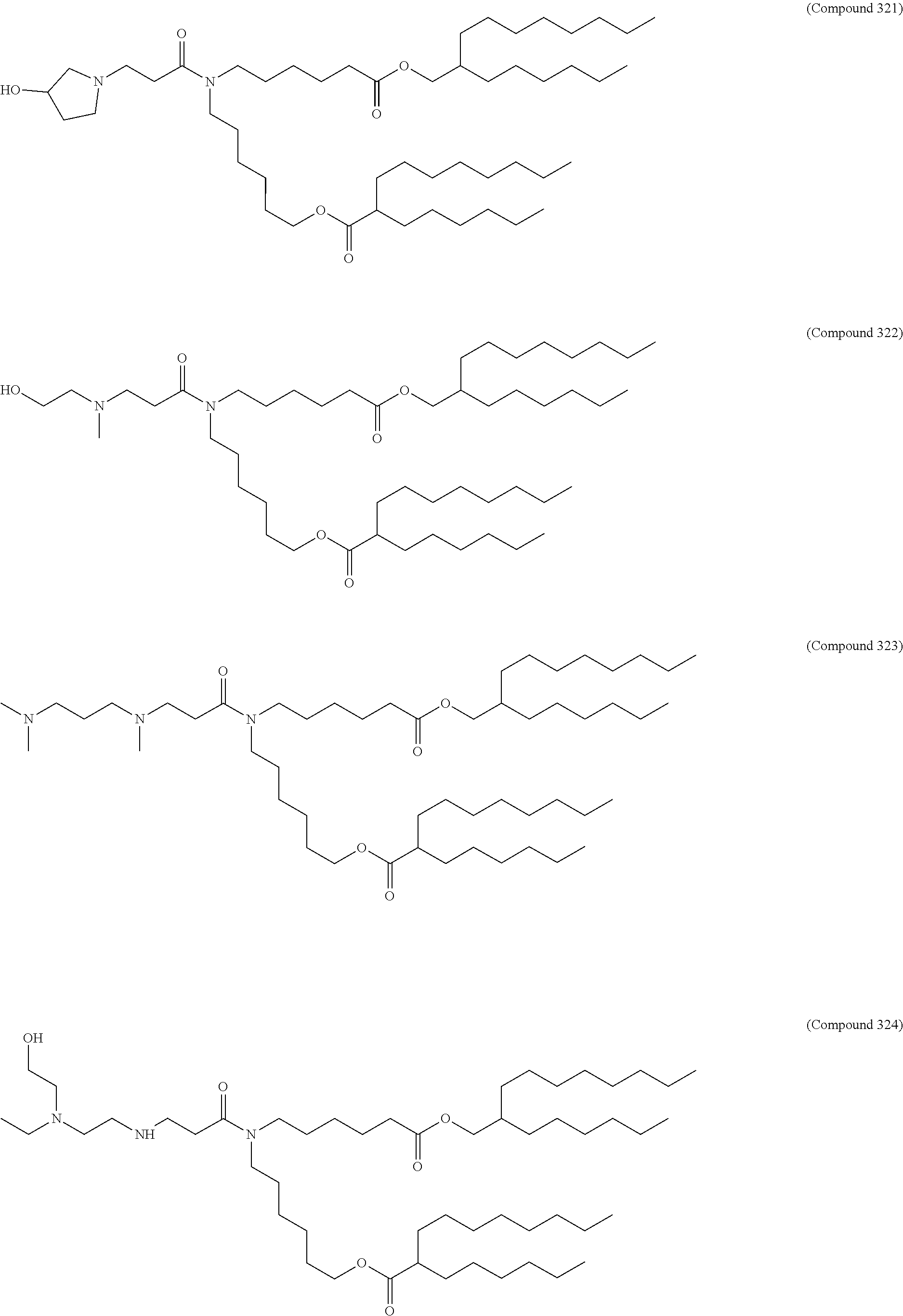

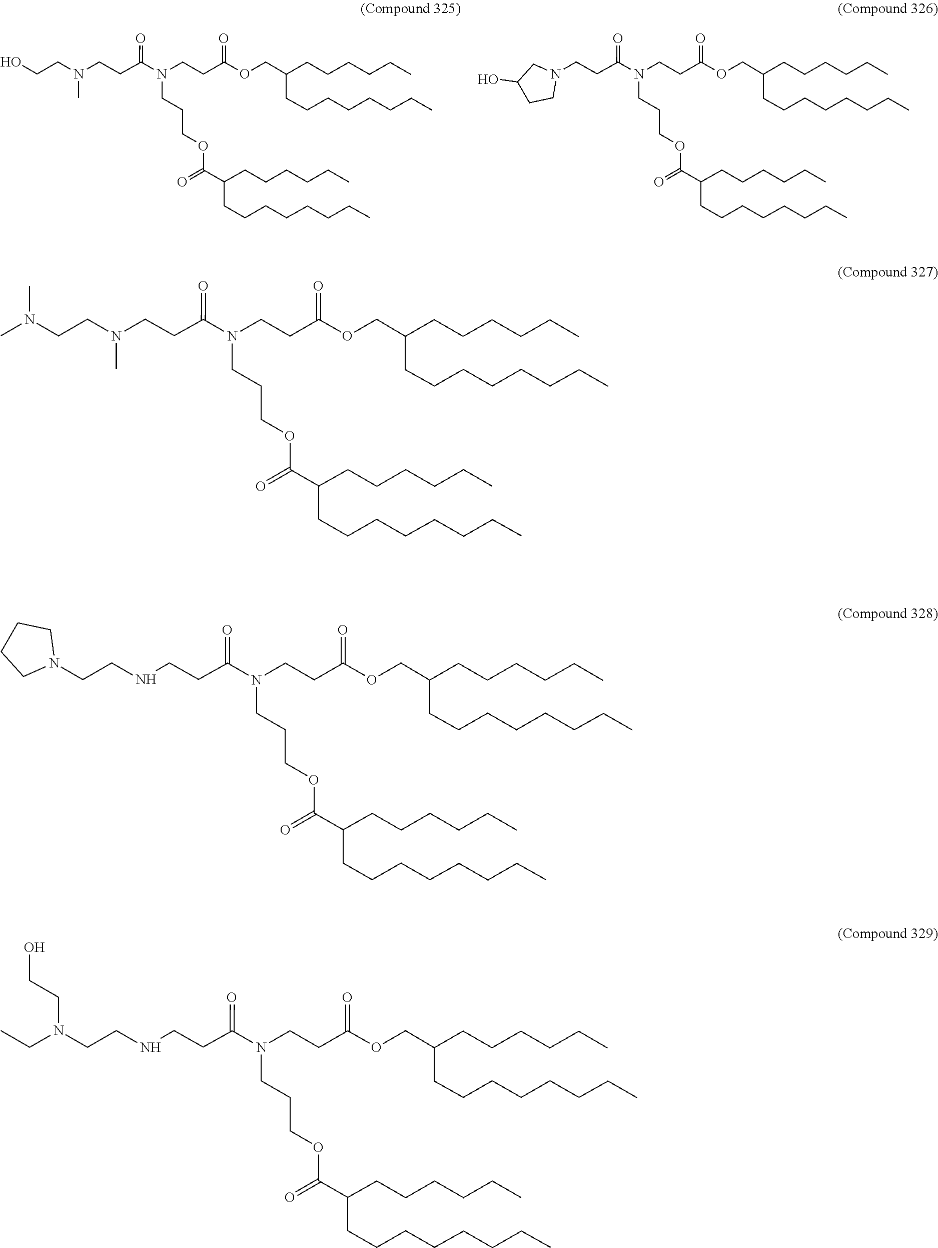

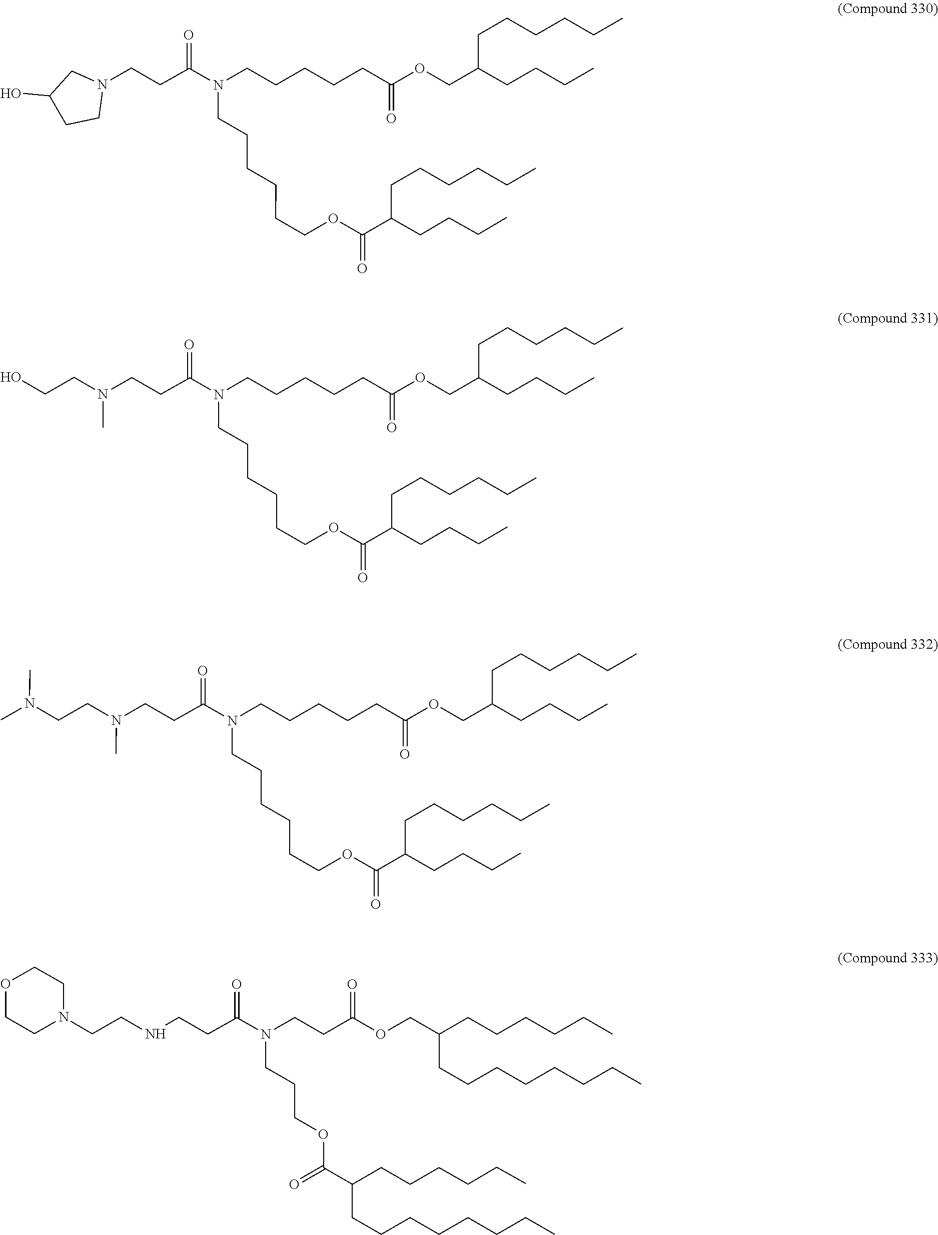

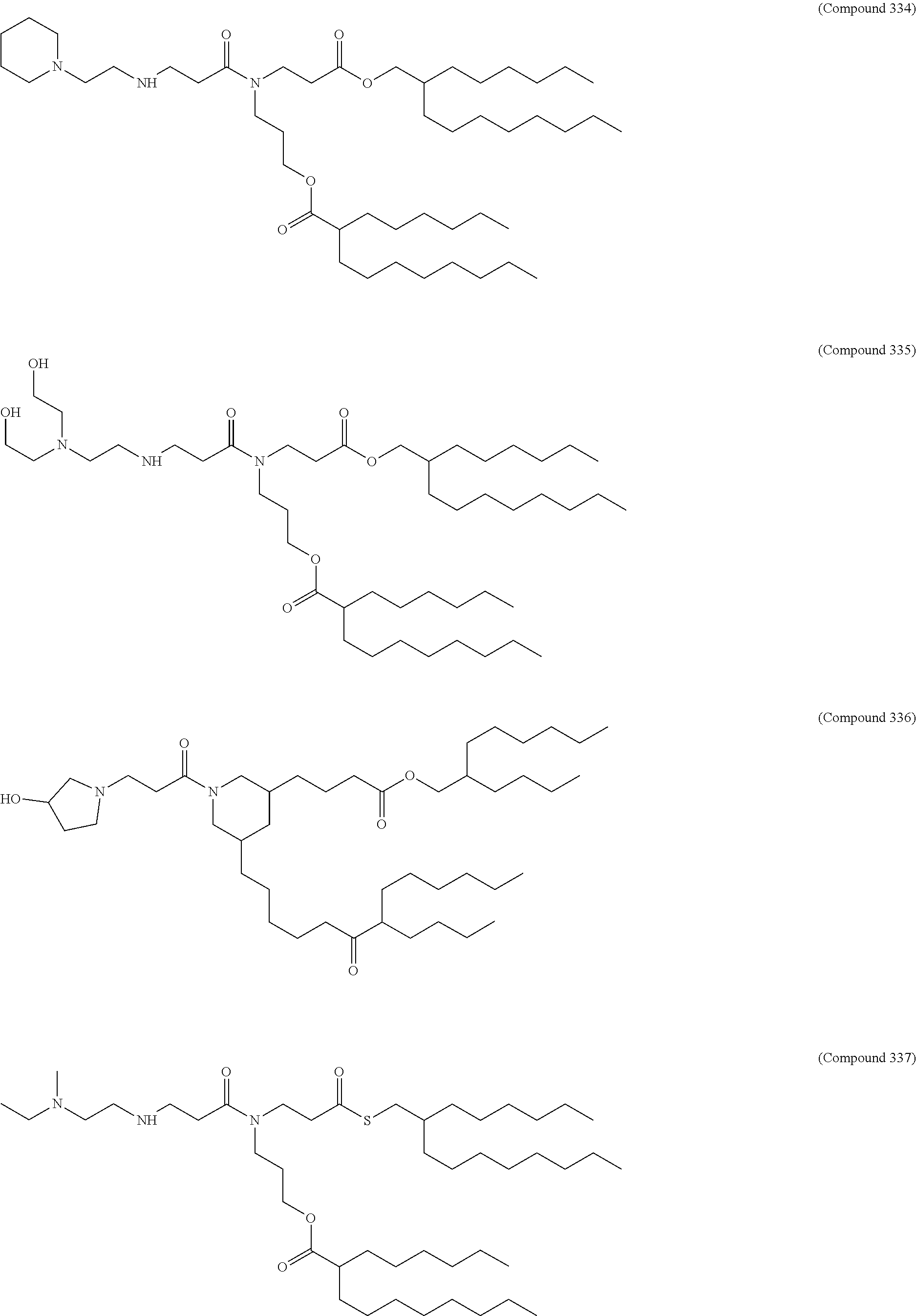

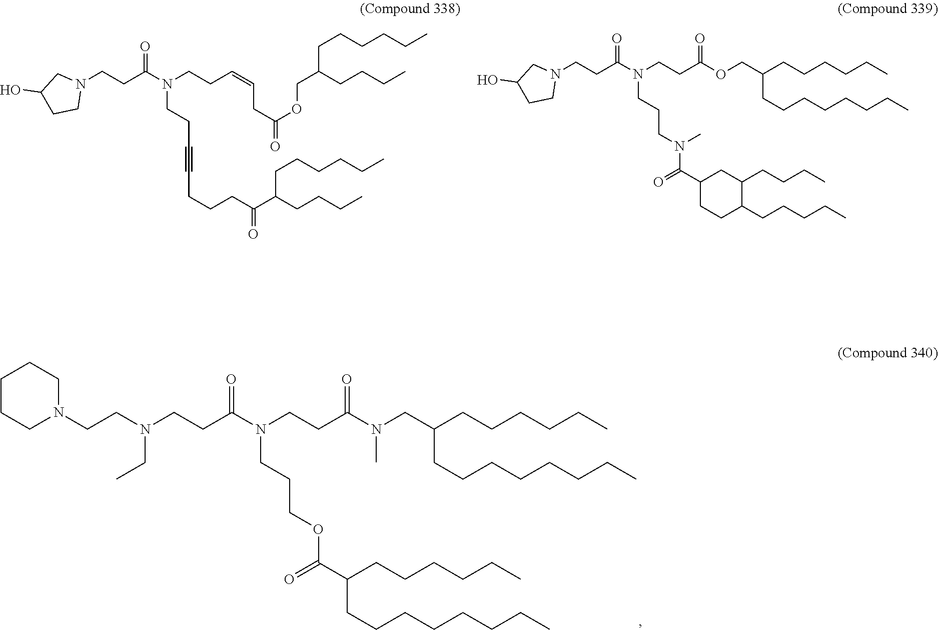 [SMILES: CCCCCCC(CCCC)COC(=O)C/C=C\CCN(CCC#CCCCC(=O)C(CCCC)CCCCCC)C(=O)CCN1CCC(O)C1 ; CCCCCCCCC(CCCCCC)COC(=O)CCN(CCCN(C)C(=O)C1CCC(CCCCC)C(CCCC)C1)C(=O)CCN1CCC(O)C1 ; CCCCCCCCC(CCCCCC)CN(C)C(=O)CCN(CCCOC(=O)C(CCCCCC)CCCCCCCC)C(=O)CCN(CC)CCN1CCCCC1]

or salts or isomers thereof.

In another aspect of the disclosure, the compounds described herein are of Formula X or a salt or isomer thereof

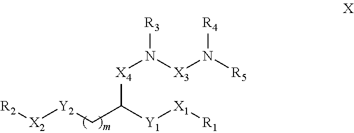

wherein:
R₁, R₂, R₃, R₄ and R₅ are the same or different and are independently selected from hydrogen, halogen, alkyl, alkenyl, alkynyl, carbocyclyl, heterocyclyl, aryl, and heteroaryl groups, each, except hydrogen and halogen, optionally substituted;
X₁, X₂, and X₃ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, and alkynylene, wherein R at each occurrence is independently hydrogen, or an alkyl, alkenyl, alkynyl, carbocyclyl, heterocyclyl, group;
X₄ is selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, and alkynylene, wherein R at each occurrence is independently hydrogen, or an alkyl, alkenyl, alkynyl, carbocyclyl, heterocyclyl, group; or X₄ is a linear alkyl group comprising 0 to 10 methylene units;
Y₁ and Y₂ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, or alkynylene, and carbocyclylene, wherein R at each occurrence is independently hydrogen, alkyl, alkenyl, alkynyl, carbocyclyl, or heterocyclyl; and
m is 0, 1, 2, 3, 4, 5, 6, 7, 8, 9, or 10,
wherein the longest chain of atoms in the compound is between 18 and 70 atoms.

In some embodiments, R₃, R₄, and X₃ may together with the atoms they are attached, form part of a 4- to 10-membered aromatic or non-aromatic heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₄ and R₅ may together with the atoms they are attached, form part of a 4- to 10-membered aromatic or non-aromatic heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, Y₁, X₁, and R₁ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, Y₂, X₂, and R₂ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₃ is methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl, each optionally substituted by —OH, or alternatively R₃ is fused into a ring system with R₄.

In some embodiments, R₄ and R₅ are independently selected from linear, branched, or cyclic alkyl groups, selected from methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, and icosyl, each optionally substituted by —OH, or alternatively R₄ and R₅ are fused into a ring system.

In some embodiments, X₁ and X₂ are independently selected from —(C═O)O—, —O(C═O)—, —NR(C═O)—, —(C═O)NR—, oxygen atoms, sulfur atoms, —NR—, alkenes, alkynes, or a cyclic alkyl or heteroalkyl and X₃ is selected from —O—, —S—, alkylene, amine, alkenylene, and alkynylene, wherein R at each occurrence is independently hydrogen, or an alkyl, alkenyl, alkynyl, carbocyclyl, heterocyclyl group.

In some embodiments, X₄ is —O—, —S—.

In some embodiments, Y₁ and Y₂ are independently selected from —(C═O)O—, —O(C═O)—, —NR(C═O)—, —(C═O)NR—, —O—, —S—, —NR—, alkenes, alkynes, or cycloalkylene or heteroalkylene, wherein R at each occurrence is independently hydrogen, or an alkyl, alkenyl, alkynyl, carbocyclyl, heterocyclyl group.

In some embodiments, in the compounds of Formula X:
R₁, R₂, R₃, R₄ and R₅ are the same or different and are independently selected from hydrogen, alkyl, alkenyl, alkynyl, carbocyclyl, heterocyclyl, aryl, and heteroaryl groups, each, except hydrogen, optionally substituted;
X₁, X₂, and X₃ are the same or different and are independently selected from —(C═O)O—, —O(C═O)—, —NR(C═O)—, —(C═O)NR—, —O—, —S—, —CH₂—, —NR—, alkenylene, and alkynylene, wherein R at each occurrence is independently hydrogen, or an alkyl, alkenyl, alkynyl, carbocyclyl, heterocyclyl, group;
X₄ is selected from —(C═O)O—, —O(C═O)—, —NR(C═O)—, —(C═O)NR—, —O—, —S—, —CH₂—, —NR—, alkenylene, and alkynylene, wherein R at each occurrence is independently hydrogen, or an alkyl, alkenyl, alkynyl, carbocyclyl, heterocyclyl, group; or X₄ is a linear alkyl group comprising 0 to 8 methylene units;
Y₁ and Y₂ are the same or different and are independently selected from —(C═O)O—, —O(C═O)—, —NR(C═O)—, —(C═O)NR—, —O—, —S—, —CH₂—, —NR—, alkylene, alkenylene, or alkynylene, and carbocyclylene, wherein R at each occurrence is independently hydrogen, alkyl, alkenyl, alkynyl, carbocyclyl, or heterocyclyl; and
m is 0, 1, 2, 3, 4, or 5,
wherein the longest chain of atoms in the compound is between 18 to 70 atoms.

In some embodiments, the compound of Formula X may be selected from the Compounds of List 10:

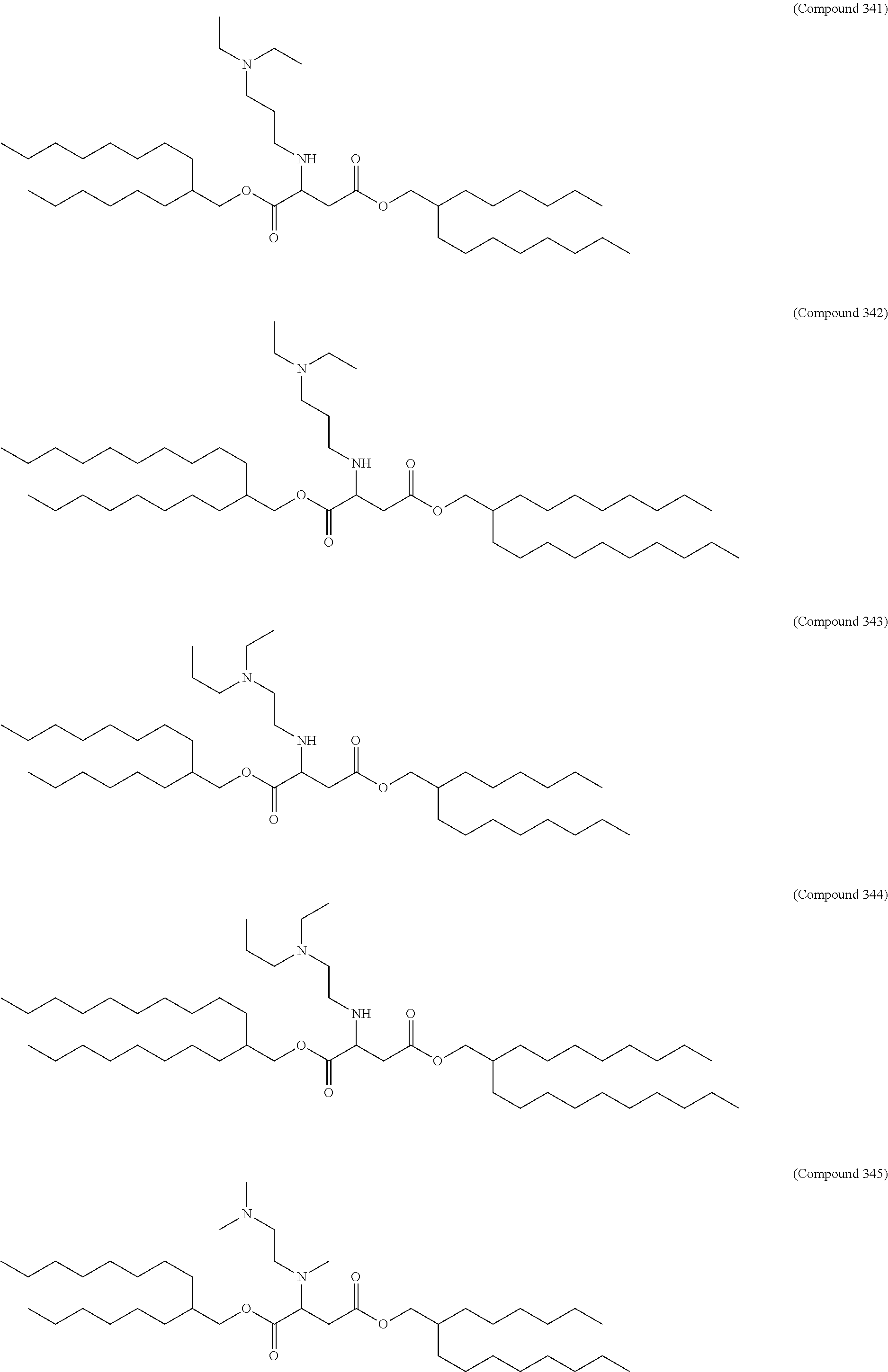

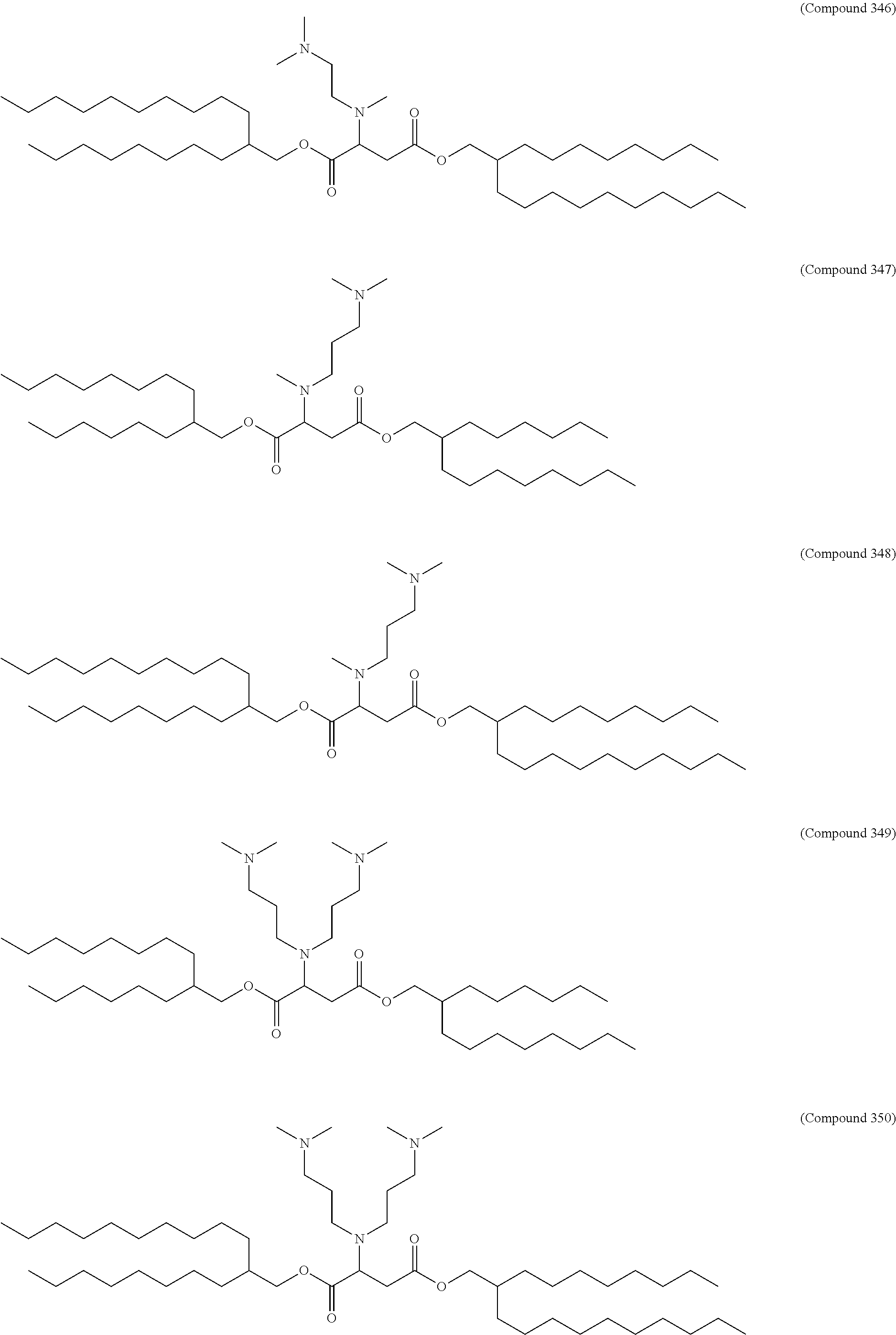

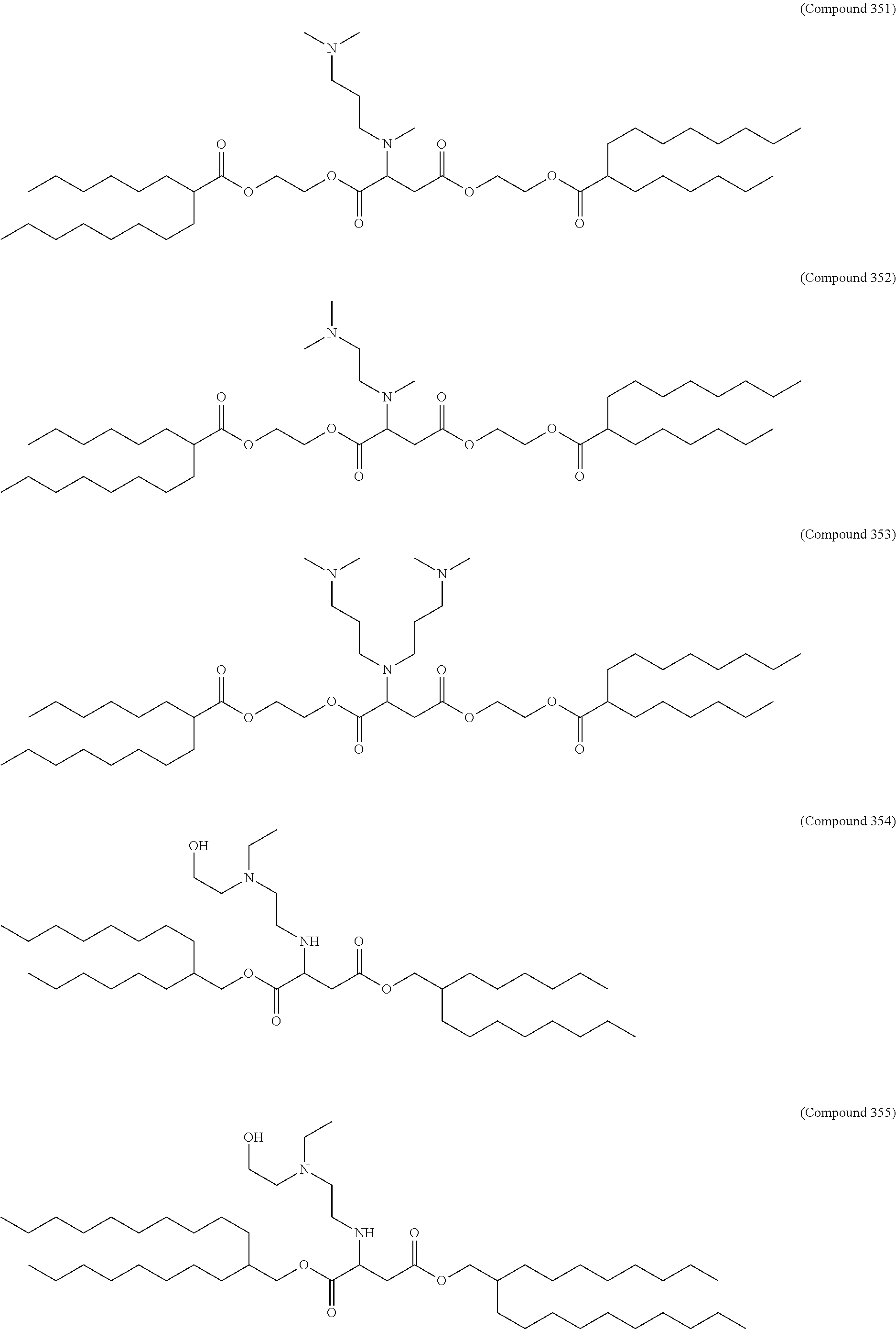

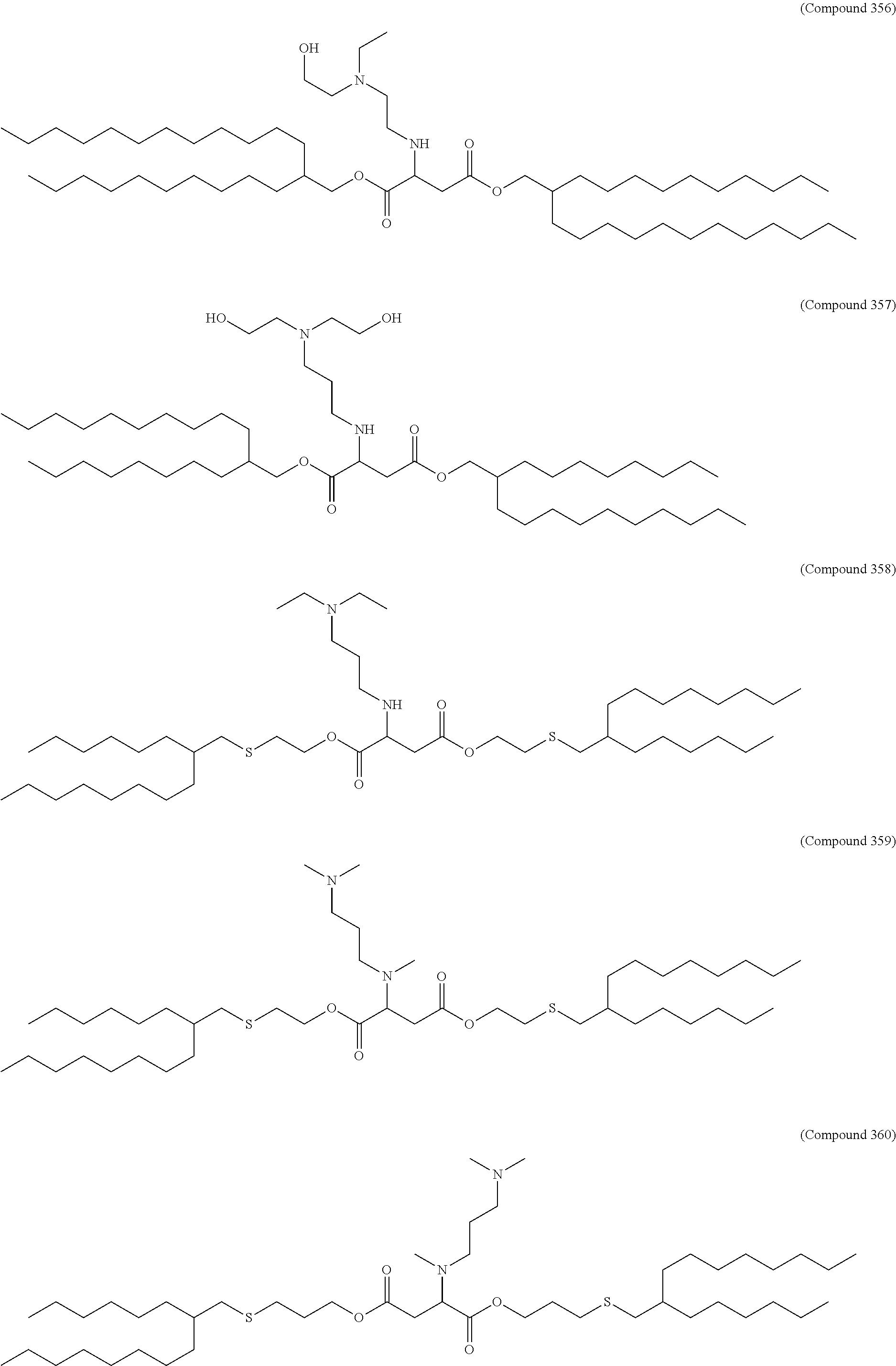

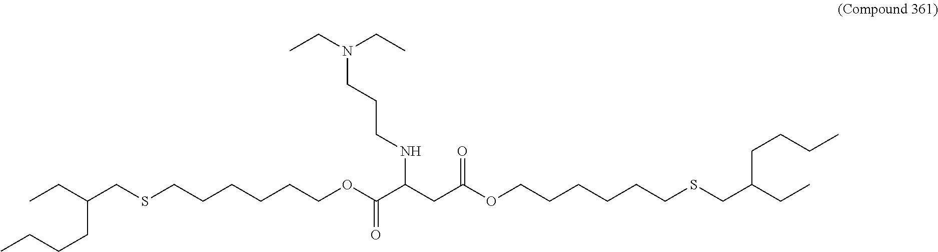 [SMILES: CCCCC(CC)CSCCCCCCOC(=O)CC(NCCCN(CC)CC)C(=O)OCCCCCCSCC(CC)CCCC]

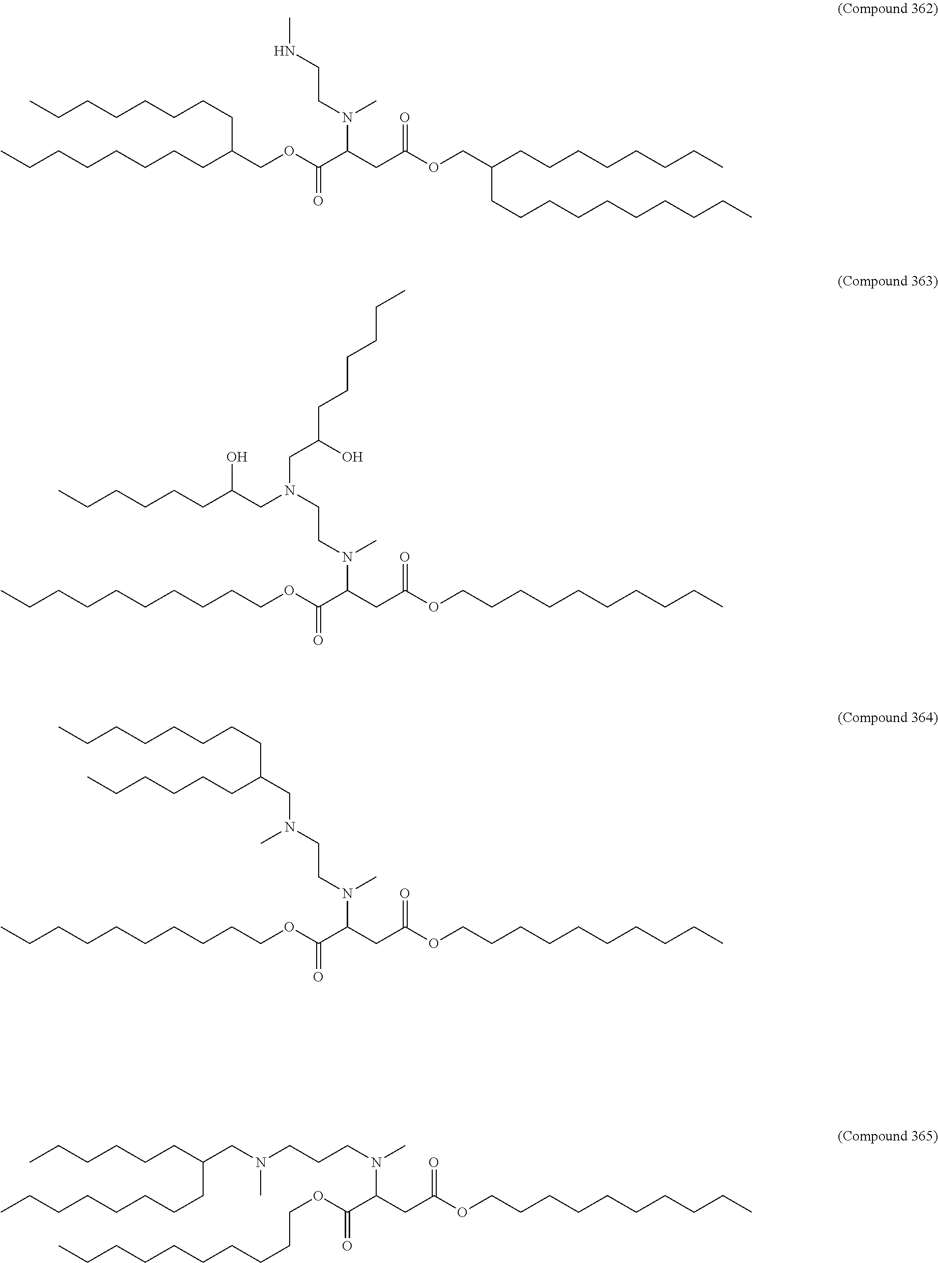

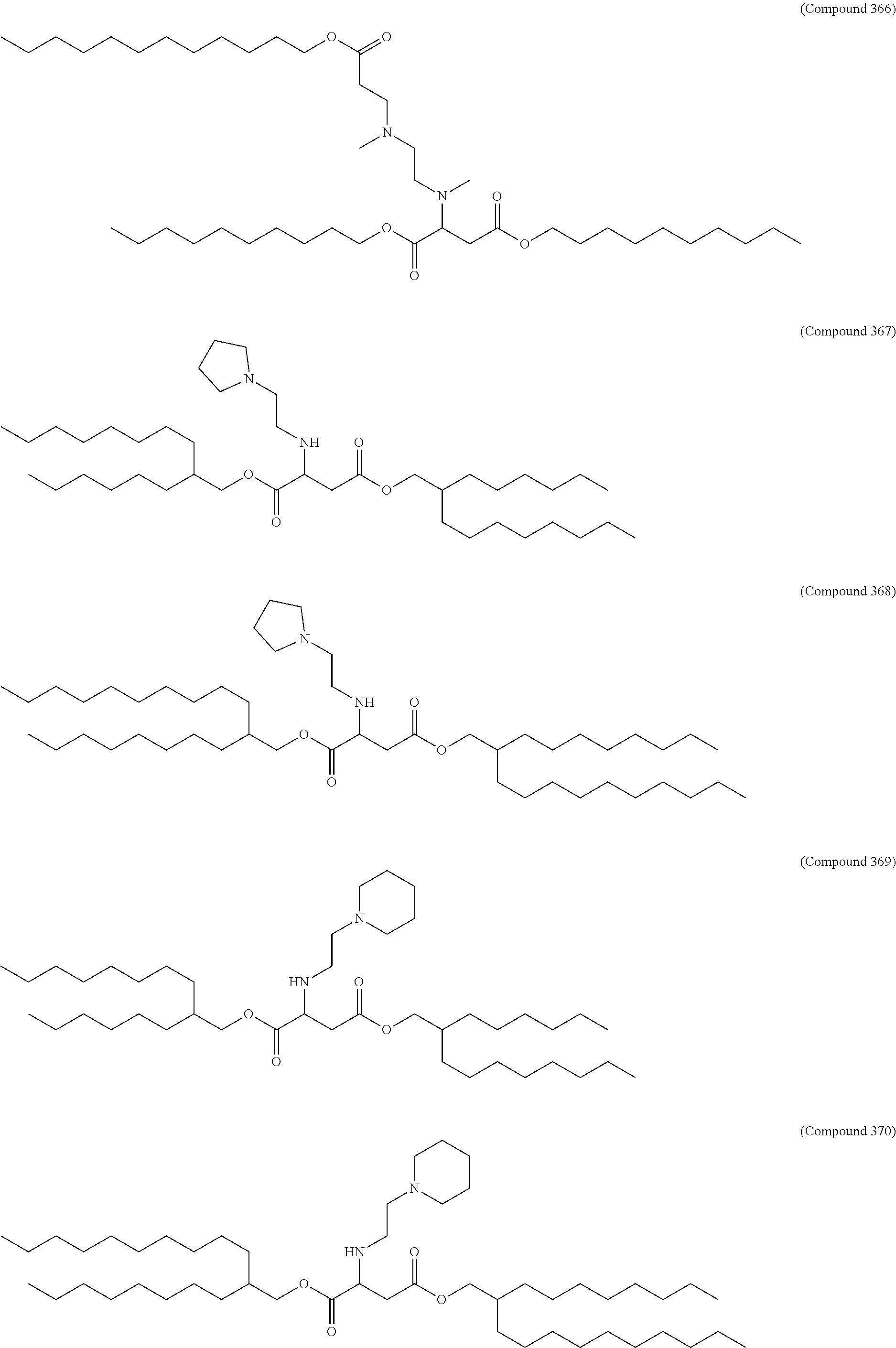

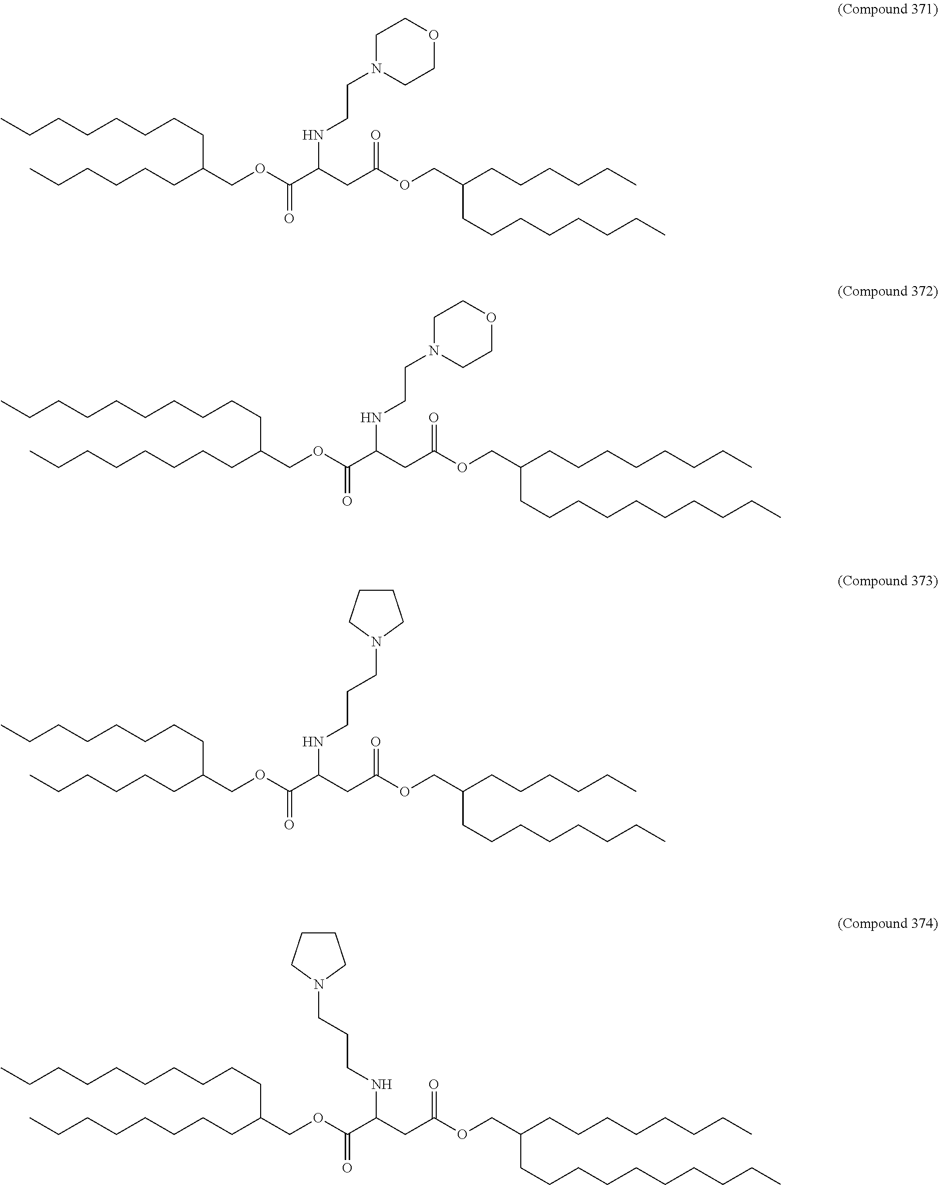

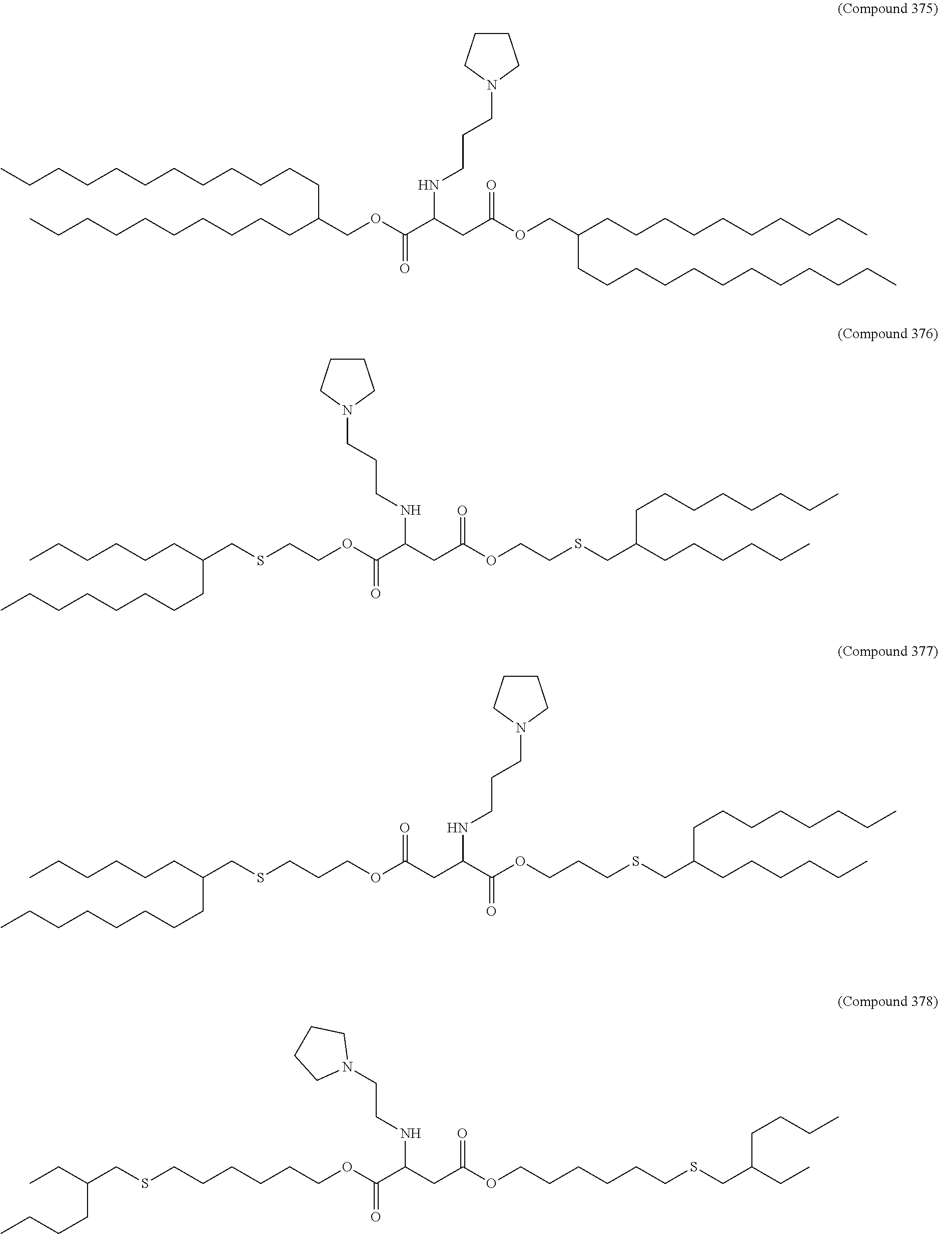

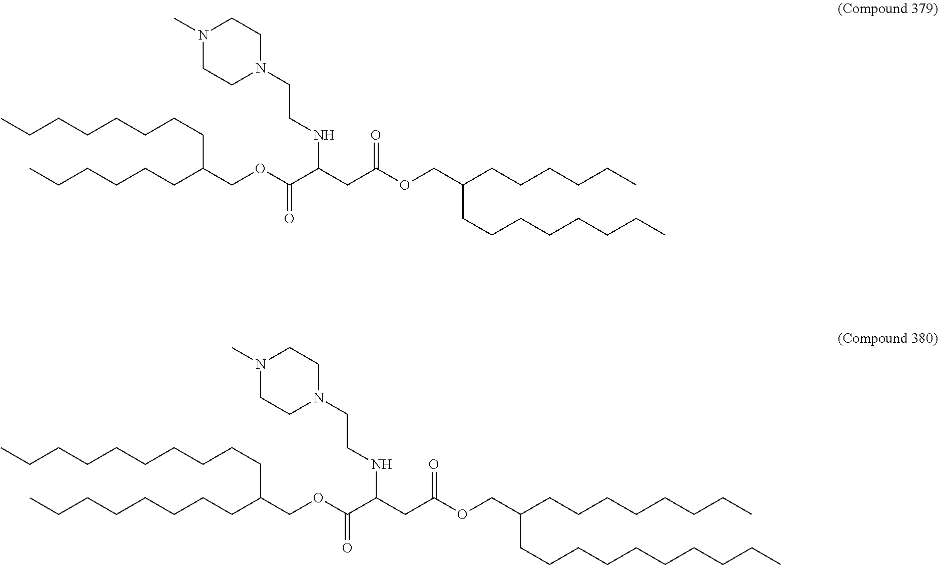

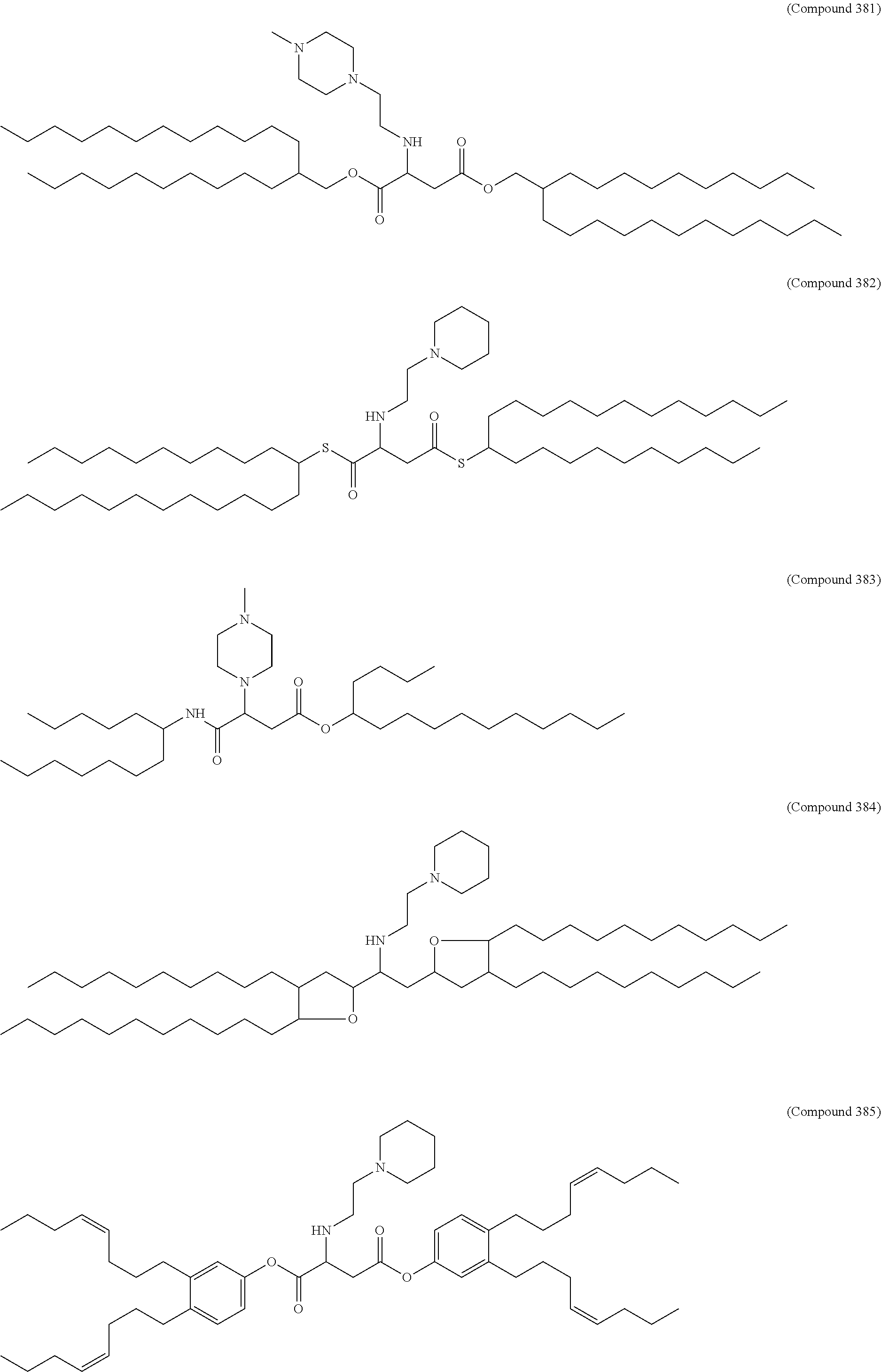

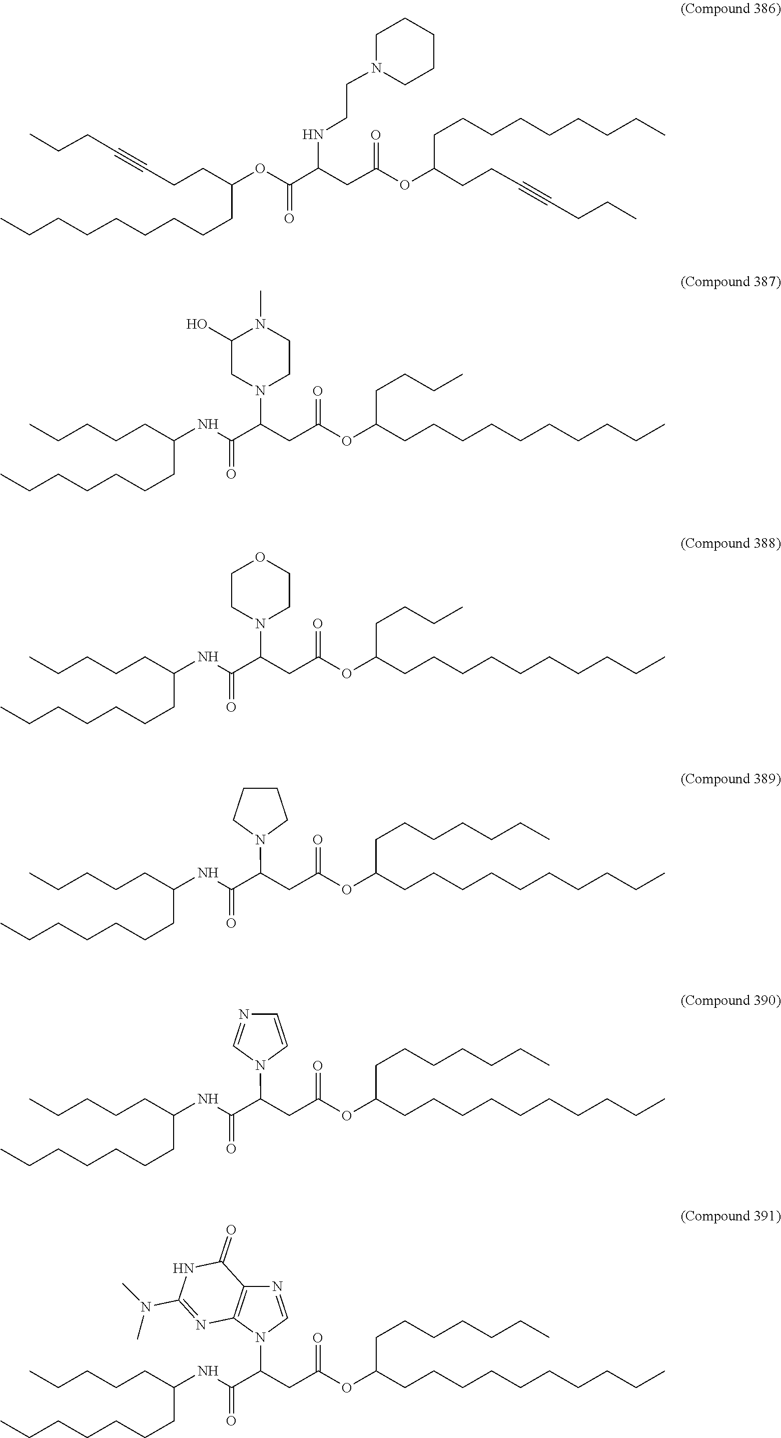

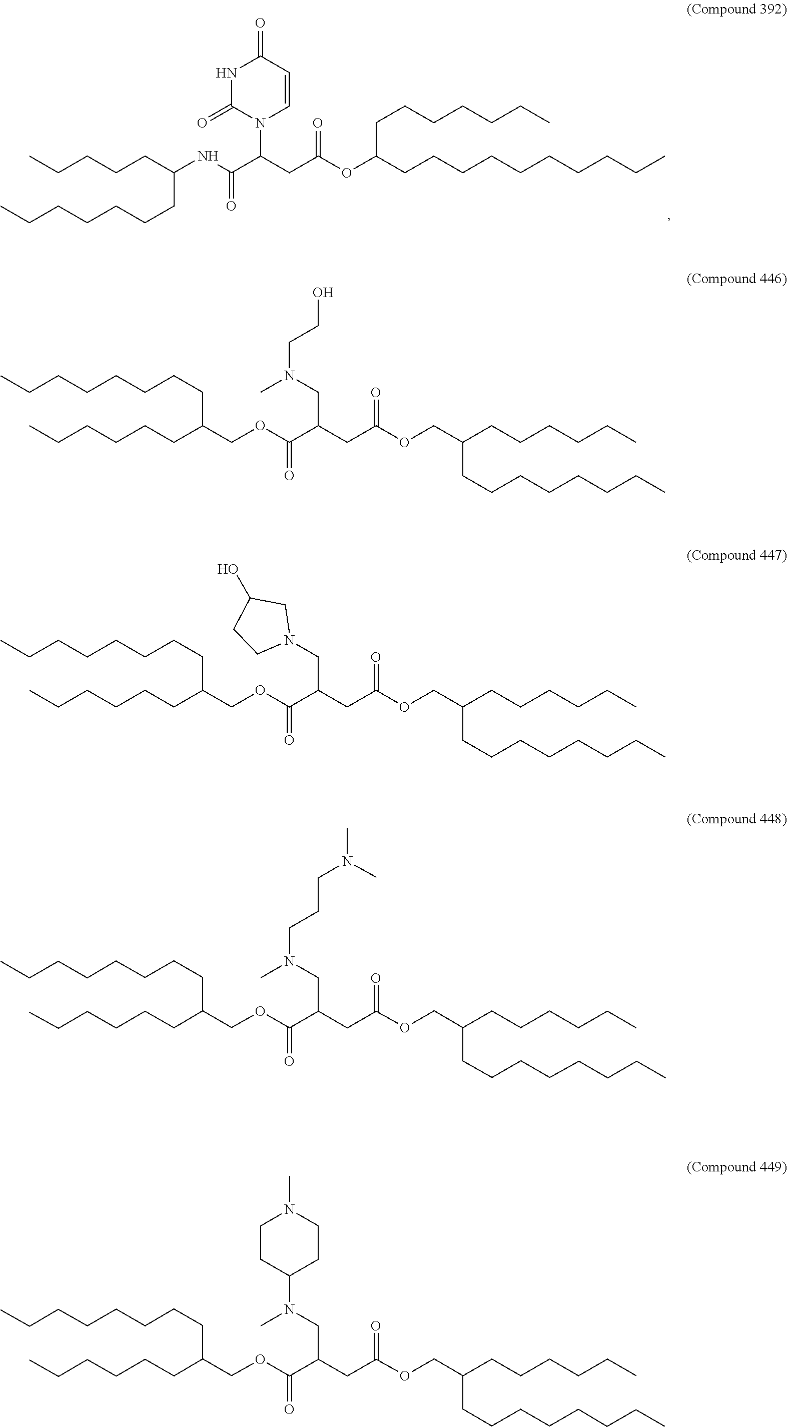

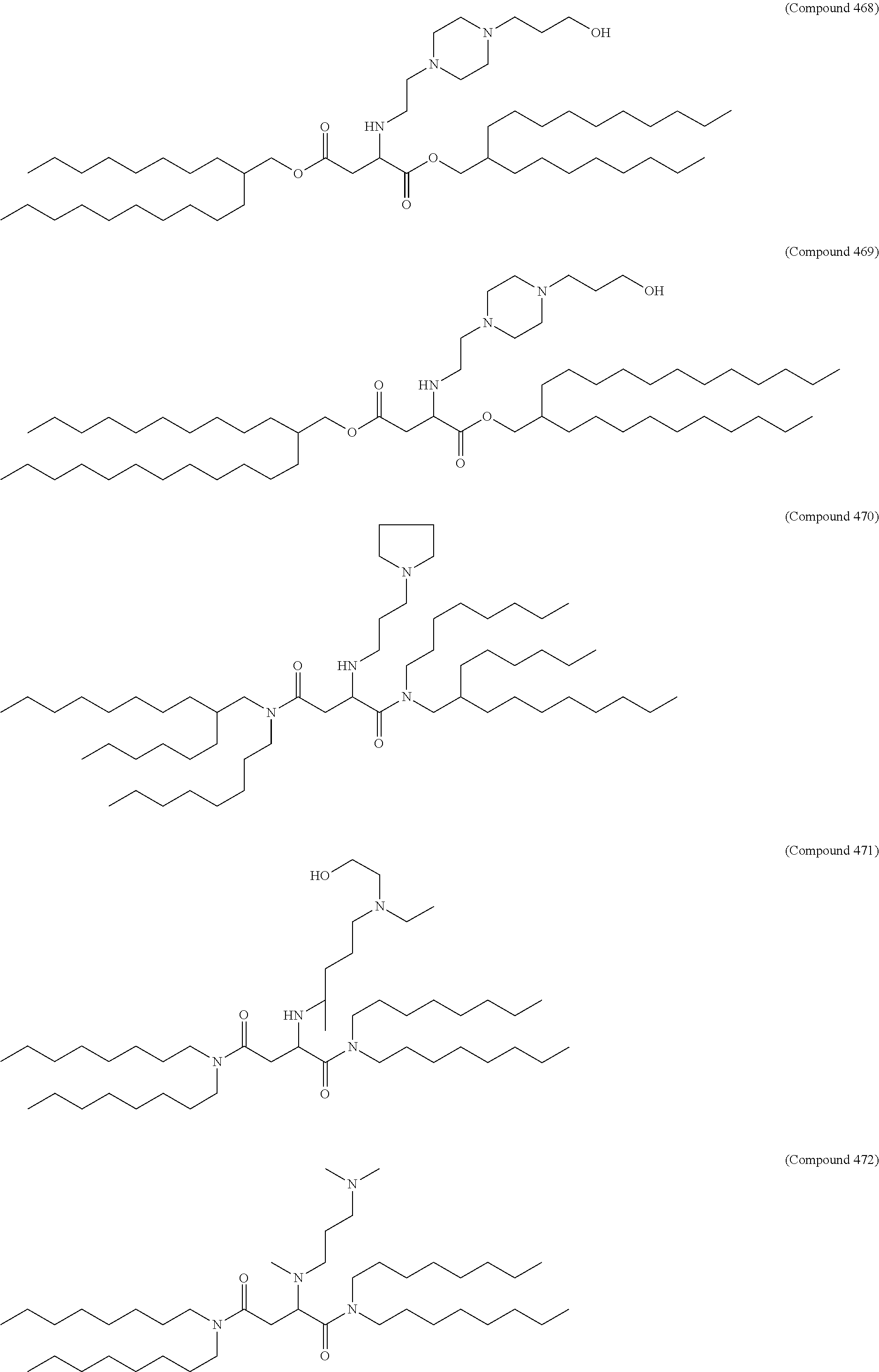

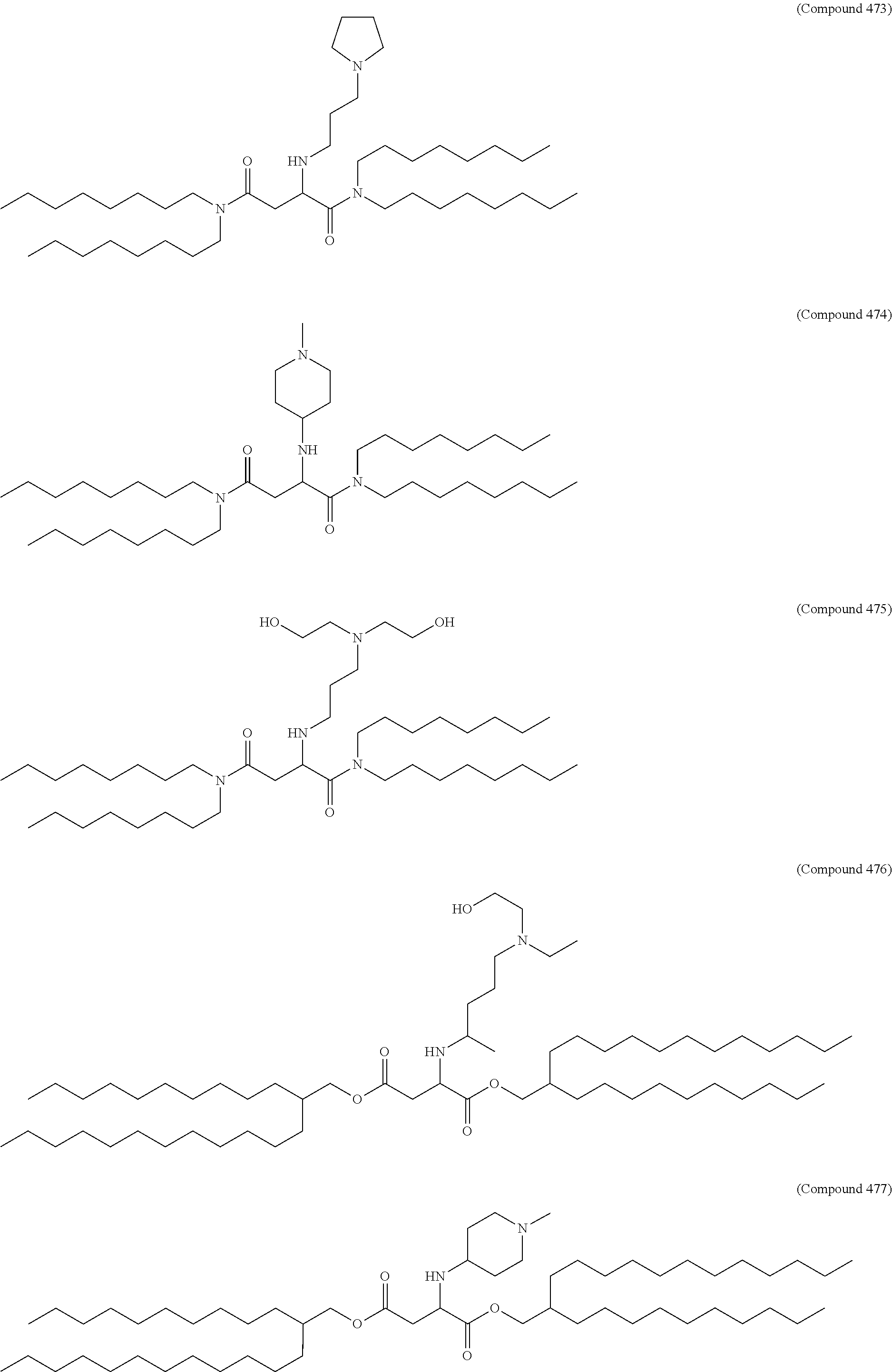

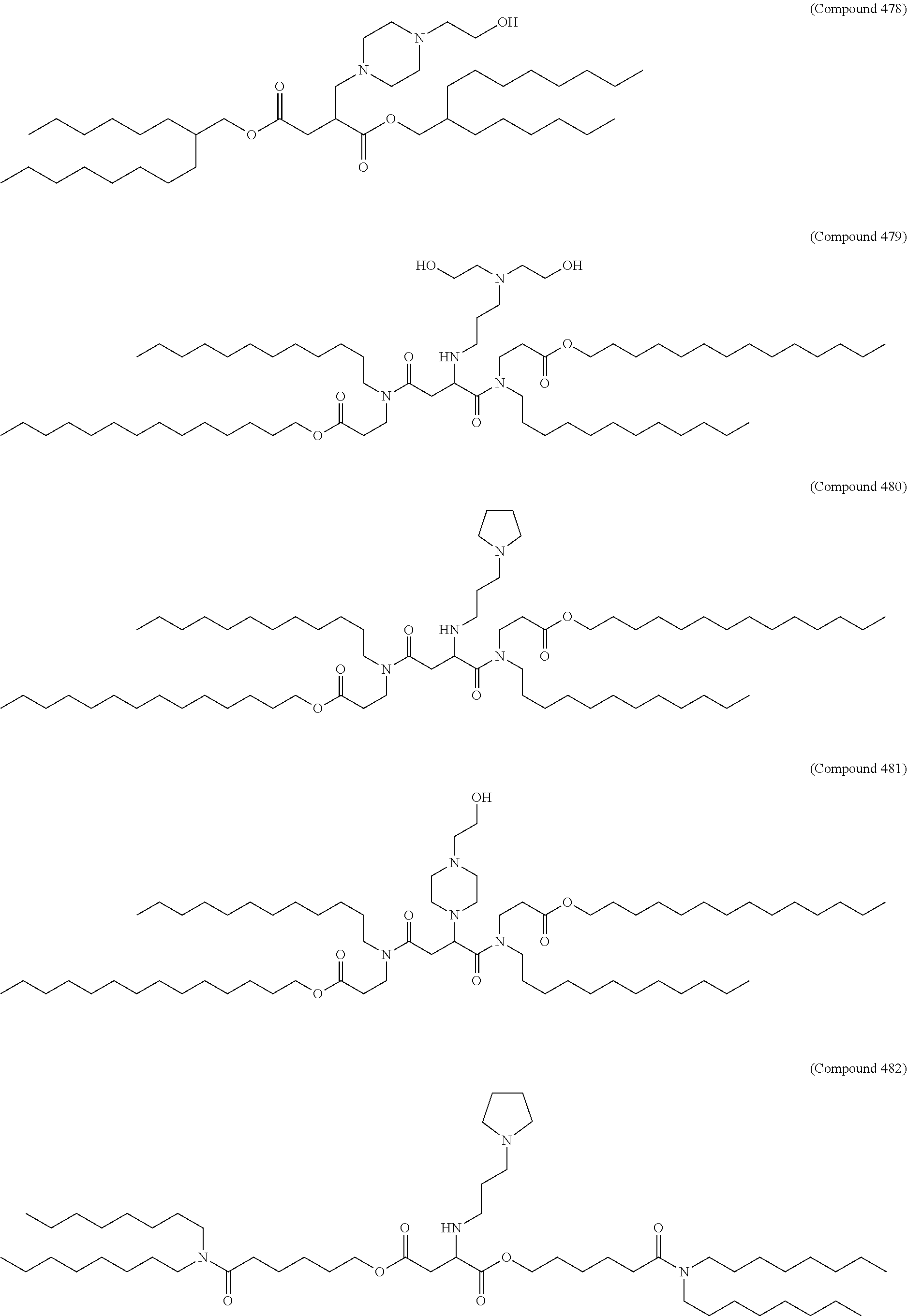

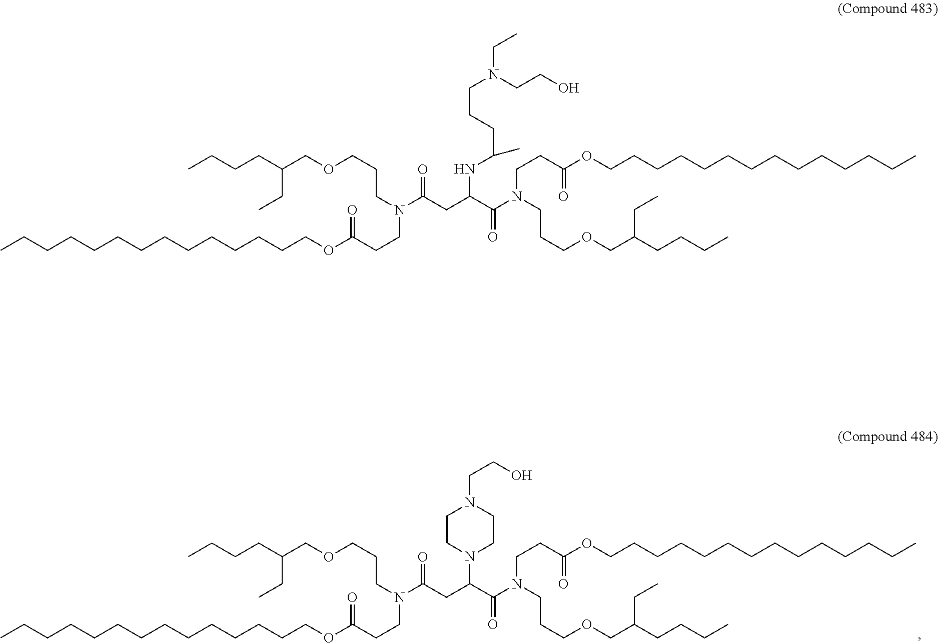

or salts or isomers thereof.

In another aspect of the disclosure, the compounds described herein are of Formula XI or a salt or isomer thereof

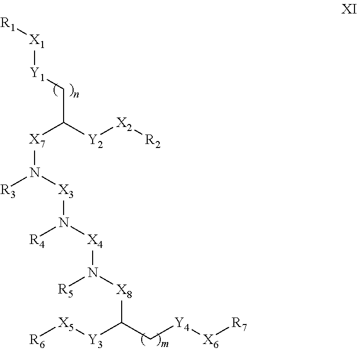

wherein:
R₁, R₂, R₃, R₄, R₅, R₆, and R₇ are the same or different and are independently selected from hydrogen, halogen, alkyl, alkenyl, alkynyl, carbocyclyl, heterocyclyl, aryl, and heteroaryl groups, each except hydrogen and halogen optionally substituted;
X₁, X₂, X₃, X₄, X₅ and X₆ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, alkynylenes, carbocyclyl, and a bond, wherein R at each occurrence is independently hydrogen, alkyl, alkenyl, alkynyl, carbocyclyl, or heterocyclyl;
X₇ and X₈ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, or alkynylene, and carbocycylyl, wherein R at each occurrence is independently hydrogen, alkyl, alkenyl, alkynyl, carbocyclyl, or heterocyclyl; or X₇ and X₈ are each independently a linear alkyl group comprising 0 to 10 methylene units;
Y₁, Y₂, Y₃, and Y₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, and an alkynylene wherein R at each occurrence is independently hydrogen, or an alkyl, alkenyl, alkynyl, carbocyclyl, heterocyclyl, group; and
n and m are independently selected from 0, 1, 2, 3, 4, 5, 6, 7, 8, 9, and 10,
wherein the longest chain of atoms in the compound is between 18 to 70 atoms.

In some embodiments, R₃ and R₄ may together with the atoms they are attached, form part of a 5- to 10-membered aromatic or non-aromatic heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₄ and R₅ may together with the atoms they are attached, form part of a 5- to 10-membered aromatic or non-aromatic heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₁, X₁, and Y₁ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₂, X₂, and Y₂ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₆, X₅, and Y₃ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₇, X₆, and Y₄ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₃, R₄, and R₅ are independently selected from methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, and icosyl, each optionally substituted by —OH, or alternatively R₃, R₄, and R₅ together make up a portion of a ring system.

In some embodiments, R₃ and R₄ make up a portion of a ring system.

In some embodiments, X₇ and X₈ are independently selected from oxygen atoms (—O—), sulfur atoms (—S—), substituted or unsubstituted nitrogen atoms (—NR—), alkenylenes, alkynylenes, cyclic alkylenes or heteroalkylenes, esters, thioesters, or amides.

In some embodiments, Y₁, Y₂, Y₃, and Y₄ are independently selected from oxygen atoms (—O—), sulfur atoms (—S—), substituted or unsubstituted nitrogen atoms (—NR—), esters, thioesters, amides, alkenylenes, alkynylenes, or cyclic alkylenes or heteroalkylenes.

In some embodiments, in the compounds of Formula XI:
R₁, R₂, R₃, R₄, R₅, R₆, and R₇ are the same or different and are independently selected from hydrogen, alkyl, alkenyl, alkynyl, carbocyclyl, heterocyclyl, aryl, and heteroaryl groups, each, except hydrogen, optionally substituted;
X₁, X₂, X₃, X₄, X₅ and X₆ are the same or different and are independently selected from —(C═O)O—, —O(C═O)—, —NR(C═O)—, —(C═O)NR—, —O—, —S—, —CH₂—, —NR—, alkylene, alkenylene, alkynylenes, carbocyclyl, and a bond, wherein R at each occurrence is independently hydrogen, alkyl, alkenyl, alkynyl, carbocyclyl, or heterocyclyl;
X₇ and X₈ are the same or different and are independently selected from —(C═O)O—, —O(C═O)—, —O—, —S—, —CH₂—, —NR—, alkylene, alkenylene, or alkynylene, carbocycylyl, and a bond, wherein R at each occurrence is independently hydrogen, alkyl, alkenyl, alkynyl, carbocyclyl, or heterocyclyl; or X₇ and X₈ are each independently a linear alkyl group comprising 0 to 5 methylene units;
Y₁, Y₂, Y₃, and Y₄ are the same or different and are independently selected from —(C═O)O—, —O(C═O)—, —NR(C═O)—, —(C═O)NR—, —O—, —S—, —CH₂—, alkenylene, and an alkynylene wherein R at each occurrence is independently hydrogen, or an alkyl, alkenyl, alkynyl, carbocyclyl, heterocyclyl, group; and
n and m are independently selected from 0, 1, 2, 3, 4, or 5,
wherein the longest chain of atoms in the compound is between 18 to 70 atoms.

In some embodiments, the compound of Formula XI may be selected from the Compounds of List 11:

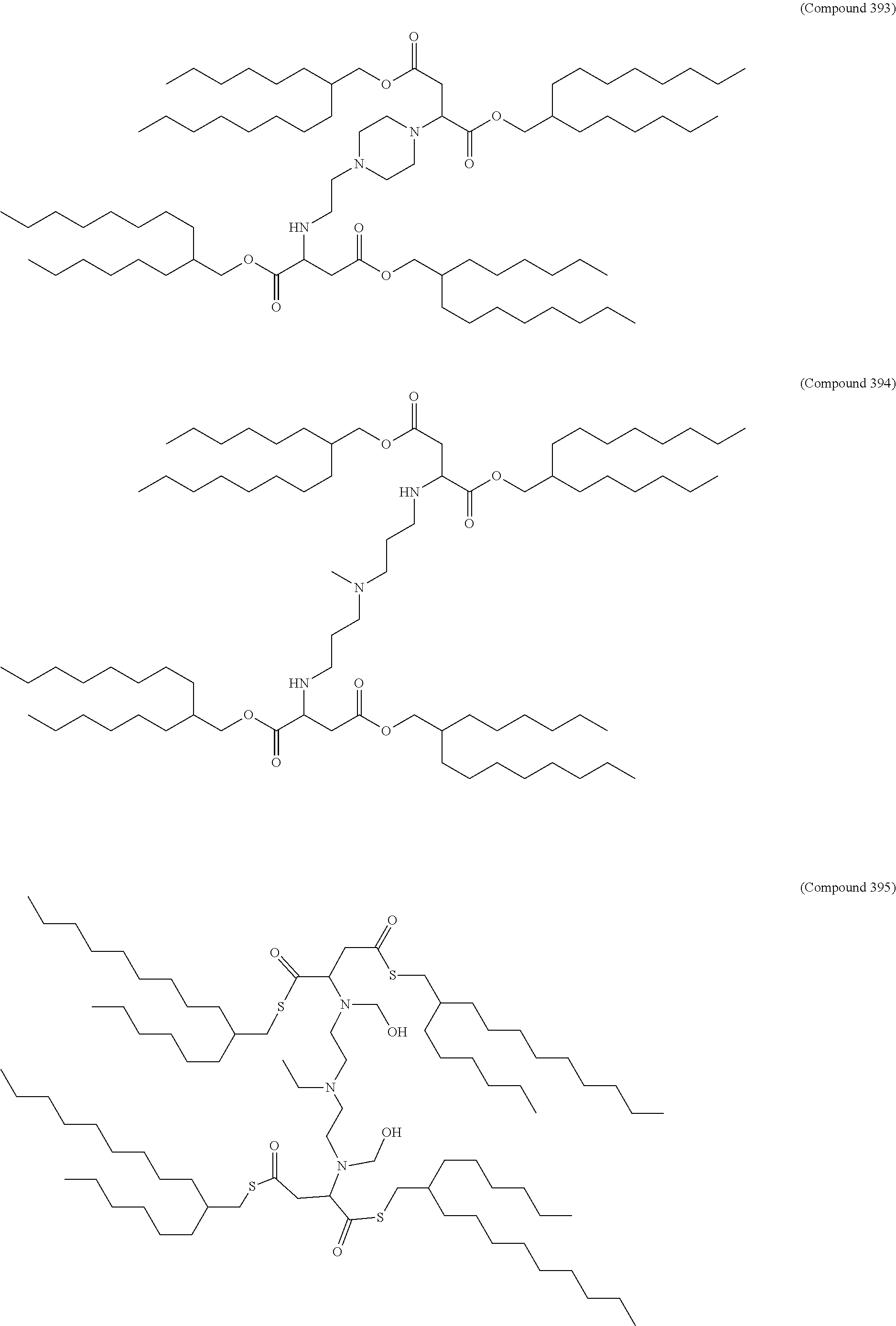

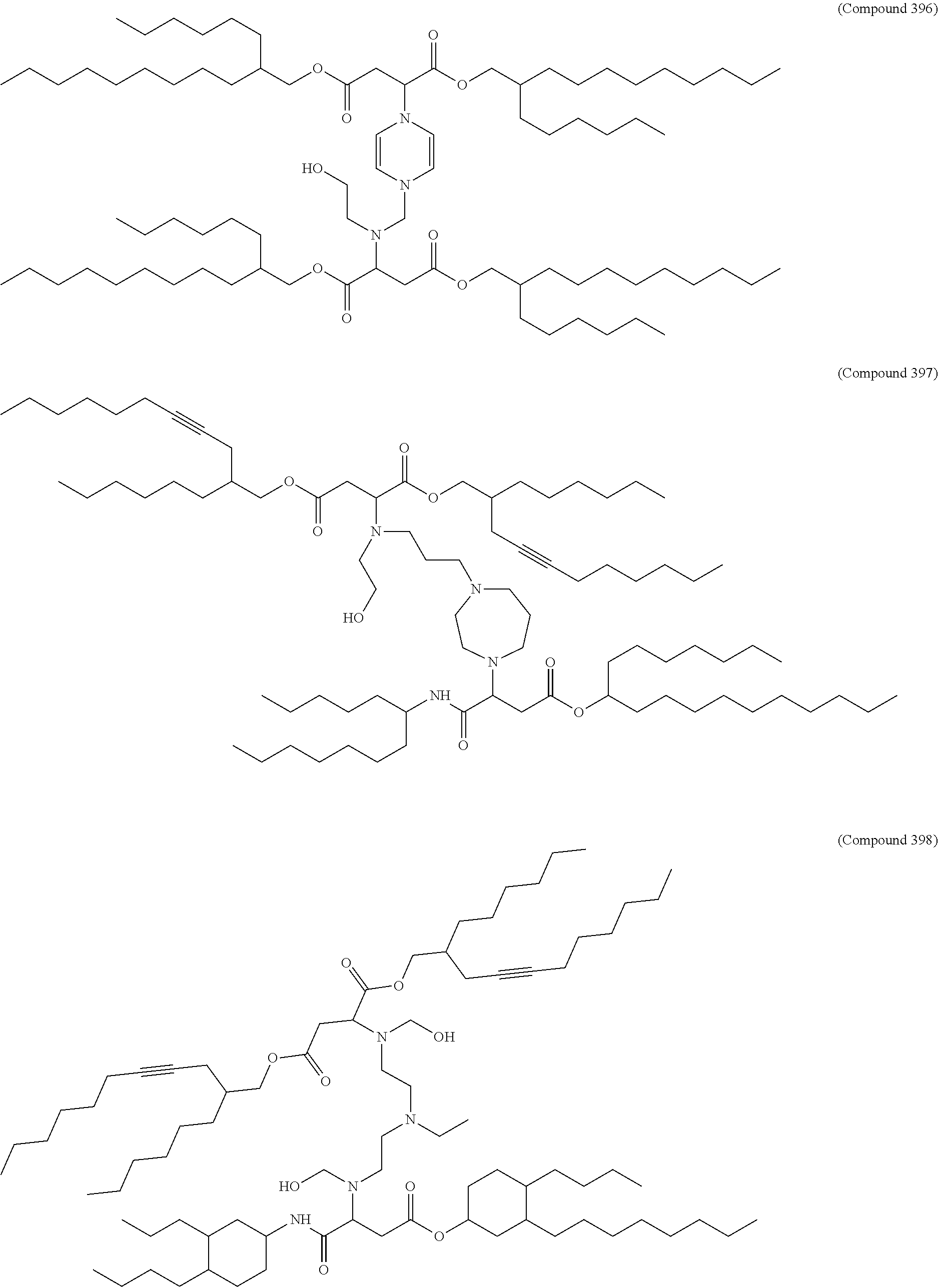

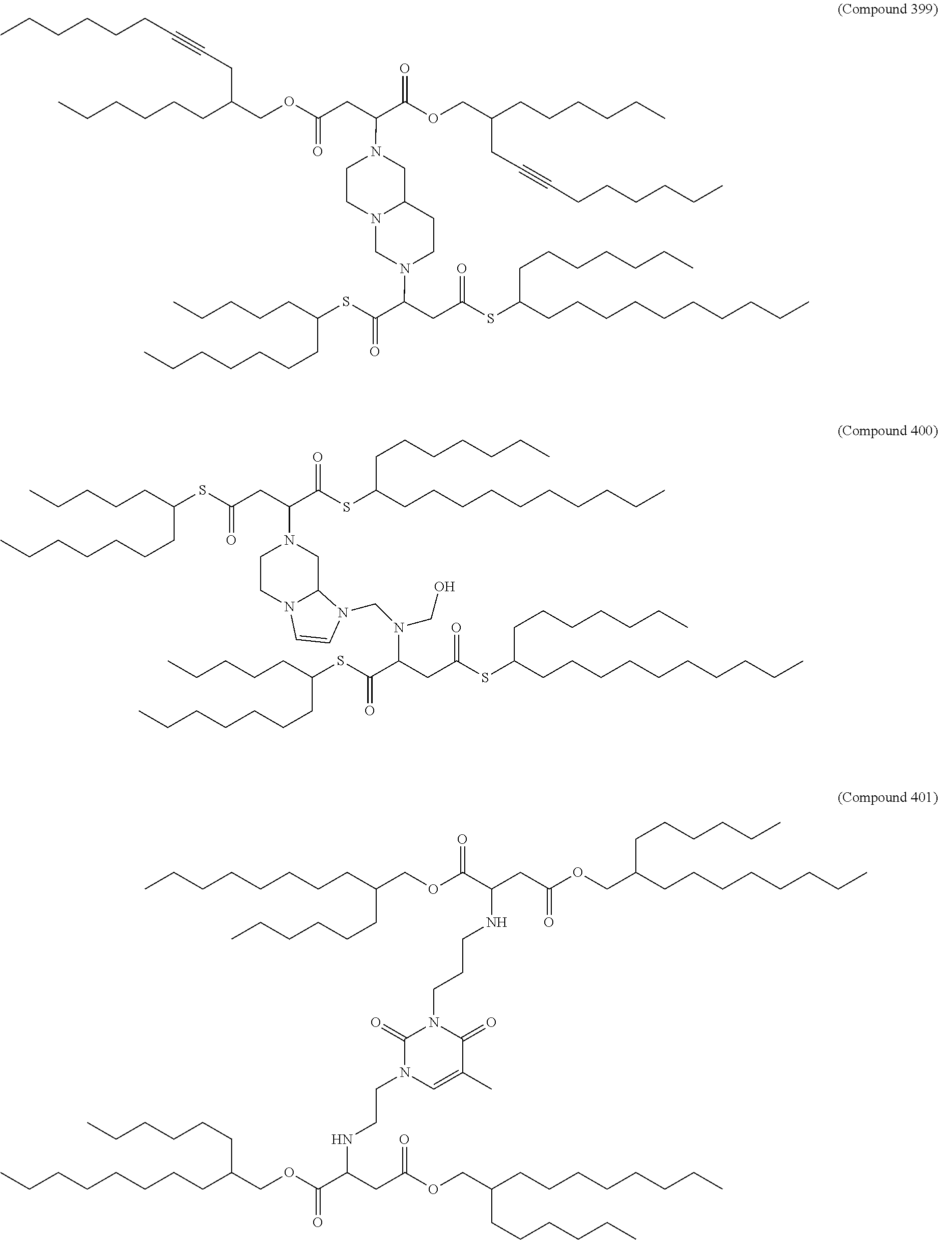

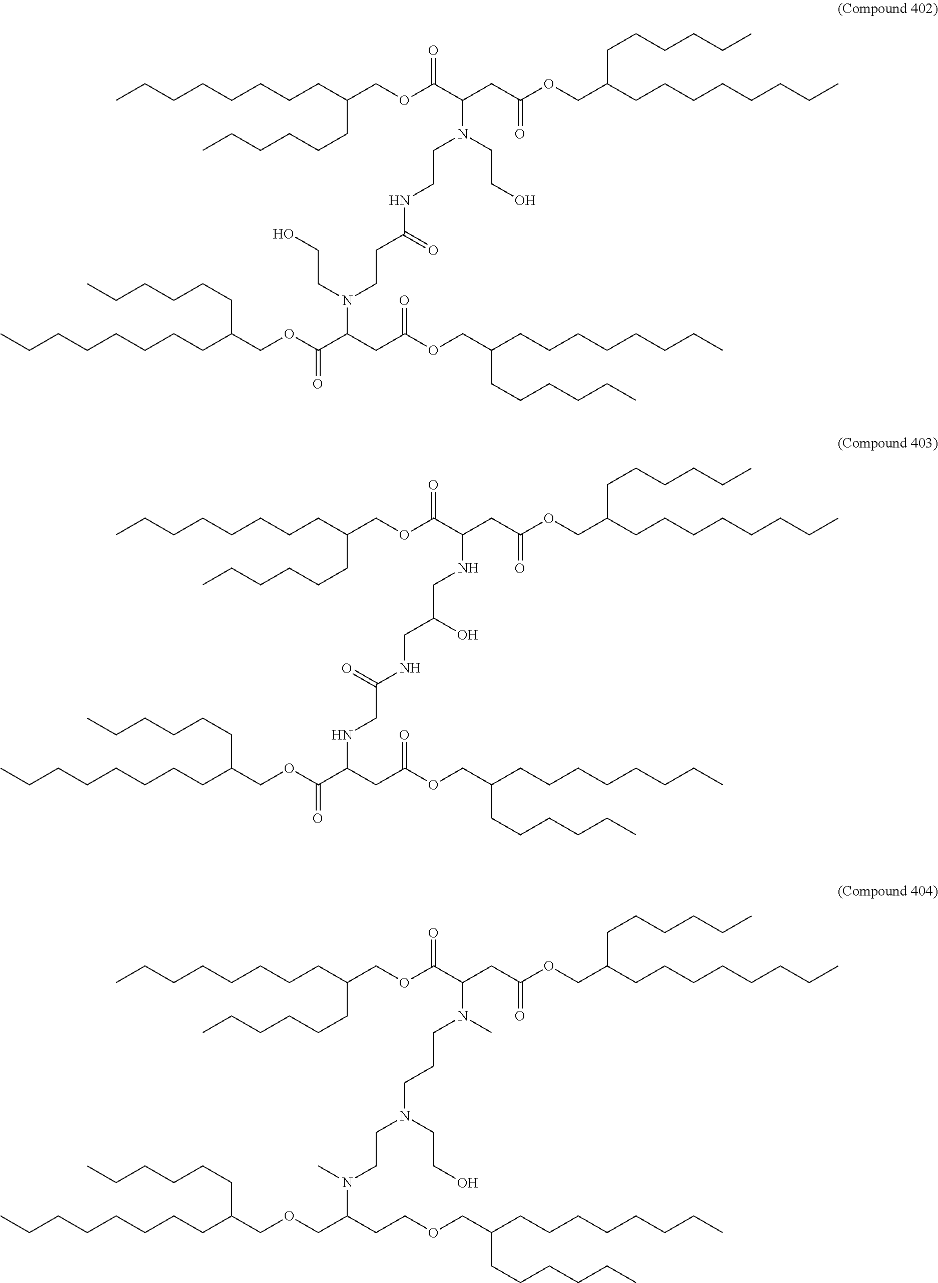

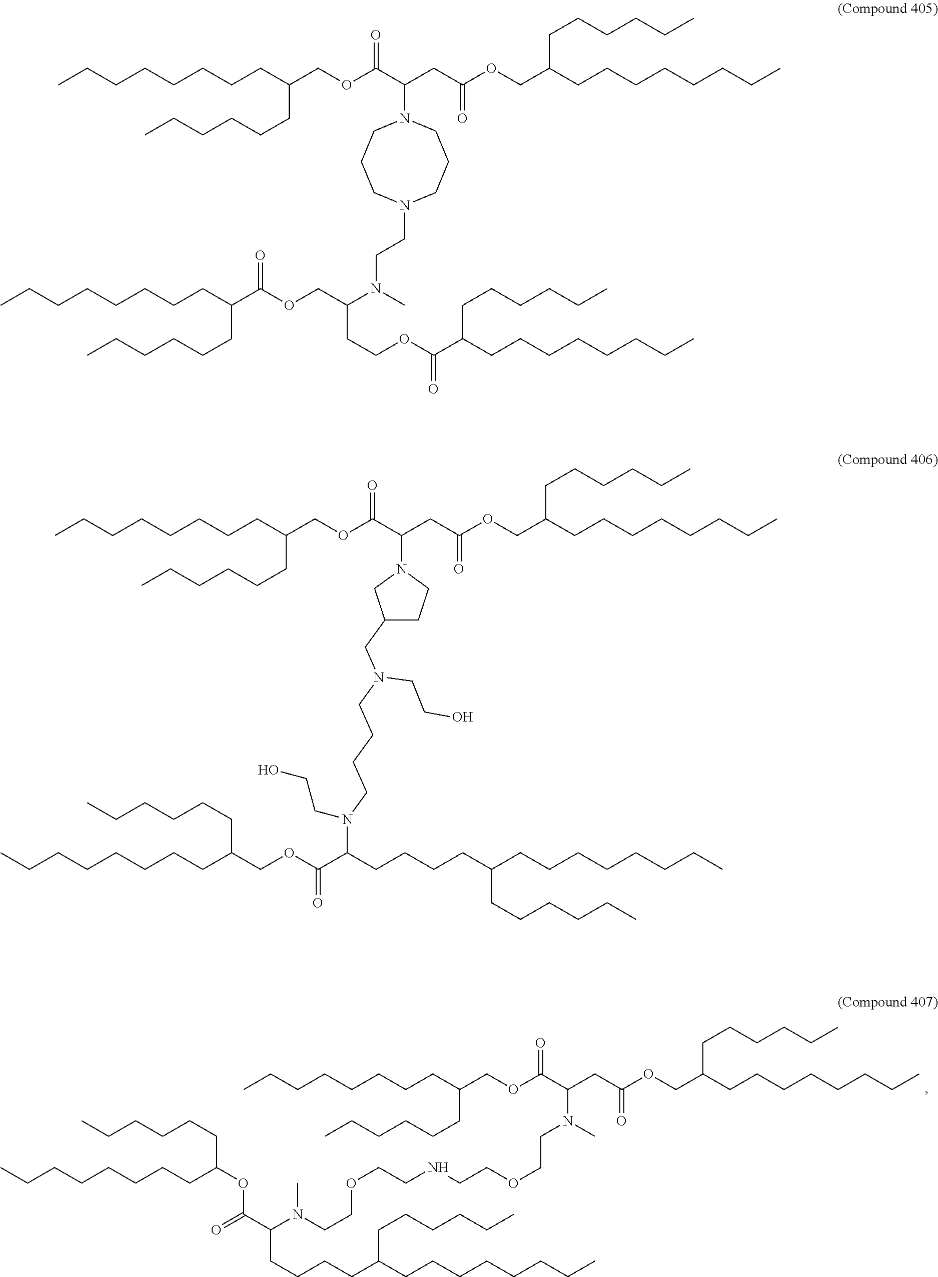

or salts or isomers thereof.

In another aspect of the disclosure, the compounds described herein are of Formula XII or a salt or isomer thereof

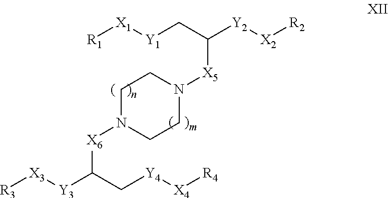

wherein:
R₁, R₂, R₃, and R₄ are the same or different and are independently selected from hydrogen, halogen, alkyl, alkenyl, alkynyl, carbocyclyl, heterocyclyl, aryl, and heteroaryl, each except hydrogen and halogen optionally substituted;
X₁, X₂, X₃, and X₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, alkynylene, and a bond, wherein R at each occurrent is independently hydrogen, alkyl, alkenyl, alkynyl, carbocyclyl, or heterocyclyl;
X₅ and X₆ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, alkynylene, and a bond, wherein R at each occurrence is independently hydrogen, or an alkyl, alkenyl, alkynyl, carbocyclyl, heterocyclyl, group; or X₅ and X₆ are each independently a linear alkyl group comprising 0 to 10 methylene units;
Y₁, Y₂, Y₃, and Y₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, alkynylene, and a bond, wherein R at each occurrence is independently hydrogen, or an alkyl, alkenyl, alkynyl, carbocyclyl, heterocyclyl, group;
the central ring structure is an optionally substituted aromatic, heteroaromatic, non-aromatic, or anti-aromatic ring system; and
n and m are independently selected from 0, 1, 2, 3, 4, 5, 6, 7, 8, 9, and 10,
wherein the longest chain of atoms in the compound is between 18 to 70 atoms.

In some embodiments, the central heterocyclic ring may contain more degrees of unsaturation (e.g., double or triple bonds) or be optionally substituted. In certain embodiments, the heterocyclic ring may be substituted such that the two substituents are not connected directly to the nitrogen of the ring, but rather to an atom adjacent to the nitrogen. In certain embodiments, the heterocyclic ring may be aromatic, such that there is no permanent cationic charge.

In some embodiments, R₁, X₁, and Y₁ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₂, X₂, and Y₂ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₃, X₃, and Y₃ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₄, X₄, and Y₄ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, X₁, X₂, X₃, and X₄ are independently selected from oxygen atoms (—O—), sulfur atoms (—S—), substituted or unsubstituted nitrogen atoms (—NR—), esters, thioesters, amides, cyclic alkylene or heteroalkylene, alkenylenes, or alkynylenes.

In some embodiments, X₅ and X₆ are independently selected from oxygen atoms, sulfur atoms, nitrogen atoms, alkenylenes, alkynylenes, cyclic alkylene or heteroalkylene moieties, or methylene chains.

In some embodiments, Y₁, Y₂, Y₃, and Y₄ are independently selected from oxygen atoms (—O—), sulfur atoms (—S—), substituted or unsubstituted nitrogen atoms (—NR—), esters, thioesters, amides, alkenylenes, alkynylenes, or cyclic alkylenes or heteroalkylenes.

In some embodiments, in the compounds of Formula XII:
R₁, R₂, R₃, and R₄ are the same or different and are independently selected from hydrogen, alkyl, alkenyl, alkynyl, carbocyclyl, heterocyclyl, each except hydrogen and halogen optionally substituted;
X₁, X₂, X₃, and X₄ are the same or different and are independently selected from —(C═O)O—, —O(C═O)—, —NR(C═O)—, —(C═O)NR—, —O—, —S—, —CH₂—, —NR—, alkenylene, alkynylene, and a bond, wherein R at each occurrent is independently hydrogen, alkyl, alkenyl, alkynyl, carbocyclyl, or heterocyclyl;
X₅ and X₆ are the same or different and are independently selected from —(C═O)O—, —O(C═O)—, —NR(C═O)—, —(C═O)NR—, —O—, —S—, —CH₂—, —NR—, alkenylene, alkynylene, and a bond, wherein R at each occurrence is independently hydrogen, or an alkyl, alkenyl, alkynyl, carbocyclyl, heterocyclyl, group; or X₅ and X₆ are each independently a linear alkyl group comprising 0 to 5 methylene units;
Y₁, Y₂, Y₃, and Y₄ are the same or different and are independently selected from —(C═O)O—, —O(C═O)—, —NR(C═O)—, —(C═O)NR—, —O—, —S—, —CH₂—, alkenylene, alkynylene, and a bond, wherein R at each occurrence is independently hydrogen, or an alkyl, alkenyl, alkynyl, carbocyclyl, heterocyclyl, group;
the central ring structure is an optionally substituted aromatic, heteroaromatic, non-aromatic, or anti-aromatic ring system; and
n and m are independently selected from 0, 1, 2, 3, 4, or 5,
wherein the longest chain of atoms in the compound is between 18 to 70 atoms.

In some embodiments, the compound of Formula XII may be selected from the compounds of List 12:

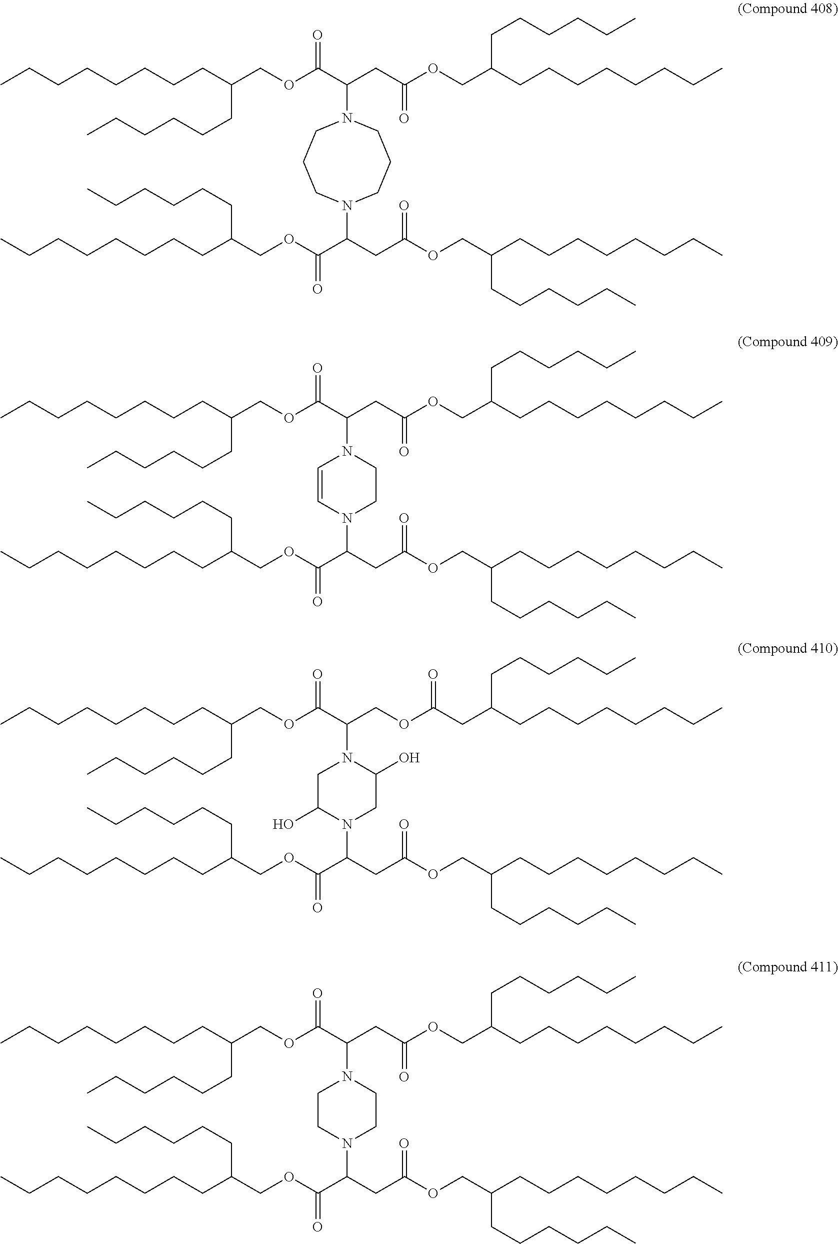

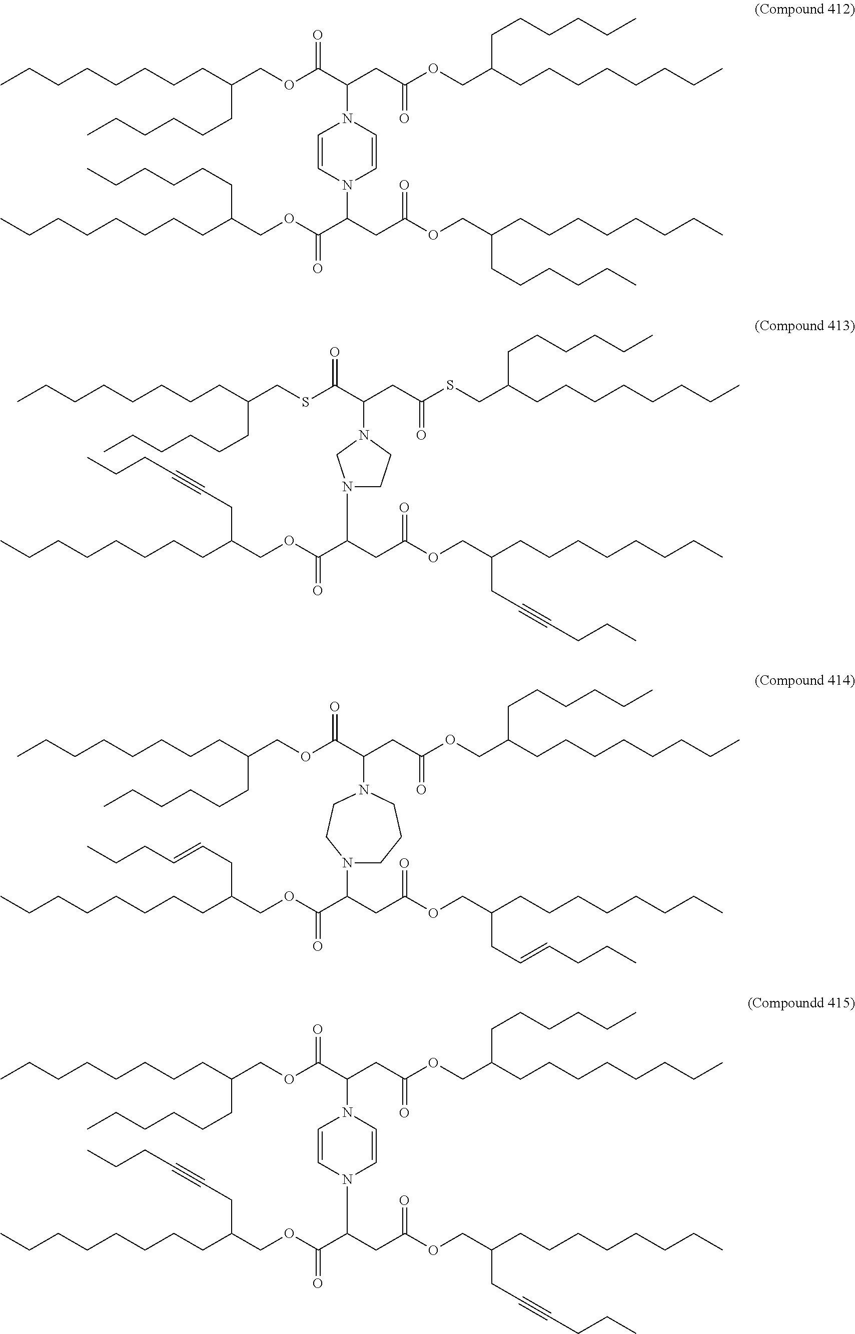

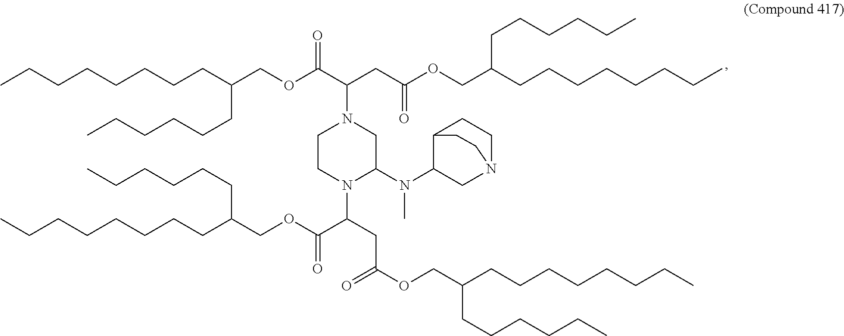 [SMILES: CCCCCCCCC(CCCCCC)COC(=O)CC(C(=O)OCC(CCCCCC)CCCCCCCC)N1CCN(C(CC(=O)OCC(CCCCCC)CCCCCCCC)C(=O)OCC(CCCCCC)CCCCCCCC)C(N(C)C2CN3CCC2CC3)C1]

or salts or isomers thereof.

In another aspect of the invention, the compounds described herein are of Formula XIII or a salt or isomer thereof

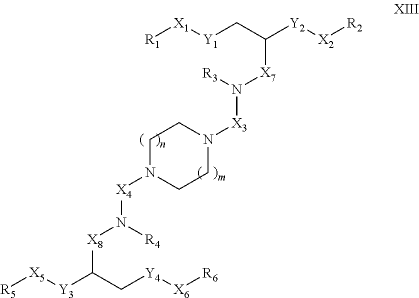

wherein:
R₁, R₂, R₃, R₄, R₅, and R₆ are the same or different and are independently selected from hydrogen, halogen, alkyl, alkenyl, alkynyl, carbocyclyl, heterocyclyl, aryl, and heteroaryl, each except hydrogen and halogen optionally substituted;
X₁, X₂, X₃, X₄, X₅, and X₆ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, alkynylene, and cyclic alkylene or heteroalkylenes, wherein R at each occurrence is independently hydrogen, or an alkyl, alkenyl, alkynyl, carbocyclyl, heterocyclyl, group;
X₇ and X₈ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, alkynylene, and cyclic alkylene or heteroalkylenes, wherein R is independently hydrogen, or an alkyl, alkenyl, alkynyl, carbocyclyl, heterocyclyl, group; or X₇ and X₈ are each a linear alkyl group comprising 0 to 10 methylene units;
Y₁, Y₂, Y₃, and Y₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, alkynylenes, and cyclic alkylene or heteroalkylenes, wherein R at each occurrence is independently hydrogen, or an alkyl, alkenyl, alkynyl, carbocyclyl, heterocyclyl, group;
the central ring structure is an optionally substituted aromatic, heteroaromatic, non-aromatic, or anti-aromatic ring system; and
n and m are independently selected from 0, 1, 2, 3, 4, 5, 6, 7, 8, 9, and 10,
wherein the longest chain of atoms in the compound is between 18 to 70 atoms.

In some embodiments, the central heterocyclic ring may contain more degrees of unsaturation (e.g., double or triple bonds) or be optionally substituted. In certain embodiments, the heterocyclic ring may be substituted such that the two substituents are not connected directly to the nitrogen of the ring, but rather to an atom adjacent to the nitrogen. In certain embodiments, the heterocyclic ring may be aromatic, such that there is no permanent cationic charge.

In some embodiments, R₁, X₁, and Y₁ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₂, X₂, and Y₂ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₅, X₅, and Y₃ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₆, X₆, and Y₄ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₃ and X₃ may together with the atoms they attach form part of a 3- to 10-membered aromatic or non-aromatic heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₄ and X₄ may together with the atoms they attach form part of a 3- to 10-membered aromatic or non-aromatic heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₃ and R₄ are independently methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl, each optionally substituted by —OH;

In some embodiments, X₁, X₂, X₅, and X₆ are independently selected from —(C═O)O—, —O(C═O)—, —NR(C═O)—, —(C═O)NR—, —O—, —S—, —NR—, alkenylenes, alkynylenes, or cyclic alkylene or heteroalkylenes, wherein R at each occurrence is independently hydrogen, or an alkyl, alkenyl, alkynyl, carbocyclyl, heterocyclyl, group.

In some embodiments, X₃ and X₄ are independently selected from —(C═O)O—, —O(C═O)—, —NR(C═O)—, —(C═O)NR—, —O—, —S—, —NR—, alkenylenes, alkynylenes, or cyclic alkyl or heteroalkyls, wherein R at each occurrence is independently hydrogen, or an alkyl, alkenyl, alkynyl, carbocyclyl, heterocyclyl, group.

In some embodiments, X₇ and X₈ are independently selected from —(C═O)O—, —O(C═O)—, —NR(C═O)—, —(C═O)NR—, —O—, —S—, —NR—, alkenylenes, alkynylenes, or cyclic alkylene or heteroalkylenes, or methylene chains, wherein R at each occurrence is independently hydrogen, or an alkyl, alkenyl, alkynyl, carbocyclyl, heterocyclyl, group.

In some embodiments, Y₁, Y₂, Y₃, and Y₄ are independently selected from —(C═O)O—, —O(C═O)—, —NR(C═O)—, —(C═O)NR—, —O—, —S—, —NR—, alkenylenes, alkynylenes, or cyclic alkylene or heteroalkylenes, wherein R at each occurrence is independently hydrogen, or an alkyl, alkenyl, alkynyl, carbocyclyl, heterocyclyl, group.

In some embodiments, in the compounds of Formula XIII:
R₁, R₂, R₃, R₄, R₅, and R₆ are the same or different and are independently selected from hydrogen, alkyl, alkenyl, alkynyl, carbocyclyl, and heterocyclyl, each except hydrogen and halogen optionally substituted;
X₁, X₂, X₃, X₄, X₅, and X₆ are the same or different and are independently selected from —(C═O)O—, —O(C═O)—, —NR(C═O)—, —(C═O)NR—, —O—, —S—, —CH₂—, —NR—, alkenylene, alkynylene, and cyclic alkylene or heteroalkylenes, wherein R at each occurrence is independently hydrogen, or an alkyl, alkenyl, alkynyl, carbocyclyl, heterocyclyl, group;
X₇ and X₈ are the same or different and are independently selected from —(C═O)O—, —O(C═O)—, —NR(C═O)—, —(C═O)NR—, —O—, —S—, —CH₂—, —NR—, alkenylene, alkynylene, and cyclic alkylene or heteroalkylenes, wherein R is independently hydrogen, or an alkyl, alkenyl, alkynyl, carbocyclyl, heterocyclyl, group; or X₇ and X₈ are each a linear alkyl group comprising 0 to 10 methylene units;
Y₁, Y₂, Y₃, and Y₄ are the same or different and are independently selected from —(C═O)O—, —O(C═O)—, —NR(C═O)—, —(C═O)NR—, —O—, —S—, —CH₂—, alkenylene, alkynylenes, and cyclic alkylene or heteroalkylenes, wherein R at each occurrence is independently hydrogen, or an alkyl, alkenyl, alkynyl, carbocyclyl, heterocyclyl, group;
the central ring structure is an optionally substituted aromatic, heteroaromatic, non-aromatic, or anti-aromatic ring system; and
n and m are independently selected from 0, 1, 2, 3, 4, or 5,
wherein the longest chain of atoms in the compound is between 18 to 70 atoms.

In some embodiments, the compound of Formula XIII may be selected from the Compounds of List 13:

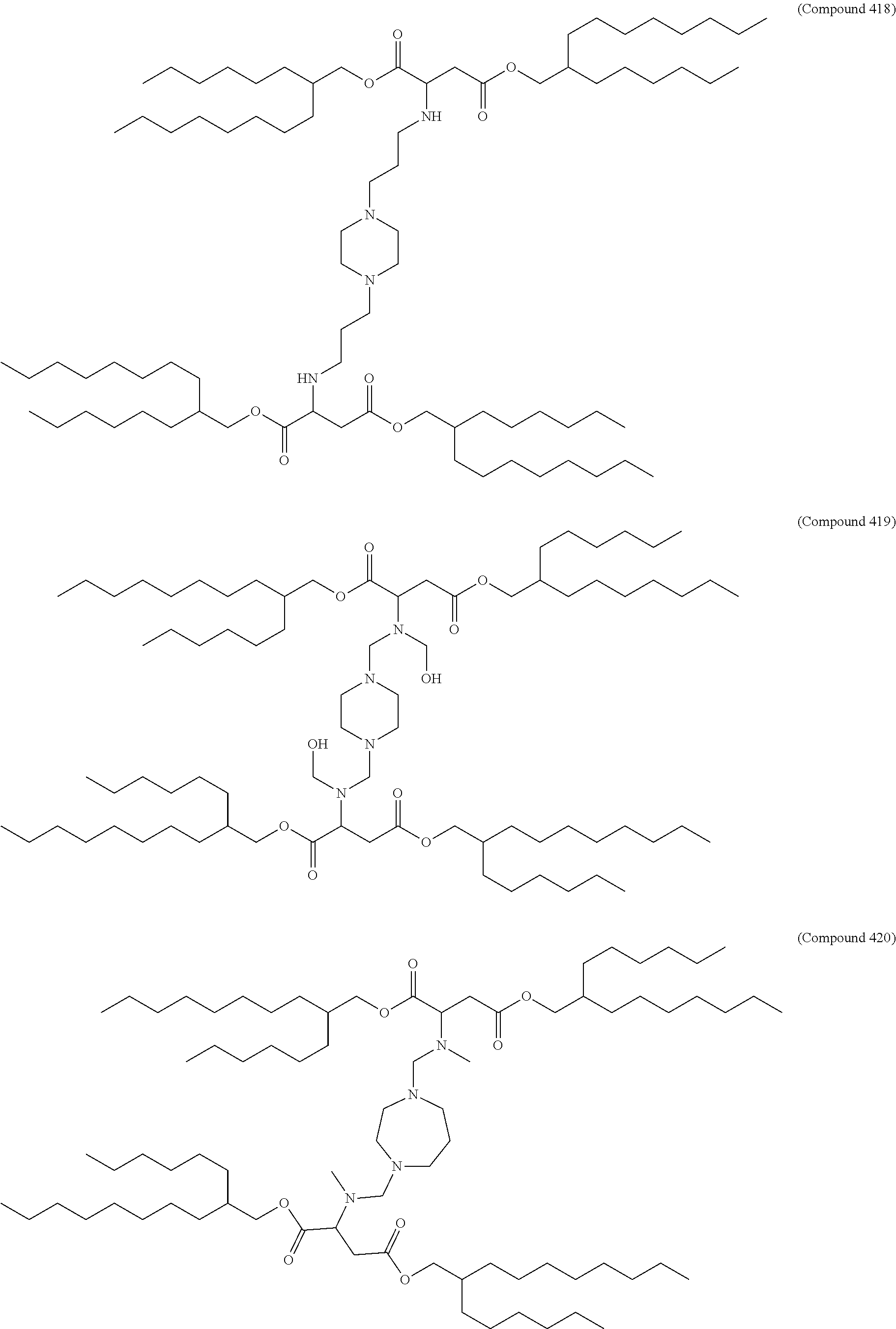

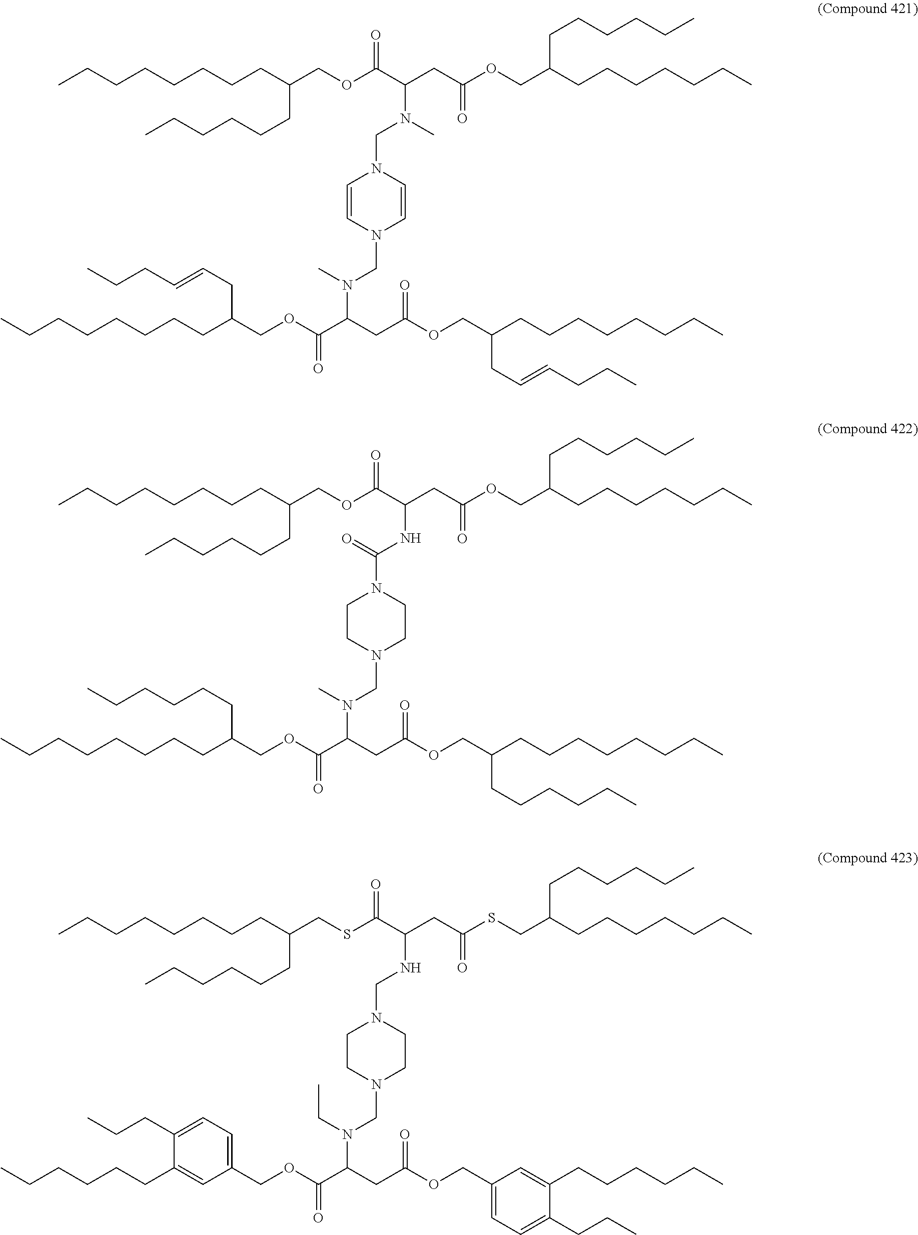

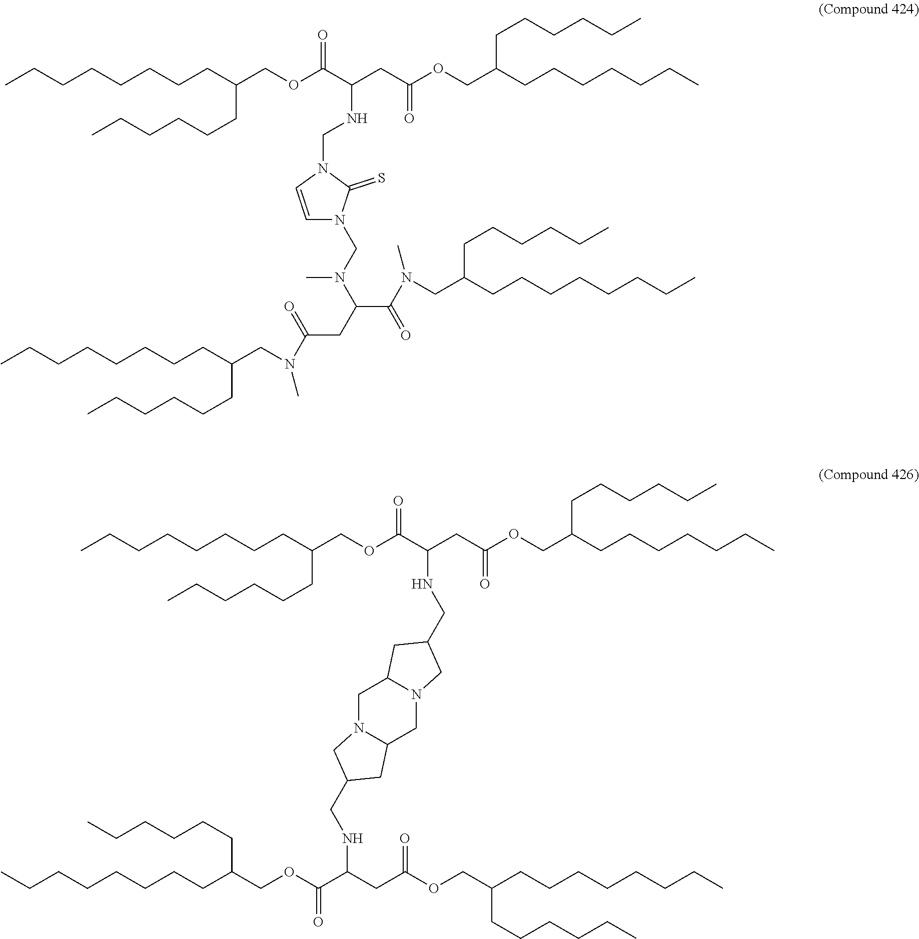

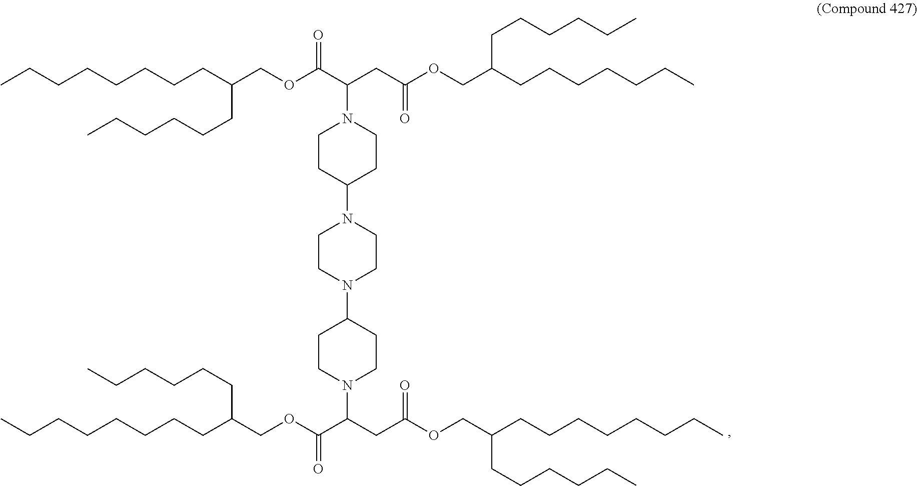 [SMILES: CCCCCCCCC(CCCCCC)COC(=O)CC(C(=O)OCC(CCCCCC)CCCCCCCC)N1CCC(N2CCN(C3CCN(C(CC(=O)OCC(CCCCCC)CCCCCCC)C(=O)OCC(CCCCCC)CCCCCCCC)CC3)CC2)CC1]

or salts or isomers thereof.

In another aspect of the invention, the compounds described herein are of Formula XIV or a salt or isomer thereof

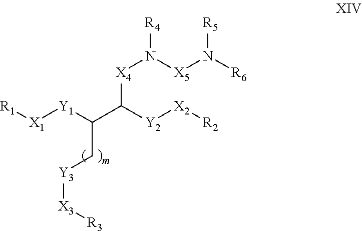

wherein:
R₁, R₂, R₃, R₄, R₅, and R₆ the same or different and are independently selected from hydrogen, halogen, alkyl, alkenyl, alkynyl, carbocyclyl, heterocyclyl, aryl, and heteroaryl, each except hydrogen and halogen optionally substituted;
X₁, X₂, X₃, X₄, and X₅ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, alkynylene, cycloalkylene, and heterocyclylene, wherein R is independently hydrogen, alkyl, alkenyl, alkynyl, carbocyclyl, and heterocyclyl;
Y₁, Y₂, and Y₃ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkylene, alkenylene, alkynylene, cycloalkylene, and heterocyclylene, wherein R is independently hydrogen, alkyl, alkenyl, alkynyl, carbocyclyl, or heterocyclyl; and
m is selected from 0, 1, 2, 3, 4, 5, 6, 7, 8, 9, and 10,
wherein the longest chain of atoms in the compound is between 18 to 70 atoms.

In some embodiments, R₁, X₁, and Y₁ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₂, X₂, and Y₂ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₃, X₃, and Y₃ may together form part of a 3- to 10-membered aromatic or non-aromatic carbocycle or heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the carbocycle or heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₄, X₅, and Y₅ may together form part of a 4- to 10-membered aromatic or non-aromatic heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, X₄ and X₅ may together with the atoms they are attached for part of a 3- to 10-membered aromatic or non-aromatic heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, R₅ and R₆ may together form part of a 3- to 10-membered aromatic or non-aromatic heterocycle having one or more heteroatoms selected from N, O, S, and P, which can optionally be part of a functional group such as, but not limited to, ethers, sulfides, disulfides, esters, sulfonates, esters, thioesters, sulfones, sulfoxides, amines, amides, carbamates, carbonates, alkenes, alkynes, or imines. In certain embodiments, the heterocycle formed may optionally be substituted with one or more substituents, such as, but not limited to, alkyl groups, alkenyl groups, alkynyl groups, C₃₋₇ carbocyclic groups, 3- to 8-membered heterocyclic groups, —OH, —SH, —OR, —SR, —NR₂, NHR, -oxo, —O—CO—, —NR—CO—, or other functional groups.

In some embodiments, the 2-carbon chain between Y₁ and Y₂ may contain certain degrees of unsaturation (e.g., an alkene or alkyne).

In some embodiments, X₁, X₂, and X₃ are independently selected from —(C═O)O—, O(C═O)—, —NR(C═O)—, —(C═O)NR—, —O—, —S—, —NR—, alkenylene, alkynylene, cycloalkylene, and heterocyclylene, wherein R at each occurrence is independently hydrogen, or an alkyl, alkenyl, alkynyl, carbocyclyl, heterocyclyl, group.

In some embodiments, X₄ and X₅ are independently selected from —(C═O)O—, O(C═O)—, —NR(C═O)—, —(C═O)NR—, —O—, —S—, —NR—, alkylene, alkenylene, alkynylene, cycloalkylene, heterocyclylene, arylene, and heteroarylene rings, wherein R at each occurrence is independently hydrogen, or an alkyl, alkenyl, alkynyl, carbocyclyl, heterocyclyl, group.

In some embodiments, Y₁, Y₂, and Y₃ are independently selected from —(C═O)O—, O(C═O)—, —NR(C═O)—, —(C═O)NR—, —O—, —S—, —NR—, alkenyene, alkynylene, cycloalkylene, and heterocyclylene, wherein R at each occurrence is independently hydrogen, or an alkyl, alkenyl, alkynyl, carbocyclyl, heterocyclyl, group.

In some embodiments, in the compounds of Formula XIV:
R₁, R₂, R₃, R₄, R₅, and R₆ the same or different and are independently selected from hydrogen, alkyl, alkenyl, alkynyl, carbocyclyl, and heterocyclyl, each, except hydrogen, optionally substituted;
X₁, X₂, X₃, X₄, and X₅ are the same or different and are independently selected from —(C═O)O—, —O(C═O)—, —NR(C═O)—, —(C═O)NR—, —O—, —S—, —CH₂—, —NR—, alkylene, alkenylene, alkynylene, cycloalkylene, and heterocyclylene, wherein R is independently hydrogen, alkyl, alkenyl, alkynyl, carbocyclyl, and heterocyclyl;
Y₁, Y₂, and Y₃ are the same or different and are independently selected from —(C═O)O—, —O(C═O)—, —NR(C═O)—, —(C═O)NR—, —O—, —S—, —CH₂—, alkylene, alkenylene, alkynylene, cycloalkylene, and heterocyclylene, wherein R is independently hydrogen, alkyl, alkenyl, alkynyl, carbocyclyl, or heterocyclyl; and
m is selected from 0, 1, 2, 3, 4, or 5,
wherein the longest chain of atoms in the compound is between 18 to 70 atoms.

In some embodiments, the compound of Formula XIV may be selected from the Compounds of List 14:

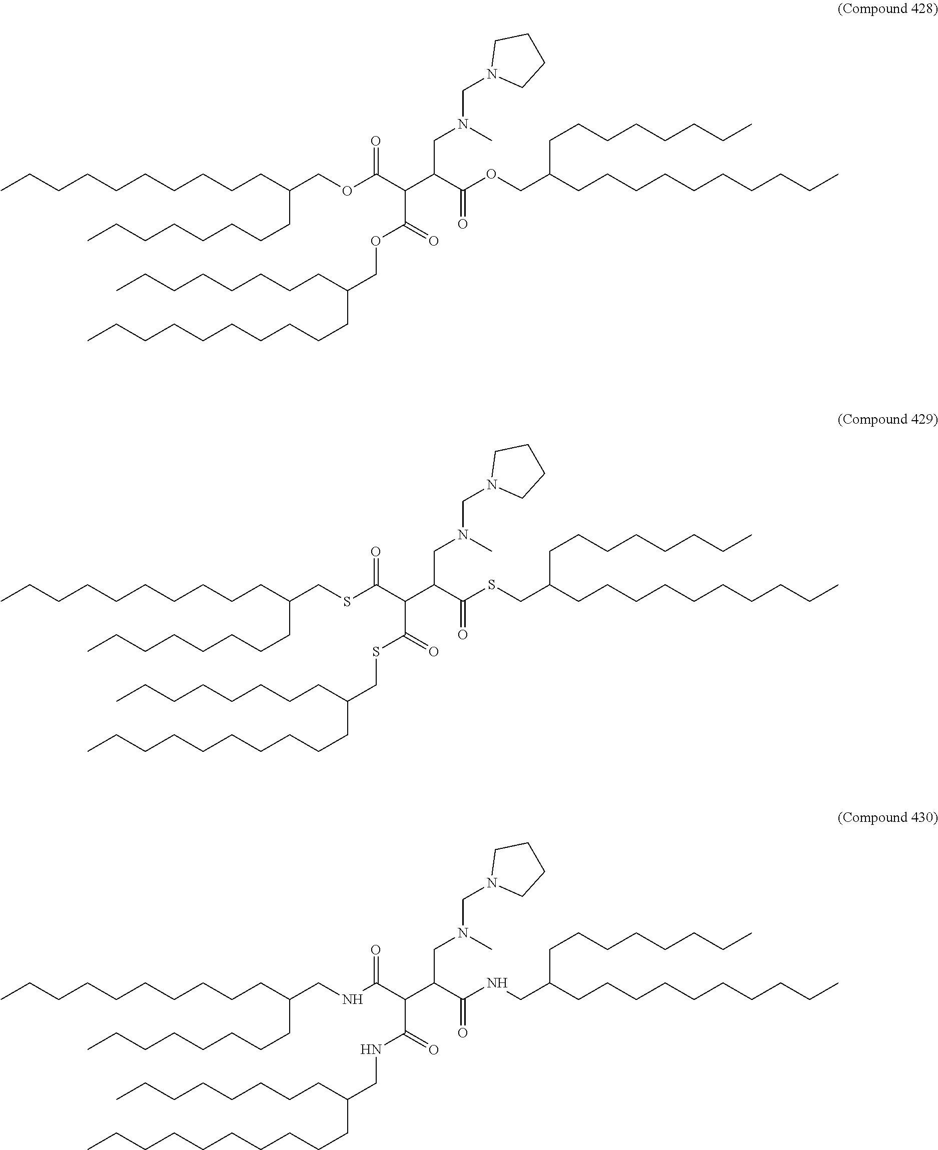

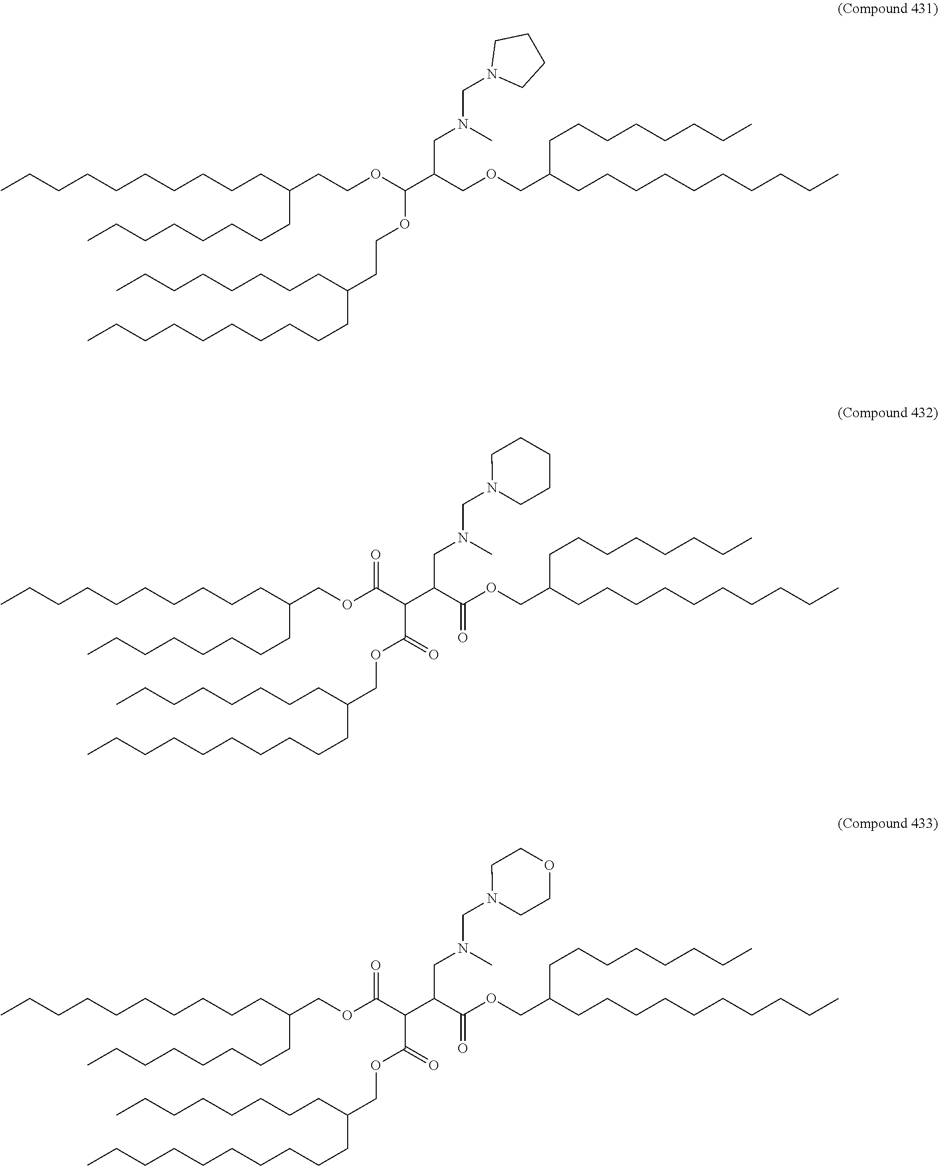

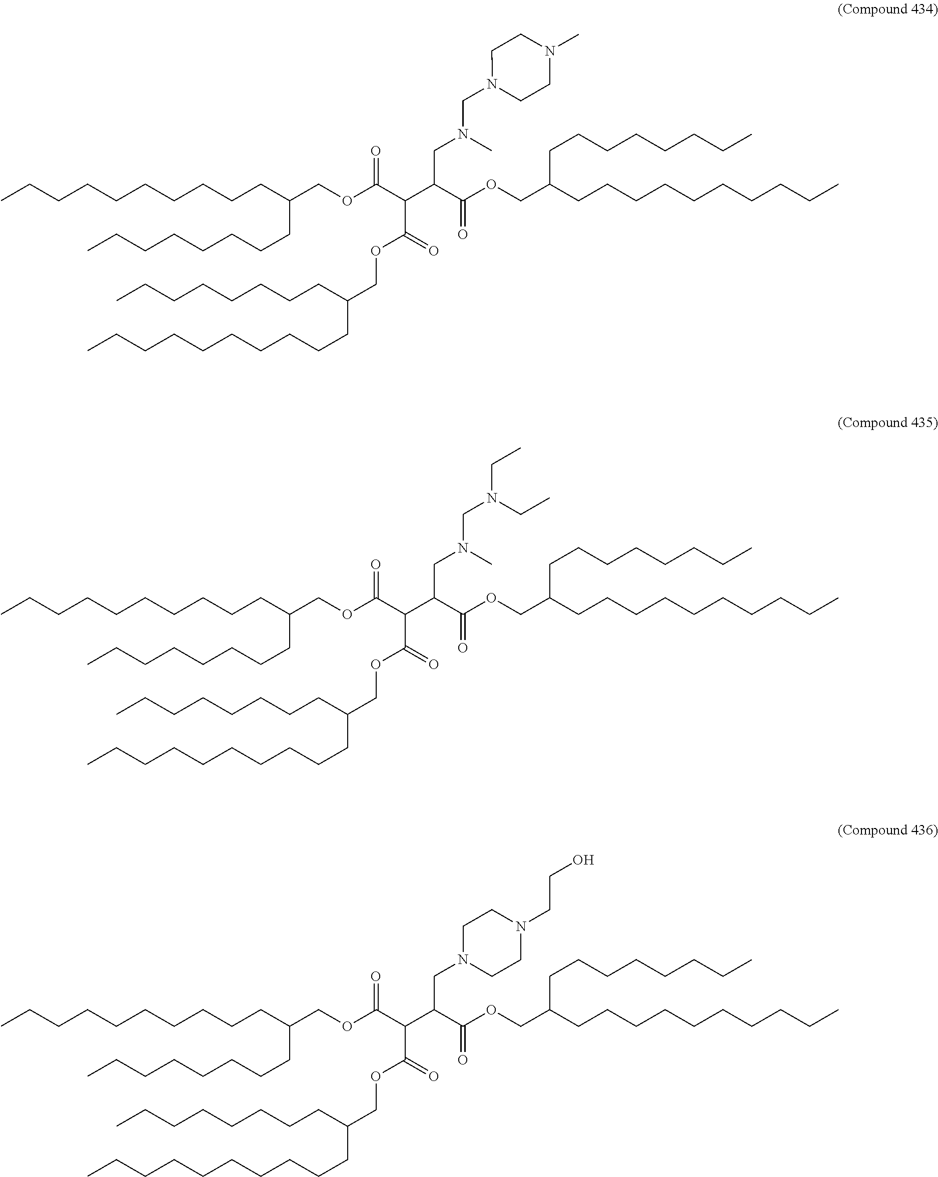

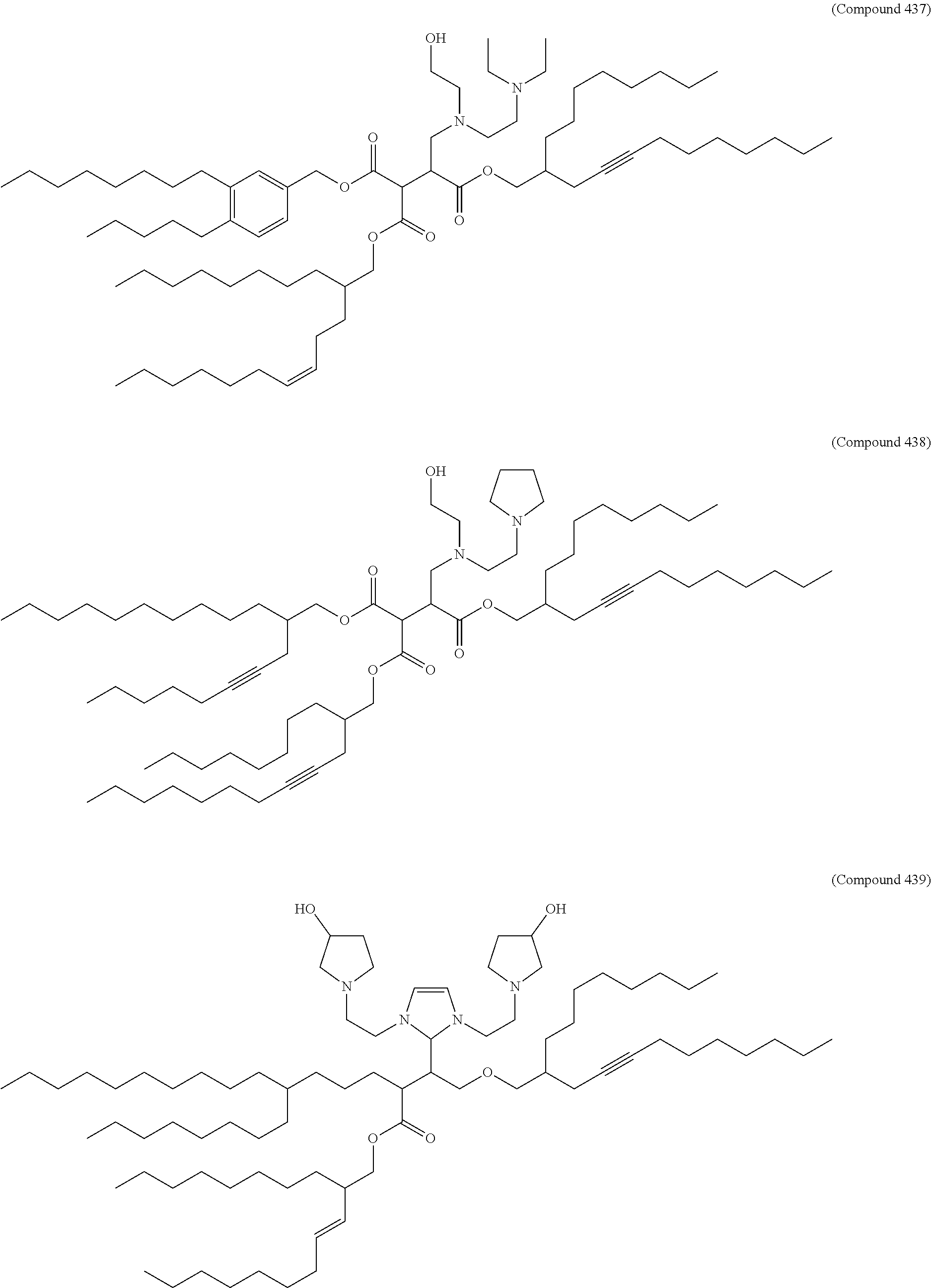

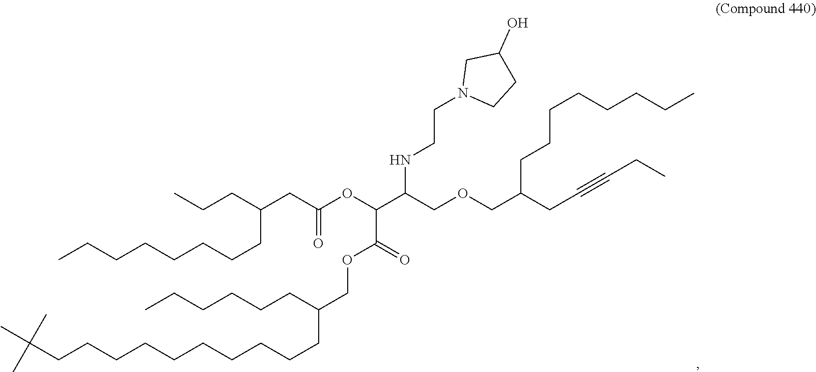 [SMILES: CCC#CCC(CCCCCCCC)COCC(NCCN1CCC(O)C1)C(OC(=O)CC(CCC)CCCCCCCC)C(=O)OCC(CCCCCC)CCCCCCCCCCC(C)(C)C]

or salts, or isomers thereof.

The amine moieties of the lipids according to Formula I, II, III, IV, V, VI, VII, VIII, IX, X, XI, XI, XII, XIII, or XIV may be protonated at physiological pH. The lipid may have a positive or partially positive charge at physiological pH. Such lipids may be referred to as ionizable lipids. Some lipids may also be zwitterionic (neutral molecules having both a positive and a negative charge).

As would be understood by a person of skill in the pertinent art, the present disclosure encompasses any and all reasonable combinations of any two or more embodiments described within each aspect of the disclosure, e.g., the compounds according to Formula I, II, III, IV, V, VI, VII, VIII, IX, X, XI, XI, XII, XIII, or XIV, including isomers, and salts, or pharmaceutical compositions thereof (below), as illustrated in certain claims.

In some embodiments, the ionizable lipid may have a pKₐ in the range of approximately 5.0 to approximately 8.0, more preferably, between approximately 5.5 and approximately 7.5, and even more preferably between approximately 6.0 and approximately 7.0. In some embodiments, the pKₐ may be between approximately 4.0 and approximately 9.5.

### II. Lipid Nanoparticle Compositions

Another aspect of the present disclosure provides a method of encapsulating and/or intercalating a biological and/or therapeutic agent within or on the surface of a lipid nanoparticle composition comprised of one or more ionizable lipids (e.g., amino lipid), helper lipids (e.g., neutral lipid), PEG conjugated or other modified lipids, and cholesterol with various pharmaceutically acceptable additives, such as, but not limited to, pH control agents (e.g., citric acid, sodium phosphate, sodium hydroxide, hydrochloric acid, acetic acid, tromethamine, histidine, succinic acid, and combinations thereof), isotonizing agents (e.g., sodium chloride, mannitol, sucrose, lactose, sorbitol), and antioxidants (e.g., α-tocopherol, ascorbic acid). Examples of biologically active and/or therapeutic agents include, but are not limited to: (1) polynucleotides such as mRNA, rRNA, RNAi, microRNA, plasmids, aptamers, DNA, cDNA; (2) antisense polynucleotides; (3) low molecular weight compounds (synthetic or naturally occurring) such as peptides, hormones, and antibiotics; and (4) proteins, etc.

In some embodiments, the biological and/or therapeutic agent is fully encapsulated within the lipid nanoparticle composition such that the biological and/or therapeutic agent is resistant to enzymatic degradation (e.g., by a nuclease). In certain embodiments, the biological and/or therapeutic agent may be partially encapsulated within the lipid nanoparticle composition such that the biological and/or therapeutic agent extends through the surface layer of the lipid nanoparticle composition, but is fully intercalated within a matrix of surface features, such as, but not limited to surface proteins, PEG or other polymer chains conjugated to a lipid such that the biological and/or therapeutic agent is fully resistant to enzymatic degradation (e.g., by a nuclease). In some preferred embodiments, the lipid nanoparticle compositions are non-toxic to mammals (e.g., humans).

In another aspect, this disclosure provides a method of treating a disease or disorder in a mammal (e.g., human) in need thereof. The method includes the step of administering to the mammal a therapeutically effective amount of a lipid nanoparticle composition comprising (a) a lipid component, including a phospholipid, a PEG conjugated lipid, a structural lipid, or a compound of Formula I, II, III, IV, V, VI, VII, VIII, IX, X, XI, XII, XIII, or XIV, and (b) a biological and/or therapeutic agent (e.g., mRNA).

In another aspect, this disclosure provides use of a lipid compound according to any of Formula I, II, III, IV, V, VI, VII, VIII, IX, X, XI, XII, XIII, or XIV, or a pharmaceutically acceptable salt thereof, in combination with a biological and/or therapeutic agent (e.g., mRNA) in the manufacture of a medicament for treating a disease or condition in a subject in need of treatment.

In some embodiments, the lipid nanoparticle composition may have a diameter of 1 m or smaller when measured by any means known in the art (e.g., dynamic light scattering (DLS), transmission electron microscopy, scanning electron microscopy, atomic force microscopy, or other methods). In some embodiments, the lipid nanoparticle compositions may have a diameter of 500 nm or smaller. In some preferred embodiments, the lipid nanoparticle compositions may have a diameter of 250 nm or smaller. In some embodiments, lipid nanoparticle compositions are vesicles comprising one or more lipid bilayers. In certain embodiments, a lipid nanoparticle composition comprises two or more concentric spherical, elliptical, or amorphous bilayers separated by aqueous compartments. Lipid bilayers may be functionalized by one or more ligands, proteins, and/or channels. Lipid bilayers may be cross-linked to one another.

In some embodiments, the lipid nanoparticle composition may comprise one or more ionizable lipids (e.g., amino lipids), neutral lipids, PEG- and other modified lipids (e.g., polyglycerol-modified, polyacrylamide-modified, polydimethylacrylamide-modified, polyvinylpyrrolidone-modified, hyaluronic acid-modified, heparin-modified, or polysialic acid-modified), or cholesterol. For example, the lipid nanoparticle composition may comprise at least one of the following ionizable lipids: Dlin-MC3-DMA DODMA, DODAP, SM-102, ALC-0315, C12-200, or one of the ionizable lipids described in Formula I, II, III, IV, V, VI, VII, VIII, IX, X, XI, XII, XIII, or XIV. In some embodiments, the lipid nanoparticle compositions comprise PEG₂₀₀₀-DMG or other PEG conjugated lipid. In some embodiments, the lipid nanoparticle compositions comprise cholesterol. In certain embodiments, the lipid nanoparticle composition comprises at least one neutral lipid (e.g., DSPE, DOPE, DSPC, HSPC, etc.).

In some embodiments, the lipid nanoparticle composition comprises (a) one or more active agent; (b) one or more ionizable lipid of Formula I, II, III, IV, V, VI, VII, VIII, IX, X, XI, XII, XIII, or XIV constituting from about 10 mol % to about 85 mol % of the total lipid present in the composition; (c) one or more neutral “helper” lipids constituting from about 5 mol % to about 40 mol % of the total lipid present in the composition; (d) one or more PEG-conjugated lipids that inhibit aggregation of particles constituting from about 0 mol % to about 10 mol % of the total lipid present in the composition; and (e) cholesterol constituting from about 10 mol % to 50 mol % of the total lipid present in the composition.

In some embodiments of the lipid nanoparticle composition, the phospholipid is selected from 1,2-dilinoleoyl-sn-glycero-3-phosphocoline (DLPC), 1,2-dimyristoyl-sn-glycero-phophocholine (DMPC), 1,2-dioleoyl-sn-glycero-3-phosphocholine (DOPC), 1,2-dipalmitoyl-sn-glycero-3-phophocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-dioleoyl-sn-glycero-3-phophoethanolamine (DOPE), palmitoyloleoyl-phosphatidylethanolamine (POPE), 1,2-diphytanoyl-sn-glycero-3-phosphoethanolamine, 1,2-dioleoyl-snglycero-3-phospho-rac-(1-glycerol) sodium salt (DOPG), sphingomyelin, and mixtures thereof.

In some embodiments of the lipid nanoparticle composition, the conjugated lipid that inhibits aggregation of nanoparticles comprises a polyethylene glycol-lipid conjugate (PEG-lipid), polyglycerol-lipid conjugates, polyoxazoline-lipid conjugates, polyvinylpyrrolidone-lipid conjugates, polyacrylamide-lipid conjugates, polydimethylacrylamide-lipid conjugates, hyaluronic acid-lipid conjugates, heparin-lipid conjugates, polysialic acid-lipid conjugates, or the like.

In some embodiments of the lipid nanoparticle composition, the PEG-lipid is selected from PEG-modified phosphatidylethanolamines, PEG-modified phosphatidic acids, PEG-modified ceramides, PEG-modified dialkylamines, PEG-modified diacylglycerols, PEG-modified dialkylglycerols, PEG-modified glycerides, PEG-modified sterols, and mixtures thereof.

In some embodiments of the lipid nanoparticle composition, the biological/therapeutic agent is a ribonucleic acid (RNA).

In some embodiments of the lipid nanoparticle composition, the RNA is selected from a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), self-amplifying mRNA (sa mRNA) and mixtures thereof.

In some embodiments of the lipid nanoparticle composition, the biological and/or therapeutic agent comprises a mRNA.

In some embodiments of the lipid nanoparticle composition, the mRNA comprises from about 300 to about 20,000 nucleotides.

In some embodiments of the lipid nanoparticle composition, the mRNA comprises at least one modified nucleotide.

In some embodiments of the lipid nanoparticle composition, the neutral phospholipid comprises distearoylphosphatidylcholine (DSPC).

In some embodiments of the lipid nanoparticle composition, the conjugated lipid that inhibits aggregation of particles comprises a polyethylene glycol-lipid conjugate (PEG-lipid).

In some embodiments of the lipid nanoparticle composition, the PEG-lipid conjugate comprises 1,2-dimyristoyl-rac-glycero-3-methoxypolyethylene glycol (DMG-PEG).

In some embodiments of the lipid nanoparticle composition, the PEG has an average molecular weight of 2000 Daltons.

In some embodiments of the lipid nanoparticle composition, the biological and/or therapeutic agent is an oligonucleotide.

In some embodiments of the lipid nanoparticle composition, the oligonucleotide comprises from about 10 to about 200 nucleotides.

In some embodiments of the lipid nanoparticle composition, the oligonucleotide comprises one or more modified nucleotides.

In some embodiments of the lipid nanoparticle composition, the oligonucleotide comprises at least one 2′-O-methyl (2′OMe) nucleotide.

In some embodiments, the lipid nanoparticle composition comprises of a biological and/or therapeutic agent, a compound according to Formula I, II, III, IV, V, VI, VII, VIII, IX, X, XI, XII, XIII, or XIV constituting from 10 mol % to 85 mol % of the total lipid present in the composition, a neutral “helper” phospholipid or derivative thereof, constituting from 5 mol % to 40 mol % of the total lipid in the composition, cholesterol, or a derivative thereof, constituting from 10 mol % to 50 mol % of the total lipid in the composition, a conjugated lipid that inhibits aggregation constituting from 0 mol % to 10 mol % of the total lipid in the composition. In some embodiments, the molar ratio of the ionizable nitrogen atoms in the compound according to Formula I, II, III, IV, V, VI, VII, VIII, IX, X, XI, XII, XIII, or XIV to the phosphate groups in the biological and/or therapeutic agent (N:P Ratio) is from 1 to 15.

In some embodiments, the lipid nanoparticle composition comprises of a biological and/or therapeutic agent, a compound according to Formula I, II, III, IV, V, VI, VII, VIII, IX, X, XI, XII, XIII, or XIV constituting from 20 mol % to 70 mol % of the total lipid present in the composition, a neutral “helper” phospholipid or derivative thereof, constituting from 5 mol % to 30 mol % of the total lipid in the composition, cholesterol, or a derivative thereof, constituting from 20 mol % to 50 mol % of the total lipid in the composition, a conjugated lipid that inhibits aggregation constituting from 0.25 mol % to 5 mol % of the total lipid in the composition. In some embodiments, the molar ratio of the ionizable nitrogen atoms in the compound according to Formula I, II, III, IV, V, VI, VII, VIII, IX, X, XI, XII, XIII, or XIV to the phosphate groups in the biological and/or therapeutic agent (N:P Ratio) is from 2 to 12.

In some embodiments, the, the lipid nanoparticle composition comprises of a biological and/or therapeutic agent, a compound according to Formula I, II, III, IV, V, VI, VII, VIII, IX, X, XI, XII, XIII, or XIV constituting from 40 mol % to 60 mol % of the total lipid present in the composition, a neutral “helper” phospholipid or derivative thereof, constituting from 5 mol % to 15 mol % of the total lipid in the composition, cholesterol, or a derivative thereof, constituting from 30 mol % to 45 mol % of the total lipid in the composition, a conjugated lipid that inhibits aggregation constituting from 0.5 mol % to 2 mol % of the total lipid in the composition. In some embodiments, the molar ratio of the ionizable nitrogen atoms in the compound according to Formula I, II, III, IV, V, VI, VII, VIII, IX, X, XI, XII, XIII, or XIV to the phosphate groups in the biological and/or therapeutic agent (N:P Ratio) is from 3 to 9.

A lipid nanoparticle composition may comprise one or more ionizable lipids in addition to a lipid described in Formula I, II, III, IV, V, VI, VII, VIII, IX, X, XI, XII, XIII, or XIV. Ionizable lipids may be selected from, but not limited to: 1,2-dioleyloxy-N,N-dimethylaminopropane (DODMA), 1,2-dioleoyl-3-dimethylammonium propane (DODAP), N-(4-carboxybenzyl)-N,N-dimethyl-2,3-bis(oleoyloxy)propn-1-aminium (DOBAQ), 1,2-dilinoleyloxy-N,N-dimethyl-3-aminopropane (Dlin-DMA), heptatriaconta-6,9,28,31-tetraen-19-yl 4-(dimehtylamino)butanoate (Dlin-MC3-DMA), 2-[2,2-bis[(9Z,12Z)-octadeca-9,12-dienyl]-1,3-dioxolan-4-yl]-N,N-dimethylethanamine (Dlin-K-DMA), 2,2-dilinoleyl-4-(2-dimethylaminoethyl)-[1,3]-dioxolane (Dlin-KC2-DMA), 9-heptandecanyl 8-{(2-hydroxyethyl)[6-oxo-6-(undecyloxy)hexyl]amino}octanoate (SM-102), [(4-hydroxybutyl)azanediyl]di(hexane-6,1-diyl) bis(2-hexyldecanoate) (ALC-0315), 1,1-((2-(4-(2-((2-(bis(2-hydroxydodecyl)amino)ethyl(2-hydroxydodecyl)amino) ethyl)piperazin-1-yl)ethyl)azanediyl)bis(dodecan-2-ol) (C12-200), 3,6-bis[4-[bis(2-hydroxydodecyl)amino]butyl]-2,5-piperazinedione (cKK-E12), and 1,1,4,10,10-pentakis(N-dodecylpropanamide)-1,4,7,10-tetraazadecane (98N12-5), and others.

A lipid nanoparticle composition may comprise one or more neutral “helper” lipids. Neutral lipids may be selected from, but not limited to: phospholipids such as lecithin, phosphatidylethanolamine, sphingomyelin, egg sphingomyelin (ESM), cephalin, cardiolipin, phosphatidic acid, cerebrosides, dicetylphosphates, 1,2-distearoyl-sn-glycero-3-phosphocholine (DSPC), 1,2-dimyristoyl-sn-glycero-phophocholine (DMPC), 1,2-dioleoyl-sn-glycero-3-phosphocholine (DOPC), 1,2-diundecanoyl-sn-glycero-phosphocholine (DUPC), 1,2-dipalmitoyl-sn-glycero-3-phosphocholine (DPPC), 1,2-dioleoyl-snglycero-3-phospho-rac-(1-glycerol) sodium salt (DOPG), 1,2-dipalmitoyl-sn-glycero-3-phospho-rac-(1-glycerol) sodium salt (DPPG), 1,2-dioleoyl-sn-glycero-3-phosphoethanolamine (DOPE), 1-palmitoyl-2-oleoyl-sn-glycero-3-phosphocholine (POPC), 1-palmitoyl-2-oleoyl-sn-glycero-3-phosphoethanolamine (POPE), 1-palmitoyl-2-oleyol-sn-glycero-3-phospho-rac-(1-glycerol) sodium salt (POPG), N-(3-malimide-1-oxopropyl)-1,2-dioleoyl-sn-glycero-3-phosphoethanolamine (DOPE-mal), 1,2-dipalmitoyl-sn-glycero-3-phosphoethanolamine (DPPE), 1,2,-dimyristoyl-sn-glycero-3-phosphoethanolamine (DMPE), 1,2-distearoyl-sn-glycero-3-phosphoethanolamine (DSPE), 1,2-dilinoleoyl-sn-glycero-3-phosphocholine (DLPC), 1,2-diphytanoyl-sn-glycero-3-phosphoethanolamine, monomethyl-phosphatidylethanolamine, dimethyl-phosphatidylethanolamine, 1,2-dierucoyl-sn-glycero-3-phosphoethanolamine (DEPE), 1-stearoyl-2-oleoyl-sn-glycero-3-phosphoethanolamine (SOPE), 1,2-dilinoleoyl-sn-glycero-3-phosphocholine (DLOPC), 1,2-dilauroyl-sn-glycero-3-phosphocholine (DLPC), and others and mixtures thereof. Other diacylphosphatidylcholine, diacylphosphatidylethanolamine, and diacylphosphatidylserine phospholipids may also be used. In some embodiments, the acyl groups in these lipids are acyl groups derived from fatty acids having C₁₀-C₂₄ carbon chains (e.g., lauroyl, myristoyl, palmitoyl, stearoyl, or oleoyl).

A lipid nanoparticle composition may also comprise one or more sterols. Sterols may be selected from the non-limiting list of cholesterol, cholesterol derivatives, ergosterol, and ergosterol derivatives. Non-limiting examples of cholesterol derivatives include 5α-cholestanol, 5α-coprostanol, cholesteryl-(2′-hydroxy)ethyl ether, cholesteryl-(4′-hydroxy)butyl ether, 6-ketocholestanol, thiocholesterol, cholesteryl acetate, cholesteryl sulfate, cholestane-3,5-diene, 5α-coprostane, cholestenone, 5α-cholestanone, cholesteryl dodecanoate, and others and mixtures thereof.

A lipid nanoparticle composition may also comprise one or more PEG conjugated or another polymer conjugated (e.g., polyglycerol-modified, polyacrylamide-modified, polydimethylacrylamide-modified, polyvinylpyrrolidone-modified, hyaluronic acid-modified, heparin-modified, polysialic acid-modified, etc.). PEG conjugated lipids may be selected from the following non-limiting list of 1,2-distearoyl-sn-glycero-3-phosphoethanolamine-N—[methoxy(polyethylene glycol)-2000] sodium salt (PEG2000-DSPE), 1,2-dipalmitoyl-sn-glycero-3-phosphoethanolamine-N-[methoxy(polyethylene glycol)-2000] sodium salt (PEG2000-DPPE), 1,2-dimyristoyl-rac-glycero-3-methoxypolyethylene glycol-2000 (PEG2000-DMG), distearoyl-rac-glycerol-PEG2000 (PEG2000-DSG), methoxypolyethyleneglycoloxy(2000)-N,N-ditetradecylacetamide (ALC-0159), 1,2-dioleoyl-sn-glycero-3-phosphoethanolamine-N-[amino(polyethylene glycol)-2000] sodium salt (DOPE-PEG1000-amine), 1,2-dioleoyl-sn-glycero-3-phosphoethanolamine-N-[amino(polyethylene glycol)-2000] sodium salt (DOPE-PEG2000-amine), 1,2-dioleoyl-sn-glycero-3-phosphoethanolamine-N-[carboxy(polyethylene glycol)-1000] sodium salt (DOPE-PEG1000-COOH), 1,2-dioleoyl-sn-glycero-3-phosphoethanolamine-N-[carboxy(polyethylene glycol)-2000] sodium salt (DOPE-PEG2000-COOH), and cholesterol-(polyethylene glycol-600) (PEG600-Chol), PEGylated ceramides, PEGylated phosphatidic acids, PEGylated phosphatidylethanolamines, PEGylated dialkylamines, PEGylated diacylglycerols, PEGylated dialkylglycerols, PEGylated glycerides, PEGylated sterols, and others and mixtures thereof. In some embodiments, the polyethylene glycol chain will have an average molecular mass of 2000 atomic mass units.

A lipid nanoparticle composition may comprise of additional components such as bilayer stabilizing components (e.g., polyamide oligomers [see e.g., U.S. Pat. No. 6,320,017]), peptides, proteins, detergents, lipid and ceramide derivatives (see e.g., U.S. Pat. No. 5,885,613).

In some embodiments of the present disclosure, the lipid component comprises an ionizable lipid, neutral lipid, a sterol, and a PEG conjugated lipid. In particular embodiments, the lipid component composition comprises one or more ionizable lipids, a neutral lipid, a sterol, a PEG conjugated lipid, and an antioxidizing excipient (e.g., α-tocopherol, N-acetylcysteine, ascorbic acid).

In some embodiments, the biological and/or therapeutic agent encapsulated by the lipid nanoparticle composition is a nucleic acid. In some embodiments the nucleic acid is an RNA or oligonucleotide that is fully or partially encapsulated within the lipid nanoparticle composition. Oligonucleotides may contain up to about 200 nucleotides and can be deoxyribooligonucleotides or ribooligonucleotides. A deoxyribooligonucleotide consists of a 5-carbon sugar called deoxyribose joined covalently to phosphate at the 5′ and 3′ carbons of this sugar to form an alternating, unbranched polymer. A ribonucleotide consists of a similar repeating structure where the 5-carbon sugar is ribose.

In some embodiments, the biological and/or nucleic acid is selected from fomivirsen, mipomersen, nusinersen, eteplirsen, inotersen, golodirsen, milasen, casimersen, patisiran, givosiran, lumasiran, inclisiran, pegaptanib, defibrotide, tozinameran, elsomeran, defibrotide, viltolarsen, casimersen, volanesorsen, Cas9 mRNA with or without its guide RNA, EPO mRNA, and the like, and combinations thereof.

The RNA of the present disclosure may be of various lengths, generally dependent on the form of the particular RNA form (e.g., mRNA, siRNA, dsRNA, RNAi, microRNA, etc.). For example, in particular embodiments, mRNA may from about 500 to about 100,000 nucleotide residues in length, while oligonucleotides may range from about 10 to about 200 nucleotides in length.

In some embodiments, the lipid nanoparticle composition may comprise one or more buffers. Other components may be added to enhance or maintain chemical stability, including but not limited to preservatives, surfactants, dispersants, and/or gases. In some embodiments, the pH of the lipid nanoparticle compositions may be from about pH 4.5 to 9.0. In some embodiments, the pH of the lipid nanoparticle compositions may be from about pH 5.0 to 8.5. In some preferred embodiments, the pH may be from about pH 5.5 to 8.0. In some more preferred embodiments, the pH may be from about pH 6.0 to 7.5.

In some embodiments, the lipid nanoparticle composition is produced via mixing of an alcoholic lipid solution (e.g., lipids dissolved in ethanol or isopropanol) with an aqueous solution consisting of the biological and/or therapeutic agents and a buffering agent. In some embodiments, the lipid nanoparticle composition is produced through a microfluidic device featuring chaotic mixing features. In some preferred embodiments, the lipid nanoparticle composition is produced through the mixing an alcoholic lipid solution with an aqueous solution consisting of the biological and/or therapeutic agents and a buffering agent in an impingement jet mixing device.

In some embodiments, the flow rate ratio of the aqueous solution (e.g., biological and/or therapeutic agent dissolved in a buffer solution) and alcoholic lipid solution (e.g., lipids dissolved in ethanol or isopropanol) is from about 12 to 1 to about 1 to 1. In some embodiments, the flow rate ratio of the aqueous solution (e.g., biological and/or therapeutic agent dissolved in a buffer solution) and alcoholic lipid solution (e.g., lipids dissolved in ethanol or isopropanol) is from about 6 to 1 to about 1 to 1. In some preferred embodiments, the flow rate ratio of the aqueous solution (e.g., biological and/or therapeutic agent dissolved in a buffer solution) and alcoholic lipid solution (e.g., lipids dissolved in ethanol or isopropanol) is from about 4 to 1 to about 1 to 1.

In some embodiments, the aqueous solution used in formation of the lipid nanoparticle composition is removed or diluted to a negligible amount by methods well known in the art (e.g., simple dilution, dialysis, ultracentrifugation, etc.). In some embodiments, the solution used to replace the removed aqueous solution may consist of one or more tonicity modifiers (e.g., sodium chloride, potassium chloride, mannitol, or dextrose), buffering agents, or cryoprotecting agents (e.g., sucrose, trehalose, mannitol, glycerol, etc.).

In some embodiments, the lipid nanoparticle composition may be stored in an acceptable pharmaceutically relevant carrier (e.g., a buffer or other solution designed to facilitate stability during storage or shipment). In some embodiments, the lipid nanoparticle composition may be refrigerated (e.g., being stored at a temperature of about 2° C. to about 8° C.). In other embodiments, the lipid nanoparticle composition may be stored in a carrier consisting of a buffering agent and a cryoprotectant, such as, but not limited to, sucrose, trehalose, or mannitol. In some embodiments, the lipid nanoparticle composition may be frozen (e.g., temperatures below 0° C. (e.g., about −5° C., −10° C., −15° C., −20° C., −30° C., −40° C., −50° C., −60° C., −70° C., −80° C., −90° C., −100° C.-120° C., −140° C., or −160° C.)). In still other embodiments, the lipid nanoparticle composition may be lyophilized in the presence of sucrose, lactose, or other saccharides or excipients (e.g., bulking agents, collapse temperature modifiers, amino acids, polyols, buffering agents, complexing agents, tonicity modifiers, or antioxidants). The lyophilized lipid nanoparticle composition cake can be stored preferably in a sterile lyophilization vial and later rehydrated with sterile water for injection.

### III. Application and Administration

The term “subject” and “patient” used herein refer to any animal (e.g., a mammal), including, but not limited to, humans, rodents, dogs, cats, horses, sheep, pigs, non-human primates, such as monkeys, and the like, to which the lipid nanoparticle compositions are administered.

The lipid nanoparticle compositions and methods of this disclosure provide for the delivery of a biological and/or therapeutic agent to treat a number of disorders. The lipid nanoparticle compositions of the present disclosure are suitable for the treatment of diseases or disorders relating to the deficiency or dysfunction of proteins and/or enzymes that are excreted or secreted by a cell into the surrounding extracellular fluid (e.g., clotting factors, components of the complement pathway cytokines, chemokines, chemoattractants, protein hormones, protein components of serum, antibodies, secretable toll-like receptors, and others). In some embodiments, the disease or disorder may involve a protein deficiency or misfolding (e.g., Alzheimer's disease, Parkinson's disease, cystic fibrosis, or Fabry disease). In other embodiments, the disease or disorder may not be caused by defect or deficient protein but would benefit from providing a secreted protein (e.g., spinal muscular atrophy, or leber congenital amaurosis). Diseases or disorders for which the present disclosure may be useful include, but are not limited to, Alzheimer's disease, Parkinson's disease, cystic fibrosis, Fabry disease, SMN1-related spinal muscular atrophy, Huntington's disease, muscular dystrophies (such as Dunchenne and Becker), human immunodeficiency virus (HIV), influenza, heart disease, cancers (such as e.g. breast, prostate, colorectal, renal, bladder, lymphomas, thyroid, endometrial, pancreatic), tuberculosis, multiple sclerosis, transthyretin amyloidosis, hemophilia diseases (such as, e.g., hemophilia B, hemophilia A), amyotrophic lateral sclerosis, GALT-related galcosemia, VEGF-related heart failure, propionic acidemia, ornithine transcarbamylase deficiency, Zika virus, rabies, SARS-CoV-2, malaria, tuberculosis, Hepatitis B, Gaucher's disease, Creutzfeldt-Jakob disease, nephrogenic diabetes insipidus, spinocerebellar ataxia, Dentatorubral-pallidoluysian atrophy, Sickle cell anemia, Machado-Joseph atrophy, retinitis pigmentosa, α-Antitrypsin deficiency, galactocerebrosidase deficiencies, Bardet-Biedel syndrome, Charlevoix-Daguenay, ethylmalonic aciduria, familial hypercholesterilemia, leprechaunism, Marfan syndrome, McKusick-Kaufman syndrome, Osteogenesis imperfecta, phenylketonuria, Tay-Sachs disease, cataracts, familial amyloidosis, Wilson's disease, Santavuori-Haltia disease, Jansky-Bielschowsky disease, Juvenile Batten disease, Juvenile Neuronal Ceroid Lipofuscinosis, and Pelizaeus-Merzbacher disease.

The lipid nanoparticle compositions may be administered to a patient. In some embodiments, the lipid nanoparticle composition comprises one or more additional biological and/or therapeutic agents, carriers, buffers, tonicity modifiers, cryoprotectants, or other suitable excipients in order to produce two or more distinct proteins or enzymes. In some embodiments, delivery of multiple biological and/or therapeutic agents (e.g., mRNA) may be utilized to treat diseases or disorders where the defective or missing protein is made up of subunits that are encoded by more than one gene. In some embodiments, the biological and/or therapeutic agent (e.g., mRNA) may be engineered in a manner that a single mRNA strand may encode for more than one subunit of the target protein.

### Definitions

Unless defined otherwise, all terms of art, notations and other scientific terms or terminology used herein have the same meaning as is commonly understood by one of ordinary skill in the art to which this disclosure belongs. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art. As appropriate, procedures involving the use of commercially available kits and reagents are generally carried out in accordance with manufacturer defined protocols and/or parameters unless otherwise noted. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, the singular forms “a,” “an,” and “the” include plural reference, and vice versa, any plural forms include singular reference, unless the context clearly dictates otherwise.

The term “about” or “approximately” used herein, unless otherwise defined, generally includes up to plus or minus 10% of the indicated number. For example, “about 10%” may indicate a range of 9% to 11%, and “about 30” may mean from 27 to 33. Sometimes preferably, “about” includes up to plus or minus 5% of the indicated value. When “about” is used before a range, it is applicable to both the lower end and upper end of the range.

The term “substantially” as used herein means “for the most part” or “essentially,” as would be understood by a person of ordinary skill in the art, and if measurable quantitatively, refers to at least 90%, preferably at least 95%, more preferably at least 98%.

The terms “comprising,” “having,” “including,” and “containing,” or the like, are to be construed as open-ended terms (i.e., meaning “including, but not limited to,”) unless otherwise noted.

The term “compound” used herein is meant to include all isomers and isotopes of the structure depicted. “Isotopes” refers to atoms having the same atomic number but different mass numbers, as a result of differing amounts of neutrons in the nuclei. For example, isotopes of hydrogen include deuterium and tritium. A compound, salt, or complex of the present disclosure can be prepared with solvent or water molecules to form solvates and hydrates by routine methods.

The term “isomer” used herein means any geometric isomer, tautomer, zwitterion, stereoisomer, enantiomer, or diastereomer of a compound, where applicable. Compounds may include one or more chiral centers (with an absolute configuration R or S, whether designated or not) and/or double bonds and may thus exist as stereoisomers, such as double-bond isomers (i.e., geometric E/Z isomers) or diastereomers (e.g., enantiomers (i.e., (+) or (−)) or cis/trans isomers). The present disclosure encompasses any and all isomers of the compounds described herein, including stereomerically pure forms (e.g., geometrically pure, enantiomerically pure, or diastereomerically pure) and enantiomeric and stereoisomeric mixtures (e.g., racemates). The means of identifying and resolving enantiomeric and stereometric mixtures of compounds into their component enantiomers or stereoisomer is well-known in the art.

The terms “alkyl” and “alkyl group” used herein refers to a linear or branched, fully saturated hydrocarbon having one or more, preferably 1 to 28, sometimes preferably 1 to 22, sometimes preferably 1 to 20, sometimes preferably 1 to 18, sometimes preferably 1 to 16, sometimes preferably 1 to 14, sometimes preferably 1 to 12, sometimes preferably 12 to 22, sometimes preferably 12 to 20, sometimes preferably 12 to 18, sometimes preferably 12 to 16, sometimes preferably 1 to 8, sometimes preferably 1 to 6, sometimes preferably 1 to 4, carbon atoms. The term “lower alkyl” or the like refers to C₁₋₆ alkyl, sometimes preferably C₁₋₄ alkyl, and sometimes more preferably methyl or ethyl. An alkyl group described herein may be optionally substituted.

The terms “alkenyl” and “alkenyl group” used herein refers to a linear or branched hydrocarbon having two or more, preferably 2 to 28, sometimes preferably 2 to 22, sometimes preferably 2 to 20, sometimes preferably 2 to 18, sometimes preferably 2 to 16, sometimes preferably 2 to 14, sometimes preferably 2 to 12, sometimes preferably 12 to 22, sometimes preferably 12 to 20, sometimes preferably 12 to 18, sometimes preferably 12 to 16, sometimes preferably 2 to 8, sometimes preferably 2 to 6, sometimes preferably 2 to 4, carbon atoms and at least one double bond. An alkenyl group may include one or more carbon-carbon double bonds. An alkenyl group described herein may be optionally substituted.

The terms “alkynyl” and “alkynyl group” used herein refers to a linear or branched hydrocarbon having two or more, preferably 2 to 28, sometimes preferably 2 to 22, sometimes preferably 2 to 20, sometimes preferably 2 to 18, sometimes preferably 2 to 16, sometimes preferably 2 to 14, sometimes preferably 2 to 12, sometimes preferably 12 to 22, sometimes preferably 12 to 20, sometimes preferably 12 to 18, sometimes preferably 12 to 16, sometimes preferably 2 to 8, sometimes preferably 2 to 6, sometimes preferably 2 to 4, carbon atoms and at least one carbon-carbon triple bond. An alkynyl group described herein may be optionally substituted.

The terms “aryl” and “aryl group” used herein refers to a C₆ to C₁₄ mono- or polycyclic (e.g., bicyclic or tricyclic) aromatic group. Representative aryl rings include, but are not limited to, phenyl, naphthyl, anthryl, phenanthryl, and biphenyl groups, sometimes preferably phenyl or naphthyl, and sometimes more preferably phenyl.

The term “heteroaryl,” “heteroaryl group,” or the like, used herein refers to an aromatic mono- or polycyclic (e.g., bicyclic or tricyclic) moiety of 5- to 14-membered ring atoms in which one or more, preferably one, two, or three, of the ring atom(s) is(are) selected from nitrogen, oxygen, or sulfur, the remaining ring atoms being carbon. Representative heteroaryl rings include, but are not limited to, pyrrolyl, furanyl, thienyl, oxazolyl isoxazolyl, thiazolyl, imidazolyl, triazolyl, tetrazolyl, pyridinyl, pyrimidinyl, pyrazinyl, pyridazinyl, indolyl, benzofuranyl, benzothiophenyl, thiophenyl, benzimidazolyl, quinolinyl, isoquinolinyl, quinazolinyl, quinoxalinyl, pyrazolyl, and the like.

The term “heteroalkyl,” or the like, used herein refers to alkyl radicals in which one or more skeletal chain atoms is a heteroatom independently selected from N, O, S, or combinations thereof. The heteroatom(s) are placed at any interior position of the heteroalkyl group or at the position at which the heteroalkyl group is attached to the remainder of the molecule. In some embodiments, up to two heteroatoms are consecutive. A heteroalkyl group described herein may be optionally substituted.

The term “carbocycle,” “carbocyclyl,” or “carbocyclic group” used herein refers to a non-aromatic mono- or multi-cyclic group having one or more, sometimes preferably one to two, sometimes more preferably one, ring(s) of carbon atoms. Rings may range in size from 3- to 18-carbon atom members, sometimes preferably 3- to 10-carbon atoms in the ring(s), and sometimes more preferably 3- to 8-carbon atoms in the ring(s). Carbocycles may also include one or more, sometimes preferably one or two, sometimes more preferably one, carbon-carbon double or triple bond.

The term “cycloalkyl” or “cyclic alkyl,” or the like, used herein refers to a saturated mono-carbocycle, preferably containing three to eight carbons (C₃₋₈) in the ring, and sometimes more preferably three to six carbons (C₃₋₆) in the ring. Carbocycles and cycloalkyls may be unsubstituted and substituted. Thus, cycloalkyl is a special subset of carbocyclic groups, often a more preferred subgroup.

The term “heterocycle,” “heterocyclyl,” “heterocyclic group,” or the like, used herein refers to a mono- or multi-cyclic group having one or more, preferably one to three, sometimes more preferably one or two, sometimes more preferably one, ring(s) of carbon atoms containing one or more, sometimes preferably one to three, sometimes more preferably one or two, heteroatom(s) independently selected from N, O, and S, wherein at least one of the rings containing a heteroatom is non-aromatic. Rings may range in size from 3- to 18-members, sometimes preferably 3- to 10-members in the ring(s), and sometimes more preferably 3- to 8-members in the ring(s). Heterocycles may also include one or more carbon-carbon, carbon-heteroatom, and/or heteroatom-heteroatom double or triple bonds. Heterocycles described herein may be optionally substituted.

The terms “alkylene,” “alkenylene,” “alkynylene,” “heteroalkylene,” “carbocyclylene,” “cycloalkylene,” and “heterocyclylene” used herein refer to the divalent linking groups of the parent prefix (e.g., alkyl, alkenyl, alkynyl, heteroalkyl, carbocyclyl, cycloalkylene, and heterocyclyl, respectively). For example, alkylene is the divalent moiety of alkyl and heterocyclylene is the divalent moiety of heterocyclyl. Sometimes, the suffix “ene” may be omitted for simplicity or convenience, for example, “alkylene” may be called “alkyl”, “alkenylene” may be called “alkenyl”, and “heteroalkylene” may be called “heteroalkyl” and “carbocyclylene” may be called “carbocyclyl”, and “heterocyclylene” may be called “heterocyclyl”, and so on. Sometimes, for convenience or simplicity, “alkenyl” or “alkenylene” may be simply called “alkene”, “alkynyl” or “alkynylene” may be simply called “alkyne”, “carbocyclyl” or “carbocyclylene” may be simply called “carbocyclic” or “carbocycle”, “cycloalkyl” or “cycloalkylene” may be simply called “cycloalkyl,” and “heterocyclyl” or “heterocyclylene” may be simply called “heterocycle” or “heterocyclic”, and so on. A person of ordinary in the art should readily be able to tell the exact meaning and structure of the group in the specific structural environments and context. Thus, such nomenclatures should not be viewed as ambiguous or treated as defects of disclosure. If deemed necessary and context justifies, renaming such groups in more proper terminology should not be treated as adding new matter.

The term “biodegradable group” used herein refers to a functional group that may facilitate metabolism of a lipid in an animal model (i.e., human). A biodegradable group may be selected from a group consisting of, but not limited to, —CO—, —CS—, —CO—O—, —O—CO—, —CS—O—, —CO—S—, —CS—S—, —O—CS—, —S—CO—, —S—CS—, and —S—S—.

Alkyl, alkenyl, alkynyl, carbocyclic, and heterocyclic groups may be optionally substituted unless otherwise specified or noted. Optional substituents may be selected from, but are not limited to, a halogen (e.g., chloride, bromide, fluoride, iodide group), a carboxylic acid, a carbonyl, a carbonate, an alkoxy, an acetal, a phosphate, a thiol, a disulfide, a sulfoxide, a sulfinic acid, a sulfonic acid, a thioaldehyde, a sulfate, a sulfonyl, an amide, an azido, a nitro, a cyano, an isocyano, an acyloxy, an amino, a carbamoyl, a sulfonamide, an alkyl, an alkenyl, an alkynyl, a carbocycle, or a heterocycle group. In some embodiments, the substituted groups may also be further substituted with one or more substituents as defined herein.

The phrases “substituted or unsubstituted” or the like and “optionally substituted” or the like may be used interchangeably in the disclosure. When any group in any compound or structure is indicated to be either “substituted” and/or “unsubstituted”, it means that the group can be optionally substituted by one or more, preferably one to five, and sometimes more preferably one to three, substituents independently selected from halogen, cyano, nitro, amino, alkyl, haloalkyl, alkoxy, haloalkoxy, aryl, alkylthio, alkylamino, alkylsulfonyl (alkylsulfone), alkylsulfoxyl (alkylsulfoxide), acyloxy, carboxylic acid, carboxylic ester, and carboxamide groups, or the like. The alkyl groups can be 1-10 carbon atoms, sometimes preferably 1-6 carbon atoms, sometimes more preferably 1-4 carbon atoms. The esters can be the esters of C₁ to C₁₀ alcohols, sometimes preferably C₁ to C₆ alcohols, sometimes more preferably C₁ to C₄ alcohols. An expression such as “optionally substituted alkylene, alkenylene, or alkynylene” should be interpreted to mean that each of the alkylene, alkenylene, and alkynylene is optionally substituted.

In some embodiments, when an alkyl, alkenyl, alkynyl, cycloalkyl, heterocyclyl, aryl, heteroaryl group, or the like, or a moiety thereof, is substituted, the substituent group(s) can be substituted at any available connection point(s), and the substituents can be one or more, sometimes preferably 1 to 5, and sometimes more preferably 1 to 3, group(s) independently selected from C₁-C₆ alkyl, halogen, C₁-C₆ alkoxy, C₁-C₆ alkenyl, C₁-C₆ alkynyl, C₁-C₆ alkylthio, C₁-C₆ alkylamino, di-(C₁-C₆ alkyl)amino, thiol, hydroxyl, nitro, cyano, amino, C₃-C₆cycloalkyl, 5- to 10-membered heterocyclyl, C₆-C₁₀ aryl, 5- to 10-membered heteroaryl, C₃-C₆ cycloalkoxy, C₁-C₆ cycloalkylthio, 5- to 10-membered heterocyclylthio and oxo group.

In some embodiments, sometimes preferably, the substituents are independently selected from C₁-C₆ alkyl, halogen, C₁-C₆ alkoxy, C₁-C₆ alkylthio, C₁-C₆ alkylamino, di-(C₁-C₆ alkyl)amino, thiol, hydroxyl, nitro, cyano, amino, and oxo group. In some embodiments, sometimes more preferably, the substituents are independently selected from C₁-C₄ alkyl, halogen, C₁-C₄ alkoxy, C₁-C₄ alkylthio, C₁-C₄ alkylamino, di-(C₁-C₄ alkyl)amino, thiol, hydroxyl, nitro, cyano, and amino. As a person of ordinary skill in the art would understand, an oxo (═O) group cannot be a substituent of an aryl or heteroaryl group, or at an unsaturated carbon in any other group.

The term “linker” used herein refers to a moiety connecting two other moieties. A linker may include one or more groups, including but not limited to phosphate groups, alkylene groups, alkenylene groups, carbocyclic groups, heterocyclic groups, amidates, or glycerols.

The phrase “longest chain of atoms in the compound,” or the like, used herein refers to the greatest length (by counting of atom numbers) from the end (not including H's) of a substituent chain to the end (not including H's) of another substituent chain in the compound molecule, including any atoms most directly connecting the two substituent chains (i.e., the smallest number of intervening atoms between the two substituent chains). For example, the greatest length from the end of one substituent chain to the end of another substituent chain in Compound 1 is 19 atoms; the greatest length from the end of one substituent chain to the end of another substituent chain in Compound 128 is 40 atoms; and the greatest length from the end of one substituent chain to the end of another substituent chain in the Compound 206 is 52 atoms. While not intending to be bound by theory, in order to maintain the properties and features of the LNP compounds disclosed, a preferred range of “longest chain of atoms in the compound” is from about 18 to about 70 atoms. In some embodiments, the longest chain of atoms is between 18 and 60 atoms. In some embodiments, the longest chain of atoms, is between 18 and 50 atoms.

The term “lipid component” used herein is a component of a lipid nanoparticle composition that includes one or more lipids. For example, the lipid component may include one or more ionizable, PEG conjugated, structural, or other lipids (e.g., phospholipids).

The term “N:P ratio” used herein refers to the molar ratio of ionizable nitrogen atoms (in the physiological pH range) in a lipid to the phosphate groups in a nucleic acid (e.g., RNA).

The term “lipid nanoparticle composition” used herein refers to a composition comprising one or more lipids. Lipid nanoparticle compositions are typically sized in the order of micrometer or nanometer or smaller and may include a lipid bilayer. Lipid nanoparticle compositions encompasses particles such as lipid nanoparticles (LNPs), liposomes, lipoplexes, nano-emulsions, and polymeric nanoparticles. For example, a lipid nanoparticle composition may be a liposome with a diameter of 600 nm or less.

Lipid nanoparticle compositions may also comprise salts of one or more compounds. Salts may be pharmaceutically acceptable salts, sometimes preferably. The term “pharmaceutically acceptable salts” used herein refers to derivatives of the disclosed compounds wherein the parent compound is altered by converting an existing acid or base moiety to its salt form (e.g., by reacting a free base group with an organic acid). Examples of pharmaceutically acceptable salts include, but are not limited to, mineral or organic acid salts of basic residues, such as amines; alkali or organic salts of acidic residues, such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptanoate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate (also known as tosylate), undecanoate salts, and the like. Representative alkali or alkaline earth metal salts include sodium, lithium, potassium, calcium, magnesium, and the like as well as nontoxic ammonium, quaternary ammonium, and amine cations, including, but not limited to, ammonium tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains an acidic or basic moiety by conventional chemical methods, well-known in the art. Lists of suitable salts are found in Remington's Pharmaceutical Sciences, 17^{th} ed., Mack Publishing Company, Easton, Pa, 1985, p. 1418, Pharmaceutical Salts: Properties, Selection, and Use, P. H. Stahl and C. G. Wermuth (eds.), Wiley-VCH, 2008; Berge et al., Journal of Pharmaceutical Science, 66, 1-19 (1977); and in The Orange Book (Food & Drug Administration, Washington, D.C. on their website), each of which is incorporated herein by reference in its entirety.

One of ordinary skill in the art will appreciate that the synthetic methods described herein may utilize a variety of protecting groups. The term “protecting group” used herein refers to a particular functional moiety (e.g., O, S, N) that is temporarily blocked so that a reaction may be carried out selectively at another reactive site in a multifunctional compound. In some embodiments, a protecting group reacts selectively in good to excellent yields to provide a protected substrate that is stable to the reaction conditions of the subsequent reaction, yet easily and selectively removed in good to excellent yield by readily available, preferably non-toxic reagents that do not react with other functional groups. In some embodiments, a protecting group does not generate new stereogenic centers and has a little to no additional functionality to avoid further sites of reaction. Protecting groups may be used to form an easily separatable derivative. Oxygen, sulfur, nitrogen, and carbon protecting groups may be utilized as described herein. Protecting groups for a hydroxyl functional group may be selected from the following non-limiting list: methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (MBom), (4-methoxyphenoxy)methyl (p-AOM), guaiacolmethyl (GUM), t-butoxymethyl, 4-pentenyloxymethyl (POM), siloxymethyl, 2-methyoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahyxropyranyl, tetrahydrothiopyranyl, 1-methoxycyclohexyl, 4-methoxytetrahydropyranyl (MTHP), 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 2-bromoethyl, 2,2,2-trichloroethyl, 1-[2-(trimethylsilyl)ethoxy]ethyl, 2-trimethylsilylethyl, 4-methoxyphenacyl, t-butyl, cyclohexyl, allyl, p-methoxyphenyl, p-chlorophenyl, p-nitrophenyl, 2,4-dinitrophenyl, benzyl, p-methoxybenzyl, halobenzyls, 2-picolyl, 4-picolyl, 2-quinolynylmethyl, diphenylmethyl, bis(4-methoxyphenyl)methyl, triphenylmethyl, trimethylsilyl (TMS), triethylsilyl (TES), triisopropylsilyl (TIPS), dimethylisopropylsilyl (IPDMS), t-butyldiphenylsilyl (TBDPS), tribenzylsilyl, triphenylsilyl, diphenylmethylsilyl (DPMS), t-butylmethoxyphenylsilyl (TBMPS), formate, benzoylformate, acetate, haloacetates, pivalate, adamantoate, crotonate, benzoate, mesitoate, alkyl methyl carbonate, 9-fluorenylmethyl carbonate (Fmoc), alkyl 2,2,2-trichloroethyl carbonate (Troc), 2-(trimethylsilyl)ethyl carbonate (TMSEC), 2-(phenylsulfonyl)ethyl carbonate (Psec), 2-(triphenylphsphonio)ethyl carbonate (Peoc), methanesulfonate (mesylate), benzylsulfonate, and tosylate (Ts). Protecting groups for an amino functional group may be selected from the following non-limiting list: methyl carbamate, ethyl carbamate, 9-fluorenylmethyl carbamate (Fmoc), 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2,2-dibromoethyl carbamate (DB-t-BOC), 1-methyl-1-(4-biphenyl)ethyl carbamate (Bpoc), t-butyl carbamate (BOC), 1-adamantyl carbamate (Adoc), vinyl carbamate (Voc), allyl carbamate (Alloc), cinnamyl carbamate (Coc), 4-nitrocinnamyl carbamate (Noc), benzyl carbamate (Cbz), p-methoxybenzyl carbamate (Moz), 4-methylsulfinylbenzyl carbamate (Msz), [2-(1,3-dithianyl)]methyl carbamate (Dmoc), 4-methylthiophenyl carbamate (Mtpc), 2,4-dimethylthiophenyl carbamate (Bmpc), t-amyl carbamate, cyclobutyl carbamate, cyclohexyl carbamate, formamide, acetamide, chloroacetamide, trichloroacetamide, trifluoroacetamide, phenylacetamide, picolinamide, benzamide, acetoacetamide, N-[2-(trimethylsilyl)ethoxy]methylamine (SEM), quaternary ammonium salts, N-benzylamine, N-triphenylmethylamine (Tr), N-[(4-methoypehnyl)diphenylmethyl]amine (MMTr), N-ferrocenylmethylamino (Fcm), N-benzylideneamine, N-diphenlmethyleneamine, diphenylphosphinamide (Dpp), dimethylthiophosphinamide (Mpt), diphenylthiophosphinamide (Ppt), diphenyl phosphoramidate, benzenesulfenamide, o-nitrobenzenesulfenamide (Nps), p-toluenesulfonamide (Ts), benzenesulfonamide, methanesulfonamide (Ms), benzylsulfonamide, trifluoromethylsulfonamide, and phenacylsulfonamide. Additionally, a variety of protecting groups are described in Greene's Protective Groups in Organic Synthesis, Fifth Ed. Wuts, P. G. M., Ed., John Wiley & Sons, New Jersey: 2014, the entire contents of which are hereby incorporated by reference.

The term “independently selected” used herein refers to the selection of one or more R groups independently from other R groups within the same structure (e.g., R groups can be the same or different).

The term “pharmaceutically acceptable” used herein describes a material that is not biologically or otherwise undesirable, i.e., without causing an unacceptable level of undesired biological effects or interactions in a deleterious manner (e.g., toxicity, irritation, allergic response, or other problem or complication).

The term “polypeptide” used herein refers to a polymer of amino acid residues, typically joined by peptide bonds, that can be produced naturally or synthetically.

The term “biological agent” and “therapeutic agent” used herein refers to any agent that, when administered to a subject (e.g., cell, mouse, rat, human) has a therapeutic or diagnostic effect and/or elicits a desired pharmacological or biological effect. Biological and therapeutic agents also refer to “active agents”, which may include but are not limited to chemotherapeutic agents, small molecule drugs, nucleic acids, proteins, radioactive agents, and cytotoxins.

The term “nucleic acid” used herein refers to biopolymers and macromolecules comprised of nucleotides, such as RNA, DNA, or oligonucleotides.

The term “DNA” used herein refers to a deoxyribonucleic acid that may be naturally or non-naturally occurring. For example, a DNA may include modified and/or non-naturally occurring components, such as one or more nucleobases, nucleotides, or linkers. A DNA may have a nucleotide sequence encoding for an RNA sequence designed to produce a polypeptide of interest. For example, a DNA may encode for a messenger RNA (mRNA).

DNAs may be selected from, but not limited to, the group of plasmids, aptamers, complementary DNA (cDNA), and mixtures thereof.

The term “plasmid” used herein refers to a small extrachromosomal DNA molecule within a cell that is physically separated from the chromosomal DNA and may replicate independently.

The term “aptamer” used herein refers to a short, single-stranded DNA (ssDNA) or RNA (ssRNA) molecule that selectively binds to a specific target (e.g., protein, peptide, carbohydrate, small molecule, toxin, or living cell).

The term “RNA” used herein refers to a ribonucleic acid that may be naturally or non-naturally occurring. For example, an RNA may include modified and/or non-naturally occurring components, such as one or more nucleobases, nucleotides, or linkers. An RNA may include a cap structure, a chain terminating nucleoside, a stem loop, a poly-adenosine sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example in vivo translation of an mRNA inside a cell, may produce the encoded polypeptide. RNAs may be selected from, but not limited to, the group of small interfering RNA (siRNA), asymmetrical interfering RNA (aiRNA), microRNA (miRNA), dicer-substrate RNA (dsRNA), small hairpin RNA (shRNA), mRNA, ribosomal RNA (rRNA), aptamers, and mixtures thereof. Nucleic acids may contain any number of modifications including but not limited to the following: modified backbone structure (e.g., phosphorothioates, chiral phosphrothioates, phosphorodithioates, phosphotriesters, aminoalkylphosphotriesters, alkyl phosphonates, phosphinates, phosphoramidates, and boranophosphates), salt form modifications (e.g., sodium, potassium, ammonium, etc.), pseudouridine (V) replacement, methyl-6-adenosination (m⁶A), methyl-5-cytosination (m⁵C), ribose methylation (2′-OMe), and sugar identity (e.g., deoxyribose vs ribose). For more information on nucleic acids modifications see McKenzie, L. K., et al. Chemical Society Reviews, 50, 5126-5164 (2021), Ochoa, S., et al. Molecules, 25 (20) 4659 (2020), Prakash, T. P., et al. Journal of Medicinal Chemistry 48, 4247-4253 (2005), and Zhang, H. Y., et al. Current Topics in Medicinal Chemistry 6, 893-900 (2006) the entire contents of which are hereby incorporated by reference.

The term “polydispersity index” used herein is a ratio that describes the homogeneity of a particle size distribution in a system or sample. A small value, e.g., less than 0.2, indicates a narrow particle size distribution, while a large value, e.g., 0.8, indicates a broad particle size distribution.

The term “size” or “mean size” used herein in context of lipid nanoparticle compositions refers to the mean diameter of a lipid nanoparticle composition.

The term “zeta potential” used herein refers to the electrokinetic potential of a lipid or lipid nanoparticle composition.

The term “subject” or “patient” used herein refers to a human patient or a mammalian animal, such as cat, dog, cow, horse, monkey, or the like.

The term “contacting” used herein refers to an establishment of physical connection between two or more entities. For example, contacting a cell with a lipid nanoparticle composition means that the cell and nanoparticle are made to share a physical connection. Methods of contacting cells with external entities are well known in the arts.

The terms “delivering” and “delivery” used herein means providing an entity to a destination. For example, delivering a biological or therapeutic agent to a subject may involve administering a lipid nanoparticle composition comprising the biological or therapeutic agent to the subject by methods well known in the biological arts (e.g., intravenous, intradermal, subcutaneous, or intramuscular routes).

The term “encapsulation efficiency” used herein refers to the amount of a biological or therapeutic agent that becomes part of a lipid nanoparticle composition, relative to the initial total amount of biological or therapeutic agent used in the preparation of a lipid nanoparticle composition. For example, if 30 μg of biological or therapeutic agent are encapsulated within a lipid nanoparticle composition out of a total of 40 μg of the biological or therapeutic agent initially provided to the composition, the encapsulation efficiency may be given as 75%.

The term “encapsulation” used herein may refer to complete, substantial, or partial enclosure, confinement, surrounding, or encasement. Encapsulation efficiency may be determined by a RiboGreen® assay. Ribogreen® is an ultra-sensitive fluorescent nucleic acid stain for quantifying oligonucleotides and single stranded DNA or RNA in solution (available from Invitrogen Corporation, Waltham, Mass.).

The term “fully encapsulated” used herein indicates that the biological and/or therapeutic agent resides within the lipid nanoparticle composition in such a manner that it is not significantly degraded after exposure to serum or a nuclease assay that would significantly degrade the free biological and/or therapeutic agent. In some embodiments, less than 25% of the biological and/or therapeutic agent is degraded in a treatment that would normally degrade 100% of the free biological and/or therapeutic agent. In some preferred embodiments, less than 10% of the biological and/or therapeutic agent is degraded. In some more preferred embodiments, less than 5% of the biological and/or therapeutic agent is degraded. In still more preferred embodiments, less than 1% of the biological and/or therapeutic agent is degraded. Fully encapsulated also suggests that the particles are serum stable, that is, that they do not rapidly decompose into their component parts upon in vivo administration.

The term “expression” used herein refers to the translation of an mRNA or similar nucleic acid into a polypeptide or protein as well as post-translational modification of a polypeptide or protein.

The term “transfection” used herein refers to the introduction of a species (i.e., a biological or therapeutic agent, e.g., RNA) into a cell.

The term “enhanced delivery” used herein refers to delivery of more of a biological and/or therapeutic agent by a lipid nanoparticle composition to a tissue or cell compared to the level of delivery of a biological and/or therapeutic agent by a control lipid nanoparticle composition to the tissue or cell (e.g., by Dlin-MC3-DMA, SM-102, ALC-0315, or DODMA). The level of delivery may refer to at least 1.5-fold, at least 2-fold, at least 3-fold more, at least 4-fold more, at least 5-fold more, at least 10-fold more than the control lipid nanoparticle composition. The level of delivery of the biological and/or therapeutic agent to a particular tissue or cell of interest may be measured by comparing the amount of desired protein produced in a tissue or cell of interest to the amount of total protein in said tissue or cell. Enhanced delivery of a lipid nanoparticle formulation to a tissue or cell may be determined in a subject other than the one being treated (e.g., a rat, a pig, a dog, a non-human primate, etc.).

The following abbreviations are used:

| | |
|---|---|
| ACN: | acetonitrile |
| aiRNA: | asymmetrical interfering RNA |
| BCA Assay: | bicinchoninic acid assay |
| CDCl₃: | deuterated chloroform |
| cDNA: | complementary DNA |
| Chol: | cholesterol |
| DCM: | dichloromethane |
| DIEA: | N,N-diisopropylethylamine |
| DMAP: | 4-dimethylaminopyridine |
| DMPC: | 1,2-dimyristoyl-sn-glycero-3-phosphocholine |
| DNA: | deoxyribonucleic acid |
| DPPC: | 1,2-dipalmitoyl-sn-glycero-3-phosphocholine |
| DSPC: | 1,2-distearoyl-sn--glycero-3-phosphocholine |
| dsRNA: | dicer-substrate RNA |
| EDC: | 1-ethyl-3-(3-dimethylaminopropyl)carbodiimide |
| | hydrochloride |
| EtOH: | ethanol |
| HFIP: | hexafluoroisopropanol |
| HSPC: | L-α-phosphatidylcholine, hydrogenated |
| IPA: | Isopropanol |
| LNP: | lipid nanoparticle |
| mCPBA: | meta-chloroperoxybenzoic acid |
| MeOH: | methanol |
| miRNA: | microRNA |
| mRNA: | messenger RNA |
| O/N: | overnight |
| PC: | phosphatidylcholine |
| PEG: | polyethylene glycol |
| PEG-DMG: | 1,2-dimyristoyl-rac-glycero-3- |
| | methoxypolyethylene glycol-2000 |
| PG: | phosphatidylglycerol |
| RNA: | ribonucleic acid |
| RNAi: | RNA interference |
| rRNA: | ribosomal RNA |
| rt: | room temperature |
| shRNA: | small hairpin RNA |
| siRNA: | small interfering RNA |
| TEA: | triethylamine |
| TFA: | trifluoroacetic acid |
| THF: | tetrahydrofuran |
| TLC: | thin layer chromatography |

### EXAMPLES

The present disclosure will be described in greater detail by way of specific examples. The following examples are offered for illustrative purposes and are not intended to limit the disclosure in any manner. Those of skill in the art will readily recognize a variety of non-critical parameters which can be modified or changed to yield similar results.

It is to be understood that all above descriptions and below examples are intended to be illustrative and not restrictive. Many embodiments will be apparent of those skilled in the art upon reading the descriptions or examples. The scope of the disclosure should, therefore, be determined not with reference to the description or example but should instead be determined with reference to the appended claims, along with the full scope of equivalents to which such claims are entitled. The disclosures of all articles and references, including patent applications, patents, PCT publications, and GenBank Accession Nos., are incorporated herein by reference for all purposes.

### Example 1

### Materials and Methods

All commercial chemical reagents and solvents were purchased from Sigma Aldrich (St. Louis, MO), Fisher Scientific (Hampton, NH) and used without further purification. ¹H NMR spectra were recorded on an Agilent 500 MHz spectrometer at room temperature. Chemical shifts (δ) are given in parts per million and references to the residual solvent signal; and all coupling constants (J) are reported in Hz.

mRNA: All mRNA molecules used in these studies were purchased from TriLink BioTechnologies (San Diego, CA) without further modifications.

Lipid encapsulation of mRNA: In some embodiments, mRNA molecules were encapsulated into nucleic acid-lipid particles composed of one or more of the following lipids: Lipid conjugate PEG-DMG, ionizable lipid DODMA, DODAP, Dlin-MC3-DMA, SM-102, ALC-0315, DSPC, a compound from Formula I, II, III, IV, V, VI, VII, VIII, IX, X, XI, XII, XIII, or XIV, and/or cholesterol.

### Example 2

### Synthesis of Compounds According to Formula I

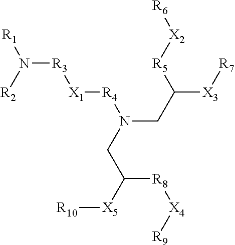

### Representative Procedure 1

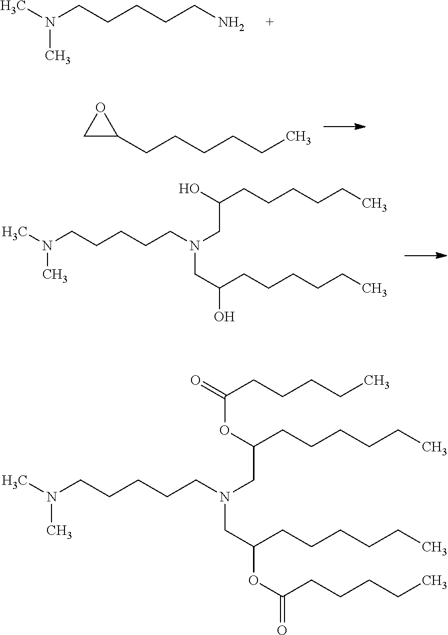

### 1,1′-{[5-dimethylamino)pentyl]azanediyl}di(octan-2-ol) (Method A)

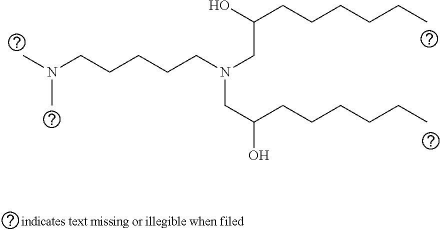 [SMILES: CCCCCCC(O)CN(CCCCCN(C)C)CC(O)CCCCCC]

To a dry flask charged with a stir bar was added 5-(dimethylamino)amylamine (143 μL, 1 mmol) and 1,2-epoxyoctane (306 μL, 2 mmol). The flask was sealed, and the mixture was stirred at 90° C. for 3 days. The reaction mixture was cooled to room temperature. The title compound was collected as a pale-yellow viscous oil and used without further purification.

### 1,1′-{[5-(dimethylamino)pentyl]azanediyl}di(octane-1,2-diyl) dihexanoate (Method B)

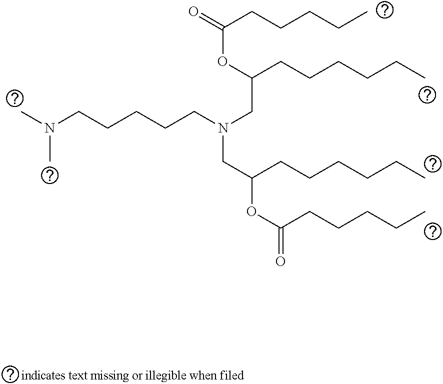 [SMILES: CCCCCCC(CN(CCCCCN(C)C)CC(CCCCCC)OC(=O)CCCCC)OC(=O)CCCCC]

To a solution of 1,1′-{[5-dimethylamino)pentyl]azanediyl}di(octan-2-ol) (193 mg, 0.5 mmol) and triethylamine (153.3 μL, 1.1 mmol) in dichloromethane (2 mL) was added hexanoyl chloride (232.2 μL, 1.5 mmol) dropwise. The reaction was allowed to stir at room temperature overnight. The reaction mixture was diluted with dichloromethane and washed with saturated sodium bicarbonate. The organic layer was separated, washed with brine, and dried over Na₂SO₄. The organic layer was filtered and evaporated in vacuo. The title compound was collected as a pale-yellow viscous oil and used without further purification.

### Example 3

### Synthesis of Compounds According to Formula II

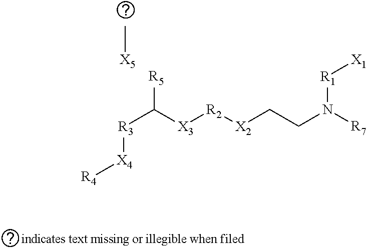

### Representative Procedure 2

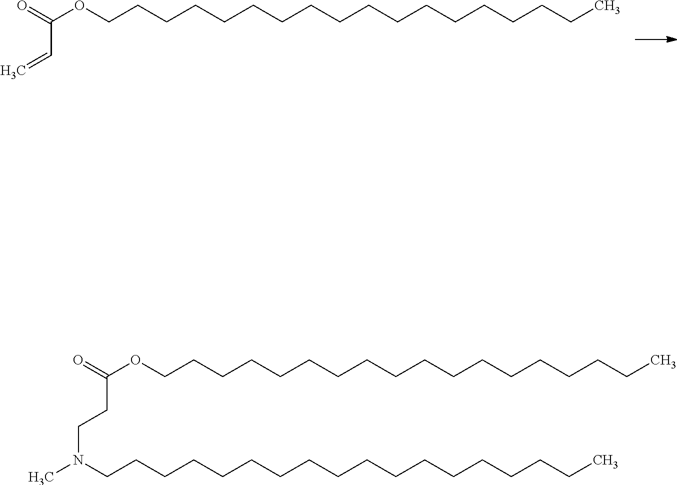

### octadecyl 3-[octadecyl(methyl)amino]propanoate (Method C)

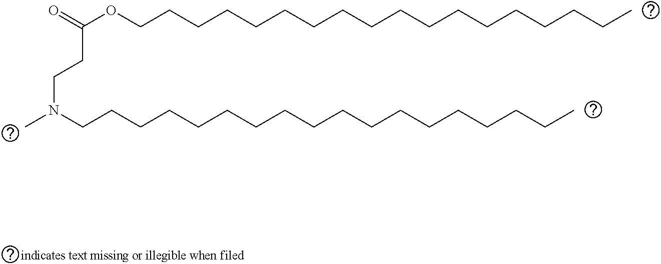 [SMILES: CCCCCCCCCCCCCCCCCCOC(=O)CCN(C)CCCCCCCCCCCCCCCCCC]

A vial was charged with octadodecanyl prop-2-enoate (162 mg, 0.5 mmol) and a stir bar was added. N-methyloctadecylamine (142 mg, 0.5 mmol) and 1.0 mL of isopropanol/hexafluoroisopropanol (v:v=3:1) were added and the vial was sealed. The mixture was allowed to stir at 50° C. for 3 hours. The reaction mixture was concentrated under vacuum and the crude product was used without further purification.

### Synthesis of Intermediates

### Fatty Acrylates: Oleyl Acrylate

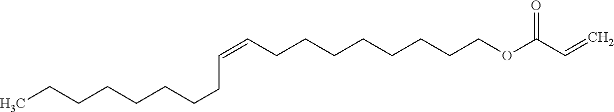 [SMILES: C=CC(=O)OCCCCCCCC/C=C\CCCCCCCC]

Oleyl alcohol (134 mg, 5 mmol) and triethylamine (55 mg, 5.5 mmol) was mixed in 1.0 mL dry dichloromethane in a glass vial. Acryloyl chloride (46 mg, 0.5 mmol) was slowly added into the resulting mixture. The vial was capped, and the mixture was stirred at room temperature for 3 hours. The reaction mixture was concentrated under vacuum. The crude product obtained was used directly without further purification.

### Example 4

### Synthesis of Compounds According to Formula III

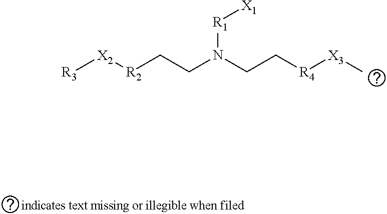

### Representative Procedure 3

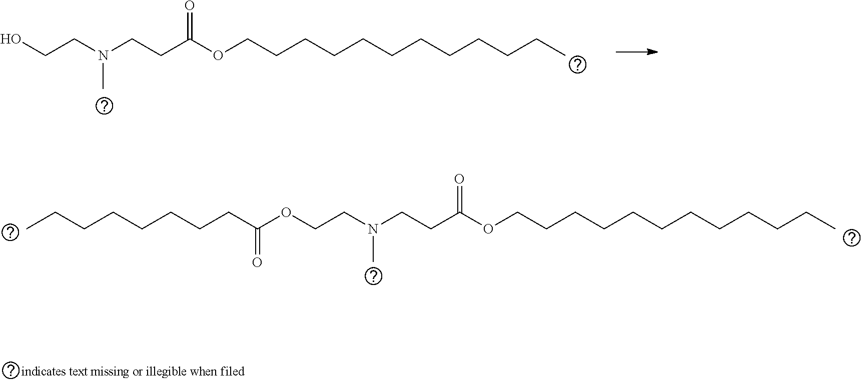

### 2-{methyl[3-oxo-3-(dodecoxy)propyl]amino}ethyl nonanoate (Method D)

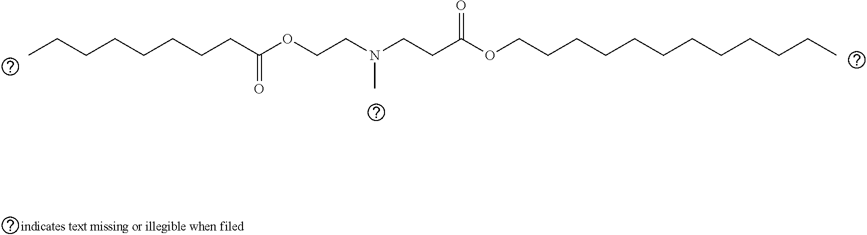 [SMILES: CCCCCCCCCCCCOC(=O)CCN(C)CCOC(=O)CCCCCCCC]

A glass vial was charged with dodecanyl 3-[(2-hydroxyethyl)(methyl)amino]propanoate (315 mg, 1.0 mmol) and triethylamine (110 mg, 1.1 mmol). Anhydrous dichloromethane (1 mL) was added to the mixture. Nonanoyl chloride was dissolved in anhydrous dichloromethane (1 mL) and added to the mixture dropwise. The solution was stirred for 2 hours at room temperature, then the solvent was removed by vacuum. The product was then treated with hexanes (5 mL) and mixed well to precipitate white, needle-like crystals. The mixture was filtered and the hexane was removed under vacuum to yield the title compound as a pale-purple, viscous liquid and used without further purification.

### Synthesis of Intermediates

### Dodecyl 3-[(2-hydroxyethyl)(methyl)amino]propanoate

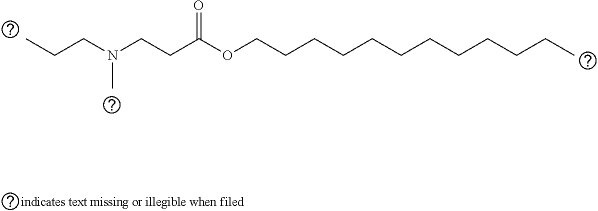 [SMILES: CCCCCCCCCCCCOC(=O)CCN(C)CCC]

A glass vial was charged with 2-(methylamino)ethanol (300 mg, 4.0 mmol) and dodecyl acrylate (960 mg, 4.0 mmol). A mixture of isopropanol and hexafluoroisopropanol (v:v=3:1) was added and the reaction mixture was mixed well. The reaction was heated to 50° C. for 3 hours with vigorous stirring. The solvent was then removed by vacuum. The title compound was collected as a pale-yellow, viscous oil and used without further purification.

### Example 5

### Synthesis of Compounds According to Formula IV

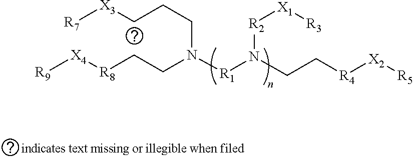

### Representative Procedure 4

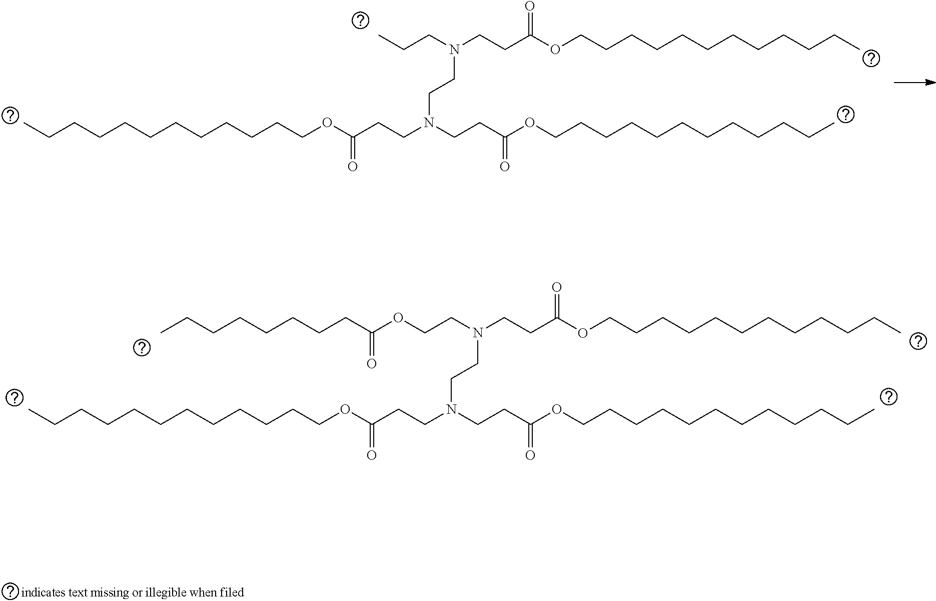

### Didodecyl 3,3′-({3-[nonanoylethyl)(3-dodecoxy-3-oxopropyl)amino]ethyl}azandiyl)dipropanoate (Method E)

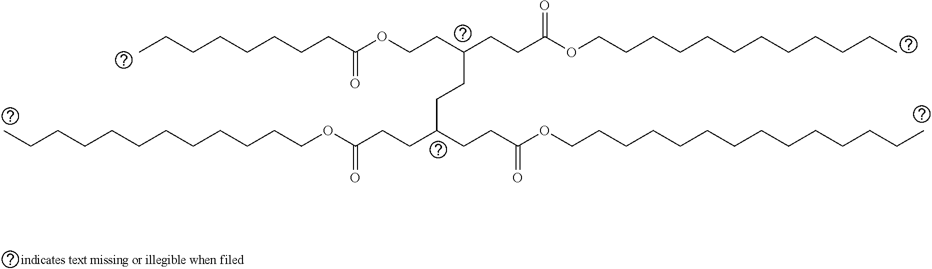 [SMILES: CCCCCCCCCCCCCCOC(=O)CCC(CCC(=O)OCCCCCCCCCCCC)CCC(CCOC(=O)CCCCCCCC)CCC(=O)OCCCCCCCCCCCC]

A glass vial was charged with didodecyl 3,3′-({2-hydroxyethyl)(3-dodecoxy-3-oxopropyl)amino]ethyl}azanediyl)dipropanoate (410 mg, 0.5 mmol) and triethylamine (55 mg, 0.55 mmol). Anhydrous dichloromethane (1 mL) was added and the mixture was stirred. Nonanoyl chloride (93 mg, 1.05 mmol) was diluted with anhydrous dichloromethane (1 mL) and added to the mixture dropwise. The reaction mixture was stirred for 2 hours at room temperature. The reaction was concentrated under vacuum and the residue was mixed with hexanes (5 mL) to precipitate white, needle-like crystals. The mixture was filtered and the hexane was removed under vacuum to yield the title compound as a colorless, viscous oil, which was used directly without further purification.

### Synthesis of Intermediates

### didodecyl 3,3′-({2-hydroxyethyl)(3-dodecoxy-3-oxopropyl)amino]ethyl}azanediyl)dipropanoate

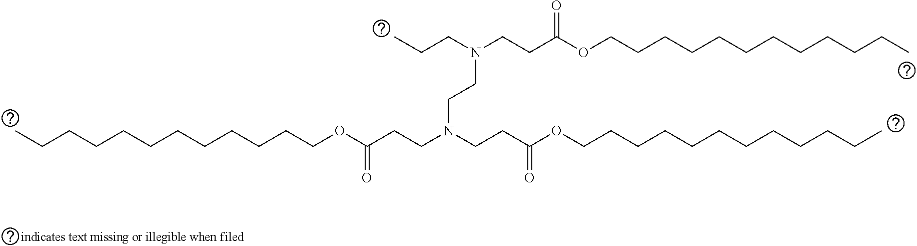 [SMILES: CCCCCCCCCCCCOC(=O)CCN(CCC)CCN(CCC(=O)OCCCCCCCCCCCC)CCC(=O)OCCCCCCCCCCCC]

A glass vial was charged with 2-(2-aminoethylamino)ethanol (104 mg, 1.0 mmol) and dodecyl acrylate (790 mg, 3.3 mmol). A mixture of isopropanol and hexafluoroisopropanol (v:v=3:1) (1 mL) was added and the reaction mixture was mixed well. The reaction was heated to 50° C. for 40 hours with vigorous stirring. The solvent was removed under vacuum to yield the title compound as a colorless, viscous oil, which was used directly without further purification.

### Example 6

### Synthesis of Compound 125

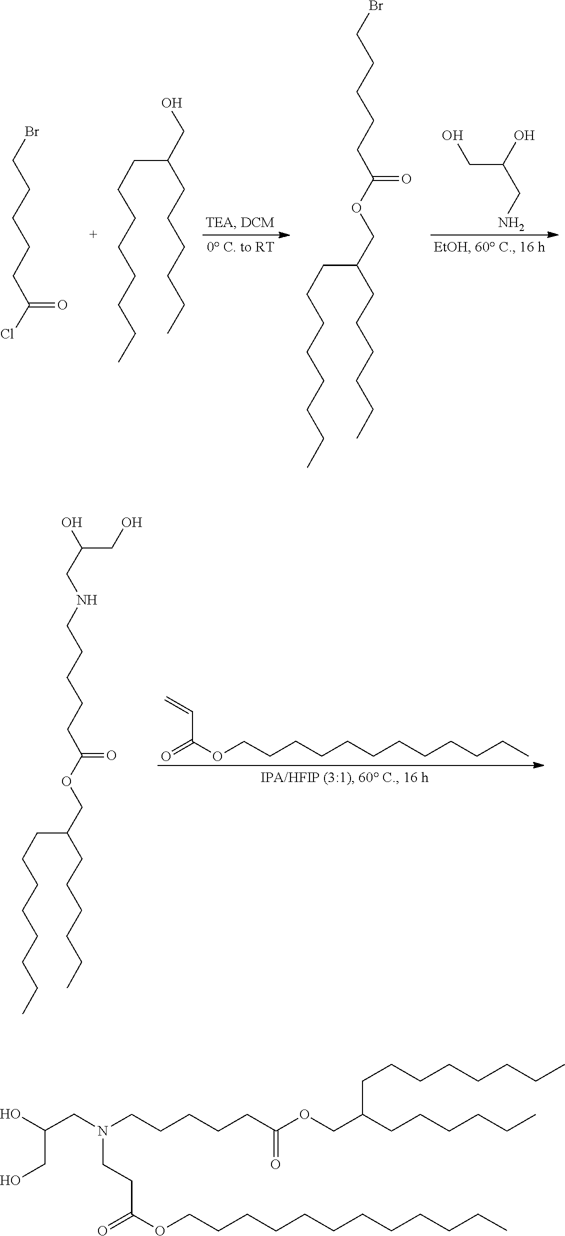

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 3-aminopropane-1,2-diol (3.64 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2,3-dihydroxypropyl)amino)hexanoate as colorless oil (1.2 g, 70%).

A solution of 2-hexyldecyl 6-((2,3-dihydroxypropyl)amino)hexanoate (0.430 mg, 1 mmol) and dodecyl acrylate (240 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 5% MeOH in DCM) to obtain the product (312 mg, 46%) as light brown oil. ¹H NMR (500 MHz, CDCl₃) δ=4.07 (td, J=7.3, 2.8 Hz, 2H), 3.96 (t, J=4.7, Hz, 2H), 3.77-3.70 (m, 2H), 3.50-3.43 (m, 1H), 2.92 (dt, J=13.7, 7.7 Hz, 1H), 2.71 (dd, J=13.4, 6.9 Hz, 1H), 2.58 (dd, J=13.1, 9.5 Hz, 1H), 5.54-2.37 (m, 5H), 2.30 (td, J=8.0, 2.6 Hz, 2H), 1.62 (dq, J=11.3, 5.9 Hz, 5H), 1.46 (s, 2H), 1.38-1.09 (m, 46H), 0.88 (td, J7.4, 2.6 Hz, 9H).

### Example 7

### Synthesis of Compound 226

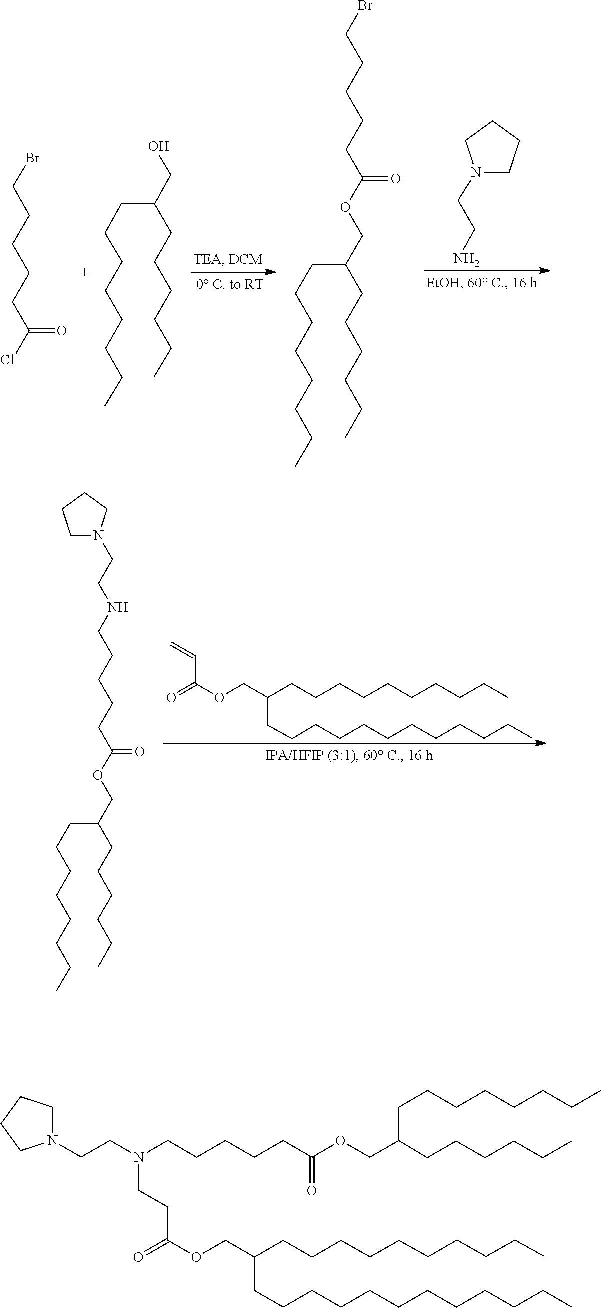

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-pyrrolidinoethylamine (4.56 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-pyrrolidinoethyl)amino)hexanoate as colorless oil (1.22 g, 67%).

A solution of 2-hexyldecyl 6-((2-pyrrolidinoethyl)amino)hexanoate (452 mg, 1 mmol) and 2-decyltetradecyl acrylate (409 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 7.5% MeOH in DCM) to obtain the product (422 mg, 49%). ¹H NMR (500 MHz, CDCl₃) δ=3.95 (dt, J=5.7, 2.6 Hz, 4H), 2.77 J=21.9, 14.9 Hz, 8H), 2.43 (t, J=7.4 Hz, 4H), 2.29 (t, J=7.5 Hz, 2H), 1.89 (s, 4H), 1.61 (q, J=7.6 Hz, 4H), 1.45 (q, J=7.8 Hz, 2H), 1.26 (d, J=6.1 Hz, 68H), 0.92-0.77 (m, 12H).

### Example 8

### Synthesis of Compound 227

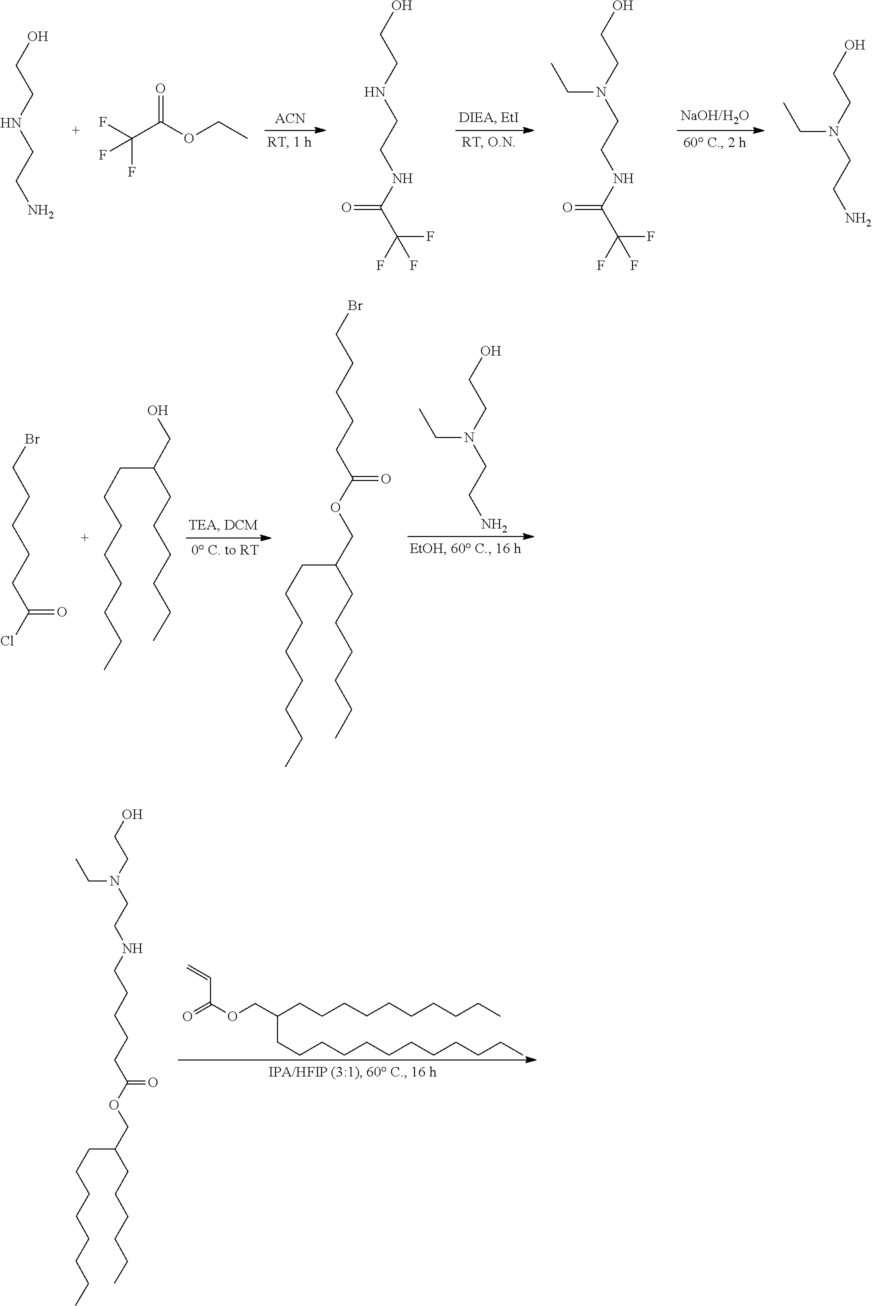

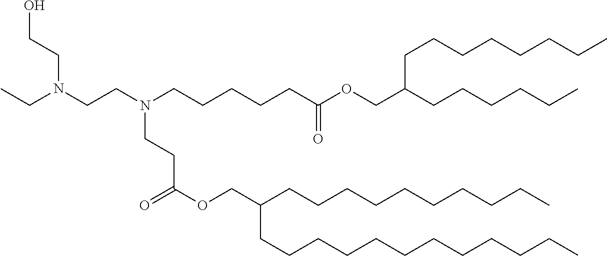 [SMILES: CCCCCCCCCCCCC(CCCCCCCCCC)COC(=O)CCN(CCCCCC(=O)OCC(CCCCCC)CCCCCCCC)CCN(CC)CCO]

Trifluoroacetate (7.1 g, 60 mmol) was added slowly added into a solution of 2-(2-aminoethylamino)ethanol (6.2 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO. The organic solvent was removed under vacuum to afford 2-[(2-aminoethyl)(ethyl)amino]ethan-1-ol as a colorless oil (2.4 g, 30% overall yield). 2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-[(2-aminoethyl)(ethyl)amino]ethan-1-ol (4.22 g, 32 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-hydroxyethyl)(ethyl)aminoethyl)amino)hexanoate as colorless oil (1.18 g, 63%).

A solution of 2-hexyldecyl 6-((2-hydroxyethyl)(ethyl)aminoethyl)amino)hexanoate (470 mg, 1 mmol) and 2-decyltetradecyl acrylate (408 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of NH₄OH, 7.5% MeOH in DCM) to obtain the product (415 mg, 47%). ¹H NMR (500 MHz, CDCl₃) δ=3.96 (d, J=5.7 Hz, 4H), 3.59 (s, 2H), 2.81 (t, J=7.4 Hz, 2H), 2.68 (d, J=17.2 Hz, 5H), 2.58 (s, 2H), 2.46 (dt, J=15.9, 7.6 Hz, 4H), 2.30 (q, J=7.2 Hz, 2H), 1.63 (dt, J=15.7, 7.7 Hz, 4H), 1.48 (pent, J=7.7 Hz, 2H), 1.26 (s, 68H), 1.09 (t, J=7.1 Hz, 3H), 0.88 (t, J=6.7 Hz, 12H).

### Example 9

### Synthesis of Compound 228

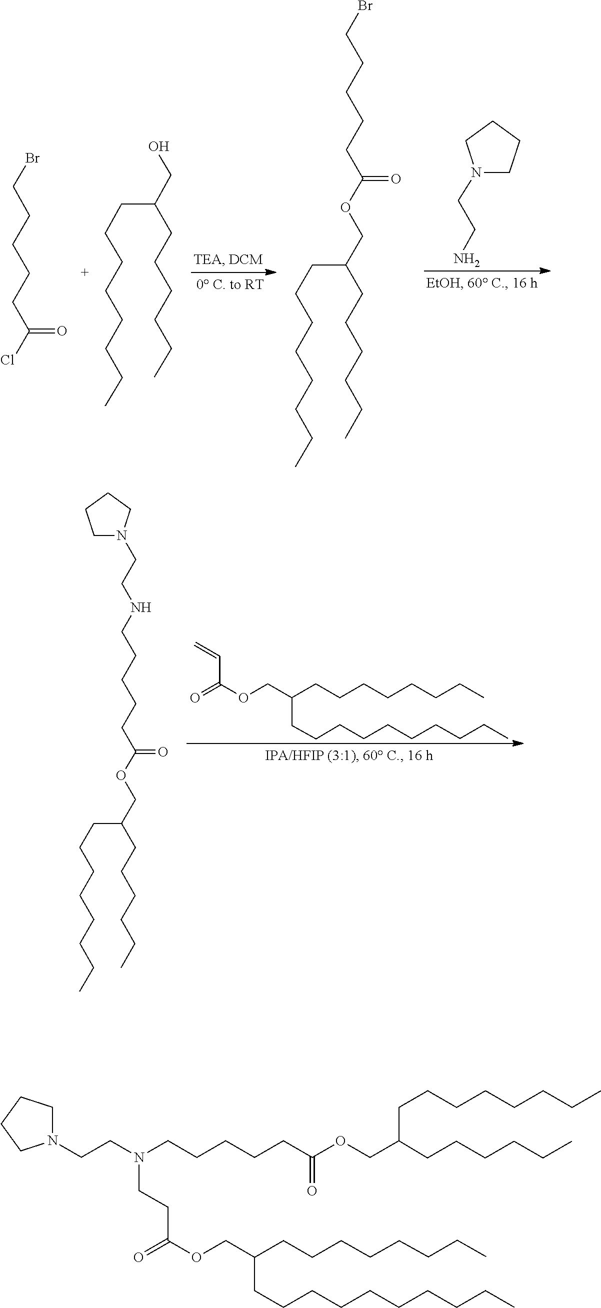

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-pyrrolidinoethylamine (4.56 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-pyrrolidinoethyl)amino)hexanoate as colorless oil (1.22 g, 67%).

A solution of 2-hexyldecyl 6-((2-pyrrolidinoethyl)amino)hexanoate (453 mg, 1 mmol) and 2-octyldodecyl acrylate (352 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 7.5% MeOH in DCM) to obtain the product (398 mg, 49%). ¹H NMR (500 MHz, CDCl₃) δ=3.95 (d, J=5.7 Hz, 4H), 2.80 (t, J=7.3 Hz, 2H), 2.56 (d, J=26.0 Hz, 6H), 2.43 (t, J=7.5 Hz, 4H), 2.29 (t, J=7.5 Hz, 2H), 1.78 (s, 4H), 1.62 (pent, J=7.6 Hz, 4H), 1.45 (pent, J=7.5 Hz, 2H), 1.26 (d, J=5.5 Hz, 60H), 0.87 (t, J=6.7 Hz, 12H).

### Example 10

### Synthesis of Compound 229

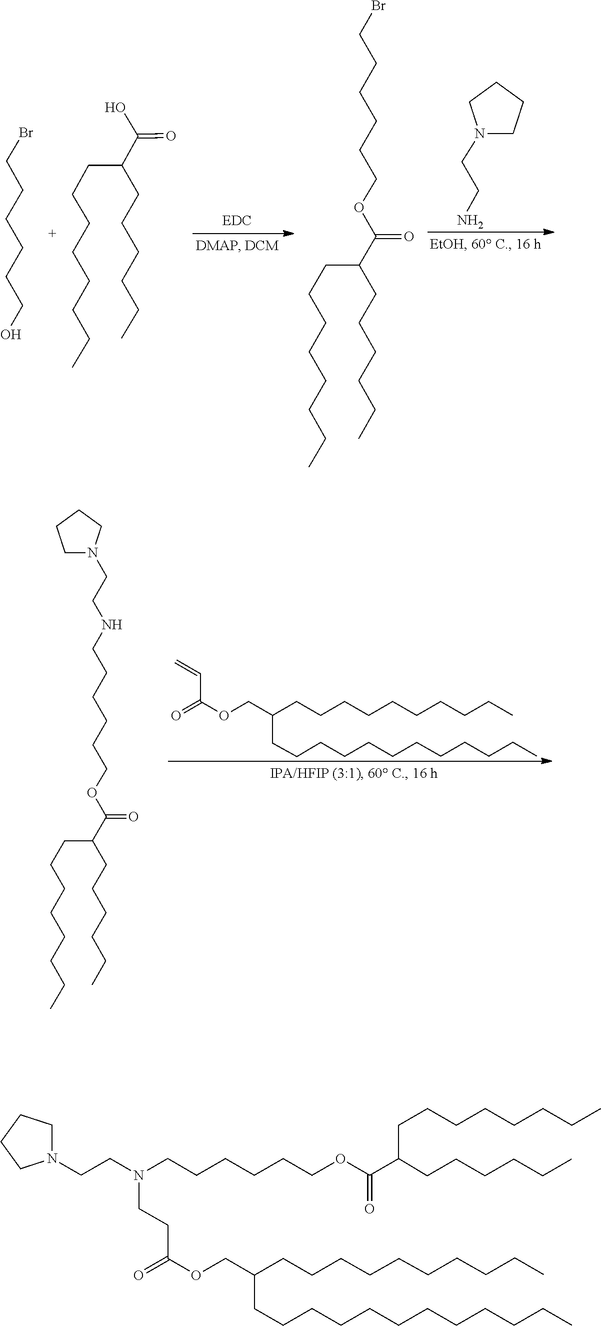

2-Hexyl-1-decanoic acid (3.84 g, 15 mmol) and EDC-HCl (3.74 g, 19.5 mmol) and DMAP (2.38 g, 19.5 mmol) was stirred in anhydrous DCM for 20 minutes at room temperature. 6-Bromo-1-hexanol (2.72 g, 15 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL). The hexane layer was separated, dried over anhydrous Na₂SO₄, and evaporated under vacuum. The residue was purified by silica-gel chromatography (3% ethyl acetate in hexane) to afford 6-bromohexyl 2-hexyldecanoate as colorless oil (2.58 g, 41%).

A solution of 6-bromohexyl 2-hexyldecanoate (2.09 g, 5 mmol) and 2-pyrrolidinoethylamine (5.70 g, 50 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 6-((2-pyrrolidinoethyl)amino)hexyl 2-hexyldecanoate as colorless oil (1.61 g, 71%).

A solution of 6-((2-pyrrolidinoethyl)amino)hexyl 2-hexyldecanoate (452 mg, 1 mmol) and 2-decyltetradecyl acrylate (408 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 7.5% MeOH in DCM) to obtain the product (442 mg, 51%). ¹H NMR (500 MHz, CDCl₃) δ=4.04 (t, J=6.7 Hz, 2H), 3.95 (d, J=5.8 Hz, 2H), 2.80 (t, J=7.3 Hz, 2H), 2.63 (s, 6H), 2.43 (td, J=7.4, 3.2 Hz, 4H), 2.30 (tt, J=9.4, 5.3 Hz, 1H), 1.81 (s, 3H), 1.59 (tt, J=14.4, 7.4 Hz, 5H), 1.43 (h, J=7.6 Hz, 4H), 1.35 (pent, J=7.4 Hz, 66H), 0.87 (td, J=6.9, 2.9 Hz, 12H).

### Example 11

### Synthesis of Compound 230

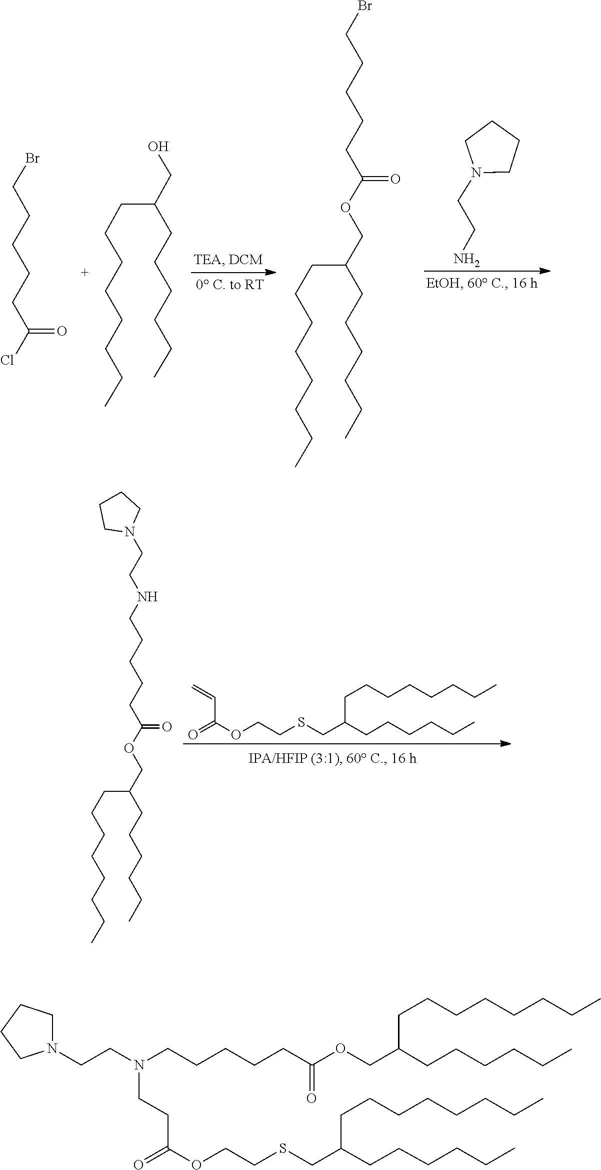

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-pyrrolidinoethylamine (4.56 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-pyrrolidinoethyl)amino)hexanoate as colorless oil (1.22 g, 67%).

A solution of 2-hexyldecyl 6-((2-pyrrolidinoethyl)amino)hexanoate (453 mg, 1 mmol) and 2-((2-hexyldecyl)thio)ethyl acrylate (357 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 7.5% MeOH in DCM) to obtain the product (439 mg, 54%). ¹H NMR (500 MHz, CDCl₃) δ=4.19 (td, J=7.2, 1.8 Hz, 1H), 3.95 (dd, J=5.7, 1.8 Hz, 2H), 2.90-2.60 (m, 11H), 2.52 (dd, J=6.4, 1.8 Hz, 2H), 2.48-2.39 (m, 4H), 2.29 (tt, J=7.5, 4.1 Hz, 2H), 1.87 (s, 4H), 1.62 (pent, J=7.7 Hz, 3H), 10.47 (dq, J=30.7, 6.9 Hz, 3H), 1.26 (d, J=10.2 Hz, 51H), 0.87 (t, J=6.7 Hz, 12H).

### Example 12

### Synthesis of Compound 231

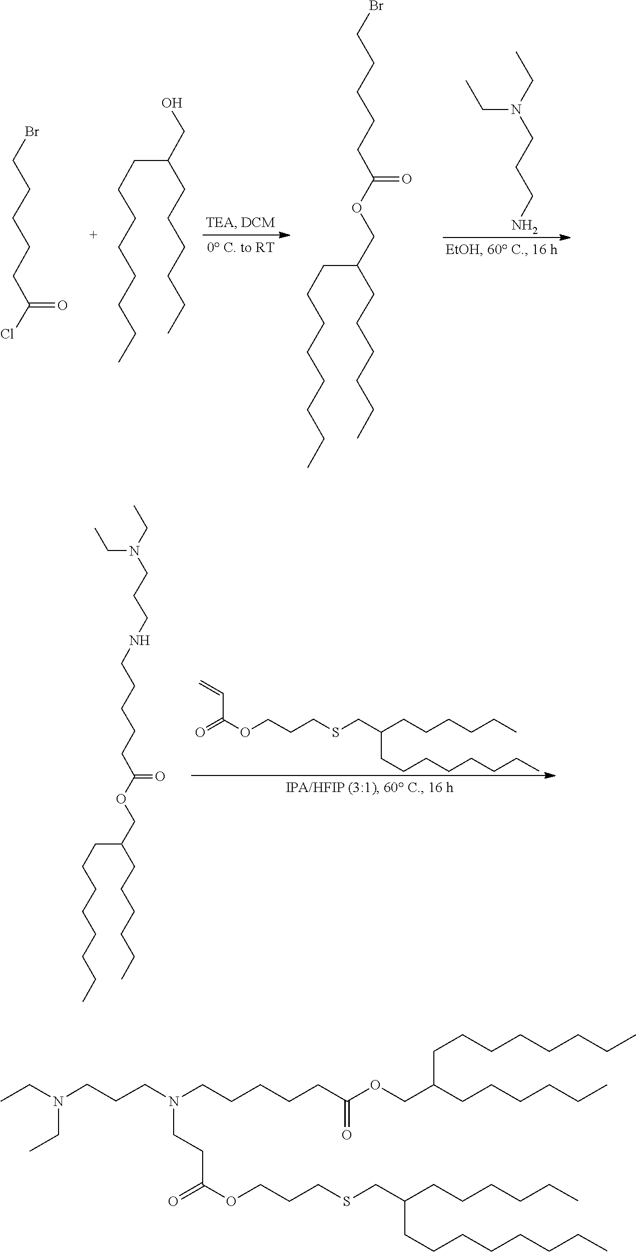

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 3-(N,N-diethylamino)ethylamine (5.20 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-pyrrolidinoethyl)amino)hexanoate as colorless oil (1.24 g, 66%).

A solution of 2-hexyldecyl 6-((3-(N,N-diethylamino)ethyl)amino)hexanoate (468 mg, 1 mmol) and 3-((2-hexyldecyl)thio)propyl acrylate (370 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 7.5% MeOH in DCM) to obtain the product (395 mg, 47%).

### Example 13

### Synthesis of Compound 232

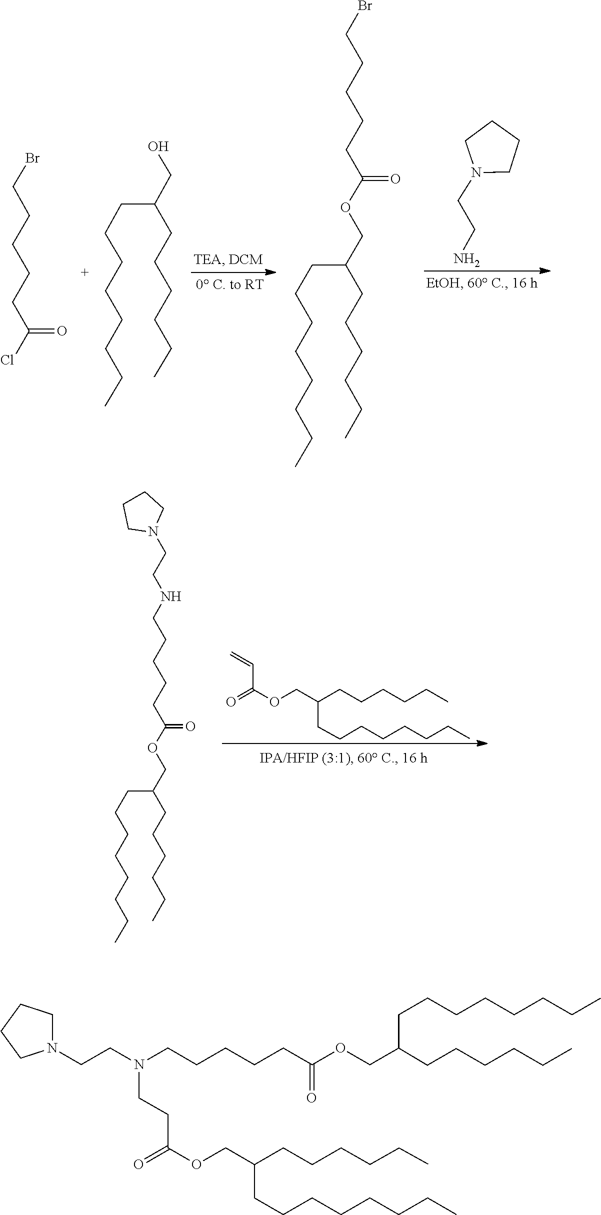

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-pyrrolidinoethylamine (4.56 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-pyrrolidinoethyl)amino)hexanoate as colorless oil (1.22 g, 67%).

A solution of 2-hexyldecyl 6-((2-pyrrolidinoethyl)amino)hexanoate (452 mg, 1 mmol) and 2-hexyldecyl acrylate (296 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 7.5% MeOH in DCM) to obtain the product (348 mg, 46%). ¹H NMR (500 MHz, CDCl₃) δ=3.95 (d, J=5.8 Hz, 4H), 2.79 (t, J=7.2 Hz, 2H), 2.67 (s, 6H), 2.43 (t, J=7.4 Hz, 4H), 2.29 (t, J=7.5 Hz, 4H), 1.84 (s, 4H), 1.62 (pent, J=7.6 Hz, 4H), 1.45 (pent, J=7.7 Hz, 2H), 1.26 (d, J=4.7 Hz, 52H), 0.87 (t, J=6.7 Hz, 12H).

### Example 14

### Synthesis of Compound 233

Trifluoroacetate (7.1 g, 60 mmol) was added slowly added into a solution of 2-(2-aminoethylamino)ethanol (6.2 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO. The organic solvent was removed under vacuum to afford 2-[(2-aminoethyl)(ethyl)amino]ethan-1-ol as a colorless oil (2.4 g, 30% overall yield).

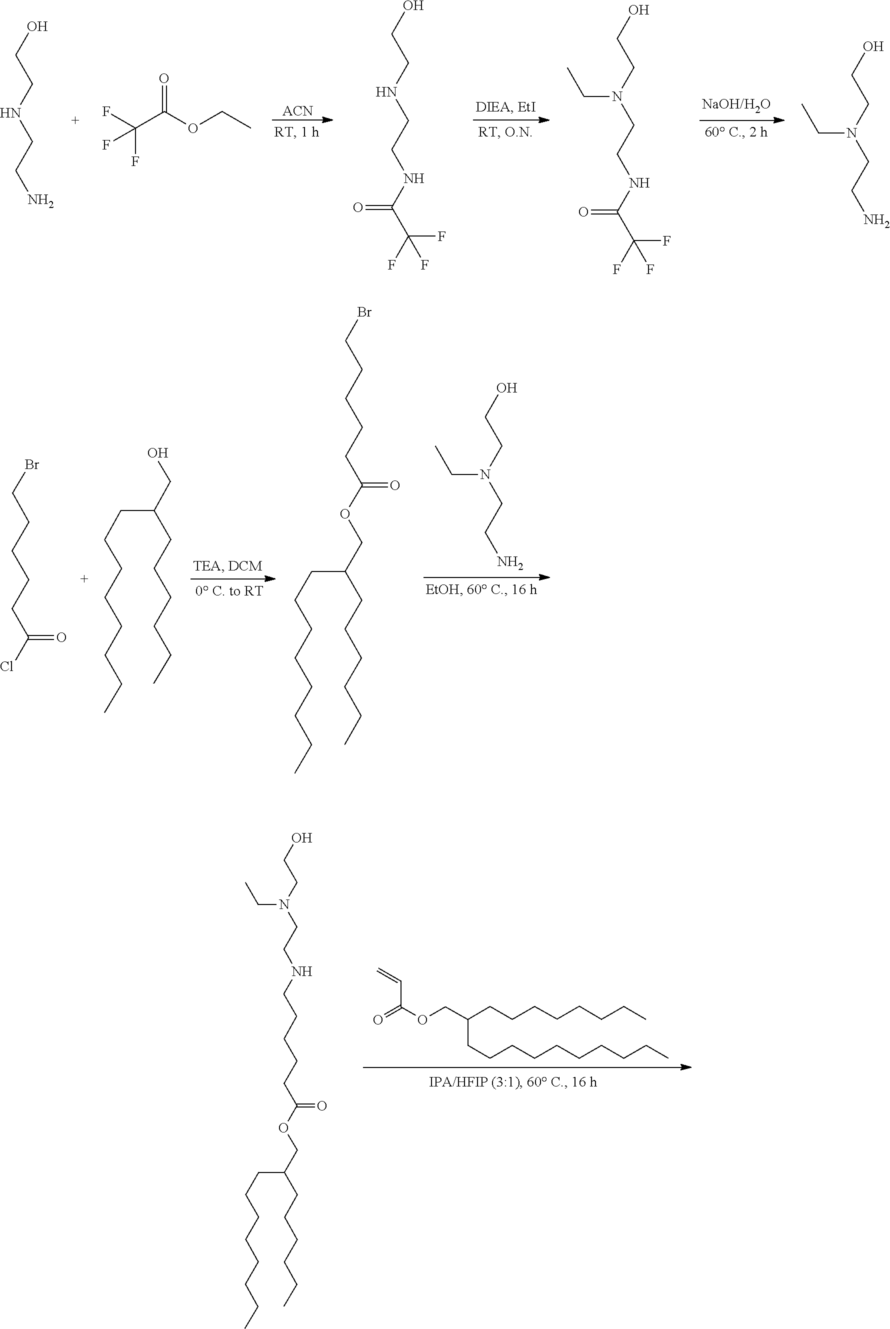

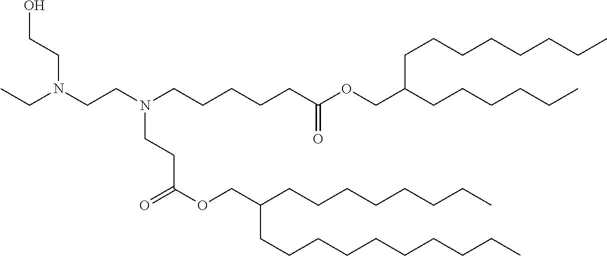 [SMILES: CCCCCCCCCCC(CCCCCCCC)COC(=O)CCN(CCCCCC(=O)OCC(CCCCCC)CCCCCCCC)CCN(CC)CCO]

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-[(2-aminoethyl)(ethyl)amino]ethan-1-ol (4.22 g, 32 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-hydroxyethyl)(ethyl)aminoethyl)amino)hexanoate as colorless oil (1.18 g, 63%).

A solution of 2-hexyldecyl 6-((2-hydroxyethyl)(ethyl)aminoethyl)amino)hexanoate (470 mg, 1 mmol) and 2-octyldodecyl acrylate (352 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 7.5% MeOH in DCM) to obtain the product (356 mg, 43%). ¹H NMR (500 MHz, CDCl₃) δ=3.96 (d, J=5.7 Hz, 4H), 3.63 (s, 2H), 2.82 (t, J=7.3 Hz, 2H), 2.79-2.66 (m, 6H), 2.62 (s, 2H), 2.47 (dt, J=14.0, 7.4 Hz, 3H), 2.30 (d, J=7.9 Hz, 2H), 1.63 (dt, J=16.1, 7.7 Hz, 5H), 1.48 (pent, J=8.1 Hz, 2H), 1.26 (d, J=11.0 Hz, 59H), 1.14-1.06 (m, 3H), 0.87 (t, J=6.8 Hz, 12H).

### Example 15

### Synthesis of Compound 234

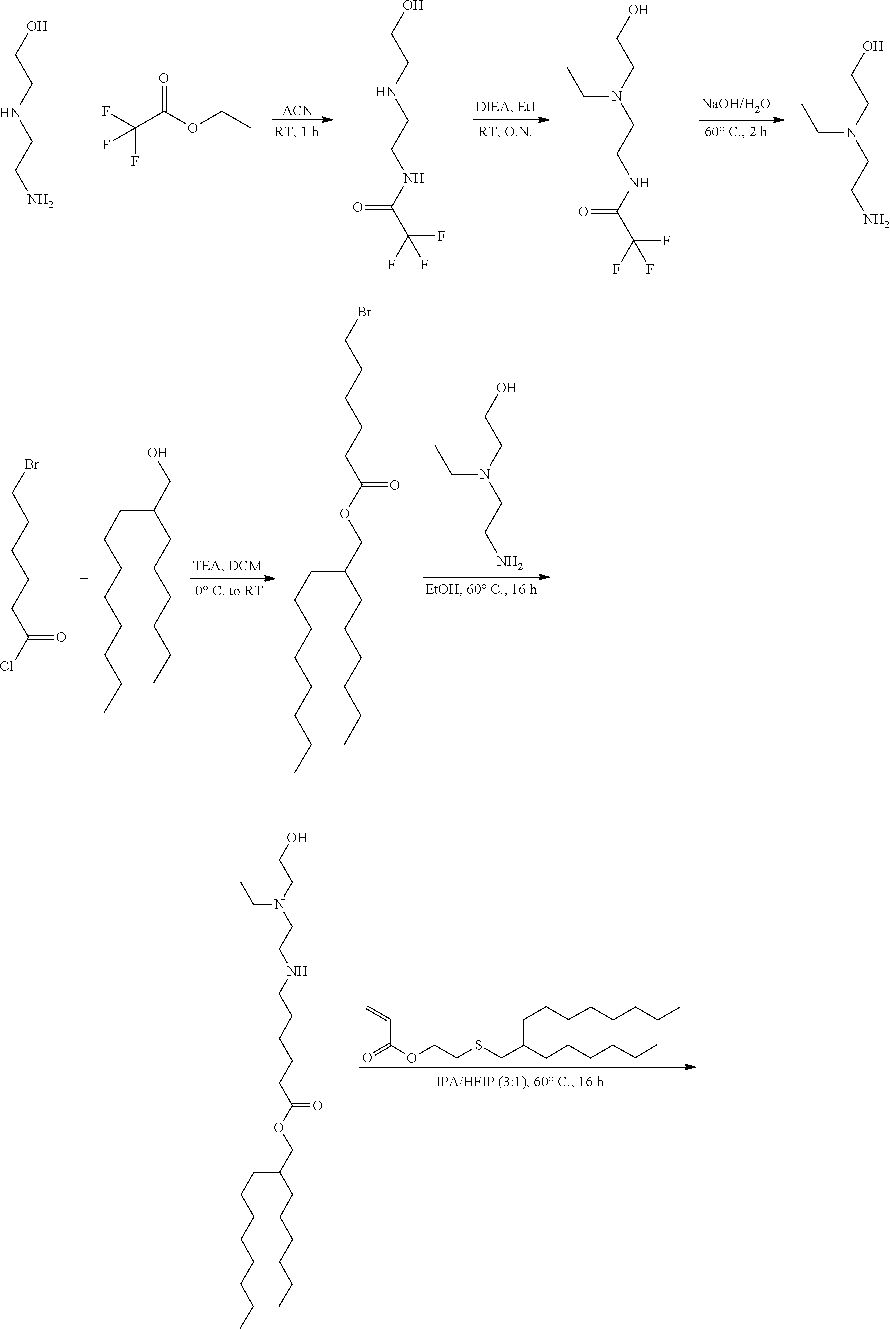

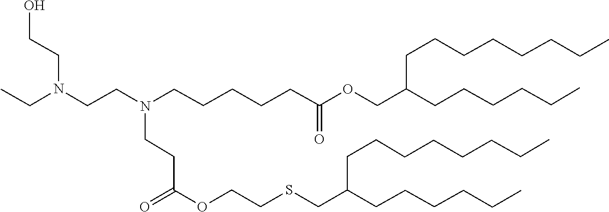 [SMILES: CCCCCCCCC(CCCCCC)COC(=O)CCCCCN(CCC(=O)OCCSCC(CCCCCC)CCCCCCCC)CCN(CC)CCO]

Trifluoroacetate (7.1 g, 60 mmol) was added slowly added into a solution of 2-(2-aminoethylamino)ethanol (6.2 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO. The organic solvent was removed under vacuum to afford 2-[(2-aminoethyl)(ethyl)amino]ethan-1-ol as a colorless oil (2.4 g, 30% overall yield). 2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-[(2-aminoethyl)(ethyl)amino]ethan-1-ol (4.22 g, 32 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-hydroxyethyl)(ethyl)aminoethyl)amino)hexanoate as colorless oil (1.18 g, 63%).

A solution of 2-hexyldecyl 6-((2-hydroxyethyl)(ethyl)aminoethyl)amino)hexanoate (470 mg, 1 mmol) and 2-((2-hexyldecyl)thio)ethyl acrylate (356 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 7.5% MeOH in DCM) to obtain the product (332 mg, 40%). ¹H NMR (500 MHz, CDCl₃) δ=4.20 (t, J=7.1 Hz, 2H), 3.96 (d, J=5.7 Hz, 2H), 3.52 (t, J=5.1 Hz, 2H), 2.81 (t, J=7.5 Hz, 2H), 2.70 (t, J=7.1 Hz, 2H), 2.59 (td, J=16.5, 6.5 Hz, 6H), 2.54-2.45 (m, 6H), 2.42 (t, J=7.8 Hz, 2H), 2.29 (t, J=7.5 Hz, 2H), 1.63 (pent, J=7.5 Hz, 3H), 1.49 (dq, J=23.6, 6.9 Hz, 3H), 1.27 (d, J=9.8 Hz, 51H), 1.04 (t, J=7.0 Hz, 3H), 0.88 (t, J=6.7 Hz, 12H).

### Example 16

### Synthesis of Compound 235

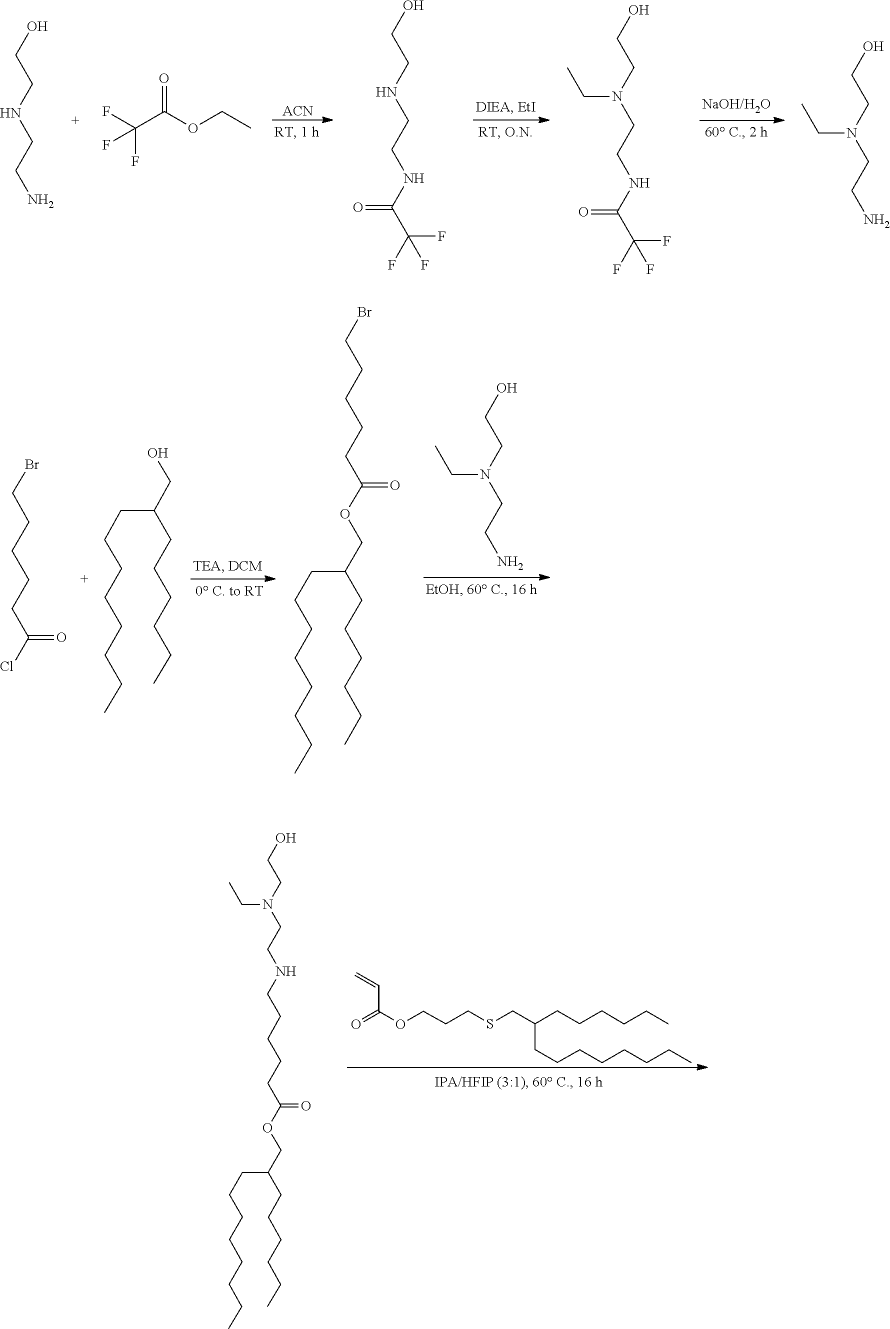

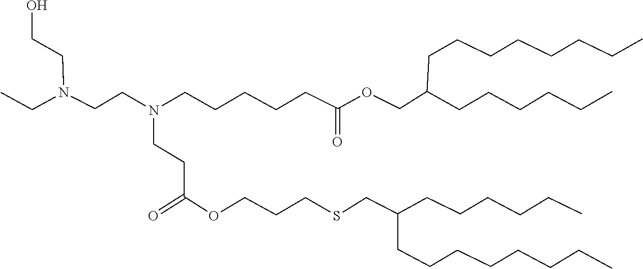 [SMILES: CCCCCCCCC(CCCCCC)COC(=O)CCCCCN(CCC(=O)OCCCSCC(CCCCCC)CCCCCCCC)CCN(CC)CCO]

Trifluoroacetate (7.1 g, 60 mmol) was added slowly added into a solution of 2-(2-aminoethylamino)ethanol (6.2 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO. The organic solvent was removed under vacuum to afford 2-[(2-aminoethyl)(ethyl)amino]ethan-1-ol as a colorless oil (2.4 g, 30% overall yield).

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-[(2-aminoethyl)(ethyl)amino]ethan-1-ol (4.22 g, 32 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-hydroxyethyl)(ethyl)aminoethyl)amino)hexanoate as colorless oil (1.18 g, 63%).

A solution of 2-hexyldecyl 6-((2-hydroxyethyl)(ethyl)aminoethyl)amino)hexanoate (470 mg, 1 mmol) and 2-((2-hexyldecyl)thio)propyl acrylate (370 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 7.5% MeOH in DCM) to obtain the product (365 mg, 43%).

### Example 17

### Synthesis of Compound 236

Trifluoroacetate (7.1 g, 60 mmol) was added slowly added into a solution of 2-(2-aminoethylamino)ethanol (6.2 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO. The organic solvent was removed under vacuum to afford 2-[(2-aminoethyl)(ethyl)amino]ethan-1-ol as a colorless oil (2.4 g, 30% overall yield).

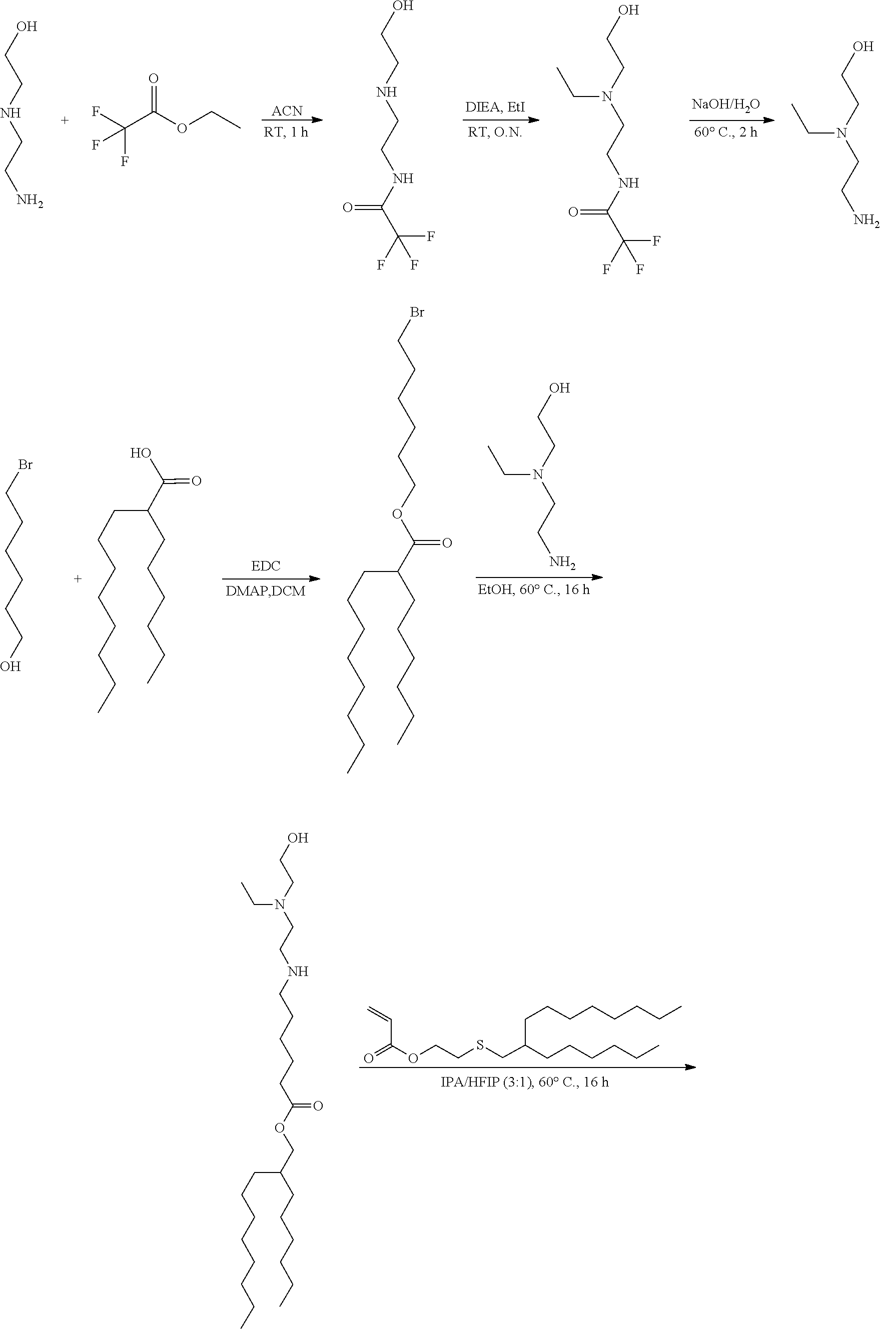

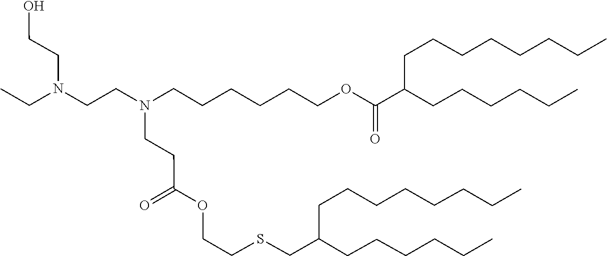 [SMILES: CCCCCCCCC(CCCCCC)CSCCOC(=O)CCN(CCCCCCOC(=O)C(CCCCCC)CCCCCCCC)CCN(CC)CCO]

2-Hexyl-1-decanoic acid (3.84 g, 15 mmol) and EDC-HCl (3.74 g, 19.5 mmol) and DMAP (2.38 g, 19.5 mmol) was stirred in anhydrous DCM for 20 minutes at room temperature. 6-Bromo-1-hexanol (2.72 g, 15 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL). The hexane layer was separated, dried over anhydrous Na₂SO₄, and evaporated under vacuum. The residue was purified by silica-gel chromatography (3% ethyl acetate in hexane) to afford 6-bromohexyl 2-hexyldecanoate as colorless oil (2.58 g, 41%).

A solution of 6-bromohexyl 2-hexyldecanoate (2.10 g, 5 mmol) and 2-[(2-aminoethyl)(ethyl)amino]ethan-1-ol (5.2 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 6-(((2-hydroxyethyl)(ethyl)aminoethyl)amino)hexyl 2-hexyldecanoate as colorless oil (1.54 g, 66%).

A solution of 6-(((2-hydroxyethyl)(ethyl)aminoethyl)amino)hexyl 2-hexyldecanoate (470 mg, 1 mmol) and 2-((2-hexyldecyl)thio)ethyl acrylate (356 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 7.5% MeOH in DCM) to obtain the product (376 mg, 45%).

### Example 18

### Synthesis of Compound 237

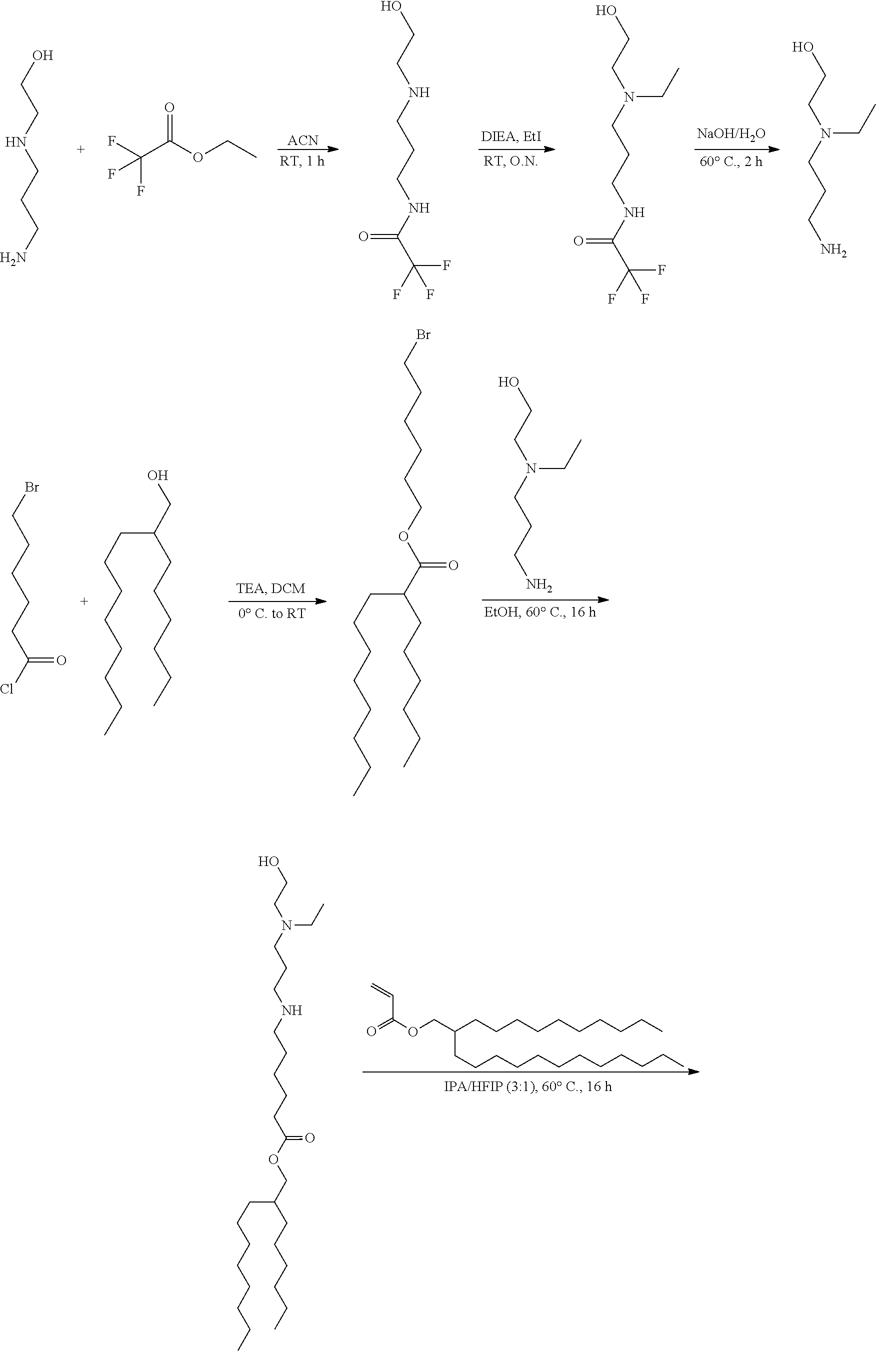

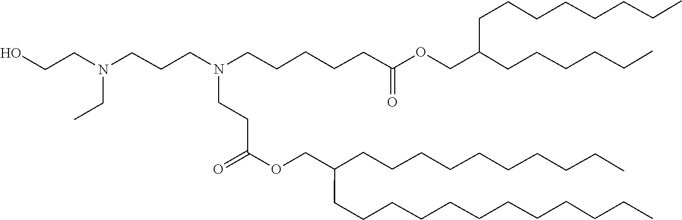 [SMILES: CCCCCCCCCCCCC(CCCCCCCCCC)COC(=O)CCN(CCCCCC(=O)OCC(CCCCCC)CCCCCCCC)CCCN(CC)CCO]

Trifluoroacetate (7.1 g, 60 mmol) was added slowly added into a solution of 2-((3-aminopropyl)amino)ethanol (7.08 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO₄. The organic solvent was removed under vacuum to afford 2-((3-aminopropyl)(ethyl)amino)ethanol as a colorless oil (2.8 g, 31% overall yield). 2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (0.84 g, 2 mmol) and 2-((3-aminopropyl)(ethyl)amino)ethanol (2.33 g, 16 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-hydroxyethyl)(ethyl)aminopropyl)amino)hexanoate as colorless oil (0.66 g, 68%).

A solution of 2-hexyldecyl 6-((2-hydroxyethyl)(ethyl)aminopropyl)amino)hexanoate (97 mg, 0.2 mmol) and 2-decyltetradecyl acrylate (82 mg, 0.2 mmol) in a mixture of isopropanol/hexafluoroisopropanol (v:v, 3:1, 2 mL) was stirred at 60° C. for 16 hours. The solvents were removed to obtain the product.

### Example 19

### Synthesis of Compound 238

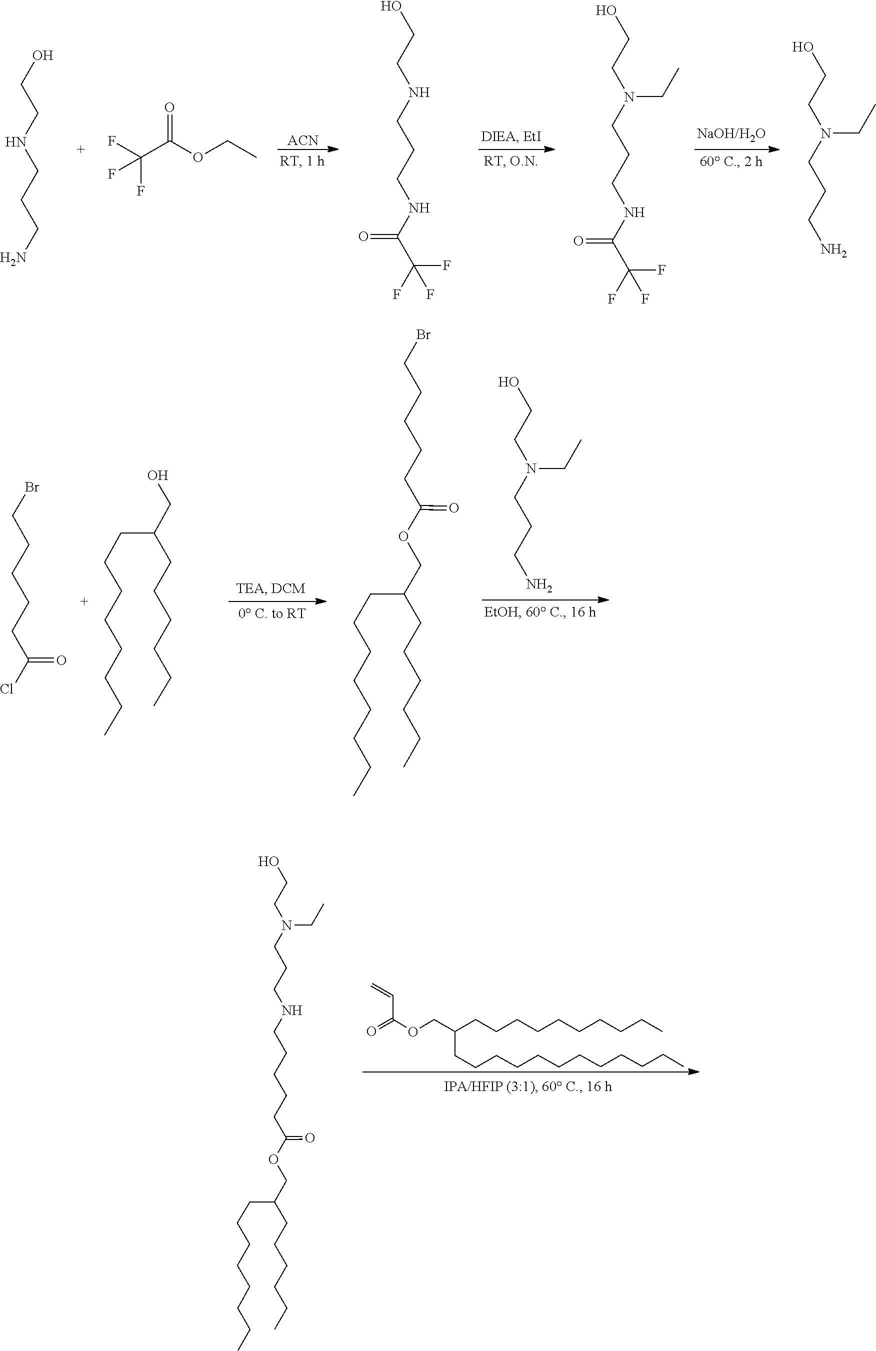

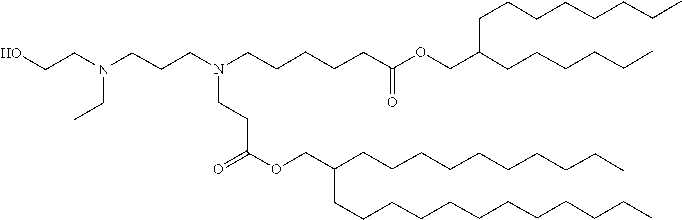 [SMILES: CCCCCCCCCCCCC(CCCCCCCCCC)COC(=O)CCN(CCCCCC(=O)OCC(CCCCCC)CCCCCCCC)CCCN(CC)CCO]

Trifluoroacetate (7.1 g, 60 mmol) was added slowly added into a solution of 2-((3-aminopropyl)amino)ethanol (7.08 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO₄. The organic solvent was removed under vacuum to afford 2-((3-aminopropyl)(ethyl)amino)ethanol as a colorless oil (2.8 g, 31% overall yield). 2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (0.84 g, 2 mmol) and 2-((3-aminopropyl)(ethyl)amino)ethanol (2.33 g, 16 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-hydroxyethyl)(ethyl)aminopropyl)amino)hexanoate as colorless oil (0.66 g, 68%).

A solution of 2-hexyldecyl 6-((2-hydroxyethyl)(ethyl)aminopropyl)amino)hexanoate (96 mg, 0.2 mmol) and 2-octyldodecyl acrylate 70 mg, 0.2 mmol) in a mixture of isopropanol/hexafluoroisopropanol (v:v, 3:1, 2 mL) was stirred at 60° C. for 16 hours. The solvents were removed to obtain the product.

### Example 20

### Synthesis of Compound 239

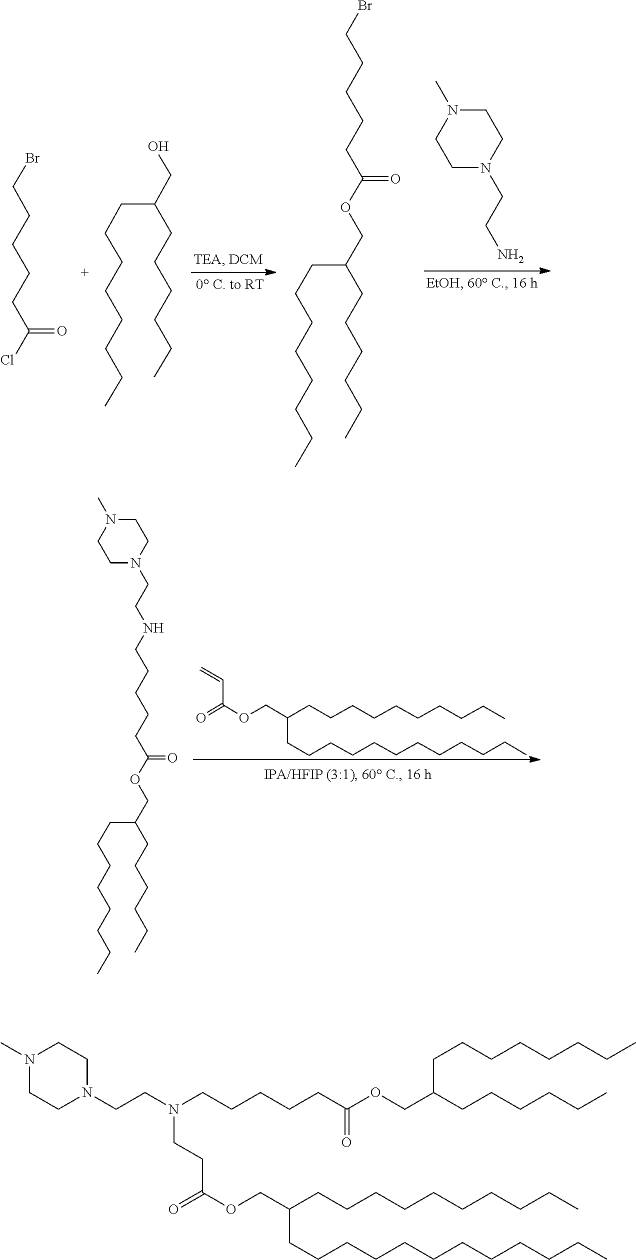

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-(4-methyl-piperazin-1-yl)-ethylamine (5.72 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-(4-methyl-piperazin-1-yl)-ethyl)amino)hexanoate as colorless oil (1.26 g, 65%).

A solution of 2-hexyldecyl 6-((2-(4-methyl-piperazin-1-yl)-ethyl)amino)hexanoate (96 mg, 0.2 mmol) and 2-decyltetradecyl acrylate (82 mg, 0.2 mmol) in a mixture of isopropanol/hexafluoroisopropanol (v:v, 3:1, 2 mL) was stirred at 60° C. for 16 hours. The solvents were removed to obtain the product.

### Example 21

### Synthesis of Compound 240

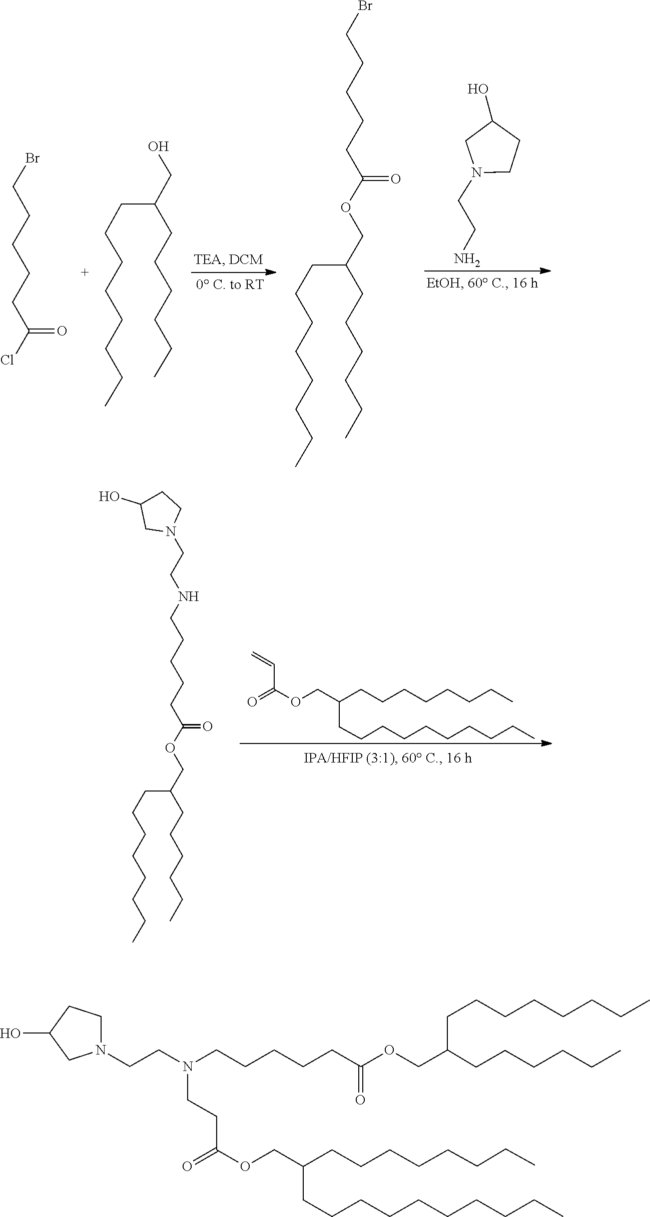

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-(3-hydroxypyrrolidino)ethylamine (5.20 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 6-((2-(3-hydroxypyrrolidino)ethyl)amino)hexanoate as colorless oil (1.18 g, 63%).

A solution of 2-hexyldecyl 6-((2-(3-hydroxypyrrolidino)ethyl)amino)hexanoate (94 mg, 0.2 mmol) and 2-octyldodecyl acrylate (70 mg, 0.2 mmol) in a mixture of isopropanol/hexafluoroisopropanol (v:v, 3:1, 2 mL) was stirred at 60° C. for 16 hours. The solvents were removed to obtain the product.

### Example 22

### Synthesis of Compound 241

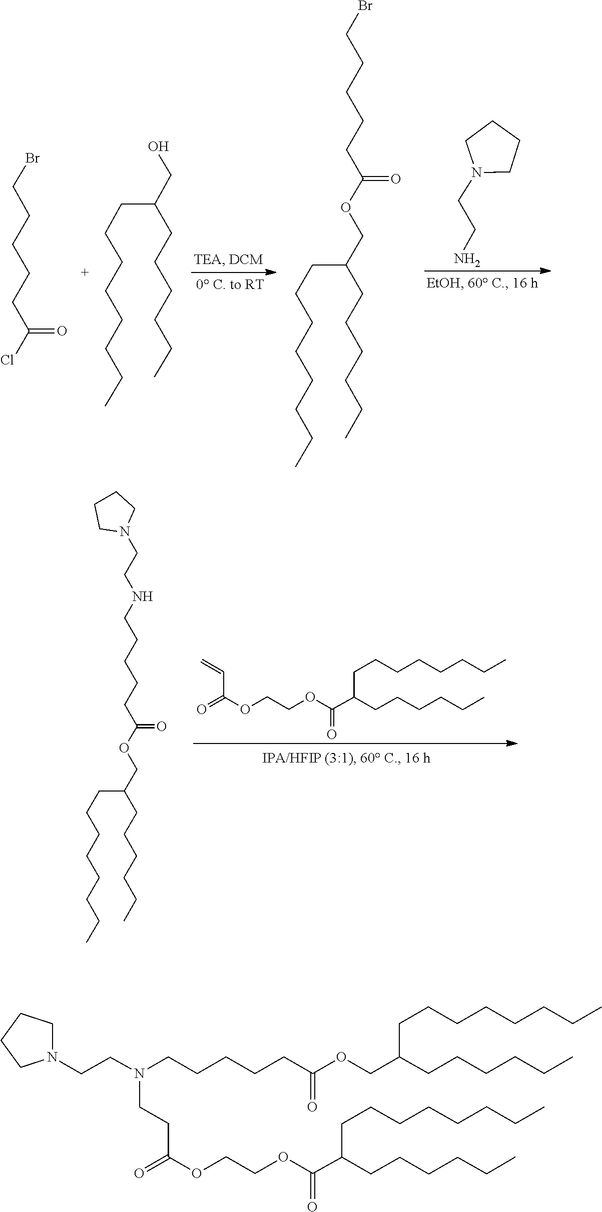

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-pyrrolidinoethylamine (4.56 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-pyrrolidinoethyl)amino)hexanoate as colorless oil (1.22 g, 67%).

A solution of 2-hexyldecyl 6-((2-pyrrolidinoethyl)amino)hexanoate (91 mg, 0.2 mmol) and 2-((2-hexyldecyl)oxy)ethyl acrylate (71 mg, 0.2 mmol) in a mixture of isopropanol/hexafluoroisopropanol (v:v, 3:1, 2 mL) was stirred at 60° C. for 16 hours. The solvents were removed to obtain the product.

### Example 23

### Synthesis of Compound 242

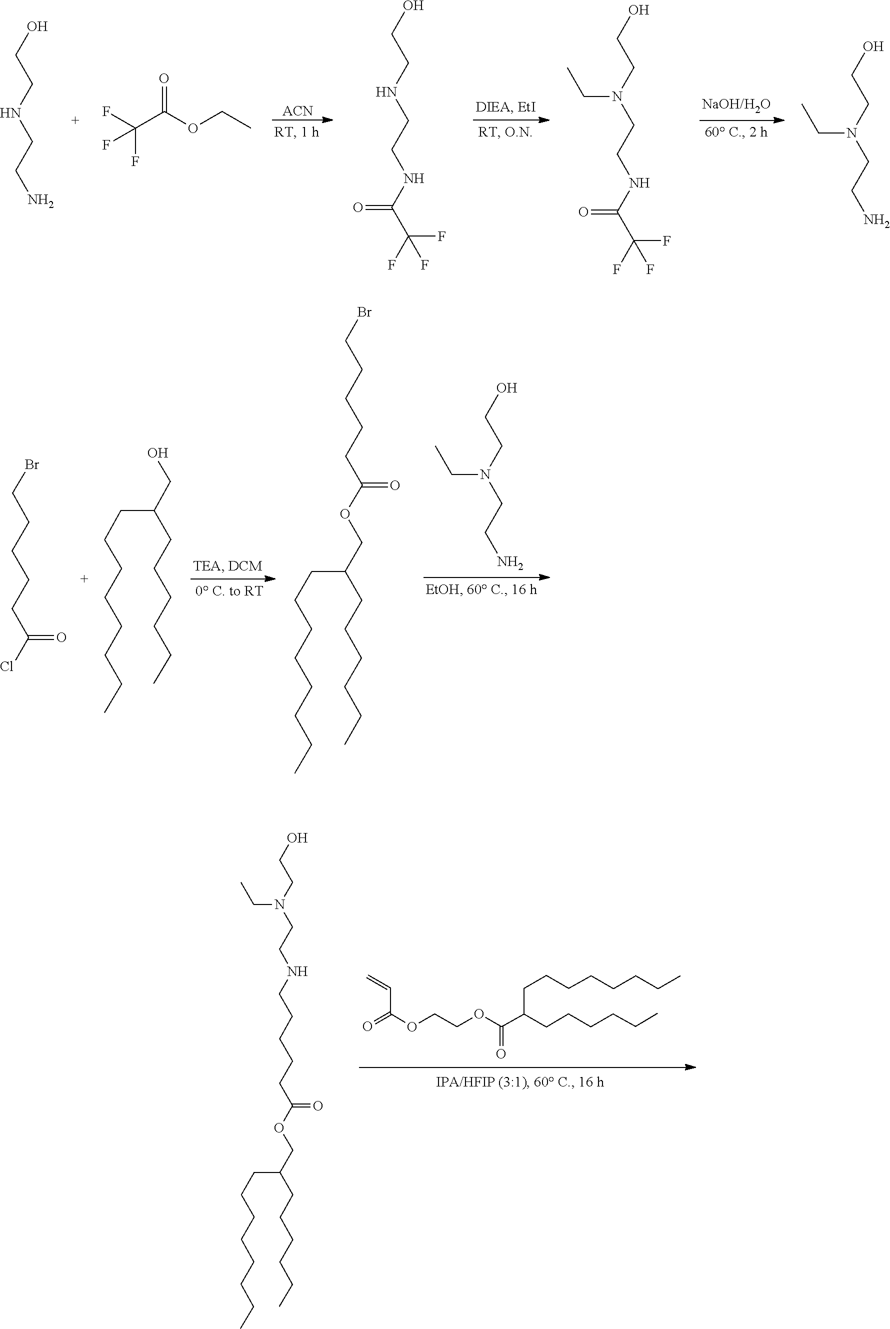

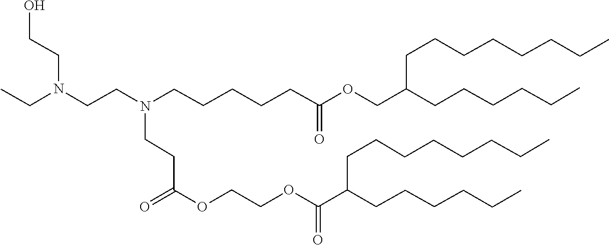 [SMILES: CCCCCCCCC(CCCCCC)COC(=O)CCCCCN(CCC(=O)OCCOC(=O)C(CCCCCC)CCCCCCCC)CCN(CC)CCO]

Trifluoroacetate (7.1 g, 60 mmol) was added slowly added into a solution of 2-(2-aminoethylamino)ethanol (6.2 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO. The organic solvent was removed under vacuum to afford 2-[(2-aminoethyl)(ethyl)amino]ethan-1-ol as a colorless oil (2.4 g, 30% overall yield). 2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-[(2-aminoethyl)(ethyl)amino]ethan-1-ol (4.22 g, 32 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-hydroxyethyl)(ethyl)aminoethyl)amino)hexanoate as colorless oil (1.18 g, 63%).

A solution of 2-hexyldecyl 6-((2-hydroxyethyl)(ethyl)aminoethyl)amino)hexanoate (93 mg, 0.2 mmol) and 2-((2-hexyldecyl)oxy)ethyl acrylate (71 mg, 0.2 mmol) in a mixture of isopropanol/hexafluoroisopropanol (v:v, 3:1, 2 mL) was stirred at 60° C. for 16 hours. The solvents were removed to obtain the product.

### Example 24

### Synthesis of Compound 243

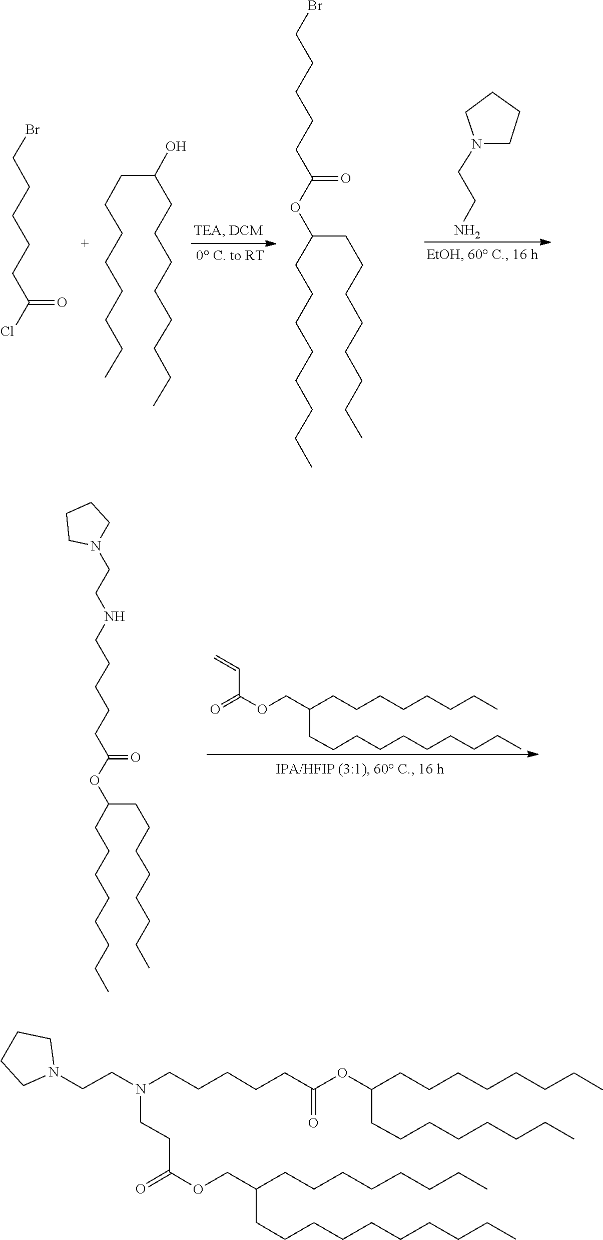

9-heptadecanol (2.56 g, 10 mmol) and TEA (1.21 g, 12 mmol) was mixed in anhydrous DCM (20 mL) and cooled to 0° C. 6-Bromohexanoyl chloride (2.14 g, 10 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 9-heptadecyl 6-bromohexanoate as colorless liquid (3.68 g, 85%).

A solution of afford 9-heptadecyl 6-bromohexanoate (1.72 g, 4 mmol) and 2-pyrrolidinoethylamine (4.56 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 9-heptadecyl 6-((2-pyrrolidinoethyl)amino)hexanoate as colorless oil (1.32 g, 71%).

A solution of 9-heptadecyl 6-((2-pyrrolidinoethyl)amino)hexanoate (93 mg, 0.2 mmol) and 2-octyldodecyl acrylate (70 mg, 0.2 mmol) in a mixture of isopropanol/hexafluoroisopropanol (v:v, 3:1, 2 mL) was stirred at 60° C. for 16 hours. The solvents were removed to afford the crude product. ¹H NMR (500 MHz, CDCl₃) δ 4.89-4.82 (m, 1H), 3.96 (t, J=4.1 Hz, 2H), 2.80 (t, J=7.4 Hz, 2H), 2.59 (d, J=21.4 Hz, 7H), 2.43 (t, J=7.5 Hz, 4H), 2.30-2.24 (m, 2H), 1.79 (s, 4H), 1.61 (q, J=7.7 Hz, 3H), 1.54-1.40 (m, 6H), 1.26 (d, J=9.2 Hz, 59H), 0.87 (td, J=6.9, 3.3 Hz, 12H).

### Example 25

### Synthesis of Compound 244

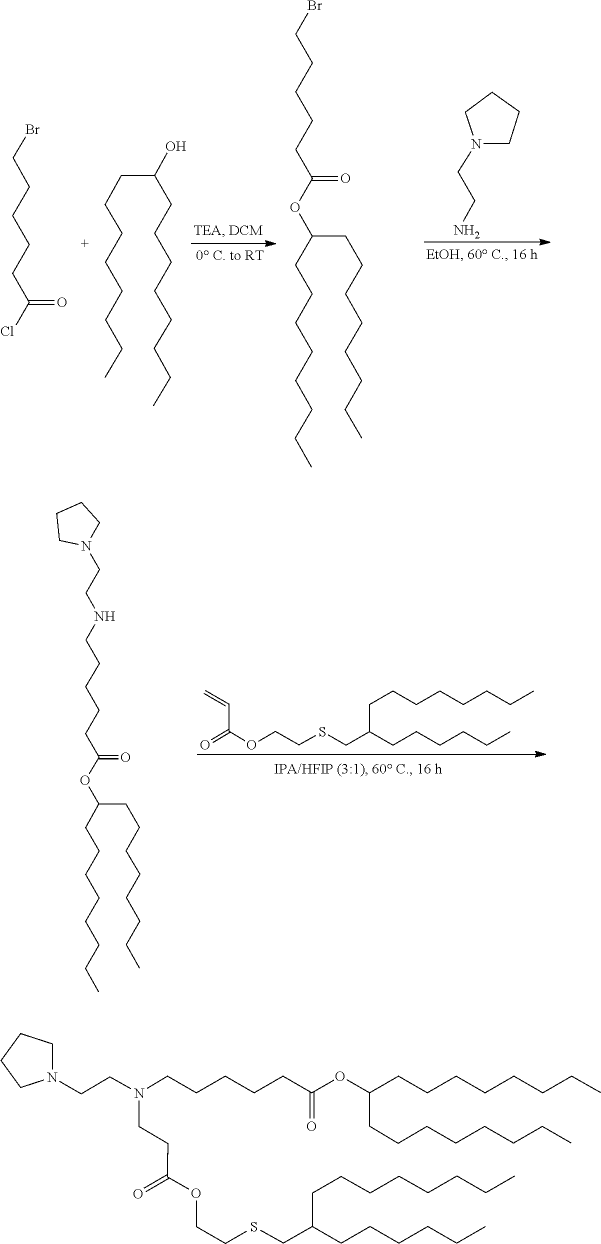

9-heptadecanol (2.56 g, 10 mmol) and TEA (1.21 g, 12 mmol) was mixed in anhydrous DCM (20 mL) and cooled to 0° C. 6-Bromohexanoyl chloride (2.14 g, 10 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 9-heptadecyl 6-bromohexanoate as colorless liquid (3.68 g, 85%).

A solution of afford 9-heptadecyl 6-bromohexanoate (1.72 g, 4 mmol) and 2-pyrrolidinoethylamine (4.56 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 9-heptadecyl 6-((2-pyrrolidinoethyl)amino)hexanoate as colorless oil (1.32 g, 71%).

A solution of 9-heptadecyl 6-((2-pyrrolidinoethyl)amino)hexanoate (93 mg, 0.2 mmol) and 2-((2-hexyldecyl)thio)ethyl acrylate (52 mg, 0.2 mmol) in a mixture of isopropanol/hexafluoroisopropanol (v:v, 3:1, 2 mL) was stirred at 60° C. for 16 hours. The solvents were removed to afford the crude product. ¹H NMR (500 MHz, CDCl₃) δ 4.85 (pent, J=6.3 Hz, 1H), 4.19 (t, J=7.1 Hz, 2H), 2.80 (t, J=7.3 Hz, 2H), 2.69 (t, J=7.1 Hz, 2H), 2.62-2.50 (m, 8H), 2.44 (dt, J=11.7, 7.5 Hz, 4H), 2.27 (t, J=7.6 Hz, 2H), 1.79 (s, 4H), 1.61 (pent, J=7.6 Hz, 2H), 1.48 (tdd, J=17.3, 13.6, 7.0 Hz, 7H), 1.40-1.16 (m, 52H), 0.89-0.84 (m, 12H).

### Example 26

### Synthesis of Compound 245

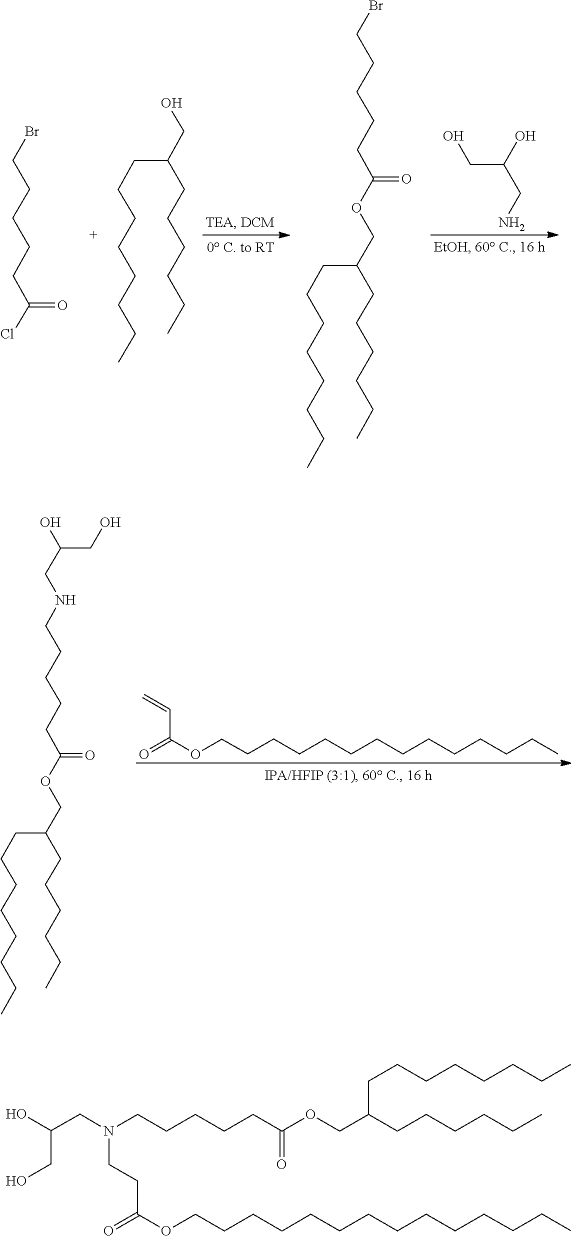

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 20.6 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%)

A solution of 2-hexyldecyl 6-bromohexanoate (1.68 g, 4 mmol) and 3-aminopropane-1,2-diol (3.64 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2,3-dihydroxypropyl)amino)hexanoate as colorless oil (1.2 g, 70%).

A solution of 2-hexyldecyl 6-((2,3-dihydroxypropyl)amino)hexanoate (0.430 mg, 1 mmol) and tetradecyl acrylate (0.268 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of NH₄OH, 5% MeOH in DCM) to obtain the product (0.352 g, 50%). ¹H NMR (500 MHz, CDCl₃) δ=4.07 (t, J=6.8 Hz, 2H), 3.96 (d, J=5.7 Hz, 2H), 3.78-3.70 (m, 2H), 3.50-03.42 (m, 1H), 2.92 (dt, J=14.0, 7.4 Hz, 1H), 2.71 (dt, J=12.9, 6.1 Hz, 1H), 2.54 (ddd, J=37.1, 14.3, 8.6 Hz, 3H), 2.47-2.38 (m, 4H), 2.30 (t, J=7.4 Hz, 2H), 1.69-1.55 (m, 5H), 1.44 (dd, J=16.5, 8.7 Hz, 2H), 1.26 (dd, J=9.5, 4.6 Hz, 49H), 0.87 (t, J=6.7 Hz, 9H).

### Example 27

### Synthesis of Compound 247

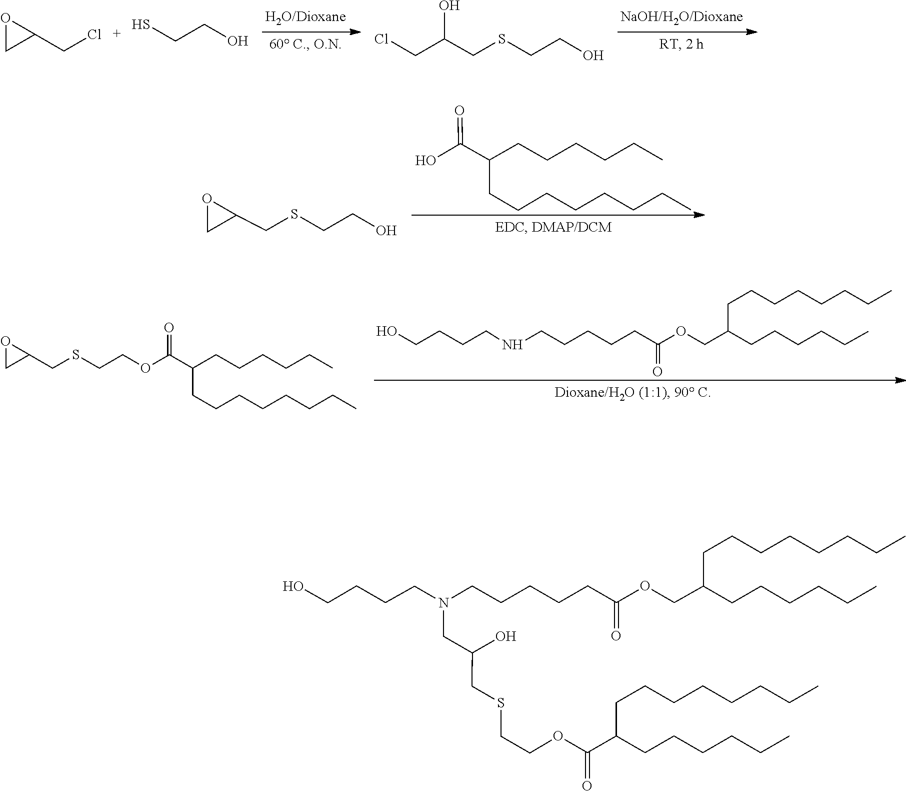

(±)-epichlorohydrin (2.04, 22 mmol) and 2-mercaptoethanol 1 (1.56 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature, then extracted with DCM (50 mL×3). DCM layers were combined, dried over anhydrous Na₂SO₄, and DCM was removed under vacuum to afford the crude product as colorless oil (1.82 g, 70%). The crude 2-(((oxiran-2-yl)methyl)thio)ethanol was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 2-(((oxiran-2-yl)methyl)thio)ethanol (1.34 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford the product as colorless oil (2.32 g, 62%). The crude 2-((oxiran-2-ylmethyl)thio)ethyl 2-hexyldecanoate was used without further purification.

2-((oxiran-2-ylmethyl)thio)ethyl 2-hexyldecanoate (0.372 g, 1 mmol) and 2-hexyldecyl 6-((4-hydroxybutyl)amino)hexanoate (0.428 g, 1 mmol) in a mixture of 1,4-dioxane and H₂O (v:v, 1:1, 2 mL) was heated to 90° C. and stirred for 24 hours. The solvent was removed under vacuum, and the residue was purified by silica-gel chromatography (5% MeOH in DCM with 0.1% ammonia hydroxide) to afford the product as colorless oil (0.482 g, 60%). ¹H NMR (500 MHz, CDCl₃) δ=4.23 (td, J=7.0, 2.3 Hz, 2H), 3.96 (d, J=5.7 Hz, 2H), 3.83 (q, J=6.2 Hz, 1H), 3.60 (dq, J=18.2, 6.7 Hz, 2H), 2.81 (t, J=7.0 Hz, 2H), 2.63 (d, J=5.9 Hz, 2H), 2.54 (dq, J=12.5, 6.3 Hz, 2H), 2.50-2.36 (m, 4H), 2.35-226 (m, 3H), 1.62 (ddq, J=21.7, 15.0, 7.1 Hz, 10H), 1.53-1.46 (m, 2H), 1.42 (q, J=6.6 Hz, 2H), 1.25 (t J=5.6 Hz, 47H), 0.87 (td, J=6.9, 3.8 Hz, 12H).

### Example 28

### Synthesis of Compound 249

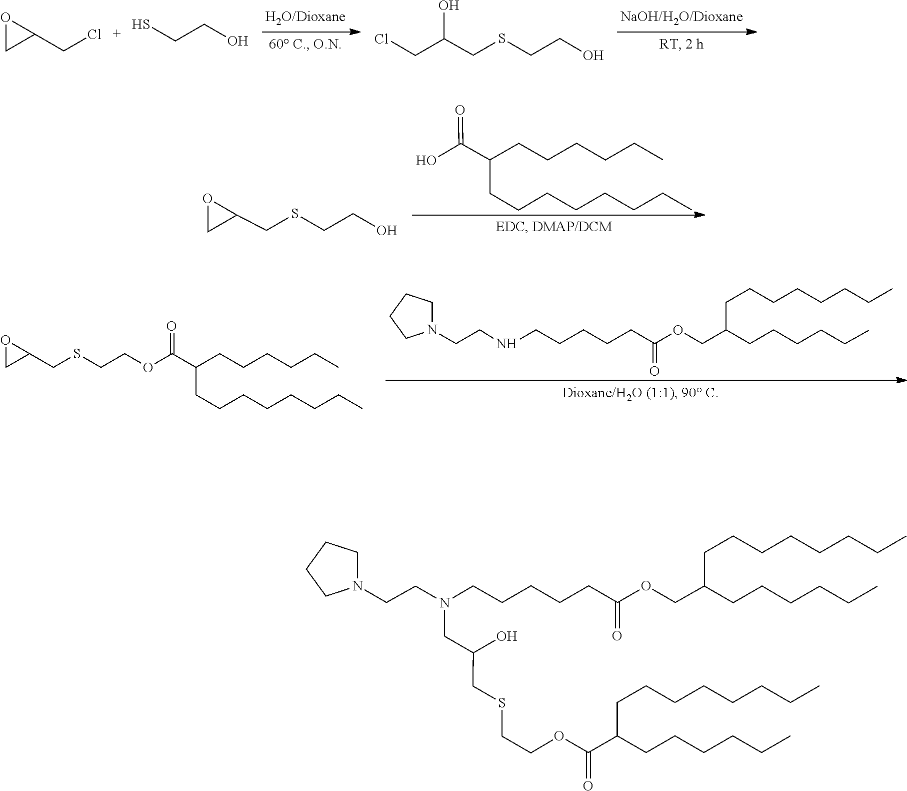

(±)-epichlorohydrin (2.04, 22 mmol) and 2-mercaptoethanol 1 (1.56 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature, then extracted with DCM (50 mL×3). DCM layers were combined, dried over anhydrous Na₂SO₄, and DCM was removed under vacuum to afford the crude product as colorless oil (1.82 g, 70%). The crude 2-(((oxiran-2-yl)methyl)thio)ethanol was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 2-(((oxiran-2-yl)methyl)thio)ethanol (1.34 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford the product as colorless oil (2.32 g, 62%). The crude 2-((oxiran-2-ylmethyl)thio)ethyl 2-hexyldecanoate was used without further purification. 2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-pyrrolidinoethylamine (4.56 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-pyrrolidinoethyl)amino)hexanoate as colorless oil (1.22 g, 67%). 2-((oxiran-2-ylmethyl)thio)ethyl 2-hexyldecanoate (74 mg, 0.2 mmol) and 2-hexyldecyl 6-((2-pyrrolidinoethyl)amino)hexanoate (90 mg, 0.2 mmol) in a mixture of 1,4-dioxane and H₂O (v:v, 1:1, 2 mL) was heated to 90° C. and stirred for 24 hours. The solvents were removed under vacuum to afford the crude product as colorless oil.

### Example 29

### Synthesis of Compound 250

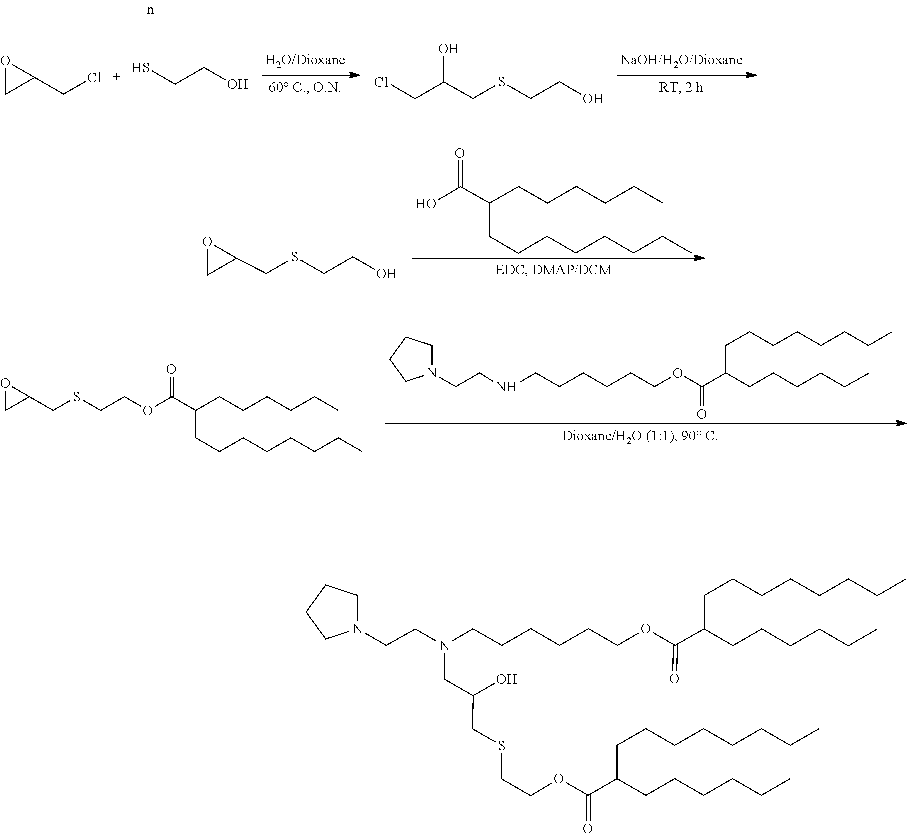

(±)-epichlorohydrin (2.04, 22 mmol) and 2-mercaptoethanol 1 (1.56 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature, then extracted with DCM (50 mL×3). DCM layers were combined, dried over anhydrous Na₂SO₄, and DCM was removed under vacuum to afford the crude product as colorless oil (1.82 g, 70%). The crude 2-(((oxiran-2-yl)methyl)thio)ethanol was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 2-(((oxiran-2-yl)methyl)thio)ethanol (1.34 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford the product as colorless oil (2.32 g, 62%). The crude 2-((oxiran-2-ylmethyl)thio)ethyl 2-hexyldecanoate was used without further purification.

2-Hexyl-1-decanoic acid (3.84 g, 15 mmol) and EDC-HCl (3.74 g, 19.5 mmol) and DMAP (2.38 g, 19.5 mmol) was stirred in anhydrous DCM for 20 minutes at room temperature. 6-Bromo-1-hexanol (2.72 g, 15 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL). The hexane layer was separated, dried over anhydrous Na₂SO₄, and evaporated under vacuum. The residue was purified by silica-gel chromatography (3% ethyl acetate in hexane) to afford 6-bromohexyl 2-hexyldecanoate as colorless oil (2.58 g, 41%).

A solution of 6-bromohexyl 2-hexyldecanoate (2.09 g, 5 mmol) and 2-pyrrolidinoethylamine (5.70 g, 50 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 6-((2-pyrrolidinoethyl)amino)hexyl 2-hexyldecanoate as colorless oil (1.61 g, 71%). 2-((oxiran-2-ylmethyl)thio)ethyl 2-hexyldecanoate (74 mg, 0.2 mmol) and 6-((2-pyrrolidinoethyl)amino)hexyl 2-hexyldecanoate (90 mg, 0.2 mmol) in a mixture of 1,4-dioxane and H₂O (v:v, 1:1, 2 mL) was heated to 90° C. and stirred for 24 hours. solvents were removed under vacuum to afford the crude product as colorless oil.

### Example 30

### Synthesis of Compound 251

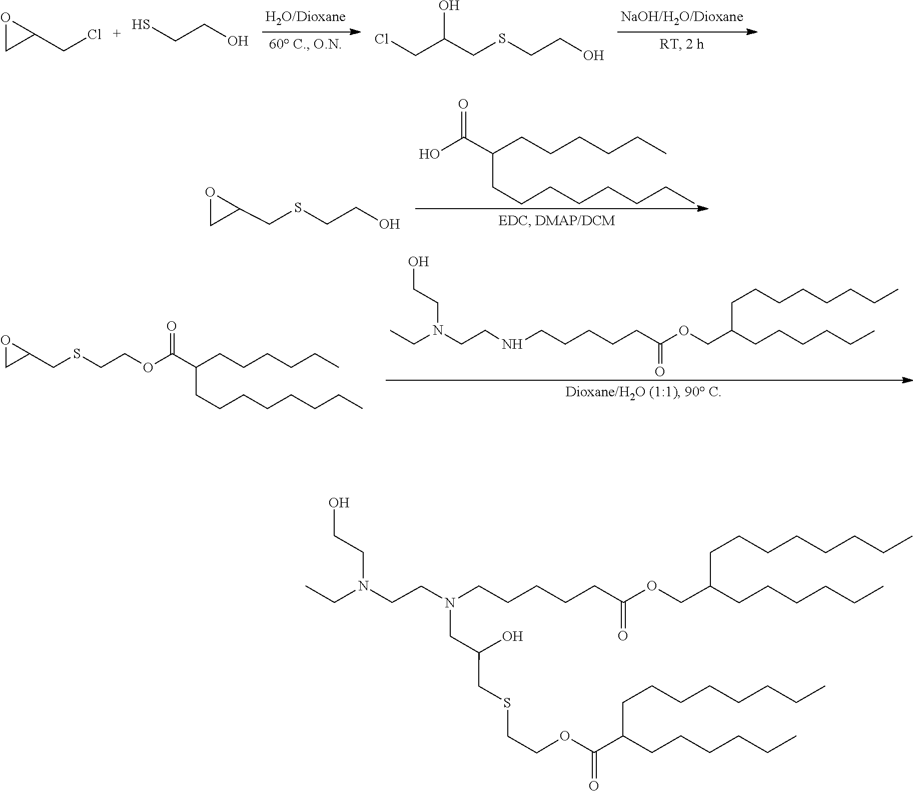

(±)-epichlorohydrin (2.04, 22 mmol) and 2-mercaptoethanol 1 (1.56 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature, then extracted with DCM (50 mL×3). DCM layers were combined, dried over anhydrous Na₂SO₄, and DCM was removed under vacuum to afford the crude product as colorless oil (1.82 g, 70%). The crude 2-(((oxiran-2-yl)methyl)thio)ethanol was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 2-(((oxiran-2-yl)methyl)thio)ethanol (1.34 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford the product as colorless oil (2.32 g, 62%). The crude 2-((oxiran-2-ylmethyl)thio)ethyl 2-hexyldecanoate was used without further purification.

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and 2-[(2-aminoethyl)(ethyl)amino]ethan-1-ol (4.22 g, 32 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((2-hydroxyethyl)(ethyl)aminoethyl)amino)hexanoate as colorless oil (1.18 g, 63%).

2-((oxiran-2-ylmethyl)thio)ethyl 2-hexyldecanoate (74 mg, 0.2 mmol) and 2-hexyldecyl 6-((2-hydroxyethyl)(ethyl)aminoethyl)amino)hexanoate (94 mg, 0.2 mmol) in a mixture of 1,4-dioxane and H₂O (v:v, 1:1, 2 mL) was heated to 90° C. and stirred for 24 hours. The solvents were removed under vacuum to afford the crude product as colorless oil.

### Example 31

### Synthesis of Compound 253

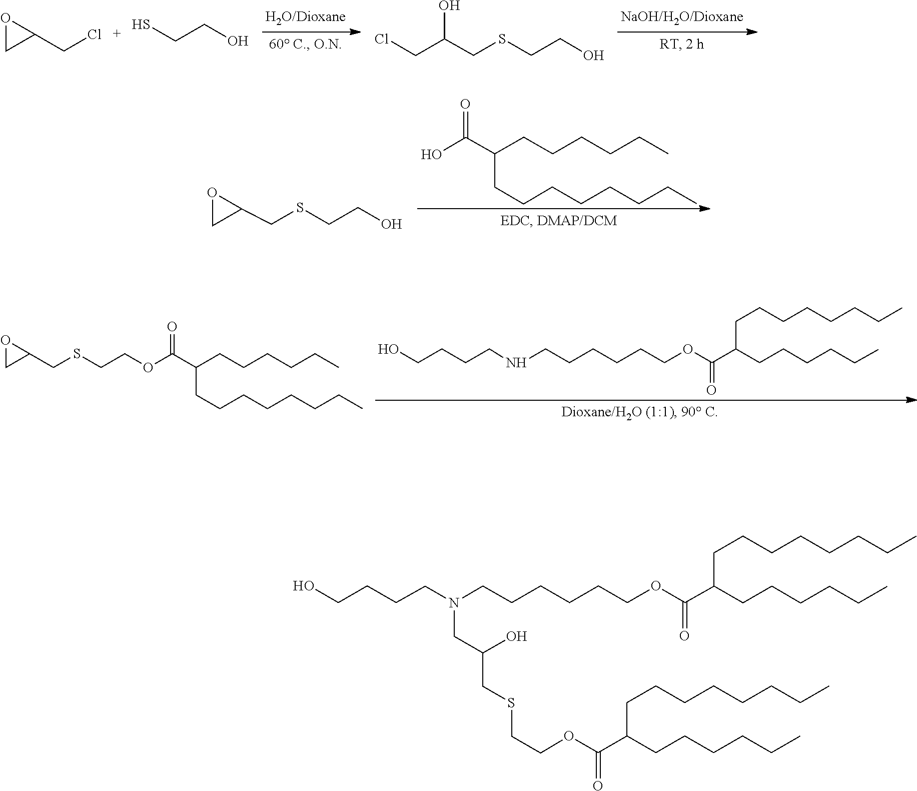

(±)-epichlorohydrin (2.04, 22 mmol) and 2-mercaptoethanol 1 (1.56 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature, then extracted with DCM (50 mL×3). DCM layers were combined, dried over anhydrous Na₂SO₄, and DCM was removed under vacuum to afford the crude product as colorless oil (1.82 g, 70%). The crude 2-(((oxiran-2-yl)methyl)thio)ethanol was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 2-(((oxiran-2-yl)methyl)thio)ethanol (1.34 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford the product as colorless oil (2.32 g, 62%). The crude 2-((oxiran-2-ylmethyl)thio)ethyl 2-hexyldecanoate was used without further purification.

2-((oxiran-2-ylmethyl)thio)ethyl 2-hexyldecanoate (75 mg, 0.2 mmol) and 6-((4-hydroxybutyl)amino)hexyl 2-hexyldecanoate (86 mg, 0.2 mmol) in a mixture of 1,4-dioxane and H₂O (v:v, 1:1, 1 mL) was heated to 90° C. and stirred for 24 hours. The solvents were removed under vacuum and the residue was purified by silica-gel chromatography (5% MeOH in DCM with 0.1% ammonia hydroxide) to afford the product as light brown oil (205 mg, 51%). ¹H NMR (500 MHz, CDCl₃) δ 4.23 (td, J=7.0, 2.6 Hz, 2H), 4.14-4.08 (m, 1H), 4.05 (t, J=6.7 Hz, 2H), 3.82 (q, J=6.1 Hz, 1H), 3.61 (td, J=11.4, 5.0 Hz, 2H), 2.81 (td, J=6.8, 1.5 Hz, 2H), 2.68-2.59 (m, 2H), 2.59-2.50 (m, 2H), 2.50-2.35 (m, 4H), 2.30 (tt, J=9.4, 5.3 Hz, 2H), 1.68-1.51 (m, 10H), 1.52-1.38 (m, 6H), 1.35 (q, J=7.1 Hz, 3H), 1.32-1.15 (m, 42H), 0.86 (t, J=6.7 Hz, 12H).

### Example 32

### Synthesis of Compound 254

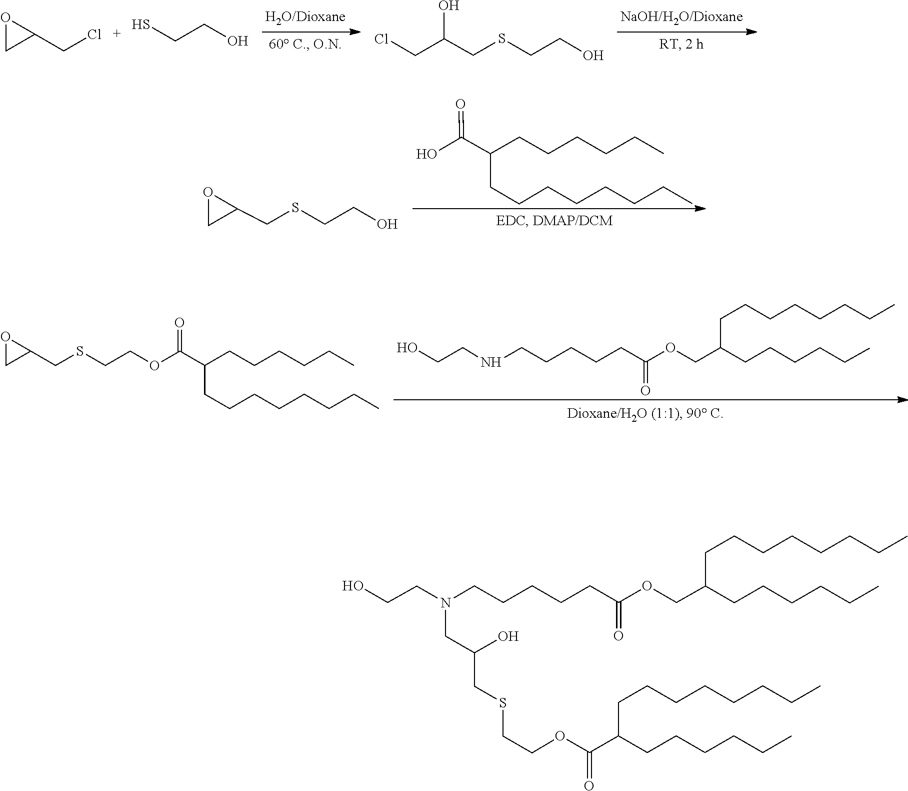

(±)-epichlorohydrin (2.04, 22 mmol) and 2-mercaptoethanol 1 (1.56 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature, then extracted with DCM (50 mL×3). DCM layers were combined, dried over anhydrous Na₂SO₄, and DCM was removed under vacuum to afford the crude product as colorless oil (1.82 g, 70%). The crude 2-(((oxiran-2-yl)methyl)thio)ethanol was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 2-(((oxiran-2-yl)methyl)thio)ethanol (1.34 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford the product as colorless oil (2.32 g, 62%). The crude 2-((oxiran-2-ylmethyl)thio)ethyl 2-hexyldecanoate was used without further purification.

2-((oxiran-2-ylmethyl)thio)ethyl 2-hexyldecanoate (75 mg, 0.2 mmol) and 2-hexyldecyl 6-((2-hydroxyethyl)amino)hexanoate (80 mg, 0.2 mmol) in a mixture of 1,4-dioxane and H₂O (v:v, 1:1, 1 mL) was heated to 90° C. and stirred for 24 hours. The solvents were removed under vacuum and the residue was purified by silica-gel chromatography (5% MeOH in DCM with 0.1% ammonia hydroxide) to afford the product as light brown oil (189 mg, 48%). ¹H NMR (500 MHz, CDCl₃) δ 4.28-4.18 (m, 2H), 3.96 (dd, J=5.8, 1.4 Hz, 2H), 3.81 (q, J=6.0 Hz, 1H), 3.74 (td, J=5.7, 1.6 Hz, 2H), 3.39-3.10 (m, 1H), 2.80 (td, J=6.9, 1.5 Hz, 2H), 2.76-2.65 (m, 2H), 2.63-2.55 (m, 2H), 2.55-2.38 (m, 3H), 2.37-2.24 (m, 3H), 1.65 (dddq, J=35.3, 20.3, 14.3, 7.3 Hz, 7H), 1.53-1.36 (m, 4H), 1.26 (dd, J=10.9, 5.2 Hz, 48H), 0.93-0.80 (m, 12H).

### Example 33

### Synthesis of Compound 255

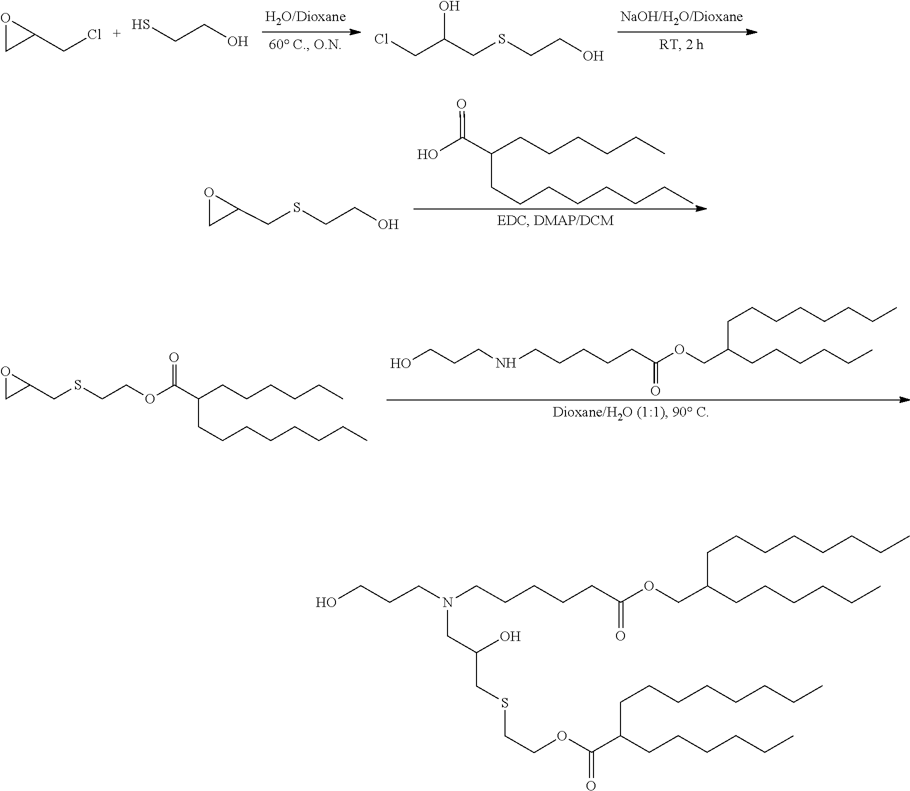

(±)-epichlorohydrin (2.04, 22 mmol) and 2-mercaptoethanol 1 (1.56 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature, then extracted with DCM (50 mL×3). DCM layers were combined, dried over anhydrous Na₂SO₄, and DCM was removed under vacuum to afford the crude product as colorless oil (1.82 g, 70%). The crude 2-(((oxiran-2-yl)methyl)thio)ethanol was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 2-(((oxiran-2-yl)methyl)thio)ethanol (1.34 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford the product as colorless oil (2.32 g, 62%). The crude 2-((oxiran-2-ylmethyl)thio)ethyl 2-hexyldecanoate was used without further purification.

2-((oxiran-2-ylmethyl)thio)ethyl 2-hexyldecanoate (75 mg, 0.2 mmol) and 2-hexyldecyl 6-((3-hydroxypropyl)amino)hexanoate (82 mg, 0.2 mmol) in a mixture of 1,4-dioxane and H₂O (v:v, 1:1, 1 mL) was heated to 90° C. and stirred for 24 hours. The solvents were removed under vacuum to afford the crude product as colorless oil.

### Example 34

### Synthesis of Compound 256

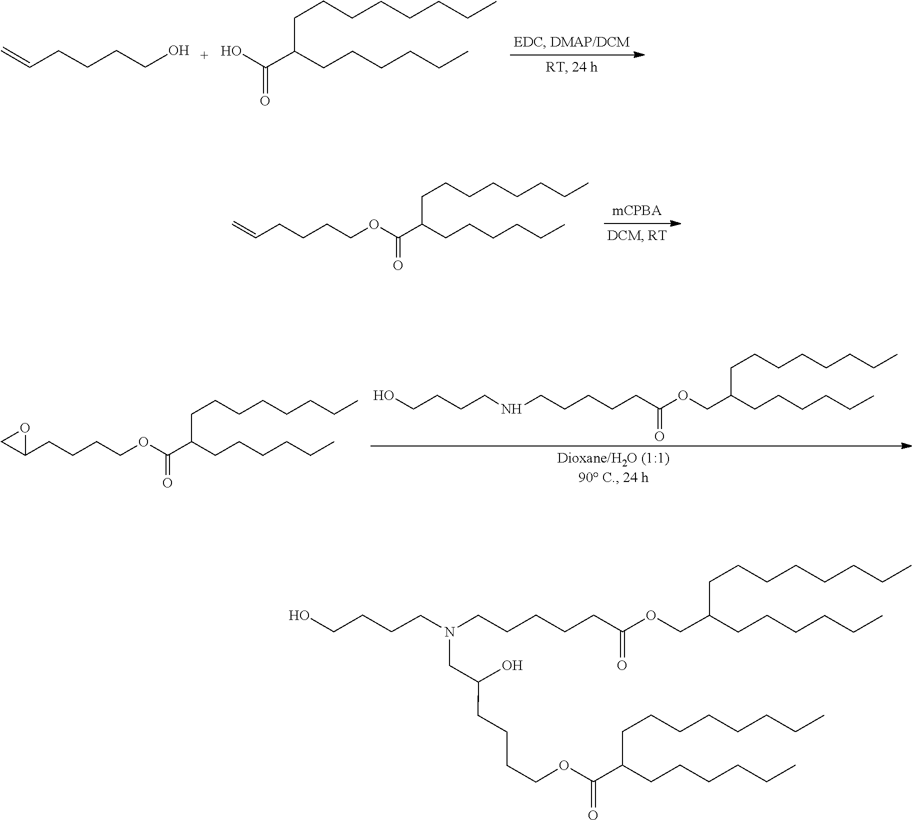

2-Hexyldecanoic acid (5.12 g, 20 mmol), EDC-HCl (4.97 g, 26 mmol) and DMAP (3.17 g, 26 mmol) was stirred in anhydrous DCM for 20 minutes at room temperature. 5-Hexen-1-ol (2.0 g, 20 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (20 mL×2). The hexane layer was separated, dried over anhydrous Na₂SO₄, and evaporated under vacuum to afford hex-5-en-1-yl 2-hexyldecanoate as colorless oil (4.21 g, 62%). The product was used without further purification.

To a solution of ester (10 mmol) in anhydrous DCM was added mCPBA (2.51 g, 11 mmol). The resulting mixture was stirred at room temperature overnight. DCM was removed under vacuum. 100 mL of hexane was added to the residue, and the mixture was washed with saturated Na₂S₂O₃, saturated Na₂CO₃, brine, dried over anhydrous Na₂SO₄. The solvent was removed under vacuum to afford 4-(oxiran-2-yl)butyl 2-hexyldecanoate as colorless oil (2.12 g, 59%). The crude product was used without further purification.

4-(oxiran-2-yl)butyl 2-hexyldecanoate (71 mg, 0.2 mmol) and 2-hexyldecyl 6-((4-hydroxybutyl)amino)hexanoate (86 mg, 0.2 mmol) in a mixture of 1,4-dioxane and H₂O (v:v, 1:1, 1 mL) was heated to 90° C. and stirred for 24 hours. The solvents were removed under vacuum and the residue was purified by silica-gel chromatography (5% MeOH in DCM with 0.1% ammonia hydroxide) to afford the product as light brown oil (175 mg, 44%). ¹H NMR (500 MHz, CDCl₃) δ 4.06 (q, J=6.0 Hz, 2H), 3.95 (d, J=5.7 Hz, 2H), 3.73-3.53 (m, 3H), 2.56 (dd, J=17.0, 11.6 Hz, 2H), 2.47-2.33 (m, 3H), 2.29 (t, J=7.4 Hz, 3H), 1.60 (dh, J=30.9, 7.6 Hz, 13H), 1.51-1.33 (m, 7H), 1.26 (dd, J=11.8, 6.5 Hz, 48H), 0.86 (dtd, J=7.1, 4.2, 2.0 Hz, 12H).

### Example 35

### Synthesis of Compound 257

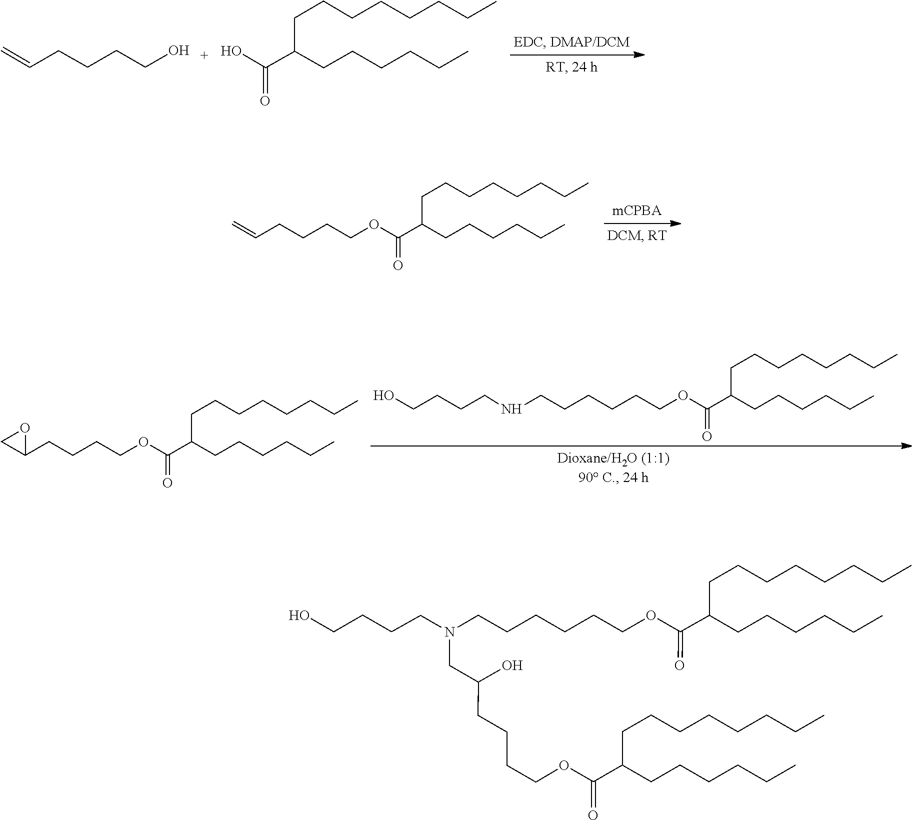

2-Hexyldecanoic acid (5.12 g, 20 mmol), EDC-HCl (4.97 g, 26 mmol) and DMAP (3.17 g, 26 mmol) was stirred in anhydrous DCM for 20 minutes at room temperature. 5-Hexen-1-ol (2.0 g, 20 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (20 mL×2). The hexane layer was separated, dried over anhydrous Na₂SO₄, and evaporated under vacuum to afford hex-5-en-1-yl 2-hexyldecanoate as colorless oil (4.21 g, 62%). The product was used without further purification.

To a solution of ester (10 mmol) in anhydrous DCM was added mCPBA (2.51 g, 11 mmol).

The resulting mixture was stirred at room temperature overnight. DCM was removed under vacuum. 100 mL of hexane was added to the residue, and the mixture was washed with saturated Na₂S₂O₃, saturated Na₂CO₃, brine, dried over anhydrous Na₂SO₄. The solvent was removed under vacuum to afford 4-(oxiran-2-yl)butyl 2-hexyldecanoate as colorless oil (2.12 g, 59%). The crude product was used without further purification. 4-(oxiran-2-yl)butyl 2-hexyldecanoate (71 mg, 0.2 mmol) and 6-((4-hydroxybutyl)amino)hexyl 2-hexyldecanoate (85.5 g, 0.2 mmol) in a mixture of 1,4-dioxane and H₂O (v:v, 1:1, 1 mL) was heated to 90° C. and stirred for 24 hours. The solvents were removed under vacuum to afford the product as light brown oil.

### Example 36

### Synthesis of Compound 297

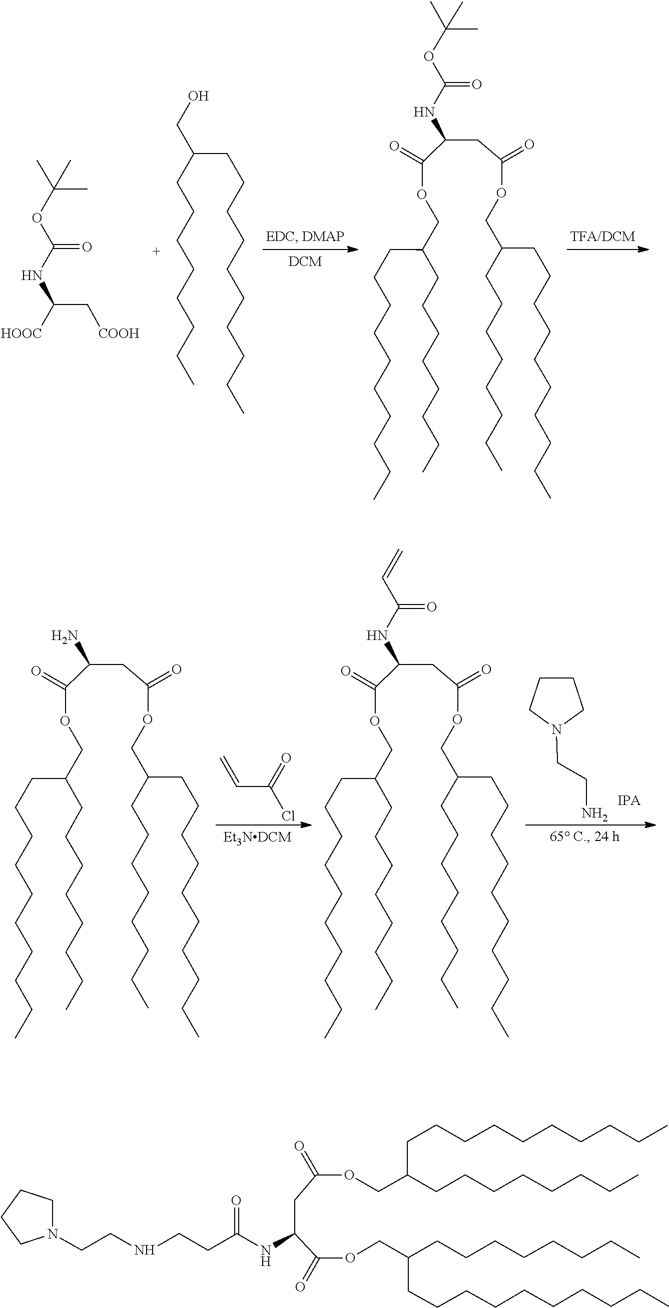

Boc-L-aspartic acid (4.60 g, 20 mmol), EDC-HCl (9.2 g, 48 mmol) and DMAP (5.91 g, 48 mmol) in anhydrous DCM (50 mL) was stirred at room temperature for 20 minutes. 2-octyldodecan-1-ol (11.90 g, 40 mmol) was added, and the resulting mixture was stirred for 48 hours at room temperature. DCM was removed, the residue was re-suspended in H₂O (100 mL) and extracted with hexane (50 mL×3). The combined hexane layers were washed with acetonitrile (50 mL×2) and brine (50 mL×2), dried over Na₂SO₄. The mixture was filtered, and solvent was removed under vacuum to obtain (2S)-bis(2-octyldodecyl) 2-((tert-butoxycarbonyl)amino)succinate as light-yellow oil (10.2 g, 64%).

(2S)-bis(2-octyldodecyl) 2-((tert-butoxycarbonyl)amino)succinate (10.2 g, 12.8 mmol) in a mixture of trifluoroacetic acid and dichloromethane (v:v, 1:1, 20 mL) was stirred at room temperature for 3 hours. The pH of the reaction mixture was adjusted to 11 (monitored using pH paper) with 20% of NaOH solution. 50 mL of saturated NaHCO₃ was added to the mixture, which was then extracted with dichloromethane (50 mL×4). Dichloromethane layers were combined, washed with H₂O (50 mL×2) and brine (50 mL×2), dried over Na₂SO₄. The mixture was filtered, and the solvent removed under vacuum to obtain (2S)-bis(2-octyldodecyl) 2-((tert-butoxycarbonyl)amino)succinate as light-yellow oil (8.2 g, 92%). (2S)-bis(2-octyldodecyl) 2-((tert-butoxycarbonyl)amino)succinate (3.47 g, 5 mmol) and triethylamine (0.697 g, 6 mmol) in anhydrous DCM (30 mL) was cooled to 0° C. Acryloyl chloride (0.542 g, 6 mmol) was slowly added into the resulting mixture. The reaction mixture was stirred at 0° C. for 30 minutes and then room temperature for 3 hours. DCM was evaporated under vacuum, and the residue was taken-up in hexane (150 mL). The hexane suspension was washed with H₂O (50 mL×2), brine (50 mL×2) and acetonitrile (20 mL×2). The hexane layer was separated, dried over anhydrous Na₂SO₄, and evaporated under vacuum to afford (2S)-bis(2-octyldodecyl) 2-acrylamidosuccinate as light-brown oil (2.45 g, 65%)

A solution of (2S)-bis(2-octyldodecyl) 2-acrylamidosuccinate (0.745 mg, 1 mmol) and 2-(pyrrolidin-1-yl)ethanamine (0.114 g, 1 mmol) in a mixture of isopropanol (2 mL) was stirred at 65° C. for 24 hours. The solvents were removed to afford the product as colorless oil (421 mg, 49%).

### Example 37

### Synthesis of Compound 341

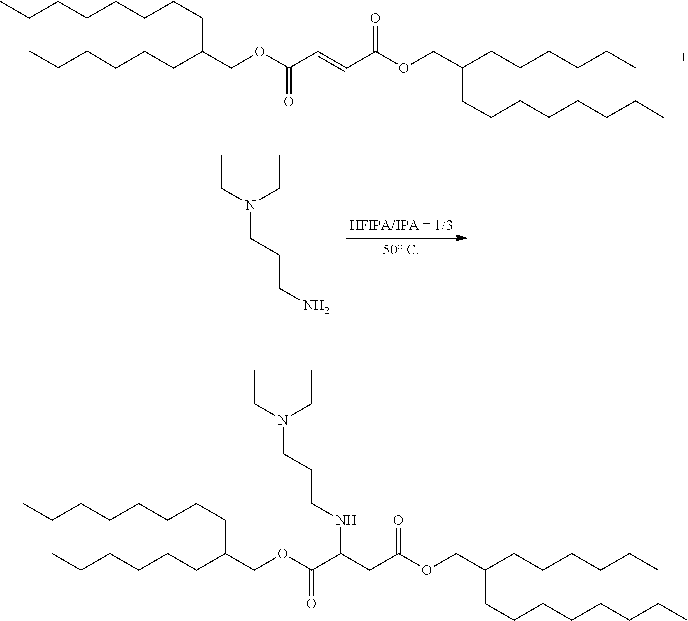

4.8 g 2-hexyl-1-decanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC when the starting materials were consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane to yield the di(2-hexyldecyl) fumarate (85% yield, 4.8 g). 2.0 g di(2-hexyldecyl) fumarate (1 eq.) and 690 mg 3-(diethylamino)propylamine (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation to yield a yellow oily residue as the crude product. The crude product was purified by silica column chromatography, with a mobile phase composed of 0% to 33% ethyl acetate in hexane. The product was collected as a pale-yellow oil (40% yield, 980 mg). ¹H NMR (500 MHz, CDCl₃) δ 3.99 (ddd, J=27.6, 6.2, 2.9 Hz, 4H), 3.60 (dt, J=6.5, 3.8 Hz, 1H), 2.74-2.57 (m, 3H), 2.48 (ddtd, J=21.7, 17.8, 9.1, 4.7 Hz, 7H), 1.90 (s, 1H), 1.59 (pent, J=7.6 Hz, 4H), 1.25 (d, J=7.3 Hz, 48H), 0.99 (ddd, J=8.3, 6.8, 2.4 Hz, 6H), 0.87 (td, J=7.2, 2.5 Hz, 12H).

### Example 38

### Synthesis of Compound 347

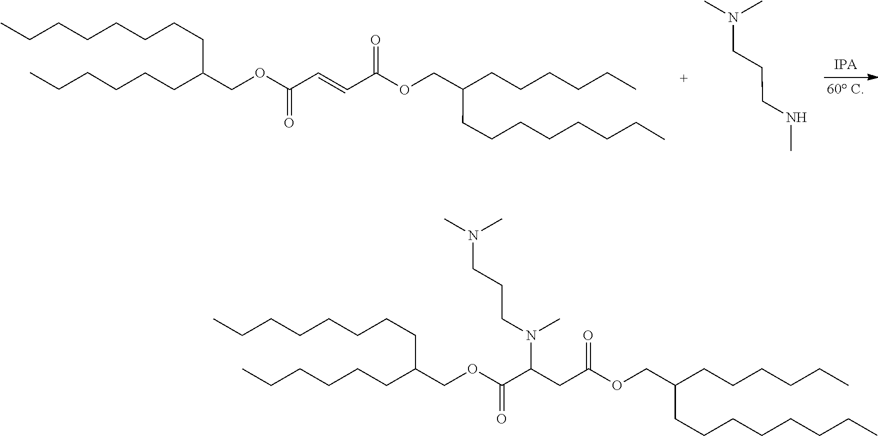

4.8 g 2-hexyl-1-decanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC when the starting materials were consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane to yield the di(2-hexyldecyl) fumarate (85% yield, 4.8 mg).

1.7 g di(2-hexyldecyl) fumarate (1 eq.) and 1.0 g N,N,N′-Trimethyl-1,3-propanediamine (3 eq) were mixed with 5 mL of IPA (isopropyl alcohol) in a 25 mL round bottom flask. The mixture was heated to 60° C. with an oil bath for ~20 h. The reaction was monitored by TLC and ended when the starting materials were consumed. Then the solvent was removed by vacuum distillation, to yield the yellow oily residue as the crude product. The crude product was purified by silica column chromatography, with a mobile phase composed of 0% to 33% ethyl acetate in hexane. The purified product was a pale-yellow oil (30% yield, 610 mg). ¹H NMR (500 MHz, CDCl₃) δ 4.06-3.92 (m, 4H), 3.80 (t, J=7.6 Hz, 1H), 2.86-2.78 (m, 1H), 2.62-2.54 (m, 2H), 2.49 (dt, J=12.4, 6.8 Hz, 1H), 2.29 (d, J=2.5 Hz, 3H), 2.24 (t, J=7.7 Hz, 2H), 2.21 (d, J=2.3 Hz, 6H), 1.60 (q, J=7.8 Hz, 4H), 1.27 (d, J=11.2 Hz, 48H), 0.87 (ddd, J=7.5, 4.8, 2.0 Hz, 12H).

### Example 39

### Synthesis of Compound 348

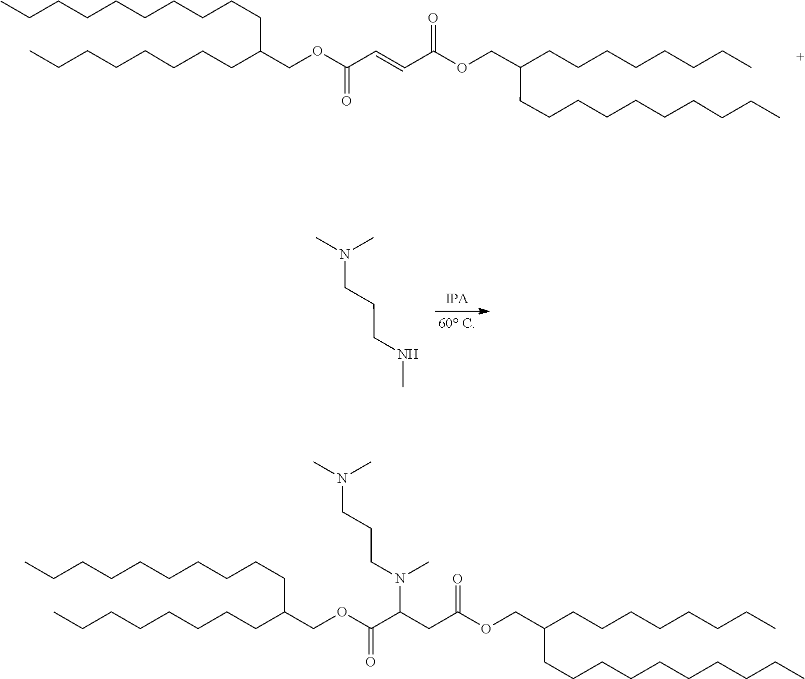

6.0 g 2-octyl-1-dodecanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC when the 2-octyl-1-dodecanol was consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane (85% yield, 5.8 g). 2.0 g di(2-octyldodecyl) fumarate (1 eq.) and 1.0 g N,N,N′-Trimethyl-1,3-propanediamine (3 eq) were mixed with 5 mL of IPA (isopropyl alcohol) in a 25 mL round bottom flask. The mixture was heated to 60° C. with an oil bath for ~20 h. The reaction was monitored by TLC and ended when the starting materials were consumed. Then the solvent was removed by vacuum distillation, to yield the yellow oily residue as the crude product. The crude product was purified by silica column chromatography, with a mobile phase composed of 0% to 33% ethyl acetate in hexane. The product was a pale-yellow oil (30% yield, 700 mg). ¹H NMR (500 MHz, CDCl₃) δ 3.99 (dddd, J=22.8, 20.8, 10.9, 5.7 Hz, 4H), 3.80 (t, J=7.5 Hz, 1H), 2.82 (dd, J=15.9, 8.1 Hz, 1H), 2.62-2.53 (m, 2H), 2.49 (ddd, J=12.4, 7.9, 6.1 Hz, 1H), 2.29 (s, 3H), 2.22 (d, J=19.1 Hz, 8H), 1.67-1.55 (m, J=6.6 Hz, 4H), 1.25 (s, 64H), 0.88 (t, J=6.9 Hz, 12H).

### Example 40

### Synthesis of Compound 350

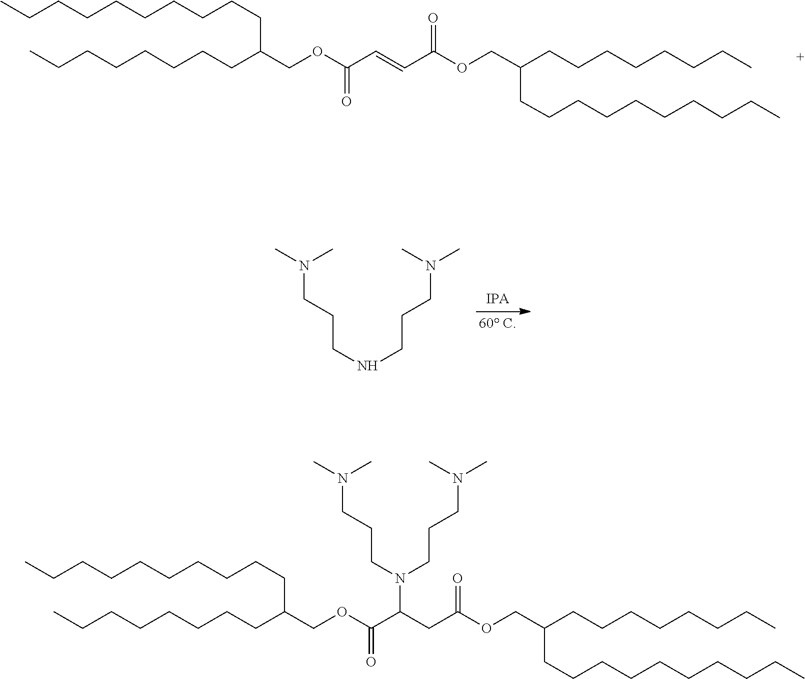

6.0 g 2-octyl-1-dodecanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC when the 2-octyl-1-dodecanol was consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane (85% yield, 5.8 g).

2.0 g di(2-octyldodecyl) fumarate (1 eq.) and 1.5 g N1,N3-Dimethyl-N1-(3-(methylamino)propyl)propane-1,3-diamine (3 eq) were mixed with 5 mL of IPA (isopropyl alcohol) in a 25 mL round bottom flask. The mixture was heated to 60° C. with an oil bath for ~20 h. The reaction was monitored by TLC and ended when the starting materials were consumed. Then the solvent was removed by vacuum distillation, to yield the yellow oily residue as the crude product. The crude product was purified by silica column chromatography, with a mobile phase composed of 0% to 33% ethyl acetate in hexane. The product was collected as a pale-yellow oil (30%, 770 mg). ¹H NMR (500 MHz, CDCl₃) δ 3.99 (ddt, J=16.3, 11.0, 5.6 Hz, 4H), 3.88 (dd, J=8.0, 6.7 Hz, 1H), 2.82 (dd, J=16.1, 8.0 Hz, 1H), 2.61-2.48 (m, 5H), 2.19 (s, 15H), 1.57 (dddd, J=27.7, 13.8, 8.8, 5.0 Hz, 7H), 1.26 (d, J=6.7 Hz, 64H), 0.87 (t, J=6.8 Hz, 12H).

### Example 41

### Synthesis of Compound 354

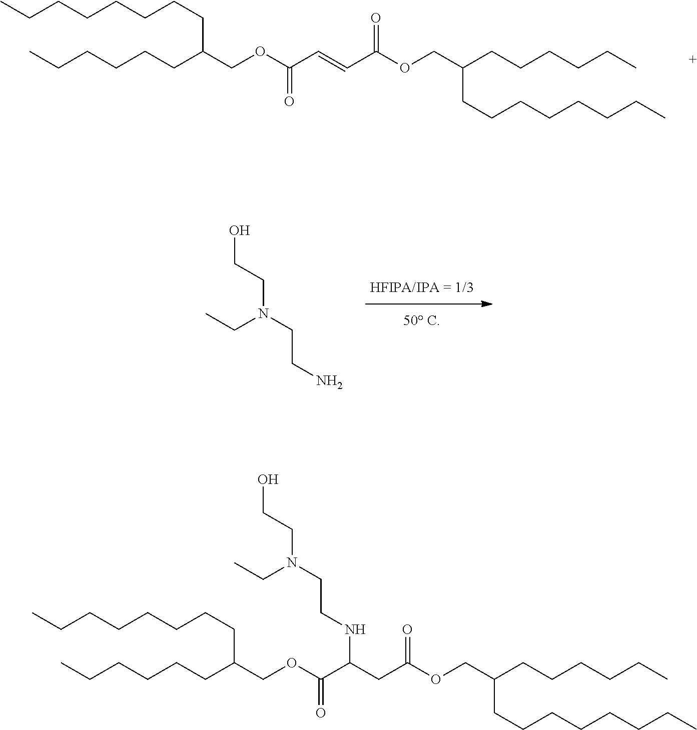

4.8 g 2-hexyl-1-decanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC when the starting materials were consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane to yield the di(2-hexyldecyl) fumarate (85% yield, 4.8 g).

Trifluoroacetate (7.1 g, 60 mmol) was added slowly added into a solution of 2-(2-aminoethylamino)ethanol (6.2 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO. The organic solvent was removed under vacuum to afford 2-[(2-aminoethyl)(ethyl)amino]ethan-1-ol as a colorless oil (2.4 g).

1.8 g di(2-hexyldecyl) fumarate (1 eq.) and 650 mg 2-[(2-aminoethyl)(ethyl)amino]ethan-1-ol (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 0% to 33% ethyl acetate in hexane. The product was collected as a pale-yellow oil (35% yield, 780 mg). ¹H NMR (500 MHz, CDCl₃) δ 4.01 (dd, J=27.6, 5.8 Hz, 4H), 3.64 (dd, J=7.1, 5.8 Hz, 1H), 3.54 (t, J=5.2 Hz, 2H), 2.81-2.50 (m, 11H), 1.61 (dd, J=13.9, 6.9 Hz, 2H), 1.26 (d, J=6.3 Hz, 49H), 1.01 (t, J=7.1 Hz, 3H), 0.87 (t, J=6.8 Hz, 12H).

### Example 42

### Synthesis of Compound 355

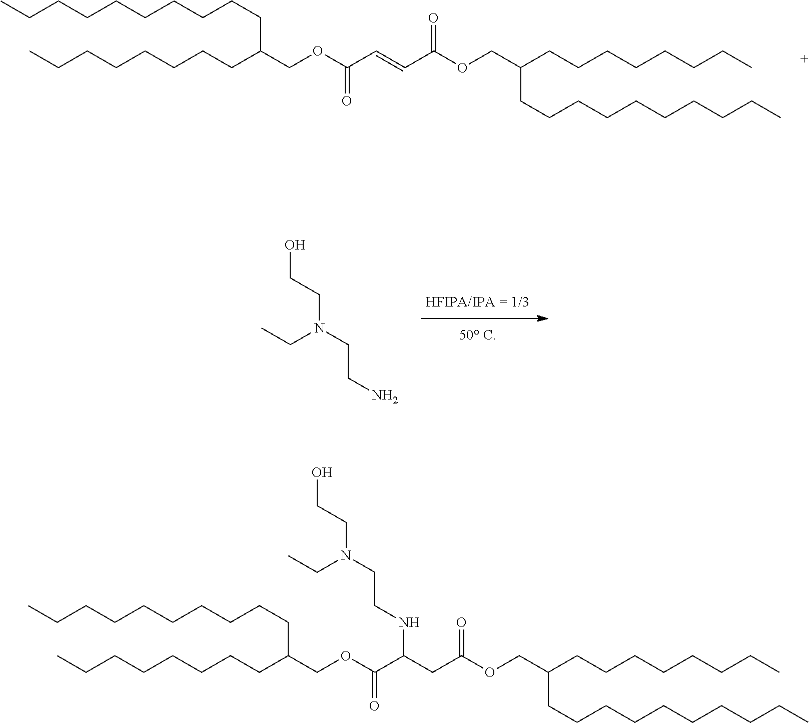

6.0 g 2-octyl-1-dodecanol (2 eq.) and 3.9 g diisopropyl ethyl amine eq were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC when the 2-octyl-1-dodecanol was consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane (85% yield, 5.8 g).

Trifluoroacetate (7.1 g, 60 mmol) was added slowly added into a solution of 2-(2-aminoethylamino)ethanol (6.2 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO. The organic solvent was removed under vacuum to afford 2-[(2-aminoethyl)(ethyl)amino]ethan-1-ol as a colorless oil (2.4 g).

2.0 g di(2-octyldodecyl) fumarate (1 eq.) and 650 mg 2-[(2-aminoethyl)(ethyl)amino]ethan-1-ol (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 0% to 33% ethyl acetate in hexane. The product was collected as a pale-yellow oil (35% yield, 840 mg). ¹H NMR (500 MHz, CDCl₃) δ 4.01 (dd, J=27.8, 5.8 Hz, 4H), 3.64 (dd, J=7.0, 5.8 Hz, 1H), 3.55 (t, J=5.1 Hz, 2H), 2.81-2.52 (m, 11H), 1.62 (s, 2H), 1.33-1.20 (m, 67H), 1.02 (t, J=7.1 Hz, 3H), 0.87 (t, J=6.9 Hz, 12H).

### Example 43

### Synthesis of Compound 356

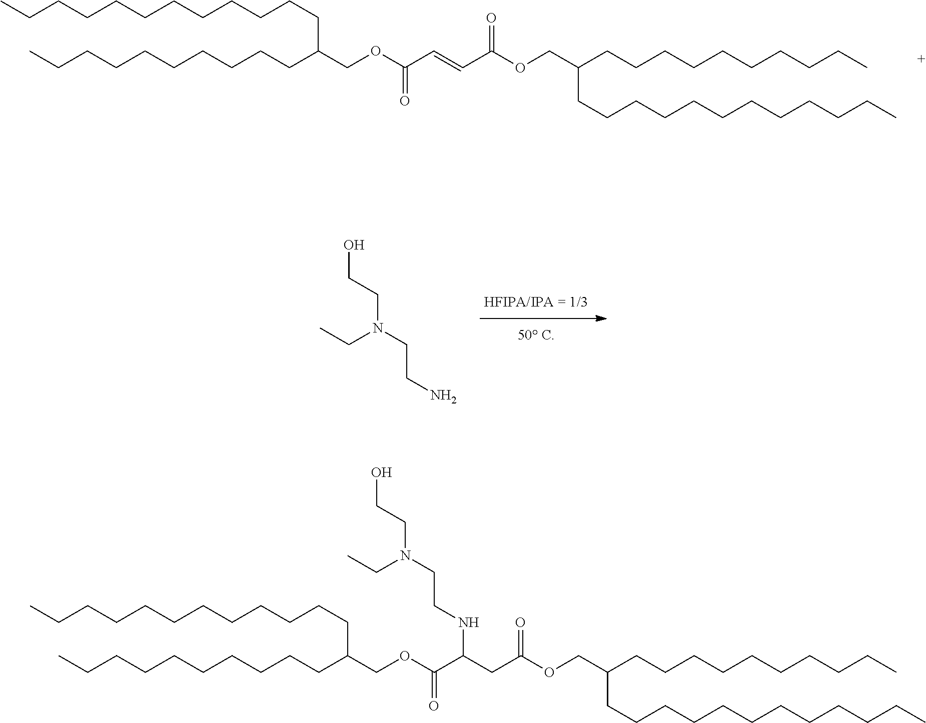

7.1 g 2-decyl-1-tetradecanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane (85%, 6.7 g).

Trifluoroacetate (7.1 g, 60 mmol) was added slowly added into a solution of 2-(2-aminoethylamino)ethanol (6.2 g, 60 mmol) in acetonitrile (30 mL) at 0° C. while stirring. After stirring for 3 hours, ethyl iodide (9.4 g, 60 mmol) and DIEA (8.5 g, 66 mmol) was added. The reaction mixture was heated to 40° C. and stirred overnight. The solvent was removed under vacuum. H₂O (100 mL) was added to the residue, and the aqueous solution was extracted with ethyl acetate (30 mL×5). The organic layers were combined, dried over anhydrous Na₂SO₄, and the solvent is removed under vacuum to afford a crude light-yellow oil. 10% NaOH solution (20 mL) was added to the above crude oil. The resulting solution was stirred at 60° C. for 2 hours. The reaction mixture was cooled to room temperature and extracted with DCM (30 mL×6). DCM layers were combined, washed with brine (20 mL), dried over anhydrous Na₂SO. The organic solvent was removed under vacuum to afford 2-[(2-aminoethyl)(ethyl)amino]ethan-1-ol as a colorless oil (2.4 g).

2.0 g di(2-decyltetradecyl) fumarate (1 eq.) and 500 mg 2-[(2-aminoethyl)(ethyl)amino]ethan-1-ol (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 0% to 33% ethyl acetate in hexane. The product was collected as a pale-yellow oil (35% yield, 820 mg). ¹H NMR (500 MHz, CDCl₃) δ 4.00 (dd, J=27.8, 5.8 Hz, 4H), 3.64 (dd, J=7.0, 5.9 Hz, 1H), 3.54 (t, J=5.1 Hz, 2H), 2.82-2.45 (m, 12H), 1.62 (s, 2H), 1.25 (s, 82H), 1.01 (t, J=7.1 Hz, 3H), 0.87 (t, J=6.9 Hz, 12H).

### Example 44

### Synthesis of Compound 357

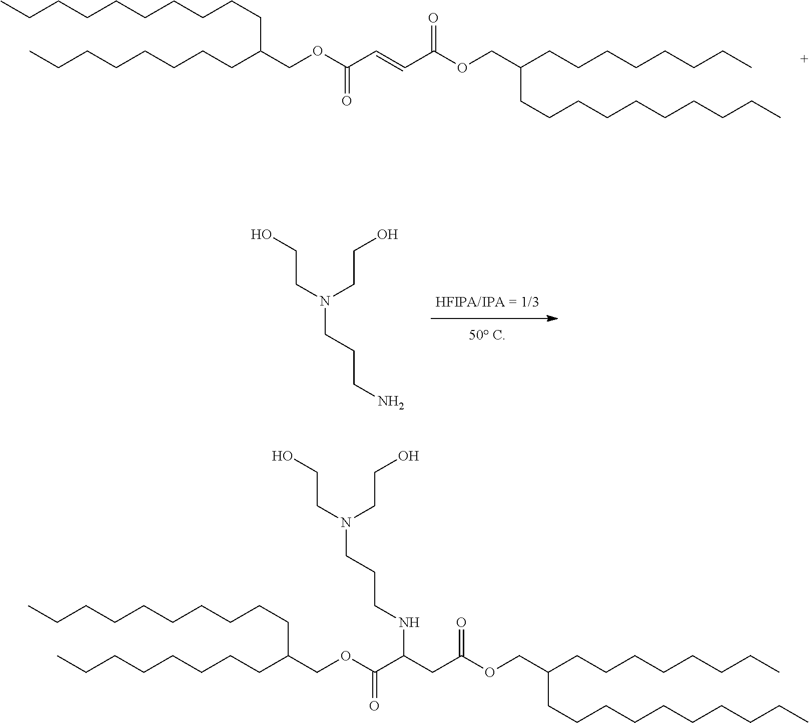

6.0 g 2-octyl-1-dodecanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC when the 2-octyl-1-dodecanol was consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane (85% yield, 5.8 g).

2.0 g di(2-octyldodecyl) fumarate (1 eq.) and 720 mg N-(3-aminopropyl)diethanolamine (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 0% to 33% ethyl acetate in hexane. The product was collected as a pale-yellow oil (40% yield, 990 mg). ¹H NMR (500 MHz, CDCl₃) δ 4.08-3.93 (m, 4H), 3.62 (td, J=5.1, 2.9 Hz, 4H), 3.56 (t, J=6.3 Hz, 1H), 2.79 (ddd, J=10.9, 8.2, 4.4 Hz, 1H), 2.69 (qd, J=16.1, 6.3 Hz, 3H), 2.62 (s, 7H), 1.78-1.69 (m, 1H), 1.61 (dt, J=10.5, 5.0 Hz, 4H), 1.25 (s, 65H), 0.87 (t, J=6.9 Hz, 12H).

### Example 45

### Synthesis of Compound 359

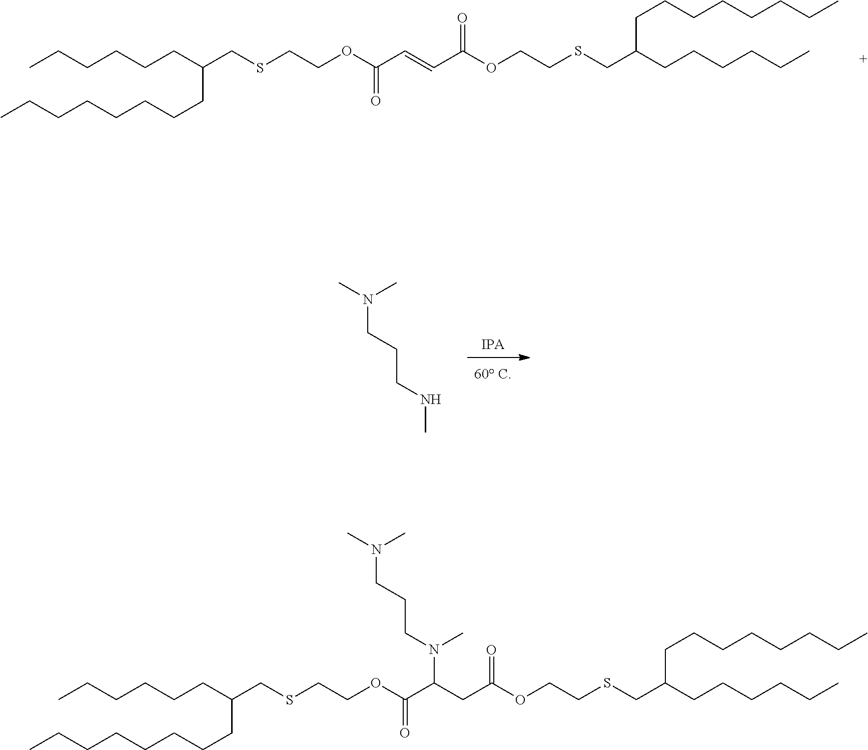

2.6 g 2-mercaptoethanol (1.1 eq) was mixed with 6.5 g Cs₂CO₃ (2.0 eq) and 30 mL methanol under room temperature. After this mixture was stirred for 30 min, 7.3 g 1-bromo-2-hexyldecane (1 eq) was added, and the mixture was stirred overnight under room temperature. The reaction endpoint was confirmed by TLC. Then the reaction mixture was concentrated until most of the methanol was removed, and the slurry was re-dissolved in 50 mL DI water. This mixture was extracted 3 times with 40 mL ethyl acetate. All organic layers were combined, dried over anhydrous Na₂SO₄, and then the solvent was removed. The brown-red viscous crude residue was taken on without further purification. 6.0 g crude 2-((2-hexyl-1-decyl)thio)ethanol (2 eq.) and 3.9 g diisopropyl ethyl amine (3 eq) were mixed with 20 mL anhydrous DCM in a 100 mL round bottom flask. The mixture was cooled to −0° C. with ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane (85% yield, 5.8 g).

2.0 g of the purified intermediate (1 eq.) and 1.0 g N,N,N′-Trimethyl-1,3-propanediamine (3 eq.) were mixed with 5 mL of IPA (isopropyl alcohol) in a 25 mL round bottom flask. The mixture was heated to 60° C. with an oil bath for ~20 h. The reaction was monitored by TLC and ended when the starting materials were consumed. Then the solvent was removed by vacuum distillation, to yield the yellow oily residue as the crude product. The crude product was purified by silica column chromatography, with a mobile phase composed of 0% to 33% ethyl acetate in hexane. The product was collected as a pale-yellow oil (20% yield, 470 mg). ¹H NMR (500 MHz, CDCl₃) δ 4.32-4.17 (m, 4H), 3.82 (dd, J=8.2, 6.7 Hz, 1H), 2.84 (dd, J=16.0, 8.2 Hz, 1H), 2.70 (dt, J=10.3, 7.1 Hz, 4H), 2.62-2.47 (m, 7H), 2.30 (s, 3H), 2.27-2.22 (m, 2H), 2.20 (s, 6H), 1.67-1.55 (m, 2H), 1.26 (s, 48H), 0.90-0.85 (m, 12H).

### Example 46

### Synthesis of Compound 373

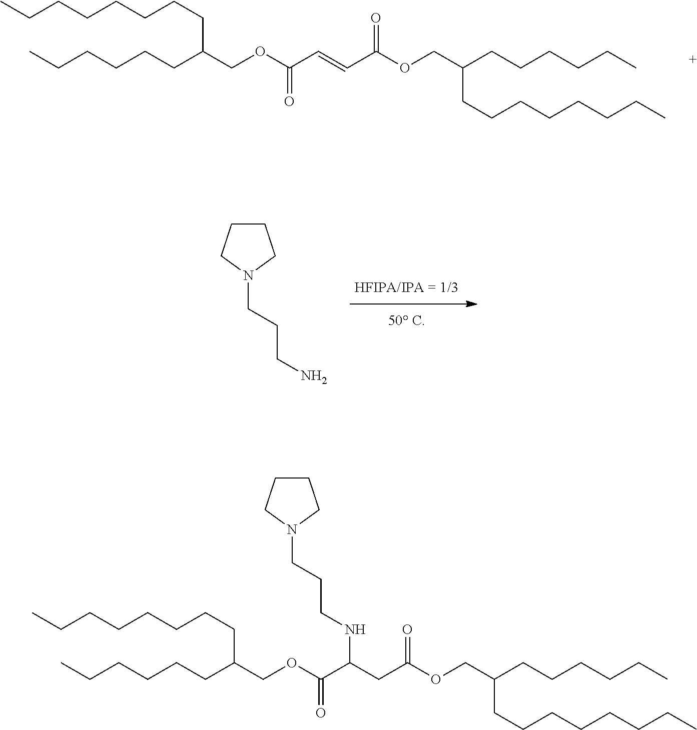

4.8 g 2-hexyl-1-decanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC when the starting materials were consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane to yield the di(2-hexyldecyl) fumarate (85% yield, 4.8 g).

1.7 g di(2-hexyldecyl) fumarate (1 eq.) and 570 mg 1-(3-aminopropyl)pyrrolidine (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 0% to 33% ethyl acetate in hexane as a colorless oil (40% yield, 830 mg). ¹H NMR (500 MHz, CDCl₃) δ 4.08-3.91 (m, 4H), 3.61 (t, J=6.5 Hz, 1H), 2.71 (ddd, J=15.4, 7.5, 5.3 Hz, 2H), 2.66-2.41 (m, 7H), 1.76 (pent, J=3.1 Hz, 4H), 1.71-1.54 (m, 4H), 1.26 (d, J=6.2 Hz, 50H), 0.87 (t, J=6.8 Hz, 12H).

### Example 47

### Synthesis of Compound 374

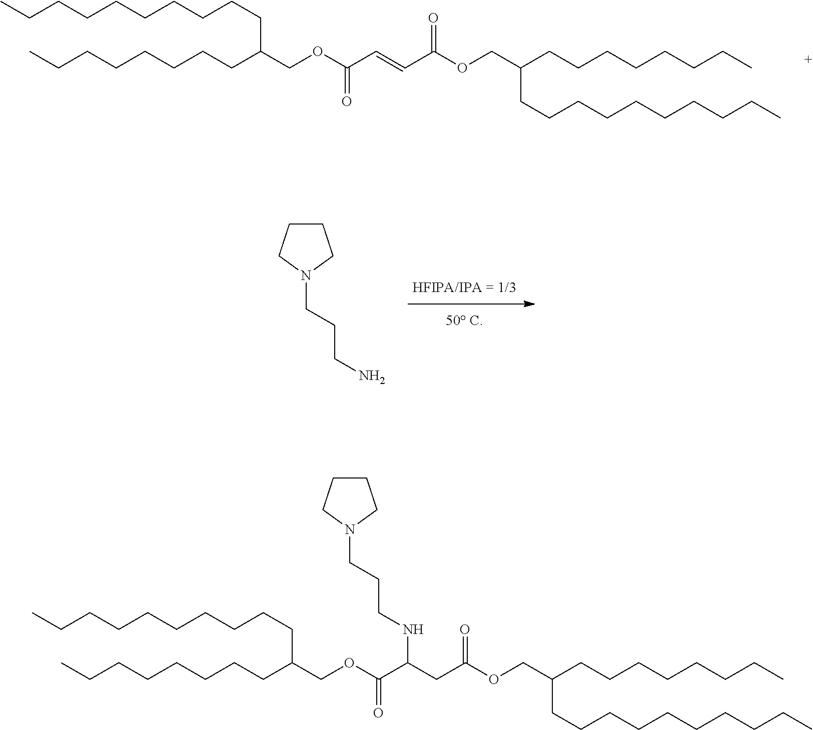

0 g 2-octyl-1-dodecanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC when the 2-octyl-1-dodecanol was consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane (85% yield, 5.8 g).

2.0 g di(2-octyldodecyl) fumarate (1 eq.) and 570 mg 1-(3-aminopropyl)pyrrolidine (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield the yellow oily residue as the crude product. The crude product was purified by silica column chromatography, with a mobile phase composed of 0% to 33% ethyl acetate in hexane. The product was a colorless oil (40%, 950 mg). ¹H NMR (500 MHz, CDCl₃) δ=4.00 (dt, J=25.0, 4.0 Hz, 4H), 3.61 (d, J=6.5 HZ, 1H), 2.71 (dt, J=16.3, 4.7 Hz, 2H), 2.62 (dt, 14.8, 5.0 Hz, 1H), 2.53 (dd, J=105., 7.0 Hz, 1H), 2.46 (h, J=3.3 Hz, 6H), 1.75 (pent, J=3.2 Hz, 4H), 1.72-15.3 (m, 4H), 1.25 (s, 65H), 0.87 (tt, J=5.8, 2.6 Hz).

### Example 48

### Synthesis of Compound 375

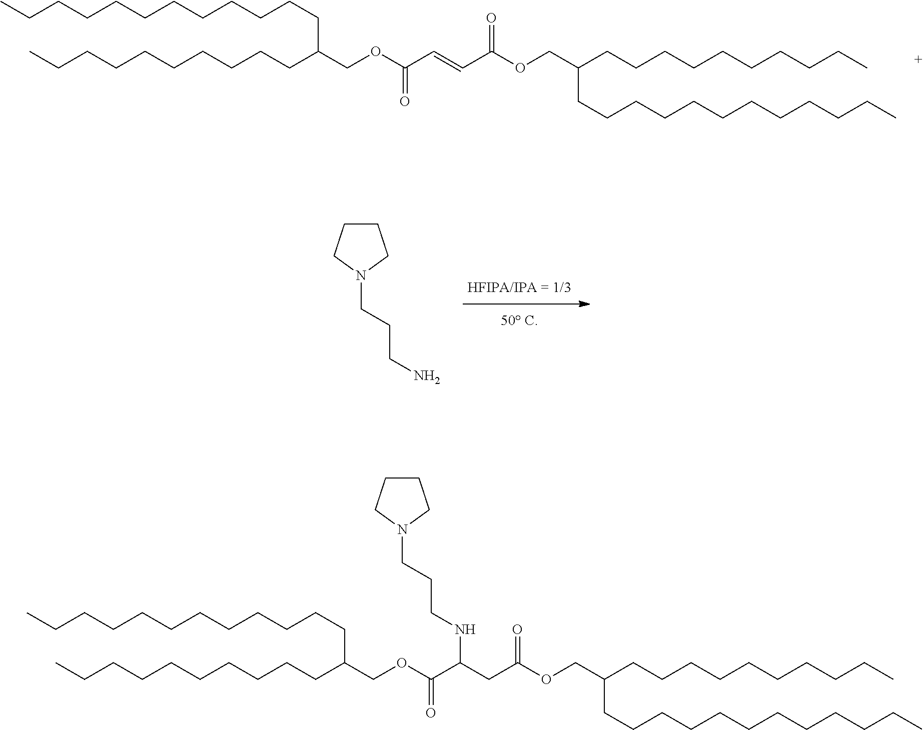

7.1 g 2-decyl-1-tetradecanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane (85%, 6.7 g).

2.3 g di(2-decyltetradecyl) fumarate (1 eq.) and 570 mg 1-(3-aminopropyl)pyrrolidine (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 0% to 33% ethyl acetate in hexane. The product was collected as a pale-yellow oil (40% yield, 1070 mg). ¹H NMR (500 MHz, CDCl₃) δ 4.07-3.92 (m, 4H), 3.61 (t, J=6.5 Hz, 1H), 2.77-2.66 (m, 2H), 2.66-2.51 (m, 2H), 2.48 (d, J=6.6 Hz, 5H), 1.76 (pent, J=3.0 Hz, 4H), 1.72-1.54 (m, 4H), 1.25 (s, 82H), 0.87 (t, J=6.7 Hz, 12H).

### Example 49

### Synthesis of Compound 380

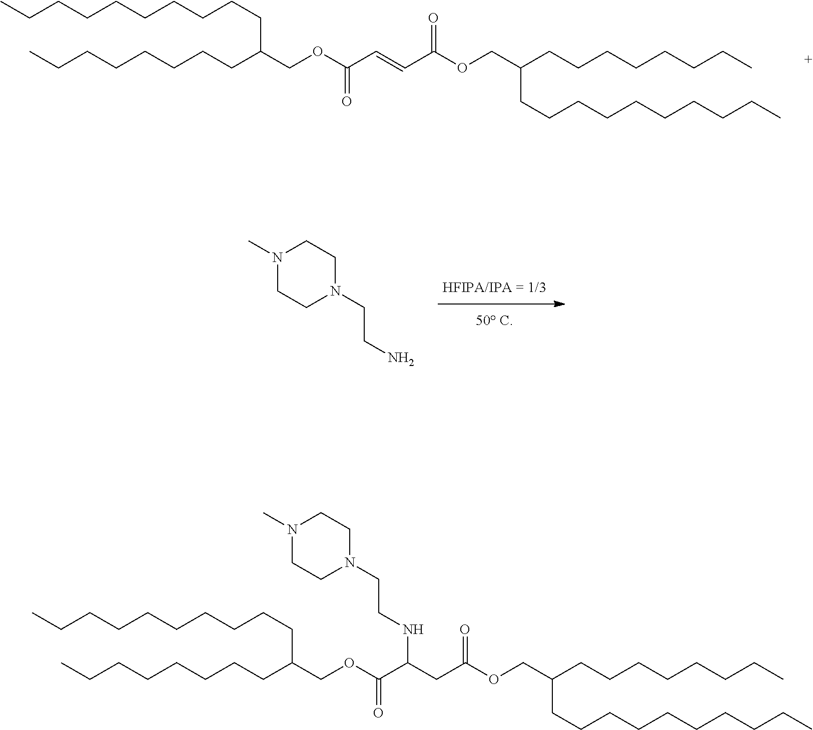

0.0 g 2-octyl-1-dodecanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC when the 2-octyl-1-dodecanol was consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane (85% yield, 5.8 g). 2.0 g di(2-octyldodecyl) fumarate (1 eq.) and 635 mg 2-(4-Methyl-piperazin-1-yl)-ethylamine (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield the yellow oily residue as the crude product. The crude product was purified by silica column chromatography, with a mobile phase composed of 0% to 33% ethyl acetate in hexane as a pale-yellow oil (30% yield, 730 mg). ¹H NMR (500 MHz, CDCl₃) δ 4.07-3.91 (m, 4H), 3.65 (t, J=6.6 Hz, 1H), 2.83-2.67 (m, 2H), 2.67-2.54 (m, 3H), 2.54-2.31 (m, 8H), 2.26 (d, J=1.1 Hz, 3H), 1.97 (s, 2H), 1.61 (s, 2H), 1.35-1.06 (m, 64H), 0.87 (t, J=6.8 Hz, 12H).

### Example 50

### Synthesis of Compound 441

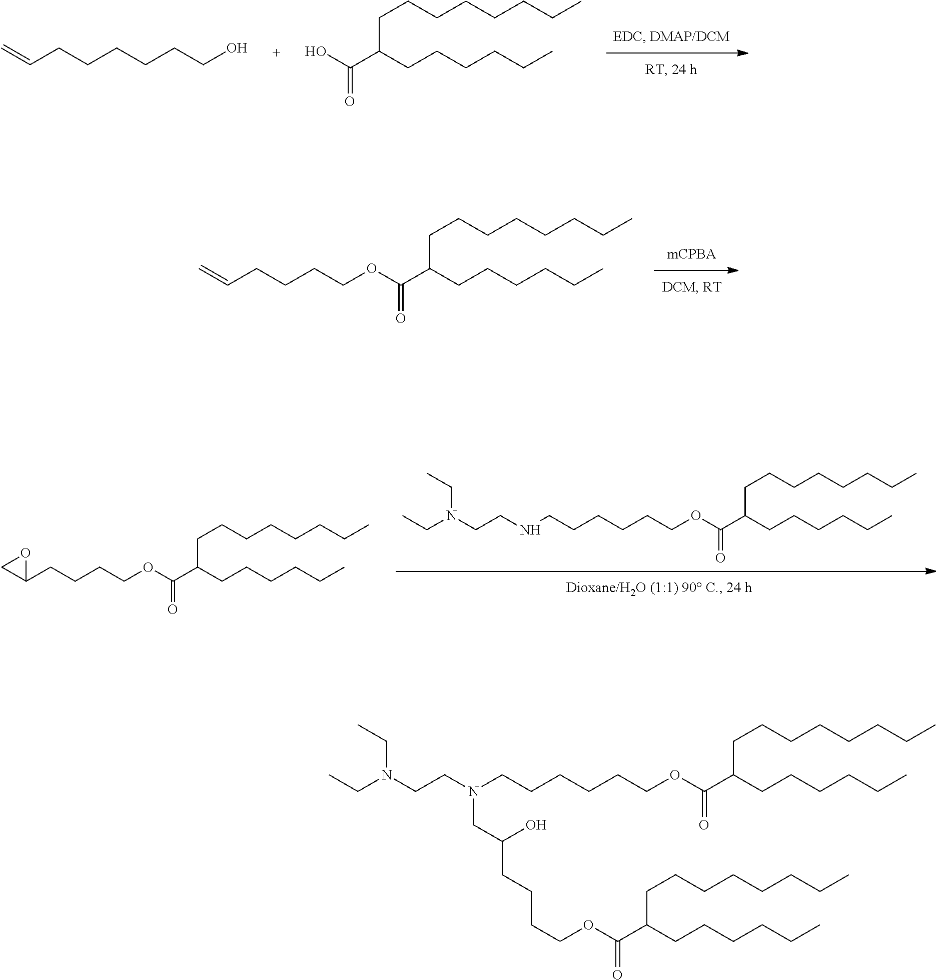

2-Hexyldecanoic acid (5.12 g, 20 mmol), EDC-HCl (4.97 g, 26 mmol) and DMAP (3.17 g, 26 mmol) was stirred in anhydrous DCM for 20 minutes at room temperature. 5-Hexen-1-ol (2.0 g, 20 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (20 mL×2). The hexane layer was separated, dried over anhydrous Na₂SO₄, and evaporated under vacuum to afford hex-5-en-1-yl 2-hexyldecanoate as colorless oil (4.21 g, 62%). The product was used without further purification.

To a solution of ester (10 mmol) in anhydrous DCM was added mCPBA (2.51 g, 11 mmol). The resulting mixture was stirred at room temperature overnight. DCM was removed under vacuum. 100 mL of hexane was added to the residue, and the mixture was washed with saturated Na₂S₂O₃, saturated Na₂CO₃, brine, dried over anhydrous Na₂SO₄. The solvent was removed under vacuum to afford 4-(oxiran-2-yl)butyl 2-hexyldecanoate as colorless oil (2.12 g, 59%). The crude product was used without further purification. 4-(oxiran-2-yl)butyl 2-hexyldecanoate (71 mg, 0.2 mmol) and 2-hexyldecyl 6-(diethylaminoethyl)aminohexanoate (91 g, 0.2 mmol) in a mixture of 1,4-dioxane and H₂O (v:v, 1:1, 1 mL) was heated to 90° C. and stirred for 24 hours. The solvents were removed under vacuum and the residue was purified by silica-gel chromatography (5% MeOH in DCM with 0.1% ammonia hydroxide) to afford the product as light brown oil (198 mg, 48%). ¹H NMR (500 MHz, CDCl₃) δ 4.16-3.99 (m, 3H), 3.95 (d, J=5.7 Hz, 2H), 3.53 (d, J=6.1 Hz, 1H), 2.78-2.36 (m, 9H), 2.34-2.22 (m, 4H), 2.03 (d, J=0.8 Hz, 1H), 1.70-1.49 (m, 8H), 1.41 (dt, J=14.1, 8.7 Hz, 7H), 1.25 (qd, J=7.2, 4.7 Hz, 47H), 1.04 (t, J=7.1 Hz, 6H), 0.86 (qd, J=5.9, 2.6 Hz, 12H).

### Example 51

### Synthesis of Compound 442

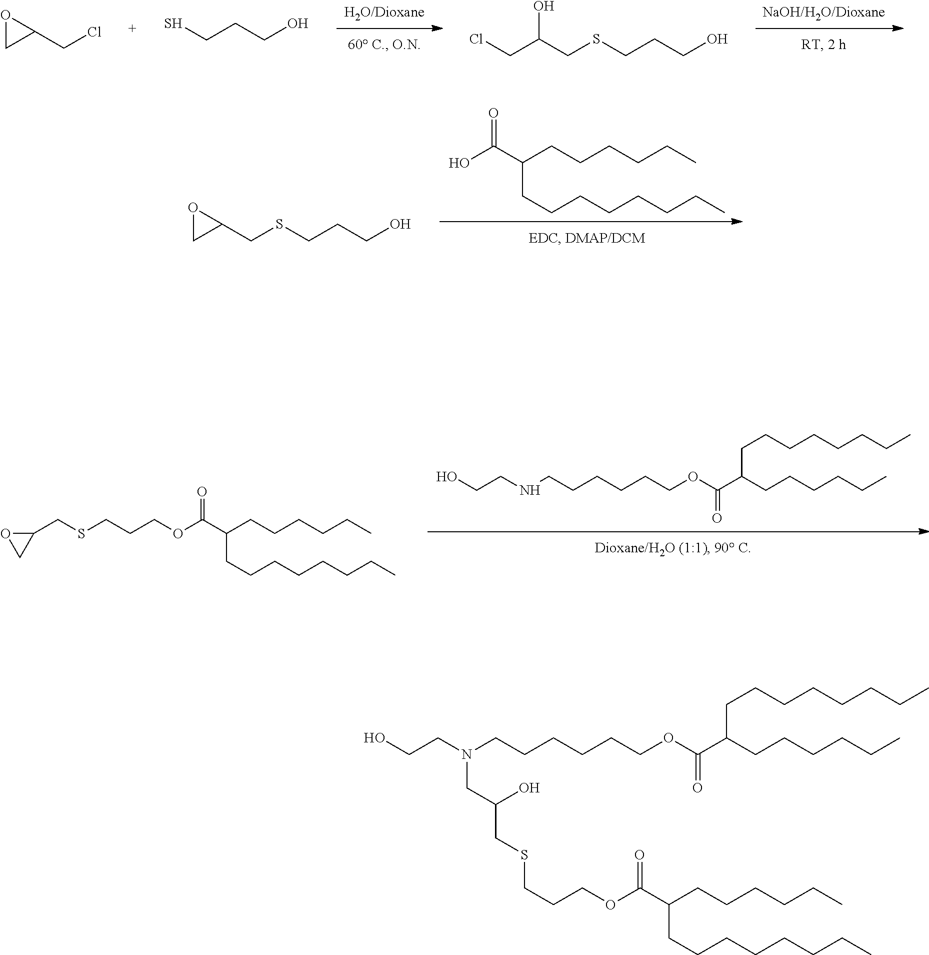

(±)-epichlorohydrin (2.04, 22 mmol) and 3-mercaptopropanol (1.84 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature. 100 mL H₂O was added, the mixture was extracted with DCM (50 mL×3). DCM layers were combined, dried over anhydrous Na₂SO₄, and DCM was removed under vacuum to afford 3-((oxiran-2-ylmethyl)thio)propan-1-ol as colorless oil (1.82 g, 70%). The crude product was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 3-((oxiran-2-ylmethyl)thio)propan-1-ol (1.48 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford 3-((oxiran-2-ylmethyl)thio)propyl 2-hexyldecanoate as colorless oil (2.12 g, 54%). The crude product was used without further purification. 3-((oxiran-2-ylmethyl)thio)propyl 2-hexyldecanoate (190 mg, 0.5 mmol) and 2-hexyldecyl 6-((2-hydroxyethyl)amino)hexanoate (198 mg, 0.5 mmol) in a mixture of 1,4-dioxane and H₂O (v:v, 1:1, 1 mL) was heated to 90° C. and stirred for 24 hours. The solvents were removed under vacuum and the residue was purified by silica-gel chromatography (5% MeOH in DCM with 0.1% ammonia hydroxide) to afford the product as light brown oil (175 mg. 44%). ¹H NMR (500 MHz, CDCl₃) δ 4.14 (dd, J=16.0, 9.6 Hz, 2H), 3.95 (d, J=5.7 Hz, 2H), 3.77 (s, 2H), 3.68-3.47 (m, 3H), 2.77-2.68 (m, 2H), 2.66-2.54 (m, 6H), 2.49 (dd, J=12.8, 8.5 Hz, 2H), 2.36-2.25 (m, 3H), 2.18-2.01 (m, 2H), 1.95-1.85 (m, 2H), 1.69-1.51 (m, 6H), 1.52-1.36 (m, 5H), 1.37-1.17 (m, 43H), 0.87 (td, J=6.6, 2.7 Hz, 12H).

### Example 52

### Synthesis of Compound 443

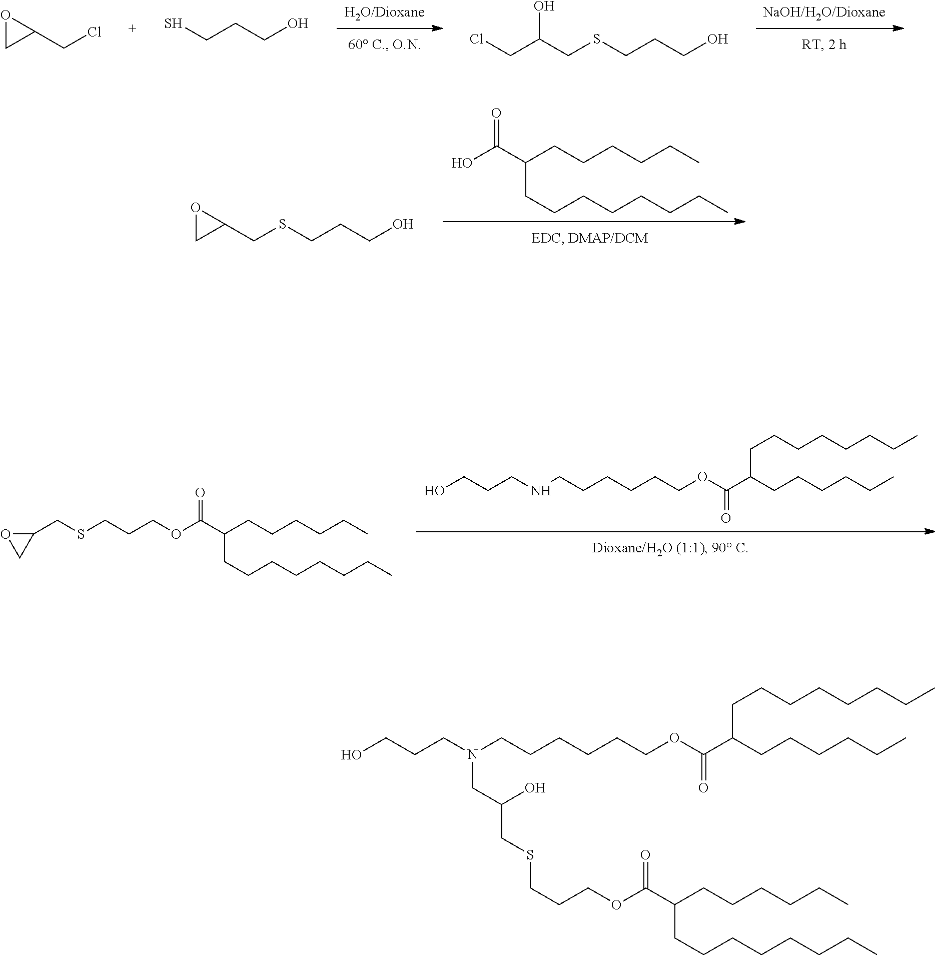

(±)-epichlorohydrin (2.04, 22 mmol) and 3-mercaptopropanol (1.84 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature. 100 mL H₂O was added, the mixture was extracted with DCM (50 mL×3). DCM layers were combined, dried over anhydrous Na₂SO₄, and DCM was removed under vacuum to afford 3-((oxiran-2-ylmethyl)thio)propan-1-ol as colorless oil (1.82 g, 70%). The crude product was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 3-((oxiran-2-ylmethyl)thio)propan-1-ol (1.48 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford 3-((oxiran-2-ylmethyl)thio)propyl 2-hexyldecanoate as colorless oil (2.12 g, 54%). The crude product was used without further purification.

3-((oxiran-2-ylmethyl)thio)propyl 2-hexyldecanoate (190 mg, 0.5 mmol) and 2-hexyldecyl 6-((3-hydroxypropyl)amino)hexanoate (201 mg, 0.5 mmol) in a mixture of 1,4-dioxane and H₂O (v:v, 1:1, 1 mL) was heated to 90° C. and stirred for 24 hours. The solvents were removed under vacuum and the residue was purified by silica-gel chromatography (5% MeOH in DCM with 0.1% ammonia hydroxide) to afford the product as light brown oil (174 mg, 43%). ¹H NMR (500 MHz, CDCl₃) δ 4.15 (t, J=6.2 Hz, 3H), 4.08 (s, 1H), 3.95 (d, J=5.8 Hz, 2H), 3.81 (s, 1H), 3.77-3.67 (m, 2H), 2.81-2.68 (m, 2H), 2.68-2.35 (m, 9H), 2.29 (t, J=7.2 Hz, 3H), 2.07 (d, J=36.3 Hz, 1H), 1.97-1.87 (m, 2H), 1.80-1.52 (m, 8H), 1.49 (s, 1H), 1.42 (s, 3H), 1.28 (d, J=37.7 Hz, 43H), 0.92-0.79 (m, 12H).

### Example 53

### Synthesis of Compound 444

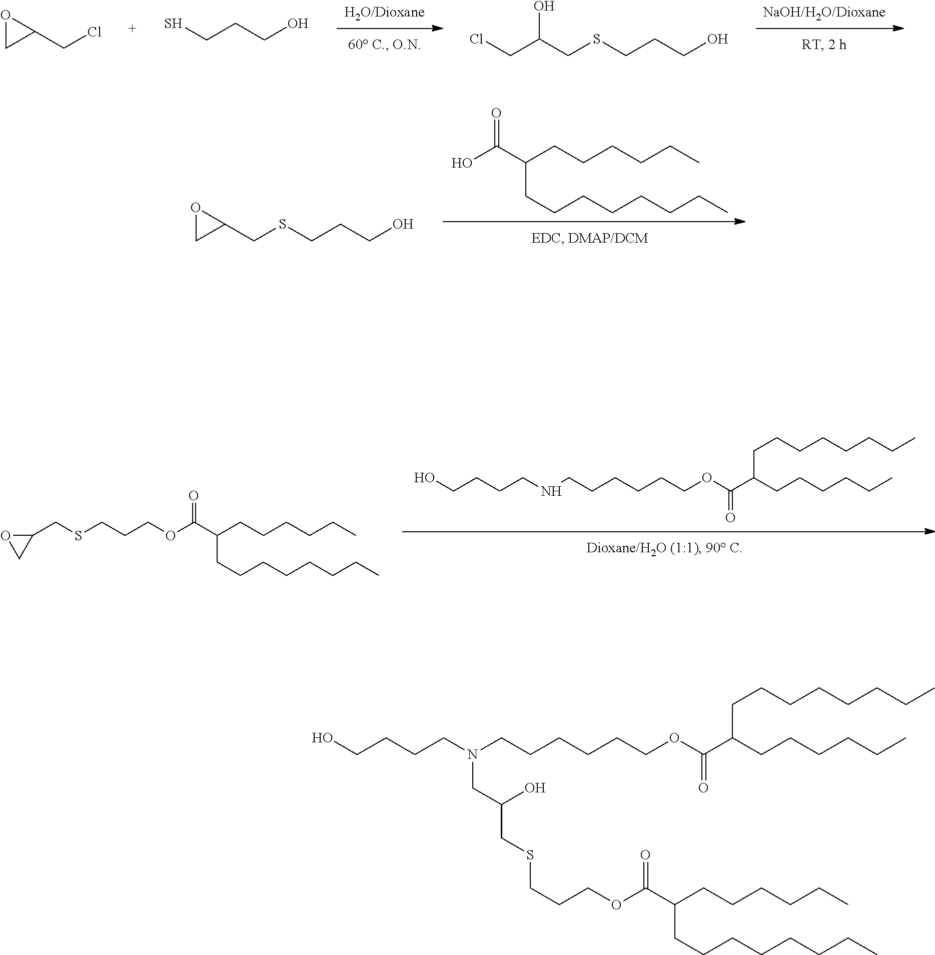

(±)-epichlorohydrin (2.04, 22 mmol) and 3-mercaptopropanol (1.84 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature. 100 mL H₂O was added, the mixture was extracted with DCM (50 mL×3). DCM layers were combined, dried over anhydrous Na₂SO₄, and DCM was removed under vacuum to afford 3-((oxiran-2-ylmethyl)thio)propan-1-ol as colorless oil (1.82 g, 70%). The crude product was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 3-((oxiran-2-ylmethyl)thio)propan-1-ol (1.48 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford 3-((oxiran-2-ylmethyl)thio)propyl 2-hexyldecanoate as colorless oil (2.12 g, 54%). The crude product was used without further purification.

3-((oxiran-2-ylmethyl)thio)propyl 2-hexyldecanoate (190 mg, 0.5 mmol) and 2-hexyldecyl 6-((4-hydroxybutyl)amino)hexanoate (210 mg, 0.5 mmol) in a mixture of 1,4-dioxane and H₂O (v:v, 1:1, 1 mL) was heated to 90° C. and stirred for 24 hours. The solvents were removed under vacuum and the residue was purified by silica-gel chromatography (5% MeOH in DCM with 0.1% ammonia hydroxide) to afford the product as light brown oil (192 mg, 47%). ¹H NMR (500 MHz, CDCl₃) δ 4.15 (t, J=6.3 Hz, 2H), 3.95 (d, J=5.8 Hz, 2H), 3.80 (s, 2H), 3.60 (d, J=14.4 Hz, 3H), 2.68-2.60 (m, 2H), 2.60-2.50 (m, 4H), 2.46 (dd, J=20.8, 8.0 Hz, 4H), 2.29 (t, J=7.3 Hz, 4H), 1.97-1.87 (m, 2H), 1.69-1.52 (m, 9H), 1.50 (s, 2H), 1.42 (s, 3H), 1.25 (d, J=5.2 Hz, 44H), 0.86 (dd, J=7.0, 3.2 Hz, 12H).

### Example 54

### Synthesis of Compound 445

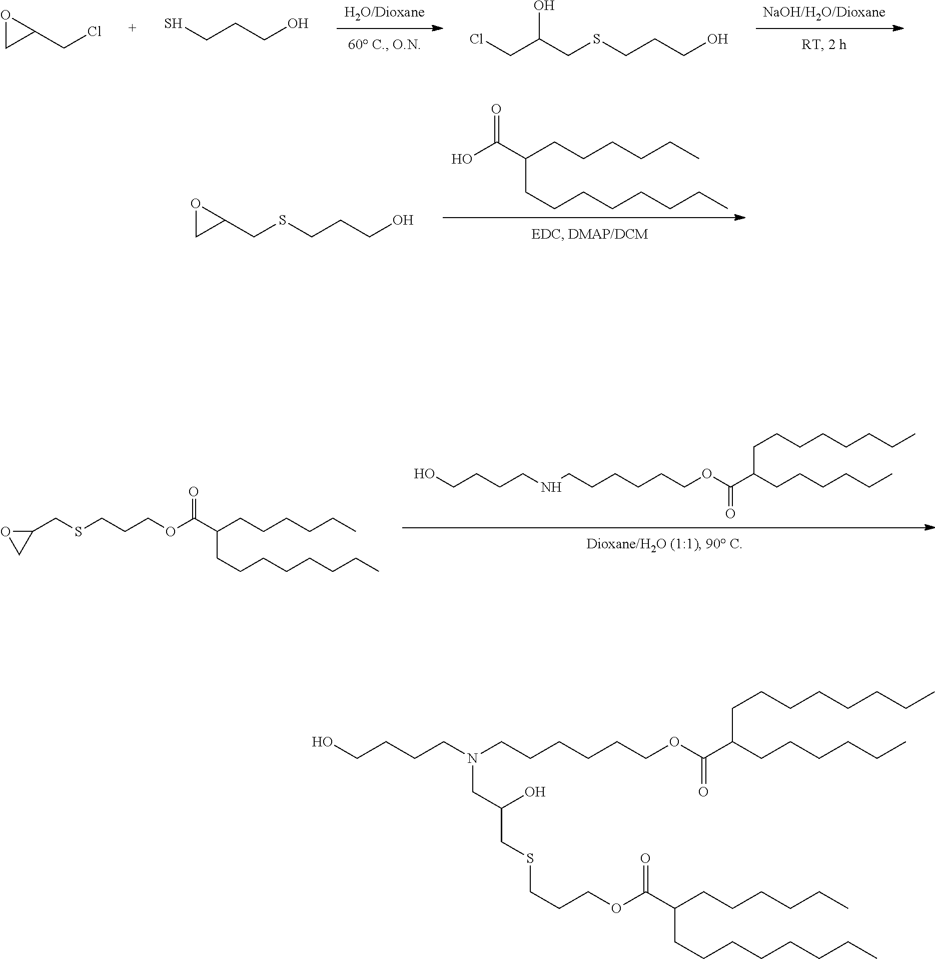

(±)-epichlorohydrin (2.04, 22 mmol) and 3-mercaptopropanol (1.84 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature. 100 mL H₂O was added, the mixture was extracted with DCM (50 mL×3). DCM layers were combined, dried over anhydrous Na₂SO₄, and DCM was removed under vacuum to afford 3-((oxiran-2-ylmethyl)thio)propan-1-ol as colorless oil (1.82 g, 70%). The crude product was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 3-((oxiran-2-ylmethyl)thio)propan-1-ol (1.48 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford 3-((oxiran-2-ylmethyl)thio)propyl 2-hexyldecanoate as colorless oil (2.12 g, 54%). The crude product was used without further purification.

3-((oxiran-2-ylmethyl)thio)propyl 2-hexyldecanoate (184 mg, 0.5 mmol) and 6-((4-hydroxybutyl)amino)hexyl 2-hexyldecanoate (210 mg, 0.5 mmol) in a mixture of 1,4-dioxane and H₂O (v:v, 1:1, 1 mL) was heated to 90° C. and stirred for 24 hours. The solvents were removed under vacuum and the residue was purified by silica-gel chromatography (5% MeOH in DCM with 0.1% ammonia hydroxide) to afford the product as light brown oil (172 mg, 42%). ¹H NMR (500 MHz, CDCl₃) δ 4.15 (t, J=6.3 Hz, 2H), 4.04 (t, J=6.7 Hz, 2H), 3.80 (s, 2H), 3.64-3.55 (m, 2H), 2.68-2.60 (m, 2H), 2.60-2.50 (m, 4H), 2.50-2.36 (m, 4H), 2.29 (dd, J=8.6, 5.5 Hz, 3H), 1.91 (dt, J=13.5, 6.6 Hz, 3H), 1.69-1.51 (m, 10H), 1.41 (ddd, J=22.8, 20.6, 7.7 Hz, 9H), 1.24 (s, 40H), 0.86 (t, J=6.8 Hz, 12H).

### Example 55

### Synthesis of Compound 446

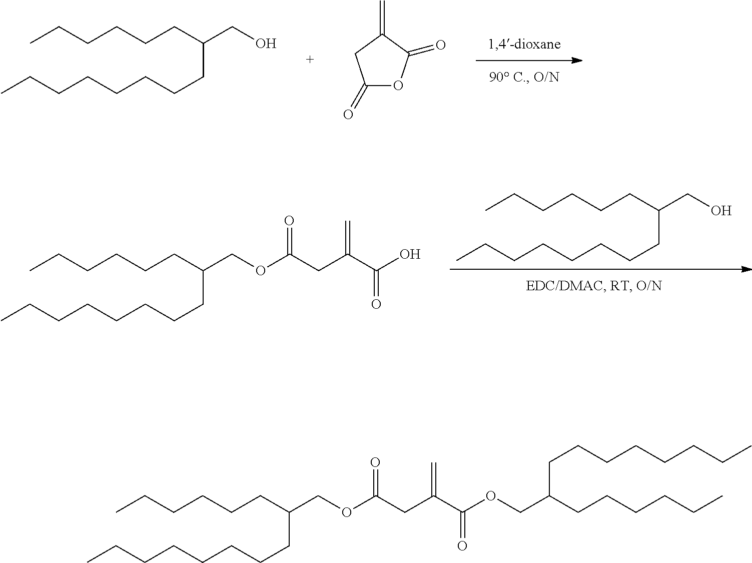

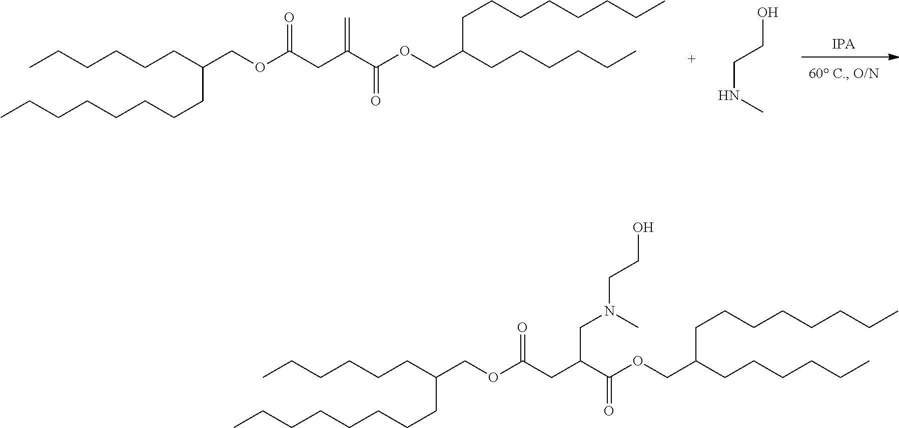

4.8 g 2-hexyl-1-decanol (1 eq.) and 2.35 itaconic anhydride (1.05 eq) were mixed with 10 mL dioxane in a 100 mL round bottom flask. After fully dissolved, this mixture was heated to 90° C. and stirred overnight. The reaction endpoint was confirmed by TLC when the starting materials were consumed. Then solvent was removed by vacuum distillation, and the residue was β-mono(2-hexyldecyl)itaconate (99% yield, 7.1 g). The product was used without further purification.

5.0 g 2-hexyl-1-decanol (1.05 eq), 4.6 g 1-ethyl-3-(3-dimethylaminopropyl)carbodiimide hydrochloride (EDC, 1.2 eq) and 2.9 g 4-dimethylaminopyridine (DMAP, 1.2 eq) were mixed with β-mono(2-hexyldecyl)itaconate (7.1 g, 1 eq) and 30 mL of DCM in a 100 mL round bottom flask. The mixture immediately turned to deep red and was stirred overnight at room temperature. The reaction endpoint was confirmed by TLC when the starting materials were consumed. The reaction mixture was diluted with 70 mL ethyl acetate and washed 3 times with DI water and saturated NaCl solution, subsequently. Then the organic layer was separated and dried by vacuum distillation. The residue was purified by silica column chromatography with hexane to yield the bis(2-hexyldecyl)itaconate (70% yield, 8.1 g).

2.0 g bis(2-hexyldecyl)itaconate (1 eq.) and 780 mg N-methylethanolamine (3.0 eq) were mixed with 5 mL of IPA (isopropyl alcohol) in a 25 mL round bottom flask. The mixture was heated to 60° C. with an oil bath for overnight. The reaction was monitored by TLC and ended when the starting materials were consumed. Then the solvent was removed by vacuum distillation, to yield the deep red oily residue as the crude product. The crude product was purified by silica column chromatography, with a mobile phase composed of 0% to 20% ethyl acetate in hexane. The product was collected as a red oil (30%, 680 mg). ¹H NMR (500 MHz, CDCl₃) δ 4.06-3.88 (m, 4H), 3.56 (q, J=5.7 Hz, 2H), 3.08 (pent, J=7.1 Hz, 1H), 2.77-2.59 (m, 3H), 2.59-2.43 (m, 4H), 2.26 (d, J=1.1 Hz, 3H), 1.61 (s, 2H), 1.26 (d, J=6.7 Hz, 48H), 0.87 (t, J=6.7 Hz, 12H).

### Example 56

### Synthesis of Compound 447

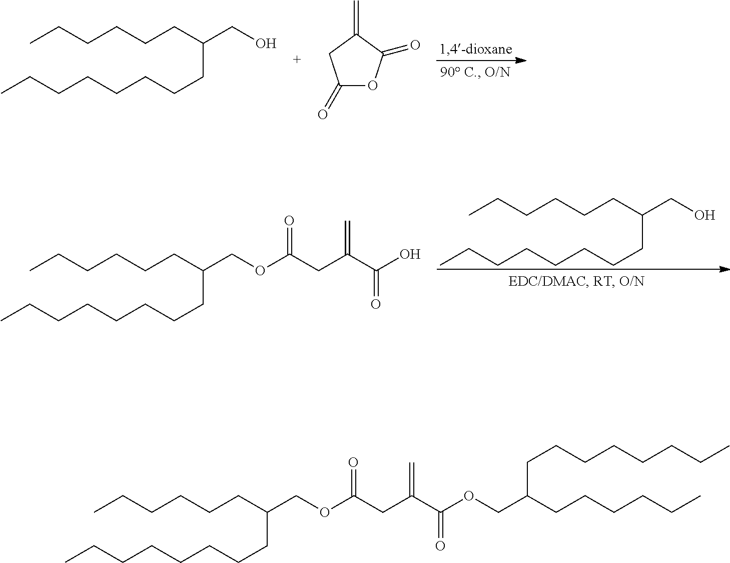

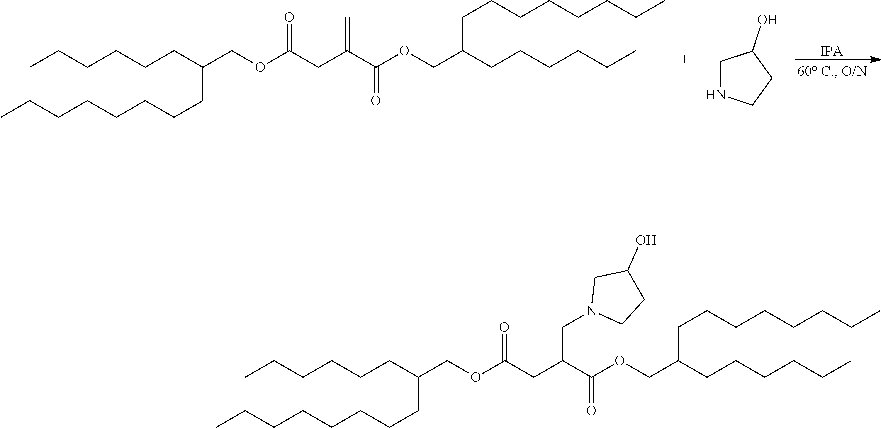

4.8 g 2-hexyl-1-decanol (1 eq.) and 2.35 itaconic anhydride (1.05 eq) were mixed with 10 mL dioxane in a 100 mL round bottom flask. After fully dissolved, this mixture was heated to 90° C. and stirred overnight. The reaction endpoint was confirmed by TLC when the starting materials were consumed. Then solvent was removed by vacuum distillation, and the residue was β-mono(2-hexyldecyl)itaconate (99% yield, 7.1 g). The product was used without further purification.

5.0 g 2-hexyl-1-decanol (1.05 eq), 4.6 g 1-ethyl-3-(3-dimethylaminopropyl)carbodiimide hydrochloride (EDC, 1.2 eq) and 2.9 g 4-dimethylaminopyridine (DMAP, 1.2 eq) were mixed with β-mono(2-hexyldecyl)itaconate (7.1 g, 1 eq) and 30 mL of DCM in a 100 mL round bottom flask. The mixture immediately turned to deep red and was stirred overnight at room temperature. The reaction endpoint was confirmed by TLC when the starting materials were consumed. The reaction mixture was diluted with 70 mL ethyl acetate and washed 3 times with DI water and saturated NaCl solution, subsequently. Then the organic layer was separated and dried by vacuum distillation. The residue was purified by silica column chromatography with hexane to yield the bis(2-hexyldecyl)itaconate (70% yield, 8.1 g).

2.0 g bis(2-hexyldecyl)itaconate (1 eq.) and 900 mg 3-hydroxy pyrrolidine (3.0 eq) were mixed with 5 mL of IPA (isopropyl alcohol) in a 25 mL round bottom flask. The mixture was heated to 60° C. with an oil bath for overnight. The reaction was monitored by TLC and ended when the starting materials were consumed. Then the solvent was removed by vacuum distillation, to yield the deep red oily residue as the crude product. The crude product was purified by silica column chromatography, with a mobile phase composed of 0% to 20% ethyl acetate in hexane. The product was collected as a red oil (20%, 460 mg). ¹H NMR (500 MHz, CDCl₃) δ 4.27 (s, 1H), 4.07-3.88 (m, 4H), 3.08-2.98 (m, 1H), 2.93-2.83 (m, 1H), 2.79-2.58 (m, 4H), 2.58-2.45 (m, 1H), 2.30 (q, J=7.8 Hz, 1H), 2.19-2.06 (m, 1H), 1.90 (s, 1H), 1.77-1.66 (m, 1H), 1.61 (s, 2H), 1.27 (d, J=8.1 Hz, 48H), 0.87 (t, J=6.8 Hz, 13H).

### Example 57

### Synthesis of Compound 448

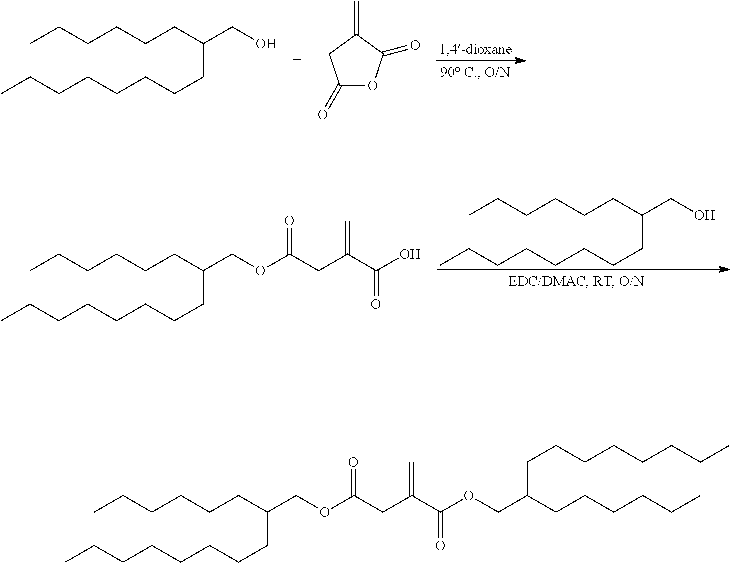

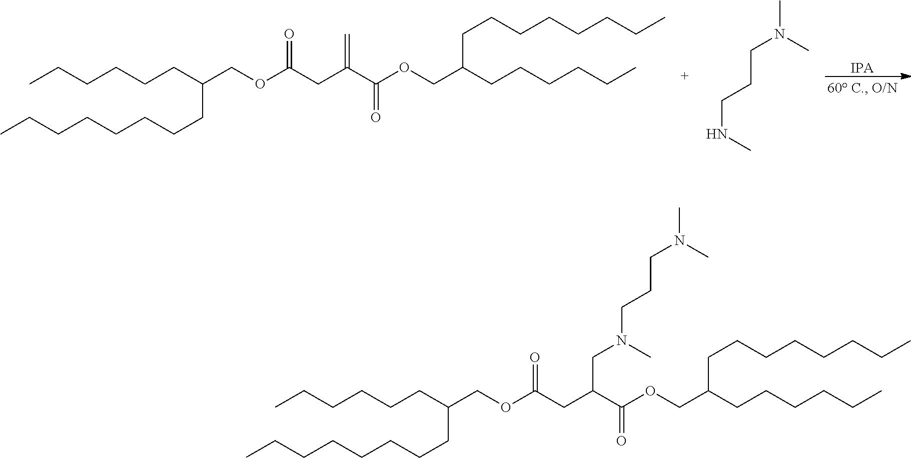

4.8 g 2-hexyl-1-decanol (1 eq.) and 2.35 itaconic anhydride (1.05 eq) were mixed with 10 mL dioxane in a 100 mL round bottom flask. After fully dissolved, this mixture was heated to 90° C. and stirred overnight. The reaction endpoint was confirmed by TLC when the starting materials were consumed. Then solvent was removed by vacuum distillation, and the residue was β-mono(2-hexyldecyl)itaconate (99% yield, 7.1 g). The product was used without further purification.

5.0 g 2-hexyl-1-decanol (1.05 eq), 4.6 g 1-ethyl-3-(3-dimethylaminopropyl)carbodiimide hydrochloride (EDC, 1.2 eq) and 2.9 g 4-dimethylaminopyridine (DMAP, 1.2 eq) were mixed with β-mono(2-hexyldecyl)itaconate (7.1 g, 1 eq) and 30 mL of DCM in a 100 mL round bottom flask. The mixture immediately turned to deep red and was stirred overnight at room temperature. The reaction endpoint was confirmed by TLC when the starting materials were consumed. The reaction mixture was diluted with 70 mL ethyl acetate and washed 3 times with DI water and saturated NaCl solution, subsequently. Then the organic layer was separated and dried by vacuum distillation. The residue was purified by silica column chromatography with hexane to yield the bis(2-hexyldecyl)itaconate (70% yield, 8.1 g).

2.0 g bis(2-hexyldecyl)itaconate (1 eq.) and 1.20 g N,N,N′-trimethyl-1,3-propanediamine (3.0 eq) were mixed with 5 mL of IPA (isopropyl alcohol) in a 25 mL round bottom flask. The mixture was heated to 60° C. with an oil bath overnight. The reaction was monitored by TLC and ended when the starting materials were consumed. Then the solvent was removed by vacuum distillation, to yield the deep red oily residue as the crude product. The crude product was purified by silica column chromatography, with a mobile phase composed of 0% to 33% ethyl acetate in hexane. The product was collected as a red oil (40%, 960 mg). ¹H NMR (500 MHz, CDCl₃) δ 4.05-3.88 (m, 4H), 3.07-2.95 (m, 1H), 2.70-2.53 (m, 3H), 2.43-2.29 (m, 3H), 2.25 (dd, J=8.4, 6.8 Hz, 2H), 2.19 (d, J=9.2 Hz, 8H), 1.66-1.50 (m, 4H), 1.26 (d, J=8.8 Hz, 49H), 0.87 (t, J=6.8 Hz, 12H).

### Example 58

### Synthesis of Compound 449

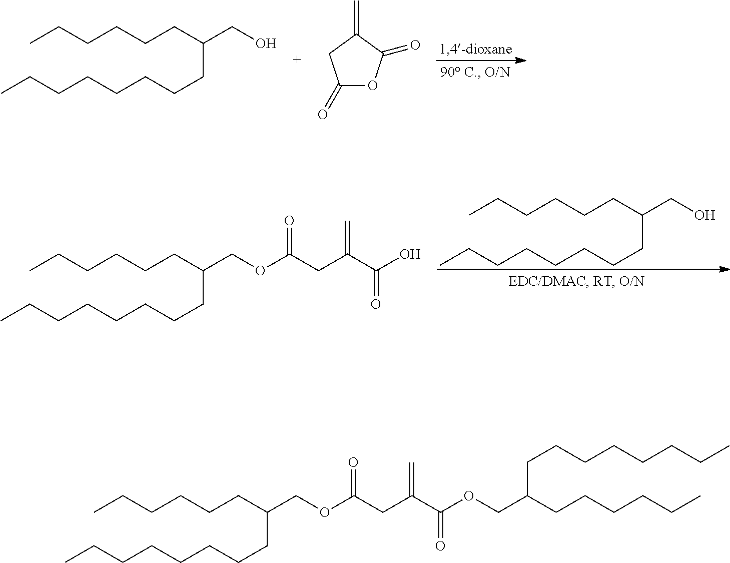

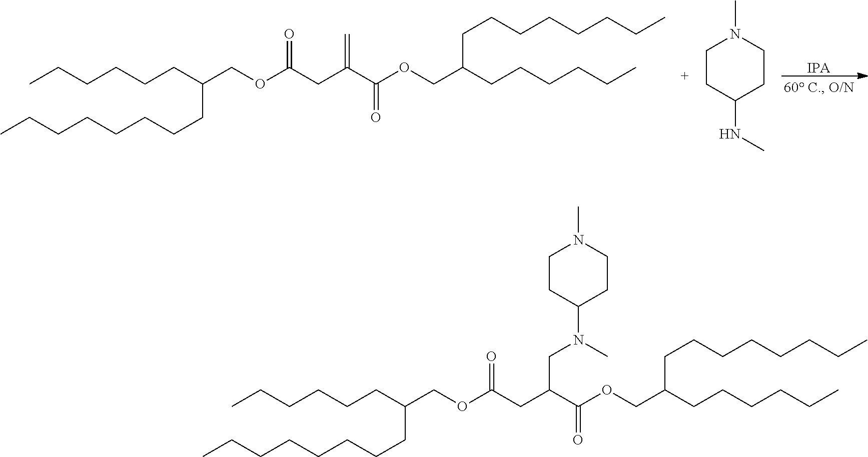

4.8 g 2-hexyl-1-decanol (1 eq.) and 2.35 itaconic anhydride (1.05 eq) were mixed with 10 mL dioxane in a 100 mL round bottom flask. After fully dissolved, this mixture was heated to 90° C. and stirred overnight. The reaction endpoint was confirmed by TLC when the starting materials were consumed. Then solvent was removed by vacuum distillation, and the residue was β-mono(2-hexyldecyl)itaconate (99% yield, 7.1 g). The product was used without further purification.

5.0 g 2-hexyl-1-decanol (1.05 eq), 4.6 g 1-ethyl-3-(3-dimethylaminopropyl)carbodiimide hydrochloride (EDC, 1.2 eq) and 2.9 g 4-dimethylaminopyridine (DMAP, 1.2 eq) were mixed with β-mono(2-hexyldecyl)itaconate (7.1 g, 1 eq) and 30 mL of DCM in a 100 mL round bottom flask. The mixture immediately turned to deep red and was stirred overnight at room temperature. The reaction endpoint was confirmed by TLC when the starting materials were consumed. The reaction mixture was diluted with 70 mL ethyl acetate and washed 3 times with DI water and saturated NaCl solution, subsequently. Then the organic layer was separated and dried by vacuum distillation. The residue was purified by silica column chromatography with hexane to yield the bis(2-hexyldecyl)itaconate (70% yield, 8.1 g).

2.0 g bis(2-hexyldecyl)itaconate (1 eq.) and 1.32 g N,N,N′-trimethyl-1,3-propanediamine (3.0 eq) were mixed with 5 mL of IPA (isopropyl alcohol) in a 25 mL round bottom flask. The mixture was heated to 60° C. with an oil bath overnight. The reaction was monitored by TLC and ended when the starting materials were consumed. Then the solvent was removed by vacuum distillation, to yield the deep red oily residue as the crude product. The crude product was purified by silica column chromatography, with a mobile phase composed of 0% to 33% ethyl acetate in hexane. The product was collected as a red oil (30%, 730 mg). ¹H NMR (500 MHz, CDCl₃) δ 4.07-3.87 (m, 4H), 3.00 (pent, J=7.2 Hz, 1H), 2.86 (d, J=11.5 Hz, 2H), 2.70 (dd, J=12.6, 6.8 Hz, 1H), 2.60 (qd, J=16.6, 6.9 Hz, 2H), 2.49 (dd, J=12.5, 8.3 Hz, 1H), 2.34-2.25 (m, 1H), 2.23 (d, J=8.3 Hz, 5H), 1.97-1.83 (m, 2H), 1.70-1.47 (m, 6H), 1.26 (d, J=5.0 Hz, 49H), 0.87 (t, J=6.8 Hz, 12H).

### Example 59

### Synthesis of Compound 450

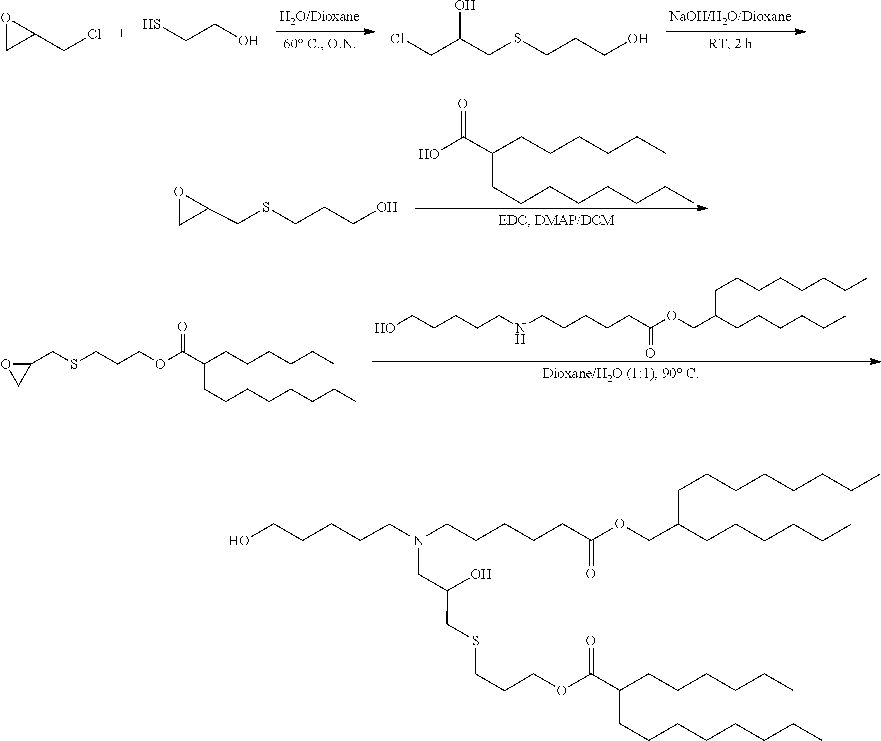

(±)-epichlorohydrin (2.04, 22 mmol) and 3-mercaptopropanol (1.84 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature. 100 mL H₂O was added, the mixture was extracted with DCM (50 mL×3). DCM layers were combined, dried over anhydrous Na₂SO₄, and DCM was removed under vacuum to afford 3-((oxiran-2-ylmethyl)thio)propan-1-ol as colorless oil (1.82 g, 70%). The crude product was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 3-((oxiran-2-ylmethyl)thio)propan-1-ol (1.48 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford 3-((oxiran-2-ylmethyl)thio)propyl 2-hexyldecanoate as colorless oil (2.12 g, 54%). The crude product was used without further purification. 3-((oxiran-2-ylmethyl)thio)propyl 2-hexyldecanoate (232 mg, 0.6 mmol) and 6-((5-hydroxypentyl)amino)hexanoate (264 mg, 0.6 mmol) in a mixture of 1,4-dioxane and H₂O (v:v, 1:1, 1 mL) was heated to 90° C. and stirred for 36 hours. The solvents were removed under vacuum and the residue was purified silica get chromatography (mixture of 0.1% NH₄OH, 5% MeOH in DCM) to obtain the product as a light brown oil (225 mg, 45%). ¹H NMR (500 MHz CDCl₃) δ 4.15 (t, J=6.3 Hz, 2H), 3.95 (d, J=5.8 Hz, 2H), 3.73 (d, J=5.6 Hz, 1H), 3.62 (t, J=6.5 Hz, 2H), 2.68-2.60 (m, 3H), 2.58 (dd, J=8.3, 5.9 Hz, 2H), 2.56-2.46 (m, 4H), 2.46-2.33 (m, 4H), 2.29 (dd, J=9.7, 5.2 Hz, 3H), 1.91 (dt, J=13.8, 6.9 Hz, 2H), 1.60 (ddd, J=19.3, 14.0, 6.9 Hz, 8H), 1.44 (dd, J=15.1, 6.7 Hz, 6H), 1.37-1.15 (m, 46H), 0.91-0.80 (m, 12H).

### Example 60

### Synthesis of Compound 453

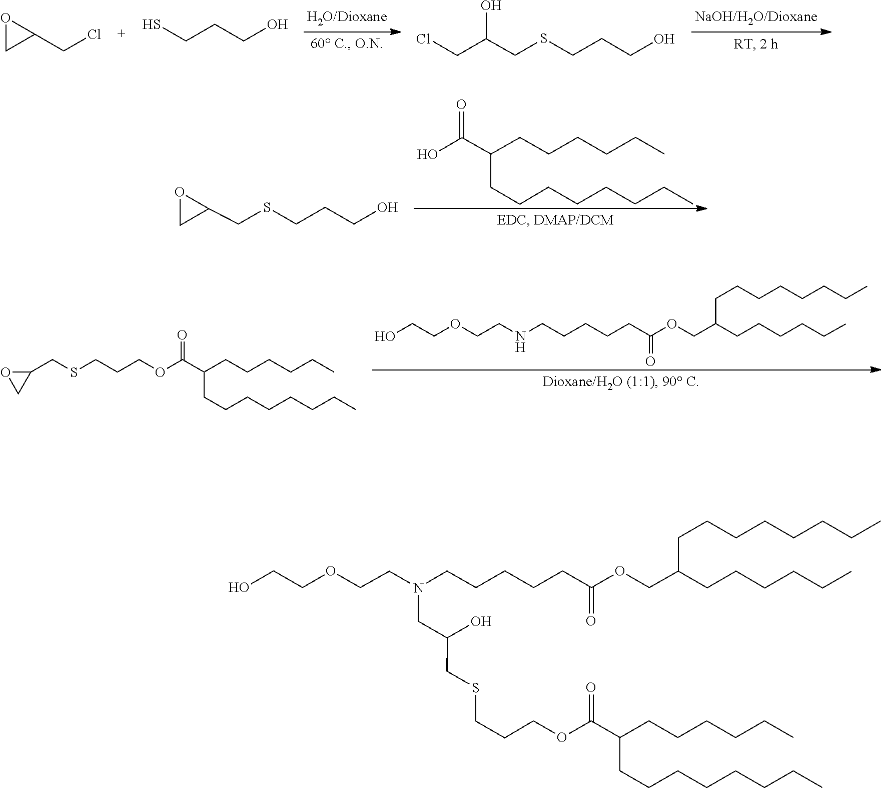

(±)-epichlorohydrin (2.04, 22 mmol) and 3-mercaptopropanol (1.84 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature. 100 mL H₂O was added, the mixture was extracted with DCM (50 mL×3). DCM layers were combined, dried over anhydrous Na₂SO₄, and DCM was removed under vacuum to afford 3-((oxiran-2-ylmethyl)thio)propan-1-ol as colorless oil (1.82 g, 70%). The crude product was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 3-((oxiran-2-ylmethyl)thio)propan-1-ol (1.48 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford 3-((oxiran-2-ylmethyl)thio)propyl 2-hexyldecanoate as colorless oil (2.12 g, 54%). The crude product was used without further purification.

3-((oxiran-2-ylmethyl)thio)propyl 2-hexyldecanoate (232 mg, 0.6 mmol) and 6-((2-(2-hydroxyethoxy)ethyl)amino)hexyl 2-hexyldecanoate (266 mg, 0.6 mmol) in a mixture of 1,4-dioxane and H₂O (v:v, 1:1, 1 mL) was heated to 90° C. and stirred for 36 hours. The solvents were removed under vacuum and the residue was purified silica get chromatography (mixture of 0.1% NH₄OH, 5% MeOH in DCM) to obtain the product as a light brown oil (218 mg, 43%). ¹H NMR (500 MHz, CDCl₃) δ 4.15 (t, J=6.3 Hz, 2H), 3.95 (d, J=5.8 Hz, 2H), 3.75 (s, 1H), 3.72-3.67 (m, 2H), 3.54 (dtd, J=30.2, 10.4, 4.4 Hz, 4H), 3.47 (t, J=0.9 Hz, 1H), 2.83-2.73 (m, 2H), 2.67-2.58 (m, 5H), 2.58-2.49 (m, 3H), 2.49-2.42 (m, 2H), 2.29 (t, J=7.4 Hz, 4H), 1.91 (dt, J=13.7, 6.8 Hz, 2H), 1.69-1.52 (m, 6H), 1.46 (dd, J=19.6, 12.2 Hz, 4H), 1.24 (d, J=5.3 Hz, 43H), 0.86 (ddd, J=7.1, 6.2, 3.0 Hz, 12H).

### Example 61

### Synthesis of Compound 454

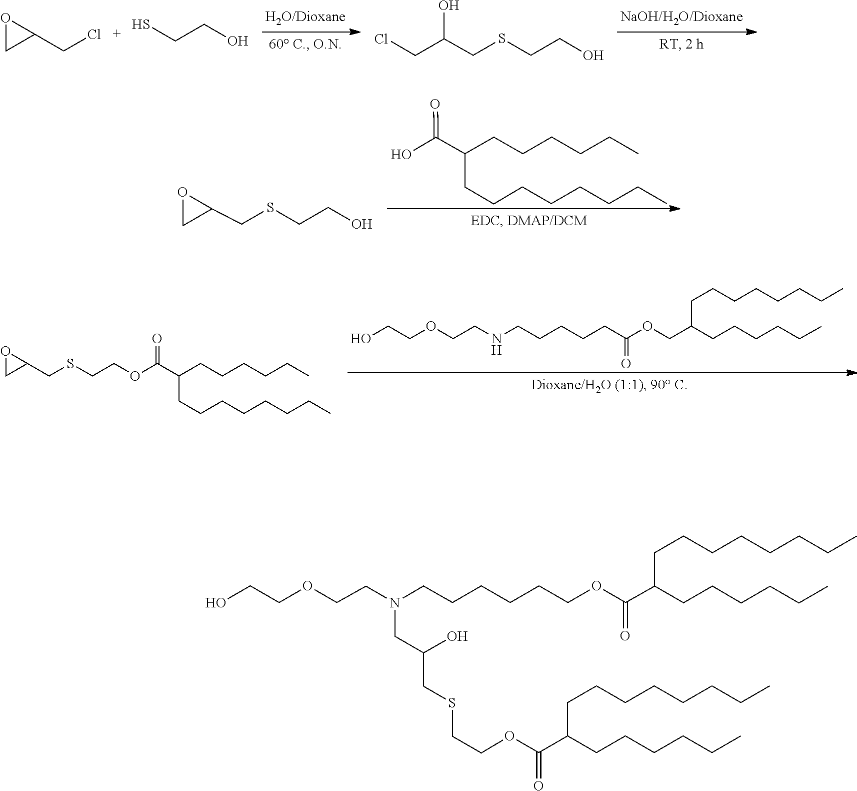

(±)-epichlorohydrin (2.04, 22 mmol) and 2-mercaptoethanol 1 (1.56 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature, then extracted with DCM (50 mL×3). DCM layers were combined, dried over anhydrous Na₂SO₄, and DCM was removed under vacuum to afford the crude product as colorless oil (1.82 g, 70%). The crude 2-(((oxiran-2-yl)methyl)thio)ethanol was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 2-(((oxiran-2-yl)methyl)thio)ethanol (1.34 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford the product as colorless oil (2.32 g, 62%). The crude 2-((oxiran-2-ylmethyl)thio)ethyl 2-hexyldecanoate was used without further purification.

2-((oxiran-2-ylmethyl)thio)ethyl 2-hexyldecanoate (222 mg, 0.6 mmol) and 6-((2-(2-hydroxyethoxy)ethyl)amino)hexyl 2-hexyldecanoate (266 mg, 0.6 mmol) in a mixture of 1,4-dioxane and H₂O (v:v, 1:1, 1 mL) was heated to 90° C. and stirred for 24 hours. The solvents were removed under vacuum and the residue was purified silica get chromatography (mixture of 0.1% NH₄OH, 5% MeOH in DCM) to obtain the product as a light brown oil (195 mg, 40%).

### Example 62

### Synthesis of Compound 457

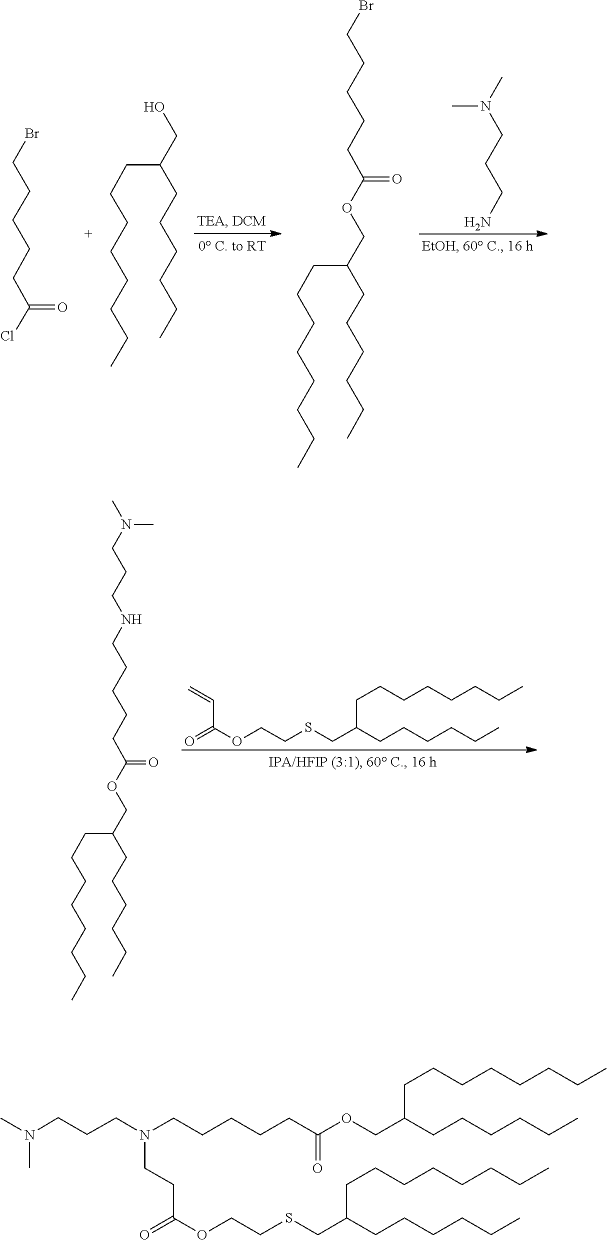

2-Hexyl-1-decanol (4.84 g, 20 mmol) and TEA (2.42 g, 24 mmol) was mixed in anhydrous DCM (20 mL) and cooled to 0° C. 6-Bromohexanoyl chloride (4.39 g, 2.06 mmol) was added slowly into the resulting mixture. The reaction mixture was stirred at 0° C. for 30 minutes and room temperature for an additional 3 hours. The solvent was removed under vacuum, and hexane (150 mL) was added to the residue. After washing with H₂O (50 mL×2) and acetonitrile (50 mL×2), the hexane layer was dried over anhydrous Na₂SO₄, and hexane was removed under vacuum to afford 2-hexyldecyl 6-bromohexanoate as colorless liquid (4.7 g, 56%).

A solution of 2-hexyldecyl 6-bromohexanoate (1.68 g, 4 mmol) and N,N-Dimethyl-1,3-propanediamine (4.09 g, 40 mmol) in ethanol (8 mL) was stirred at 60° C. for 16 hours. The solvent was removed under vacuum and the residue was re-suspended in ethyl acetate (100 mL). The suspension was washed with H₂O (50 mL×2), brine (50 mL×2), dried over anhydrous Na₂SO₄. The mixture was filtered, and the solvent was removed under vacuum to afford 2-hexyldecyl 6-((3-(dimethylamino)propyl)amino)hexanoate as colorless oil (1.22 g, 67%).

A solution of 2-hexyldecyl 6-((3-(dimethylamino)propyl)amino)hexanoate (440 mg, 1 mmol) and 2-((2-hexyldecyl)thio)ethyl acrylate (357 mg, 1 mmol) in a mixture of isopropanol/hexafluoroisopropanol (v:v, 3:1, 2 mL) was stirred at 60° C. for 16 hours. The solvents were removed, and the residue was purified by silica gel chromatography (mixture of 0.1% NH₄OH, 5% MeOH in DCM) to obtain the product as a light brown oil (425 mg, 53%). ¹H NMR (500 MHz, CDCl₃) δ 4.19 (t, J=7.0 Hz, 1H), 3.95 (d, J=5.9 Hz, 1H), 2.75 (t, J=6.9 Hz, 1H), 2.69 (t, J=7.0 Hz, 1H), 2.51 (d, J=6.3 Hz, 1H), 2.42 (t, J=6.8 Hz, 2H), 2.40-2.32 (m, 2H), 2.28 (d, J=11.2 Hz, 3H), 1.67-1.58 (m, 2H), 1.55-1.47 (m, 1H), 1.46-1.37 (m, 1H), 1.25 (s, 20H), 0.87 (t, J=6.9 Hz, 12H).

### Example 63

### Synthesis of Compound 458

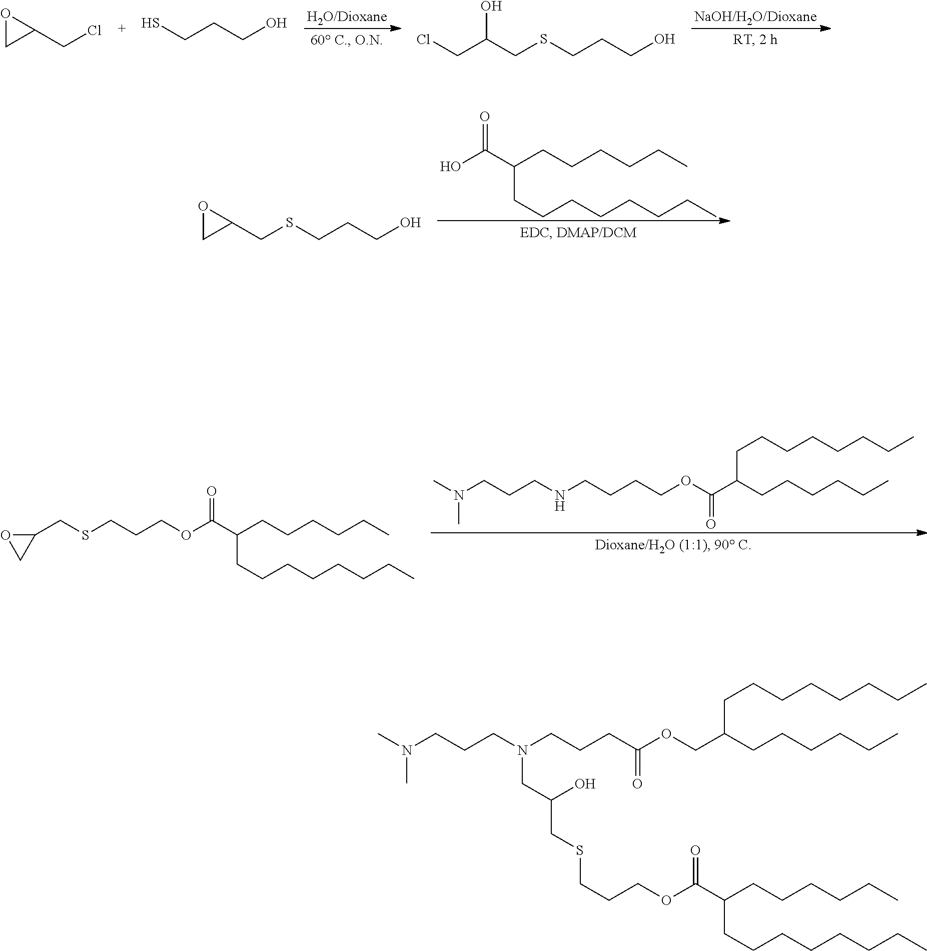

(±)-epichlorohydrin (2.04, 22 mmol) and 3-mercaptopropanol (1.84 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature. 100 mL H₂O was added, the mixture was extracted with DCM (50 mL×3). DCM layers were combined, dried over anhydrous Na₂SO₄, and DCM was removed under vacuum to afford 3-((oxiran-2-ylmethyl)thio)propan-1-ol as colorless oil (1.82 g, 70%). The crude product was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 3-((oxiran-2-ylmethyl)thio)propan-1-ol (1.48 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford 3-((oxiran-2-ylmethyl)thio)propyl 2-hexyldecanoate as colorless oil (2.12 g, 54%). The crude product was used without further purification. 3-((oxiran-2-ylmethyl)thio)propyl 2-hexyldecanoate (232 mg, 0.6 mmol) and 2-hexyldecyl 6-((3-(dimethylamino)propyl)amino)hexanoate (264 mg, 0.6 mmol) in a mixture of 1,4-dioxane and H₂O (v:v, 1:1, 1 mL) was heated to 90° C. and stirred for 36 hours. The solvents were removed under vacuum and the residue was purified silica get chromatography (mixture of 0.1% NH₄OH, 5% MeOH in DCM) to obtain the product as a light brown oil (156 mg, 32%).

### Example 64

### Synthesis of Compound 465

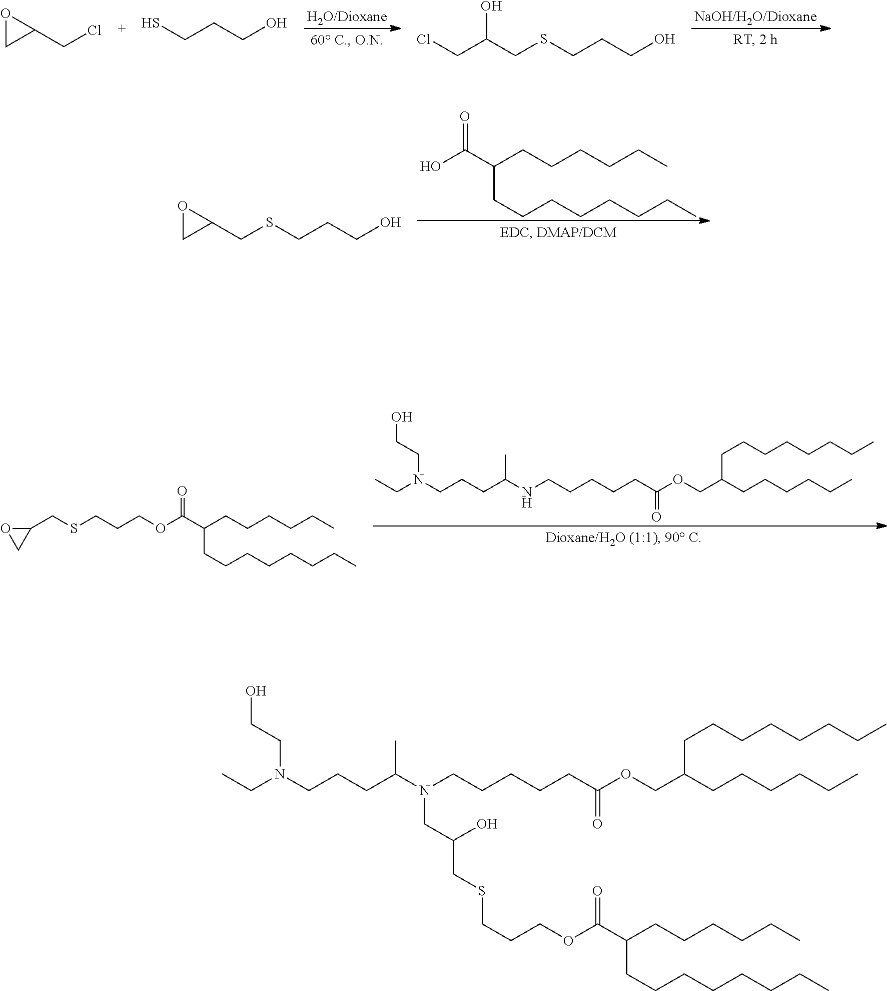

(±)-epichlorohydrin (2.04, 22 mmol) and 3-mercaptopropanol (1.84 g, 20 mmol) was stirred in a mixture of 1,4-dioxane and H₂O (v:v, 1:1, 10 mL) at 60° C. overnight. The reaction mixture was cooled to room temperature, and NaOH (880 mg, 22 mmol) in 25 mL H₂O was added slowly. The reaction mixture was stirred for 2 hours at room temperature. 100 mL H₂O was added, the mixture was extracted with DCM (50 mL×3). DCM layers were combined, dried over anhydrous Na₂SO₄, and DCM was removed under vacuum to afford 3-((oxiran-2-ylmethyl)thio)propan-1-ol as colorless oil (1.82 g, 70%). The crude product was used without further purification.

2-Hexyldecanoic acid (2.58 g, 10 mmol), EDC-HCl (2.30 g, 12 mmol) and DMAP (1.46 g, 12 mmol) was stirred in anhydrous DCM for 20 minutes at room temperature. 3-((oxiran-2-ylmethyl)thio)propan-1-ol (1.48 g, 10 mmol) was added into the resulting mixture and stirred for 24 hours. DCM was removed under vacuum. The residue was resuspended with 150 mL of hexane. The suspension was washed with H₂O (50 mL×2), brine (50 mL×2) and acetonitrile (50 mL×2). The hexane layer was separated, dried over anhydrous Na₂SO₄, and evaporated under vacuum to afford 3-((oxiran-2-ylmethyl)thio)propyl 2-hexyldecanoate as colorless oil (2.12 g, 54%). The crude product was used without further purification.

3-((oxiran-2-ylmethyl)thio)propyl 2-hexyldecanoate (232 mg, 0.6 mmol) and 2-hexyldecyl 6-((5-(ethyl(2-hydroxyethyl)amino)pentan-2-yl)amino)hexanoate (300 mg, 0.6 mmol) in a mixture of 1,4-dioxane and H₂O (v:v, 1:1, 1 mL) was heated to 90° C. and stirred for 36 hours. The solvents were removed under vacuum and the residue was purified silica get chromatography (mixture of 0.1% NH₄OH, 5% MeOH in DCM) to obtain the product as a light brown oil (220 mg, 41%). ¹H NMR (500 MHz, CDCl₃) δ 4.15 (s, 3H), 3.95 (d, J=5.6 Hz, 2H), 3.57 (s, 3H), 2.61 (d, J=17.3 Hz, 13H), 2.29 (d, J=7.1 Hz, 4H), 1.92 (s, 3H), 1.60 (s, 7H), 1.42 (s, 6H), 1.24 (d, J=5.3 Hz, 46H), 1.05 (s, 3H), 1.00 (d, J=6.5 Hz, 2H), 0.92 (d, J=6.4 Hz, 1H), 0.86 (dd, J=7.2, 2.8 Hz, 12H).

### Example 65

### Synthesis of Compound 468

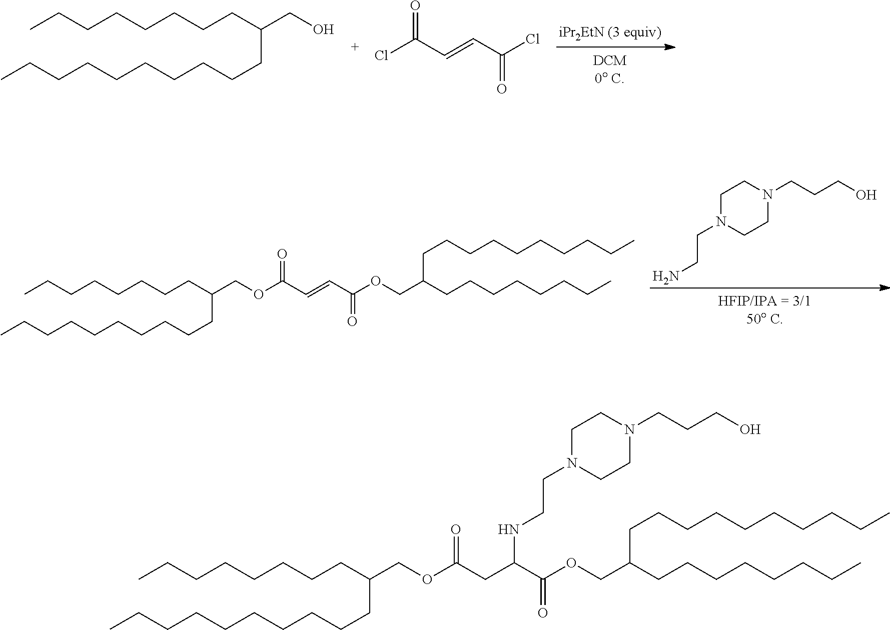

6.0 g 2-octyl-1-dodecanol (2 eq.) and 3.9 g diisopropyl ethyl amine (3 eq) were mixed with 20 mL anhydrous DCM in a 100 mL round bottom flask. The mixture was cooled to −0° C. with an ice bath. 1.8 g fumaryl chloride (1.2 eq) was added into the reaction mixture dropwise with vigorous stirring. The ice bath was then removed, and the temperature was allowed to rise to room temperature. The mixture was stirred overnight at room temperature. The reaction endpoint was confirmed by TLC when the 2-octyl-1-dodecanol was consumed. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane to yield di(2-octyldodecyl)fumarate in 85% yield (5.8 g).

2.0 g di(2-octyldodecyl) fumarate (1 eq.) and 830 mg 3-[4-(2-aminoethyl)-1-piperazinyl]-1-propanol (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 5% to 50% ethyl acetate in hexane. The product was collected as a pale-yellow oil (30% yield, 770 mg). ¹H NMR (500 MHz, CDCl₃) δ4.11 (qd, J=7.1, 1.4 Hz, 1H), 4.06-3.88 (m, 4H), 3.78 (t, J=5.2 Hz, 2H), 3.65 (t, J=6.5 Hz, 1H), 2.75 (ddd, J=27.2, 14.2, 6.3 Hz, 3H), 2.59 (ddq, J=16.6, 11.2, 6.3 Hz, 5H), 2.44 (ddt, J=45.0, 11.4, 6.2 Hz, 5H), 2.03 (d, J=1.4 Hz, 1H), 1.69 (t, J=5.7 Hz, 2H), 1.61 (s, 2H), 1.41 (d, J=1.3 Hz, 1H), 1.26 (d, J=7.1 Hz, 64H), 0.86 (qd, J=6.3, 1.4 Hz, 14H).

### Example 66

### Synthesis of Compound 469

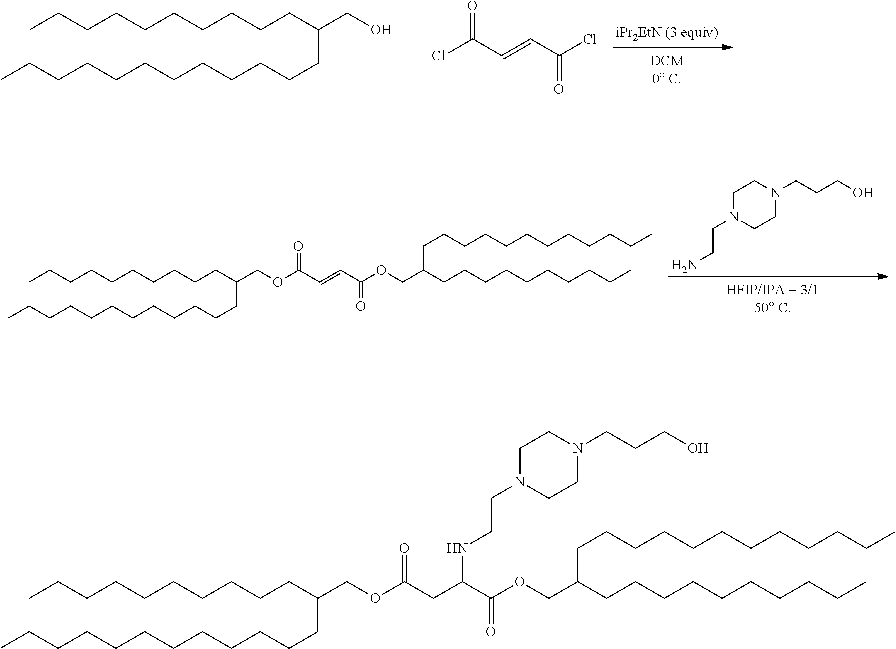

7.1 g 2-decyl-1-tetradecanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane to yield di(2-decyltetradecyl)fumarate in 85% yield (6.7 g).

2.3 g di(2-decyltetradecyl) fumarate (1 eq.) and 830 mg 3-[4-(2-aminoethyl)-1-piperazinyl]-1-propanol (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 5% to 50% ethyl acetate in hexane. The product was collected as a pale-yellow oil (30% yield, 870 mg). ¹H NMR (500 MHz, CDCl₃) δ 3.99 (dd, J=20.4, 5.8 Hz, 4H), 3.79 (t, J=5.2 Hz, 2H), 3.65 (t, J=6.6 Hz, 1H), 2.76 (dt, J=17.0, 6.1 Hz, 2H), 2.71 (d, J=5.8 Hz, 1H), 2.69-2.53 (m, 6H), 2.49 (dt, J=12.7, 6.5 Hz, 2H), 2.44-2.36 (m, 2H), 1.77-1.66 (m, 3H), 1.61 (s, 3H), 1.25 (s, 83H), 0.97-0.78 (m, 12H).

### Example 67

### Synthesis of Compound 476

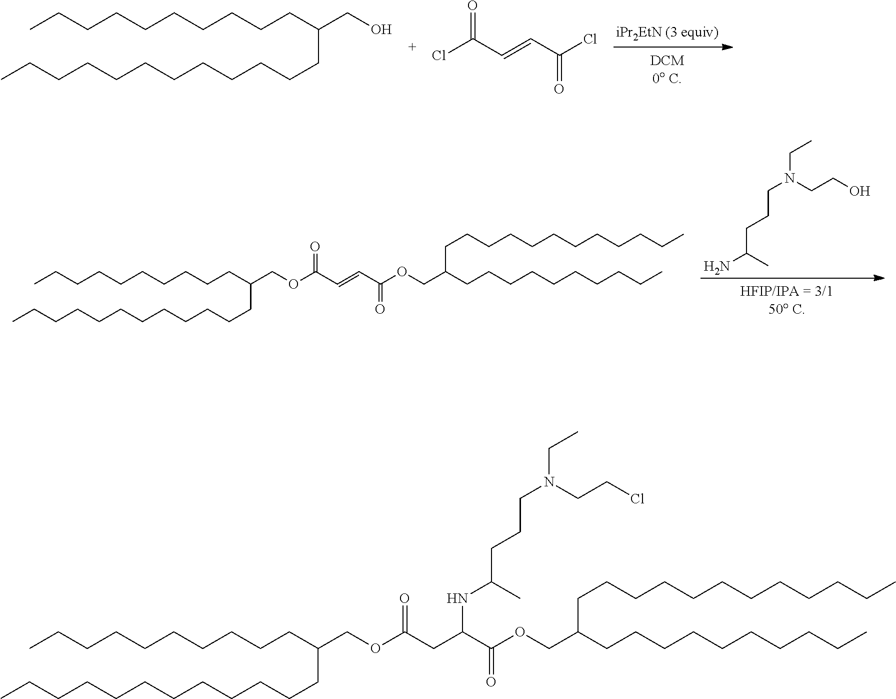

7.1 g 2-decyl-1-tetradecanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane to yield di(2-decyltetradecyl)fumarate in 85% yield (6.7 g).

2.3 g di(2-decyltetradecyl) fumarate (1 eq.) and 760 mg 2-((4-aminopentyl)(ethyl)amino)ethanol (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 5% to 50% ethyl acetate in hexane. The product was collected as a pale-yellow oil (30% yield, 840 mg). ¹H NMR (500 MHz, CDCl₃) δ 4.07-3.91 (m, 4H), 3.69 (dt, J=13.1, 6.4 Hz, 1H), 3.58-3.47 (m, 2H), 2.72-2.49 (m, 6H), 2.43 (t, J=7.2 Hz, 2H), 1.61 (s, 3H), 1.25 (s, 86H), 1.07-0.94 (m, 6H), 0.87 (t, J=6.9 Hz, 12H).

### Example 68

### Synthesis of Compound 477

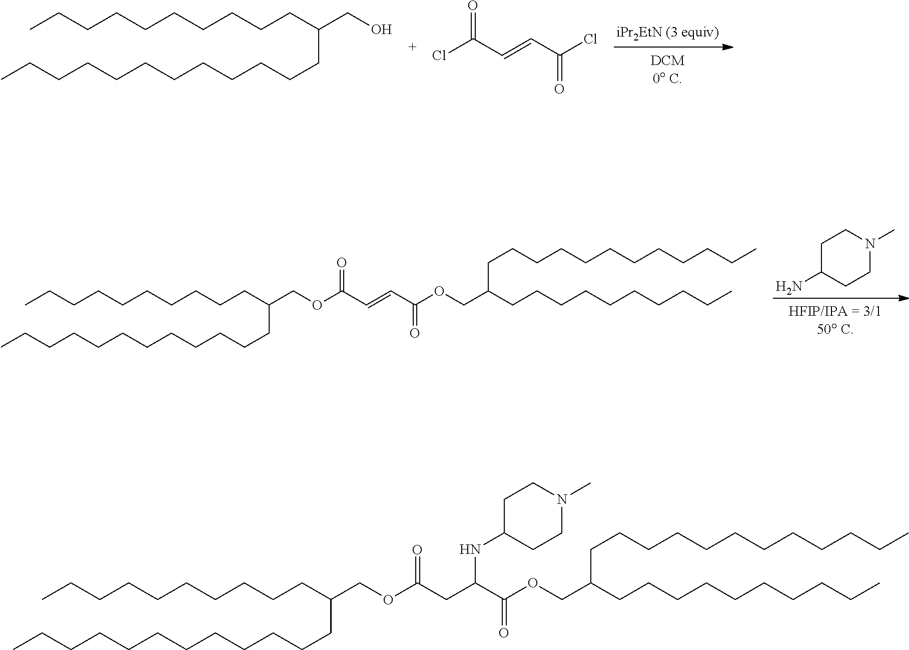

7.1 g 2-decyl-1-tetradecanol (2 eq.) and 3.9 g diisopropyl ethyl amine (3 eq) were mixed with 20 mL anhydrous DCM in a 100 mL round bottom flask. The mixture was cooled to −0° C. with an ice bath. Then 1.8 g fumaryl chloride (1.2 eq) was added into the reaction mixture dropwise with vigorous stirring. After adding the fumaryl chloride, the ice bath was removed, and the temperature was allowed to rise to room temperature. The mixture was stirred overnight at room temperature. The reaction endpoint was confirmed by TLC. The reaction mixture was diluted with 60 mL ethyl acetate and washed 3 times with 1M HCl aqueous solution, DI water, and saturated NaCl solution, subsequently. Then the organic layer was separated and dried by vacuum distillation. The residue was purified by silica column chromatography with hexane to yield di(2-decyltetradecyl)fumarate in 85% yield (6.7 g). 2.3 g di(2-decyltetradecyl) fumarate (1 eq.) and 500 mg 4-amino-1-methylpiperidine (1.5 eq) were mixed with 4.5 mL of IPA (isopropyl alcohol) and 1.5 mL of HFIPA (hexafluoro-2-propanol) in a 25 mL round bottom flask. The mixture was heated to 50° C. with an oil bath for ~6 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 5% to 50% ethyl acetate in hexane. The product was collected as a pale-yellow oil (25% yield, 660 mg).

### Example 69

### Synthesis of Compound 478

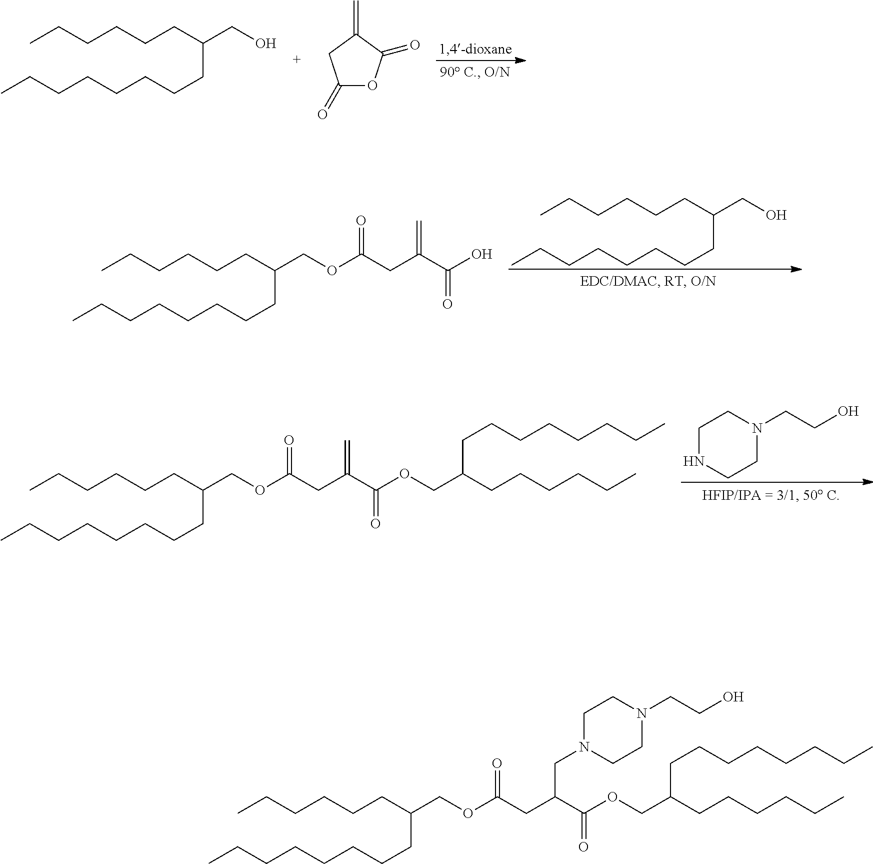

4.8 g 2-hexyl-1-decanol (1 eq.) and 2.35 itaconic anhydride (1.05 eq) were mixed with 10 mL dioxane in a 100 mL round bottom flask. After fully dissolved, this mixture was heated to 90° C. and stirred overnight. The reaction endpoint was confirmed by TLC when the starting materials were consumed. Then solvent was removed by vacuum distillation, and the residue was used without further purification as β-mono(2-hexyldecyl)itaconate (99% yield, 7.1 g). 5.0 g 2-hexyl-1-decanol (1.05 eq), 4.6 g 1-ethyl-3-(3-dimethylaminopropyl)carbodiimide hydrochloride (EDC, 1.2 eq) and 2.9 g 4-dimethylaminopyridine (DMAP, 1.2 eq) were mixed with β-mono(2-hexyldecyl)itaconate (7.1 g, 1 eq) and 30 mL of DCM in a 100 mL round bottom flask. The mixture immediately turned to deep red and was stirred overnight under room temperature. The reaction endpoint was confirmed by TLC when the starting materials were consumed. The reaction mixture was diluted with 70 mL ethyl acetate and washed 3 times with DI water and saturated NaCl solution, subsequently. Then the organic layer was separated and dried by vacuum distillation. The residue was purified by silica column chromatography with hexane to yield bis(2-hexyldecyl)itaconate (70% yield, 8.1 g).

1.0 g bis(2-hexyldecyl)itaconate (1 eq.) and 1.13 g N-(2-Hydroxyethyl)piperazine (5.0 eq) were mixed with 5 mL of IPA (isopropyl alcohol) in a 25 mL round bottom flask. The mixture was heated to 60° C. with an oil bath for overnight. The reaction was monitored by TLC and ended when the starting materials were consumed. Then the solvent was removed by vacuum distillation, to yield the deep red oily residue as the crude product. The crude product was purified by silica column chromatography, with a mobile phase composed of 0% to 25% ethyl acetate in hexane. The product was collected as a light red oil (30%, 370 mg). ¹H NMR (500 MHz, CDCl₃) δ 4.10 (qd, J=7.1, 1.4 Hz, 1H), 4.05-3.85 (m, 4H), 3.57 (t, J=5.4 Hz, 2H), 3.05 (pent, J=7.5 Hz, 1H), 2.70-2.52 (m, 3H), 2.44 (ddd, J=39.8, 11.8, 7.1 Hz, 8H), 2.09-1.97 (m, 2H), 1.59 (s, 2H), 1.40 (d, J=1.4 Hz, 1H), 1.36-1.08 (m, 47H), 0.85 (q, J=6.1 Hz, 12H).

### Example 70

### Synthesis of Compound 481

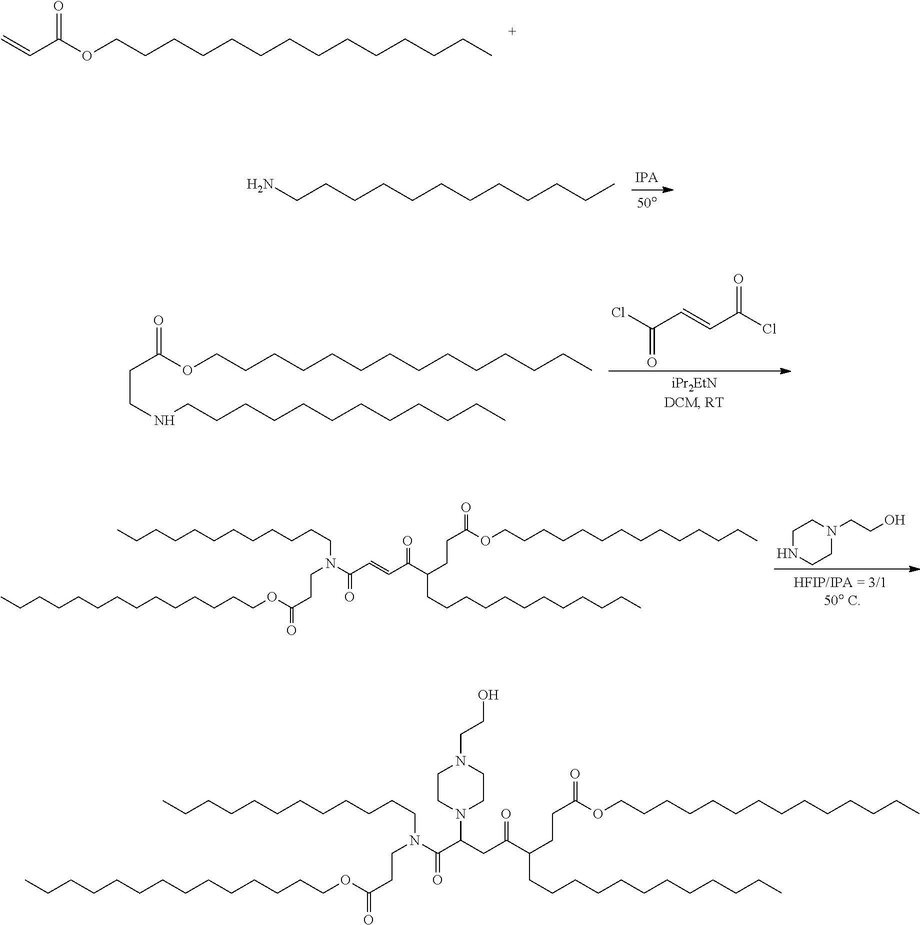

5.4 g tetradecyl acrylate (1 eq) and 5.6 g dodecyl amine (1.5 eq) were mixed with 10 mL of IPA (isopropyl alcohol) in a 50 mL round bottom flask. The mixture was heated to 60° C. with an oil bath for ~6 h. The reaction was monitored by TLC and ended when the starting materials were consumed. Then the solvent was removed by vacuum distillation, diluted with 50 mL of ethyl acetate, and washed 3 times with 20 mL of DI water and brine, subsequently. After the organic layer was separated and the solvent was removed by vacuum distillation, a yellow oily residue was yielded as the crude tetradecyl 3-(dodecylamino)propanoate. Then it was purified by silica column chromatography with a mobile phase composed of 10% ethyl acetate in hexane to yield tetradecyl 3-(dodecylamino)propanoate as a pale-yellow oil in 45% yield (4.1 g).

2.3 g tetradecyl 3-(dodecylamino)propanoate (2.0 eq) and 1.0 g diisopropyl ethyl amine (3 eq) were mixed with 10 mL anhydrous DCM in a 50 mL round bottom flask. The mixture was cooled to −0° C. with an ice bath. Then 460 mg fumaryl chloride (1.2 eq) was added into the reaction mixture dropwise with vigorous stirring. The ice bath was removed, and the temperature was allowed to rise to room temperature. The mixture was stirred overnight at room temperature. The reaction endpoint was confirmed by TLC. The reaction mixture was diluted with 25 mL ethyl acetate and washed 3 times with 1M HCl aqueous solution, DI water, and saturated brine, subsequently. Then the organic layer was separated and dried by vacuum distillation. The residue was purified by silica column chromatography with 10% ethyl acetate in hexane to yield diethyl 3,3′-{[(2E)-1,4-dioxobut-2-ene-1,4-diyl]bis(dodecylazanediyl)}tetradecanoate in 70% (1.8 g).

990 mg diethyl 3,3′-{[(2E)-1,4-dioxobut-2-ene-1,4-diyl]bis(dodecylazanediyl)}tetradecanoate (1 eq.) and 650 mg N-(2-hydroxyethyl)piperazine (5.0 eq) were mixed with 4.5 mL of IPA (isopropyl alcohol) and 1.5 mL of HFIPA (hexafluoro-2-propanol) in a 25 mL round bottom flask. The mixture was heated to 60 C with an oil bath for 24 h. The reaction was monitored by TLC and ended when the starting materials were consumed. Then the solvent was removed by vacuum distillation, to yield a yellow oily residue as the crude product. The crude product was purified by silica column chromatography, with a mobile phase composed of 0% to 33% ethyl acetate in hexane. The product was collected as a yellow oil in 25% yield (280 mg). ¹H NMR (500 MHz, CDCl₃) δ ¹H NMR (500 MHz, CDCl₃) δ 4.40 (pent, J=6.2 Hz, 1H), 4.21-3.97 (m, 5H), 3.97-3.79 (m, 1H), 3.79-3.46 (m, 6H), 3.41 (dd, J=13.8, 7.2 Hz, 2H), 3.33-3.12 (m, 2H), 3.01 (ddd, J=25.3, 15.8, 10.4 Hz, 2H), 2.79 (t, J=8.0 Hz, 1H), 2.67-2.40 (m, 11H), 2.40-2.26 (m, 1H), 1.77-1.51 (m, 7H), 1.44 (d, J=21.1 Hz, 2H), 1.39-1.06 (m, 80H), 0.87 (t, J=6.8 Hz, 12H).

### Example 71

### Formulation of Lipid Nanoparticles Encapsulating mRNA

A 2.7 mL ethanolic solution of cholesterol, DSPC, DMG-PEG, and an ionizable lipid was prepared by dissolving the lipids at 60° C. then cooling to room temperature. The solution was then loaded into a syringe and set into a syringe pump set to 2.5 mL/min. An 8.0 mL solution of 11.2 g/mL solution of firefly luciferase mRNA (TriLink BioTechnologies) in 25 mM acetate buffer (pH 5.10) was loaded into a second syringe and set into a syringe pump set to 7.5 mL/min. The syringes were then affixed to tubing leading to a chaotic mixer microfluidic device and set into syringe pumps with predetermined flow rates. The tubing was primed with the respective solutions and the syringe pumps were turned on to allow mixing. The product was diluted with water then the sample was loaded into a dialysis tube and dialyzed against Tris buffer in 10% sucrose solution (pH 7.0) at 2-8° C. for 24 hrs. The LNP sample was then transferred to a 30 kDa MWCO centrifugal filter device and concentrated to 1 mL.

TABLE 1
Representative lipid nanoparticle compositions

| Lipid Composition | N:P | Particle Size | | Encapsulation |
|---|---|---|---|---|
| (mol ratios: 50:38.5:10:1.5) | Ratio | (nm) | PDI | Efficiency |
| Compound 83:cholesterol:DSPC:DMG-PEG | 6 | 275.8 | 0.186 | 67.60% |
| Compound 84:cholesterol:DSPC:DMG-PEG | 6 | 162.5 | 0.256 | 89.00% |
| Compound 135:cholesterol:DSPC:DMG-PEG | 6 | 128.8 | 0.180 | 95.63% |
| Compound 198:cholesterol:DSPC:DMG-PEG | 6 | 184.8 | 0.203 | 80.00% |
| Compound 226:cholesterol:DSPC:DMG-PEG | 6 | 152.6 | 0.175 | 75.92% |
| Compound 227:cholesterol:DSPC:DMG-PEG | 6 | 181.2 | 0.172 | 98.94% |
| Compound 228:cholesterol:DSPC:DMG-PEG | 6 | 137.0 | 0.145 | 98.25% |
| Compound 247:cholesterol:DSPC:DMG-PEG | 6 | 138.2 | 0.093 | 90.93% |
| Compound 253:cholesterol:DSPC:DMG-PEG | 6 | 113.5 | 0.137 | 94.72% |
| Compound 255:cholesterol:DSPC:DMG-PEG | 6 | 148.1 | 0.116 | 94.92% |
| Compound 256:cholesterol:DSPC:DMG-PEG | 6 | 117.5 | 0.141 | 94.77% |
| Compound 355:cholesterol:DSPC:DMG-PEG | 6 | 149.7 | 0.168 | 97.30% |
| Compound 356:cholesterol:DSPC:DMG-PEG | 6 | 177.6 | 0.171 | 97.94% |
| Compound 359:cholesterol:DSPC:DMG-PEG | 6 | 202.3 | 0.147 | 98.26% |
| Compound 374:cholesterol:DSPC:DMG-PEG | 6 | 180.3 | 0.145 | 98.09% |
| Compound 375:cholesterol:DSPC:DMG-PEG | 6 | 166.1 | 0.172 | 98.64% |
| Compound 441:cholesterol:DSPC:DMG-PEG | 6 | 196.2 | 0.171 | 94.18% |
| Compound 443:cholesterol:DSPC:DMG-PEG | 6 | 132.6 | 0.111 | 97.29% |
| Compound 444:cholesterol:DSPC:DMG-PEG | 6 | 165.8 | 0.085 | 98.19% |
| Compound 445:cholesterol:DSPC:DMG-PEG | 6 | 119.0 | 0.107 | 95.35% |
| Compound 468:cholesterol:DSPC:DMG-PEG | 6 | 179.9 | 0.148 | 95.22% |

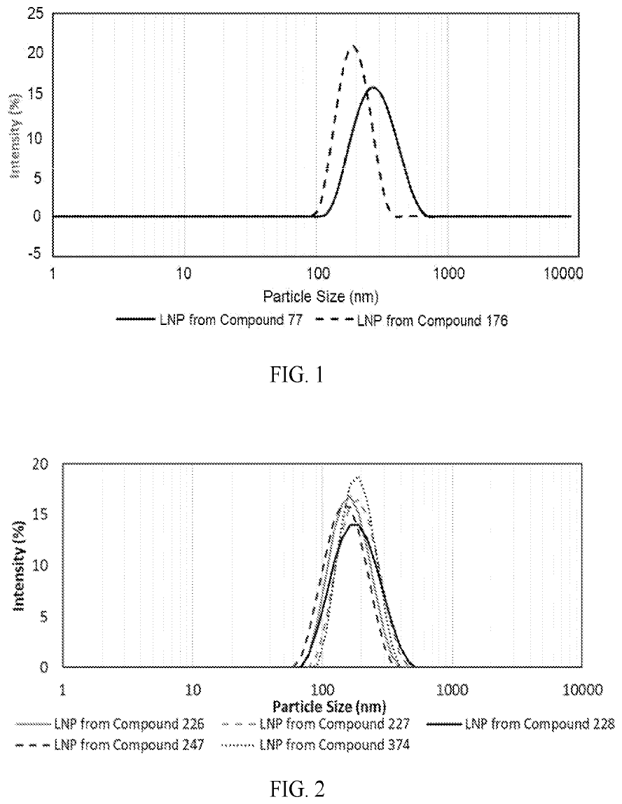

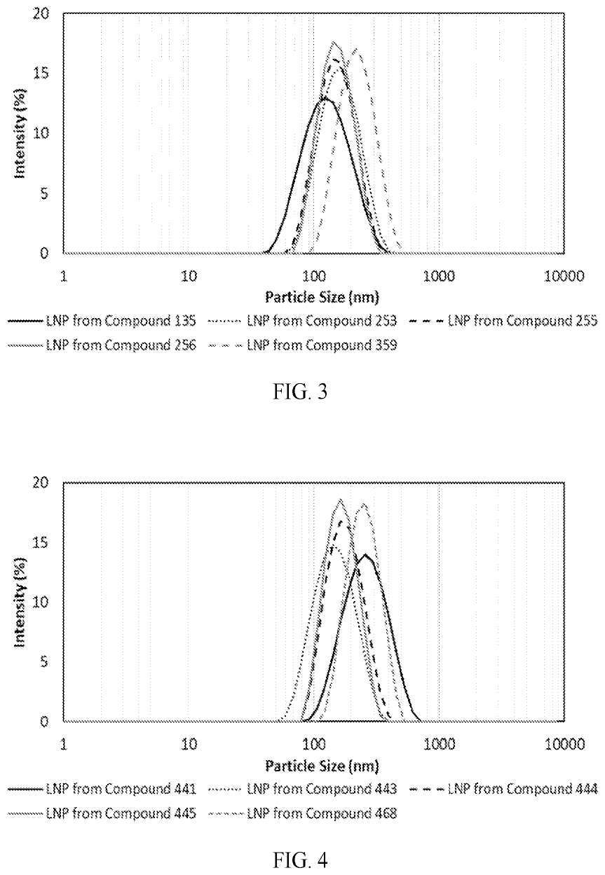

Representative lipid nanoparticle compositions and characterizations are shown in Table 1 above. The total and encapsulated mRNA was determined by Ribogreen ™ assay. Particle size and size distribution were characterized by dynamic light scattering, see FIG. 1, FIG. 2, FIG. 3, and FIG. 4.

### Example 72

### Luciferase mRNA Transfection of HEK-293 Cells and Luminescence Quantification

Eagle's Minimum Essential Medium (EMEM) cell culture media was used for the culture of HEK-293 cells. To seed cells on a 24-well plate, the cell suspension was prepared as 250,000 cells/mL, and then 500 μL of this suspension was pipetted into each well. The seeded plate was then incubated for 1 day at 37° C. before use. The next day, the cell culture media was replaced with 400 μL of transfection medium (EMEM culture media plus fetal bovine serum). The well plate was then loaded with 100 μL of the samples (e.g., encapsulated mRNA within lipid nanoparticle formulation, or a negative control, i.e., unencapsulated mRNA). The well plate was incubated at 37° C. for 24 hours for mRNA transfection.

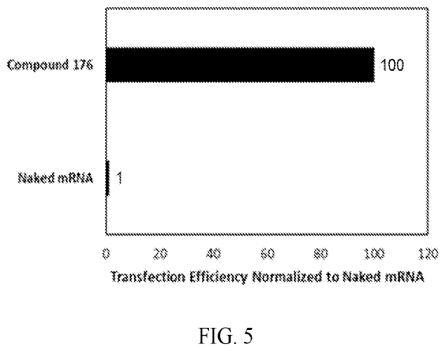

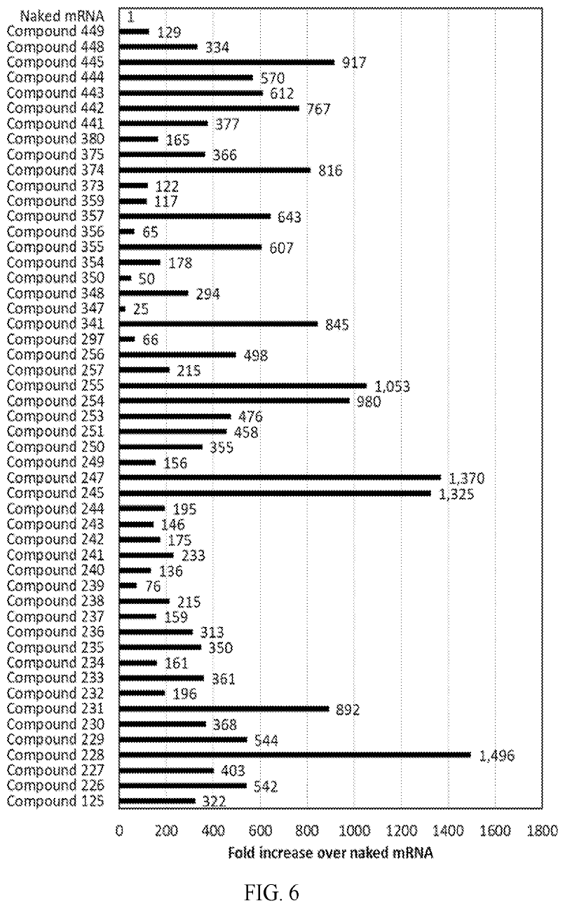

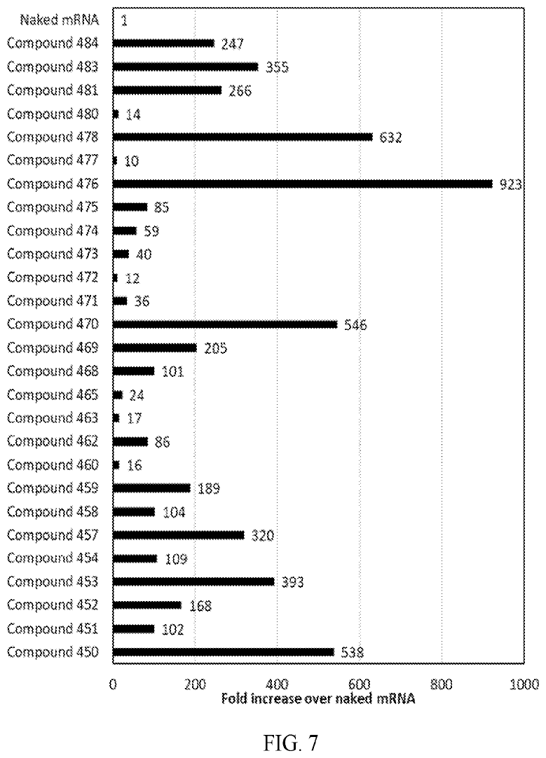

The next day, cells were washed and lysed using 200 μL of 1× cell lysis buffer plus one freeze-thaw cycle at −80° C. Then 20 μL of the lysate was mixed with 100 μL of luciferase reporter reagent, and the luminescence was read on a luminometer (ThermoFisher). Lysate protein content was measured using a BCA protein assay kit. The luminescence for each sample was then normalized to its corresponding protein content. A representative sample of the in vitro performance can be seen in FIG. 5, FIG. 6, FIG. and 7.

The foregoing embodiments and examples are provided for illustrative purposes only and are not intended to limit the scope of the invention. Many variations to those described above may be possible. Since various modifications and variations to the embodiments and examples described above will be apparent to those of skill in this art based on the present disclosure, such modifications and variations are within the spirit and scope of the present invention. All patent or non-patent literature cited are incorporated herein by reference in their entireties without admission of them as prior art.

1. A compound of Formula I, II, III, IV, V, VI, VII, VIII, IX, X, XI, XII, XIII, or XIV: 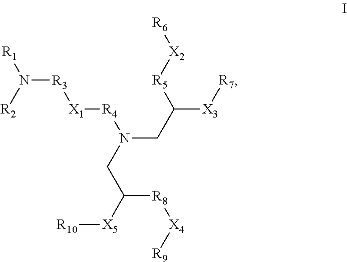
or an isomer, or a salt thereof, wherein:
R₁ and R₂ are the same or different, each independently alkyl, alkenyl, or alkynyl; or alternatively R₁ and R₂ together with the nitrogen atom to which they are attached form a heterocyclyl;
R₃ is a C₁₋₁₈ alkylene, C₂₋₁₈ alkenylene, or C₂₋₁₈ alkynylene, or together with the adjacent nitrogen atom forms a ring structure comprising 3-18 carbon atoms;
R₄ is a C₁₋₁₈ alkylene, C₂₋₁₈ alkenylene, or C₂₋₁₈ alkynylene;
R₅ and R₈ are the same or different, each independently a bond, or a C₁₋₂₈ alkylene, C₂₋₂₈ alkenylene, or C₂₋₂₈ alkynylene;
R₆ and R₉ are the same or different, each independently hydrogen or a linear C₁₋₂₈ alkyl or C₂₋₂₈ alkenyl;
R₇ and R₁₀ are the same or different, each independently hydrogen or a linear C₁₋₂₈ alkyl or C₂₋₂₈ alkenyl;
X₁ is a bond, —O—, —CO—, —OC—O—, or —O—CO—;
X₂ and X₄ are the same or different, each independently methylene (—CH₂—), —S—, —S—S—, —O—, —O—CO—, —CO—O—, or —NR—, wherein R is a lower alkyl; and
X₃ and X₅ are the same or different, each independently a methylene (—CH₂—), —S—, —S—S—, —O—, —O—CO—, —CO—O—, or —NR—, wherein R is a lower alkyl,
wherein the longest chain of atoms in the compound is between 18 to 70 atoms; 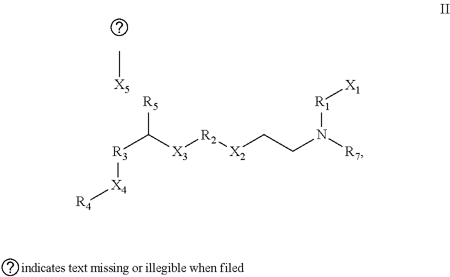
or an isomer, or a salt thereof, wherein:
R₁ is a C₁₋₁₈ alkylene, C₂₋₁₈ alkenylene, or C₂₋₁₈ alkynylene;
R₂ is a C₁₋₁₈ alkylene, C₂₋₁₈ alkenylene, or C₂₋₁₈ alkynylene, C₃₋₈ carbocyclylene, 3- to 8-membered heterocyclylene, or C₁₋₁₈ heteroalkylene;
R₃ and R₅ are the same or different, each independently a bond or a C₁₋₂₈ alkylene, C₂₋₂₈ alkenylene, or C₂₋₂₈ alkynylene;
R₄, R₆, and R₇ are the same or different, each independently hydrogen or a C₁₋₂₈ alkyl, C₂₋₂₈ alkenyl, or C₂₋₂₈ alkynyl, each optionally substituted by one, two, or three substituents independently selected from —OR, —SR, —S—S—R, —O—CO—R, —CO—OR, —CO—NR^{a}R^{b}, —NR^{a}—CO—R, —O—CO—NR^{a}R^{b}, —NR^{a}—CO—OR, —NR^{a}R^{b}, and —S—CO—R, wherein R at each occurrence is independently hydrogen or a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl; and R^{a} and R^{b} are independently hydrogen or lower alkyl;
X₁ is —OH, —SH, —N(R)₂, C₅₋₈ carbocyclyl, heterocyclyl, or hydrogen, or absent, wherein R at each occurrence is independently a lower alkyl or hydrogen;
X₂ is —O—CO—, —CO—O—, —NR—CO—, or —CO—NR—, wherein R is a lower alkyl or hydrogen; and
X₃, X₄, and X₅ are each independently —O—CO—, —CO—O—, —NR—CO—, —CO—NR—, a bond, or absent, wherein R is a lower alkyl or hydrogen,
wherein the longest chain of atoms in the compound is between 18 and 70 atoms; 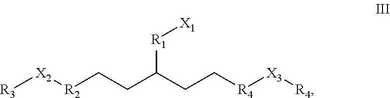
or an isomer, or a salt thereof, wherein:
R₁ is an optionally substituted C₁₋₁₈ alkylene, C₂₋₁₈ alkenylene, or C₂₋₈ alkynylene;
R₂ and R₄ are the same or different, each independently a bond or a C₁₋₂₈ alkylene, C₂₋₂₈ alkenylene, or C₂₋₂₈ alkynylene;
R₃ and R₅ are the same or different, each independently hydrogen or a C₁₋₂₈ alkyl, C₂₋₂₈ alkenyl, or C₂₋₂₈ alkynyl;
X₁ is —OH, —OR, —CO—OR, —CO—R, —O—CO—R, —SH, —SR, —N(R)₂, a C₅₋₈ carbocyclyl, a heterocyclyl, hydrogen, or absent, wherein R at each occurrence is independently hydrogen, or an optionally substituted methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl; and
X₂ and X₃ are the same or different, each independently —O—CO—, —CO—O—, —CO—, —NR—CO—, —CO—NR—, a bond, or absent, wherein R is a lower alkyl or hydrogen,
wherein the longest chain of atoms in the compound is between 18 to 70 atoms; 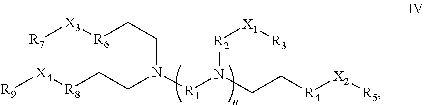
or an isomer, or a salt thereof, wherein:
R₁ is a C₁₋₁₈ alkylene, C₂₋₁₈ alkenylene, or C₂₋₁₈ alkynylene, each optionally substituted by one, two, or three substituents independently selected from —OR, —SR, —S—S—R, —O—CO—R, —CO—OR, —CO—NR^{a}R^{b}, —NR^{a}—CO—R, —O—CO—NR^{a}R^{b}, —NR^{a}—CO—OR, —NR^{a}R^{b}, and —S—CO—R,
wherein R at each occurrence is independently hydrogen or lower alkyl; and R^{a} and R^{b} are independently hydrogen or lower alkyl;
n is 0, 1, 2, 3, 4, 5, 6, 7, 8, 9, or 10;
R₂ is a C₈ alkylene, C₂₋₁₈ alkenylene, or C₂₋₁₈ alkynylene;
R₃ is a C₈ alkyl, C₂₋₁₈ alkenyl, or C₂₋₁₈ alkynyl, or a C₅₋₈ carbocyclyl, heterocyclyl, hydrogen, or absent;
R₄, R₆, and R₈ are the same or different, each independently a bond or a C₁₋₂₈ alkylene, C₂₋₂₈ alkenylene, or C₂₋₂₈ alkynylene;
R₅, R₇, and R₉ are the same or different, each independently a bond or a C₂₋₂₈ alkyl, C₂₋₂₈ alkenyl, or C₂₋₂₈ alkynyl;
X₁ is a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈carbocyclylene, a heterocyclylene, a bond, or absent; and
X₂, X₃, and X₄ are the same or different, each independently a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent,
wherein the longest chain of atoms in the compound is between 18 and 70 atoms; 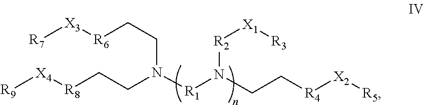
or an isomer, or a salt thereof, wherein:
R₁ is a C₈ alkylene, C₂₋₁₈ alkenylene, or C₂₋₁₈ alkynylene, each optionally substituted by one, two, or three substituents independently selected from —OR, —SR, —S—S—R, —O—CO—R, —CO—OR, —CO—NR^{a}R^{b}, —NR^{a}—CO—R, —O—CO—NR^{a}R^{b}, —NR^{a}—CO—OR, —NR^{a}R^{b}, and —S—CO—R, wherein R at each occurrence is independently hydrogen or lower alkyl; and R^{a} and R^{b} are independently hydrogen or lower alkyl;
n is 0, 1, 2, 3, 4, 5, 6, 7, 8, 9, or 10;
R₂ is a C₁₋₁₈ alkylene, C₂₋₁₈ alkenylene, or C₂₋₁₈ alkynylene;
R₃ is a C₈ alkyl, C₂₋₁₈ alkenyl, or C₂₋₁₈ alkynyl, or a C₅₋₈ carbocyclyl, heterocyclyl, hydrogen, or absent;
R₄, R₆, and R₈ are the same or different, each independently a bond or a C₁₋₂₈ alkylene, C₂₋₂₈ alkenylene, or C₂₋₂₈ alkynylene;
R₅, R₇, and R₉ are the same or different, each independently a bond or a C₂₋₂₈ alkyl, C₂₋₂₈ alkenyl, or C₂₋₂₈ alkynyl;
X₁ is a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent; and
X₂, X₃, and X₄ are the same or different, each independently a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent,
wherein the longest chain of atoms in the compound is between 18 and 70 atoms; 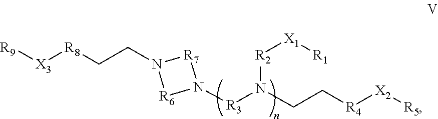
or an isomer, or a salt thereof, wherein:
R₁ is a C₂₋₁₈ alkyl, C₂₋₁₈ alkenyl, or C₂₋₈ alkynyl, a C₅₋₈ carbocyclyl, a heterocyclyl, hydrogen, or absent;
R₂ is a C₁₋₁₈ alkylene, C₂₋₁₈ alkenylene, or C₅₋₈ alkynylene;
R₃ is a C₁₋₁₈ alkylene, C₂₋₁₈ alkenylene, or C₅₋₈ alkynylene, each optionally substituted by one, two, or three substituents independently selected from —OR, —SR, —S—S—R, —O—CO—R, —CO—OR, —CO—NR^{a}R^{b}, —NR^{a}—CO—R, —O—CO—NR^{a}R^{b}, —NR^{a}—CO—OR, —NR^{a}R^{b}, and —S—CO—R, wherein R at each occurrence is independently hydrogen or lower alkyl; and R^{a} and R^{b} are independently hydrogen or lower alkyl;
n is 0, 1, 2, 3, 4, 5, 6, 7, 8, 9, or 10;
R₄ and R₈ are the same or different, each independently a bond or a C₁₋₂₈ alkylene C₂₋₂₈ alkenylene, or C₂₋₂₈ alkynylene;
R₅ and R₉ are the same or different, each independently hydrogen or a C₁₋₂₈ alkyl, C₂₋₂₈ alkenyl, or C₂₋₂₈ alkynyl;
R₆ and R₇ are the same or different, each independently a bond or a C₁₋₈ alkylene C₂₋₁₈ alkenylene, or C₂₋₁₈ alkynylene;
X₁ is a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅₋₈ carbocyclylene, a heterocyclylene, a bond, or absent; and
X₂, and X₃ are the same or different, each independently a methylene (—CH₂—), —O—, —S—, —S—S—, —NR— in which R is a lower alkyl, —CO—NR— in which R is a lower alkyl, —NR—CO— in which R is a lower alkyl or hydrogen, —O—CO—, —CO—O—, —CO—, —O—CO—O—, a C₅-s carbocyclylene, a heterocyclylene, a bond, or absent,
wherein the longest chain of atoms in the compound is between 18 to 70 atoms; 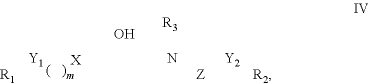
or an isomer, or a salt thereof, wherein:
Y₁ and Y₂ are the same or different, each independently selected from —(C═O)O—, —O(C═O)—, —(C═O)S—, —S(C═O)—, —NR(C═O)—, —(C═O)NR—, —O—, —S—, —S—S—, —CH₂—, an alkenylene, and an alkynylene, wherein R is independently a hydrogen, or an alkyl, alkenyl, alkynyl, carbocyclyl, heterocyclyl, group;
X is selected from —S—, —S—S—, —O—, —CH₂—, alkenylene, and alkynylene, —NR—, wherein R is hydrogen or an alkyl, alkenyl, alkynyl, carbocyclyl, heterocyclyl, group;
R₁ and R₂ are the same or different, each independently selected from carbocyclyl, alkyl, alkenyl, and alkynyl, each optionally substituted;
R₃ is selected from hydrogen, halogen, alkyl, alkenyl, alkynyl, carbocyclyl, heterocyclyl, aryl, and heteroaryl, each, except hydrogen and halogen, optionally substituted;
Z is selected from alkylene, alkenylene, alkynylene, and carbocyclylene; and
m is an integer selected from 1 to 24,
wherein the longest chain of atoms in the compound is between 18 to 70 atoms; 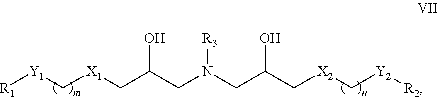
or an isomer, or a salt thereof, wherein:
Y₁ and Y₂ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, and alkynylene, wherein R at each occurrence is independently hydrogen, or an alkyl, alkenyl, alkynyl, carbocyclyl, heterocyclyl, group;
X₁ and X₂ are the same or different and are independently selected from —S—, —S—S—, —O—, —CH₂—, alkenylene, alkynylene, and —NR—, wherein R is hydrogen, or an alkyl, alkenyl, alkynyl, carbocyclyl, heterocyclyl, group;
R₁ and R₂ are the same or different and independently selected from alkyl, alkenyl, alkynyl, and heterocyclyl, each optionally substituted;
R₃ is selected from hydrogen, halogen, alkyl, alkenyl, alkynyl, carbocyclyl, heterocyclyl, aryl, and heteroaryl groups, each optionally substituted; and
m and n are each independently an integer selected from 1 to 24,
wherein the longest chain of atoms in the compound is between 18 to 70 atoms; 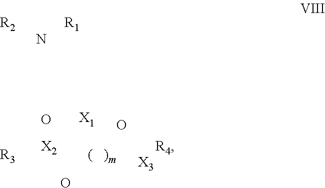
or an isomer, or a salt thereof, wherein:
X₁, X₂, and X₃ are independently selected from —CH₂—, —O—, —S—, and —NR—, wherein R is hydrogen or an alkyl, alkenyl, alkynyl, carbocyclyl, heterocyclyl, group;
R₁ and R₂ are the same or different and are independently selected from hydrogen, halogen, alkyl, alkenyl, alkynyl, carbocyclyl, heterocyclyl, aryl, and heteroaryl groups, each, except hydrogen and halogen, optionally substituted;
R₃ and R₄ are selected from carbocyclyl, alkyl, alkenyl, and alkynyl, each optionally substituted; and
m is 0, 1, 2, 3, 4, or 5,
wherein the longest chain of atoms in the compound is between 18 and 70 atoms; 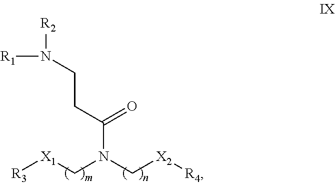
or an isomer, or a salt thereof, wherein:
X₁ and X₂ are the same or different moieties selected from —(C═O)O—, —O(C═O)—, —(C═O)S—, —S(C═O)—, —NR(C═O)—, —(C═O)NR—, —O—, —S—, —S—S—, —CH₂—, an alkene, and an alkyne, wherein R at each occurrence is independently hydrogen, or an alkyl, alkenyl, alkynyl, carbocyclyl, heterocyclyl group;
R₁ and R₂ are the same or different and are independently selected from hydrogen, halogen, alkyl, alkenyl, alkynyl, carbocyclyl, heterocyclyl, aryl, and heteroaryl groups, each except hydrogen and halogen optionally substituted;
R₃ and R₄ are the same or different and are independently selected from carbocyclyl, alkyl, alkenyl, and alkynyl; and
m and n are the same or different and are each an integer selected from 1 to 24,
wherein the longest chain of atoms in the compound is between 18 and 70 atoms; 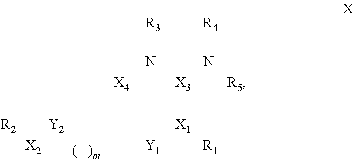
or an isomer, or a salt thereof, wherein:
R₁, R₂, R₃, R₄ and R₅ are the same or different and are independently selected from hydrogen, halogen, alkyl, alkenyl, alkynyl, carbocyclyl, heterocyclyl, aryl, and heteroaryl groups, each, except hydrogen and halogen, optionally substituted;
X₁, X₂, and X₃ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, and alkynylene, wherein R at each occurrence is independently hydrogen, or an alkyl, alkenyl, alkynyl, carbocyclyl, heterocyclyl, group;
X₄ is selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, and alkynylene, wherein R at each occurrence is independently hydrogen, or an alkyl, alkenyl, alkynyl, carbocyclyl, heterocyclyl, group; or X₄ is a linear alkyl group comprising 0 to 10 methylene units;
Y₁ and Y₂ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, or alkynylene, and carbocyclylene, wherein R at each occurrence is independently hydrogen, alkyl, alkenyl, alkynyl, carbocyclyl, or heterocyclyl; and
m is 0, 1, 2, 3, 4, 5, 6, 7, 8, 9, or 10,
wherein the longest chain of atoms in the compound is between 18 and 70 atoms; 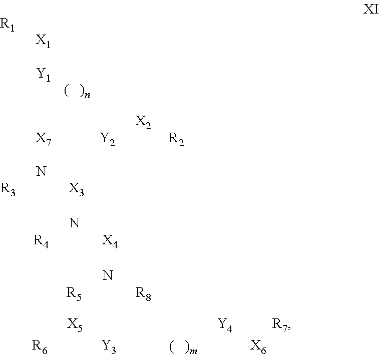
or an isomer, or a salt thereof, wherein:
R₁, R₂, R₃, R₄, R₅, R₆, and R₇ are the same or different and are independently selected from hydrogen, halogen, alkyl, alkenyl, alkynyl, carbocyclyl, heterocyclyl, aryl, and heteroaryl groups, each except hydrogen and halogen optionally substituted;
X₁, X₂, X₃, X₄, X₅ and X₆ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, alkynylenes, carbocyclyl, and a bond, wherein R at each occurrence is independently hydrogen, alkyl, alkenyl, alkynyl, carbocyclyl, or heterocyclyl;
X₇ and X₈ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, or alkynylene, and carbocycylyl, wherein R at each occurrence is independently hydrogen, alkyl, alkenyl, alkynyl, carbocyclyl, or heterocyclyl; or X₇ and X₈ are each independently a linear alkyl group comprising 0 to 10 methylene units;
Y₁, Y₂, Y₃, and Y₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, and an alkynylene wherein R at each occurrence is independently hydrogen, or an alkyl, alkenyl, alkynyl, carbocyclyl, heterocyclyl, group; and
n and m are independently selected from 0, 1, 2, 3, 4, 5, 6, 7, 8, 9, and 10,
wherein the longest chain of atoms in the compound is between 18 to 70 atoms; 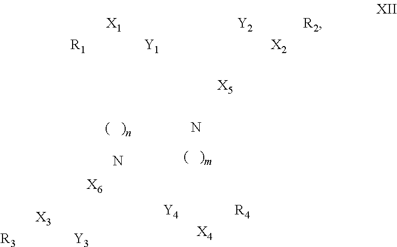
or an isomer, or a salt thereof, wherein:
R₁, R₂, R₃, and R₄ are the same or different and are independently selected from hydrogen, halogen, alkyl, alkenyl, alkynyl, carbocyclyl, heterocyclyl, aryl, and heteroaryl, each except hydrogen and halogen optionally substituted;
X₁, X₂, X, and X₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, alkynylene, and a bond, wherein R at each occurrent is independently hydrogen, alkyl, alkenyl, alkynyl, carbocyclyl, or heterocyclyl;
X₅ and X₆ are the same or different and are independently selected from —(C═O)O—, —O(C═O)—, —(C═O)S—, —S(C═O)—, —NR(C═O)—, —(C═O)NR—, —O—, —S—, —S—S—, —CH₂—NR—, alkenylene, alkynylene, and a bond, wherein R at each occurrence is independently hydrogen, or an alkyl, alkenyl, alkynyl, carbocyclyl, heterocyclyl, group; or X₅ and X₆ are each independently a linear alkyl group comprising 0 to 10 methylene units;
Y₁, Y₂, Y₃, and Y₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, alkynylene, and a bond, wherein R at each occurrence is independently hydrogen, or an alkyl, alkenyl, alkynyl, carbocyclyl, heterocyclyl, group;
the central ring structure is an optionally substituted aromatic, heteroaromatic, non-aromatic, or anti-aromatic ring system; and
n and m are independently selected from 0, 1, 2, 3, 4, 5, 6, 7, 8, 9, and 10,
wherein the longest chain of atoms in the compound is between 18 to 70 atoms; 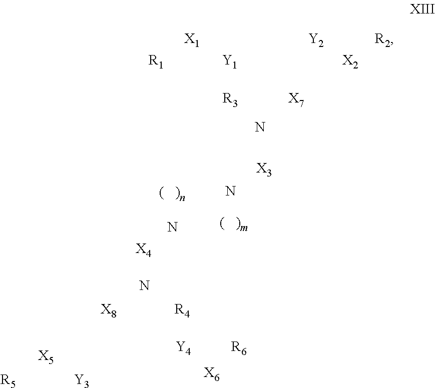
or an isomer, or a salt thereof, wherein:
R₁, R₂, R₃, R₄, R₅, and R₆ are the same or different and are independently selected from hydrogen, halogen, alkyl, alkenyl, alkynyl, carbocyclyl, heterocyclyl, aryl, and heteroaryl, each except hydrogen and halogen optionally substituted;
X₁, X₂, X₃, X₄, X₅, and X₆ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkenylene, alkynylene, and cyclic alkylene or heteroalkylenes, wherein R at each occurrence is independently hydrogen, or an alkyl, alkenyl, alkynyl, carbocyclyl, heterocyclyl, group;
X₇ and X₈ are the same or different and are independently selected from —(C═O)O—, —O—(C═O)—, —(C═O)S—, —S(C═O)—, —NR(C═O)—, —(C═O)NR—, —O—, —S—, —S—S—, —CH₂—, —NR—, alkenylene, alkynylene, and cyclic alkylene or heteroalkylenes, wherein R is independently hydrogen, or an alkyl, alkenyl, alkynyl, carbocyclyl, heterocyclyl, group;
or X₇ and X₈ are each a linear alkyl group comprising 0 to 10 methylene units;
Y₁, Y₂, Y₃, and Y₄ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, alkynylenes, and cyclic alkylene or heteroalkylenes, wherein R at each occurrence is independently hydrogen, or an alkyl, alkenyl, alkynyl, carbocyclyl, heterocyclyl, group;
the central ring structure is an optionally substituted aromatic, heteroaromatic, non-aromatic, or anti-aromatic ring system; and
n and m are independently selected from 0, 1, 2, 3, 4, 5, 6, 7, 8, 9, and 10,
wherein the longest chain of atoms in the compound is between 18 to 70 atoms; and 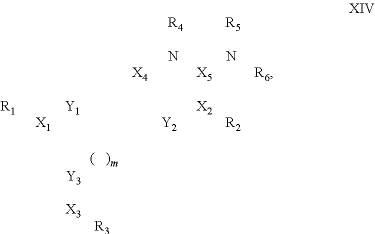
or an isomer, or a salt thereof, wherein:
R₁, R₂, R₃, R₄, R₅, and R₆ the same or different and are independently selected from hydrogen, halogen, alkyl, alkenyl, alkynyl, carbocyclyl, heterocyclyl, aryl, and heteroaryl, each except hydrogen and halogen optionally substituted;
X₁, X₂, X₃, X₄, and X₅ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, —NR—, alkylene, alkenylene, alkynylene, cycloalkylene, and heterocyclylene, wherein R is independently hydrogen, alkyl, alkenyl, alkynyl, carbocyclyl, and heterocyclyl;
Y₁, Y₂, and Y₃ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkylene, alkenylene, alkynylene, cycloalkylene, and heterocyclylene, wherein R is independently hydrogen, alkyl, alkenyl, alkynyl, carbocyclyl, or heterocyclyl; and
m is selected from 0, 1, 2, 3, 4, 5, 6, 7, 8, 9, and 10,
wherein the longest chain of atoms in the compound is between 18 to 70 atoms.

2. (canceled)

3. (canceled)

4. The compound of Formula II of claim 1: 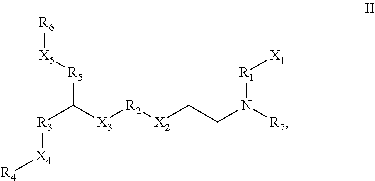
or an isomer, or a salt thereof, wherein:
R₁ is a C₁₋₁₈ alkylene, C₂₋₁₈ alkenylene, or C₂₋₁₈ alkynylene;
R₂ is a C₁₋₁₈ alkylene, C₂₋₁₈ alkenylene, or C₂₋₁₈ alkynylene, C₃₋₈ carbocyclylene, 3- to 8-membered heterocyclylene, or C₁₋₁₈ heteroalkylene;
R₃ and R₅ are the same or different, each independently a bond or a C₁₋₂₈ alkylene, C₂₋₂₈ alkenylene, or C₂₋₂₈ alkynylene;
R₄, R₆, and R₇ are the same or different, each independently hydrogen or a C₁₋₂₈ alkyl, C₂₋₂₈ alkenyl, or C₂₋₂₈ alkynyl, each optionally substituted by one, two, or three substituents independently selected from —OR, —SR, —S—S—R, —O—CO—R, —CO—OR, —CO—NR^{a}R^{b}, —NR^{a}—CO—R, —O—CO—NR^{a}R^{b}, —NR^{a}—CO—OR, —NR^{a}R^{b}, and —S—CO—R, wherein R at each occurrence is independently hydrogen or a methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, or icosyl; and R^{a} and R^{b} are independently hydrogen or lower alkyl;
X₁ is —OH, —SH, —N(R)₂, C₅₋₈ carbocyclyl, heterocyclyl, or hydrogen, or absent, wherein R at each occurrence is independently a lower alkyl or hydrogen;
X₂ is —O—CO—, —CO—O—, —NR—CO—, or —CO—NR—, wherein R is a lower alkyl or hydrogen; and
X₃, X₄, and X₅ are each independently —O—CO—, —CO—O—, —NR—CO—, —CO—NR—, a bond, or absent, wherein R is a lower alkyl or hydrogen,
wherein the longest chain of atoms in the compound is between 18 and 70 atoms.

5. The compound of claim 4, or an isomer, or a salt thereof, wherein R₅ is a bond; and R₆ is hydrogen; and X₅ is absent.

6. The compound of claim 4, or an isomer, or a salt thereof, wherein X₂ is —O—CO— or —CO—O—.

7. The compound of claim 4, or an isomer, or a salt thereof, wherein R₇ is a linear C₁₈ alkenyl.

8. The compound of claim 4, or an isomer, or a salt thereof, wherein R₁ and X₁ together are a 1,2-dihydroxypropane moiety, pyrrolidinoethylamine moiety, or (2-hydroxyethyl)(ethyl)amino)ethylamine moiety.

9. The compound of claim 4, or an isomer, or a salt thereof, wherein R₇ is a 2-hexyldecyl hexanoate moiety; X₂ is —O—CO— or —CO—O—; and R₂ is methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, nonadecyl, icosyl, or ((2-hexyldecyl)thio)ethyl.

10. The compound of claim 4, or an isomer, or a salt thereof, wherein:
R₁ is a C₁₋₈ alkylene, C₂₋₈ alkenylene, or C₂₋₈ alkynylene;
R₂ is a C₁₋₈ alkylene, C₂₋₈ alkenylene, C₂₋₈ alkenylene, or C₁₋₈ heteroalkylene;
R₃ and R₅ are the same or different, each independently a bond or a C₁₋₁₄ alkylene, C₂₋₁₄ alkenylene, or C₂₋₁₄ alkenylene;
R₄, R₆, and R₇ are the same or different, each independently a hydrogen or a C₁₋₁₄ alkylene, C₂₋₁₄ alkenylene, or C₂₋₁₄ alkynylene;
X₁ is —OH, —N(R)₂, a C₅₋₈ carbocycyl or a hydrogen, wherein R at each occurrence is independently a lower alkyl or hydrogen;
X₂ is —O—CO— or —CO—O—; and
X₃, X₄, and X₅ are each independently, a bond, —O—CO—, or —CO—O—,
wherein the longest chain of atoms in the compound is between 18 and 70 atoms.

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. The compound of Formula VI of claim 1: 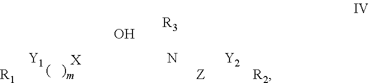
or an isomer, or a salt thereof, wherein:
Y₁ and Y₂ are the same or different, each independently selected from —(C═O)O—, —O(C═O)—, —(C═O)S—, —S(C═O)—, —NR(C═O)—, —(C═O)NR—, —O—, —S—, —S—S—, —CH₂—, an alkenylene, and an alkynylene, wherein R is independently a hydrogen, or an alkyl, alkenyl, alkynyl, carbocyclyl, heterocyclyl, group;
X is selected from —S—, —S—S—, —O—, —CH₂—, alkenylene, and alkynylene, —NR—, wherein R is hydrogen or an alkyl, alkenyl, alkynyl, carbocyclyl, heterocyclyl, group;
R₁ and R₂ are the same or different, each independently selected from carbocyclyl, alkyl, alkenyl, and alkynyl, each optionally substituted;
R₃ is selected from hydrogen, halogen, alkyl, alkenyl, alkynyl, carbocyclyl, heterocyclyl, aryl, and heteroaryl, each, except hydrogen and halogen, optionally substituted;
Z is selected from alkylene, alkenylene, alkynylene, and carbocyclylene; and
m is an integer selected from 1 to 24,
wherein the longest chain of atoms in the compound is between 18 to 70 atoms.

28. The compound of claim 27, or an isomer, or a salt thereof, wherein Y₁ and Y₂ are independently selected from —(C═O)O—, —O(C═O)—, —(C═O)S—, —S(C═O)—, —NR(C═O)—, and —(C═O)NR—.

29. The compound of claim 27, or an isomer, or a salt thereof, wherein R₃ is selected from or an alkyl, alkenyl, alkynyl, carbocyclyl, heterocyclyl, aryl, or heteroaryl, each optionally substituted by —OH.

30. The compound of claim 27, or an isomer, or a salt thereof, wherein X is oxygen (—O—) or substituted or unsubstituted nitrogen (—NR—).

31. The compound of claim 27, or an isomer, or a salt thereof, wherein:
Y₁ and Y₂ are the same or different, each independently selected from —(C═O)O—, —O(C═O)—, —NR(C═O)—, —(C═O)NR—, —O—, —CH₂—, an alkenylene, and an alkynylene, wherein R is independently a hydrogen, or an alkyl, alkenyl, alkynyl, carbocyclyl, heterocyclyl, group;
X is selected from —S—, —O—, —CH₂—, alkenylene, and alkynylene, —NR—, wherein R is hydrogen or an alkyl, alkenyl, alkynyl, carbocyclyl, heterocyclyl, group;
R₁ and R₂ are the same or different, each independently selected from carbocyclyl, alkyl, alkenyl, and alkynyl, each optionally substituted;
R₃ is selected from hydrogen, alkyl, alkenyl, alkynyl, carbocyclyl, heterocyclyl, aryl, and heteroaryl, each, except hydrogen, optionally substituted;
Z is selected from alkylene, alkenylene, alkynylene, and carbocyclylene; and
m is 1 to 14,
wherein the longest chain of atoms in the compound is between 18 to 70 atoms.

32. The compound of formula VII of claim 1: 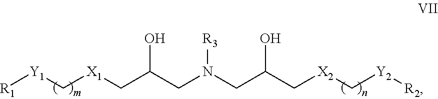
or an isomer, or a salt thereof, wherein:
Y₁ and Y₂ are the same or different and are independently selected from —(C═O)O—, —O(C═O)—, —(C═O)S—, —S(C═O)—, —NR(C═O)—, —(C═O)NR—, —O—, —S—, —S—S—, —CH₂—, alkenylene, and alkynylene, wherein R at each occurrence is independently hydrogen, or an alkyl, alkenyl, alkynyl, carbocyclyl, heterocyclyl, group;
X₁ and X₂ are the same or different and are independently selected from —S—, —S—S—, —O—, —CH₂—, alkenylene, alkynylene, and —NR—, wherein R is hydrogen, or an alkyl, alkenyl, alkynyl, carbocyclyl, heterocyclyl, group;
R₁ and R₂ are the same or different and independently selected from alkyl, alkenyl, alkynyl, and heterocyclyl, each optionally substituted;
R₃ is selected from hydrogen, halogen, alkyl, alkenyl, alkynyl, carbocyclyl, heterocyclyl, aryl, and heteroaryl groups, each optionally substituted; and
m and n are each independently an integer selected from 1 to 24,
wherein the longest chain of atoms in the compound is between 18 to 70 atoms.

33. The compound of claim 32, or an isomer, or a salt thereof, wherein X₁ and X₂ are each independently oxygen (—O—), sulfur (—S—), or substituted or unsubstituted nitrogen (—NR—) or methylene.

34. The compound of claim 32, or an isomer, or a salt thereof, wherein Y₁ and Y₂ are independently selected from —(C═O)O—, —O(C═O)—, —(C═O)S—, —S(C═O)—, —NR(C═O)—, and —(C═O)NR—.

35. The compound of claim 32, or an isomer, or a salt thereof, wherein R₃ is selected from alkyl, cycloalkyl, or aryl, each optionally substituted by —OH.

36. The compound of claim 32, or an isomer, or a salt thereof, wherein:
Y₁ and Y₂ are the same or different, each independently selected from —(C═O)O—, —O(C═O)—, —NR(C═O)—, —(C═O)NR—, —O—, —CH₂—, an alkenylene, and an alkynylene, wherein R is independently a hydrogen, or an alkyl, alkenyl, alkynyl, carbocyclyl, heterocyclyl, group;
X₁ and X₂ are the same or different and are independently selected from —S—, —O—, —CH₂—, alkenylene, and alkynylene, —NR—, wherein R is hydrogen or an alkyl, alkenyl, alkynyl, carbocyclyl, heterocyclyl, group;
R₁ and R₂ are the same or different, each independently selected from carbocyclyl, alkyl, alkenyl, and alkynyl, each optionally substituted;
R₃ is selected from hydrogen, alkyl, alkenyl, alkynyl, carbocyclyl, heterocyclyl, aryl, and heteroaryl, each, except hydrogen, optionally substituted; and
m and n are each independently an integer selected from 1 to 14,
wherein the longest chain of atoms in the compound is between 18 and 70 atoms.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. A compound of Formula I, II, III, IV, V, VI, VII, VIII, IX, X, XI, XII, XIII, or XIV selected from the Compounds of List 1 through the Compounds of List 14, an isomer, or a salt thereof.

65. A lipid nanoparticle composition comprising:
a. a biological and/or therapeutic agent with an N:P Ratio of 1 to 15;
b. a compound of claim 1 according to Formula I, II, III, IV, V, VI, VII, VIII, IX, X, XI, XII, XIII, or XIV, or an isomer, or a salt thereof, constituting from 10 mol % to 85 mol % of the total lipid present in the composition;
c. a neutral “helper” phospholipid or a derivative thereof, constituting from 5 mol % to 40 mol % of the total lipid in the composition;
d. cholesterol, or a derivative thereof, constituting from 10 mol % to 50 mol % of the total lipid in the composition;
e. a conjugated lipid that inhibits aggregation constituting from 0 mol % to 10 mol % of the total lipid in the composition.

66. The lipid nanoparticle composition of claim 65, wherein the phospholipid is selected from 1,2-dilinoleoyl-sn-glycero-3-phosphocoline (DLPC), 1,2-dimyristoyl-sn-glycero-phophocholine (DMPC), 1,2-dioleoyl-sn-glycero-3-phosphocholine (DOPC), 1,2-dipalmitoyl-sn-glycero-3-phophocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-dioleoyl-sn-glycero-3-phophoethanolamine (DOPE), palmitoyloleoyl-phosphatidylethanolamine (POPE), 1,2-diphytanoyl-sn-glycero-3-phosphoethanolamine, 1,2-dioleoyl-snglycero-3-phospho-rac-(1-glycerol) sodium salt (DOPG), sphingomyelin, and mixtures thereof.

67. The lipid nanoparticle composition of claim 65, wherein the PEG-lipid is selected from PEG-modified phosphatidylethanolamines, PEG-modified phosphatidic acids, PEG-modified ceramides, PEG-modified dialkylamines, PEG-modified diacylglycerols, PEG-modified dialkylglycerols, PEG-modified glycerides, PEG-modified sterols, and mixtures thereof.

68. The lipid nanoparticle composition of claim 65, wherein the biological/therapeutic agent is a ribonucleic acid (RNA), wherein optionally the RNA is selected from a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), self-amplifying mRNA (sa mRNA) and mixtures thereof.

69. The lipid nanoparticle composition of claim 65, wherein the biological and/or therapeutic agent comprises a mRNA, wherein optionally the mRNA comprises from about 300 to about 20,000 nucleotides; and wherein optionally the mRNA comprises at least one modified nucleotide.

70. The lipid nanoparticle composition of claim 65, wherein the neutral phospholipid comprises distearoylphosphatidylcholine (DSPC).

71. The lipid nanoparticle composition of claim 65, wherein the conjugated lipid that inhibits aggregation of particles comprises a polyethylene glycol-lipid conjugate (PEG-lipid), wherein optionally the PEG-lipid conjugate comprises 1,2-dimyristoyl-rac-glycero-3-methoxypolyethylene glycol (DMG-PEG), and wherein optionally the PEG has an average molecular weight of 2000 Daltons.

72. The lipid nanoparticle composition of claim 65, wherein the biological and/or therapeutic agent is an oligonucleotide, wherein optionally the oligonucleotide comprises from about 10 to about 200 nucleotides; wherein optionally the oligonucleotide comprises one or more modified nucleotides; and wherein optionally the oligonucleotide comprises at least one 2′-O-methyl (2′OMe) nucleotide.

73. A method of treating a disease or disorder, comprising administering to a subject in need of treatment a therapeutically effective amount of a lipid nanoparticle composition according to claim 65, wherein optionally the disease or disorder is selected from leber congenital amaurosis, Alzheimer's disease, Parkinson's disease, cystic fibrosis, Fabry disease, SMN1-related spinal muscular atrophy, Huntington's disease, muscular dystrophies (such as Dunchenne and Becker), human immunodeficiency virus (HIV), influenza, heart disease, cancers (such as e.g. breast, prostate, colorectal, renal, bladder, lymphomas, thyroid, endometrial, pancreatic), tuberculosis, multiple sclerosis, transthyretin amyloidosis, hemophilia diseases (such as, e.g., hemophilia B, hemophilia A), amyotrophic lateral sclerosis, GALT-related galcosemia, VEGF-related heart failure, propionic acidemia, ornithine transcarbamylase deficiency, Zika virus, rabies, SARS-CoV-2, malaria, tuberculosis, Hepatitis B, Gaucher's disease, Creutzfeldt-Jakob disease, nephrogenic diabetes insipidus, spinocerebellar ataxia, Dentatorubral-pallidoluysian atrophy, Sickle cell anemia, Machado-Joseph atrophy, retinitis pigmentosa, α-Antitrypsin deficiency, galactocerebrosidase deficiencies, Bardet-Biedel syndrome, Charlevoix-Daguenay, ethylmalonic aciduria, familial hypercholesterilemia, leprechaunism, Marfan syndrome, McKusick-Kaufman syndrome, Osteogenesis imperfecta, phenylketonuria, Tay-Sachs disease, cataracts, familial amyloidosis, Wilson's disease, Santavuori-Haltia disease, Jansky-Bielschowsky disease, Juvenile Batten disease, Juvenile Neuronal Ceroid Lipofuscinosis, and Pelizaeus-Merzbacher disease.
